(19) United States
(12) Patent Application Publication
(10) Pub. No.: US 2025/0287838 A1
(43) Pub. Date: Sep. 11, 2025

(54) HETEROCYCLIC COMPOUND, ORGANIC LIGHT-EMITTING ELEMENT COMPRISING SAME, AND COMPOSITION FOR ORGANIC MATERIAL LAYER

(71) Applicant: LT MATERIALS CO.,LTD., Yongin-si (KR)

(72) Inventors: Cheol-Hun JEONG, Yongin-si (KR); Geon-Yu PARK, Yongin-si (KR); Young-Seok NO, Yongin-si (KR); Dong-Jun KIM, Yongin-si (KR); Dae-Hyuk CHOI, Yongin-si (KR)

(21) Appl. No.: 19/218,623

(22) Filed: May 27, 2025

Related U.S. Application Data
(63) Continuation of application No. PCT/KR2023/017206, filed on Nov. 1, 2023.

(30) Foreign Application Priority Data
Dec. 13, 2022 (KR) 10-2022-0173996

(51) Int. Cl. H10K 85/60; C07D 403/14; C07D 405/14; C07D 409/14; C09K 11/02; H10K 50/11

(52) U.S. Cl. CPC H10K 85/654; C07D 403/14; C07D 405/14; C07D 409/14; C09K 11/02; H10K 85/615; H10K 85/622; H10K 85/6572; H10K 85/6574; H10K 85/6576; H10K 50/11

(57) ABSTRACT

Provided is a heterocyclic compound represented by Chemical Formula 1, and an organic light emitting device and a composition for an organic material layer, including the same. The heterocyclic compound can be used as a material for the organic material layer of the organic light emitting device. The compound may serve as a hole injection material, a hole transport material, a light emitting material, an electron transport material, an electron injection material, and the like in an organic light emitting device. In particular, the compound can be used as a material for a light emitting layer of an organic light emitting device.

### TECHNICAL FIELD

This application claims priority to and the benefit of Korean Patent Application No. 10-2022-0173996 filed in the Korean Intellectual Property Office on Dec. 13, 2022, the entire contents of which are incorporated herein by reference.

The present specification relates to a heterocyclic compound, an organic light emitting device including the same and a composition for an organic material layer.

### BACKGROUND ART

An electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may perform a function such as hole injection, hole transport, electron blocking, hole blocking, electron transport or electron injection.

In order to improve the performance, service life, or efficiency of the organic light emitting device, there is a continuous need for developing a material for an organic thin film.

### RELATED ART DOCUMENT

U.S. Pat. No. 4,356,429

### DETAILED DESCRIPTION OF THE INVENTION

### Technical Problem

The present invention has been made in an effort to provide a heterocyclic compound, an organic light emitting device including the same, a method for manufacturing the same, and a composition for an organic material layer.

### Technical Solution

In an exemplary embodiment of the present application, provided is a heterocyclic compound represented by the following Chemical Formula 1.

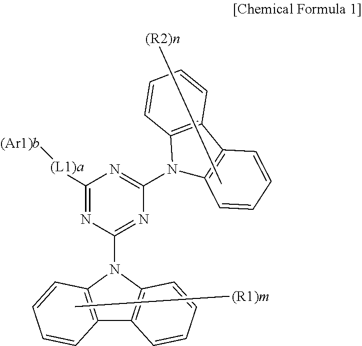

In Chemical Formula 1,
R1 and R2 are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more adjacent groups are bonded to each other to form a substituted or unsubstituted C6 to C60 aromatic hydrocarbon ring or a substituted or unsubstituted C2 to C60 hetero ring, m and n are each an integer from 1 to 8, and when m is 2 or higher, R1's are the same as or different from each other, and when n is 2 or higher, R2's are the same as or different from each other,
L1 is a direct bond; a substituted or unsubstituted C6 to C60 arylene group; or a substituted or unsubstituted C2 to C60 heteroarylene group, a is an integer from 1 to 4, and when a is 2 or higher, L1's are the same as or different from each other,
Ar1 is a substituted or unsubstituted C1 to C60 alkyl group; a substituted or unsubstituted C6 to C60 aryl group; or a substituted or unsubstituted C2 to C60 heteroaryl group including O or S,
b is an integer from 1 to 4, and when b is 2 or higher, Ar1's are the same as or different from each other, and
a deuterium content of

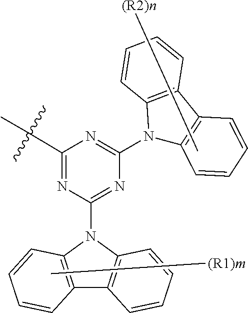

of Chemical Formula 1 is 20% or more and 100% or less.

Further, in an exemplary embodiment of the present application, provided is an organic light emitting device including: a first electrode; a second electrode provided to face the first electrode; and an organic material layer having one or more layers provided between the first electrode and the second electrode, in which one or more layers of the organic material layer include the heterocyclic compound represented by Chemical Formula 1.

In addition, an exemplary embodiment of the present application provides an organic light emitting device in which an organic material layer including the heterocyclic compound of Chemical Formula 1 further includes a heterocyclic compound represented by the following Chemical Formula 2 or 3.

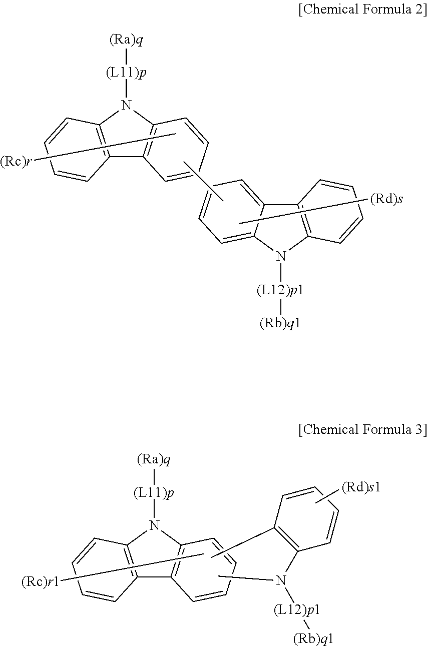

In Chemical Formulae 2 and 3,
Rc and Rd are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group,
L11 and L12 are the same as or different from each other, and are each independently a direct bond; a substituted or unsubstituted C6 to C60 arylene group; or a substituted or unsubstituted C2 to C60 heteroarylene group,
Ra and Rb are the same as or different from each other, and are each independently a substituted or unsubstituted C6 to C60 aryl group; a substituted or unsubstituted C2 to C60 heteroaryl group; a cyano group; or —SiRR′R″,
R, R′, and R″ are the same as or different from each other, and are each independently hydrogen; deuterium; —CN; a substituted or unsubstituted C1 to C60 alkyl group; a substituted or unsubstituted C6 to C60 aryl group; or a substituted or unsubstituted C2 to C60 heteroaryl group,
p, p1, q, and q1 are an integer from 1 to 4,
r and s are an integer from 0 to 7,
r1 is an integer from 0 to 4,
s1 is an integer from 0 to 6, and
when p, p1, q, q1, r, s, r1, and s1 are 2 or higher, substituent in the parenthesis are the same as or different from each other.

Finally, in an exemplary embodiment of the present application, provided is a composition for an organic material layer of an organic light emitting device, including: a heterocyclic compound represented by Chemical Formula 1 according to the present application; and a heterocyclic compound represented by Chemical Formula 2 or 3.

### Advantageous Effects

The compound described in the present specification can be used as a material for the organic material layer of the organic light emitting device. The compound may serve as a hole injection material, a hole transport material, a light emitting material, an electron transport material, an electron injection material, and the like in an organic light emitting device. In particular, the compound can be used as a material for a light emitting layer of an organic light emitting device.

Specifically, the compound may also be used alone as a light emitting material, and as a host material or a dopant material of the light emitting layer. When the compound represented by Chemical Formula 1 is used for an organic material layer, the driving voltage of the device can be lowered, the light efficiency of the device can be improved, and the service life characteristics of the device can be improved due to the thermal stability of the compound.

In particular, the heterocyclic compound represented by Chemical Formula 1 and the heterocyclic compound represented by Chemical Formula 2 or 3 can be used simultaneously as a material for a light emitting layer of an organic light emitting device. In this case, it is possible to lower a driving voltage of the device, improve the light efficiency, and particularly improve the service life characteristics of the device by the thermal stability of the compound.

### BRIEF DESCRIPTION OF DRAWINGS

FIGS. 1 to 3 each are views schematically illustrating a stacking structure of an organic light emitting device according to an exemplary embodiment of the present application.

### EXPLANATION OF REFERENCE NUMERALS AND SYMBOLS

100: Substrate
200: Positive electrode
300: Organic material layer
301: Hole injection layer
302: Hole transport layer
303: Light emitting layer
304: Hole blocking layer
305: Electron transport layer
306: Electron injection layer
400: Negative electrode

### BEST MODE

Hereinafter, the present specification will be described in more detail.

When one part “includes” one constituent element in the present specification, unless otherwise specifically described, this does not mean that another constituent element is excluded, but means that another constituent element may be further included.

In the present specification,

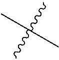

of a chemical formula means a position to which a constituent element is bonded. That is,

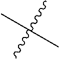

indicates a position where chemical formulae are linked to each other.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; a cyano group; a C1 to C60 alkyl group; a C2 to C60 alkenyl group; a C2 to C60 alkynyl group; a C3 to C60 cycloalkyl group; a C2 to C60 heterocycloalkyl group; a C6 to C60 aryl group; a C2 to C60 heteroaryl group; a silyl group; a phosphine oxide group; and an amine group, or with a substituent to which two or more substituents selected among the exemplified substituents are bonded.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and may also be interpreted as an element which has the same number of protons, but different number of neutrons.

In an exemplary embodiment of the present application, when the total number of substituents of a basic compound is defined as T1 and the number of specific substituents among the substituents is defined as T2, the content T % of the specific substituent may be defined as T2/T1×100=T %.

That is, in an example, the deuterium content of 20% in a phenyl group represented by

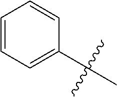 [SMILES: *c1ccccc1]

may be represented by 20% when the total number of substituents that the phenyl group can have is 5 (T1 in the formula) and the number of deuterium atoms among the substituents is 1 (T2 in the formula). That is, a deuterium content of 20% in the phenyl group may be represented by the following structural formula.

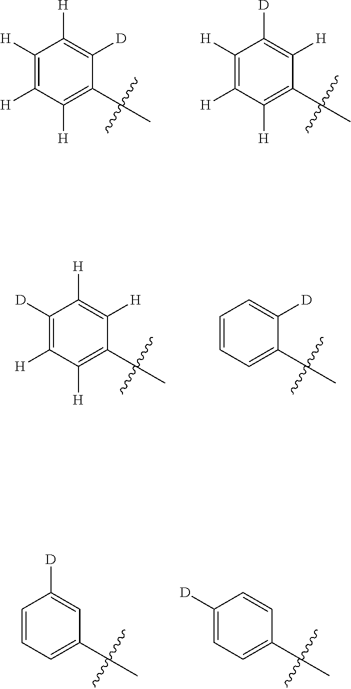 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, the alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, the alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto.

In the present specification, the alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, an alkoxy group may be straight-chained, branched, or cyclic. The number of carbon atoms of the alkoxy group is not particularly limited, but is preferably 1 to 20. Specific examples thereof include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but are not limited thereto.

In the present specification, the cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, the heterocycloalkyl group includes 0, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, the aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

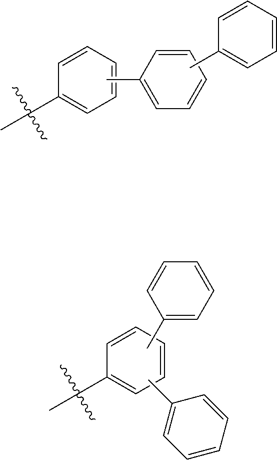

In the present specification, the fluorenyl group may be substituted, and adjacent substituents may be bonded to each other to form a ring.

When the fluorenyl group is substituted, the substituent may be the following structure, but is not limited thereto.

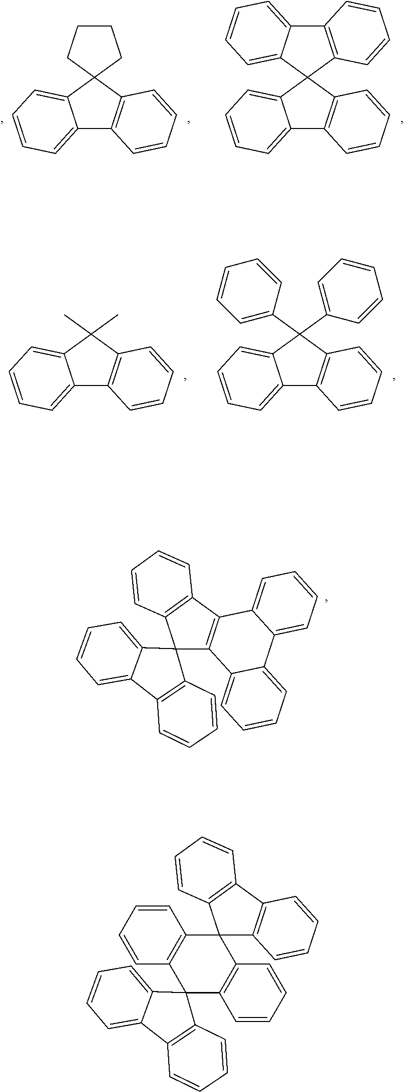

In the present specification, the heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolilyl group, a naphthyridyl group, an acridinyl group, a phenanthridinyl group, an imidazopyridinyl group, a diaza naphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi (dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepin group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, 2,3-dihydrobenzo[b]thiophene, 2,3-dihydrobenzofuran, a 5,10-dihydrodibenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like, but are not limited thereto.

In the present specification, a silyl group includes Si and is a substituent to which the Si atom is directly linked as a radical, and is represented by —Si(R101)(R102)(R103), and R101 to R103 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specific examples of the silyl group include

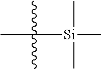 [SMILES: *[Si](C)(C)C]

(a trimethyl silyl group),

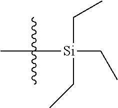 [SMILES: *[Si](CC)(CC)CC]

(a triethylsilyl group),

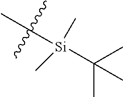 [SMILES: *[Si](C)(C)C(C)(C)C]

(a t-butyldimethylsilyl group),

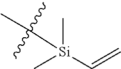 [SMILES: *[Si](C)(C)C=C]

(a vinyldimethylsilyl group),

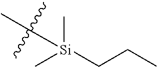 [SMILES: *[Si](C)(C)CCC]

(a propyldimethylsilyl group),

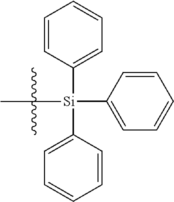 [SMILES: *[Si](c1ccccc1)(c1ccccc1)c1ccccc1]

(a triphenylsilyl group),

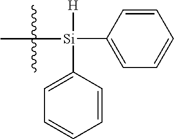 [SMILES: *[SiH](c1ccccc1)c1ccccc1]

(a diphehylsilyl group),

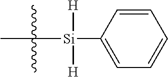 [SMILES: *[SiH2]c1ccccc1]

(a phenylsilyl group) and the like, but are not limited thereto.

In the present specification, the phosphine oxide group is represented by —P(═O)(R104)(R105), and R104 and R105 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide, and the like, but are not limited thereto.

In the present specification, the amine group is represented by —N(R106)(R107), and R106 and R107 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described examples of the aryl group may be applied to an arylene group except for a divalent arylene group.

In the present specification, the above-described examples of the heteroaryl group may be applied to a heteroarylene group except for a divalent heteroarylene group.

An exemplary embodiment of the present specification provides the heterocyclic compound represented by Chemical Formula 1.

In an exemplary embodiment of the present application, R1 and R2 are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more adjacent groups are bonded to each other to form a substituted or unsubstituted C6 to C60 aromatic hydrocarbon ring or a substituted or unsubstituted C2 to C60 hetero ring.

In another exemplary embodiment, R1 and R2 are the same as or different from each other, and may be each independently selected from the group consisting of hydrogen; deuterium; a halogen; a cyano group; a substituted or unsubstituted C1 to C60 alkyl group; a substituted or unsubstituted C6 to C60 aryl group; and a substituted or unsubstituted C2 to C60 heteroaryl group.

In still another exemplary embodiment, R1 and R2 are the same as or different from each other, and may be each independently selected from the group consisting of hydrogen; deuterium; a halogen; a cyano group; a substituted or unsubstituted C1 to C40 alkyl group; a substituted or unsubstituted C6 to C40 aryl group; and a substituted or unsubstituted C2 to C40 heteroaryl group.

In yet another exemplary embodiment, R1 and R2 are the same as or different from each other, and may be each independently selected from the group consisting of hydrogen; deuterium; a halogen; a cyano group; a substituted or unsubstituted C1 to C40 alkyl group; a substituted or unsubstituted C6 to C20 aryl group; or a substituted or unsubstituted C2 to C20 heteroaryl group.

In still yet another exemplary embodiment, R1 and R2 are the same as or different from each other, and may be each independently selected from the group consisting of hydrogen; deuterium; a halogen; a cyano group; a C1 to C40 alkyl group unsubstituted or substituted with deuterium; a C6 to C20 aryl group unsubstituted or substituted with deuterium; and a C2 to C20 heteroaryl group unsubstituted or substituted with deuterium.

In a further exemplary embodiment, R1 and R2 are the same as or different from each other, and may be each independently hydrogen; deuterium; or a C6 to C20 aryl group unsubstituted or substituted with deuterium.

In another further exemplary embodiment, R1 and R2 are the same as or different from each other, and may be each independently hydrogen; deuterium; a monocyclic C6 to C10 aryl group unsubstituted or substituted with deuterium.

In still another further exemplary embodiment, R1 and R2 are the same as or different from each other, and may be each independently hydrogen; deuterium; or a substituted or unsubstituted phenyl group.

In yet another further exemplary embodiment, R1 and R2 are the same as or different from each other, and may be each independently hydrogen; deuterium; or a phenyl group unsubstituted or substituted with deuterium.

In an exemplary embodiment of the present application,

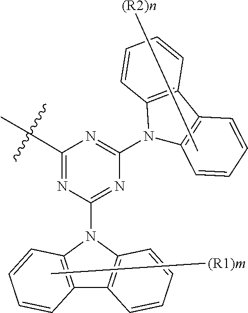

of Chemical Formula 1 may be represented by any one of the following Chemical Formulae 4 to 17.

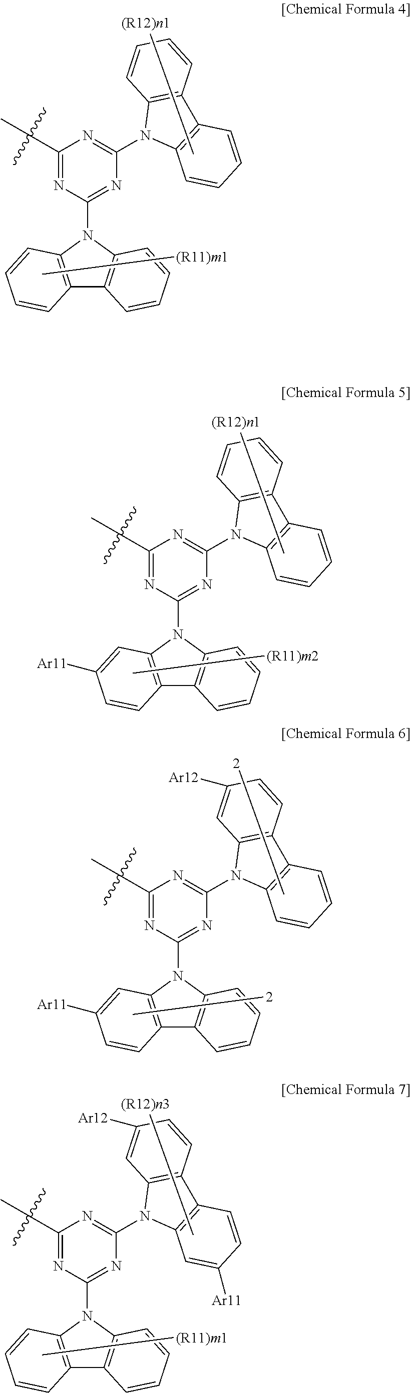

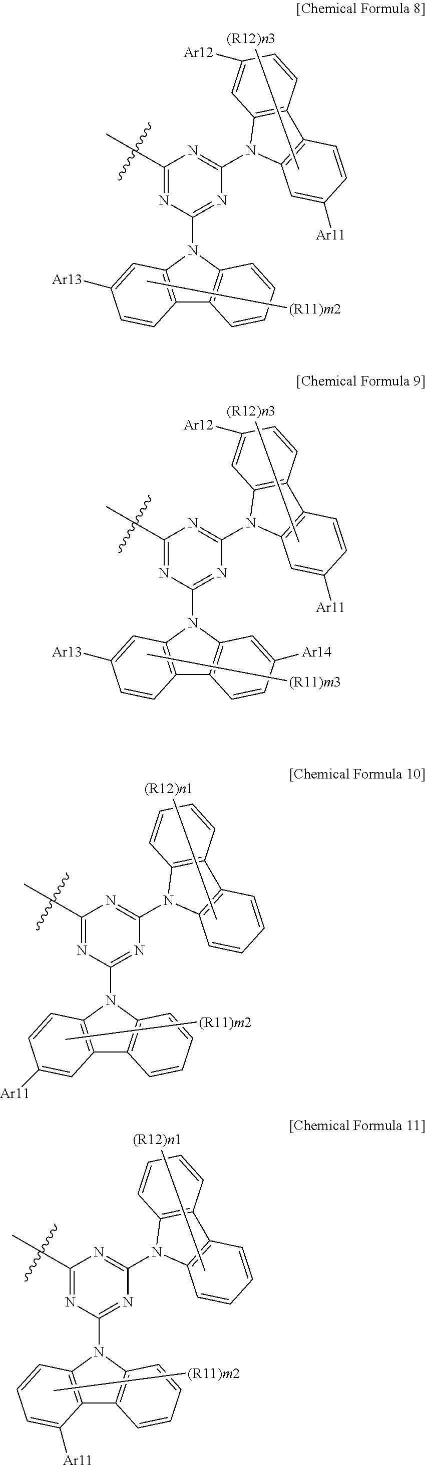

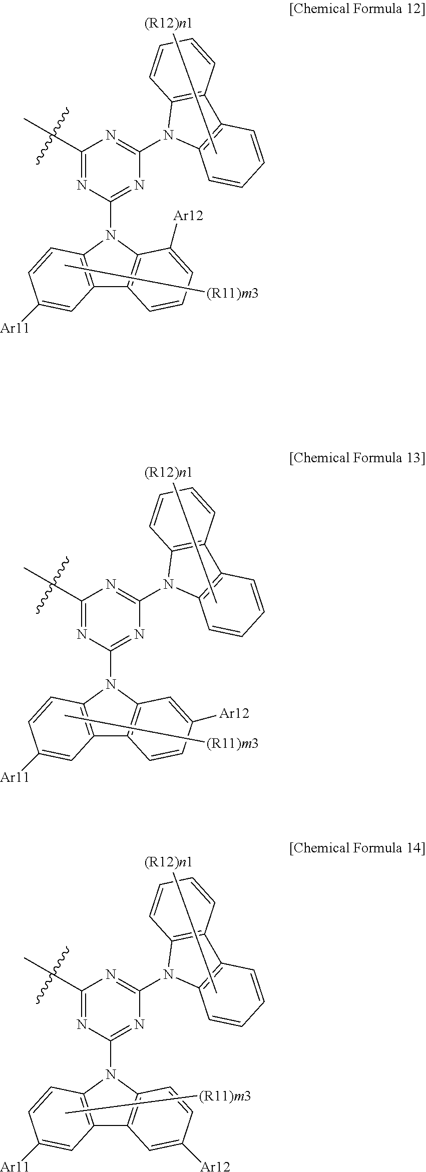

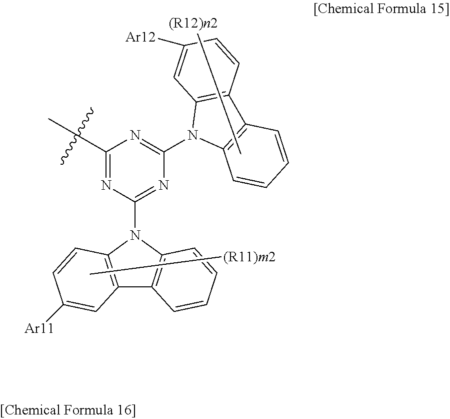

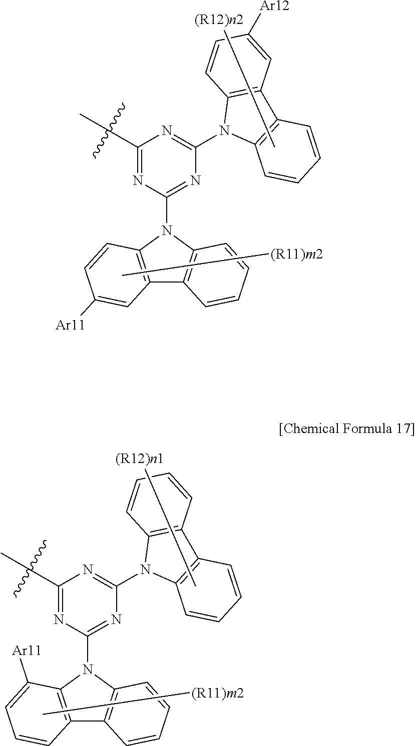

In Chemical Formulae 4 to 17,
R11 and R12 are the same as or different from each other, and are each independently hydrogen or deuterium,
   Ar11 to Ar14 are the same as or different from each other, and are each independently a substituted or unsubstituted C6 to C60 aryl group; or a substituted or unsubstituted C2 to C60 heteroaryl group,
m1 and n1 are an integer from 0 to 8,
m2 and n2 are an integer from 0 to 7,
m3 and n3 are an integer from 0 to 6, and
when m1, m2, m3, n1, n2, and n3 are each 2 or higher, substituents in the parenthesis are the same as or different from each other.

In an exemplary embodiment of the present application, the deuterium content of

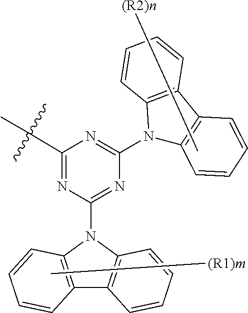

of Chemical Formula 1 is 20% or more and 100% or less.

In another exemplary embodiment, the deuterium content of

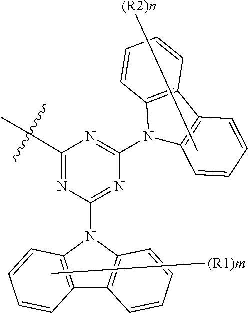

of Chemical Formula 1 may be 20% or more and 100% or less, 25% or more and 100% or less, 30% or more and 100% or less, 50% or more and 100% or less, 60% or more and 100% or less, 70% or more and 100% or less, and 80% or more and 100% or less.

The deuterium content of

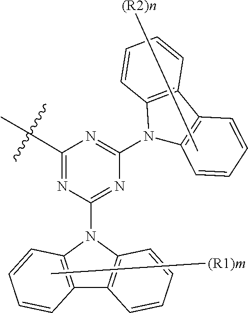

of Chemical Formula 1 falling within the above range may refer to the deuterium content of not only the above structural formula, but also the substituent when the above structural formula has a substituent.

In an exemplary embodiment of the present application, Chemical Formula 1 is represented by each of the following Structural Formulae A and B,

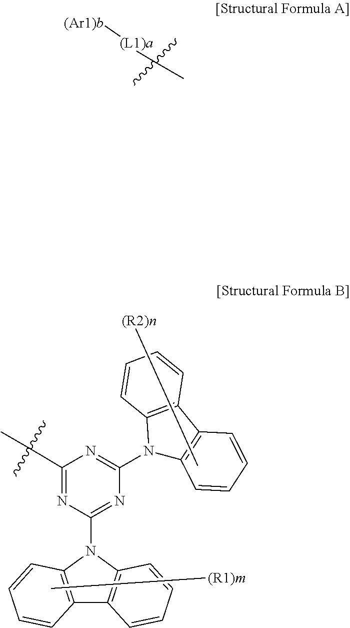

The deuterium content of Structural Formula A may be 10% or less, and
the deuterium content of Structural Formula B may be 20% or more and 100% or less.

In an exemplary embodiment of the present application, Structural Formula B may include the above-described deuterium content.

In an exemplary embodiment of the present application, the deuterium content of Structural Formula A may be 10% or less, preferably 5% or less, and 0% or more and 3% or less.

In an exemplary embodiment of the present application, the deuterium content of Chemical Formula 1 may be 10% or more and 100% or less.

In an exemplary embodiment of the present application, Ar11 to Ar14 are the same as or different from each other, and are each independently a substituted or unsubstituted C6 to C60 aryl group; or a substituted or unsubstituted C2 to C60 heteroaryl group.

In another exemplary embodiment, Ar11 to Ar14 are the same as or different from each other, and are each independently a substituted or unsubstituted C6 to C40 aryl group; or a substituted or unsubstituted C2 to C40 heteroaryl group.

In still another exemplary embodiment, Ar11 to Ar14 are the same as or different from each other, and are each independently a substituted or unsubstituted C6 to C20 aryl group; or a substituted or unsubstituted C2 to C20 heteroaryl group.

In yet another exemplary embodiment, Ar11 to Ar14 are the same as or different from each other, and are each independently a C6 to C20 aryl group unsubstituted or substituted with deuterium; or a C2 to C20 heteroaryl group unsubstituted or substituted with deuterium.

In still yet another exemplary embodiment, Ar11 to Ar14 are the same as or different from each other, and are each independently a C6 to C20 aryl group unsubstituted or substituted with deuterium.

In a further exemplary embodiment, Ar11 to Ar14 are the same as or different from each other, and are each independently a substituted or unsubstituted phenyl group.

In another further exemplary embodiment, Ar11 to Ar14 are the same as or different from each other, and are each independently a phenyl group unsubstituted or substituted with deuterium.

In an exemplary embodiment of the present application, L1 is a direct bond; a substituted or unsubstituted C6 to C60 arylene group; or a substituted or unsubstituted C2 to C60 heteroarylene group.

In another exemplary embodiment, L1 is a direct bond; a substituted or unsubstituted C6 to C40 arylene group; or a substituted or unsubstituted C2 to C40 heteroarylene group.

In still another exemplary embodiment, L1 is a direct bond; a substituted or unsubstituted C6 to C20 arylene group; or a substituted or unsubstituted C2 to C20 heteroarylene group.

In yet another exemplary embodiment, L1 is a direct bond; or a substituted or unsubstituted C6 to C20 arylene group.

In still yet another exemplary embodiment, L1 is a direct bond; or a C6 to C20 arylene group.

In a further exemplary embodiment, L1 is a direct bond; a substituted or unsubstituted phenylene group; a substituted or unsubstituted biphenylene group; a substituted or unsubstituted terphenylene group; or a substituted or unsubstituted quaterphenylene group.

In another further exemplary embodiment, L1 is a direct bond; a phenylene group; a biphenylene group; a terphenylene group; or a quaterphenylene group.

In an exemplary embodiment of the present application, Ar1 is a substituted or unsubstituted C1 to C60 alkyl group; a substituted or unsubstituted C6 to C60 aryl group; or a substituted or unsubstituted C2 to C60 heteroaryl group including O or S.

In an exemplary embodiment of the present application, Ar1 is a substituted or unsubstituted C6 to C60 aryl group; or a substituted or unsubstituted C2 to C60 heteroaryl group including O or S.

In an exemplary embodiment of the present application, Ar1 is a substituted or unsubstituted C6 to C40 aryl group; or a substituted or unsubstituted C2 to C40 heteroaryl group including O or S.

In an exemplary embodiment of the present application, Ar1 is a substituted or unsubstituted C6 to C30 aryl group; or a substituted or unsubstituted C2 to C30 heteroaryl group including O or S.

In an exemplary embodiment of the present application, Ar1 may be a substituted or unsubstituted phenyl group; a substituted or unsubstituted biphenyl group; a substituted or unsubstituted terphenyl group; a substituted or unsubstituted quaterphenyl group; a substituted or unsubstituted quinquephenyl group; a substituted or unsubstituted triphenylenyl group; a substituted or unsubstituted dibenzofuran group; a substituted or unsubstituted dibenzothiophene group; a substituted or unsubstituted dimethylfluorenyl group; a substituted or unsubstituted diphenylfluorenyl group; or a substituted or unsubstituted spirobifluorenyl group.

In an exemplary embodiment of the present application, Ar1 may be a phenyl group; a biphenyl group unsubstituted or substituted with a phenyl group; a terphenyl group unsubstituted or substituted with a phenyl group; a quaterphenyl group; a quinquephenyl group; a triphenylenyl group; a dibenzofuran group unsubstituted or substituted with a phenyl group or a biphenyl group; a dibenzothiophene group unsubstituted or substituted with a phenyl group or a biphenyl group; a dimethylfluorenyl group; a diphenyl fluorenyl group; or a spirobifluorenyl group.

In an exemplary embodiment of the present application, provided is a heterocyclic compound in which Chemical Formula 1 is represented by any one of the following compounds.

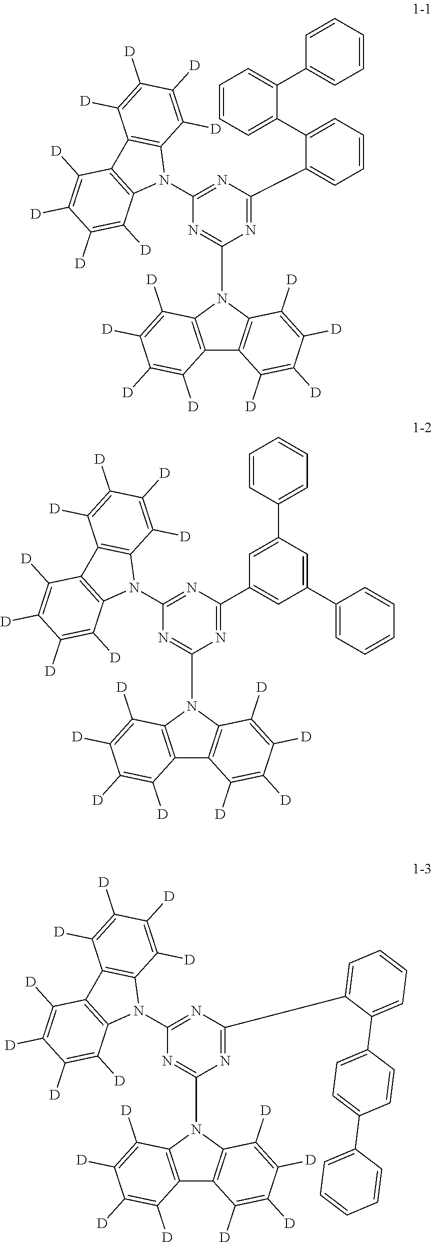

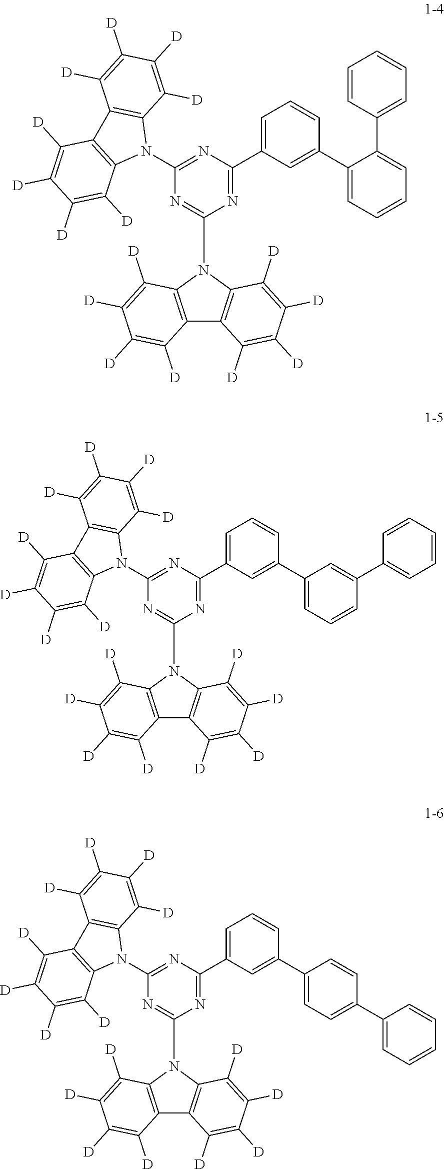

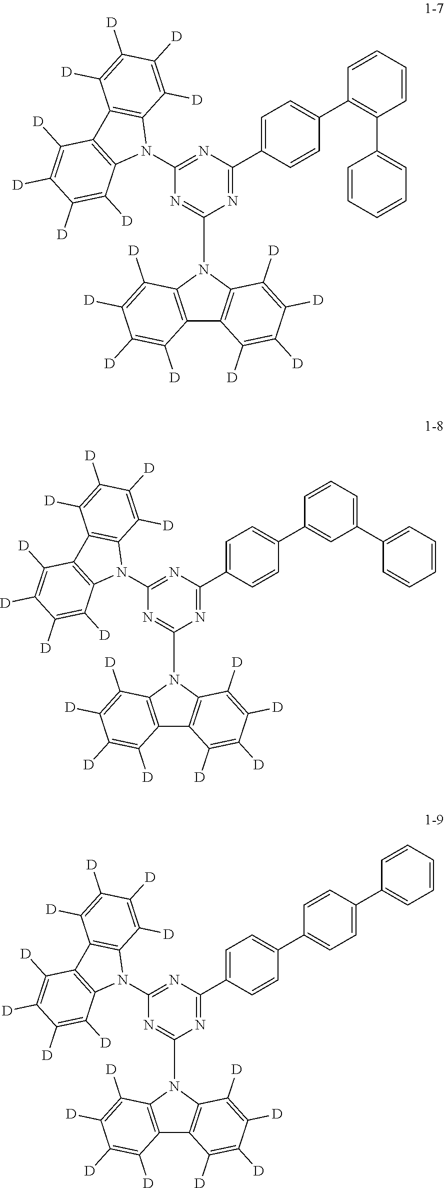

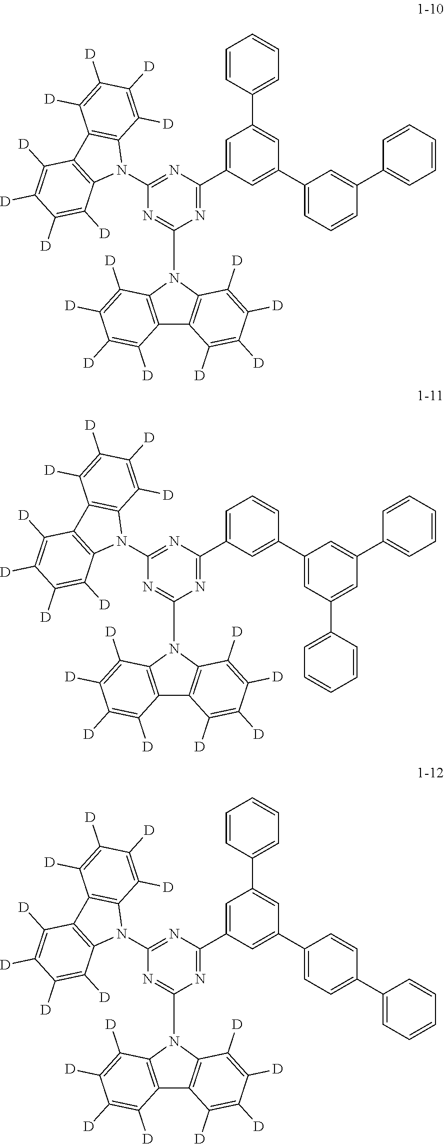

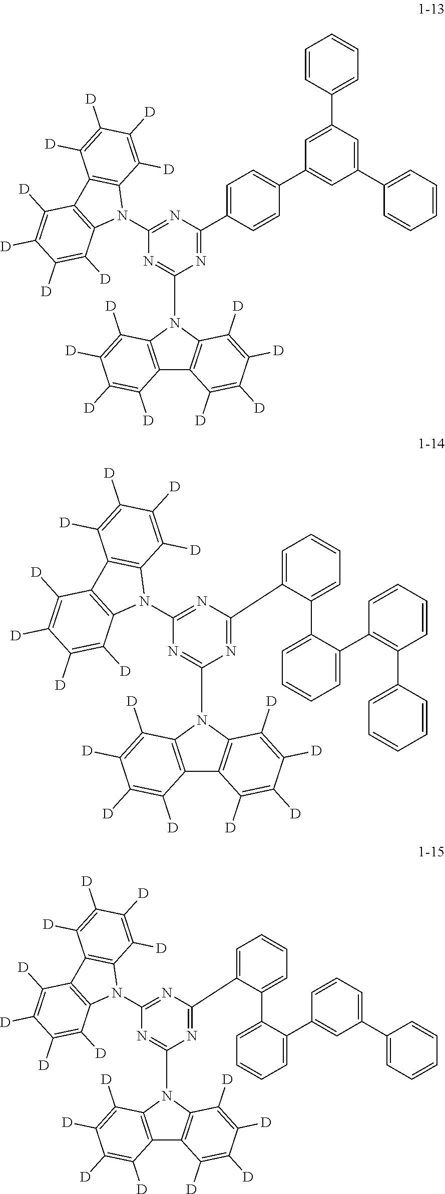

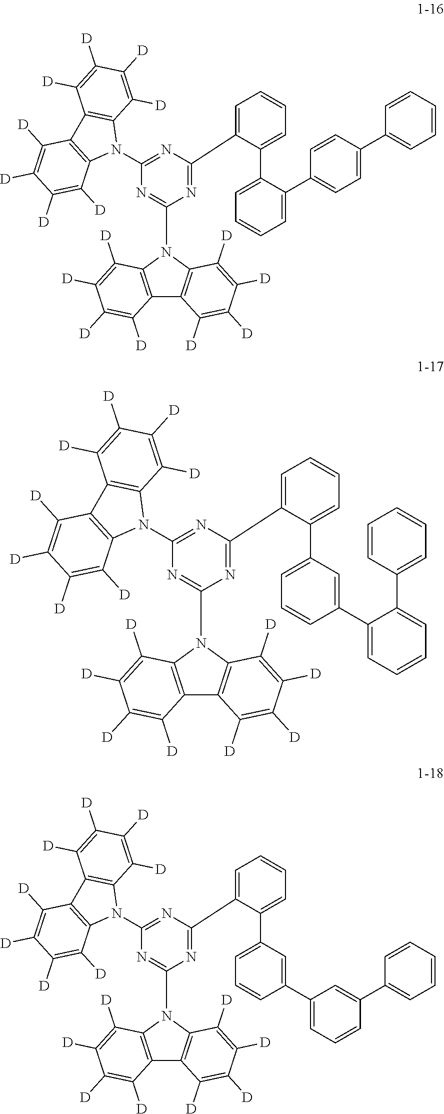

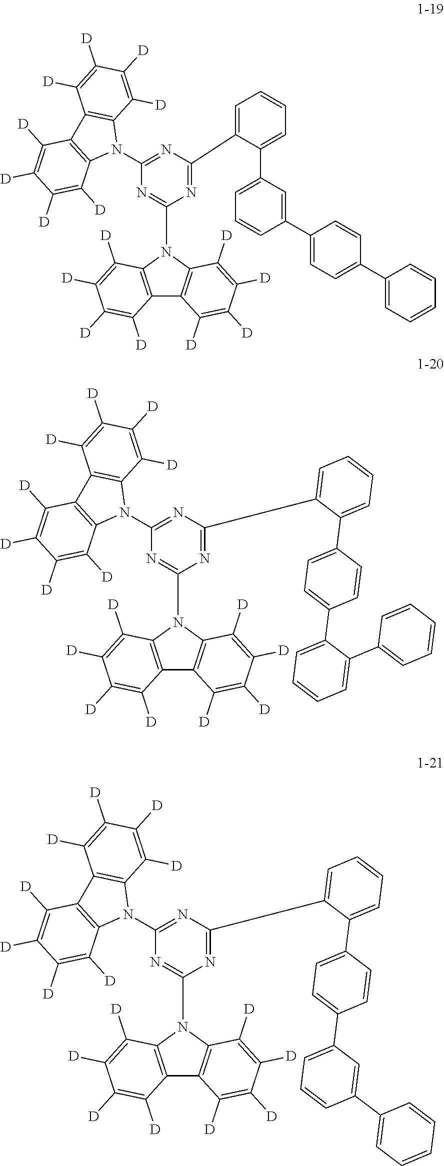

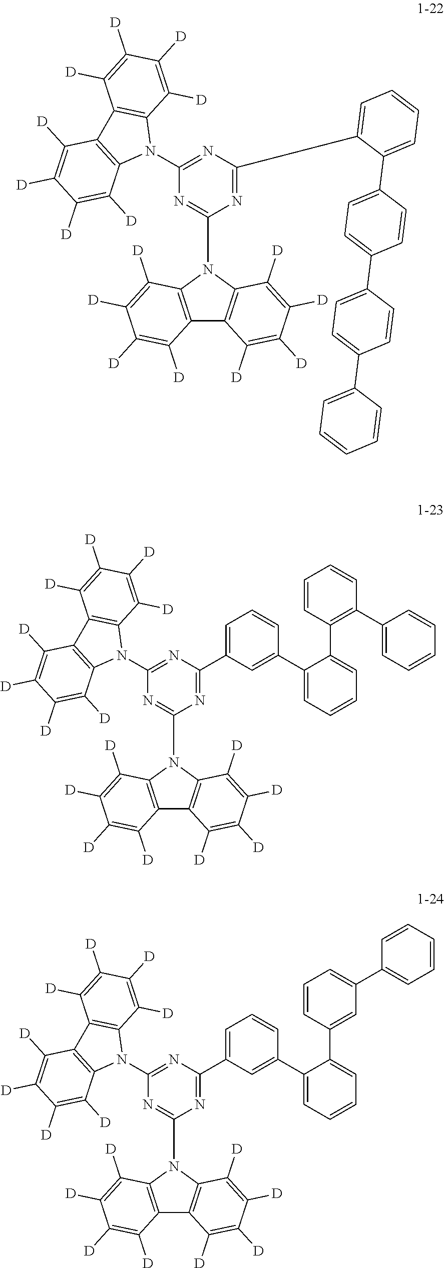

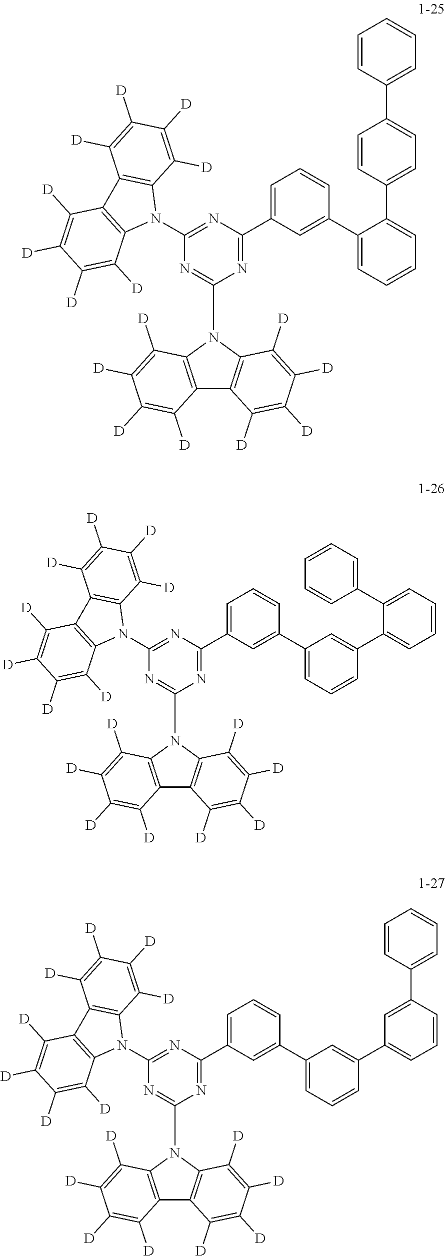

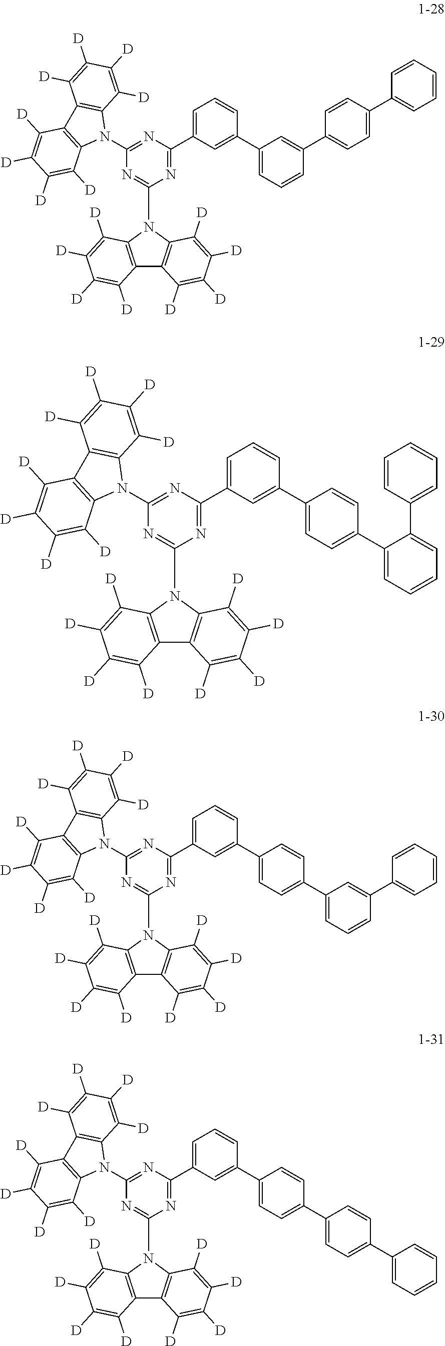

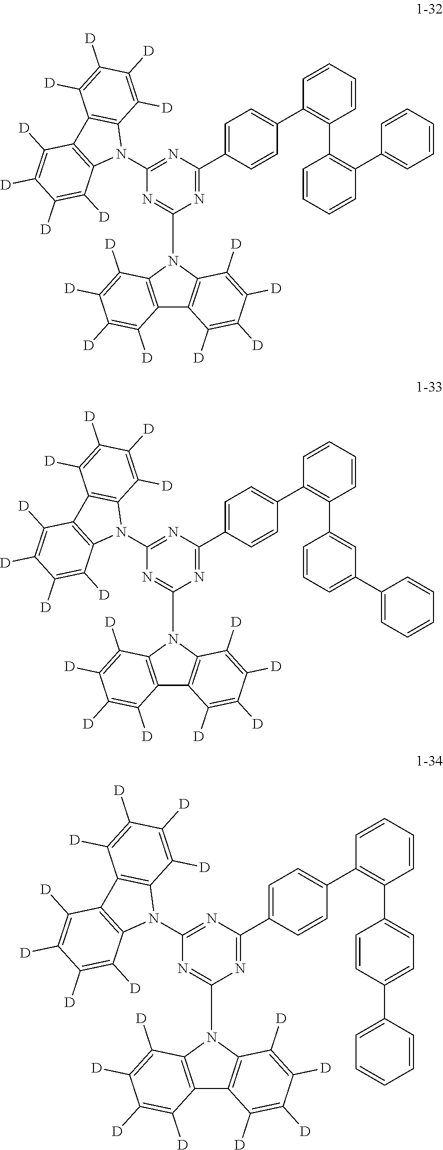

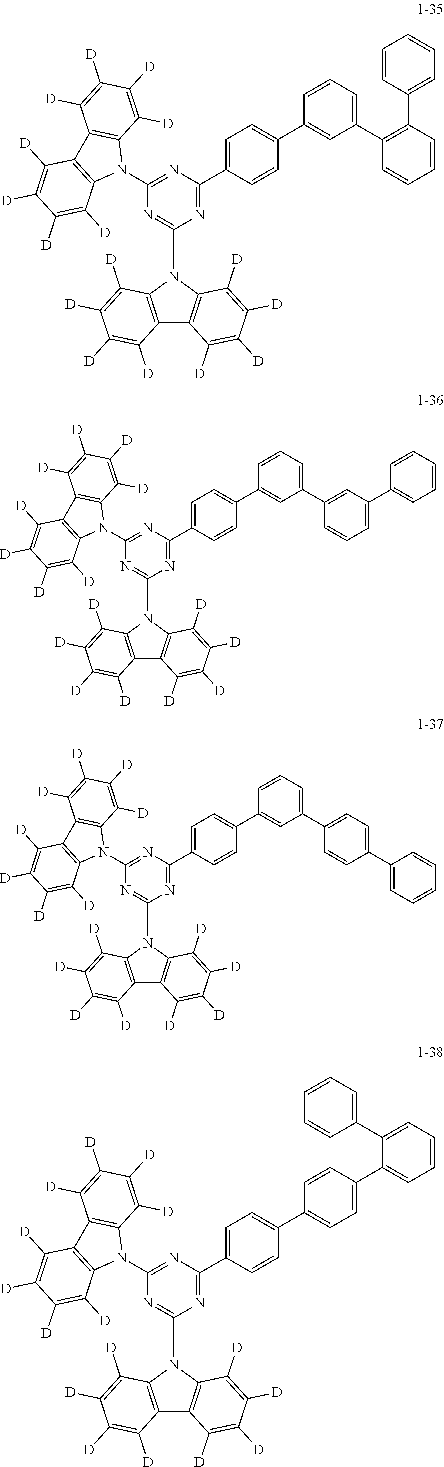

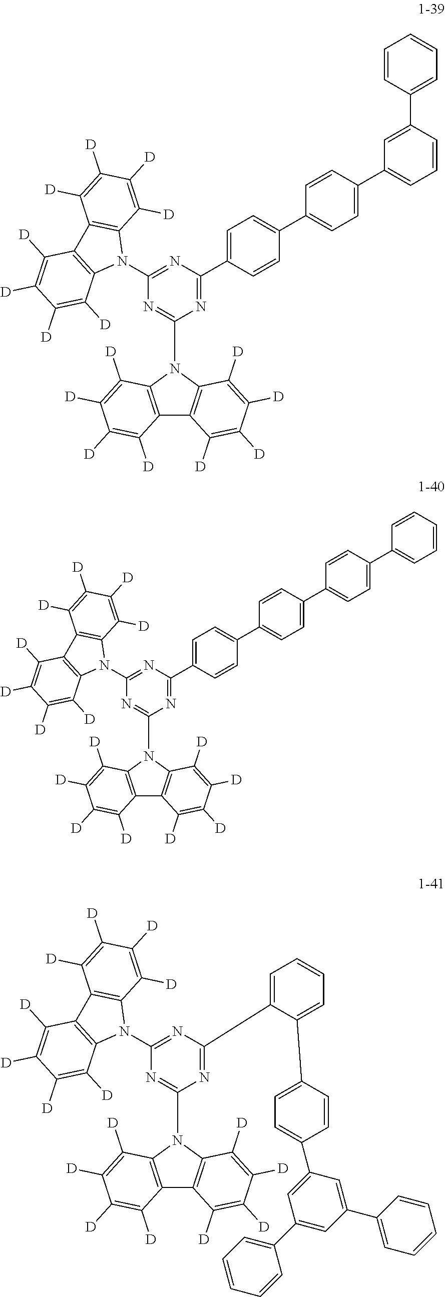

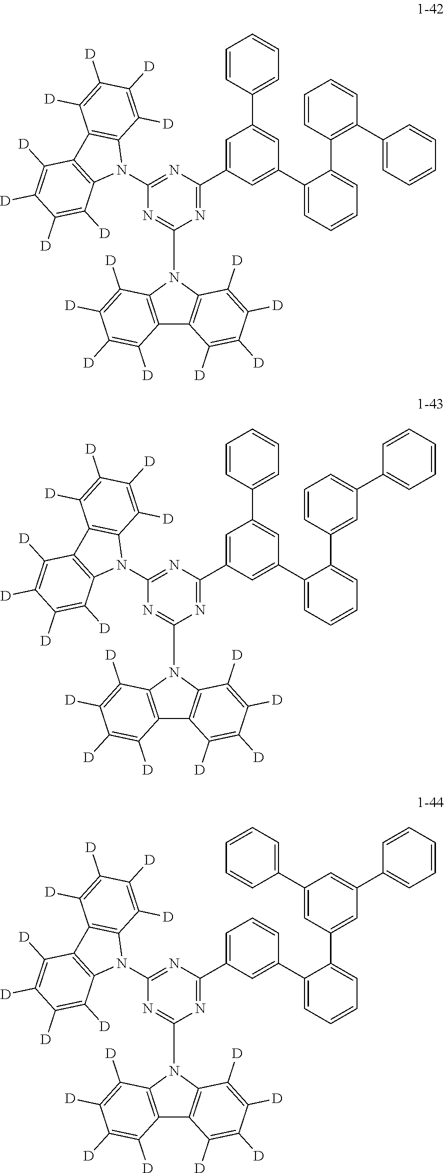

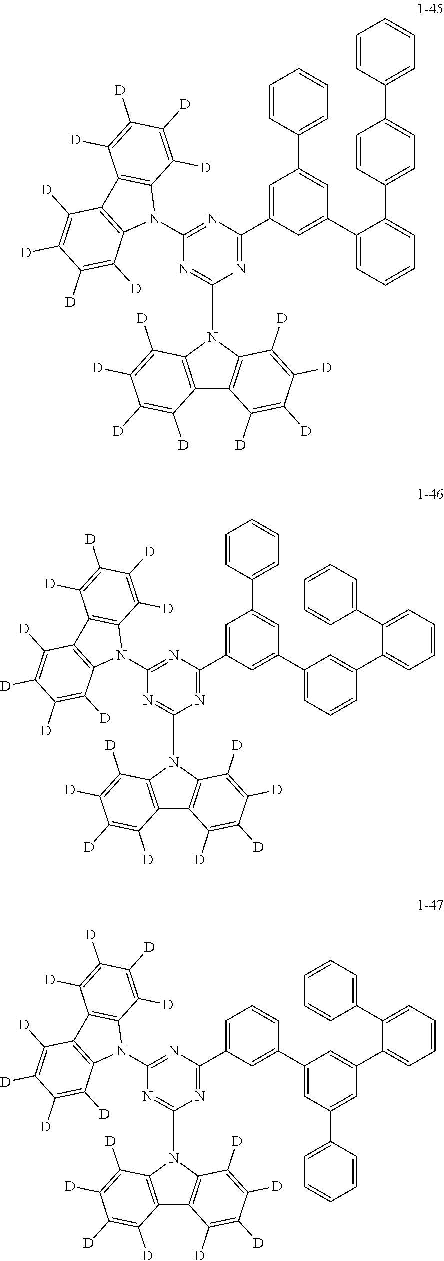

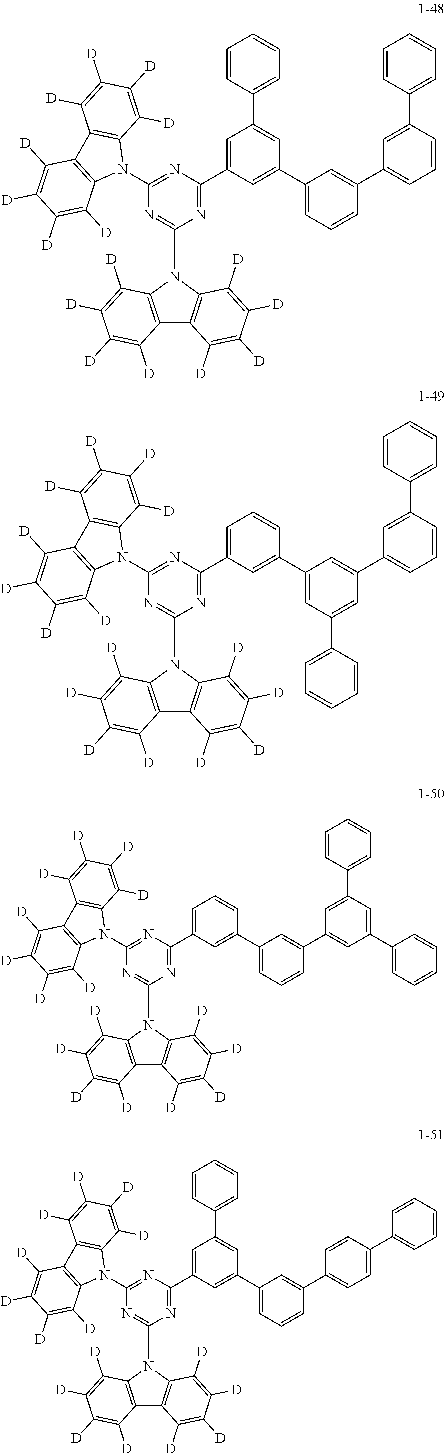

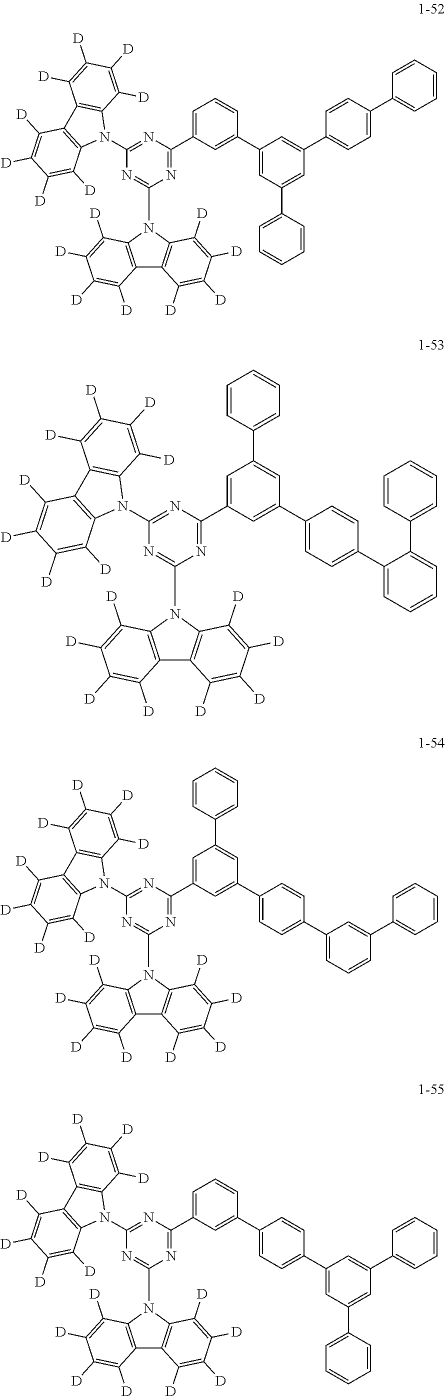

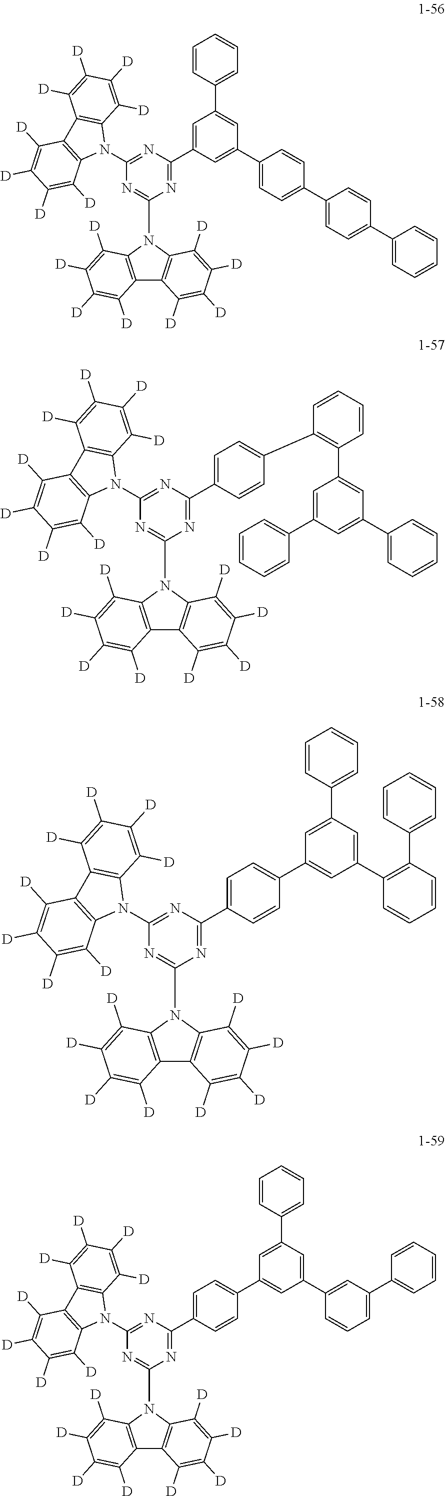

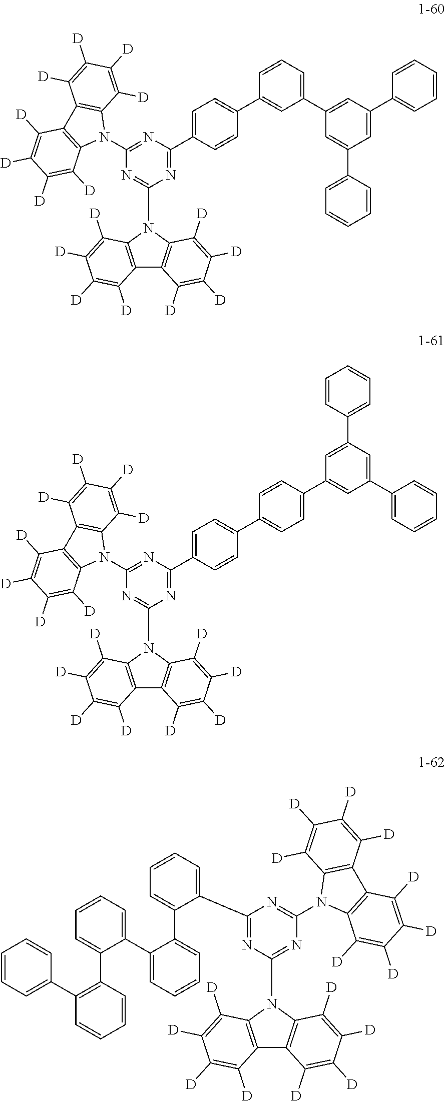

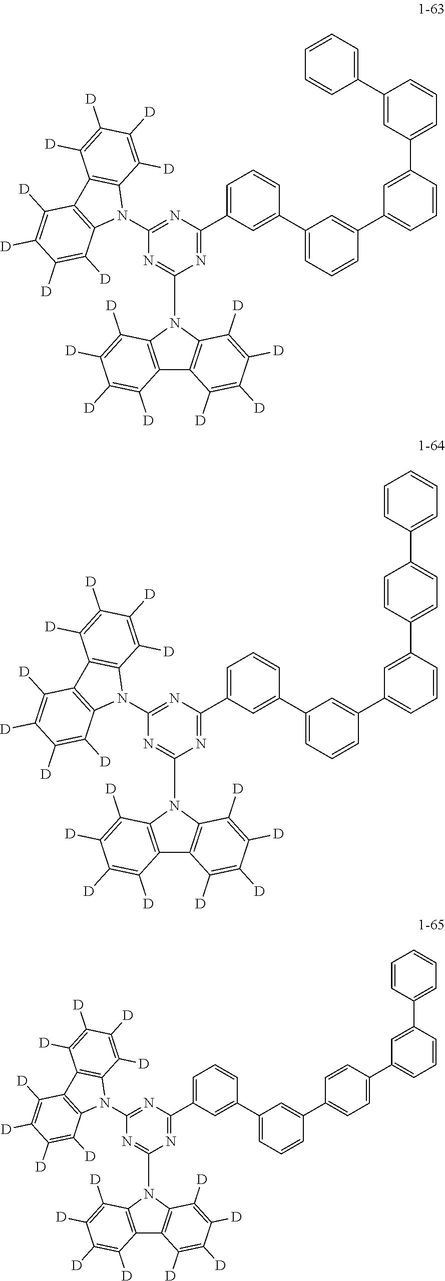

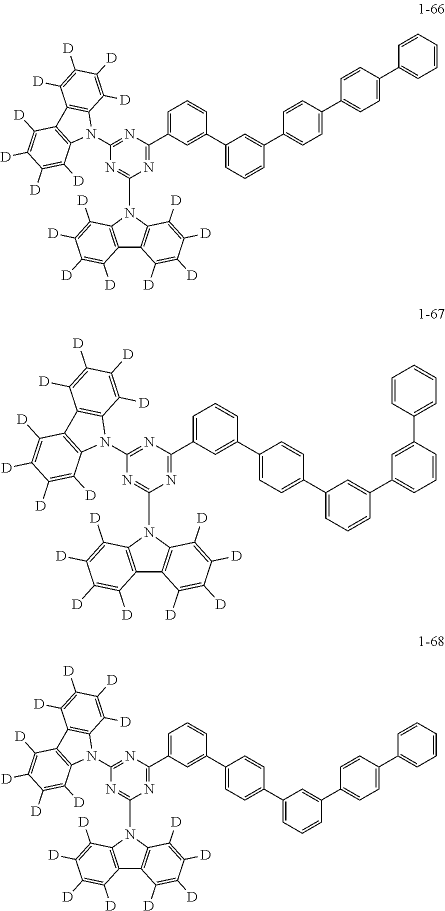

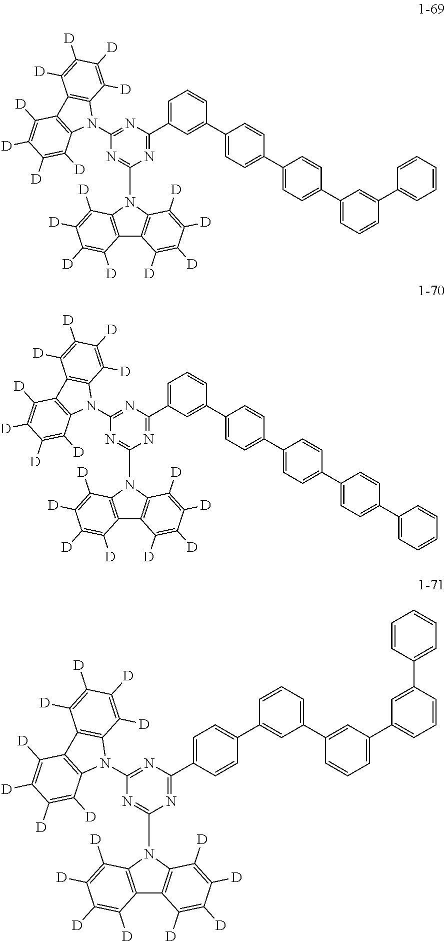

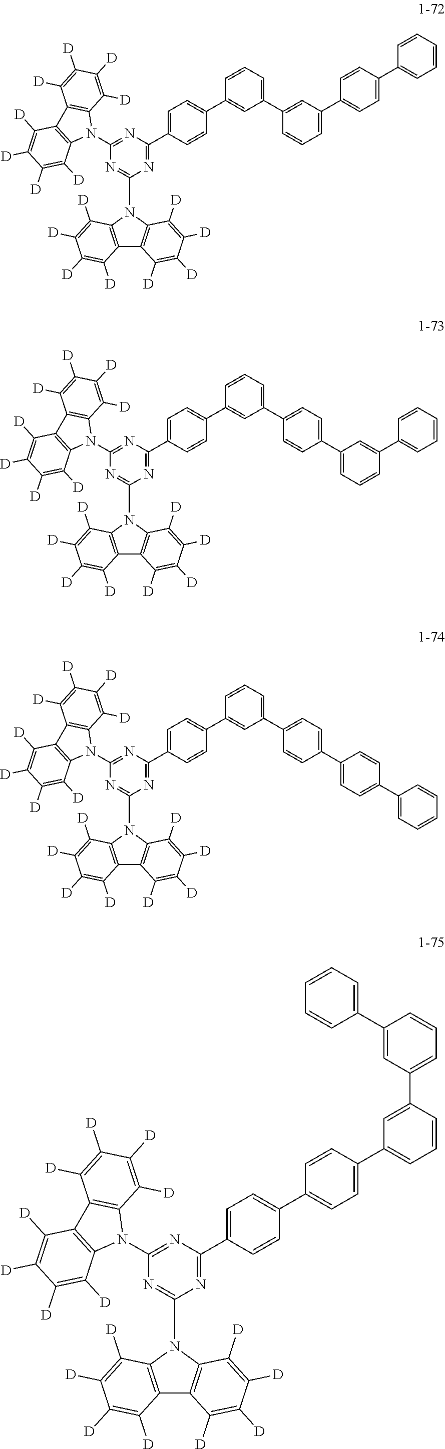

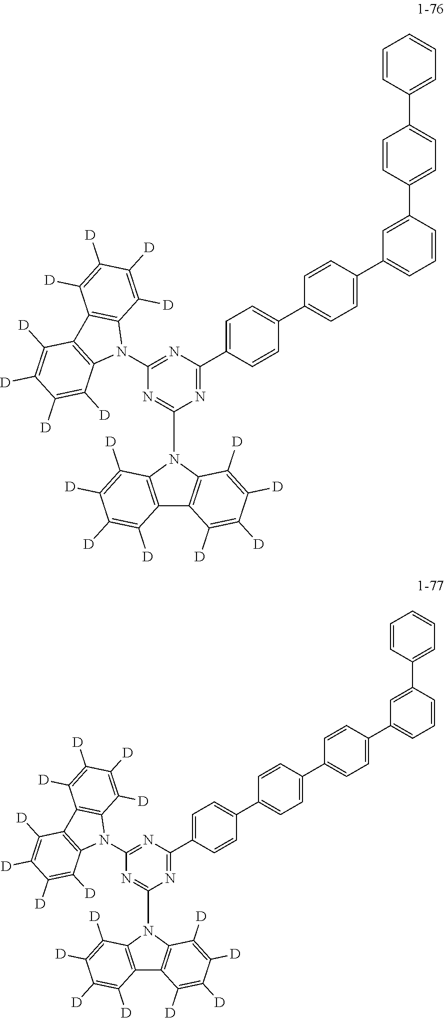 [SMILES: [2H]c1c([2H])c([2H])c2c(c1[2H])c1c([2H])c([2H])c([2H])c([2H])c1n2-c1nc(-c2ccc(-c3ccc(-c4cccc(-c5ccc(-c6ccccc6)cc5)c4)cc3)cc2)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2ccc(-c3ccc(-c4ccc(-c5cccc(-c6ccccc6)c5)cc4)cc3)cc2)nc(-n2c3c([2H])c([2H])c([2H])c([2H])c3c3c([2H])c([2H])c([2H])c([2H])c32)n1]

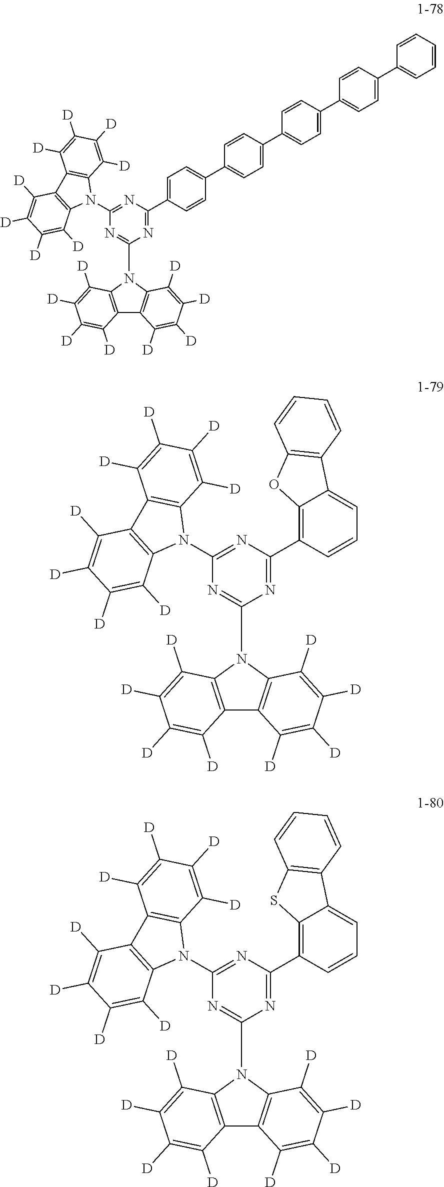

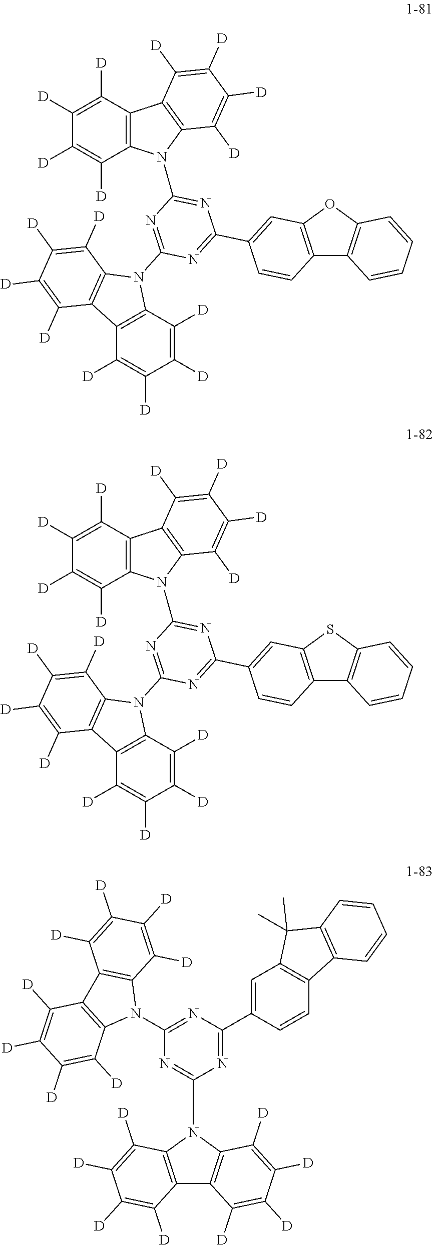

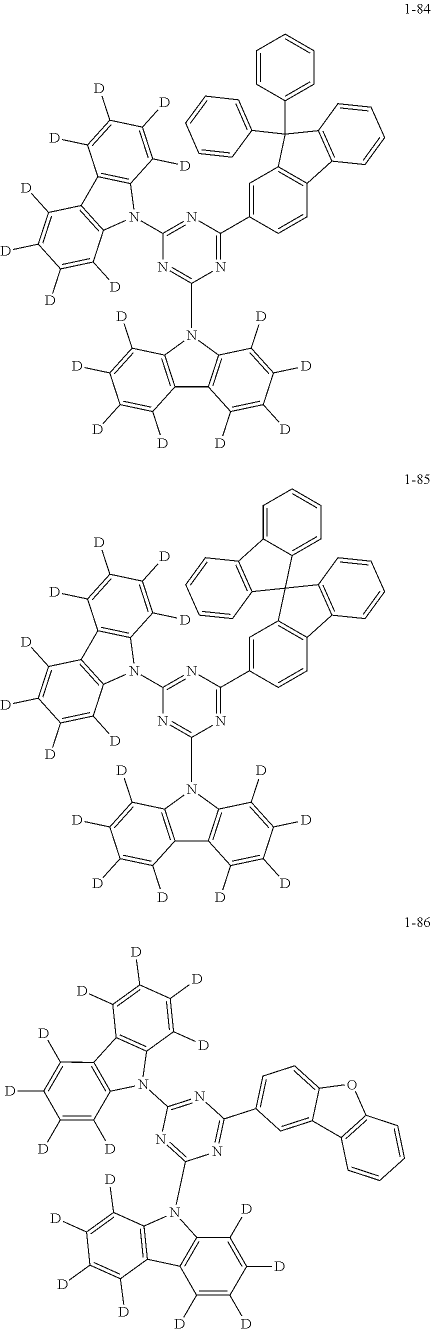

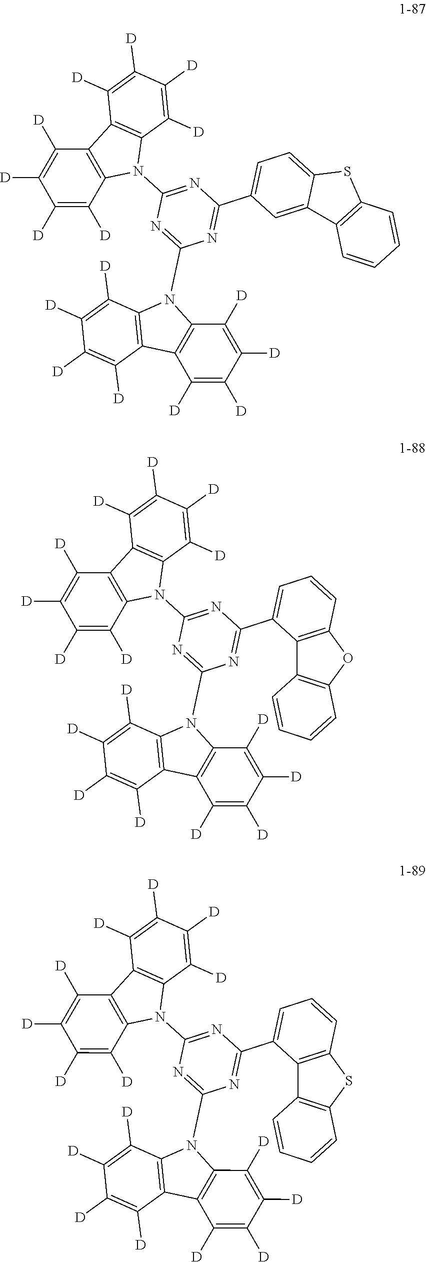

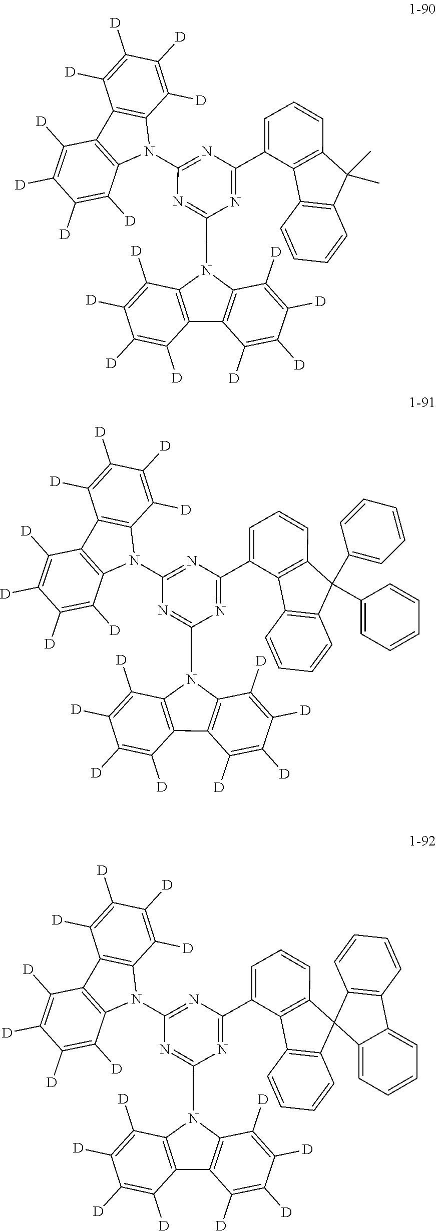

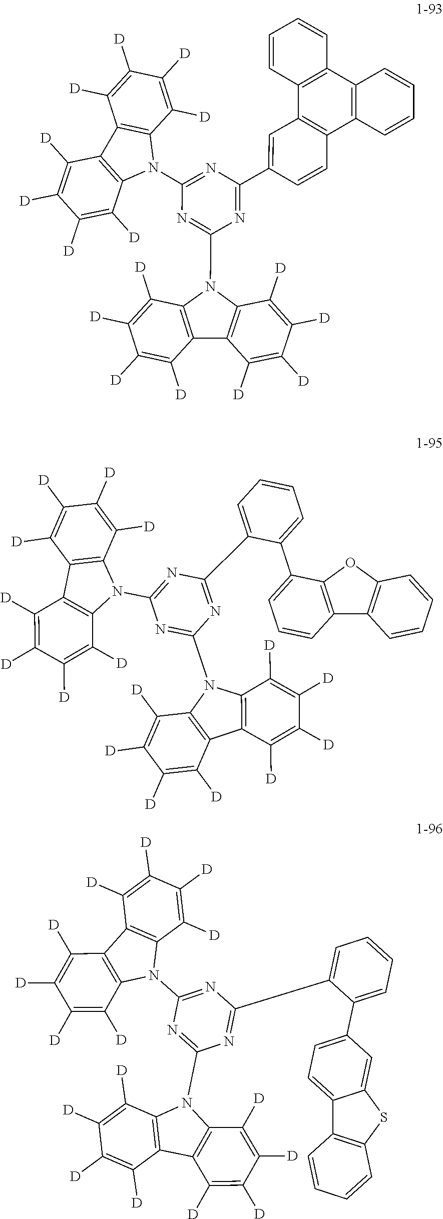

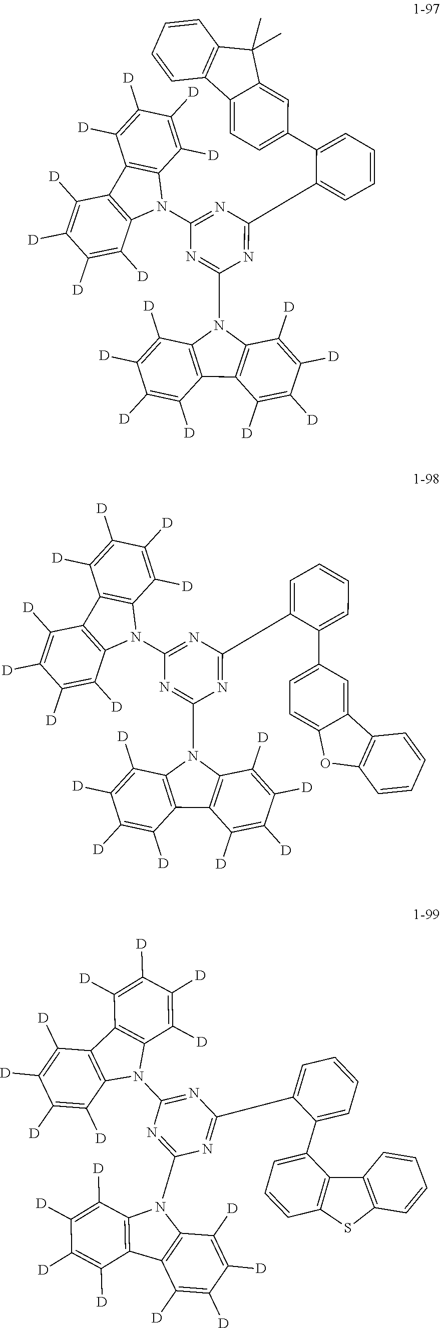

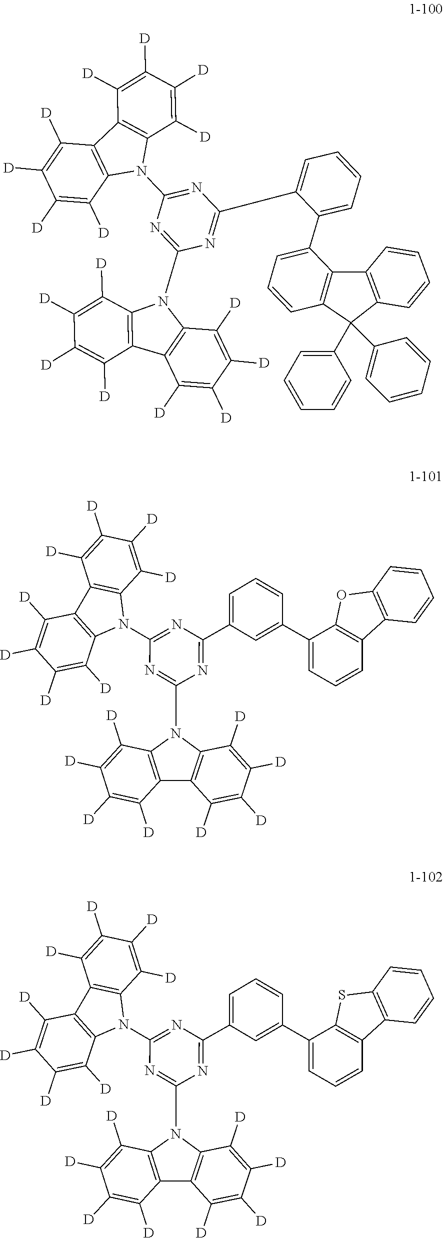

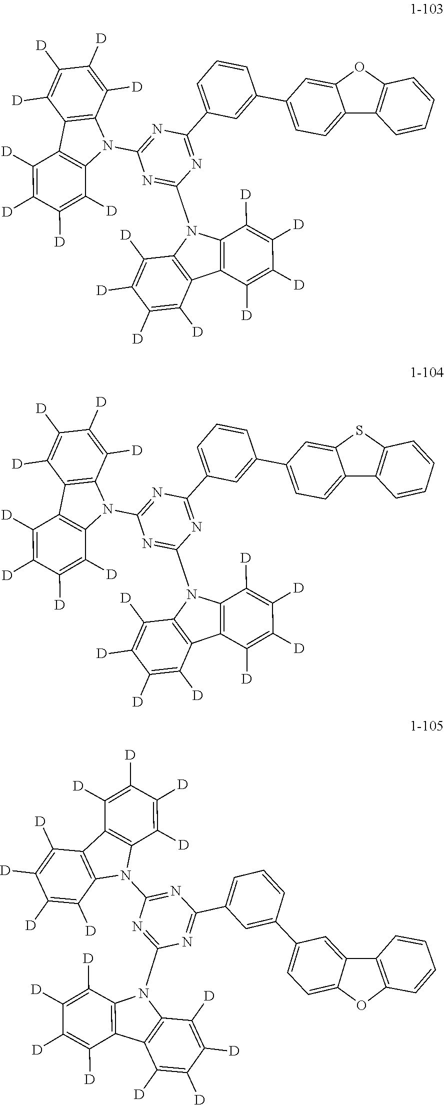

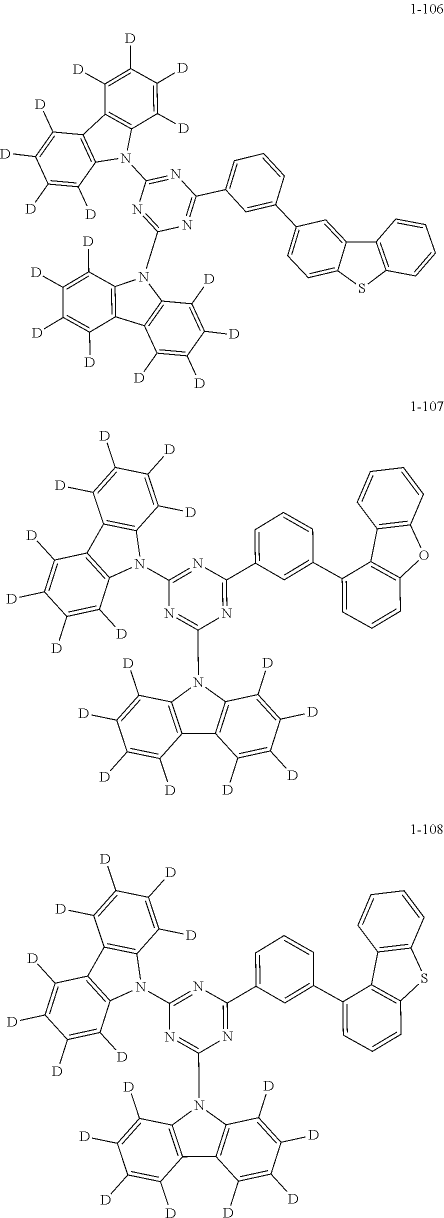

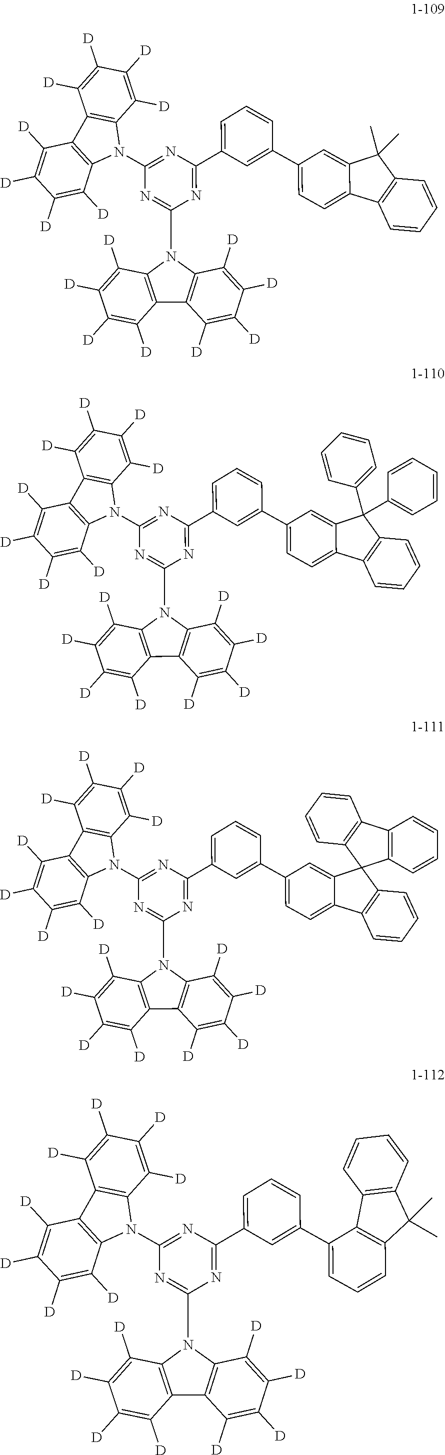

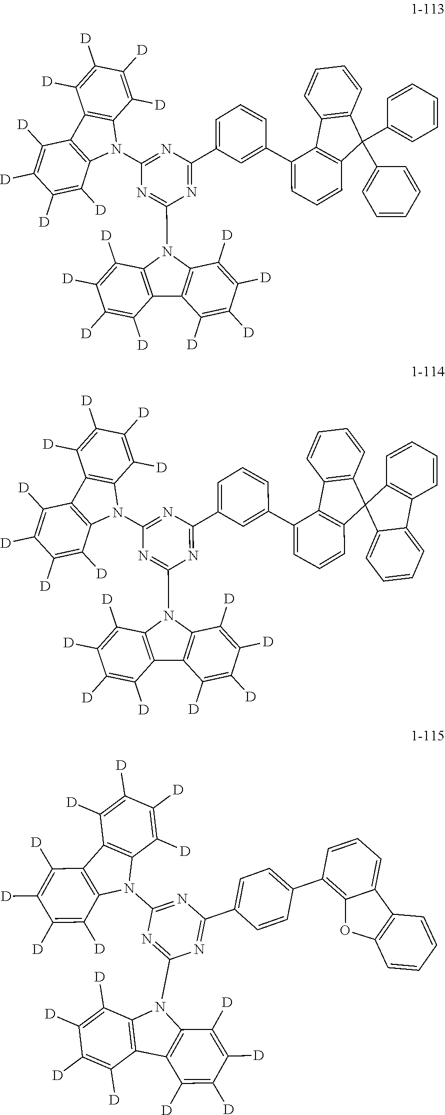

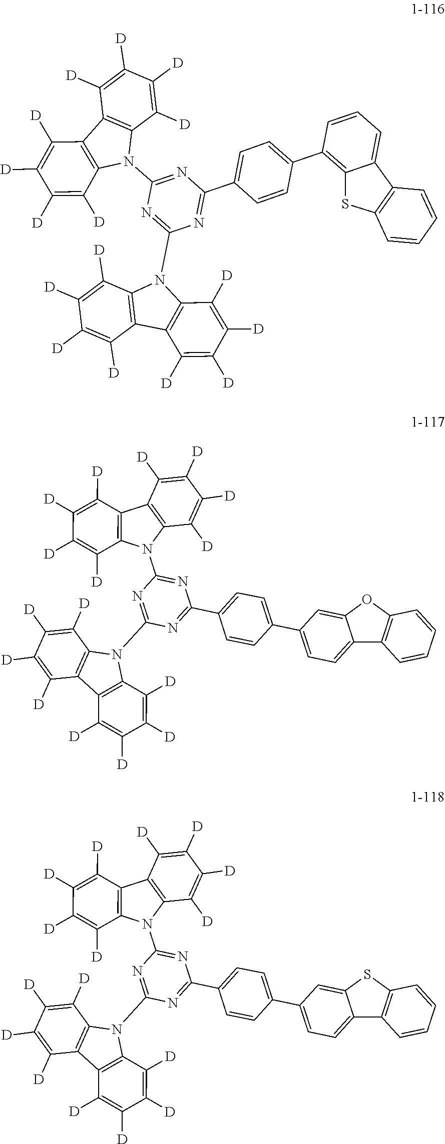

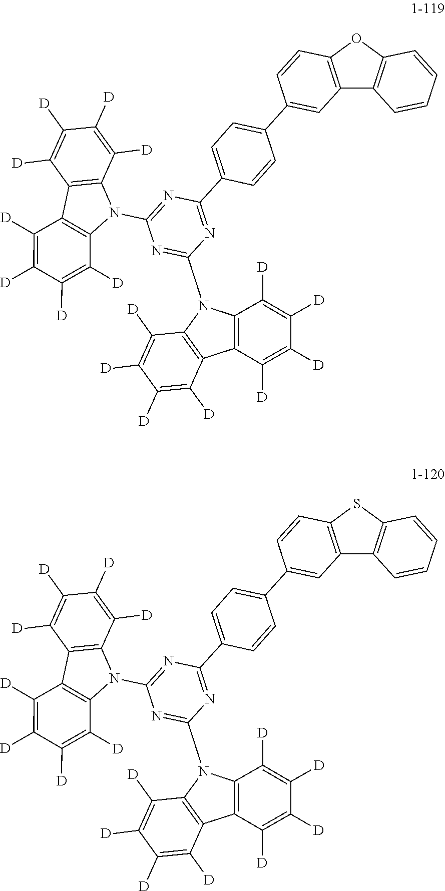 [SMILES: [2H]c1c([2H])c([2H])c2c(c1[2H])c1c([2H])c([2H])c([2H])c([2H])c1n2-c1nc(-c2ccc(-c3ccc4oc5ccccc5c4c3)cc2)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2ccc(-c3ccc4sc5ccccc5c4c3)cc2)nc(-n2c3c([2H])c([2H])c([2H])c([2H])c3c3c([2H])c([2H])c([2H])c([2H])c32)n1]

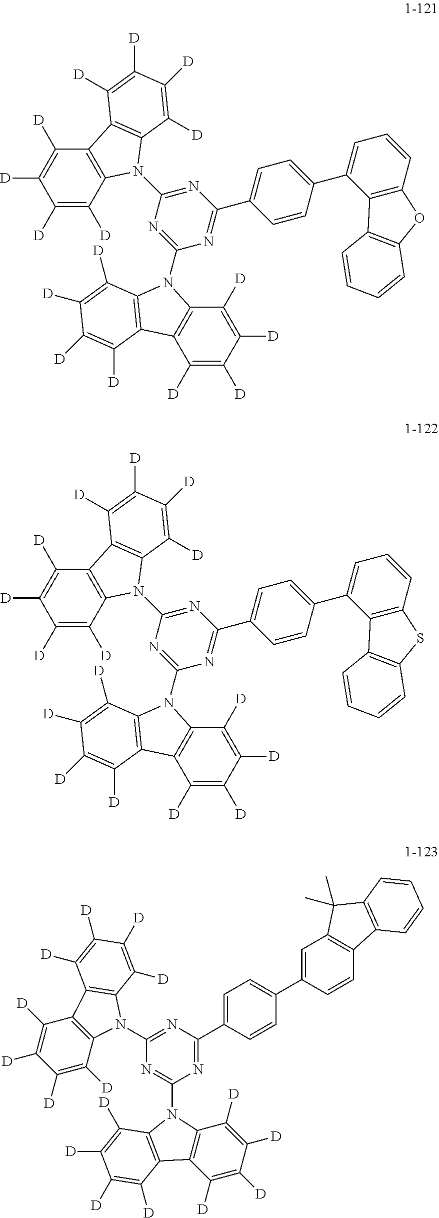

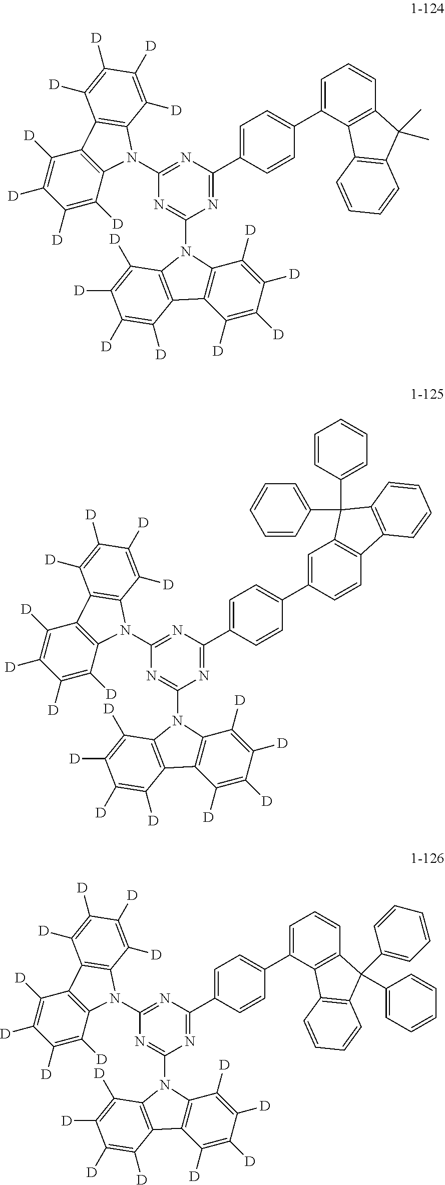

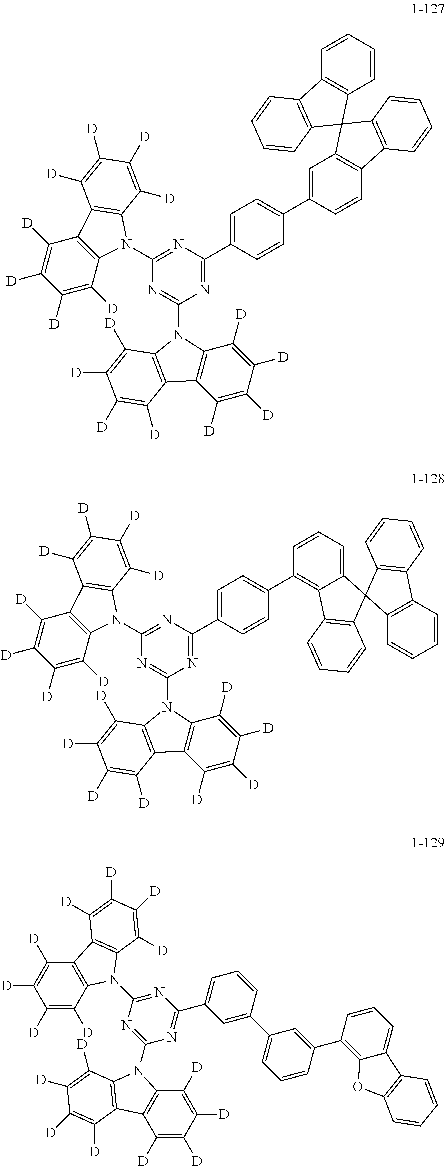

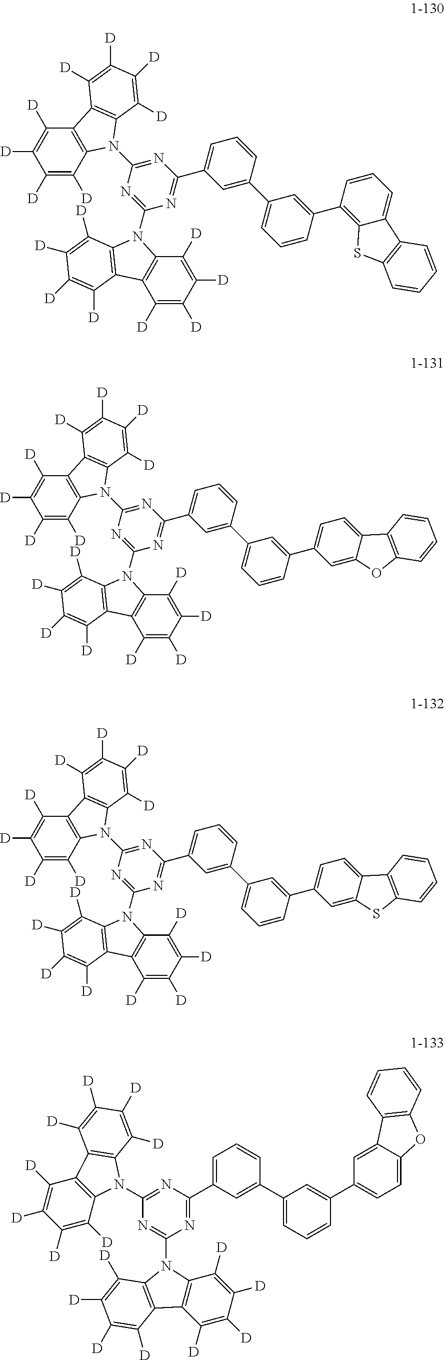

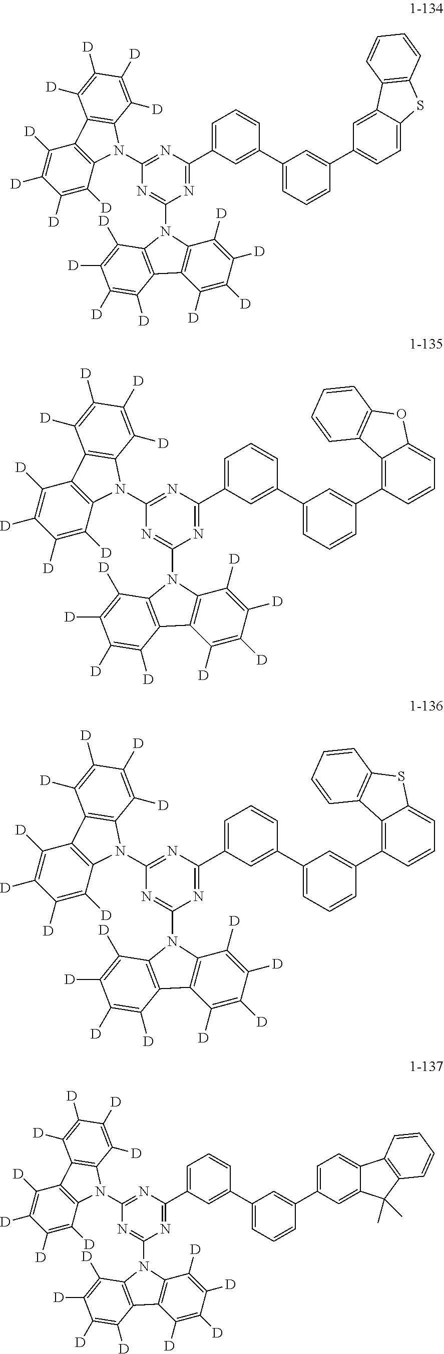

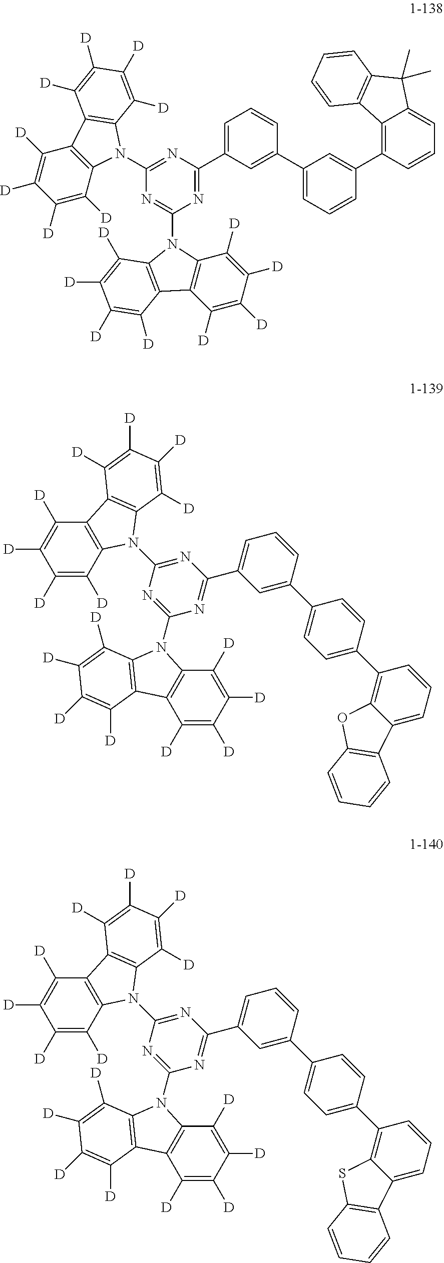

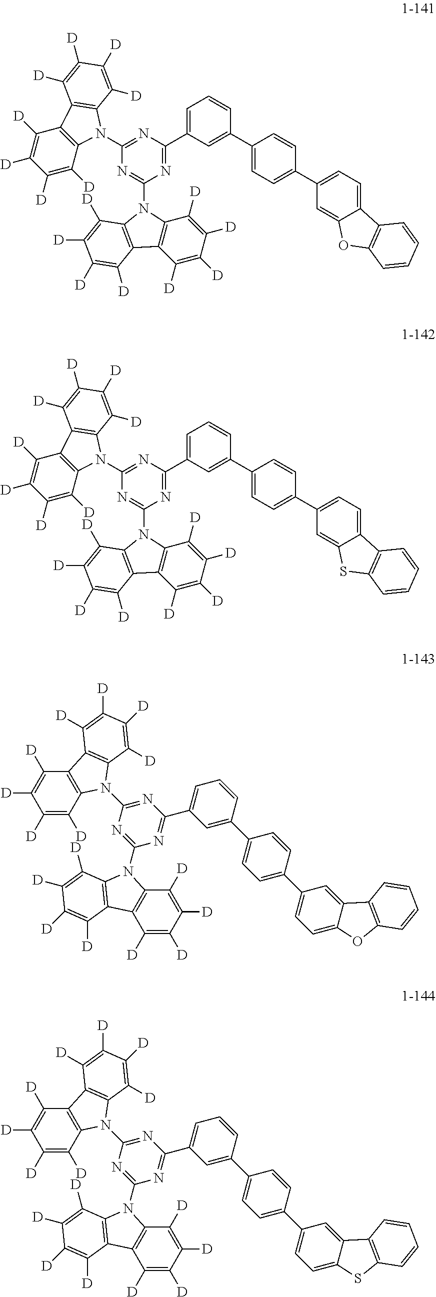

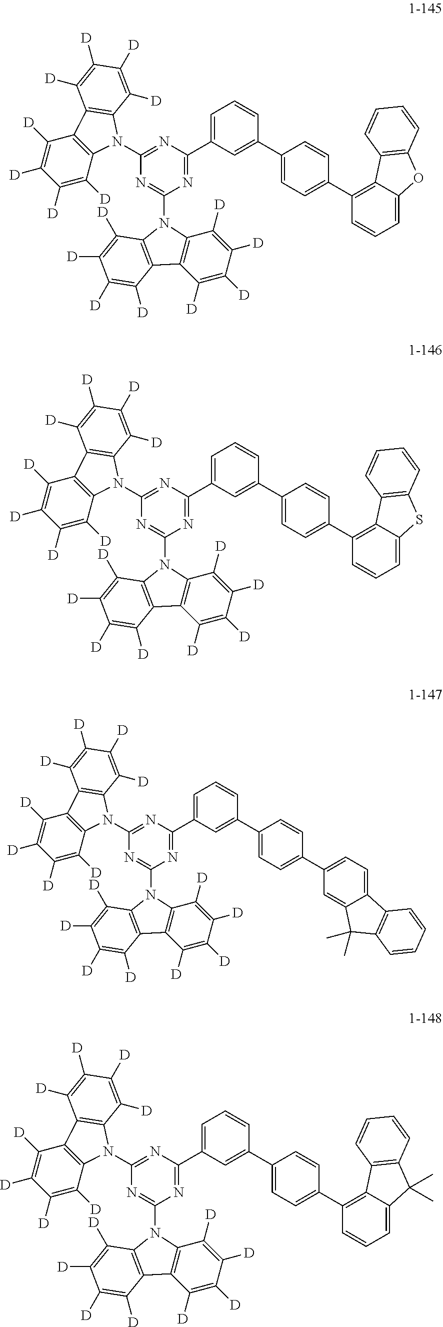

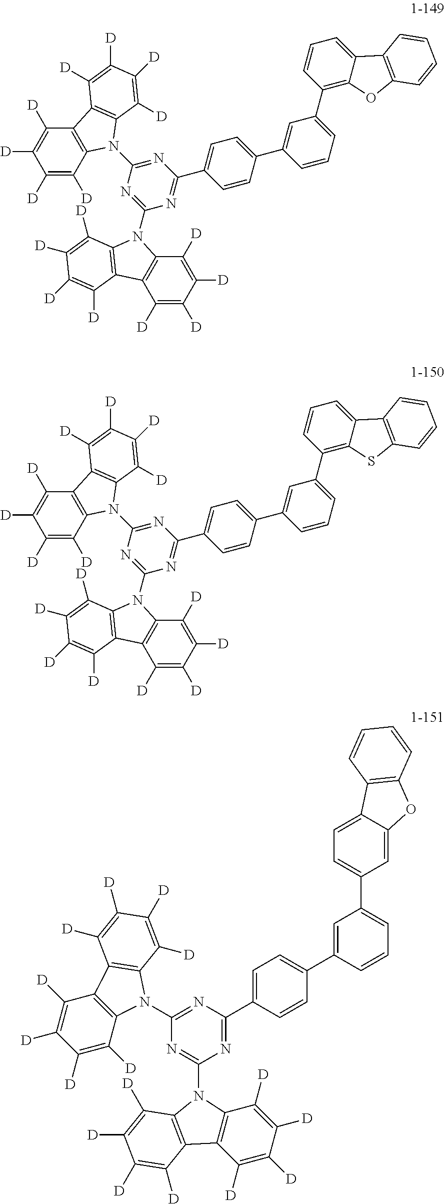

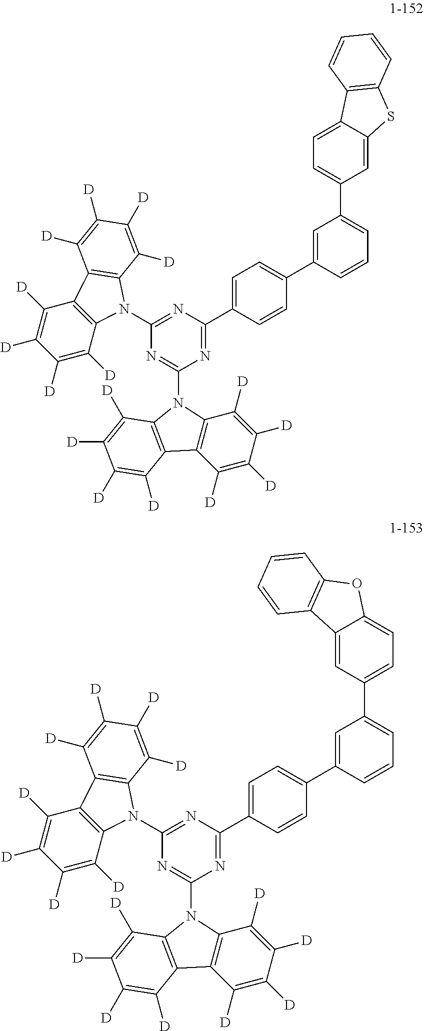 [SMILES: [2H]c1c([2H])c([2H])c2c(c1[2H])c1c([2H])c([2H])c([2H])c([2H])c1n2-c1nc(-c2ccc(-c3cccc(-c4ccc5c(c4)sc4ccccc45)c3)cc2)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2ccc(-c3cccc(-c4ccc5oc6ccccc6c5c4)c3)cc2)nc(-n2c3c([2H])c([2H])c([2H])c([2H])c3c3c([2H])c([2H])c([2H])c([2H])c32)n1]

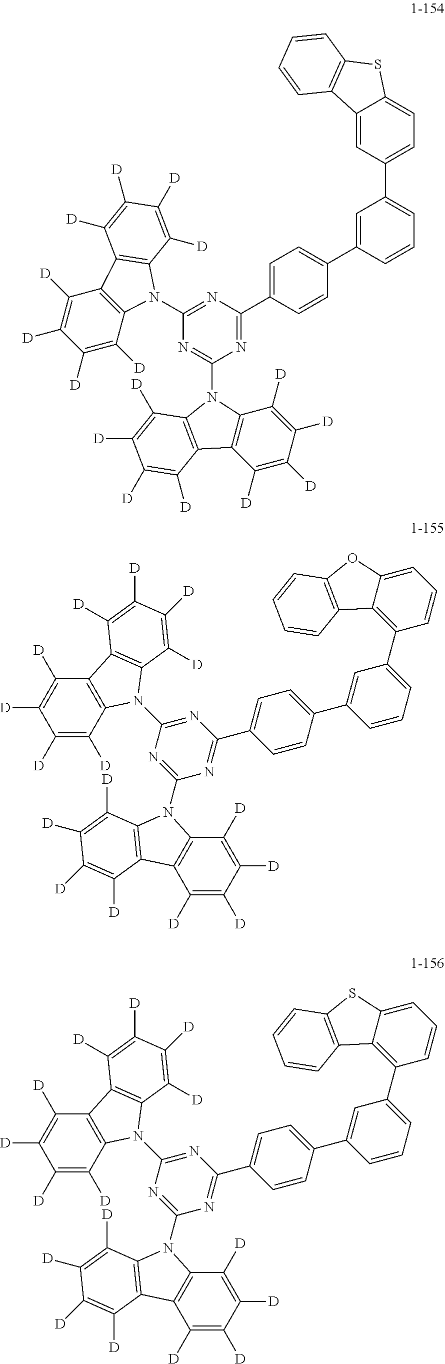

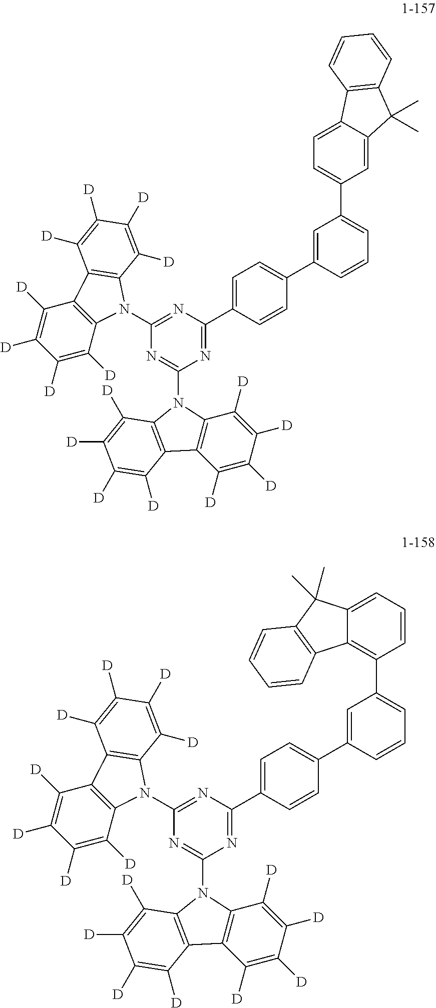 [SMILES: [2H]c1c([2H])c([2H])c2c(c1[2H])c1c([2H])c([2H])c([2H])c([2H])c1n2-c1nc(-c2ccc(-c3cccc(-c4ccc5c(c4)C(C)(C)c4ccccc4-5)c3)cc2)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2ccc(-c3cccc(-c4cccc5c4-c4ccccc4C5(C)C)c3)cc2)nc(-n2c3c([2H])c([2H])c([2H])c([2H])c3c3c([2H])c([2H])c([2H])c([2H])c32)n1]

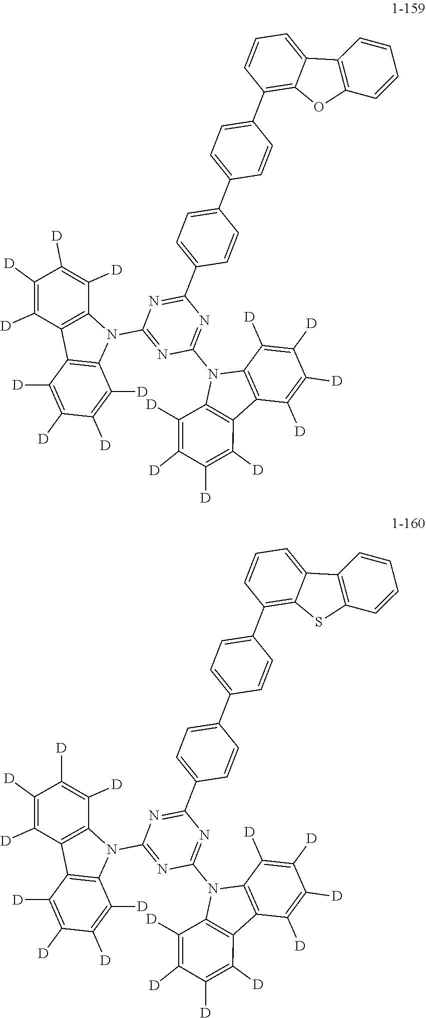 [SMILES: [2H]c1c([2H])c([2H])c2c(c1[2H])c1c([2H])c([2H])c([2H])c([2H])c1n2-c1nc(-c2ccc(-c3ccc(-c4cccc5c4oc4ccccc45)cc3)cc2)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2ccc(-c3ccc(-c4cccc5c4sc4ccccc45)cc3)cc2)nc(-n2c3c([2H])c([2H])c([2H])c([2H])c3c3c([2H])c([2H])c([2H])c([2H])c32)n1]

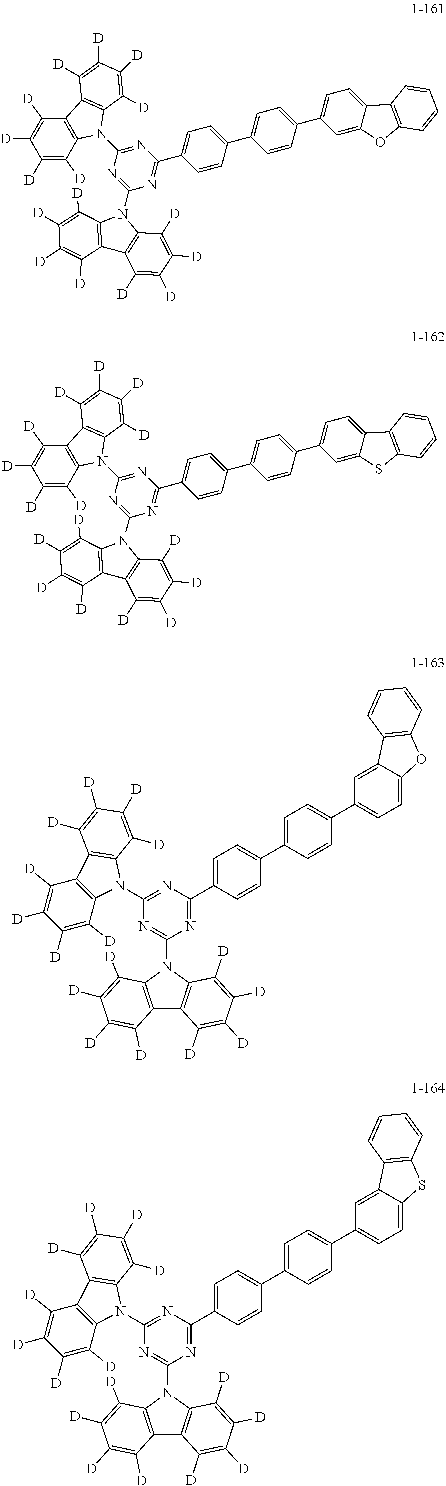

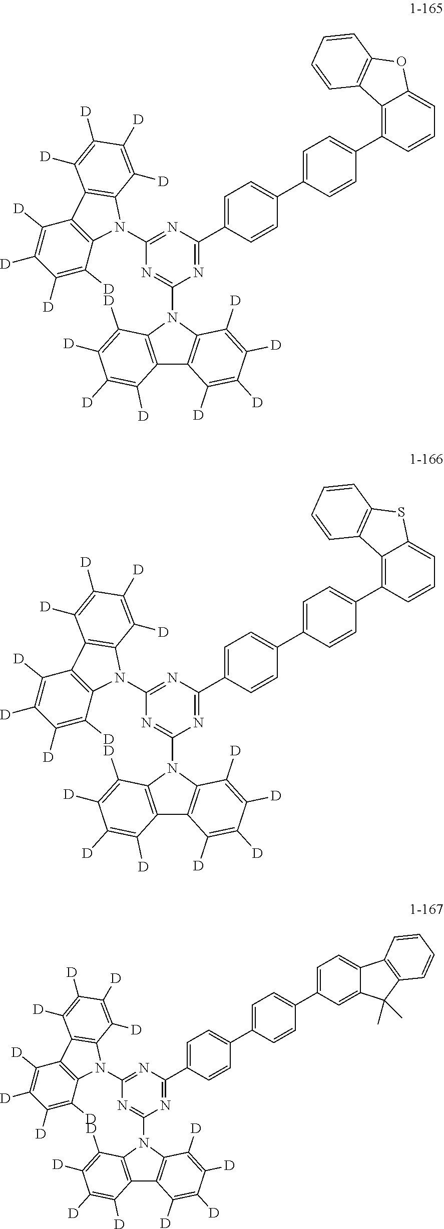

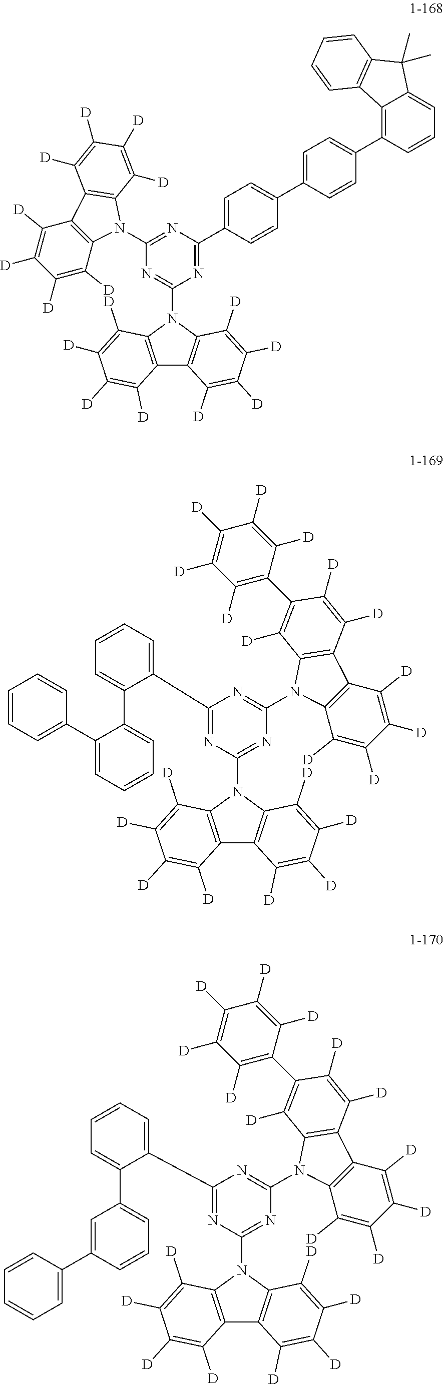

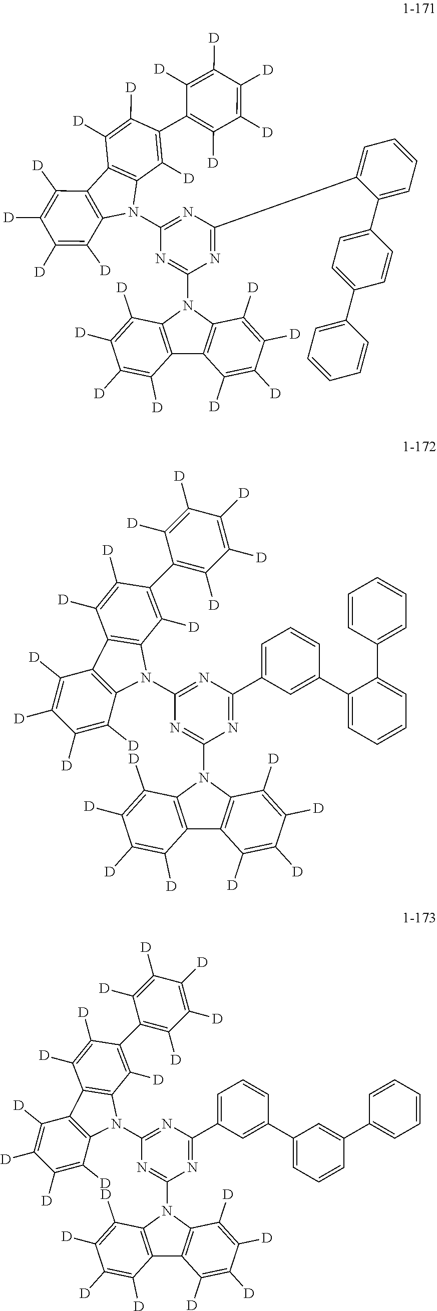

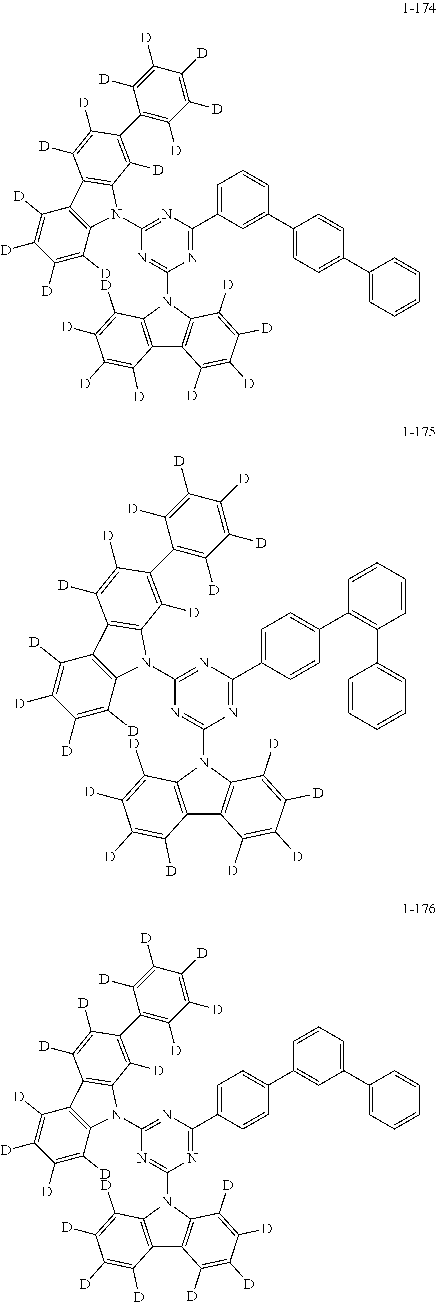

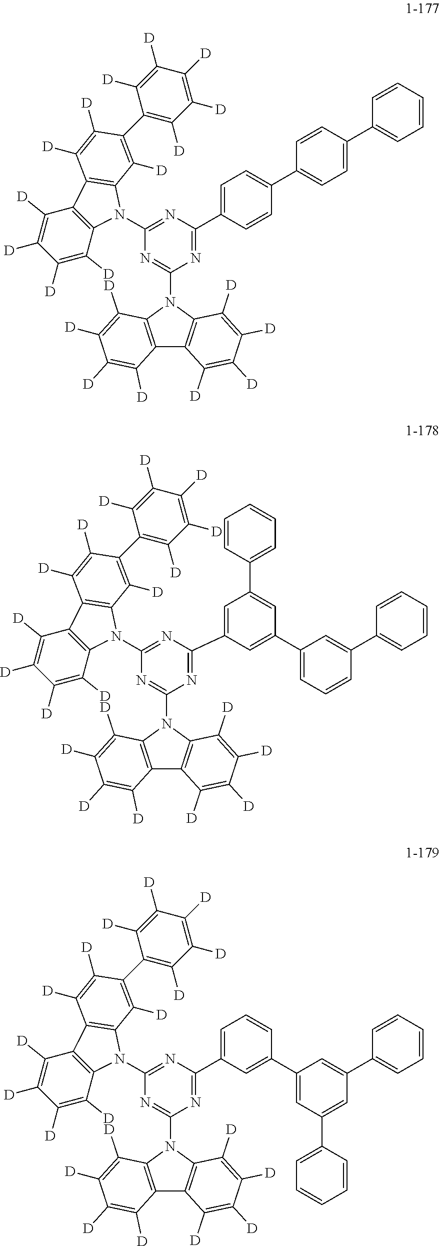

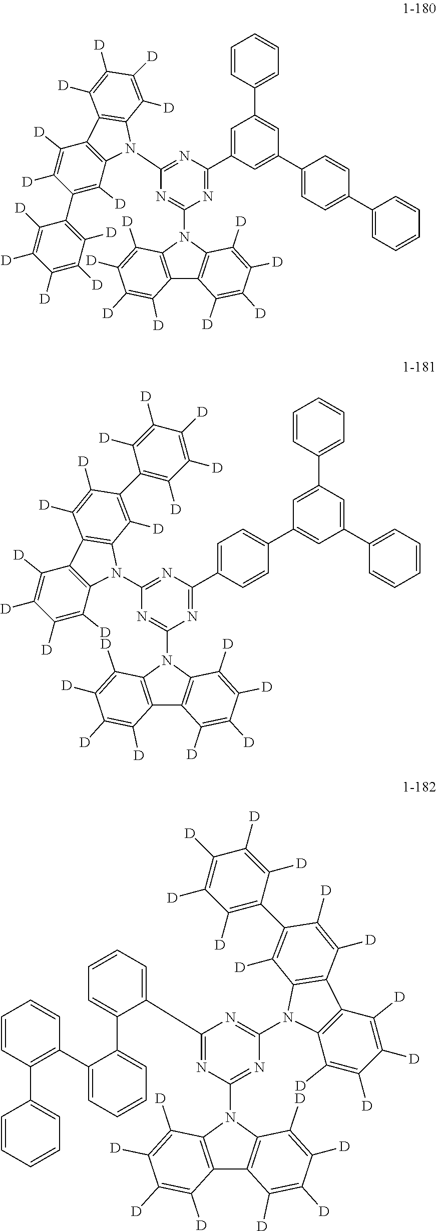

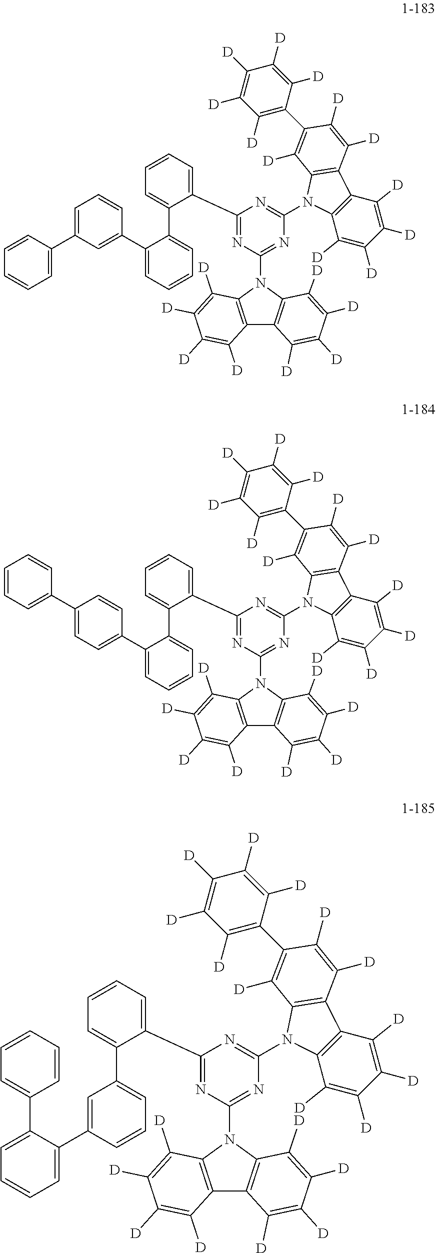

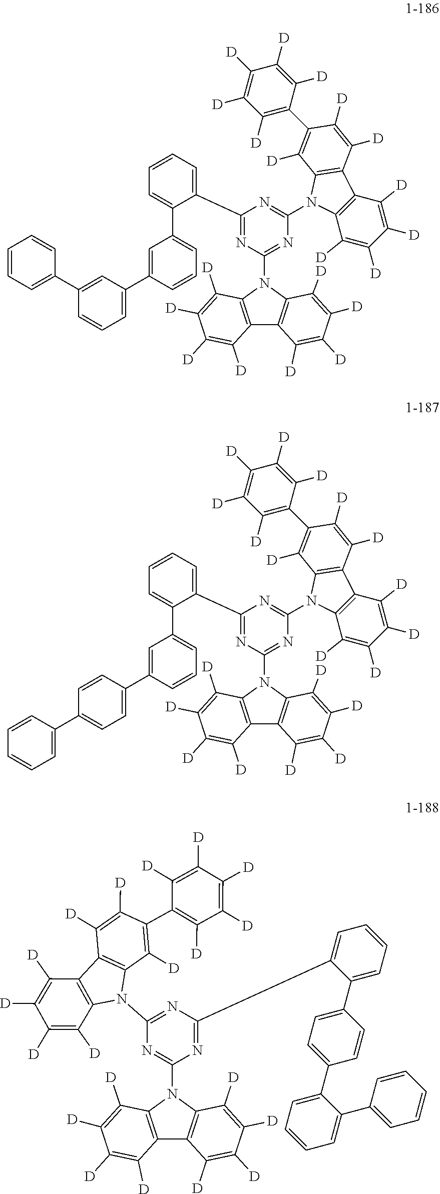

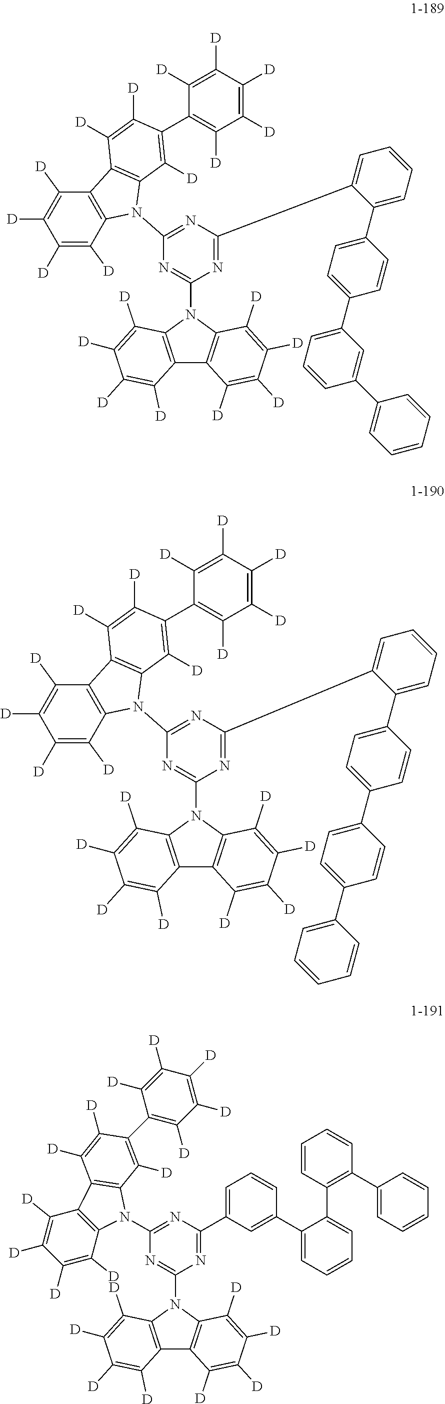

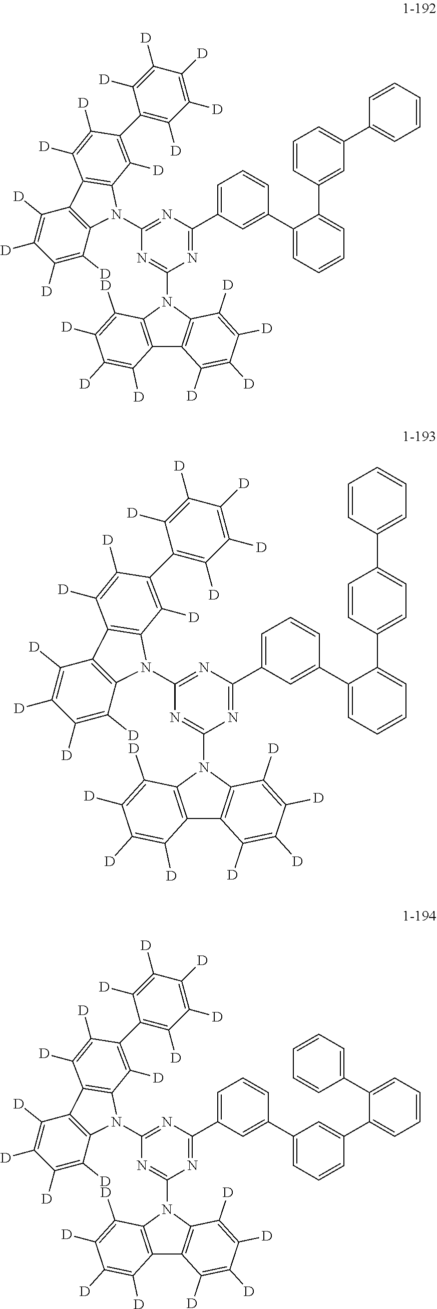

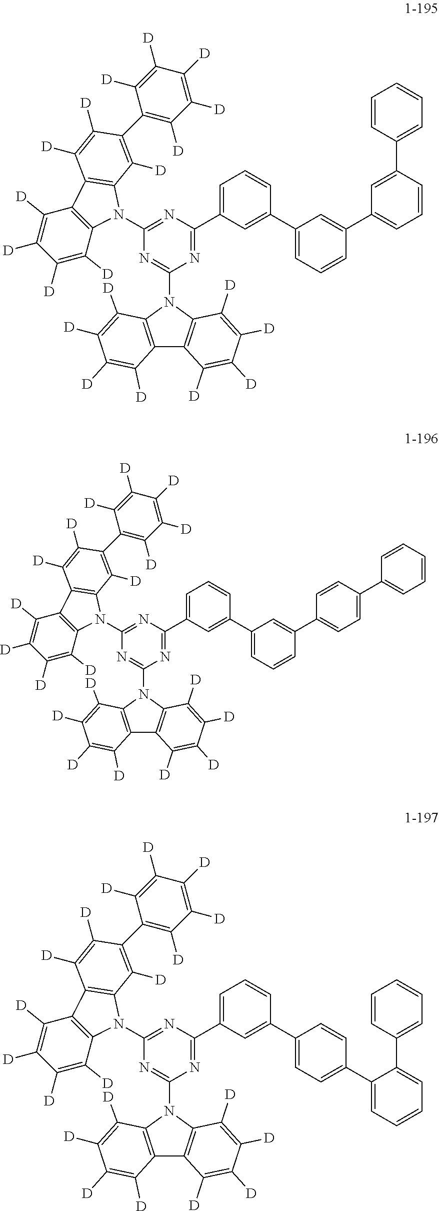

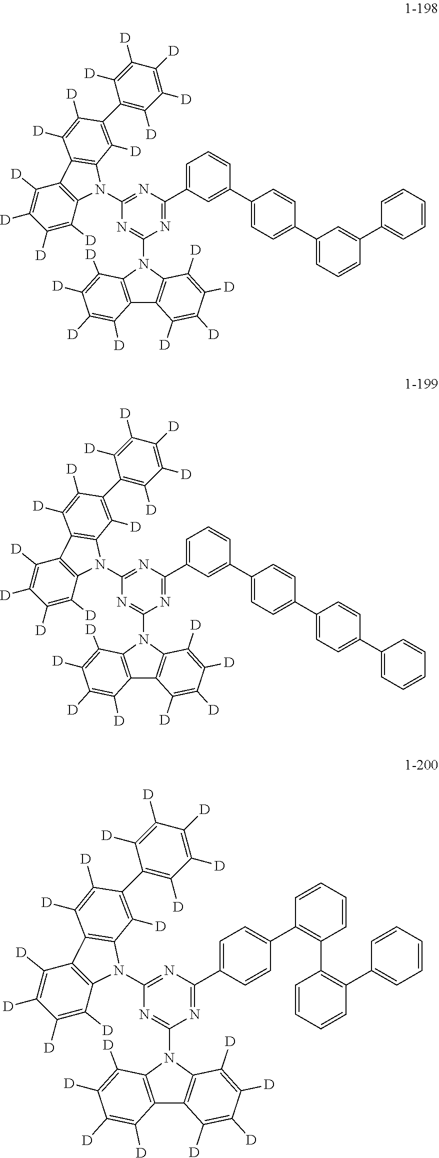

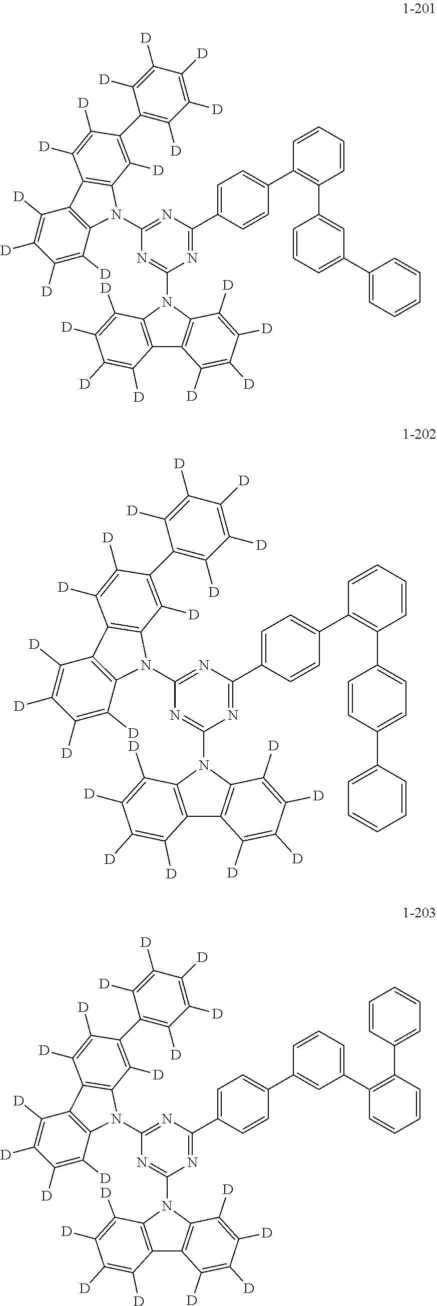

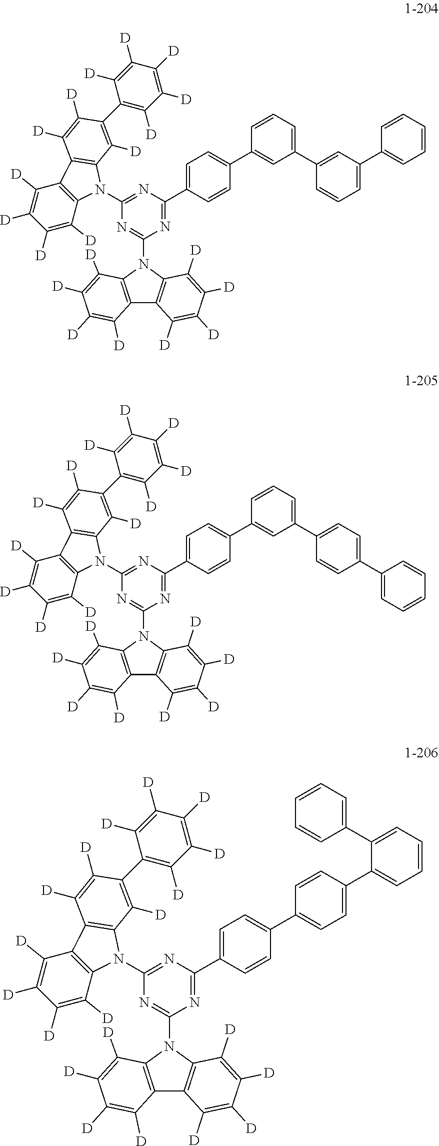

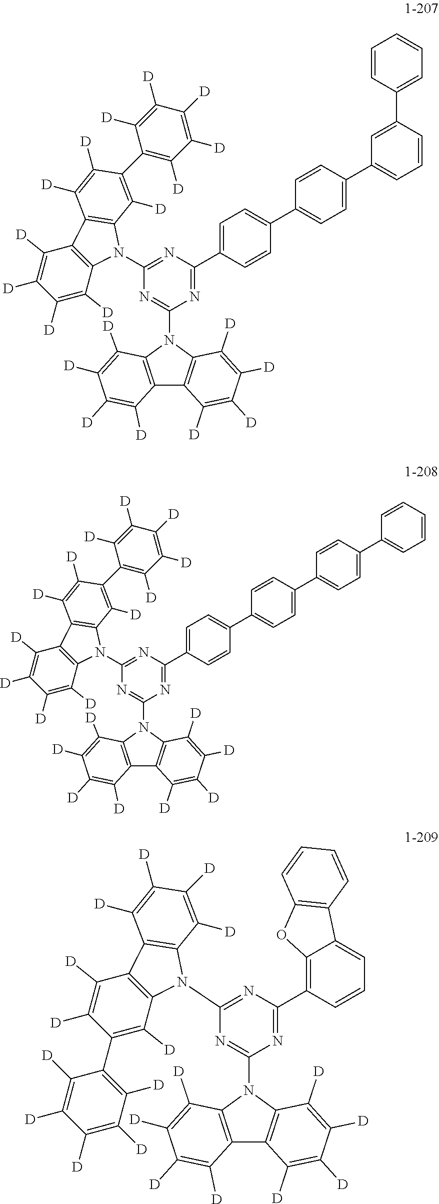

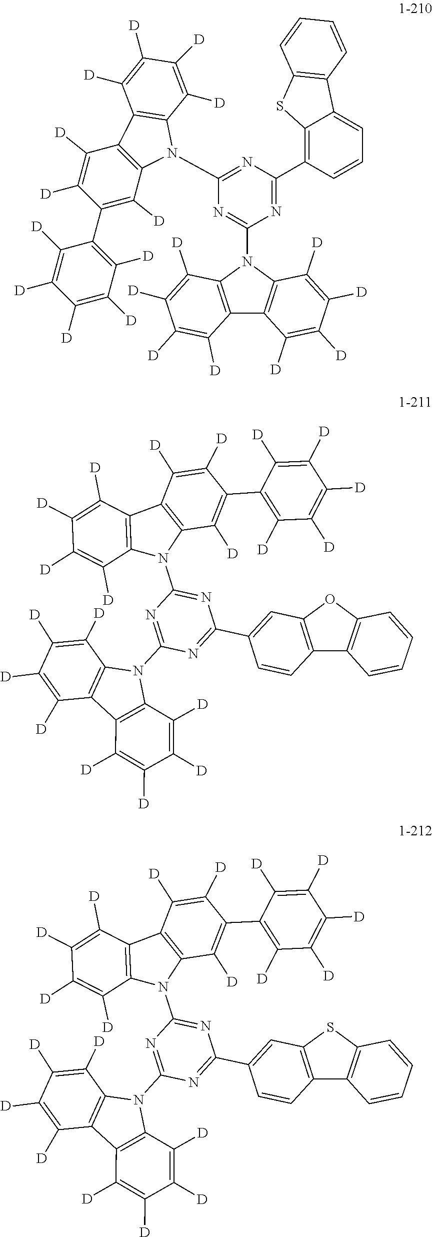

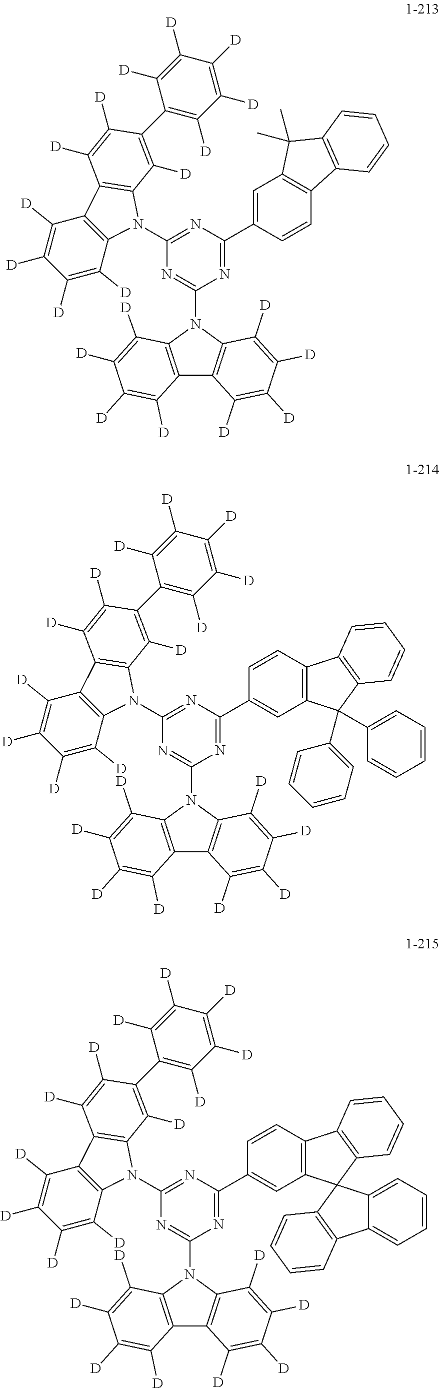

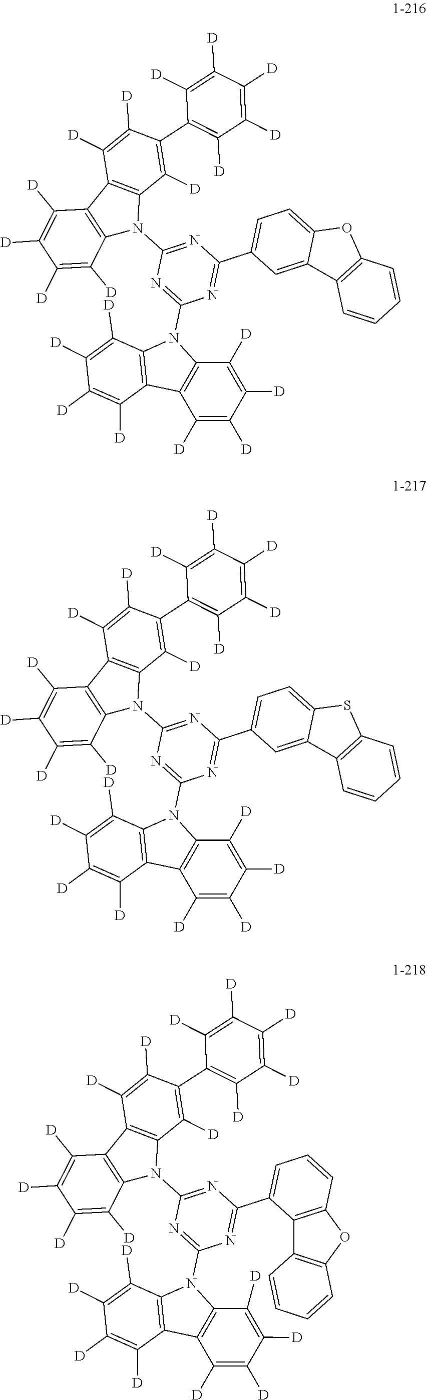

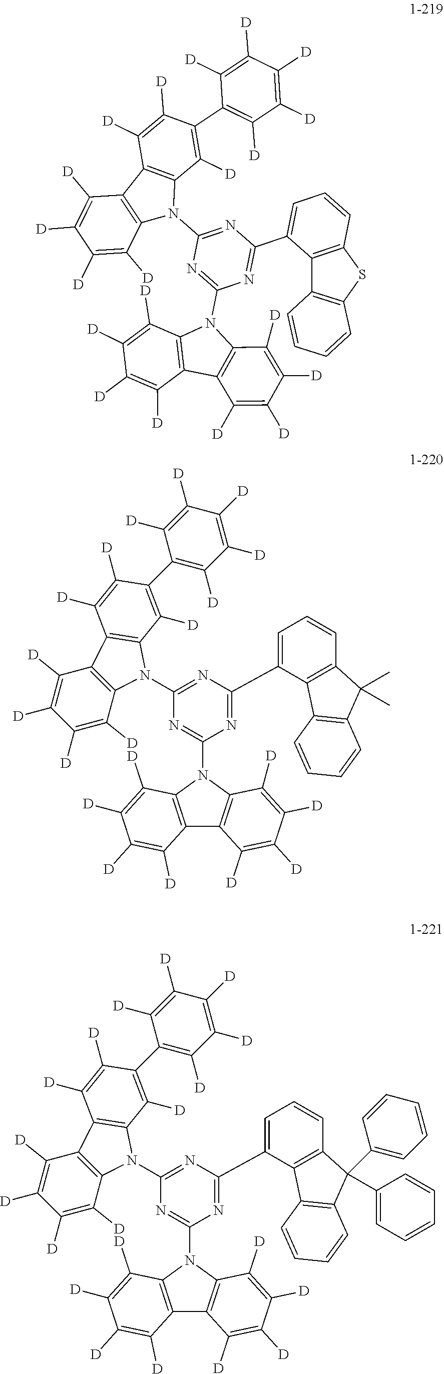

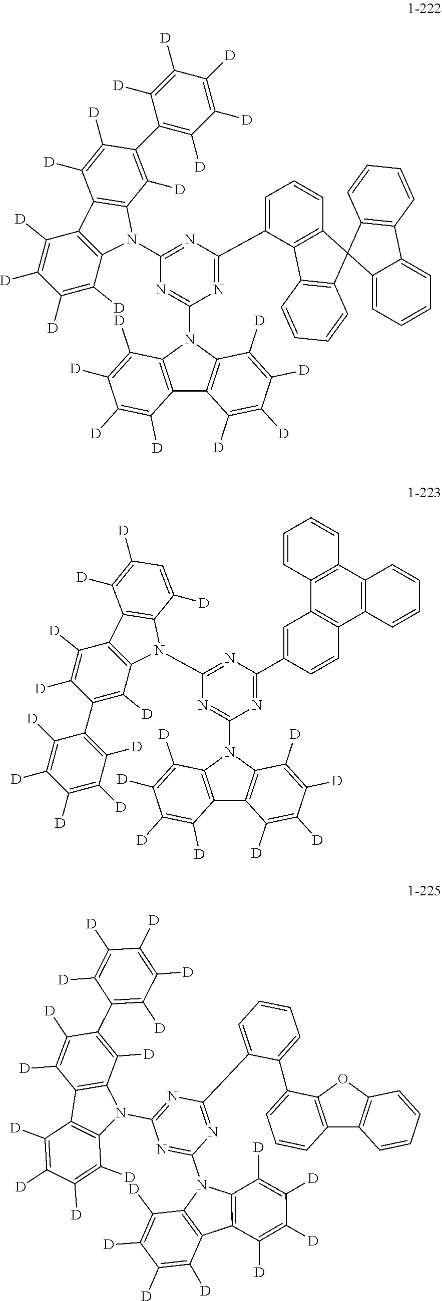

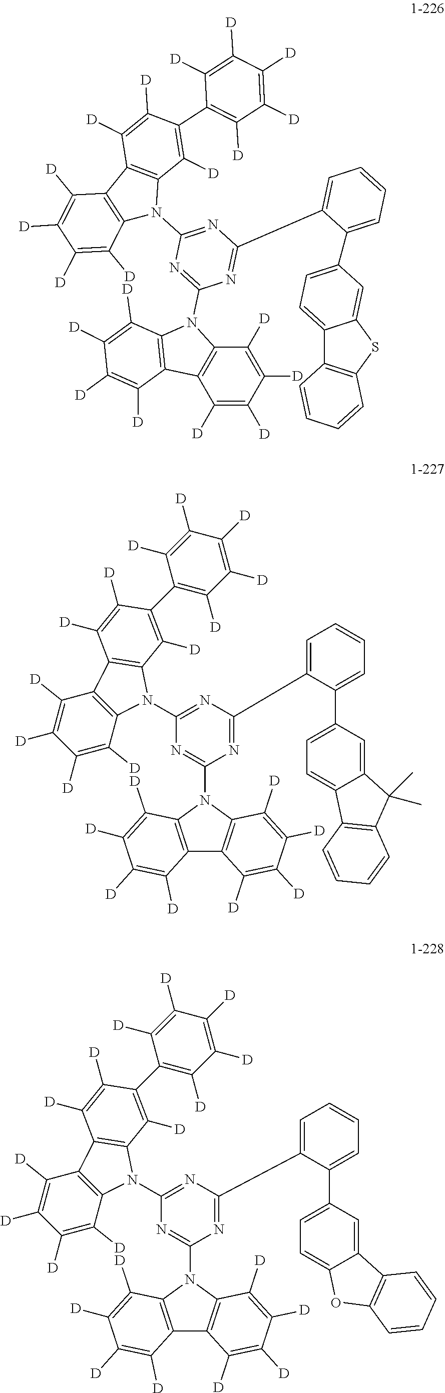

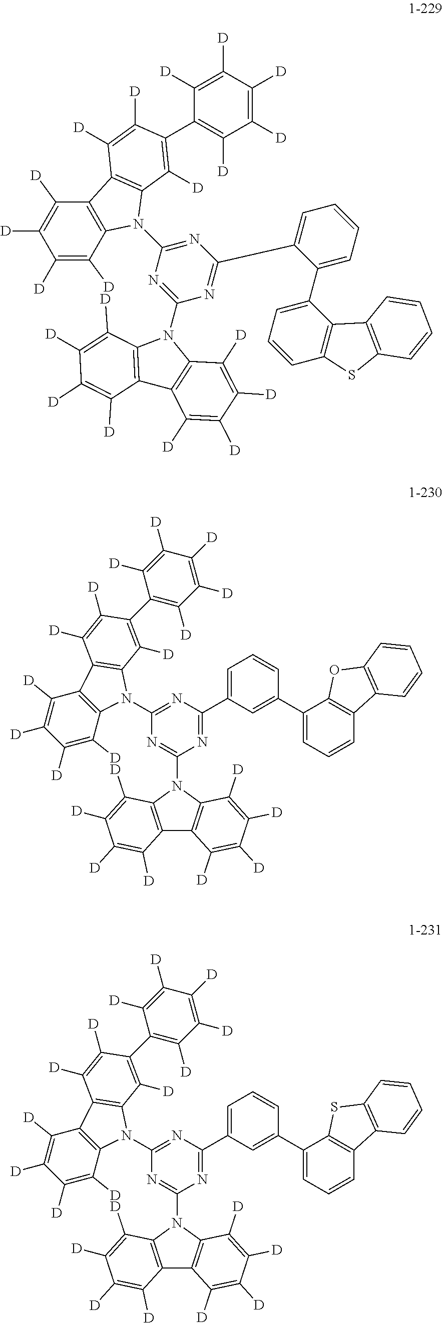

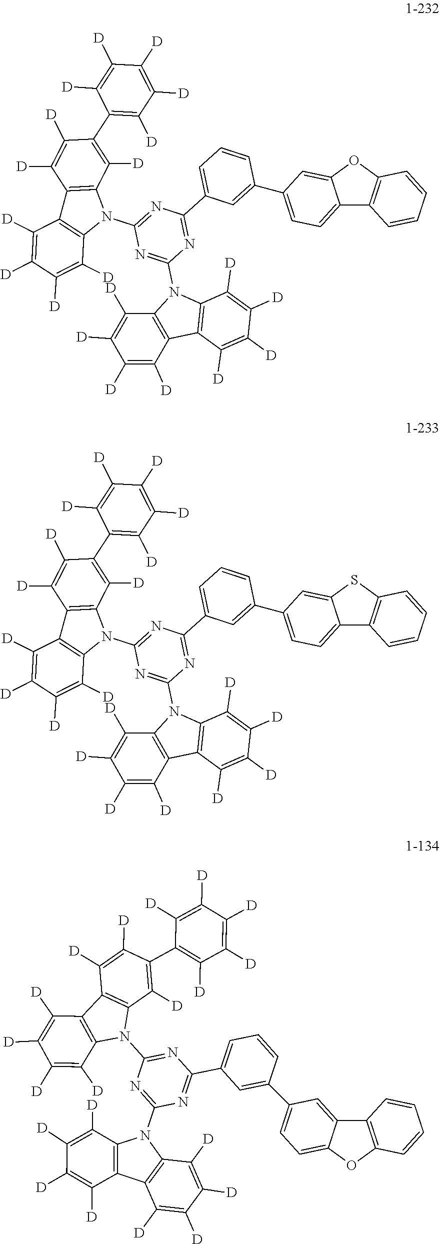

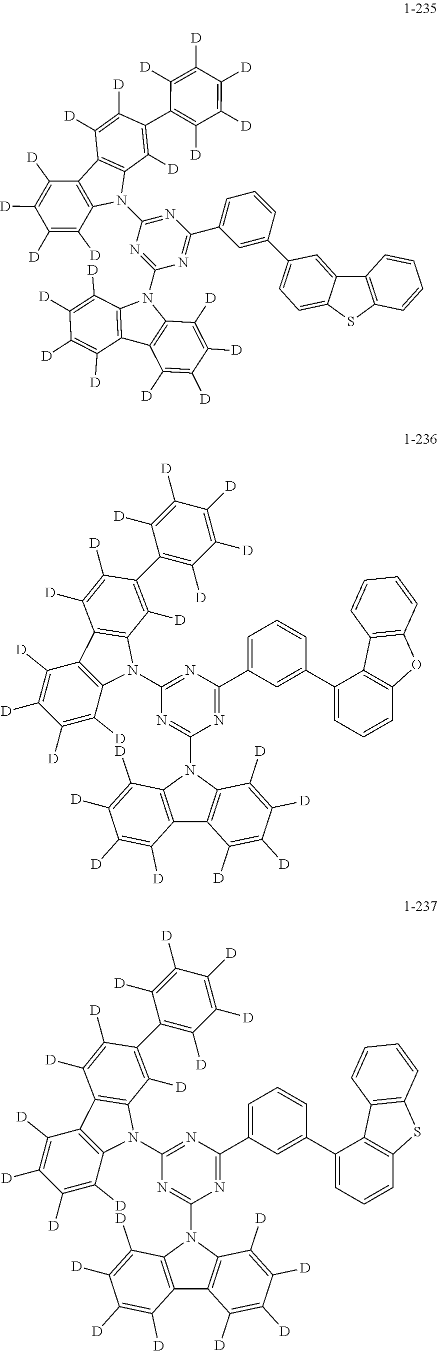

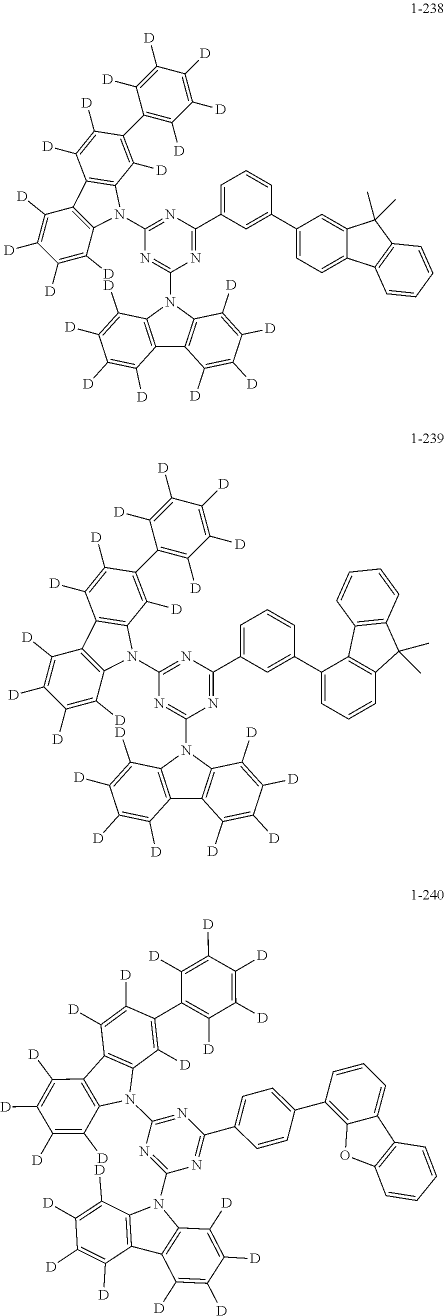

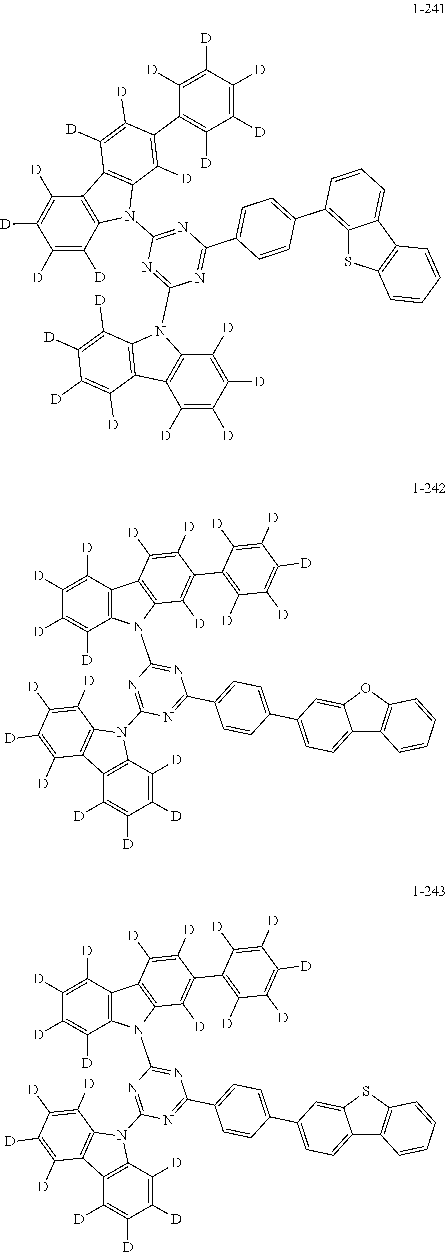

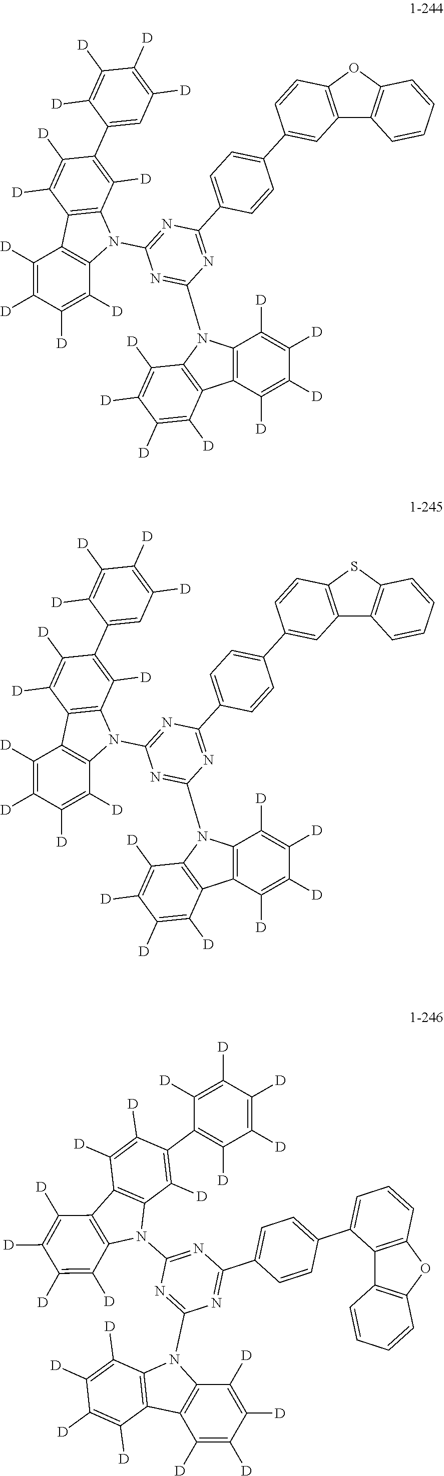

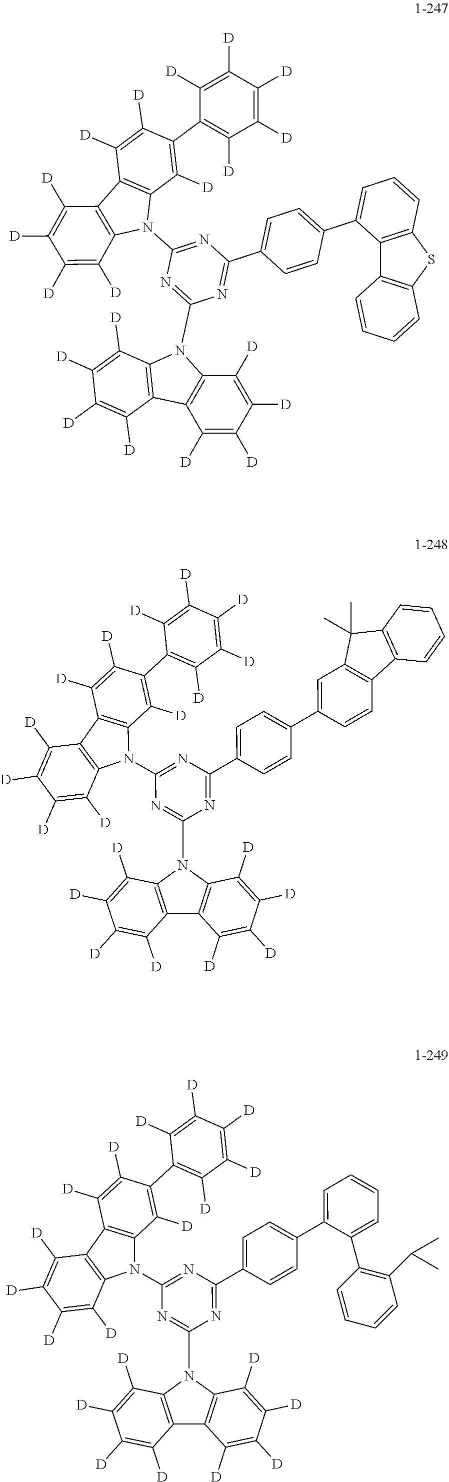

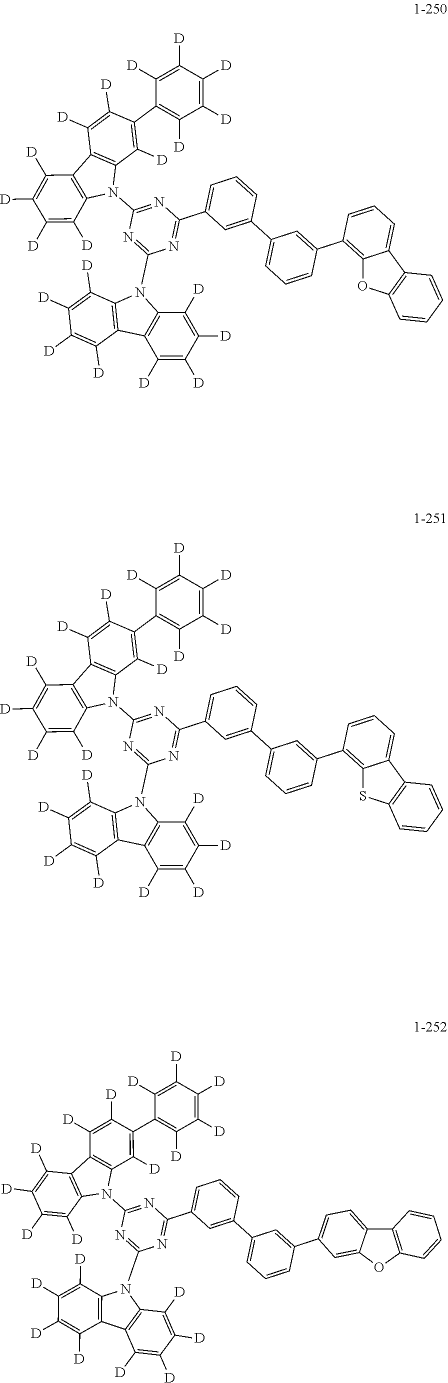

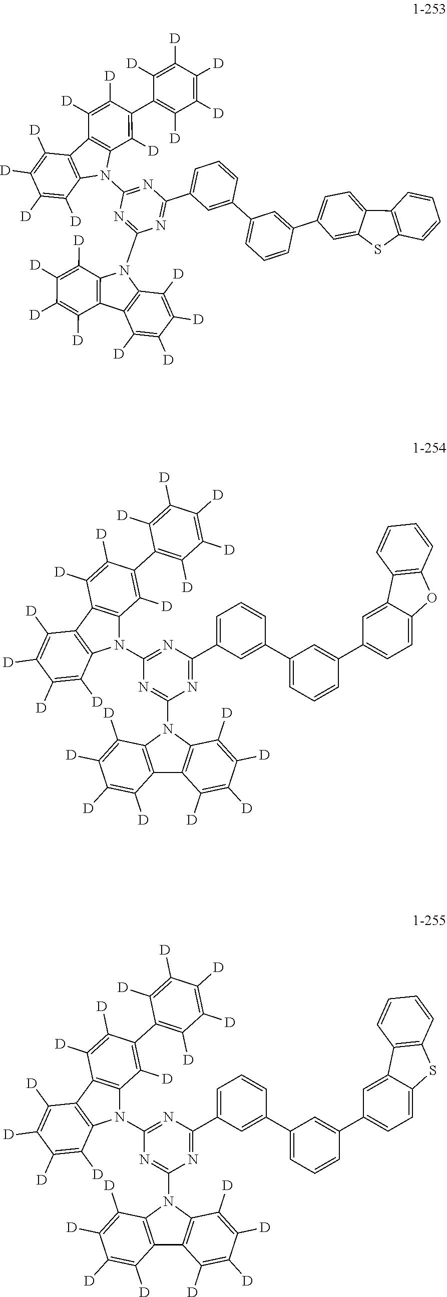

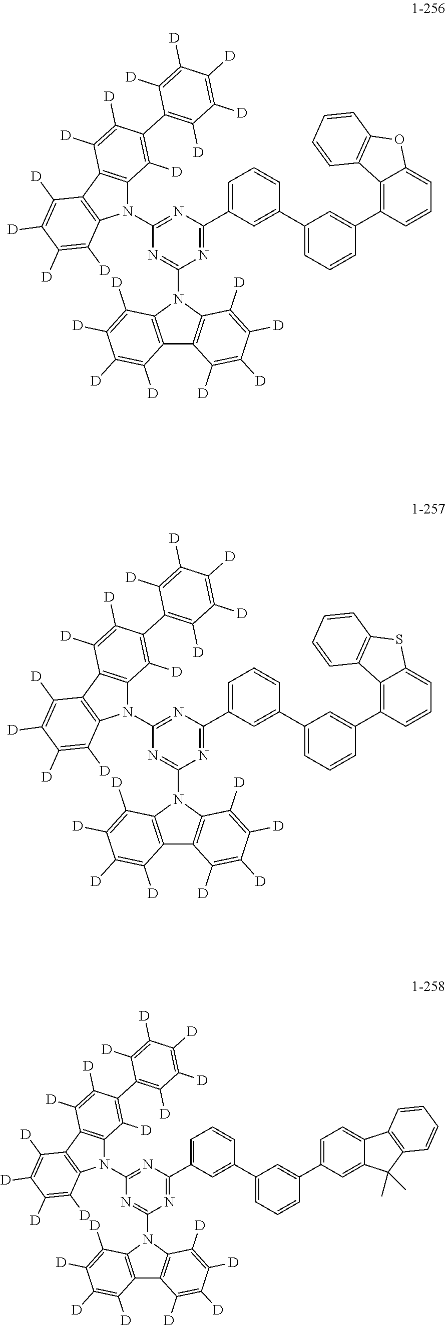

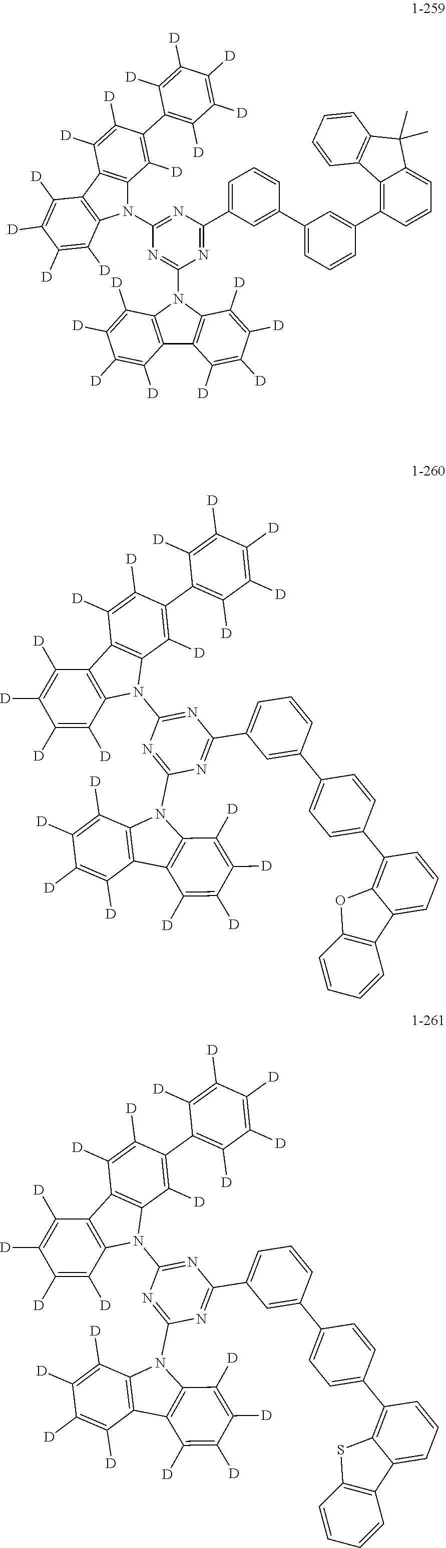

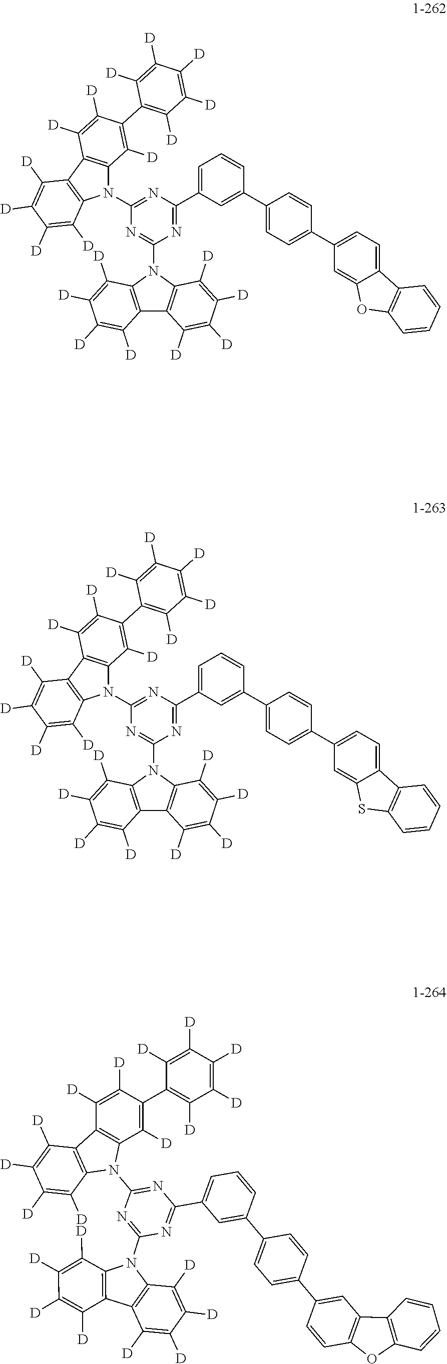

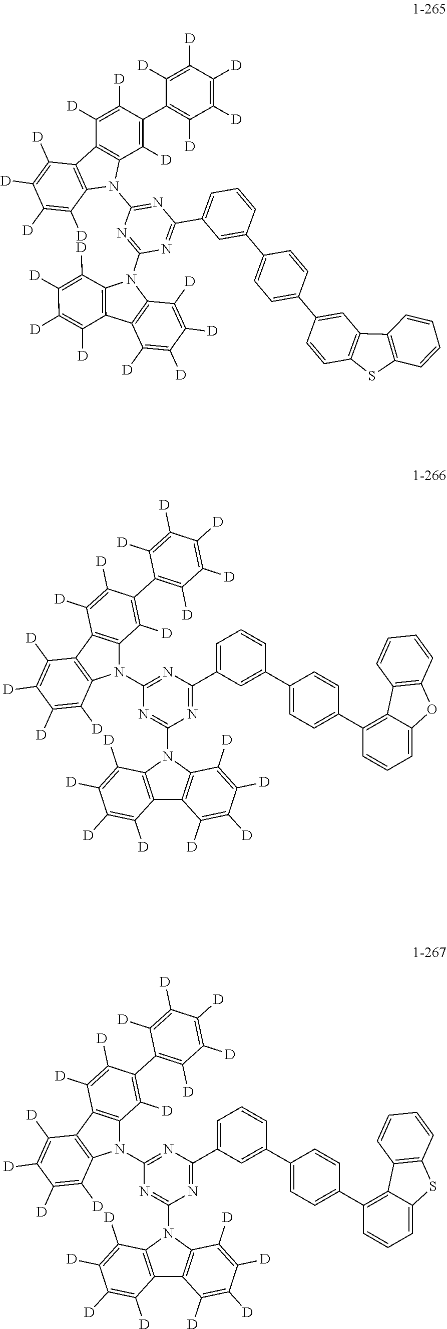

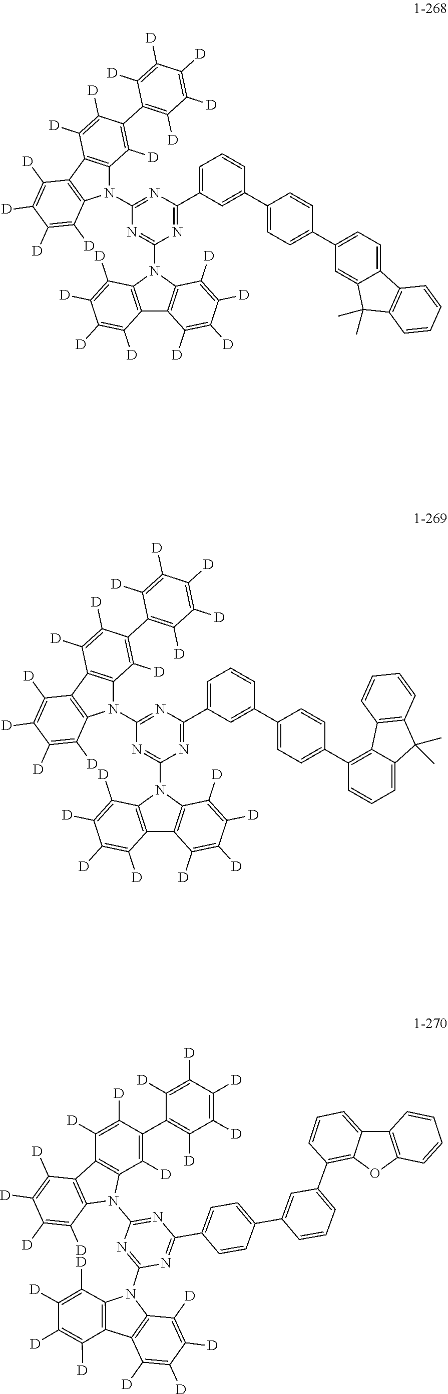

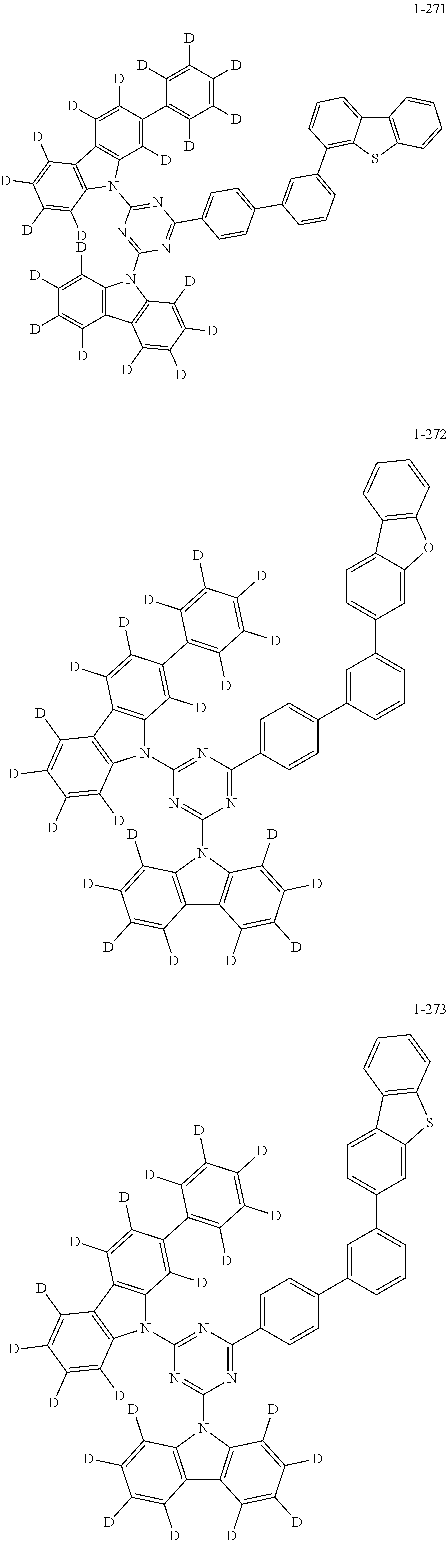

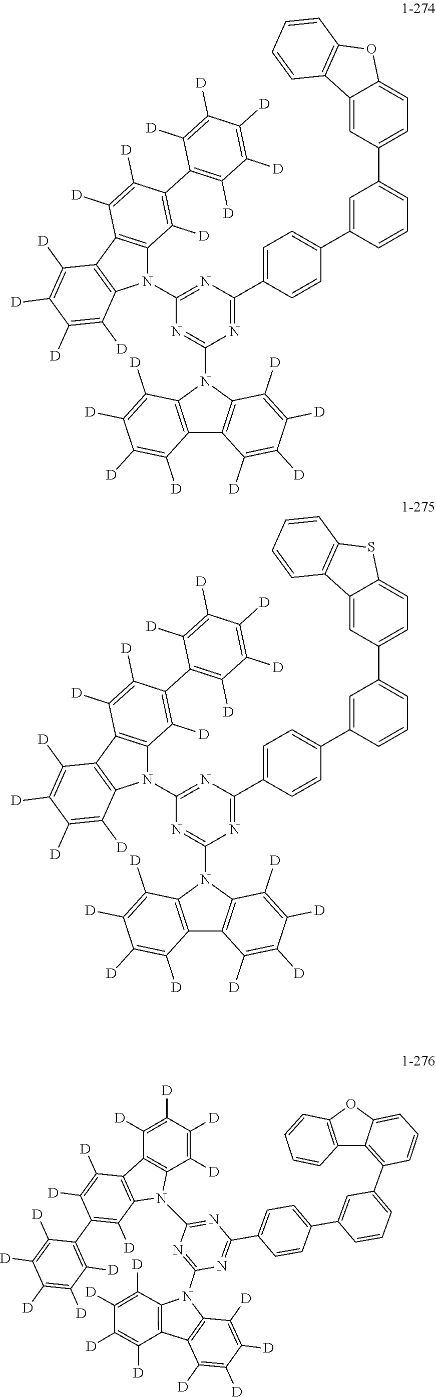

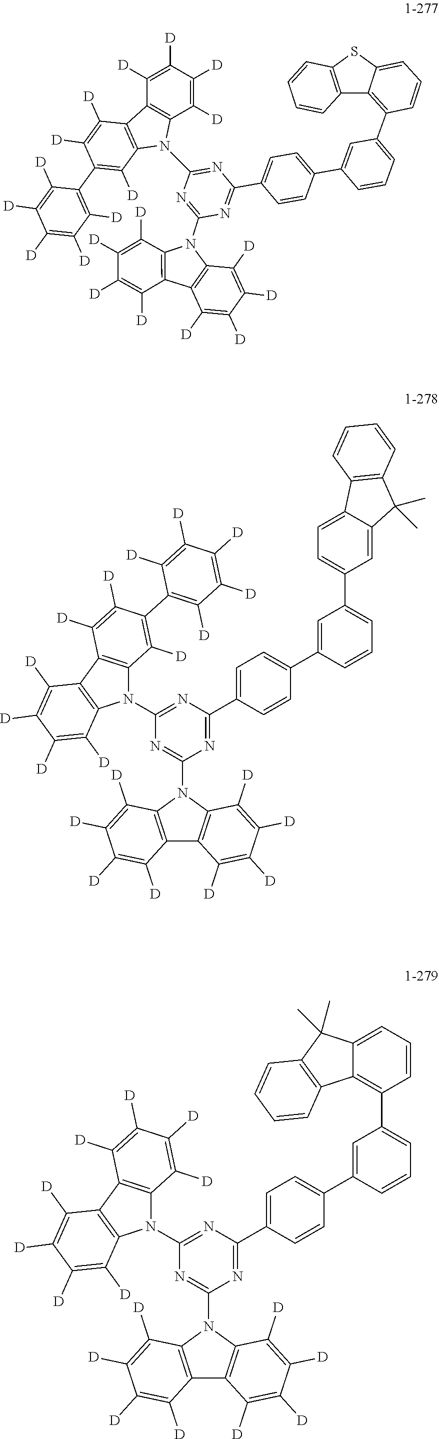

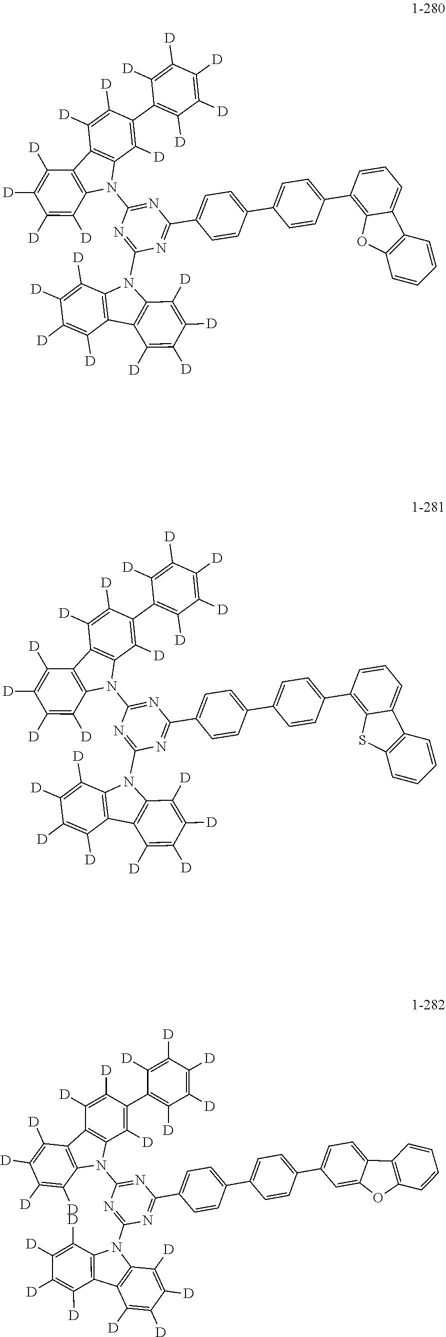

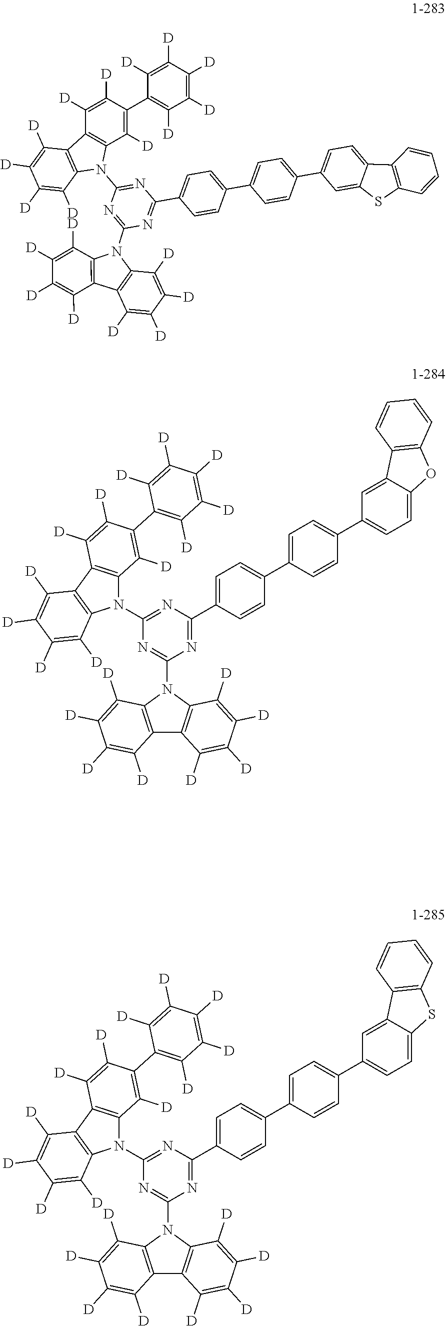

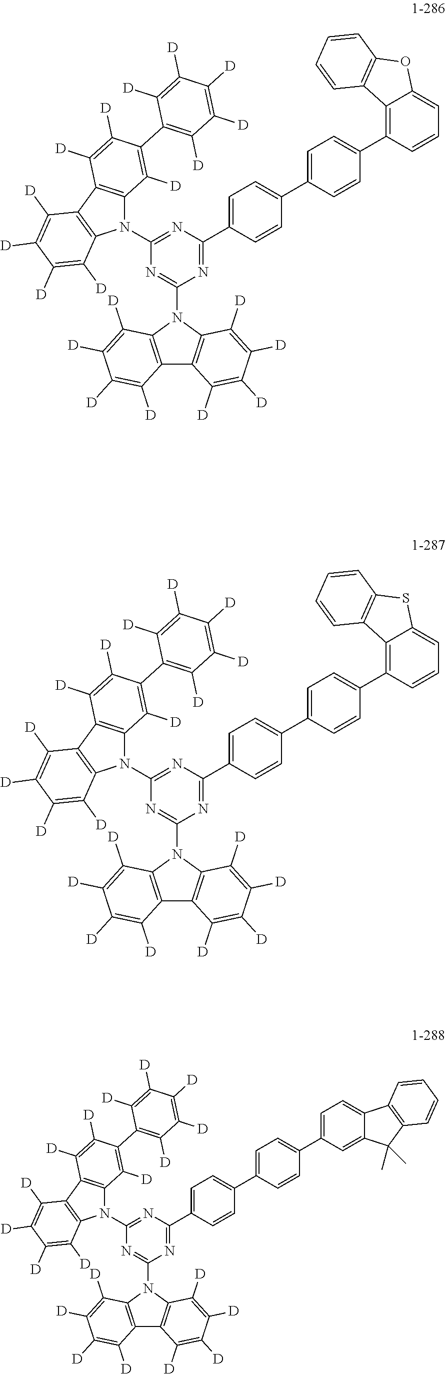

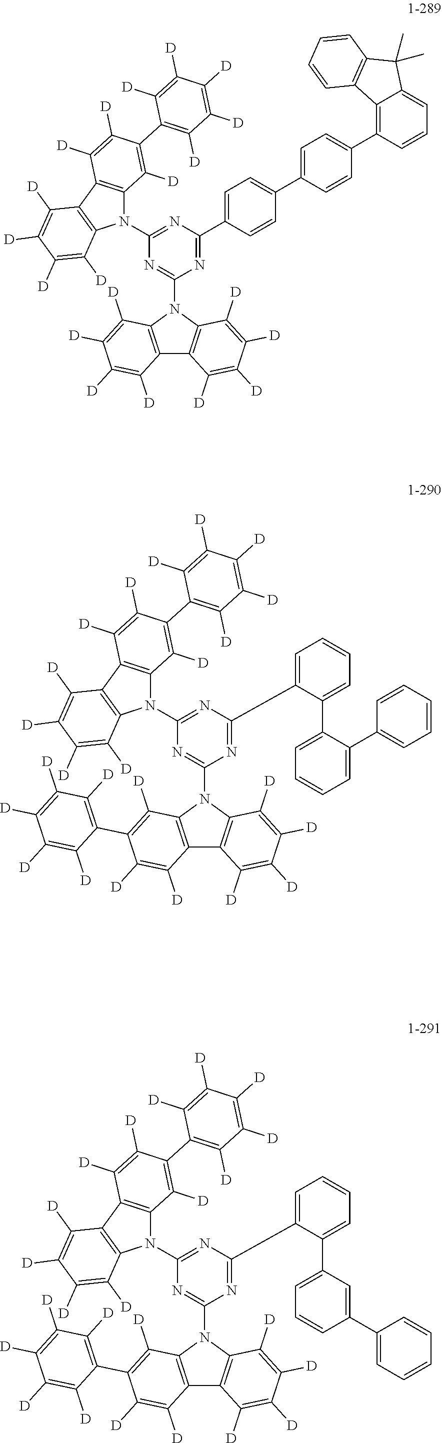

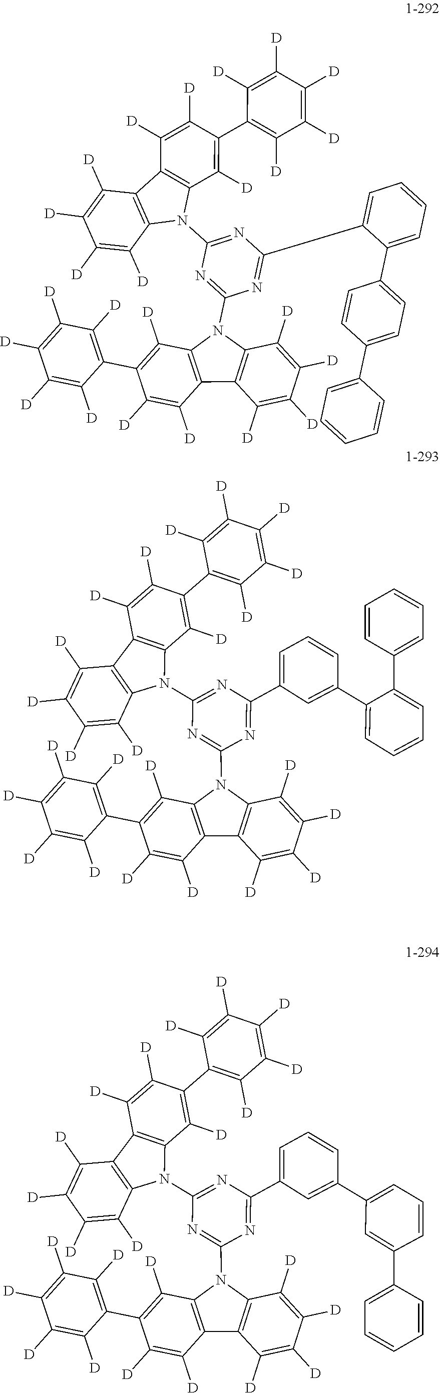

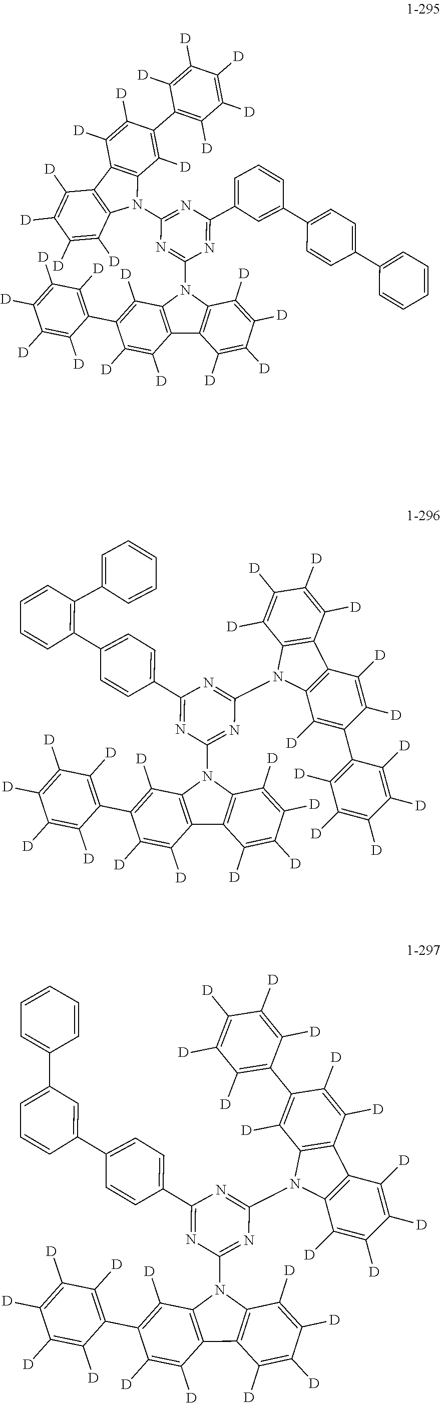

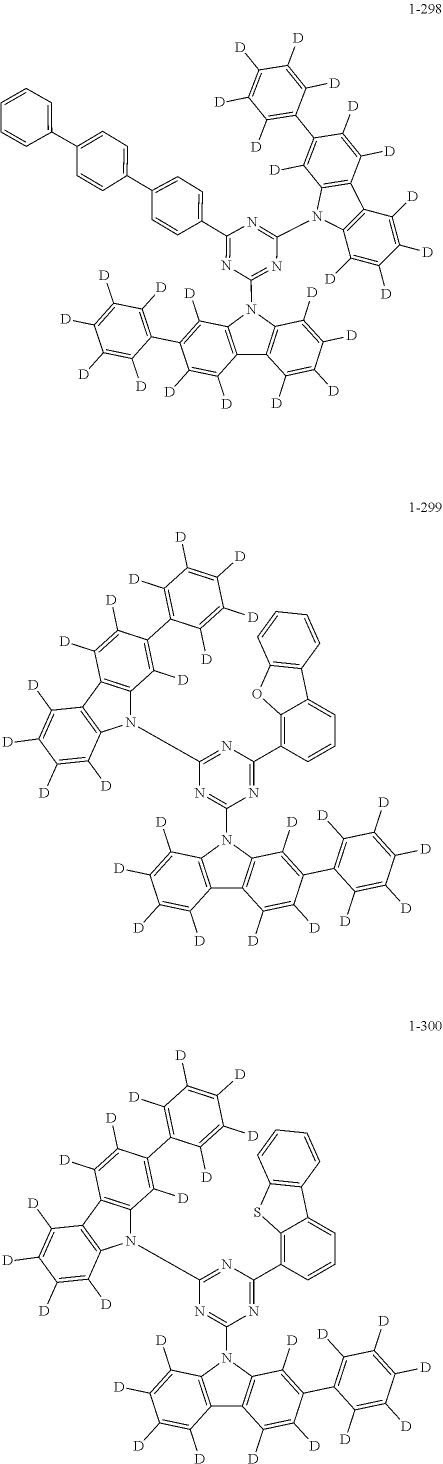

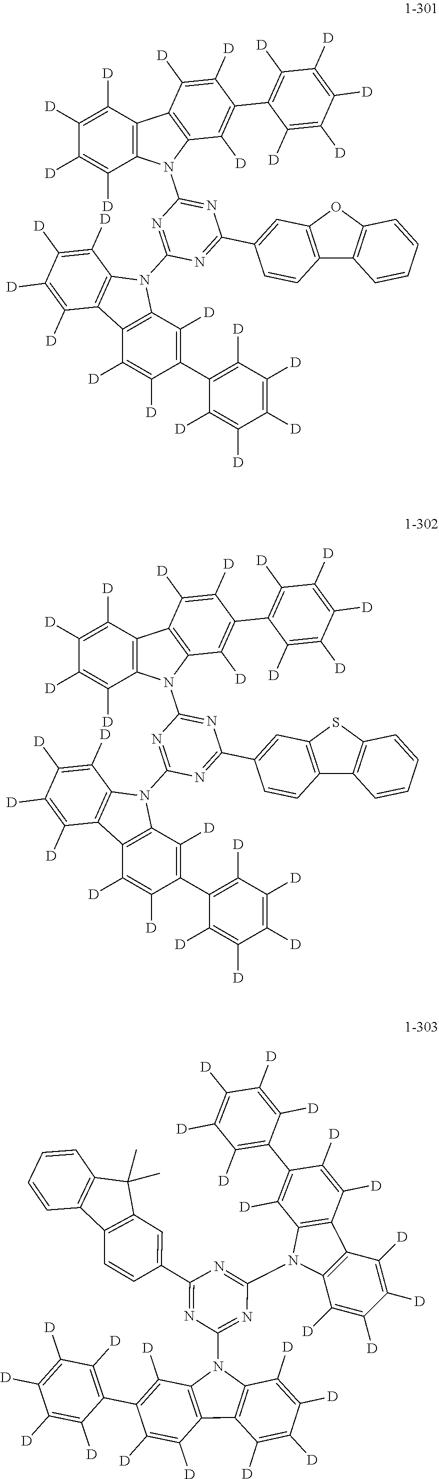

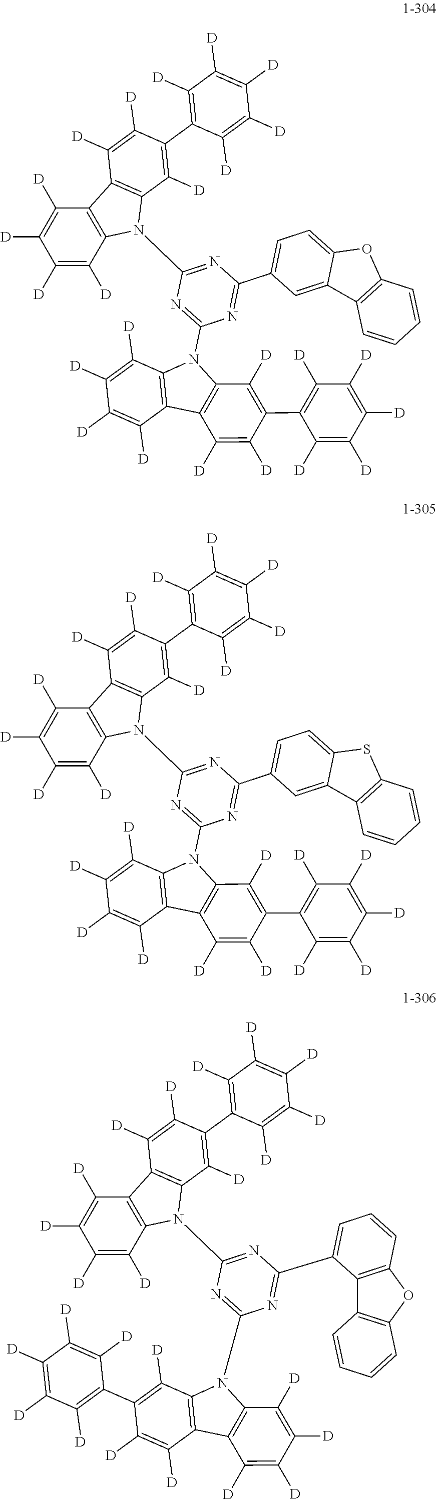

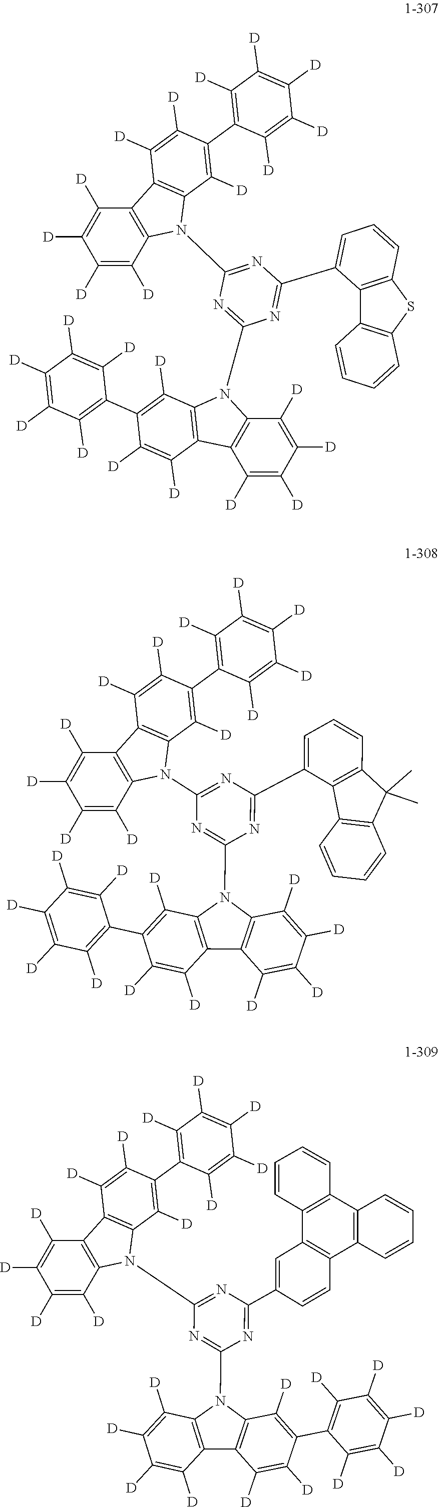

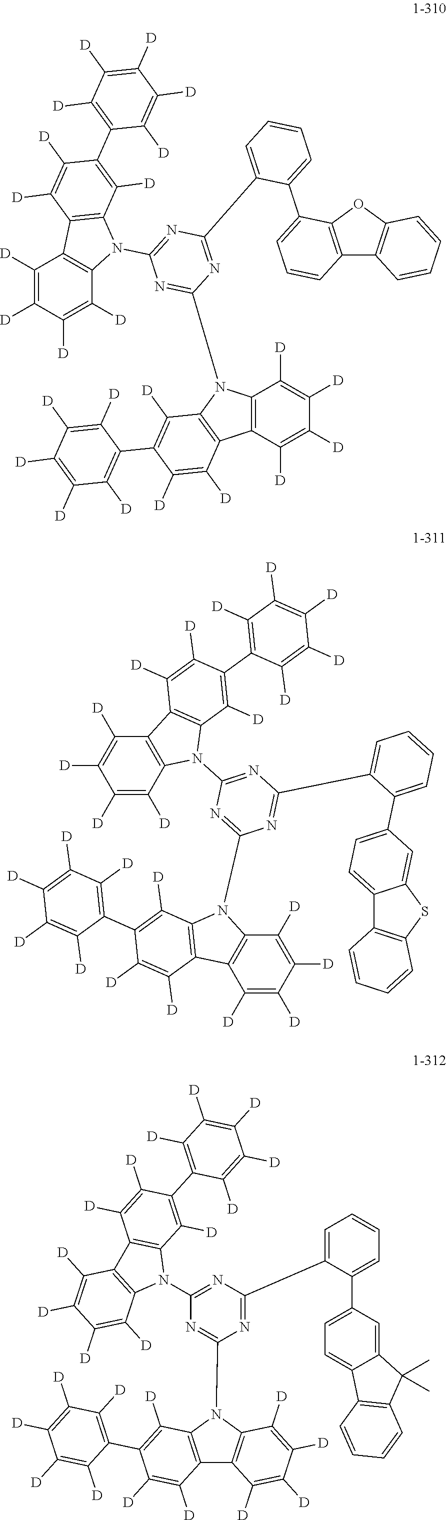

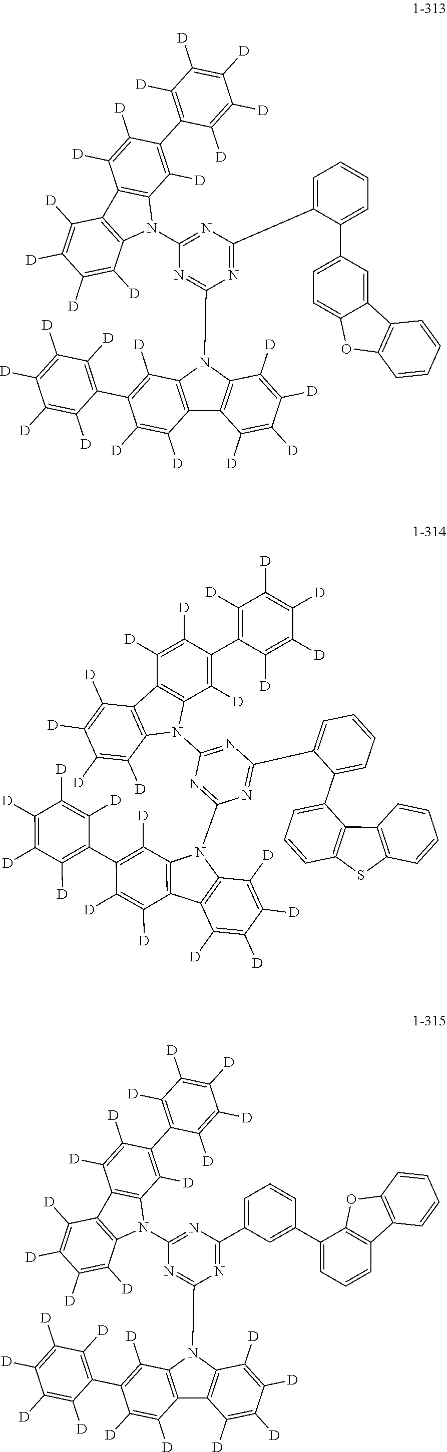

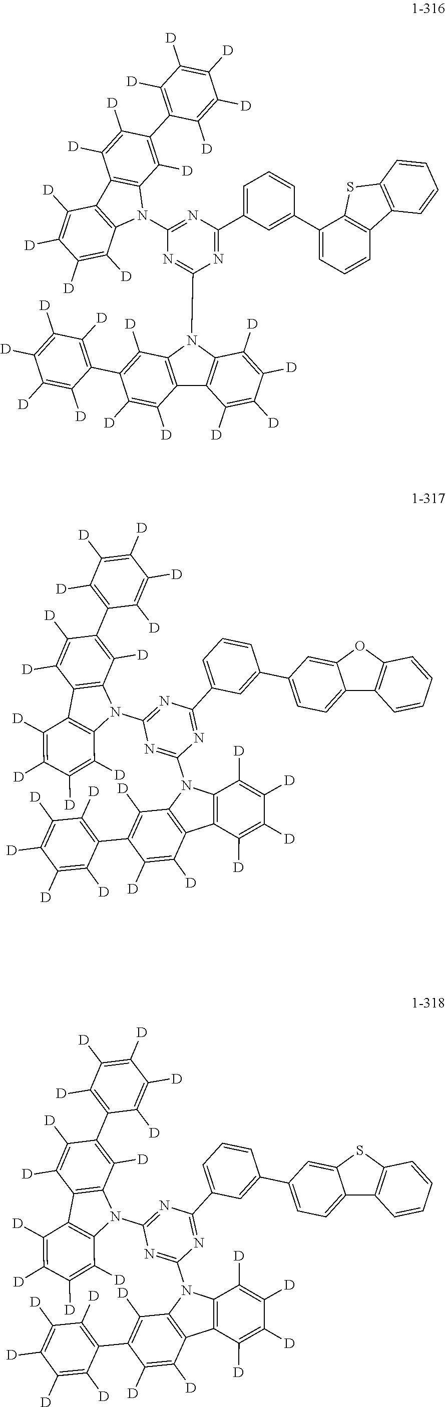

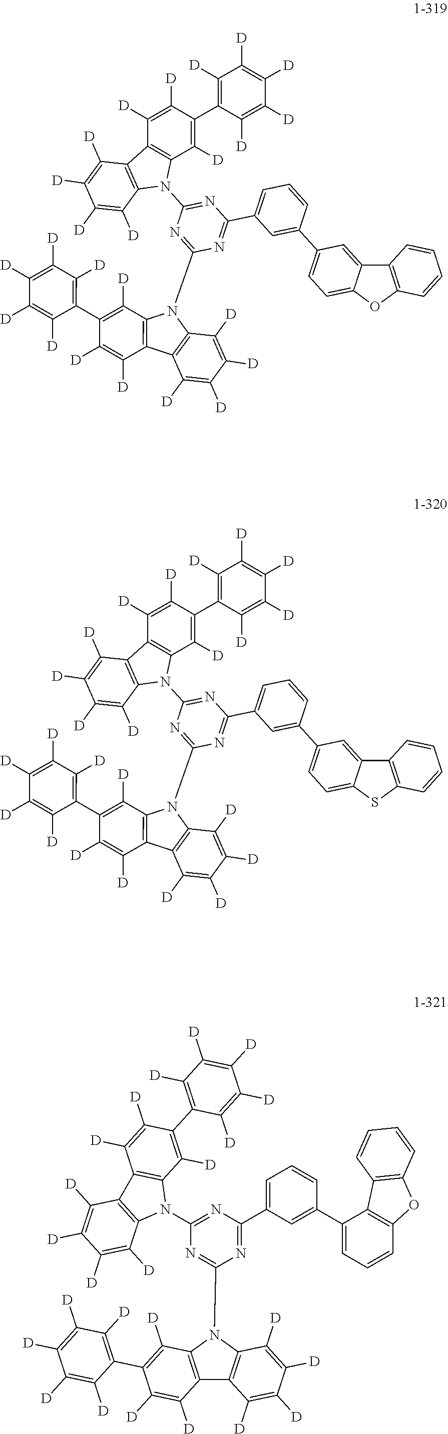

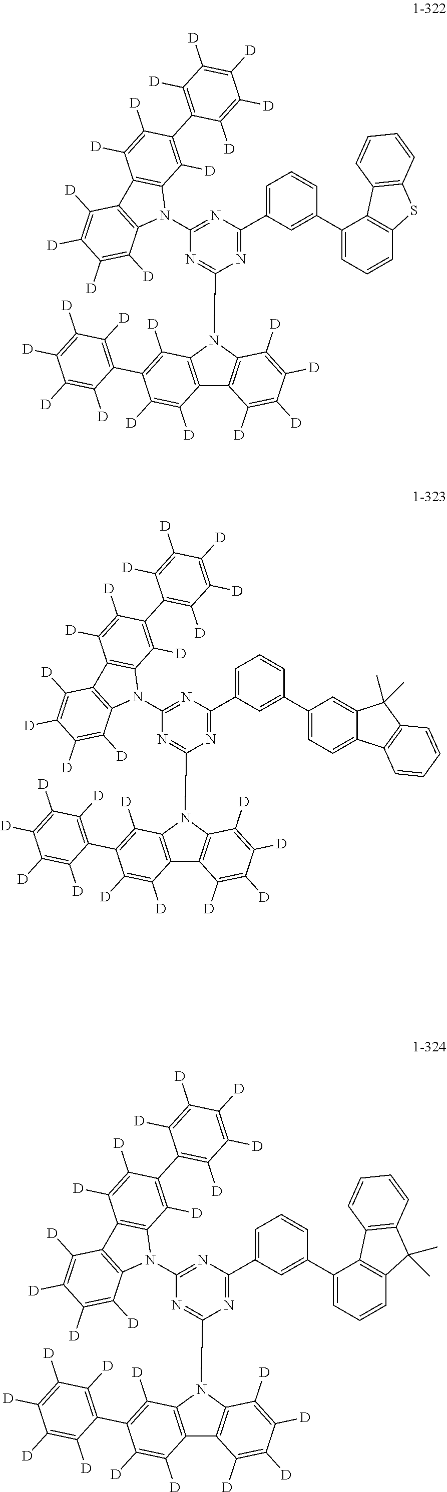

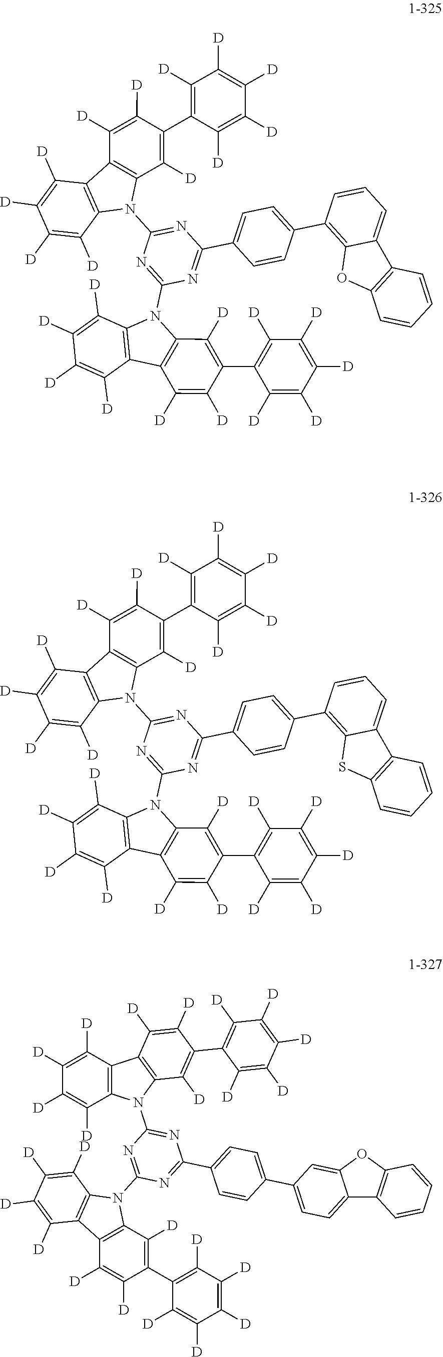

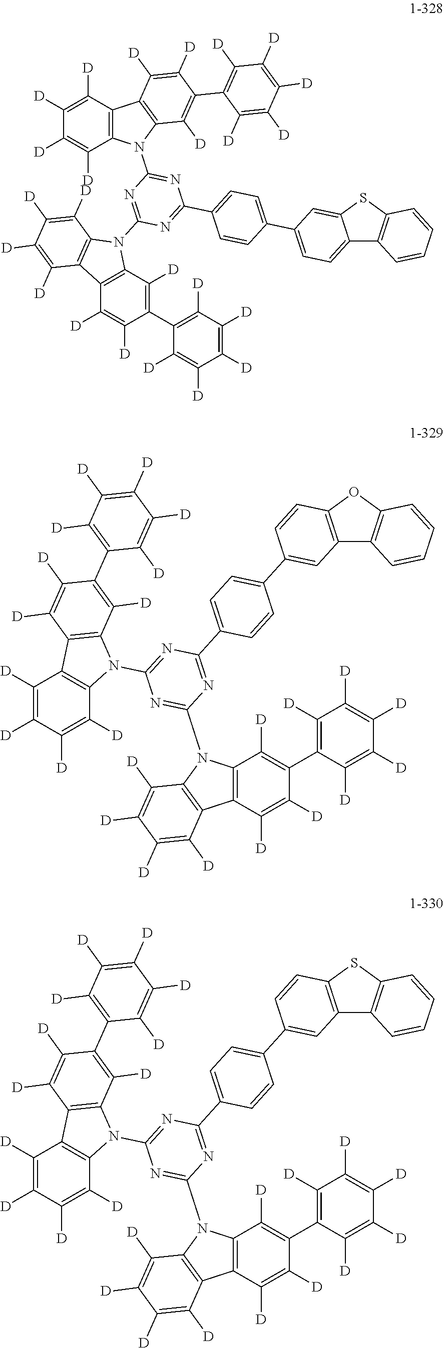

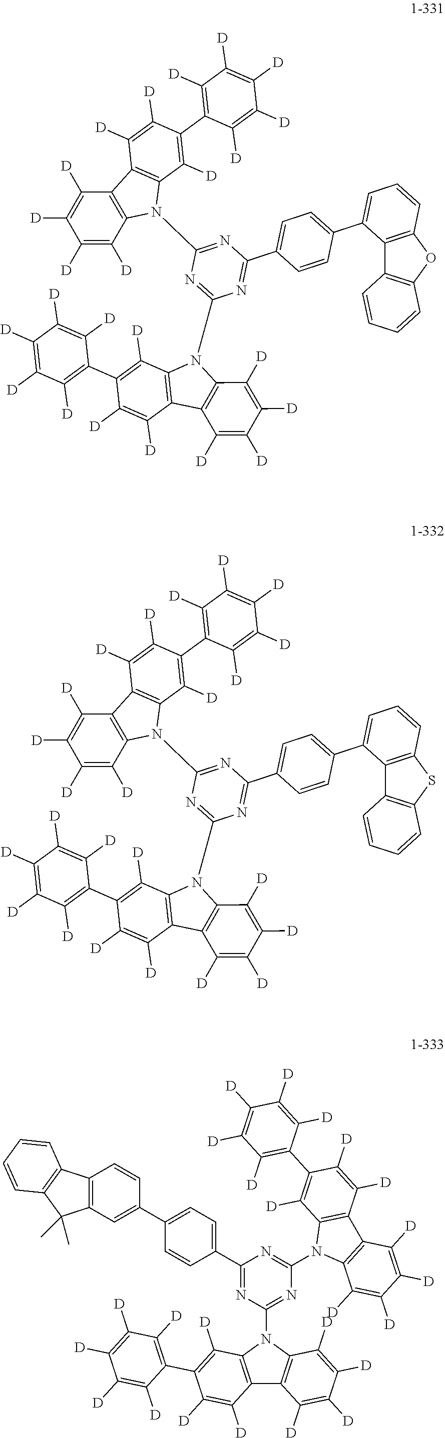

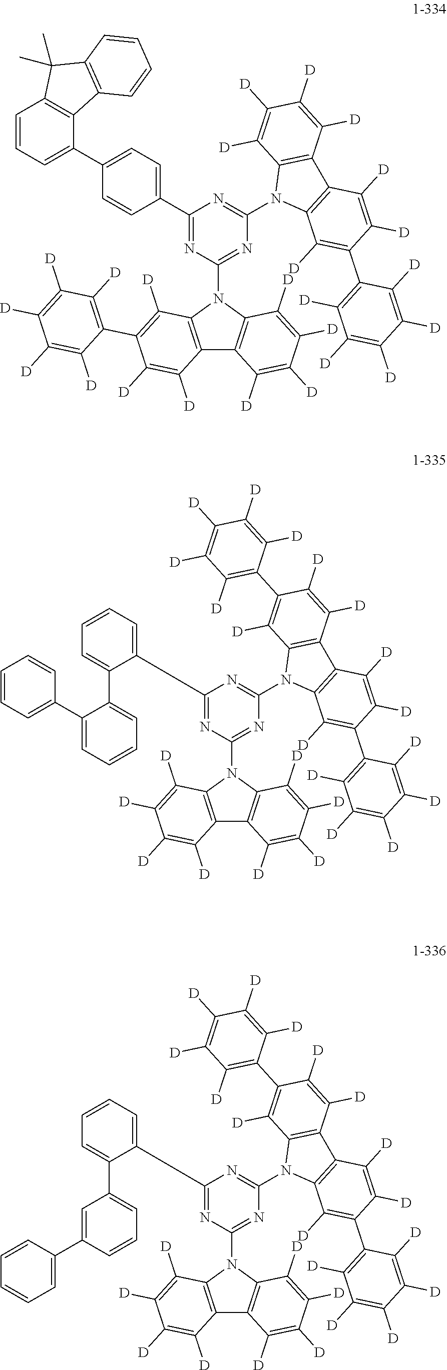

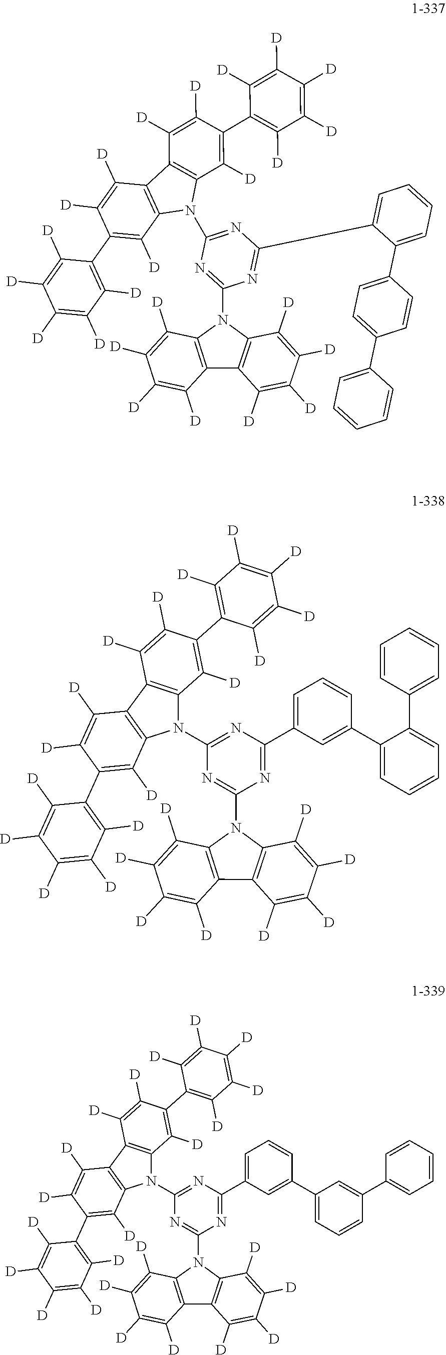

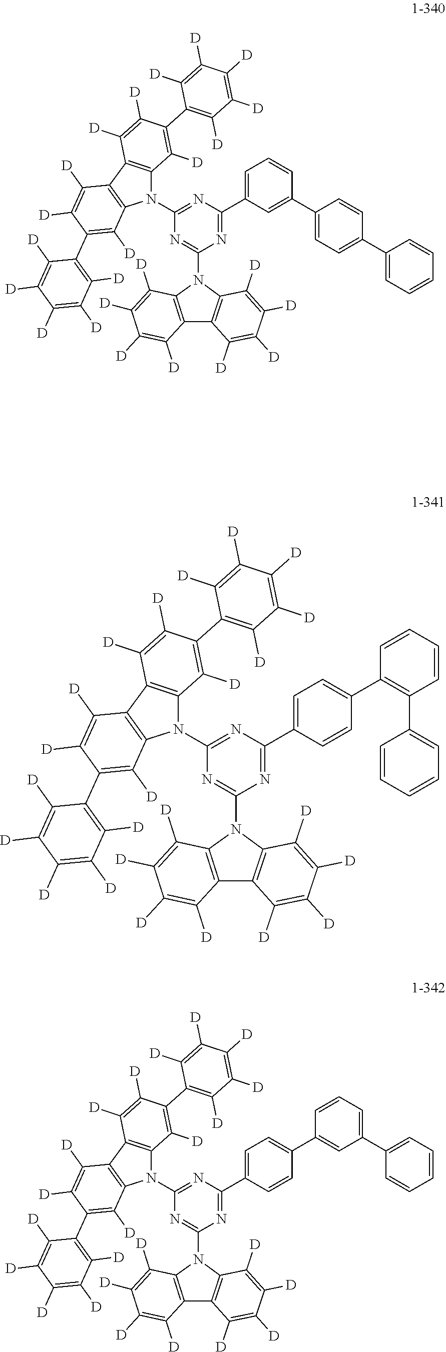

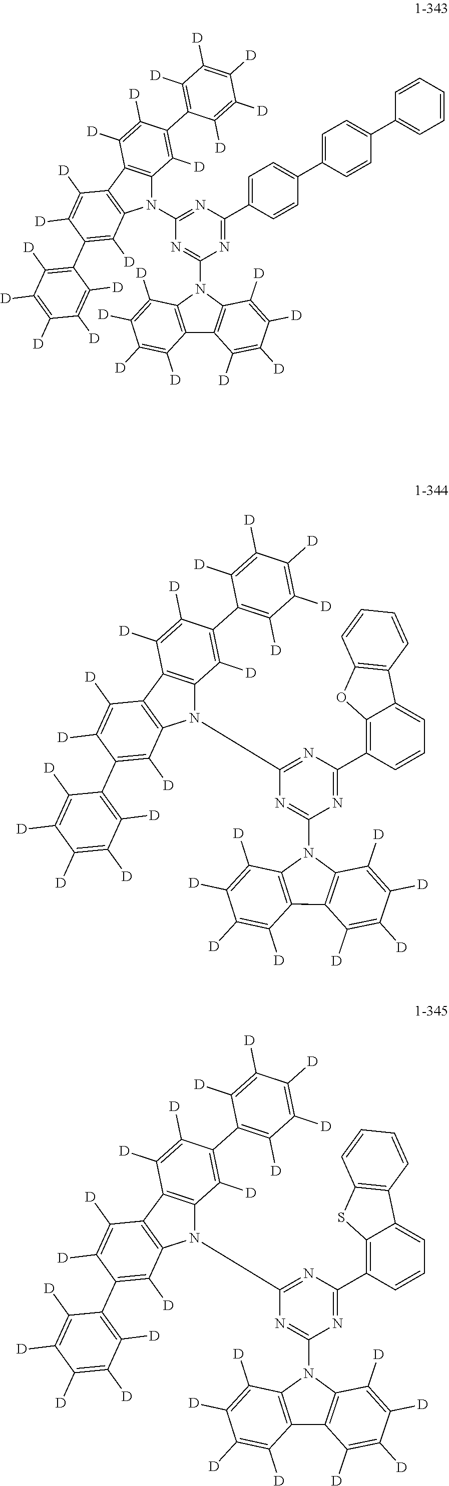

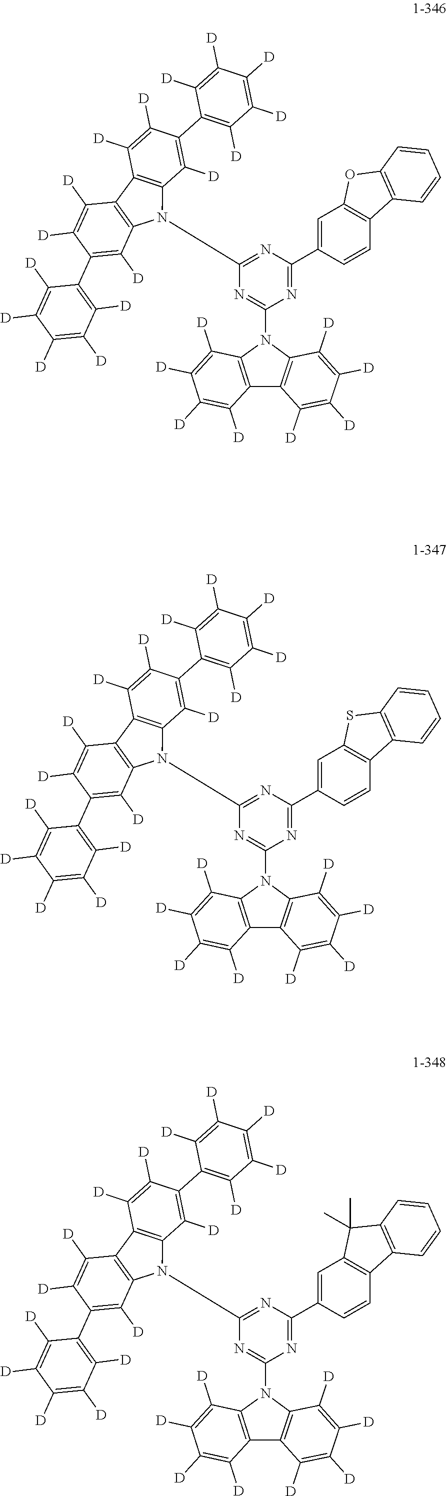

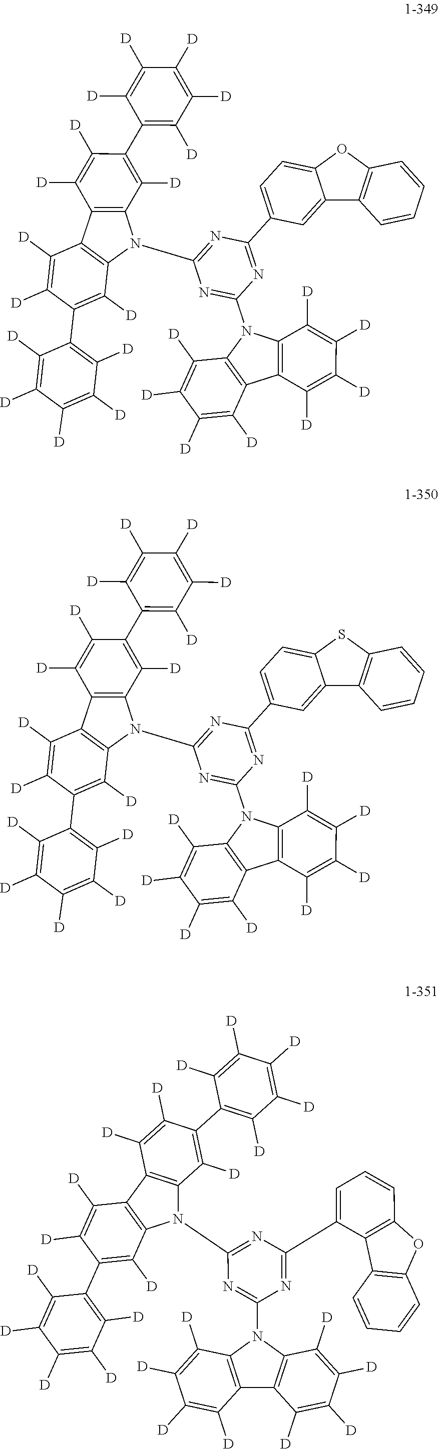

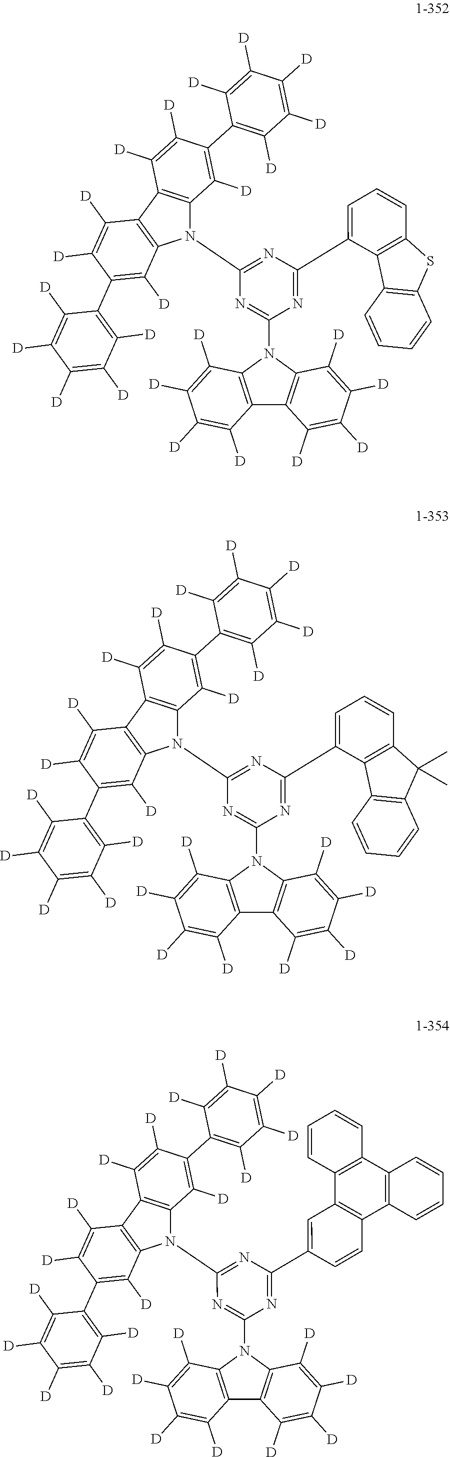

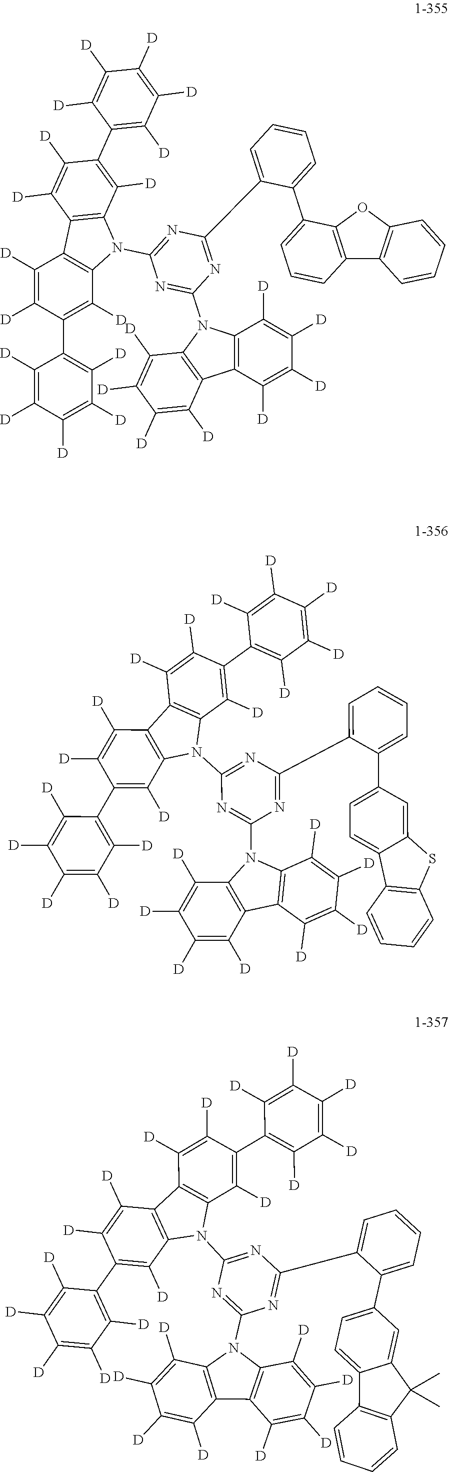

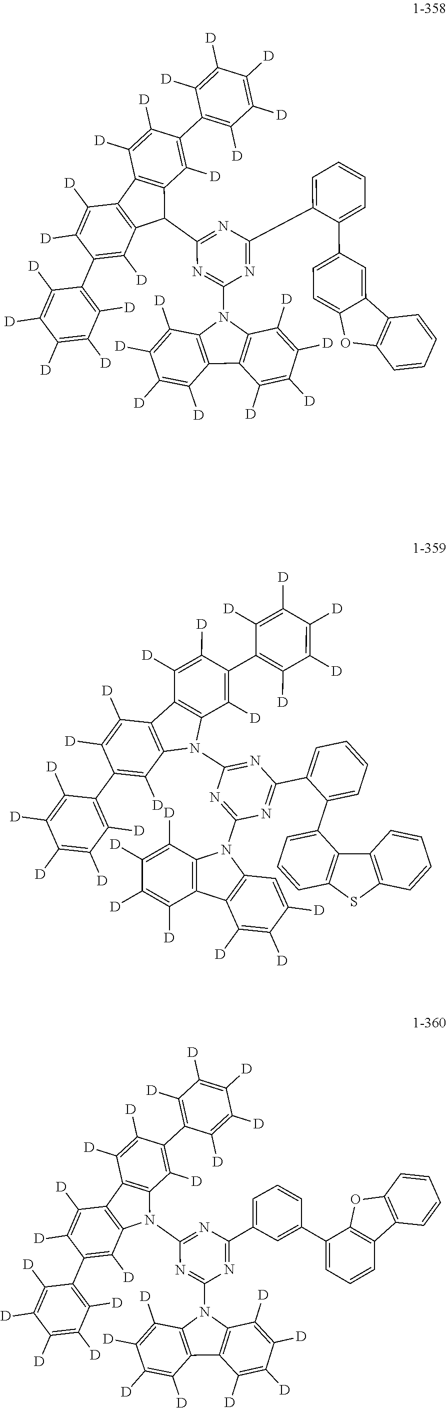

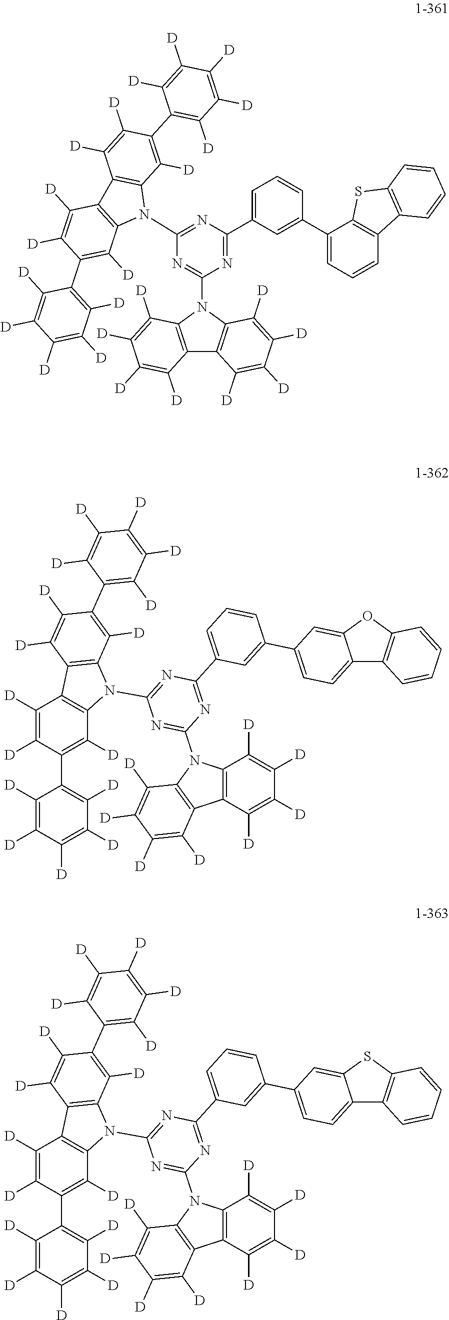

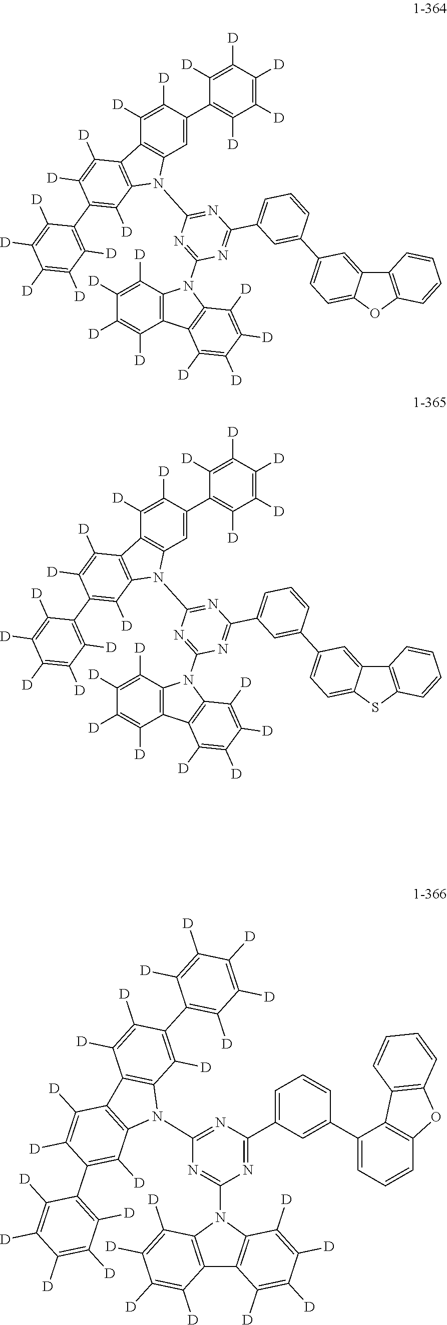

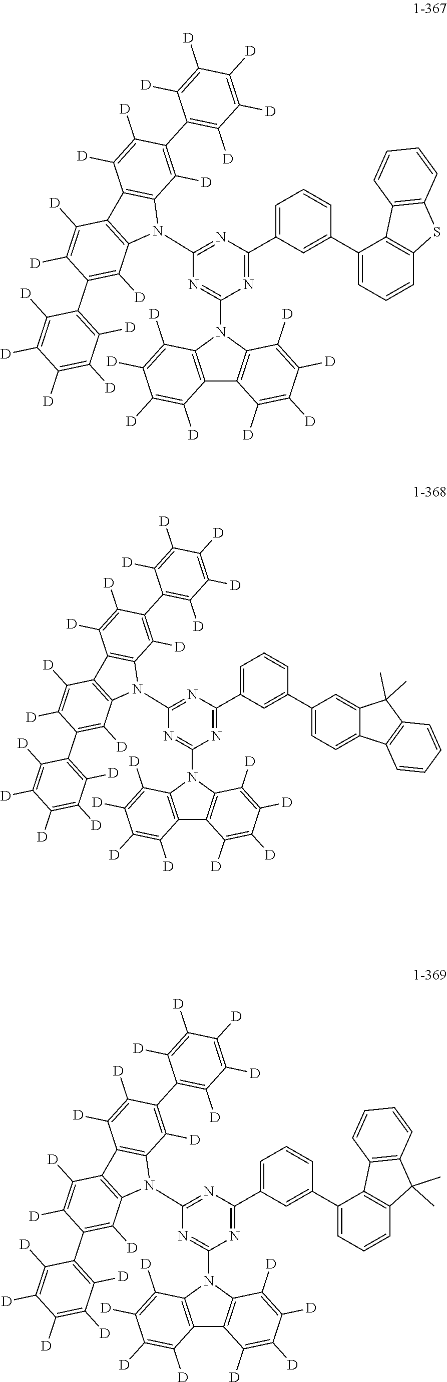

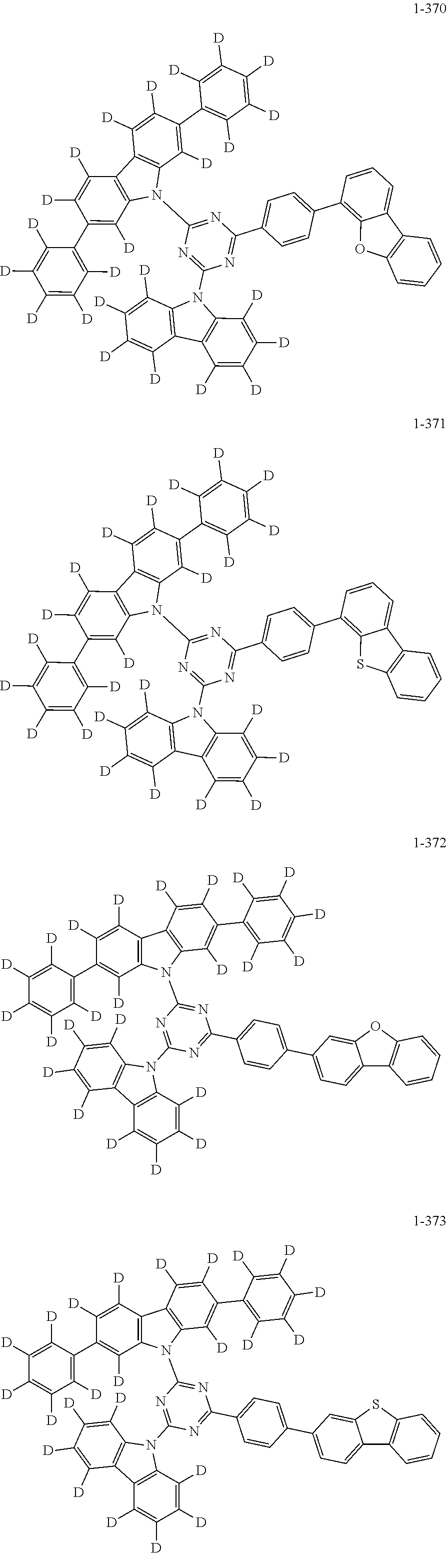

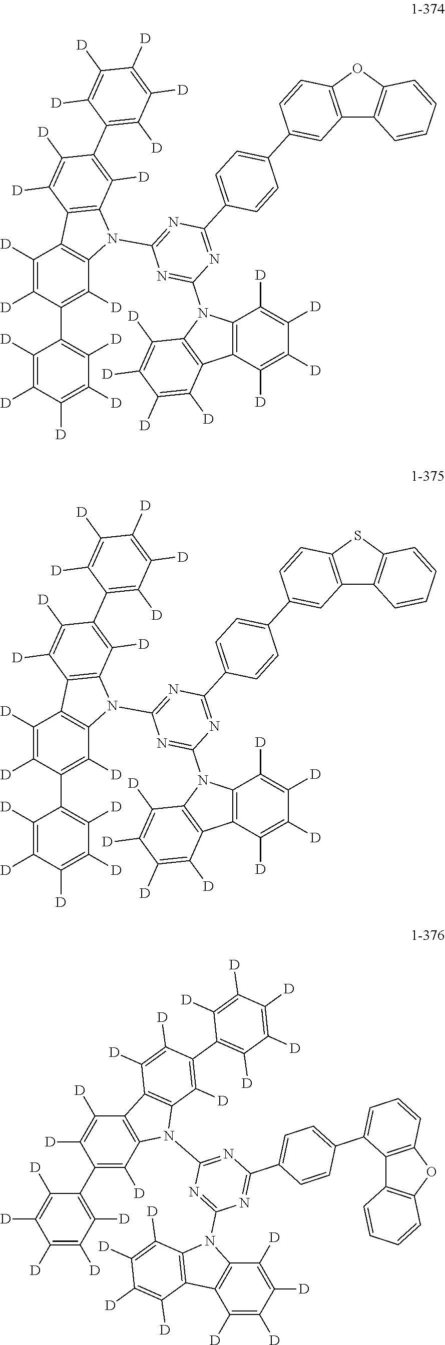

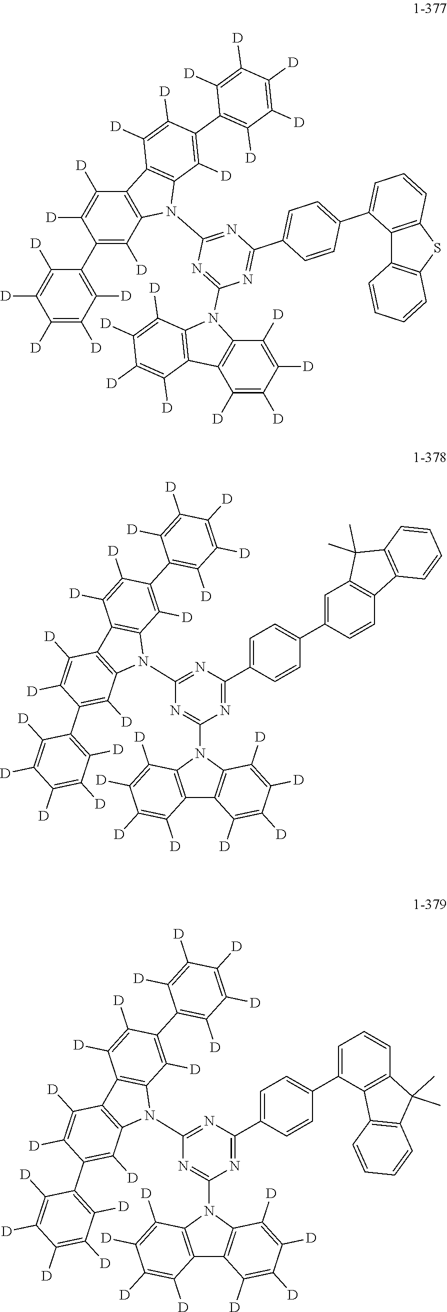

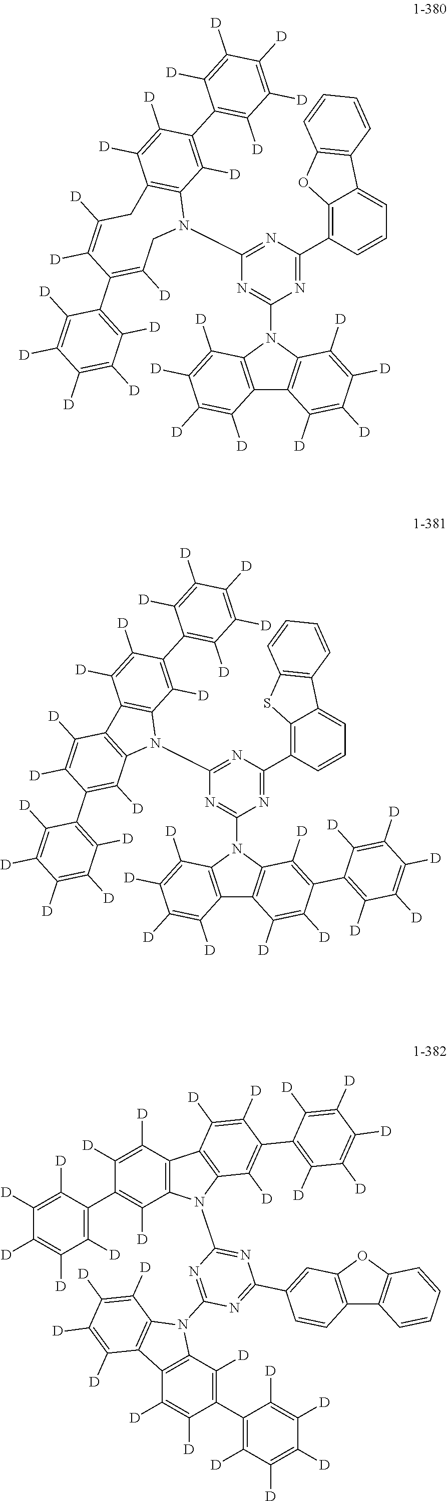

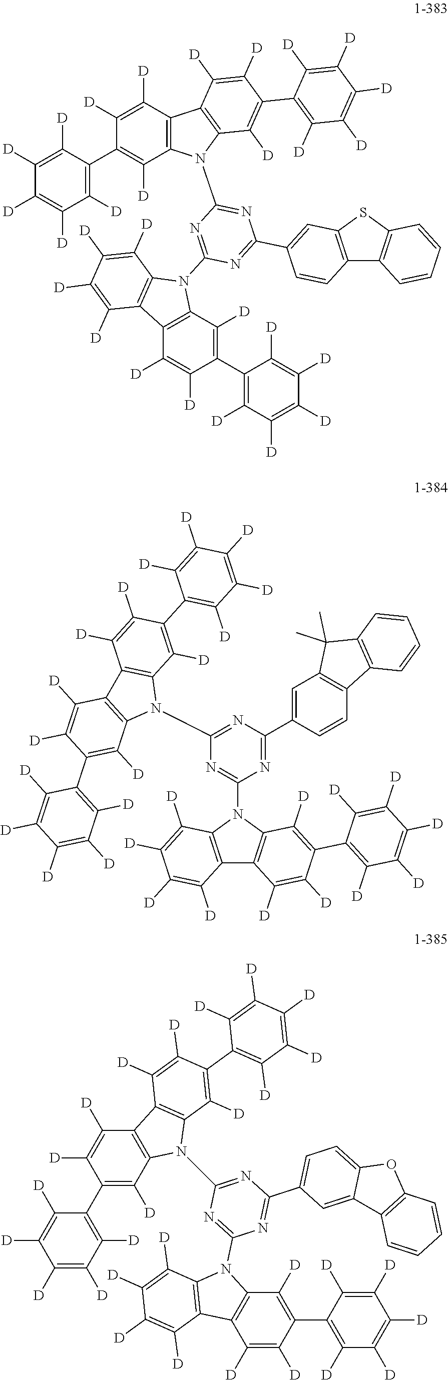

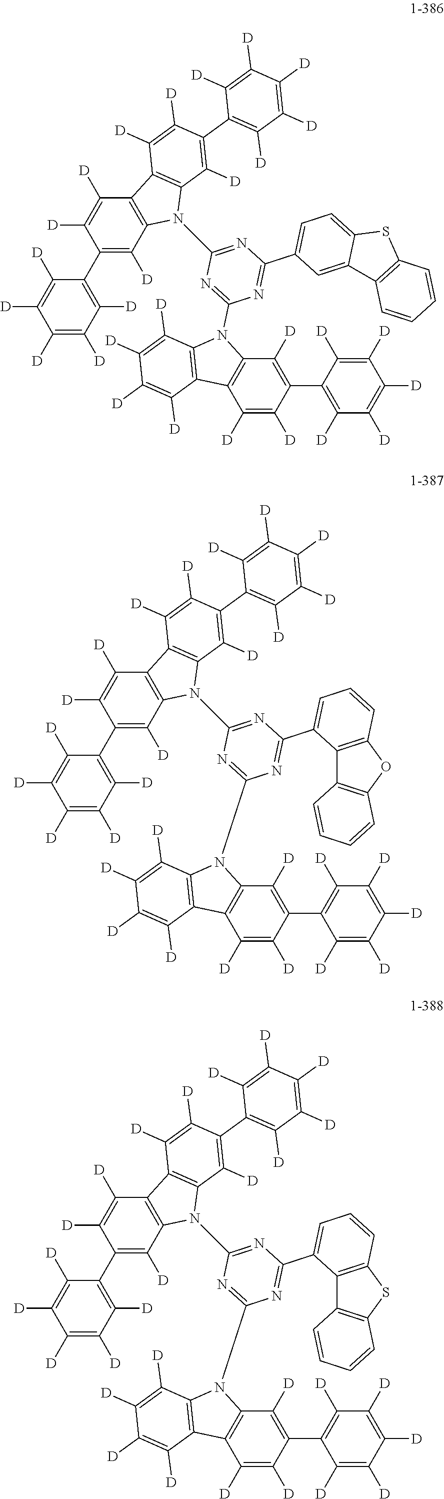

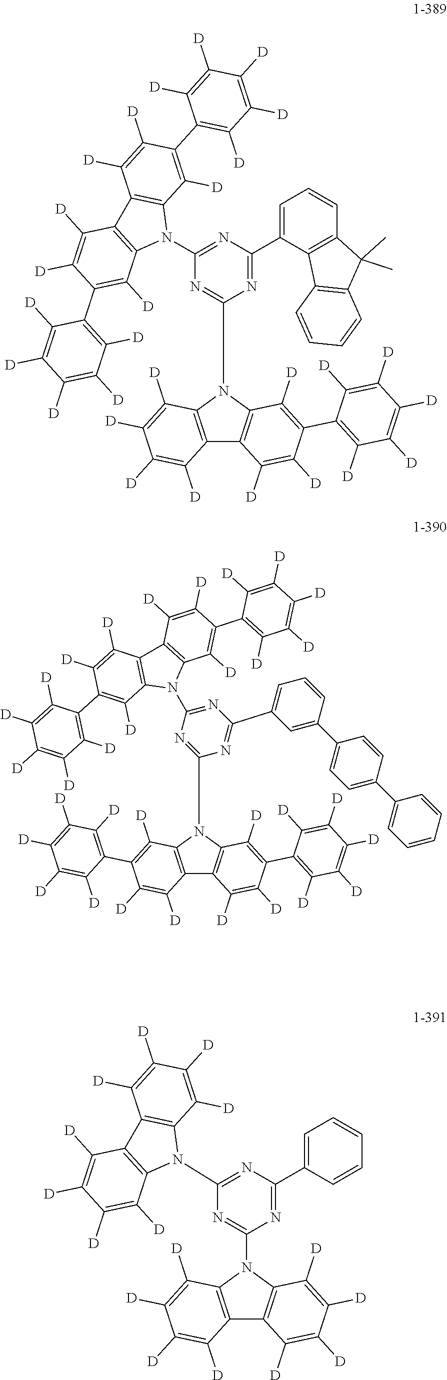

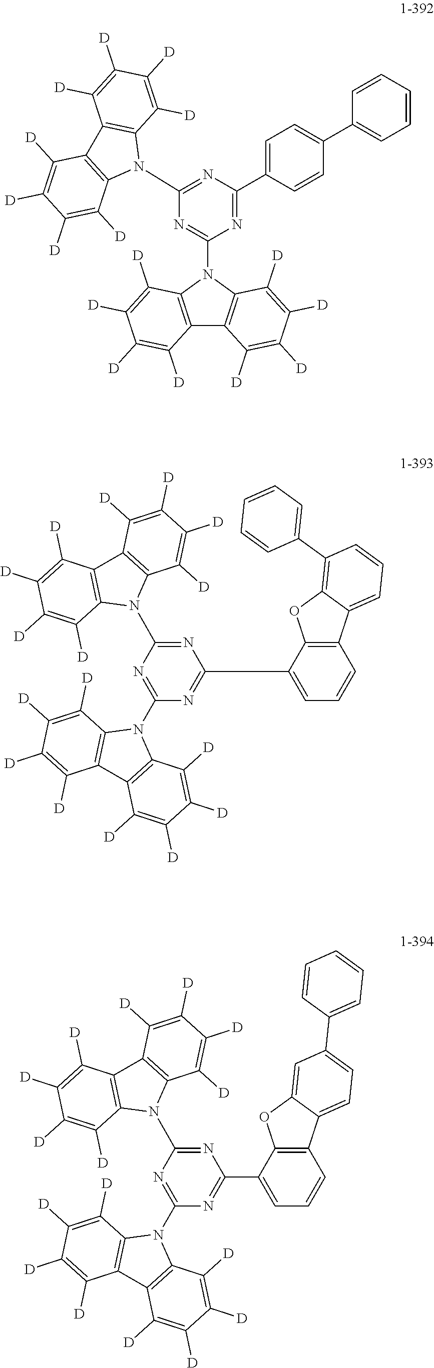

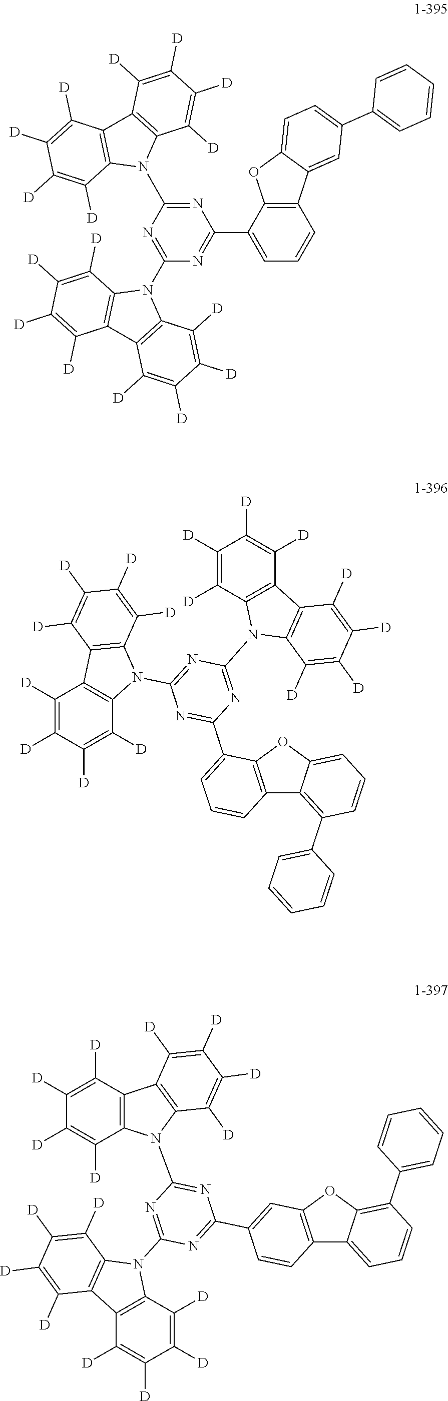

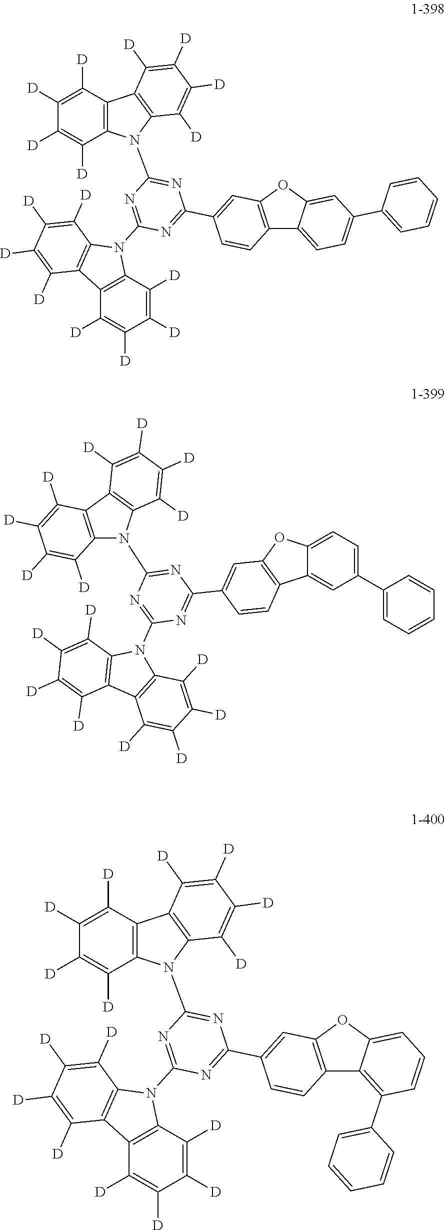

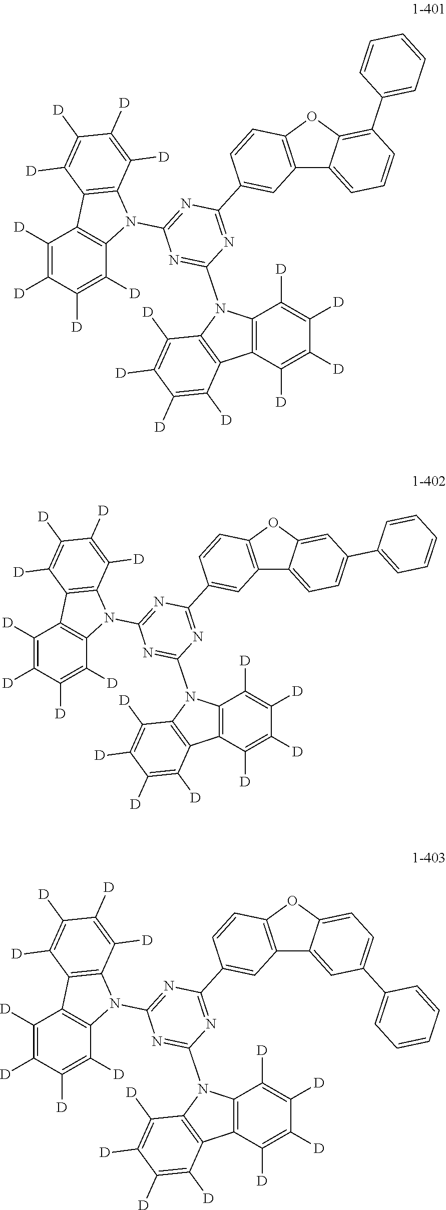

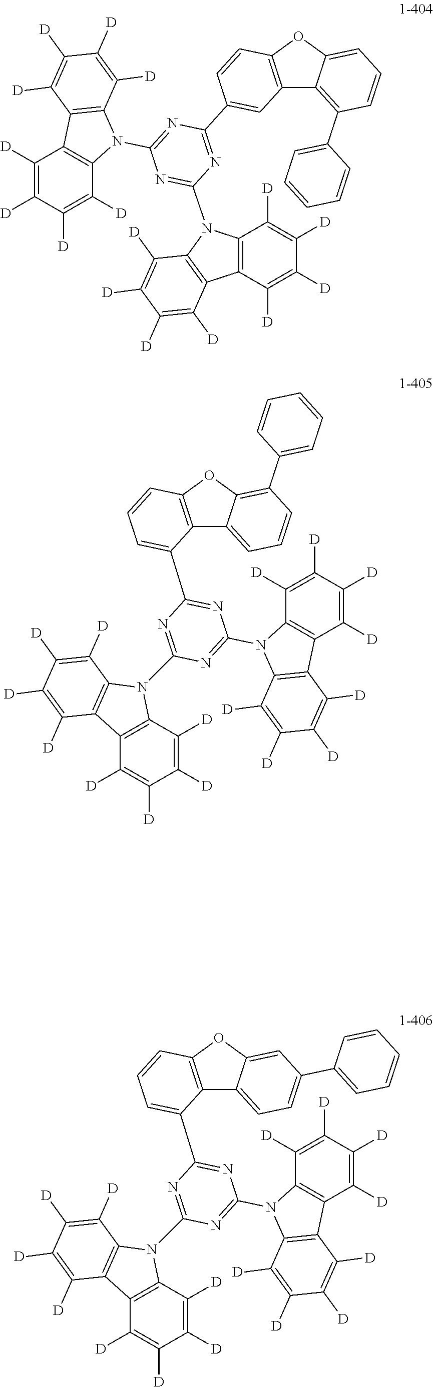

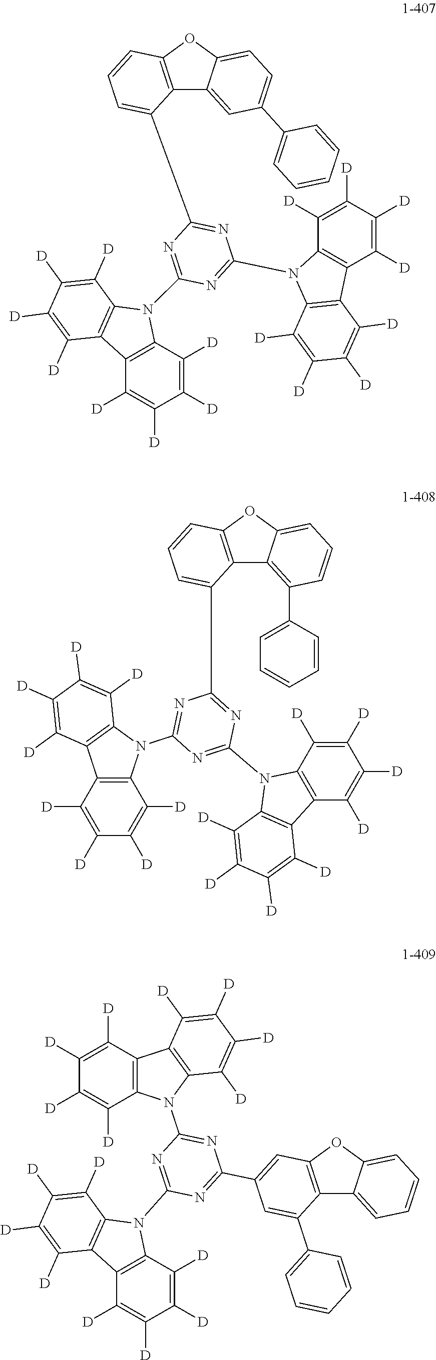

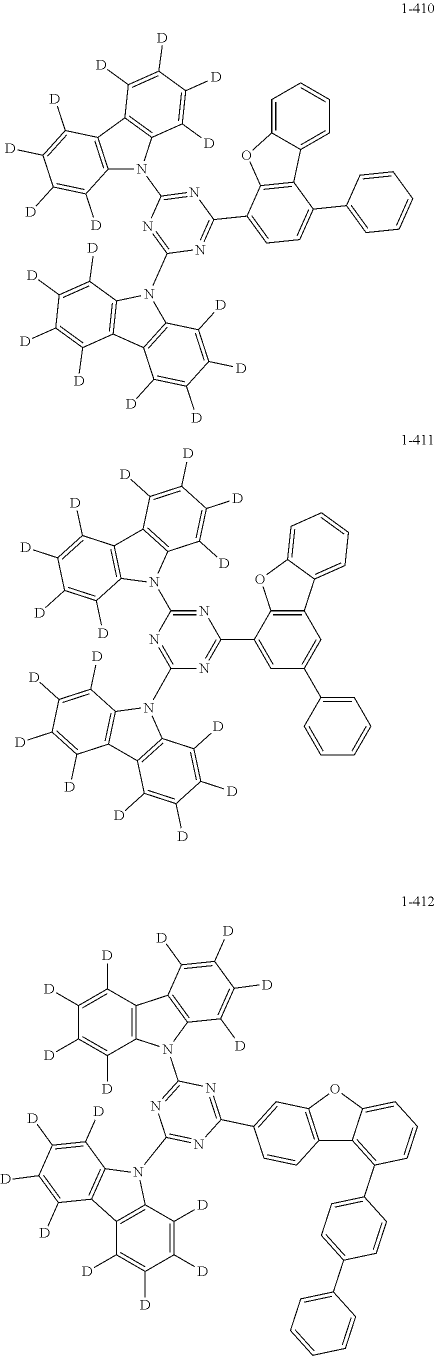

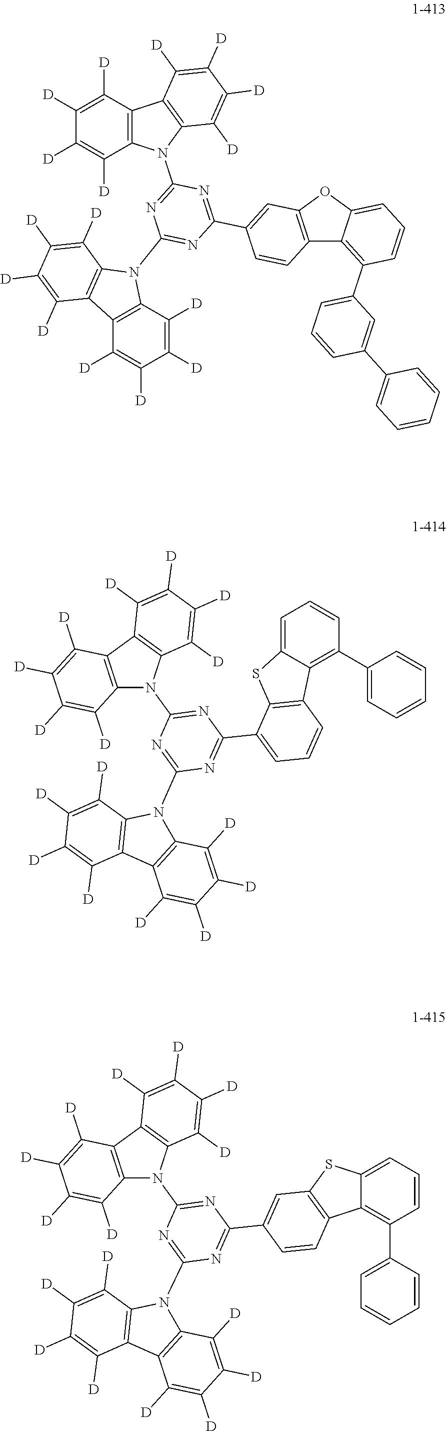

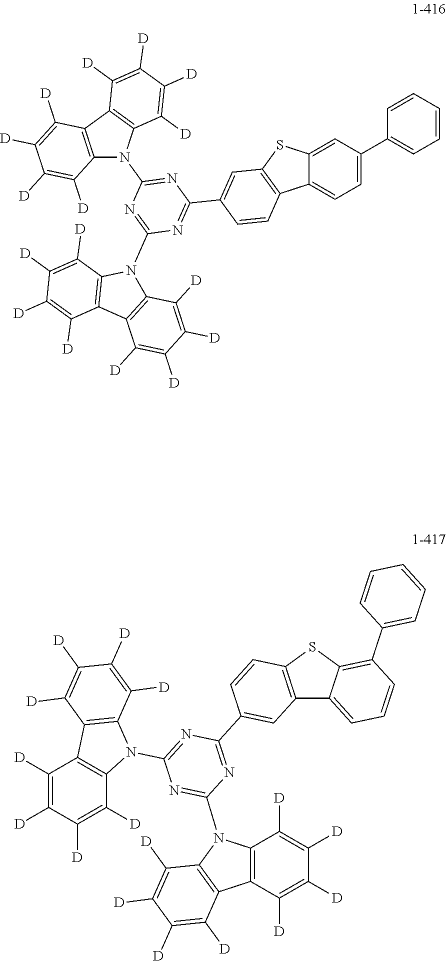 [SMILES: [2H]c1c([2H])c([2H])c2c(c1[2H])c1c([2H])c([2H])c([2H])c([2H])c1n2-c1nc(-c2ccc3c(c2)sc2cc(-c4ccccc4)ccc23)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2ccc3sc4c(-c5ccccc5)cccc4c3c2)nc(-n2c3c([2H])c([2H])c([2H])c([2H])c3c3c([2H])c([2H])c([2H])c([2H])c32)n1]

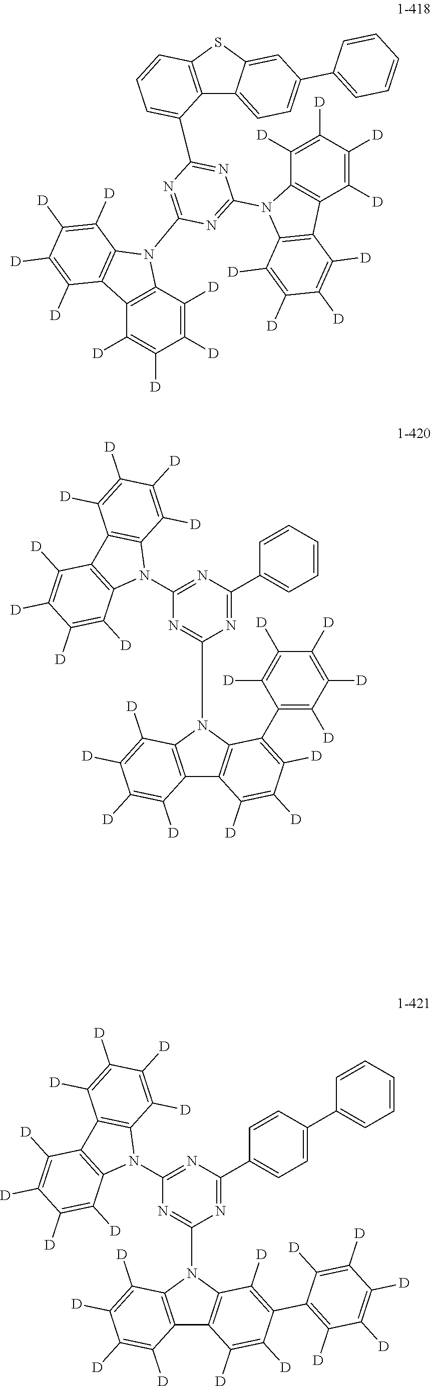

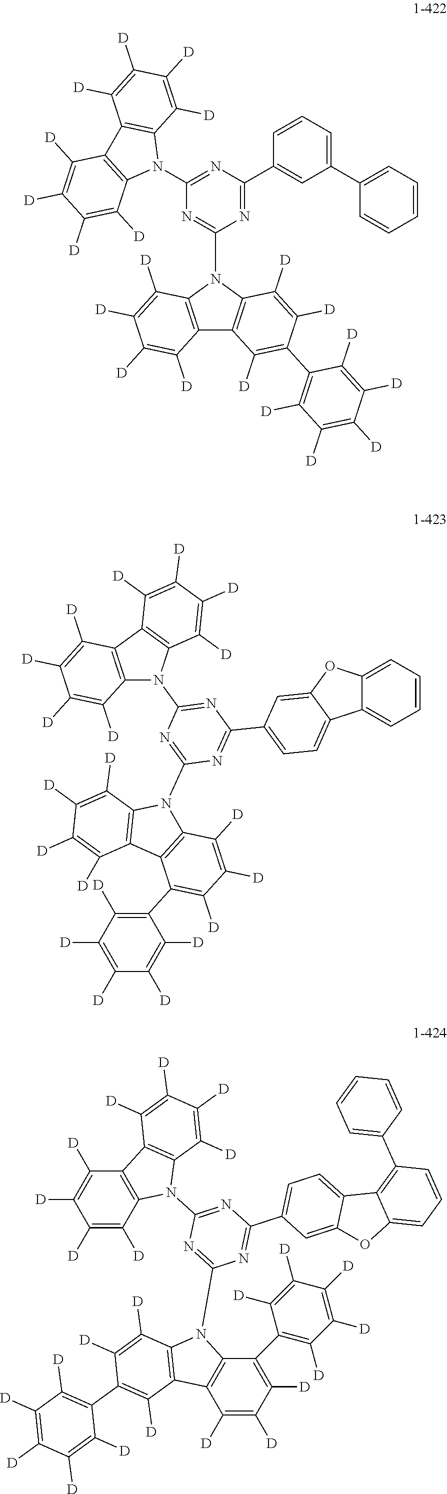

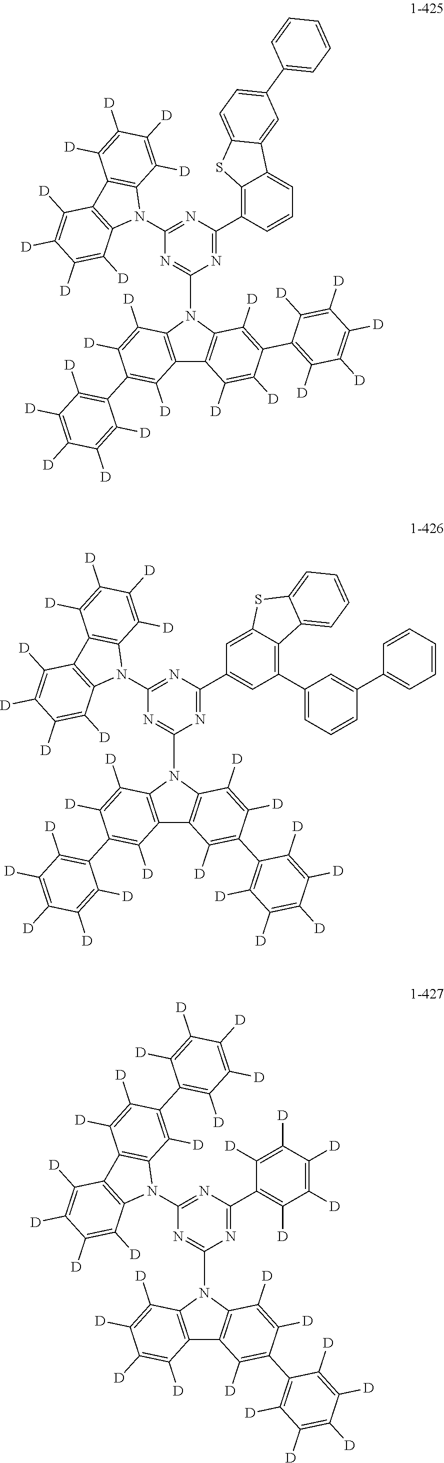

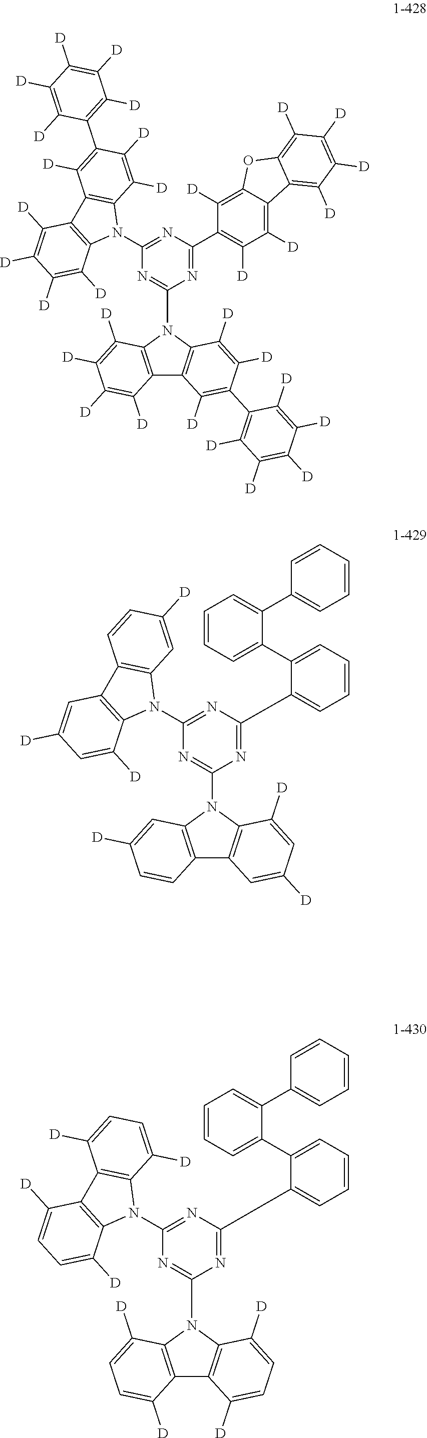

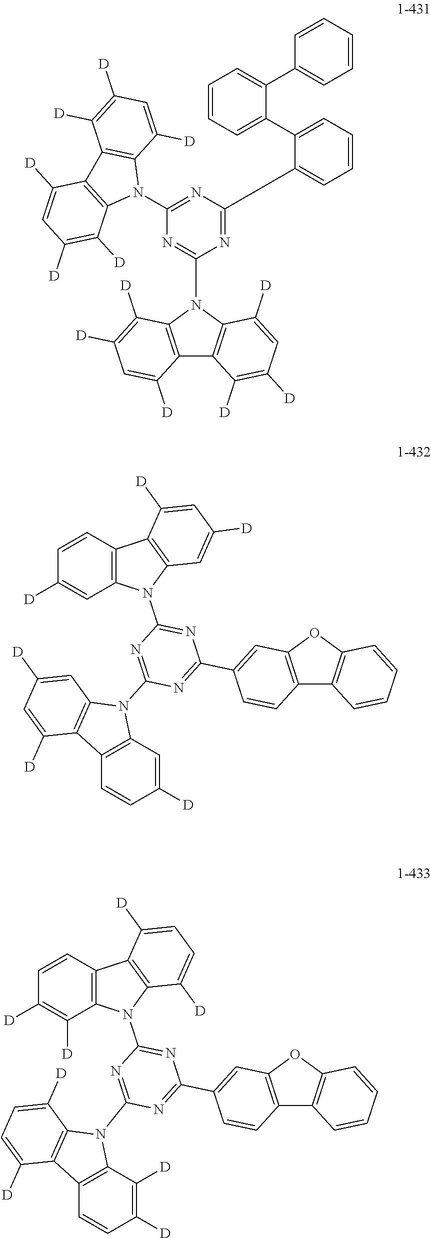 [SMILES: [2H]c1cc([2H])c2c(c1[2H])c1c([2H])cc([2H])c([2H])c1n2-c1nc(-c2ccccc2-c2ccccc2-c2ccccc2)nc(-n2c3c([2H])cc([2H])c([2H])c3c3c([2H])cc([2H])c([2H])c32)n1 ; [2H]c1ccc2c3c([2H])cc([2H])cc3n(-c3nc(-c4ccc5c(c4)oc4ccccc45)nc(-n4c5cc([2H])ccc5c5c([2H])cc([2H])cc54)n3)c2c1 ; [2H]c1ccc2c3c([2H])ccc([2H])c3n(-c3nc(-c4ccc5c(c4)oc4ccccc45)nc(-n4c5c([2H])c([2H])ccc5c5c([2H])ccc([2H])c54)n3)c2c1[2H]]

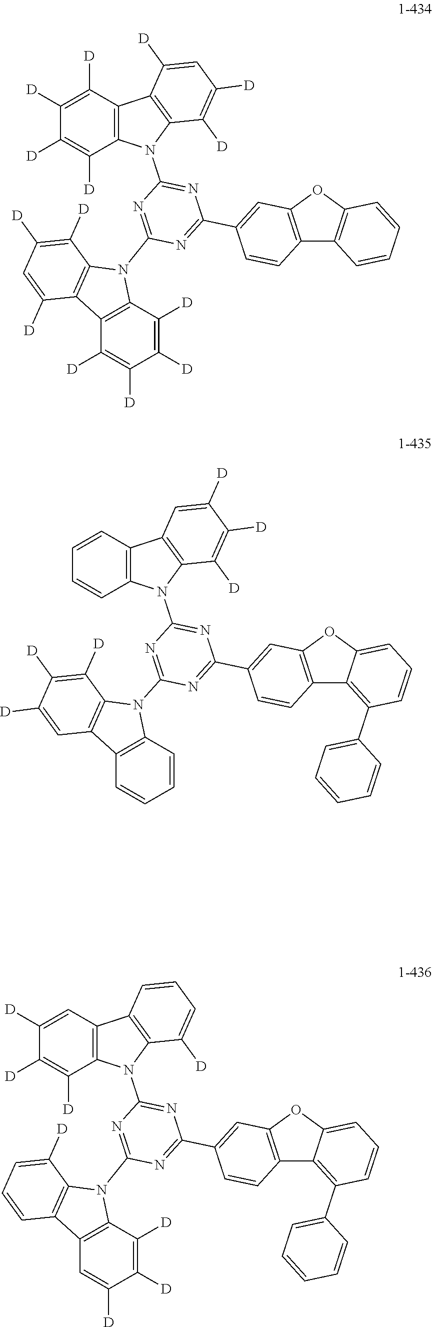 [SMILES: [2H]c1cc([2H])c2c3c([2H])c([2H])c([2H])c([2H])c3n(-c3nc(-c4ccc5c(c4)oc4ccccc45)nc(-n4c5c([2H])c([2H])cc([2H])c5c5c([2H])c([2H])c([2H])c([2H])c54)n3)c2c1[2H] ; [2H]c1cc2c3ccccc3n(-c3nc(-c4ccc5c(c4)oc4cccc(-c6ccccc6)c45)nc(-n4c5ccccc5c5cc([2H])c([2H])c([2H])c54)n3)c2c([2H])c1[2H] ; [2H]c1cc2c3cccc([2H])c3n(-c3nc(-c4ccc5c(c4)oc4cccc(-c6ccccc6)c45)nc(-n4c5c([2H])cccc5c5cc([2H])c([2H])c([2H])c54)n3)c2c([2H])c1[2H]]

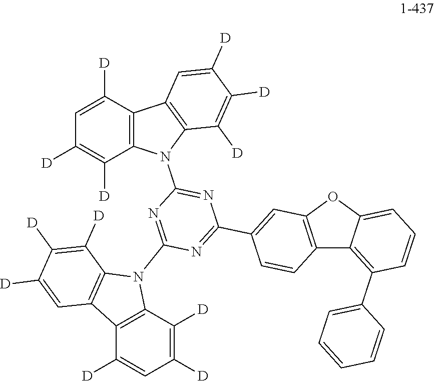 [SMILES: [2H]c1cc2c3c([2H])cc([2H])c([2H])c3n(-c3nc(-c4ccc5c(c4)oc4cccc(-c6ccccc6)c45)nc(-n4c5c([2H])c([2H])c([2H])cc5c5c([2H])cc([2H])c([2H])c54)n3)c2c([2H])c1[2H]]

Further, various substituents may be introduced into the structure of Chemical Formula 1 to synthesize a compound having inherent characteristics of a substituent introduced. For example, it is possible to synthesize a material which satisfies conditions required for each organic material layer by introducing a substituent usually used for a hole injection layer material, a material for transporting holes, a light emitting layer material, an electron transport layer material, and a charge generation layer material, which are used for preparing an organic light emitting device, into the core structure.

In addition, it is possible to finely adjust an energy band-gap by introducing various substituents into the structure of Chemical Formula 1, and meanwhile, it is possible to improve characteristics at the interface between organic materials and diversify the use of the material.

Meanwhile, the compound has a high glass transition temperature (Tg) and thus has excellent thermal stability. The increase in thermal stability becomes an important factor for providing a device with driving stability.

Further, in an exemplary embodiment of the present application, provided is an organic light emitting device including: a first electrode; a second electrode provided to face the first electrode; and an organic material layer having one or more layers provided between the first electrode and the second electrode, in which one or more layers of the organic material layer include the heterocyclic compound represented by Chemical Formula 1.

In an exemplary embodiment of the present application, the first electrode may be a positive electrode, and the second electrode may be a negative electrode.

In another exemplary embodiment, the first electrode may be a negative electrode, and the second electrode may be a positive electrode.

In an exemplary embodiment of the present application, the organic light emitting device may be a blue organic light emitting device, and the compound represented by Chemical Formula 1 may be used as a material for the blue organic light emitting device.

In an exemplary embodiment of the present application, the organic light emitting device may be a green organic light emitting device, and the compound represented by Chemical Formula 1 may be used as a material for the green organic light emitting device.

In an exemplary embodiment of the present application, the organic light emitting device may be a red organic light emitting device, and the compound represented by Chemical Formula 1 may be used as a material for the red organic light emitting device.

In an exemplary embodiment of the present application, the organic light emitting device may be a blue organic light emitting device, and the compound represented by Chemical Formula 1 may be used as a material for a light emitting layer of the blue organic light emitting device.

In an exemplary embodiment of the present application, the organic light emitting device may be a green organic light emitting device, and the compound represented by Chemical Formula 1 may be used as a material for a light emitting layer of the green organic light emitting device.

In an exemplary embodiment of the present application, the organic light emitting device may be a red organic light emitting device, and the compound represented by Chemical Formula 1 may be used as a material for a light emitting layer of the red organic light emitting device.

The specific content on the heterocyclic compound represented by Chemical Formula 1 is the same as that described above.

The organic light emitting device of the present invention may be manufactured using typical manufacturing methods and materials of an organic light emitting device, except that the above-described heterocyclic compound is used to form an organic material layer having one or more layers.

The heterocyclic compound may be formed as an organic material layer by not only a vacuum deposition method, but also a solution application method when an organic light emitting device is manufactured. Herein, the solution coating method means spin coating, dip coating, inkjet printing, screen printing, a spray method, roll coating, and the like, but is not limited thereto.

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In the organic light emitting device of the present invention, the organic material layer includes a light emitting layer, and the light emitting layer may include the heterocyclic compound.

In an exemplary embodiment of the present application, provided is an organic light emitting device, in which the organic material layer includes a light emitting layer, the light emitting layer includes a host material, and the host material includes the heterocyclic compound.

An exemplary embodiment of the present application provides an organic light emitting device in which an organic material layer including the heterocyclic compound of Chemical Formula 1 further includes a heterocyclic compound represented by the following Chemical Formula 2 or 3.

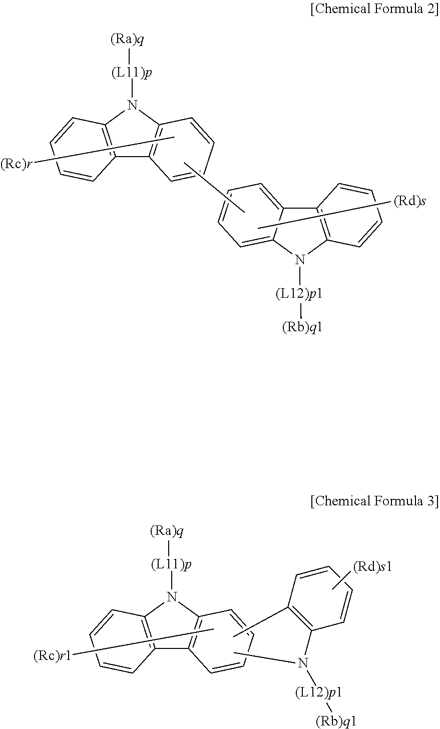

In Chemical Formulae 2 and 3,
Rc and Rd are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group,
L11 and L12 are the same as or different from each other, and are each independently a direct bond; a substituted or unsubstituted C6 to C60 arylene group; or a substituted or unsubstituted C2 to C60 heteroarylene group,
Ra and Rb are the same as or different from each other, and are each independently a substituted or unsubstituted C6 to C60 aryl group; a substituted or unsubstituted C2 to C60 heteroaryl group; a cyano group; or —SiRR′R″,
R, R″, and R″ are the same as or different from each other, and are each independently hydrogen; deuterium; —CN; a substituted or unsubstituted C1 to C60 alkyl group; a substituted or unsubstituted C6 to C60 aryl group; or a substituted or unsubstituted C2 to C60 heteroaryl group,
p, p1, q, and q1 are an integer from 1 to 4,
r and s are an integer from 0 to 7,
r1 is an integer from 0 to 6,
s1 is an integer from 0 to 4, and
when p, p1, q, q1, r, s, r1, and s1 are 2 or higher, substituent in the parenthesis are the same as or different from each other.

When the heterocyclic compound of Chemical Formula 1 and the heterocyclic compound of Chemical Formula 2 or 3 are included in the organic material layer of the organic light emitting device, better efficiency and service life effects are exhibited. From this result, it can be expected that an exciplex phenomenon will occur when both compounds are included.

The exciplex phenomenon is a phenomenon in which energy with a magnitude of the HOMO level of a donor (p-host) and the LUMO level of an acceptor (n-host) is released due to an electron exchange between two molecules. When the exciplex phenomenon between two molecules occurs, a reverse intersystem crossing (RISC) occurs, and the internal quantum efficiency of fluorescence can be increased to 100% due to the RISC. When a donor with a good hole transport capacity (p-host) and an acceptor with a good electron transport capacity (n-host) are used as hosts for the light emitting layer, holes are injected into the p-host and electrons are injected into the n-host, so that the driving voltage can be lowered, which can help to improve the service life.

In an exemplary embodiment of the present application, Rc and Rd are the same as or different from each other, and may be each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group.

In another exemplary embodiment, Rc and Rd are the same as or different from each other, and may be each independently selected from the group consisting of hydrogen; deuterium; a halogen; a cyano group; a substituted or unsubstituted C1 to C40 alkyl group; a substituted or unsubstituted C2 to C40 alkenyl group; a substituted or unsubstituted C2 to C40 alkynyl group; a substituted or unsubstituted C1 to C40 alkoxy group; a substituted or unsubstituted C3 to C40 cycloalkyl group; a substituted or unsubstituted C2 to C40 heterocycloalkyl group; a substituted or unsubstituted C6 to C40 aryl group; and a substituted or unsubstituted C2 to C40 heteroaryl group.

In still another exemplary embodiment, Rc and Rd are the same as or different from each other, and may be each independently selected from the group consisting of hydrogen; deuterium; a substituted or unsubstituted C1 to C40 alkyl group; a substituted or unsubstituted C6 to C40 aryl group; and a substituted or unsubstituted C2 to C40 heteroaryl group.

In yet another exemplary embodiment, Rc and Rd are the same as or different from each other, and may be each independently selected from the group consisting of hydrogen; deuterium; a substituted or unsubstituted C1 to C20 alkyl group; a substituted or unsubstituted C6 to C20 aryl group; or a substituted or unsubstituted C2 to C20 heteroaryl group.

In still yet another exemplary embodiment, Rc and Rd are the same as or different from each other, and may be each independently hydrogen; or deuterium.

In an exemplary embodiment of the present application, L11 and L12 are the same as or different from each other, and are each independently a direct bond; a substituted or unsubstituted C6 to C60 arylene group; or a substituted or unsubstituted C2 to C60 heteroarylene group.

In another exemplary embodiment, L11 and L12 are the same as or different from each other, and are each independently a direct bond; a substituted or unsubstituted C6 to C40 arylene group; or a substituted or unsubstituted C2 to C40 heteroarylene group.

In still another exemplary embodiment, L11 and L12 are the same as or different from each other, and are each independently a direct bond; a substituted or unsubstituted C6 to C20 arylene group; or a substituted or unsubstituted C2 to C20 heteroarylene group.

In yet another exemplary embodiment, L11 and L12 are the same as or different from each other, and are each independently a direct bond; or a substituted or unsubstituted C6 to C20 arylene group.

In still yet another exemplary embodiment, L11 and L12 are the same as or different from each other, and are each independently a direct bond; or a C6 to C20 arylene group unsubstituted or substituted with deuterium.

In a further exemplary embodiment, L11 and L12 are the same as or different from each other, and may be each independently a direct bond; a substituted or unsubstituted phenylene group; a substituted or unsubstituted biphenylene group; or a substituted or unsubstituted naphthylene group.

In another further exemplary embodiment, L11 and L12 are the same as or different from each other, and may be each independently a direct bond; a phenylene group; a biphenylene group; or a naphthylene group.

L11 and L12 may be unsubstituted or substituted with deuterium.

In an exemplary embodiment of the present application, Ra and Rb are the same as or different from each other, and are each independently a substituted or unsubstituted C6 to C60 aryl group; a substituted or unsubstituted C2 to C60 heteroaryl group; a cyano group; or —SiRR′R″.

In another exemplary embodiment, Ra and Rb are the same as or different from each other, and are each independently a substituted or unsubstituted C6 to C40 aryl group; a substituted or unsubstituted C2 to C40 heteroaryl group; a cyano group; or —SiRR′R″.

In still another exemplary embodiment, Ra and Rb are the same as or different from each other, and are each independently a substituted or unsubstituted C6 to C20 aryl group; a substituted or unsubstituted C2 to C20 heteroaryl group; a cyano group; or —SiRR′R″.

In yet another exemplary embodiment, Ra and Rb are the same as or different from each other, and may be each independently a substituted or unsubstituted phenyl group; a substituted or unsubstituted biphenyl group; a substituted or unsubstituted naphthyl group; —SiRR′R″; a substituted or unsubstituted terphenyl group; a substituted or unsubstituted triphenylenyl group; a cyano group; a substituted or unsubstituted dimethylfluorenyl group; a substituted or unsubstituted diphenylfluorenyl group; a substituted or unsubstituted spirobifluorenyl group; a substituted or unsubstituted dibenzothiophene group; or a substituted or unsubstituted dibenzofuran group.

In an exemplary embodiment of the present application, Chemical Formula 2 may be represented by the following Chemical Formula 2-1.

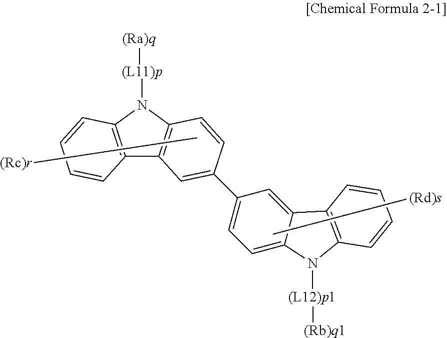

In Chemical Formula 2-1,
the definition of each substituent is the same as the definition in Chemical Formula 2.

In an exemplary embodiment of the present application, Chemical Formula 3 may be represented by any one of the following Chemical Formulae 3-1 to 3-6.

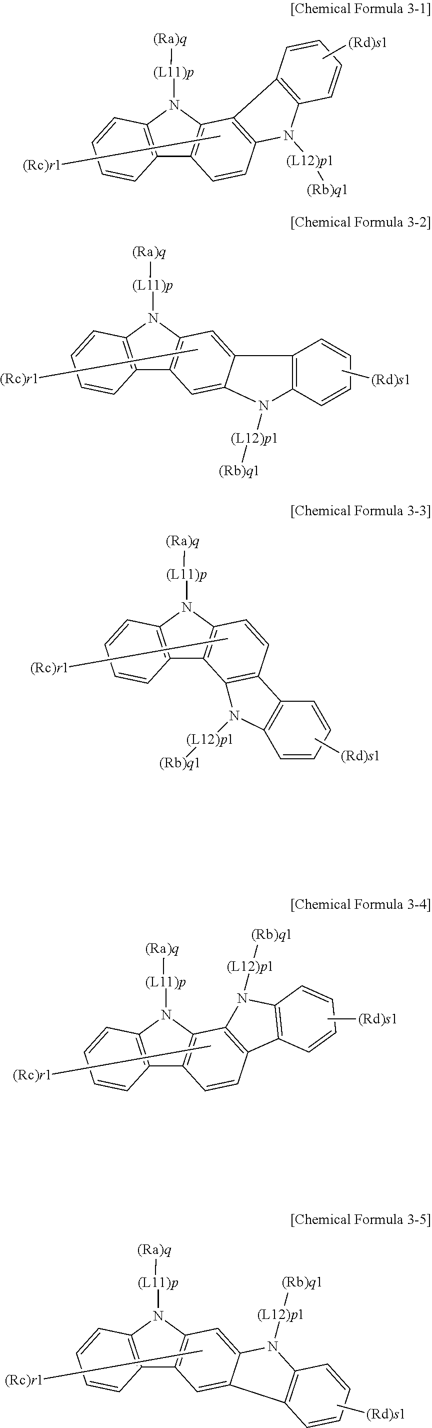

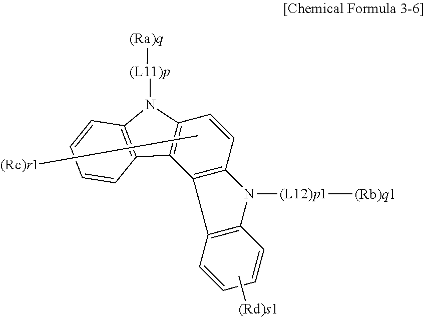

In Chemical Formulae 3-1 to 3-6,
the definition of each substituent is the same as the definition in Chemical Formula 3.

In an exemplary embodiment of the present application, R, R′, and R″ are the same as or different from each other, and are each independently hydrogen; deuterium; —CN; a substituted or unsubstituted C1 to C60 alkyl group; a substituted or unsubstituted C6 to C60 aryl group; or a substituted or unsubstituted C2 to C60 heteroaryl group.

In another exemplary embodiment, R, R′, and R″ are the same as or different from each other, and may be each independently a substituted or unsubstituted C1 to C60 alkyl group; or a substituted or unsubstituted C6 to C60 aryl group.

In still another exemplary embodiment, R, R′, and R″ are the same as or different from each other, and may be each independently a C1 to C60 alkyl group; or a C6 to C60 aryl group.

In yet another exemplary embodiment, R, R′, and R″ are the same as or different from each other, and may be each independently a methyl group; or a phenyl group.

In still yet another exemplary embodiment, R, R′, and R″ may be a phenyl group.

In an exemplary embodiment of the present application, the heterocyclic compound represented by Chemical Formula 2 or 3 may be any one selected among the following compounds.

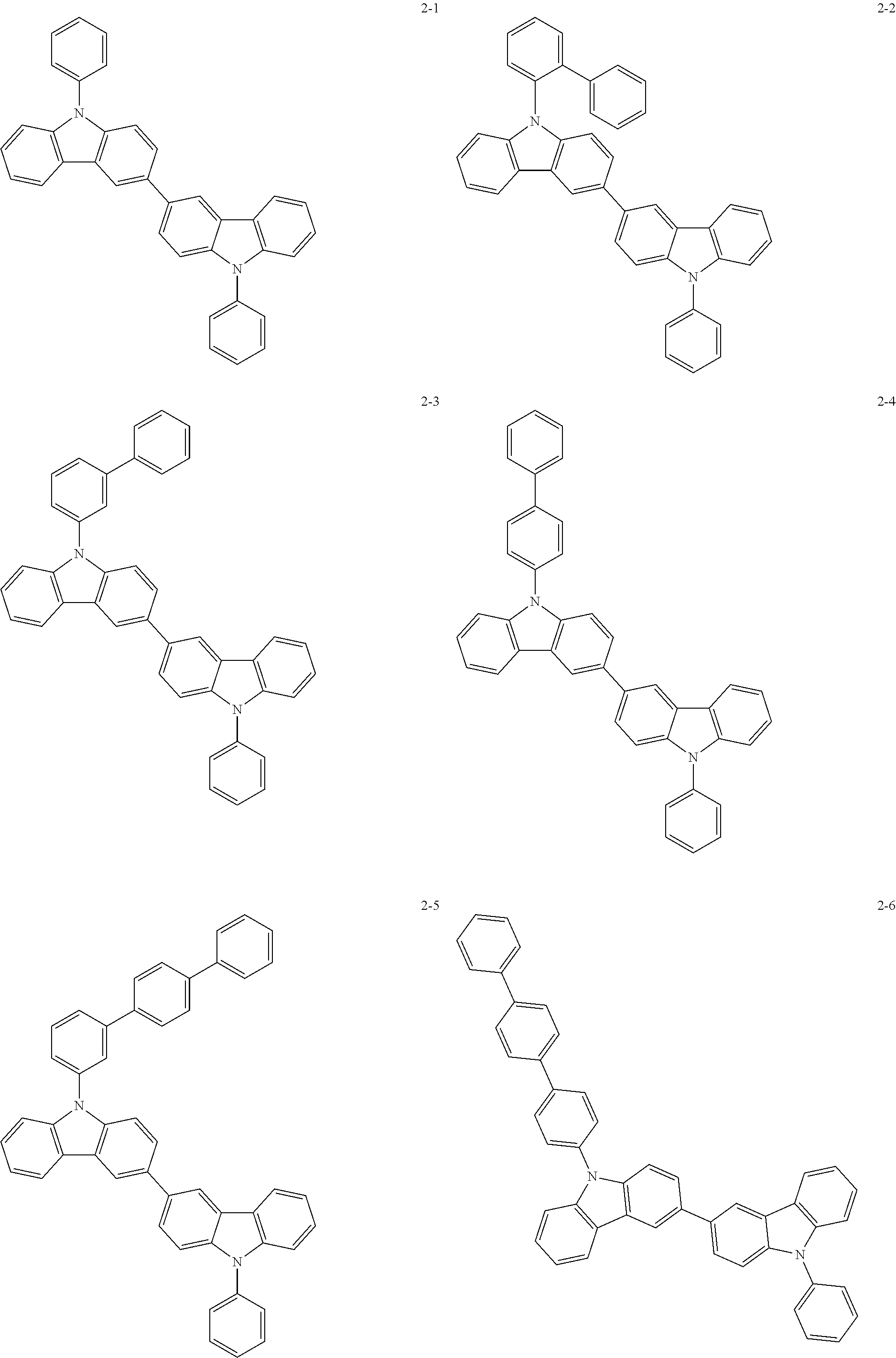 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

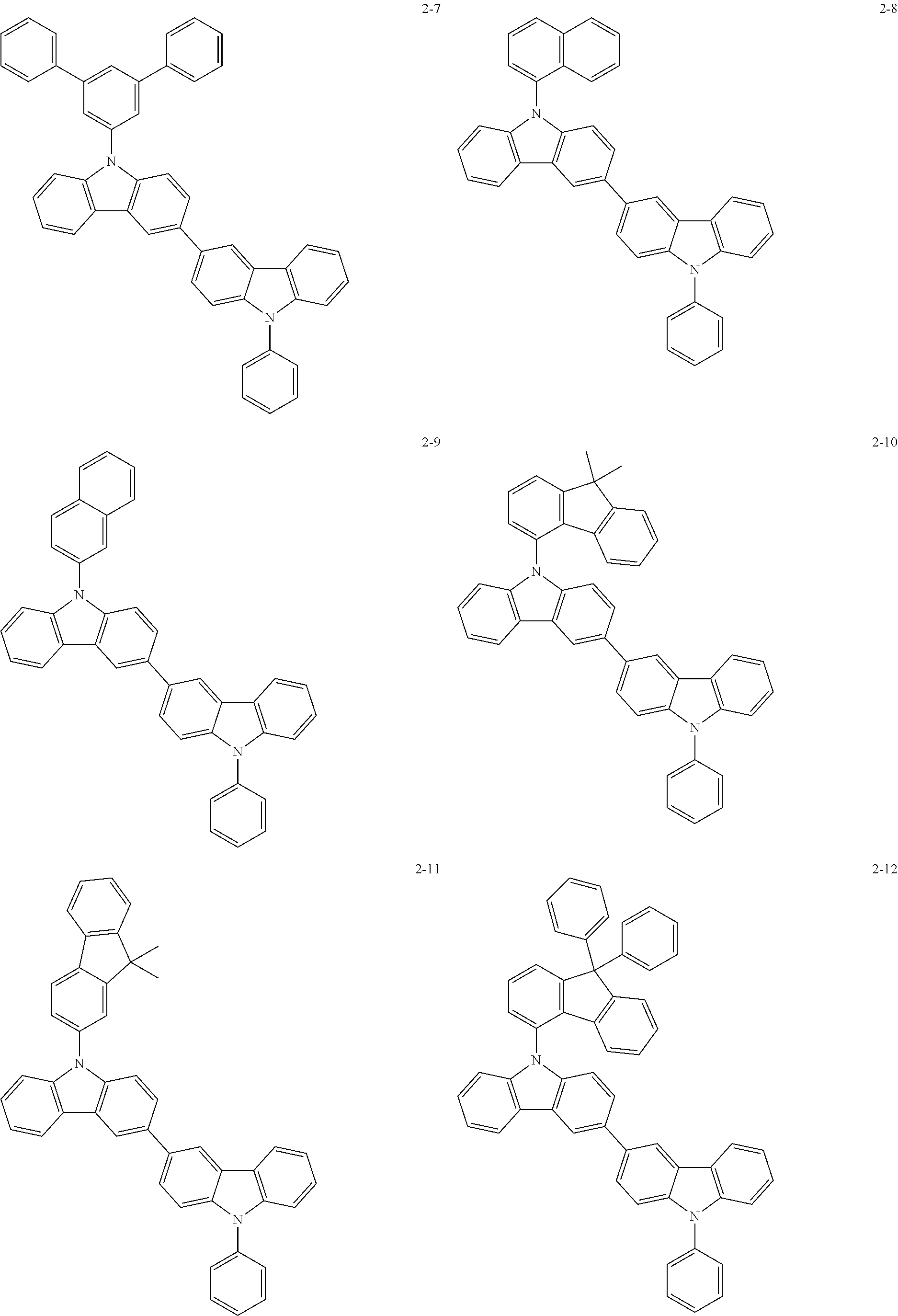 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

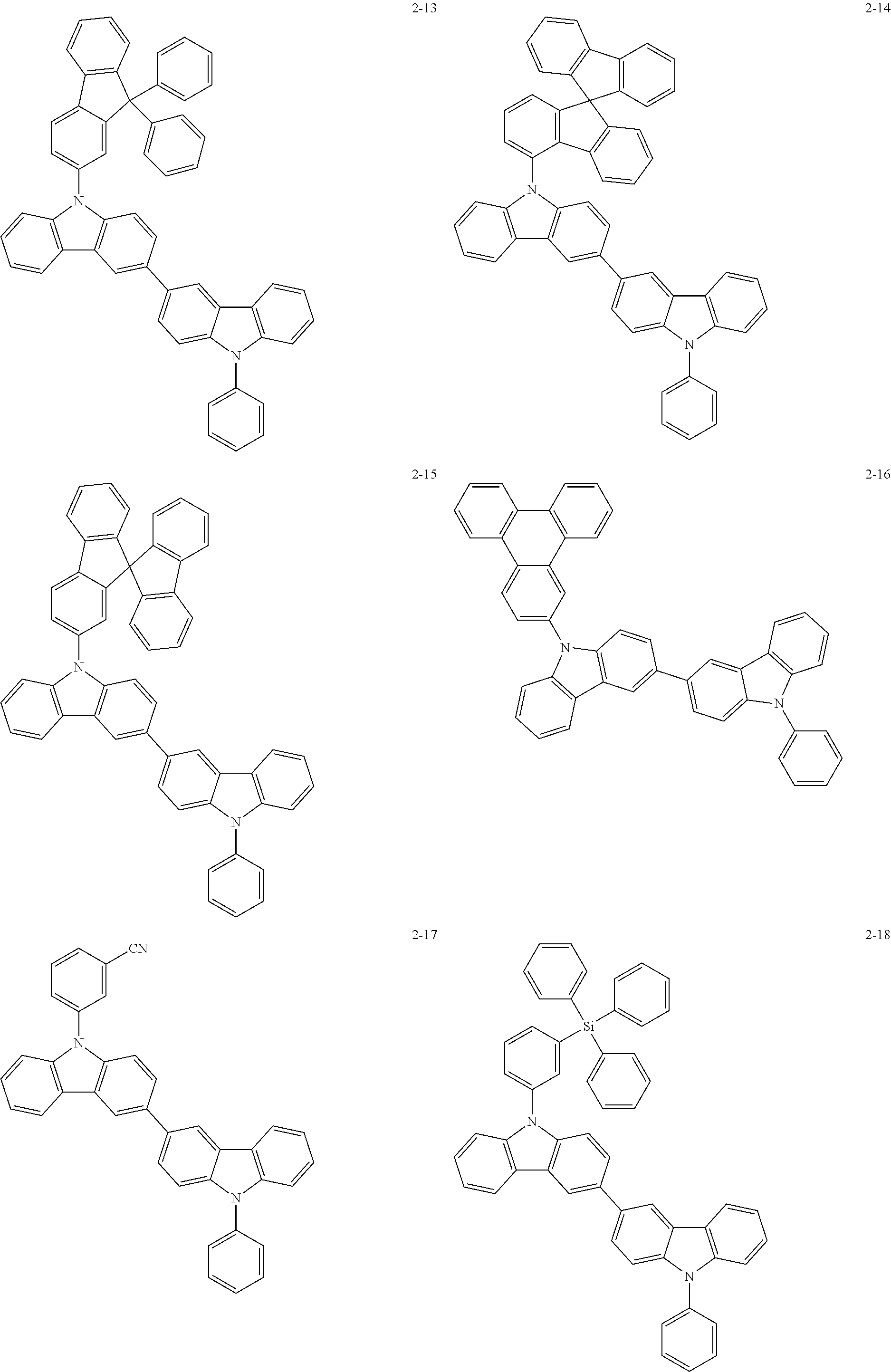

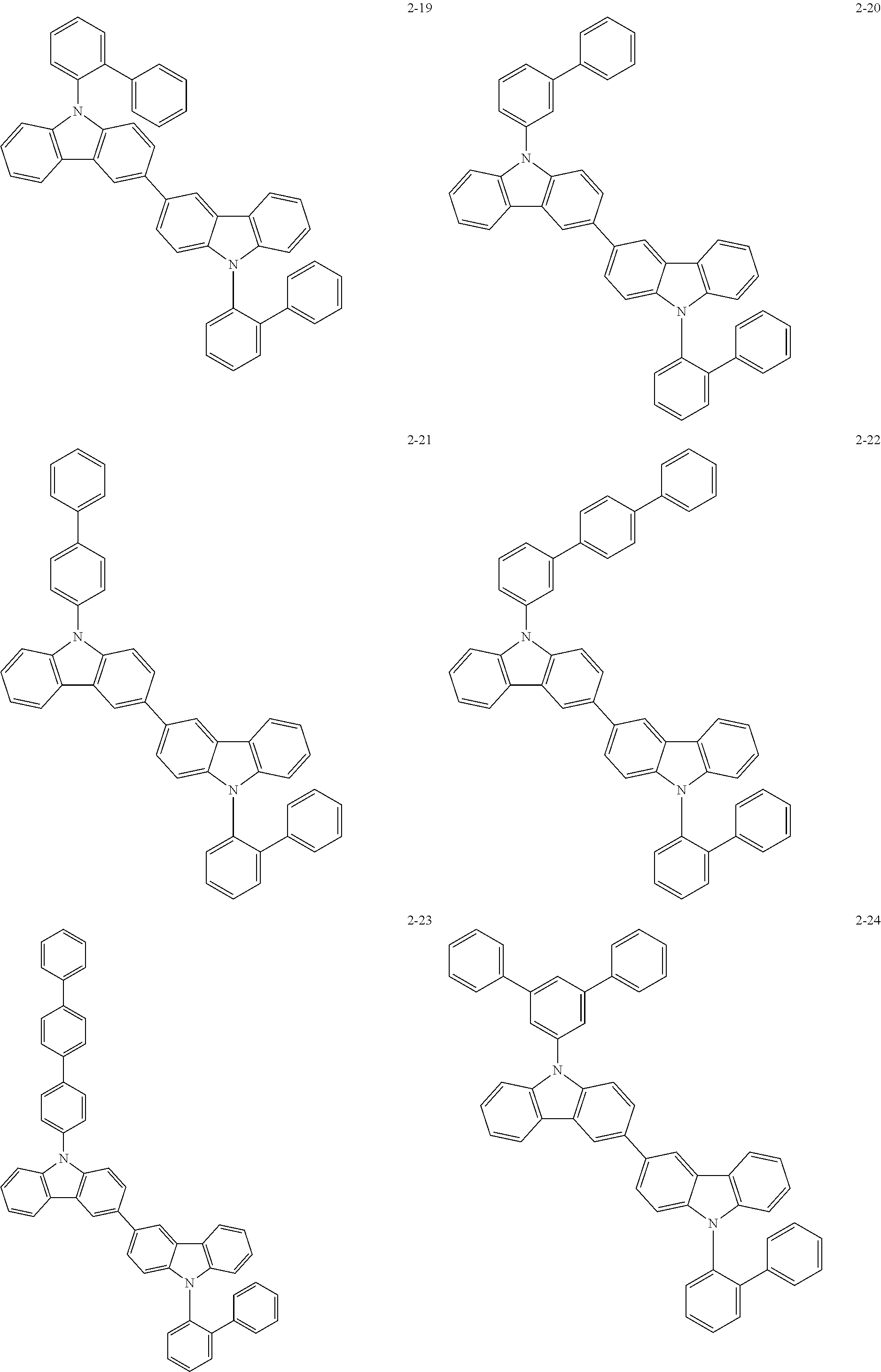

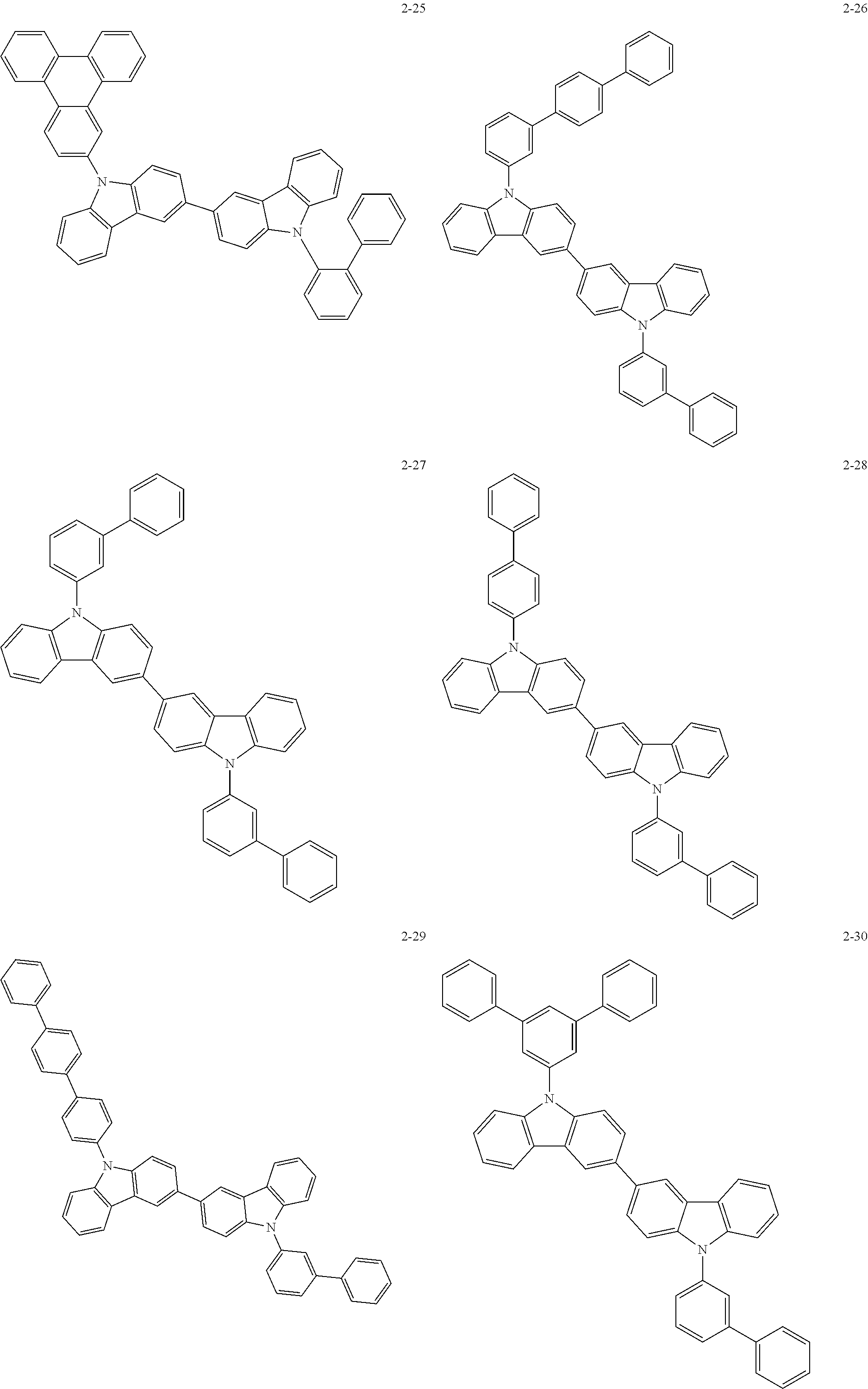

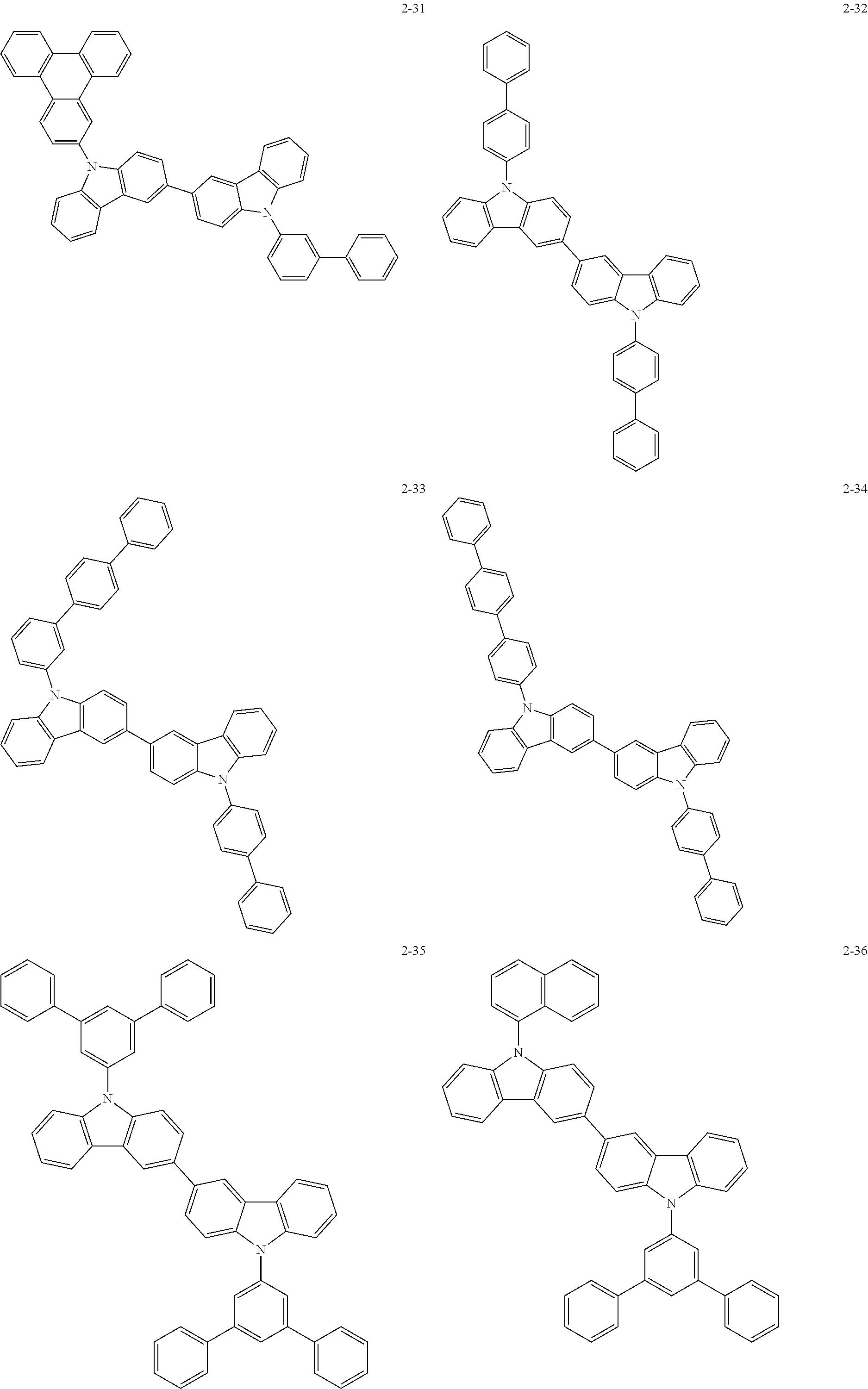

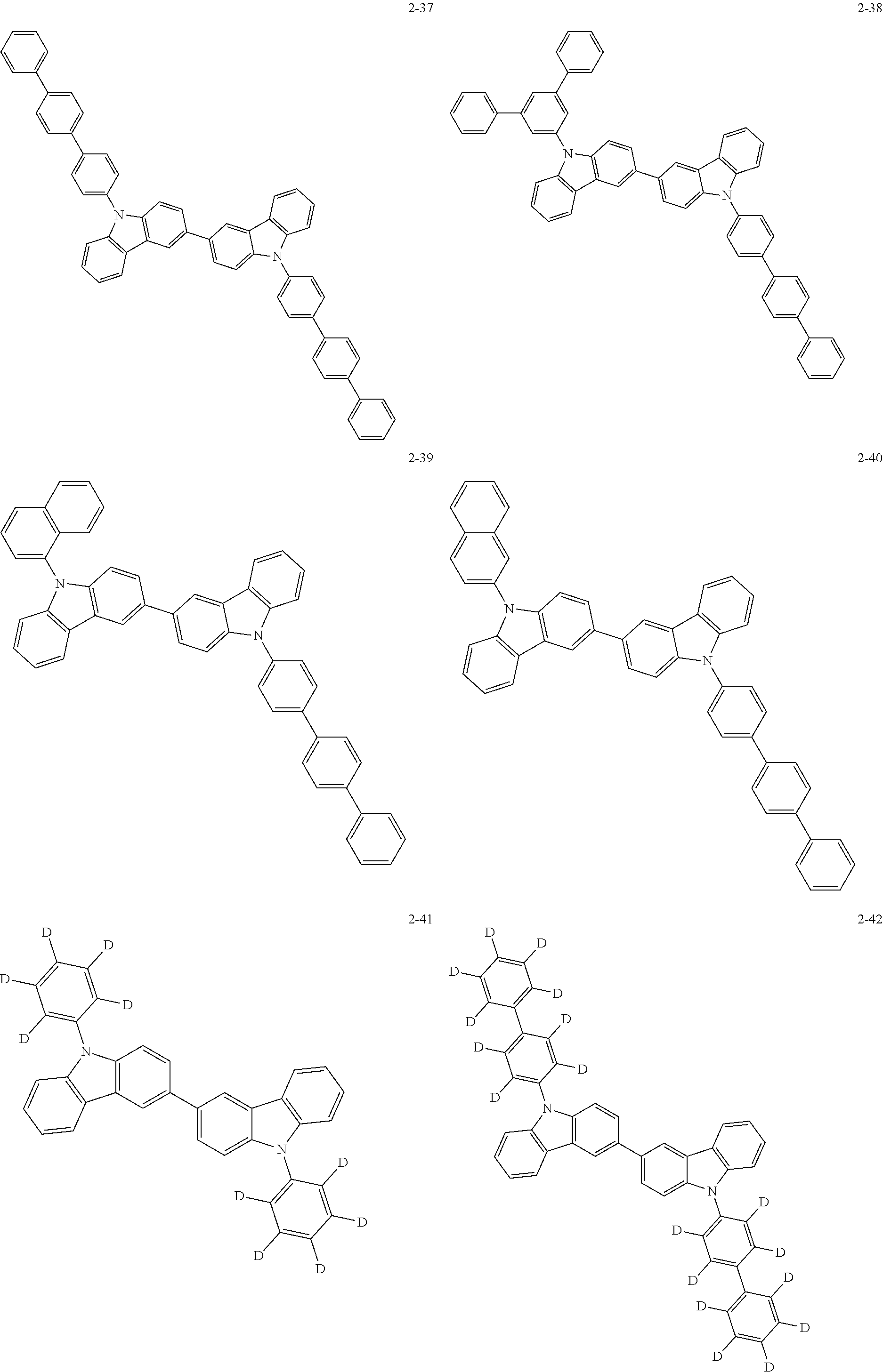

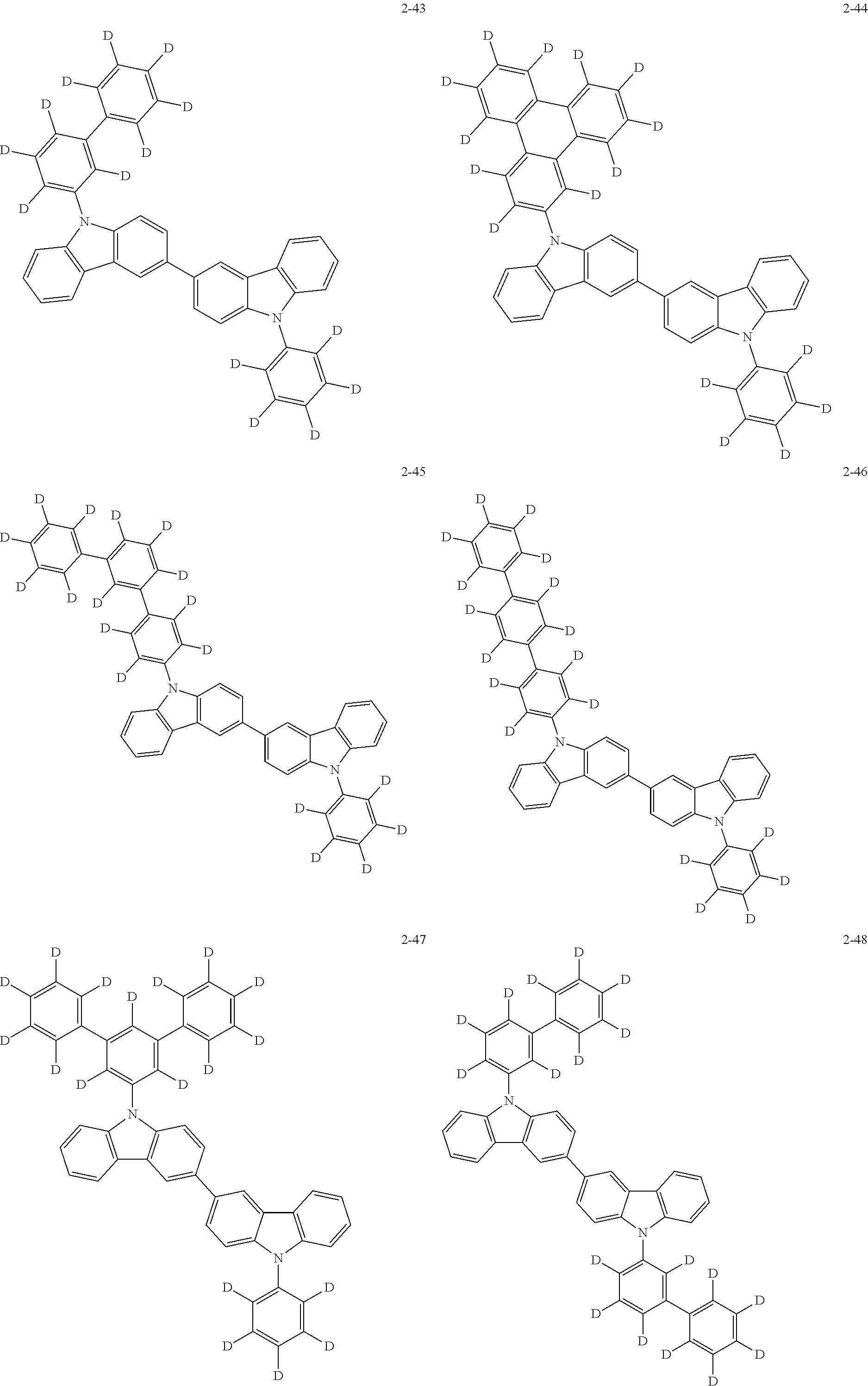

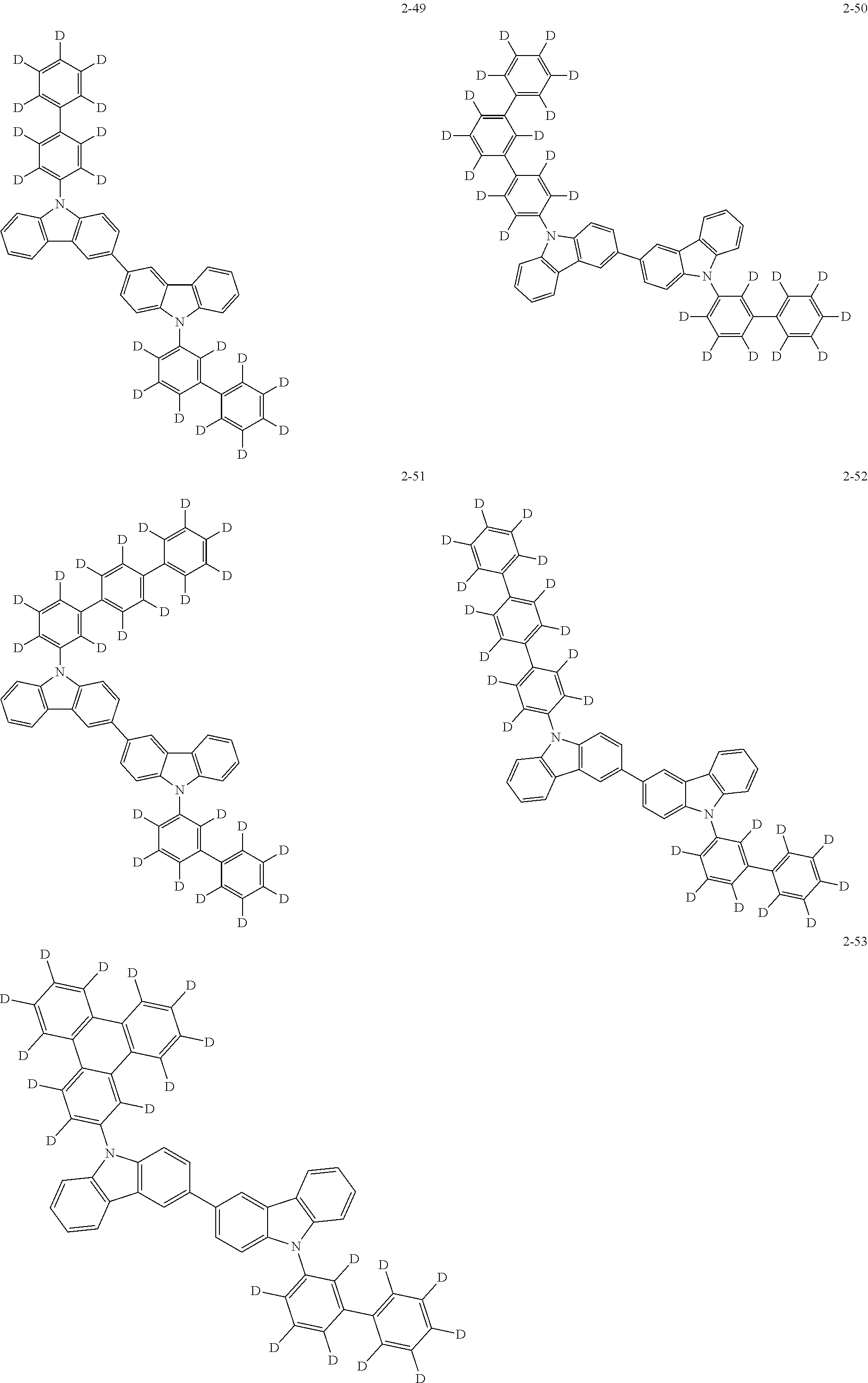

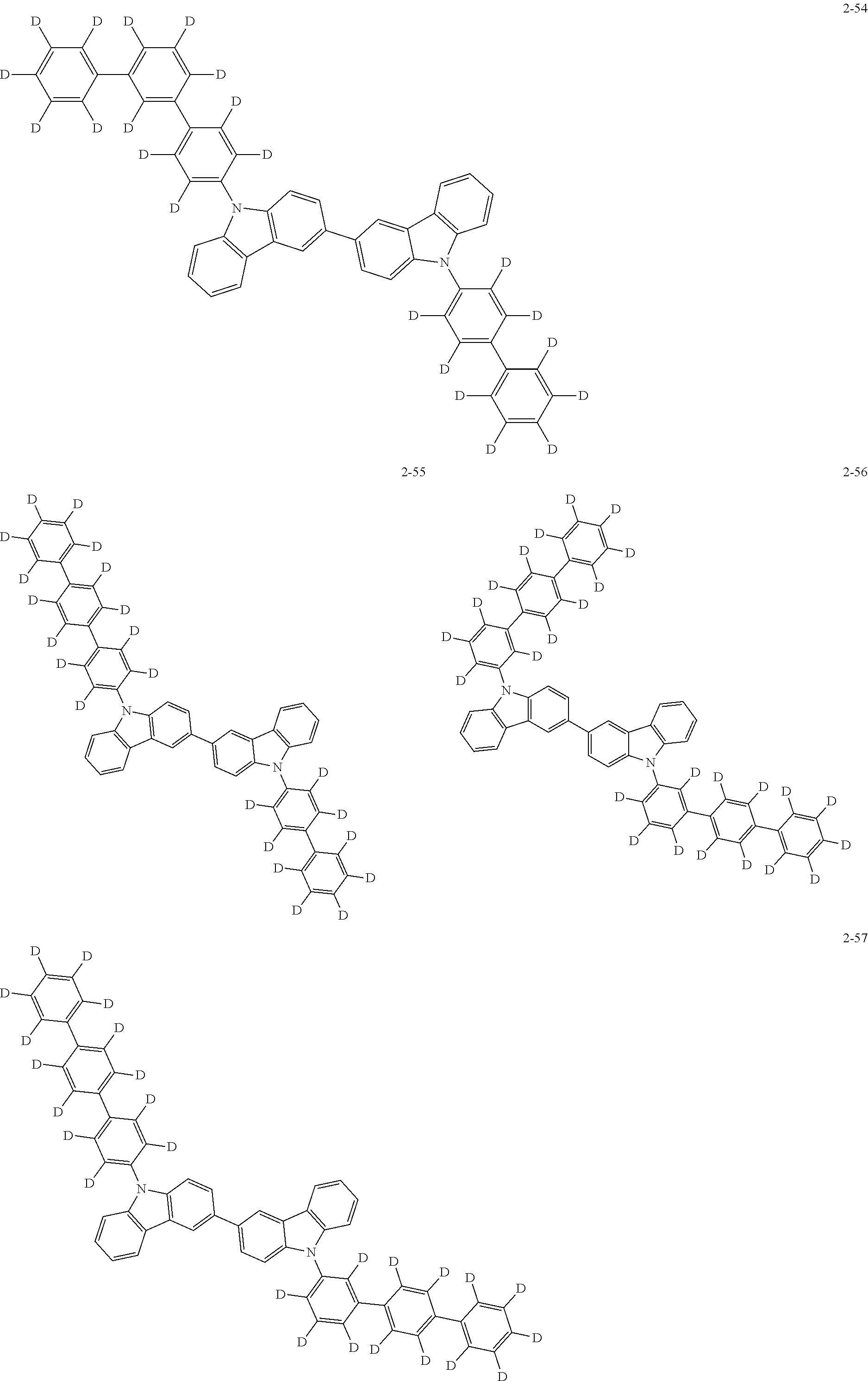

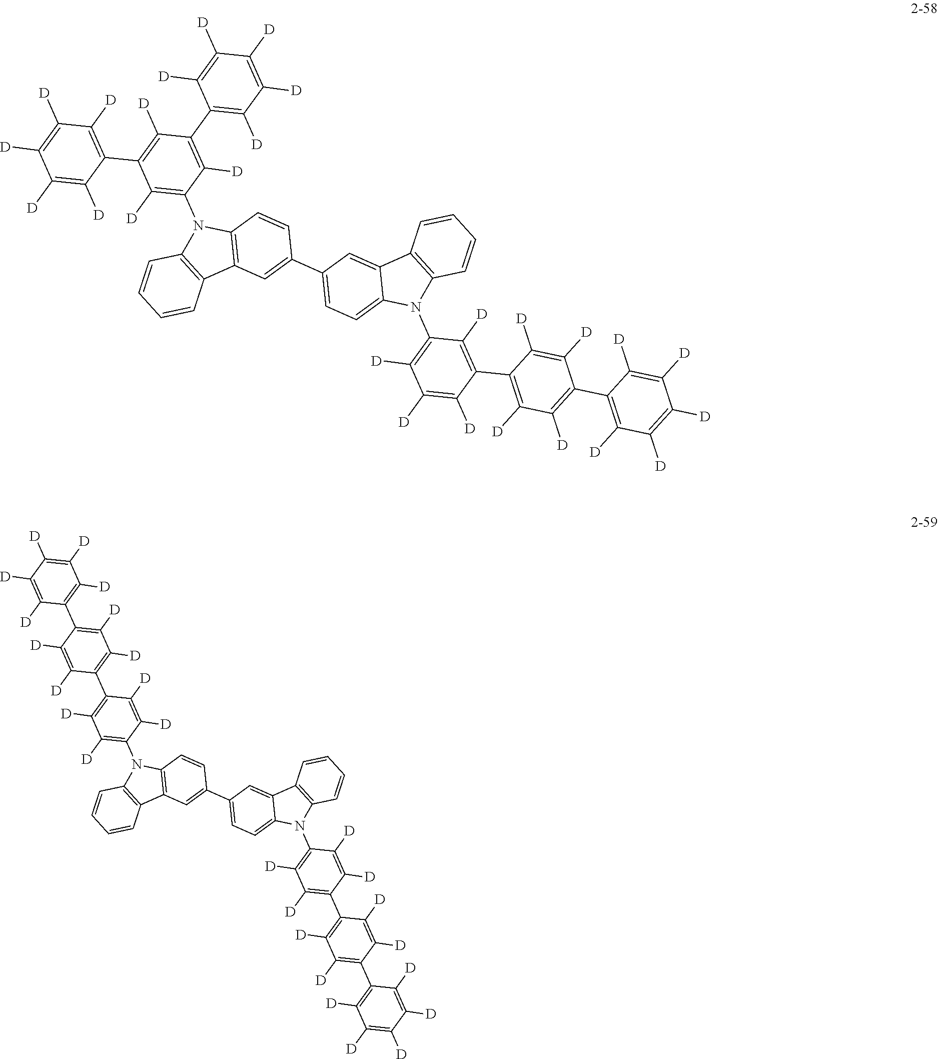

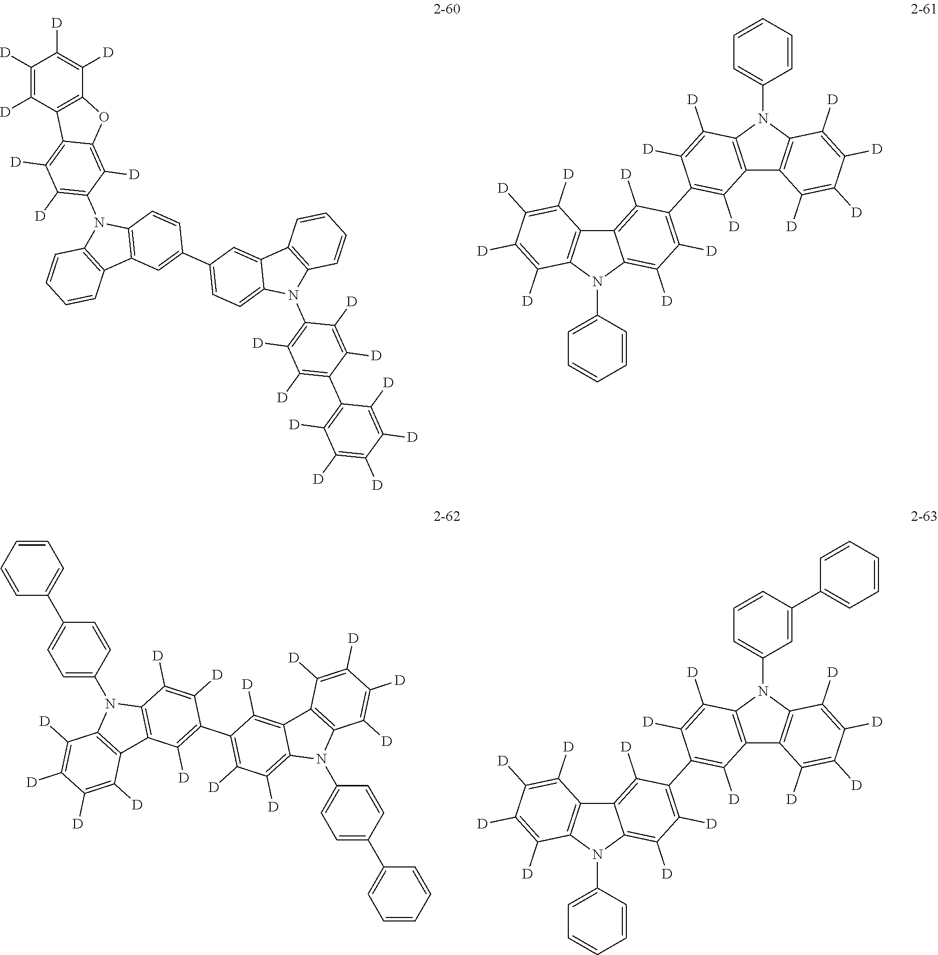

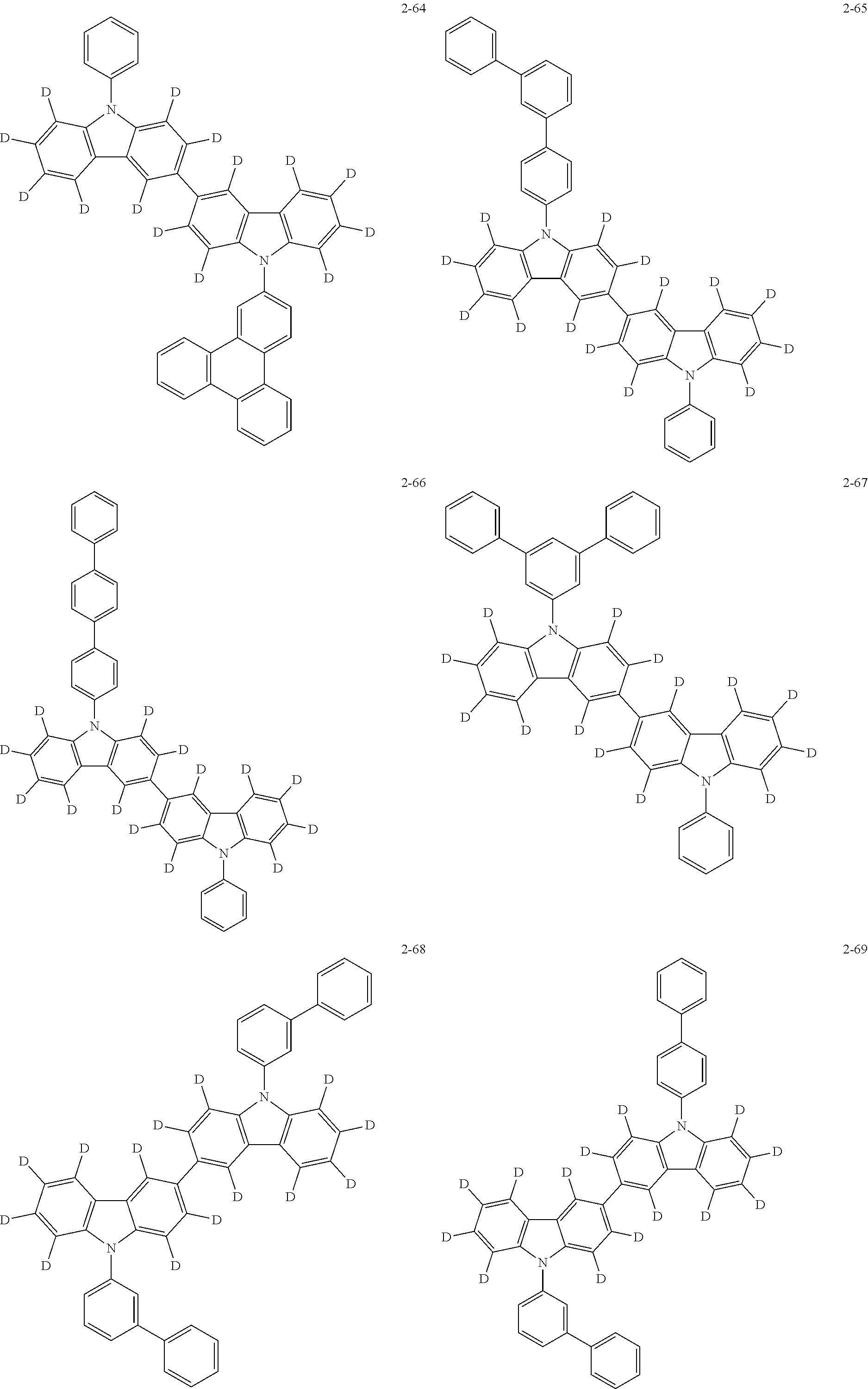

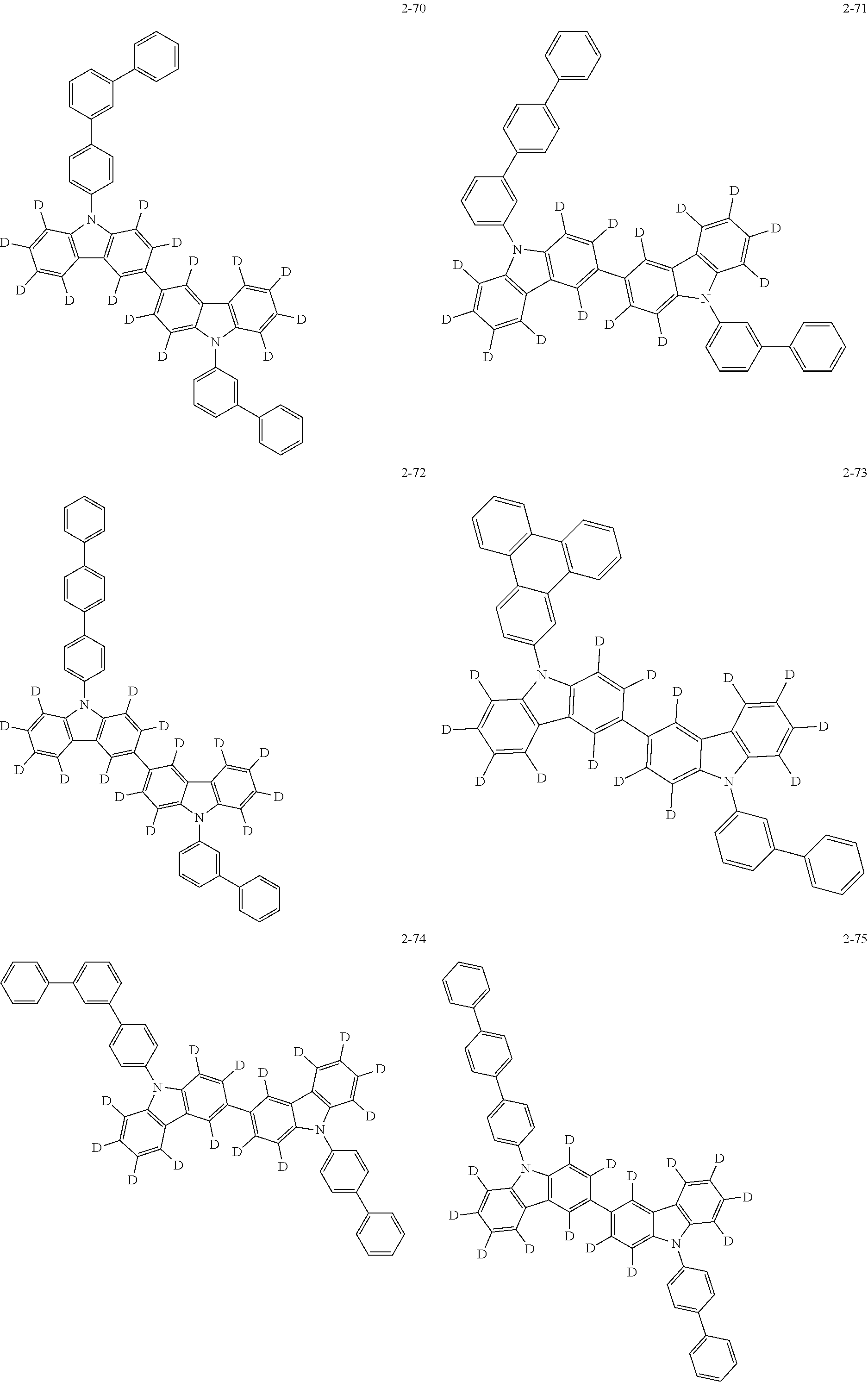

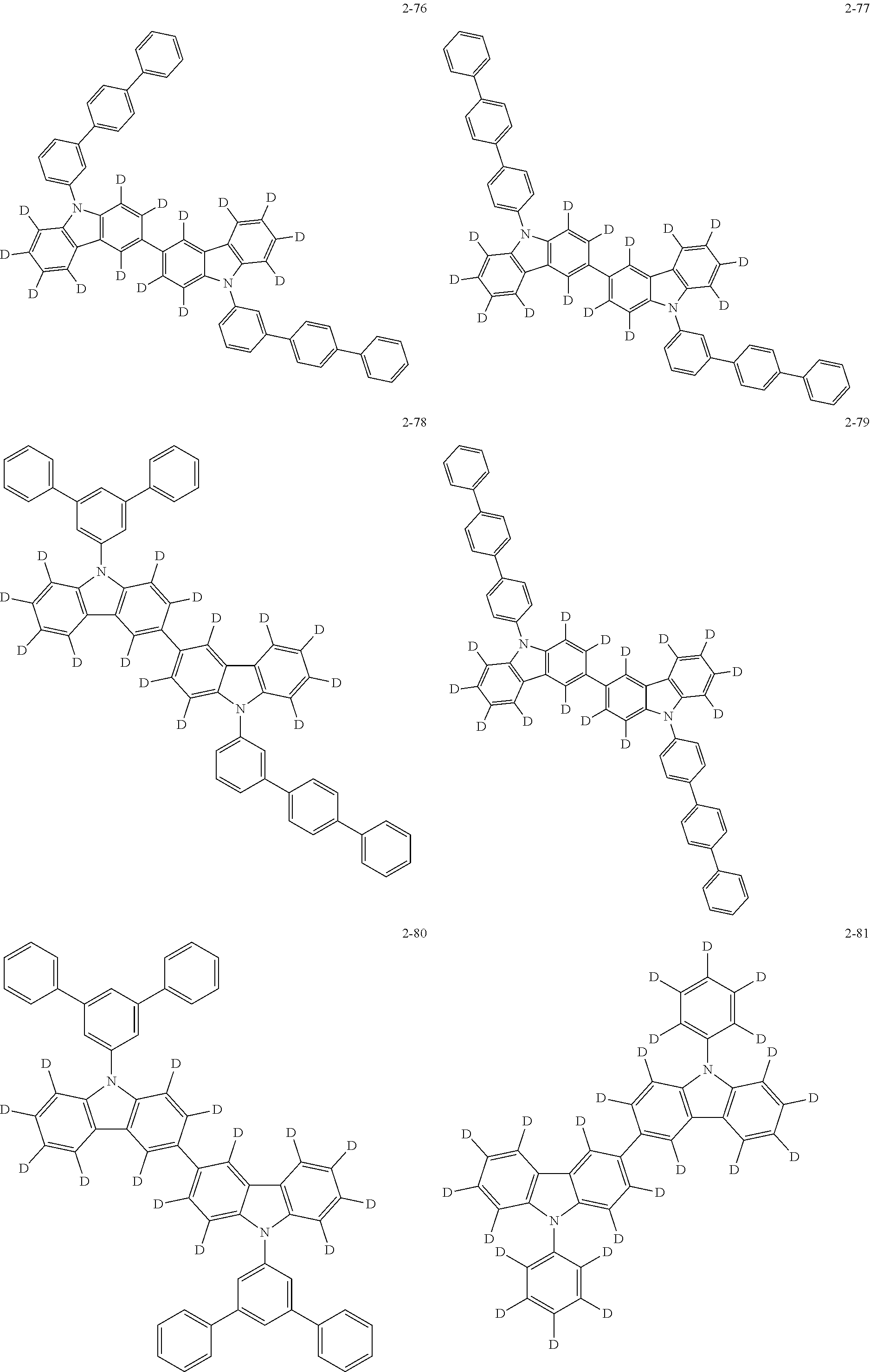

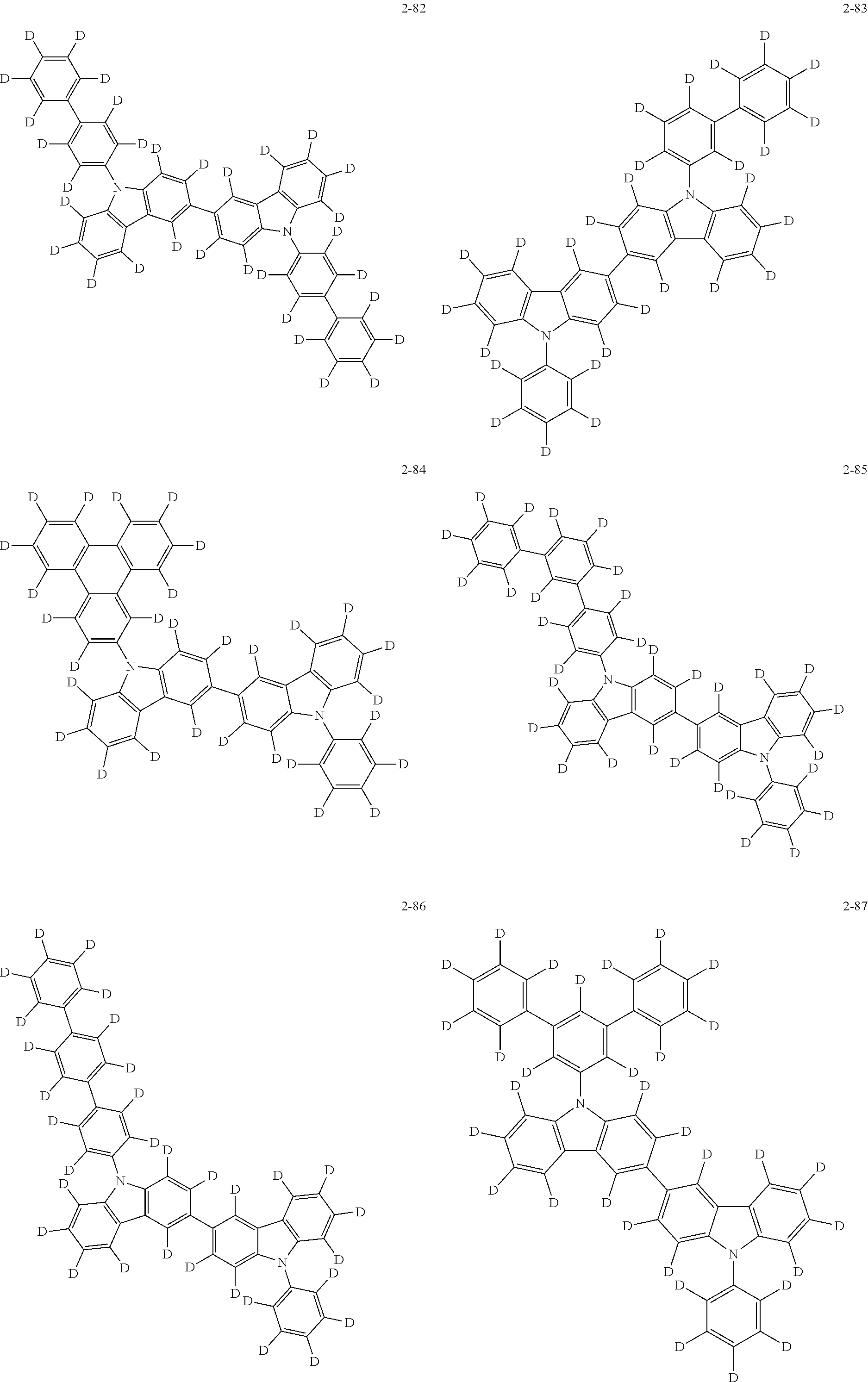

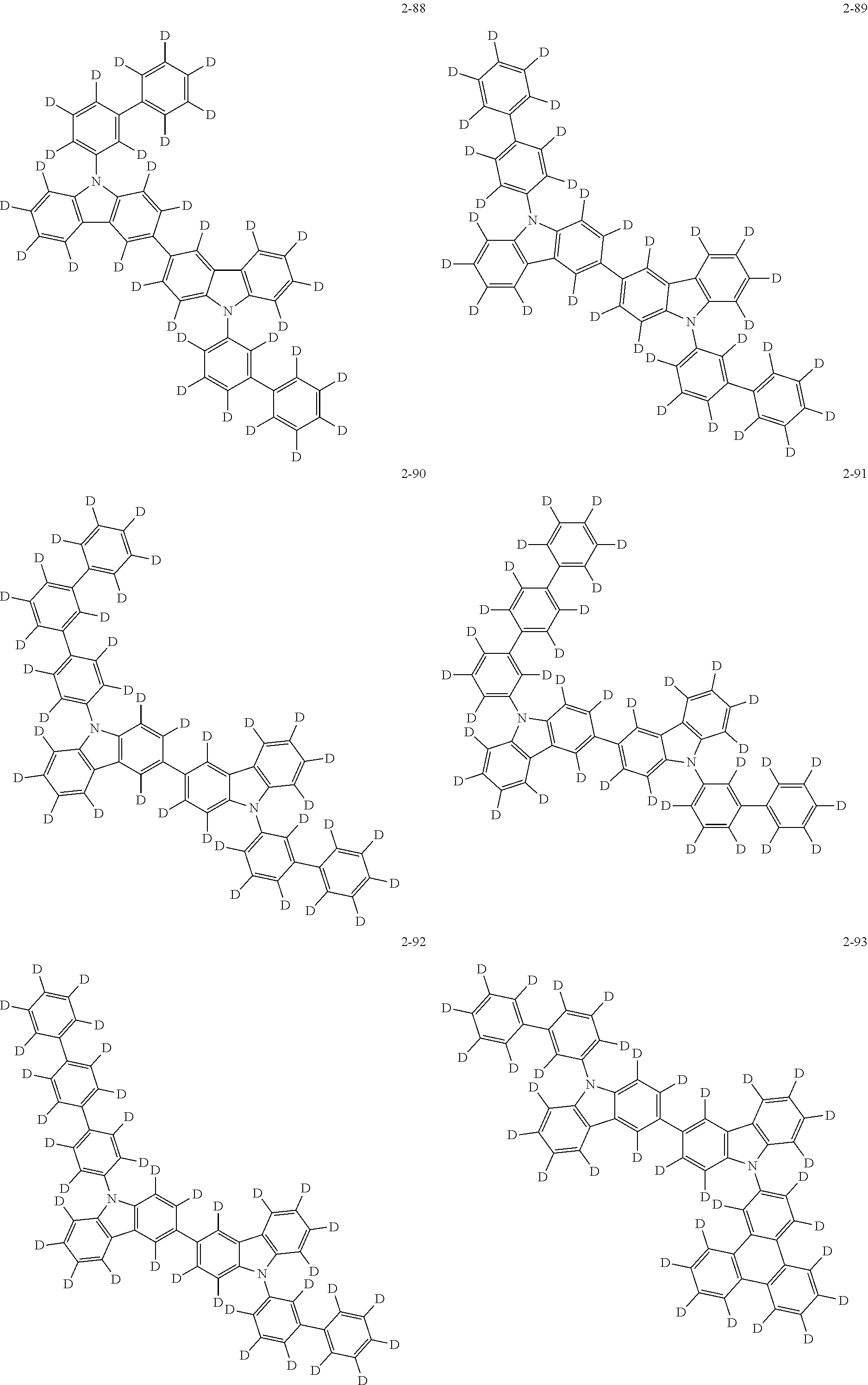

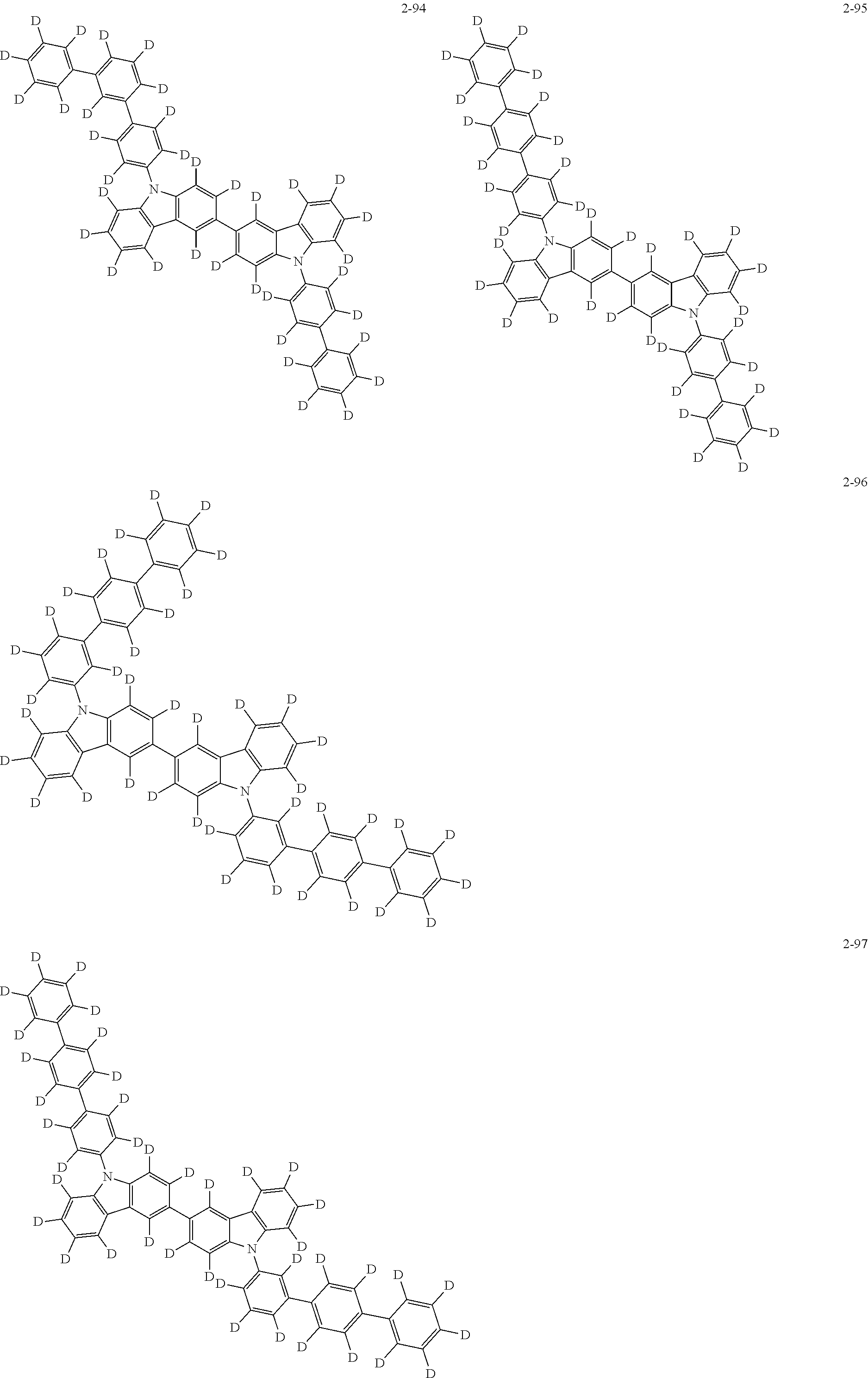

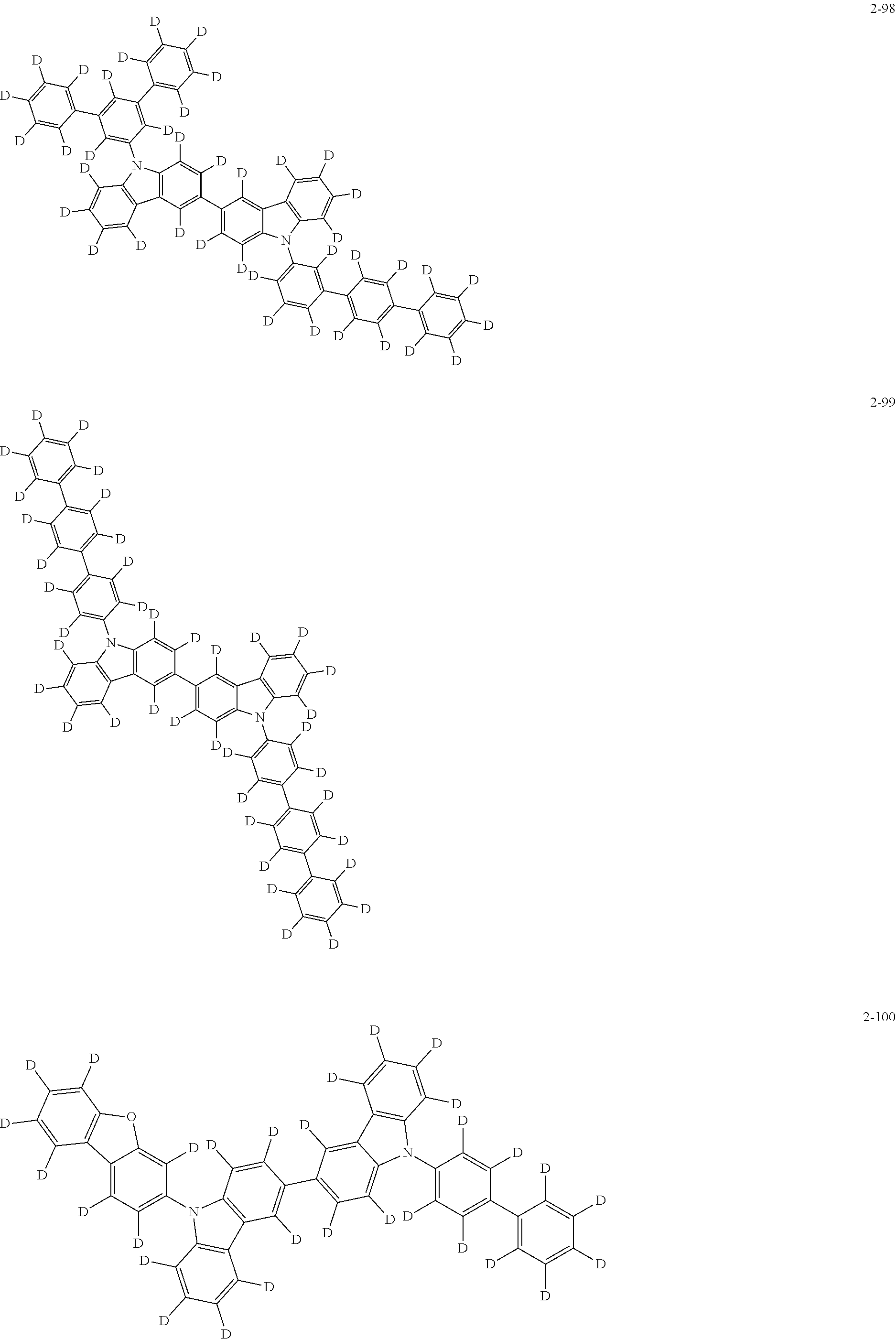

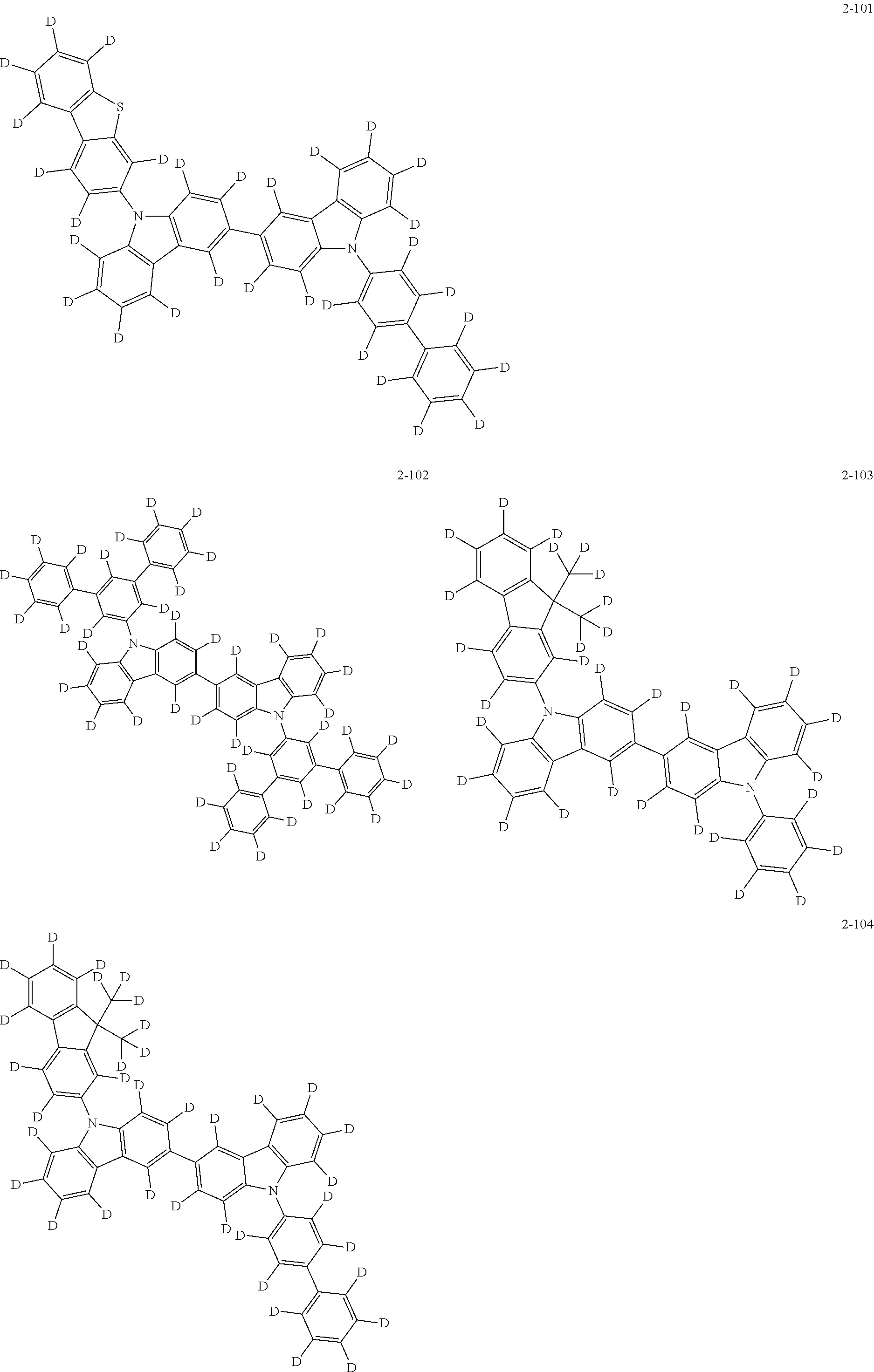

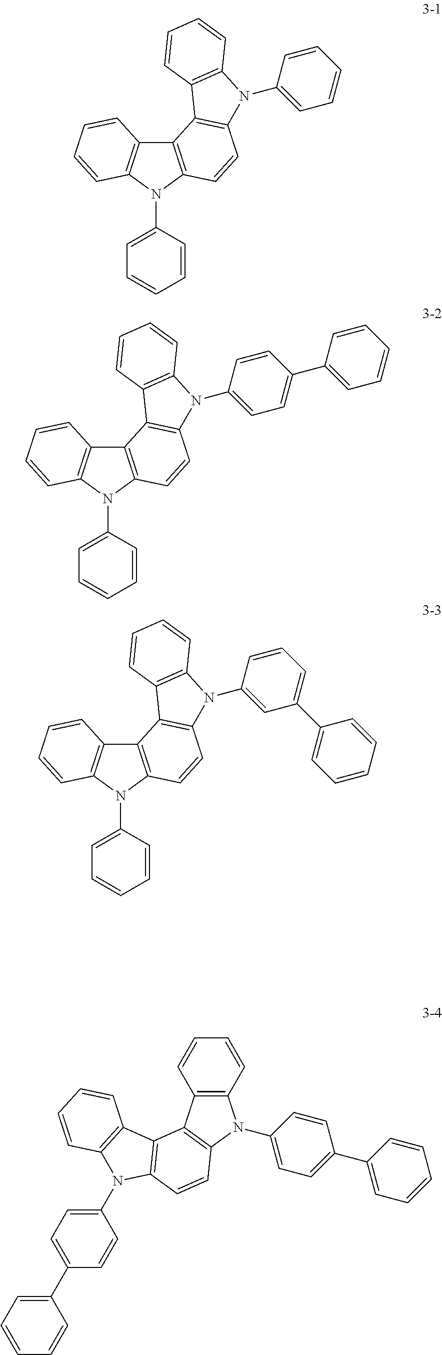

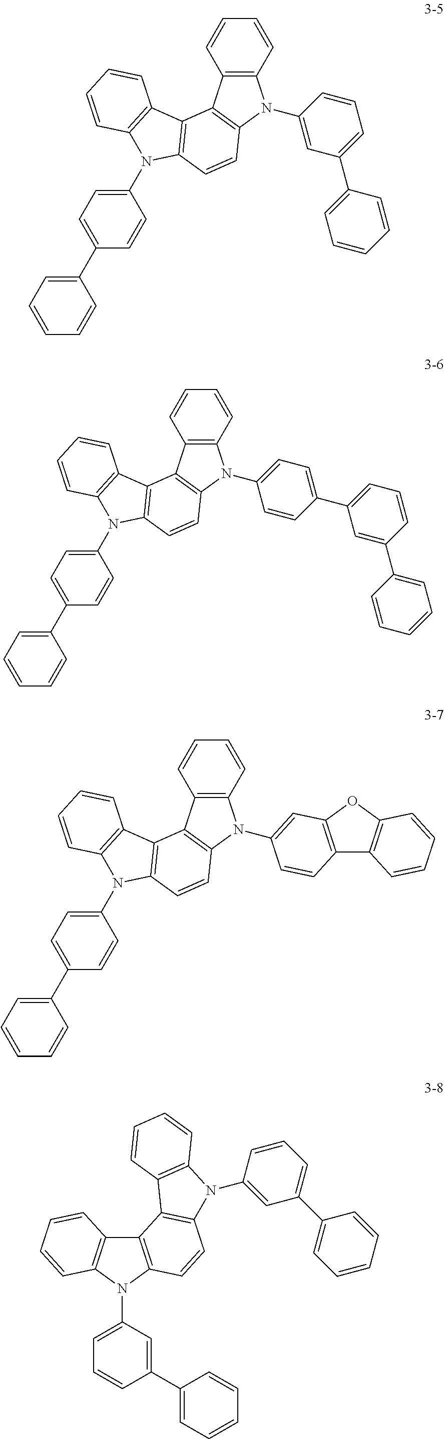 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6ccc(-c7cccc(-c8ccccc8)c7)cc6)c5ccc43)cc2)cc1 ; c1ccc(-c2ccc(-n3c4ccccc4c4c5c6ccccc6n(-c6ccc7c(c6)oc6ccccc67)c5ccc43)cc2)cc1 ; c1ccc(-c2cccc(-n3c4ccccc4c4c5c6ccccc6n(-c6cccc(-c7ccccc7)c6)c5ccc43)c2)cc1]

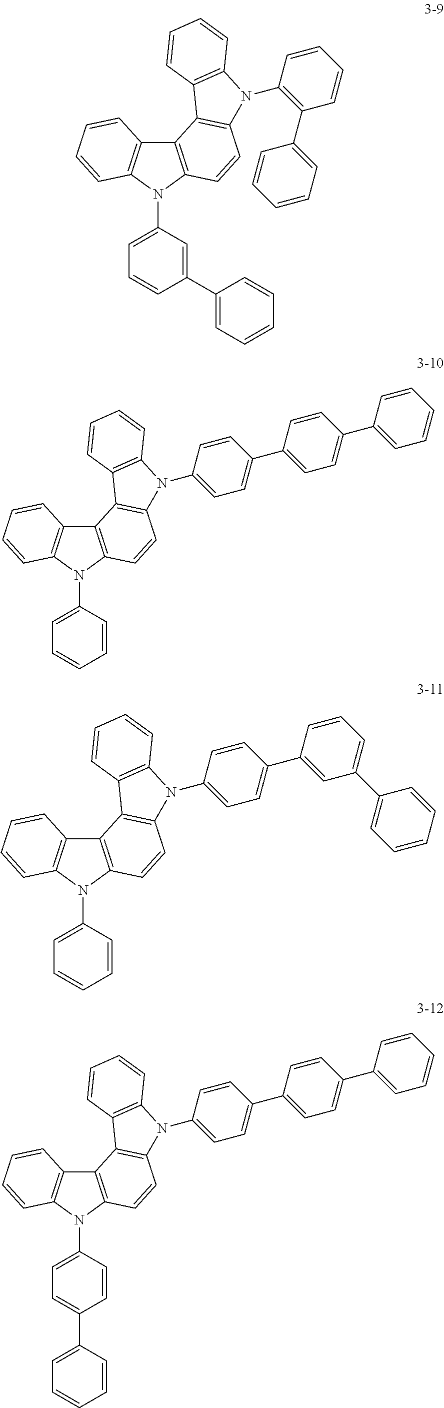 [SMILES: c1ccc(-c2cccc(-n3c4ccccc4c4c5c6ccccc6n(-c6ccccc6-c6ccccc6)c5ccc43)c2)cc1 ; c1ccc(-c2ccc(-c3ccc(-n4c5ccccc5c5c6c7ccccc7n(-c7ccccc7)c6ccc54)cc3)cc2)cc1 ; c1ccc(-c2cccc(-c3ccc(-n4c5ccccc5c5c6c7ccccc7n(-c7ccccc7)c6ccc54)cc3)c2)cc1 ; c1ccc(-c2ccc(-c3ccc(-n4c5ccccc5c5c6c7ccccc7n(-c7ccc(-c8ccccc8)cc7)c6ccc54)cc3)cc2)cc1]

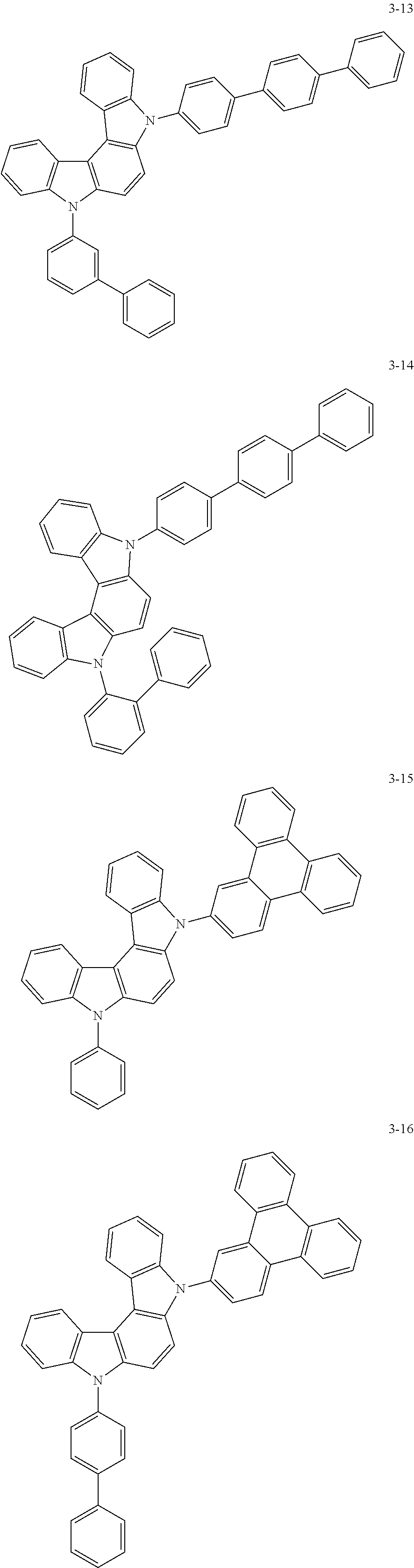 [SMILES: c1ccc(-c2ccc(-c3ccc(-n4c5ccccc5c5c6c7ccccc7n(-c7cccc(-c8ccccc8)c7)c6ccc54)cc3)cc2)cc1 ; c1ccc(-c2ccc(-c3ccc(-n4c5ccccc5c5c6c7ccccc7n(-c7ccccc7-c7ccccc7)c6ccc54)cc3)cc2)cc1 ; c1ccc(-n2c3ccccc3c3c4c5ccccc5n(-c5ccc6c7ccccc7c7ccccc7c6c5)c4ccc32)cc1 ; c1ccc(-c2ccc(-n3c4ccccc4c4c5c6ccccc6n(-c6ccc7c8ccccc8c8ccccc8c7c6)c5ccc43)cc2)cc1]

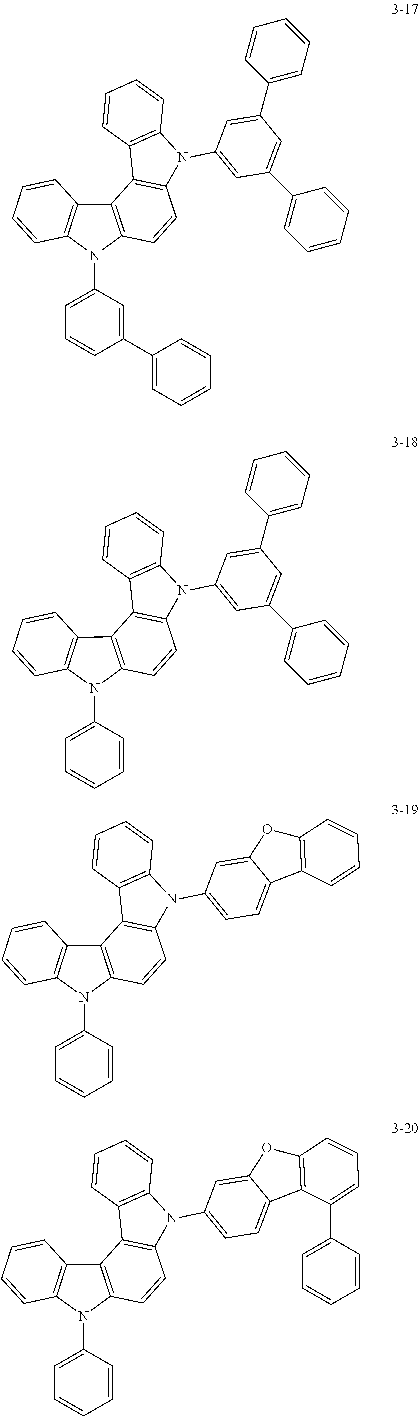

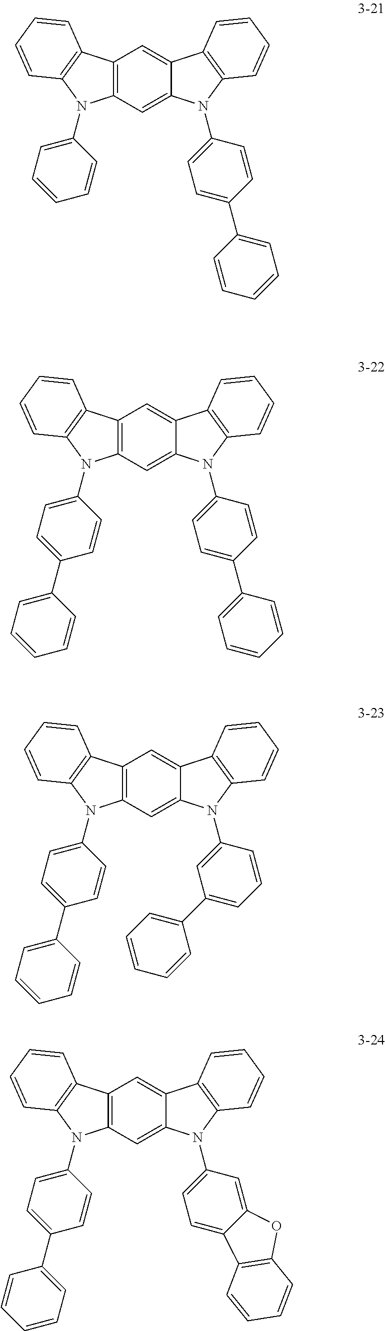 [SMILES: c1ccc(-c2ccc(-n3c4ccccc4c4cc5c6ccccc6n(-c6ccccc6)c5cc43)cc2)cc1 ; c1ccc(-c2ccc(-n3c4ccccc4c4cc5c6ccccc6n(-c6ccc(-c7ccccc7)cc6)c5cc43)cc2)cc1 ; c1ccc(-c2ccc(-n3c4ccccc4c4cc5c6ccccc6n(-c6cccc(-c7ccccc7)c6)c5cc43)cc2)cc1 ; c1ccc(-c2ccc(-n3c4ccccc4c4cc5c6ccccc6n(-c6ccc7c(c6)oc6ccccc67)c5cc43)cc2)cc1]

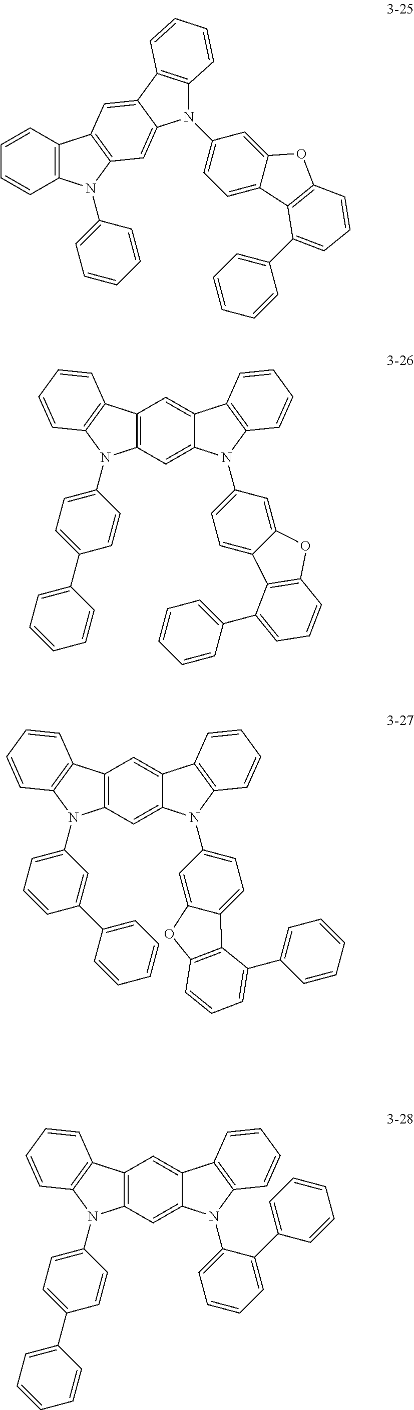 [SMILES: c1ccc(-c2cccc3oc4cc(-n5c6ccccc6c6cc7c8ccccc8n(-c8ccccc8)c7cc65)ccc4c23)cc1 ; c1ccc(-c2ccc(-n3c4ccccc4c4cc5c6ccccc6n(-c6ccc7c(c6)oc6cccc(-c8ccccc8)c67)c5cc43)cc2)cc1 ; c1ccc(-c2cccc(-n3c4ccccc4c4cc5c6ccccc6n(-c6ccc7c(c6)oc6cccc(-c8ccccc8)c67)c5cc43)c2)cc1 ; c1ccc(-c2ccc(-n3c4ccccc4c4cc5c6ccccc6n(-c6ccccc6-c6ccccc6)c5cc43)cc2)cc1]

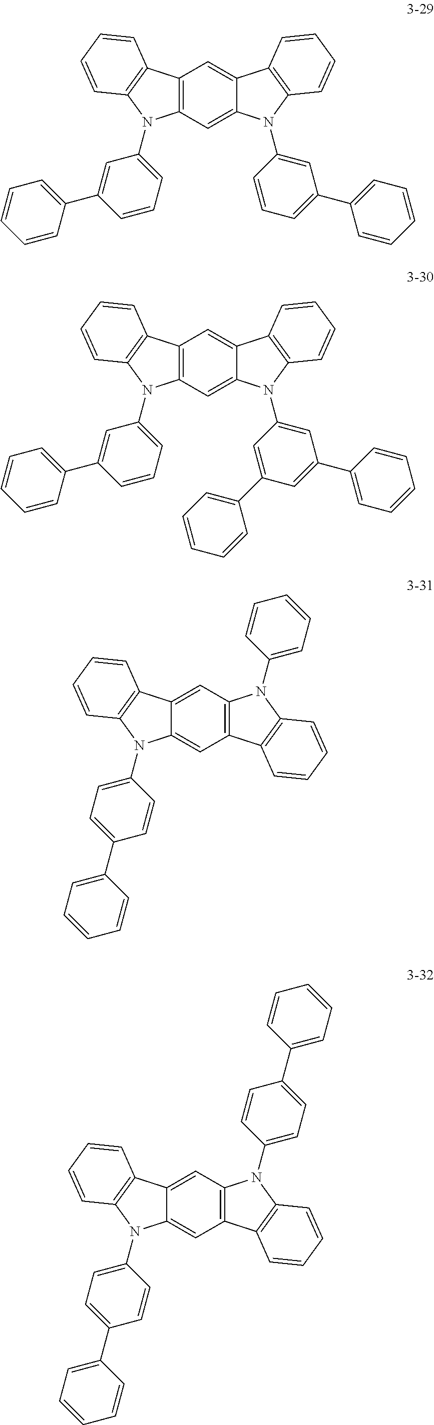 [SMILES: c1ccc(-c2cccc(-n3c4ccccc4c4cc5c6ccccc6n(-c6cccc(-c7ccccc7)c6)c5cc43)c2)cc1 ; c1ccc(-c2cccc(-n3c4ccccc4c4cc5c6ccccc6n(-c6cc(-c7ccccc7)cc(-c7ccccc7)c6)c5cc43)c2)cc1 ; c1ccc(-c2ccc(-n3c4ccccc4c4cc5c(cc43)c3ccccc3n5-c3ccccc3)cc2)cc1 ; c1ccc(-c2ccc(-n3c4ccccc4c4cc5c(cc43)c3ccccc3n5-c3ccc(-c4ccccc4)cc3)cc2)cc1]

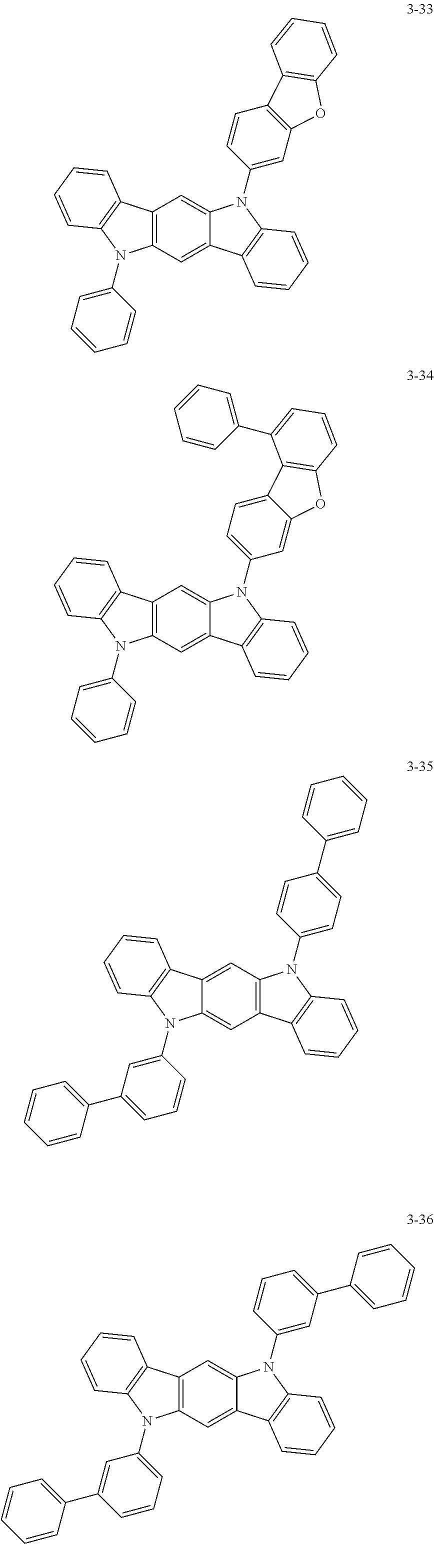 [SMILES: c1ccc(-n2c3ccccc3c3cc4c(cc32)c2ccccc2n4-c2ccc3c(c2)oc2ccccc23)cc1 ; c1ccc(-c2cccc3oc4cc(-n5c6ccccc6c6cc7c(cc65)c5ccccc5n7-c5ccccc5)ccc4c23)cc1 ; c1ccc(-c2ccc(-n3c4ccccc4c4cc5c(cc43)c3ccccc3n5-c3cccc(-c4ccccc4)c3)cc2)cc1 ; c1ccc(-c2cccc(-n3c4ccccc4c4cc5c(cc43)c3ccccc3n5-c3cccc(-c4ccccc4)c3)c2)cc1]

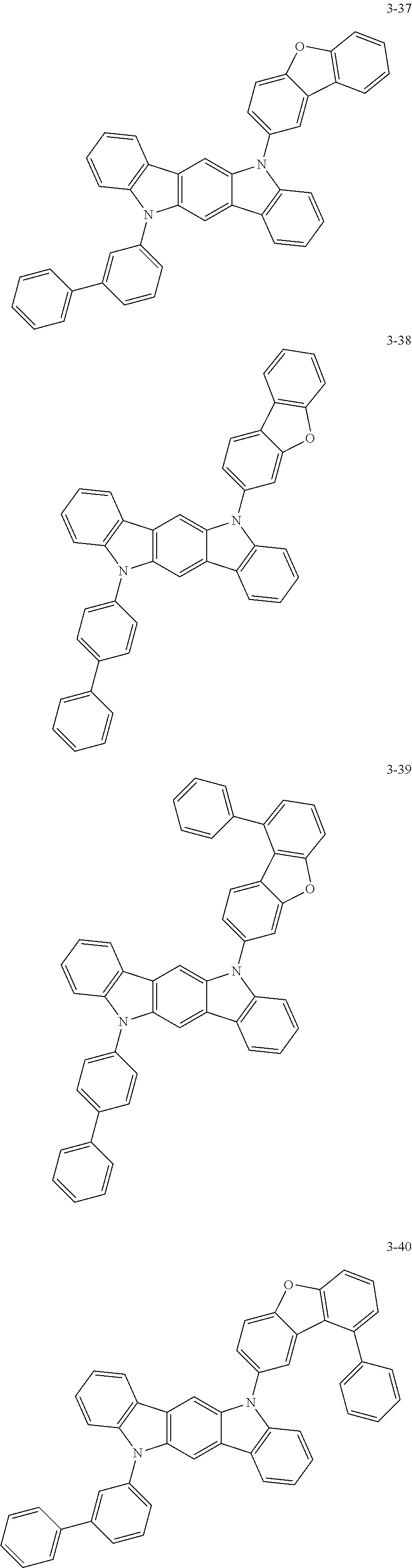 [SMILES: c1ccc(-c2cccc(-n3c4ccccc4c4cc5c(cc43)c3ccccc3n5-c3ccc4oc5ccccc5c4c3)c2)cc1 ; c1ccc(-c2ccc(-n3c4ccccc4c4cc5c(cc43)c3ccccc3n5-c3ccc4c(c3)oc3ccccc34)cc2)cc1 ; c1ccc(-c2ccc(-n3c4ccccc4c4cc5c(cc43)c3ccccc3n5-c3ccc4c(c3)oc3cccc(-c5ccccc5)c34)cc2)cc1 ; c1ccc(-c2cccc(-n3c4ccccc4c4cc5c(cc43)c3ccccc3n5-c3ccc4oc5cccc(-c6ccccc6)c5c4c3)c2)cc1]

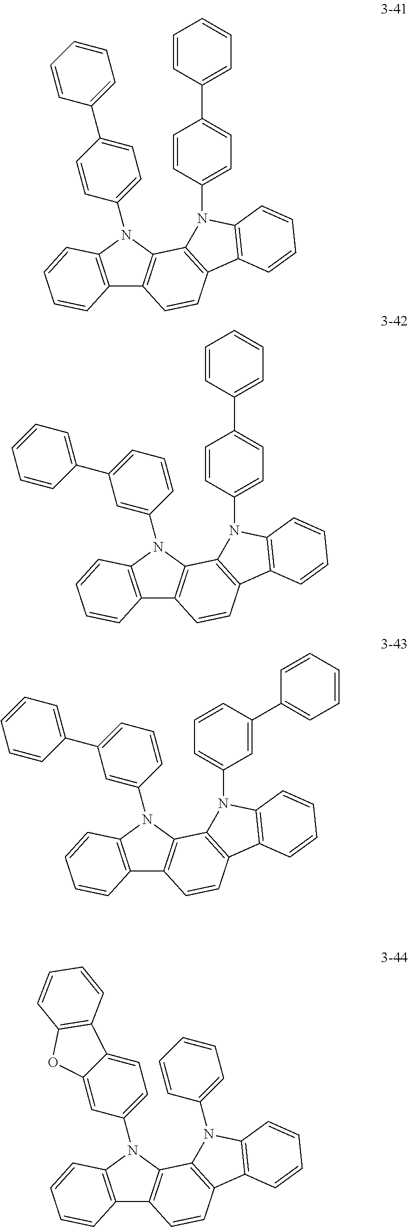

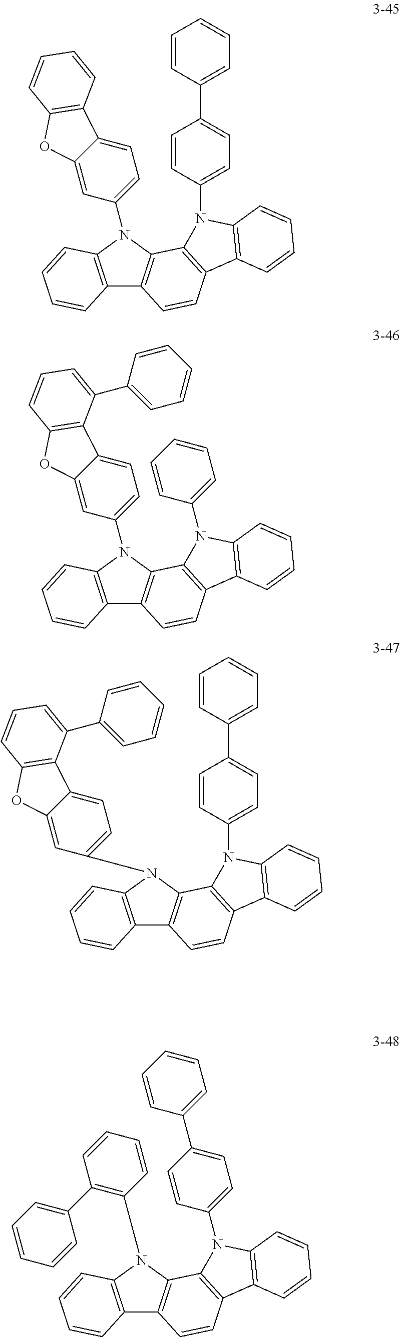

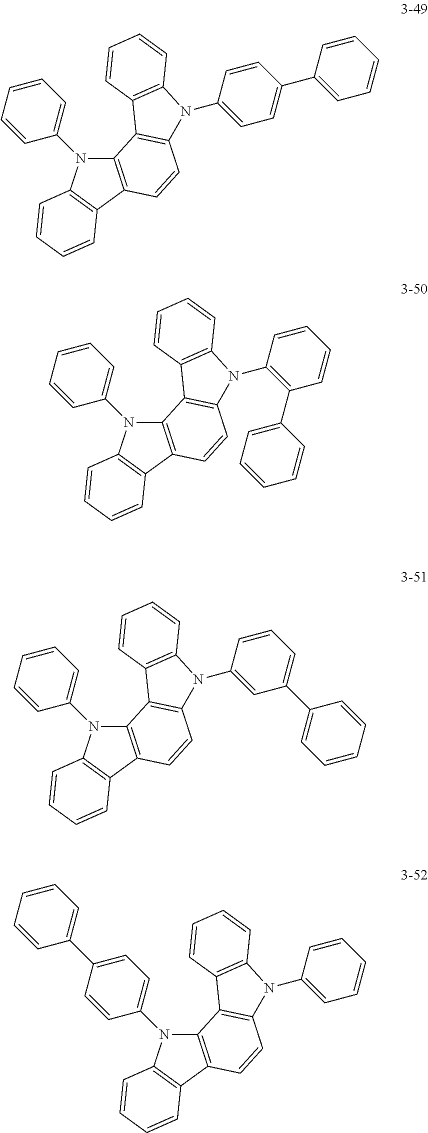 [SMILES: c1ccc(-c2ccc(-n3c4ccccc4c4c3ccc3c5ccccc5n(-c5ccccc5)c34)cc2)cc1 ; c1ccc(-c2ccccc2-n2c3ccccc3c3c2ccc2c4ccccc4n(-c4ccccc4)c23)cc1 ; c1ccc(-c2cccc(-n3c4ccccc4c4c3ccc3c5ccccc5n(-c5ccccc5)c34)c2)cc1 ; c1ccc(-c2ccc(-n3c4ccccc4c4ccc5c(c6ccccc6n5-c5ccccc5)c43)cc2)cc1]

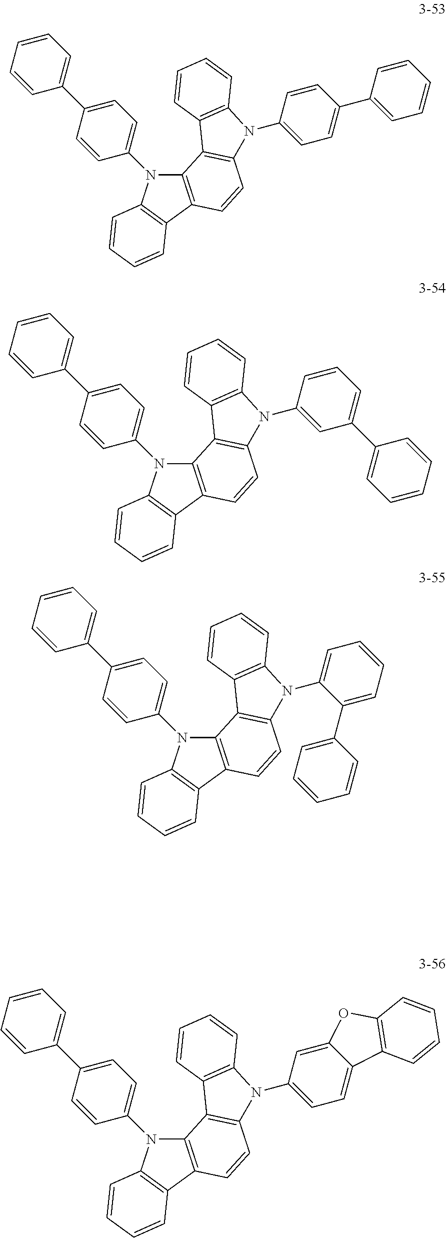 [SMILES: c1ccc(-c2ccc(-n3c4ccccc4c4c3ccc3c5ccccc5n(-c5ccc(-c6ccccc6)cc5)c34)cc2)cc1 ; c1ccc(-c2ccc(-n3c4ccccc4c4ccc5c(c6ccccc6n5-c5cccc(-c6ccccc6)c5)c43)cc2)cc1 ; c1ccc(-c2ccc(-n3c4ccccc4c4ccc5c(c6ccccc6n5-c5ccccc5-c5ccccc5)c43)cc2)cc1 ; c1ccc(-c2ccc(-n3c4ccccc4c4ccc5c(c6ccccc6n5-c5ccc6c(c5)oc5ccccc56)c43)cc2)cc1]

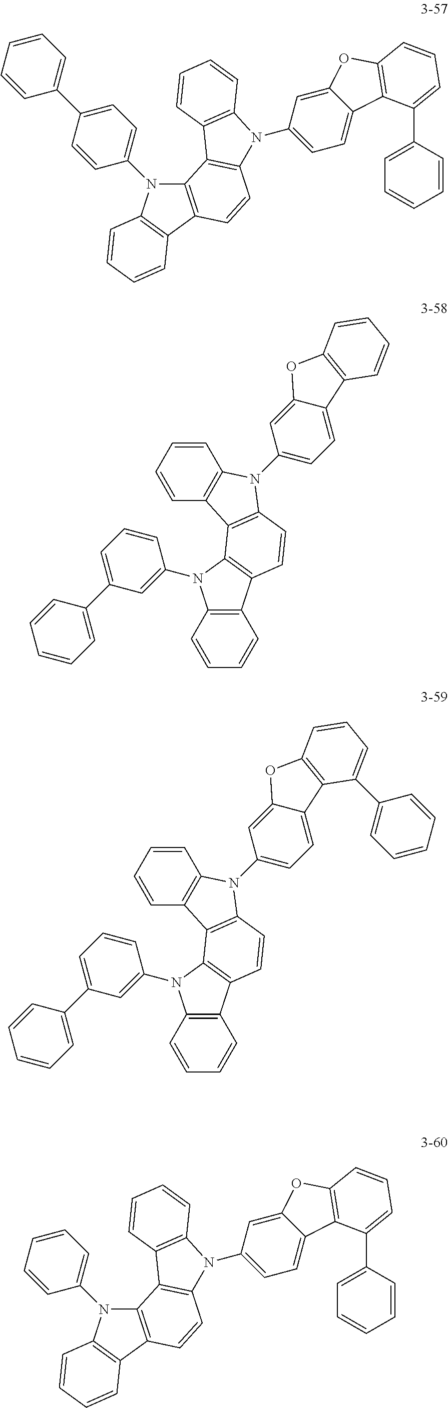 [SMILES: c1ccc(-c2ccc(-n3c4ccccc4c4ccc5c(c6ccccc6n5-c5ccc6c(c5)oc5cccc(-c7ccccc7)c56)c43)cc2)cc1 ; c1ccc(-c2cccc(-n3c4ccccc4c4ccc5c(c6ccccc6n5-c5ccc6c(c5)oc5ccccc56)c43)c2)cc1 ; c1ccc(-c2cccc(-n3c4ccccc4c4ccc5c(c6ccccc6n5-c5ccc6c(c5)oc5cccc(-c7ccccc7)c56)c43)c2)cc1 ; c1ccc(-c2cccc3oc4cc(-n5c6ccccc6c6c5ccc5c7ccccc7n(-c7ccccc7)c56)ccc4c23)cc1]

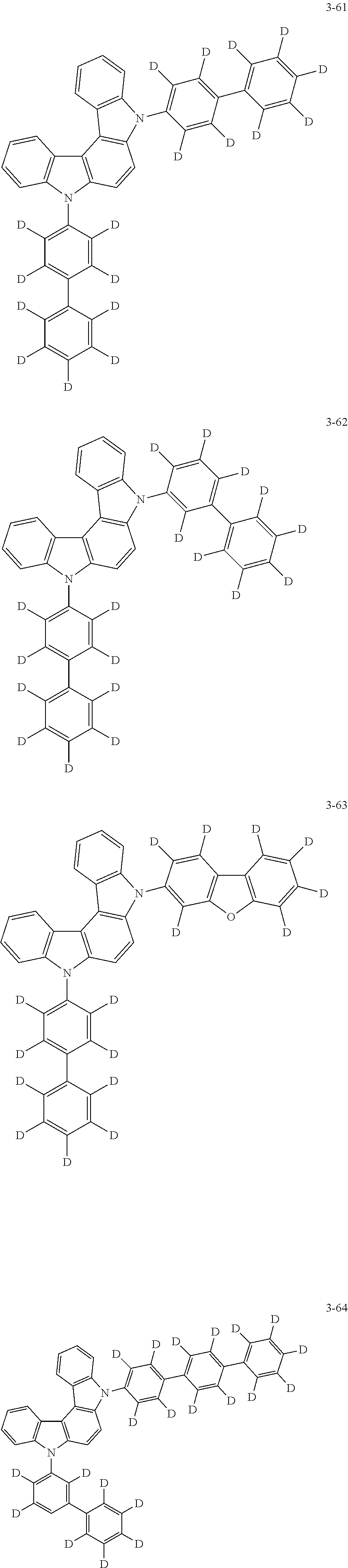

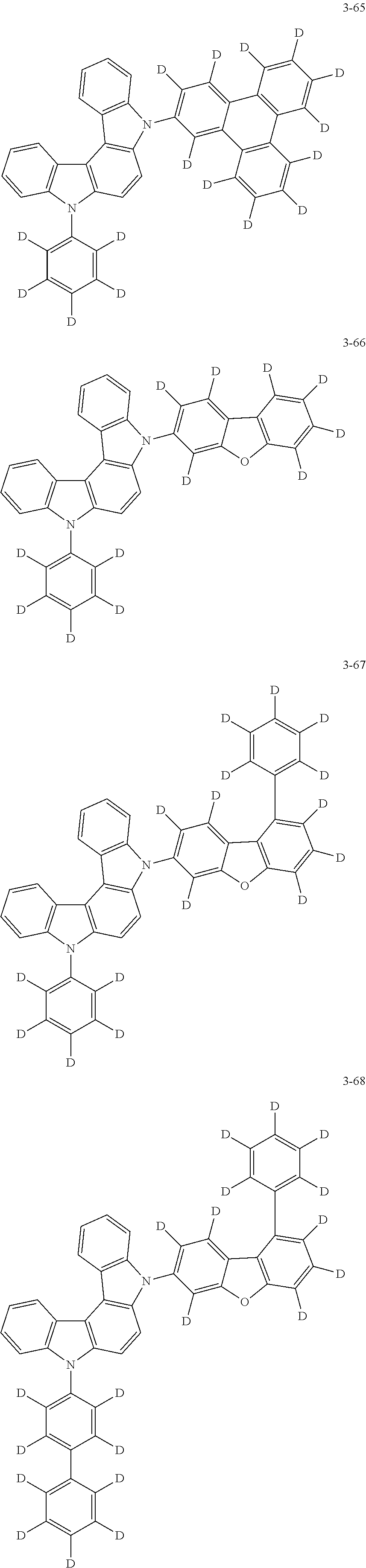

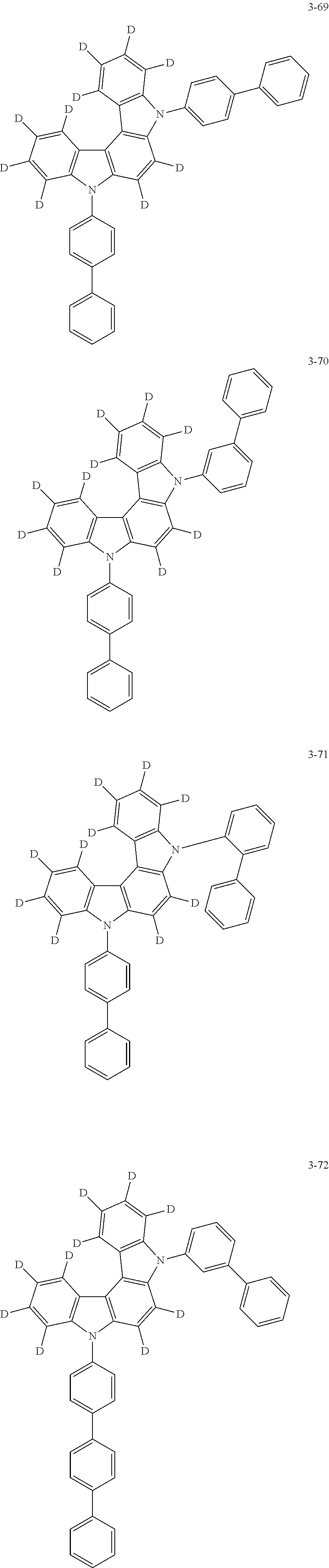

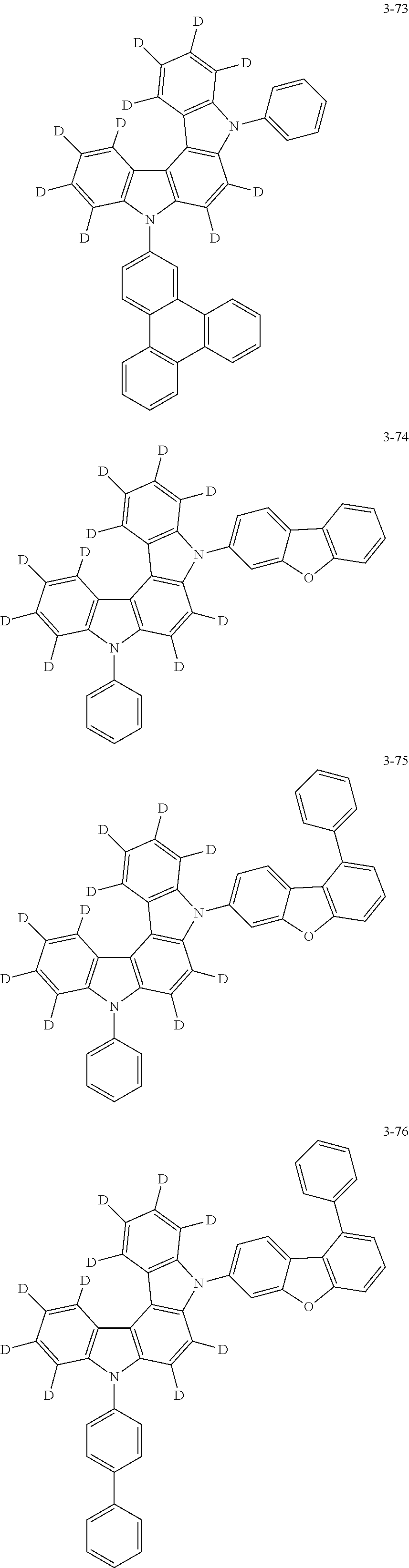

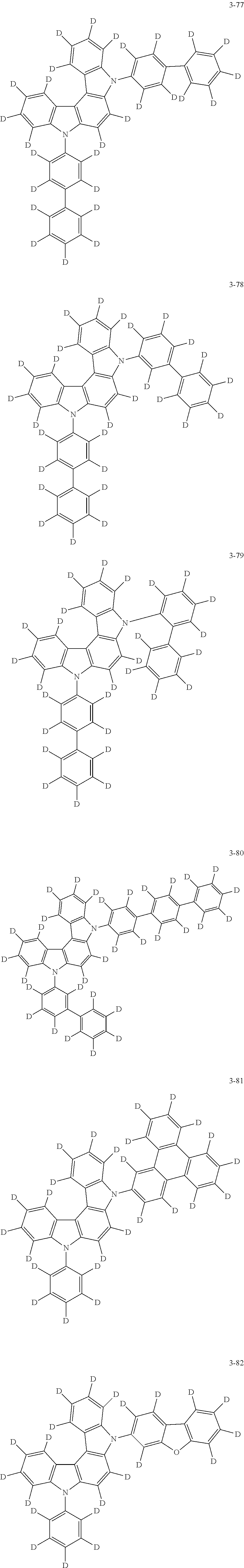

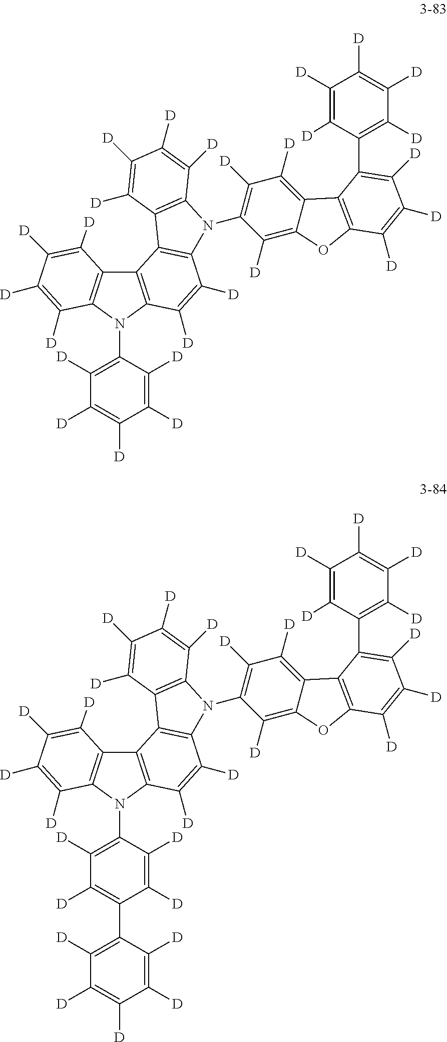 [SMILES: [2H]c1c([2H])c([2H])c(-c2c([2H])c([2H])c([2H])c3oc4c([2H])c(-n5c6c([2H])c([2H])c([2H])c([2H])c6c6c7c8c([2H])c([2H])c([2H])c([2H])c8n(-c8c([2H])c([2H])c([2H])c([2H])c8[2H])c7c([2H])c([2H])c65)c([2H])c([2H])c4c23)c([2H])c1[2H] ; [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c7c(oc8c([2H])c([2H])c([2H])c(-c9c([2H])c([2H])c([2H])c([2H])c9[2H])c87)c6[2H])c5c([2H])c([2H])c43)c([2H])c2[2H])c([2H])c1[2H]]

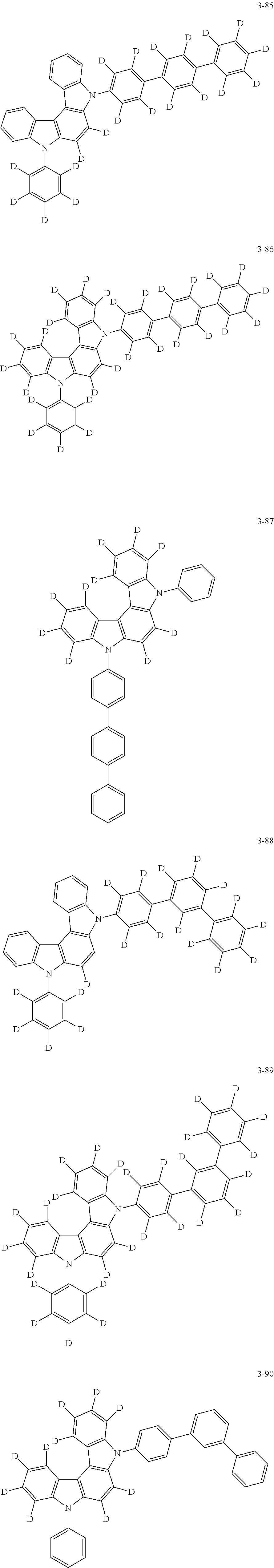

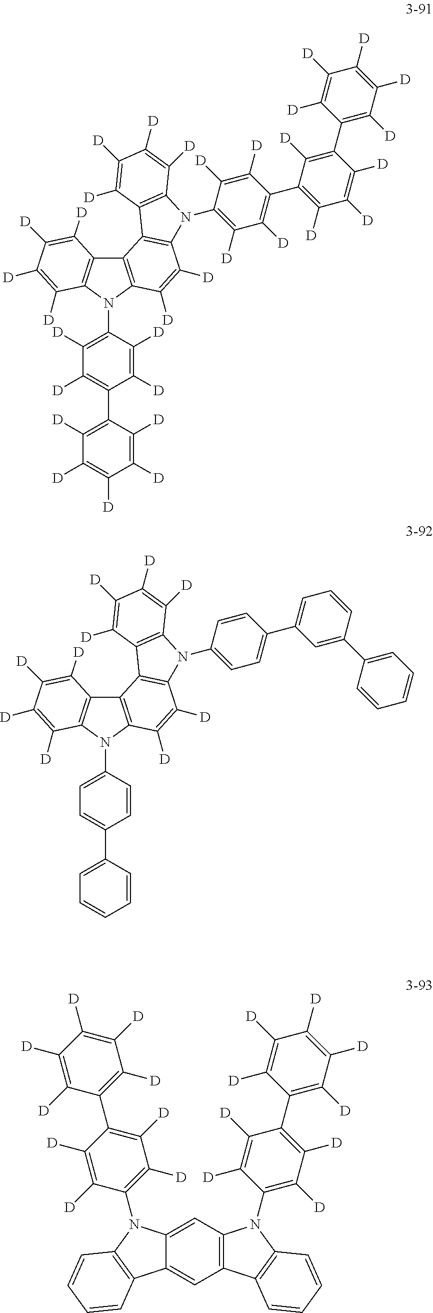

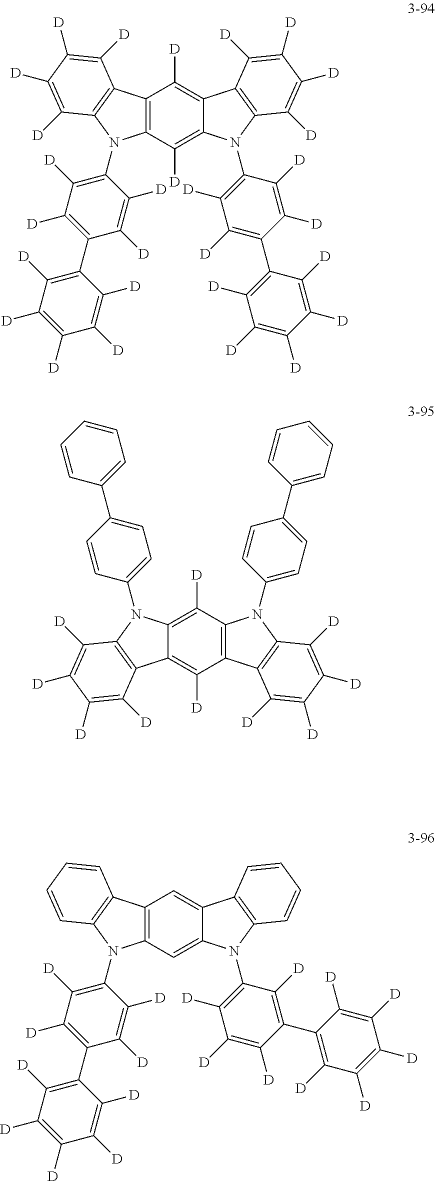

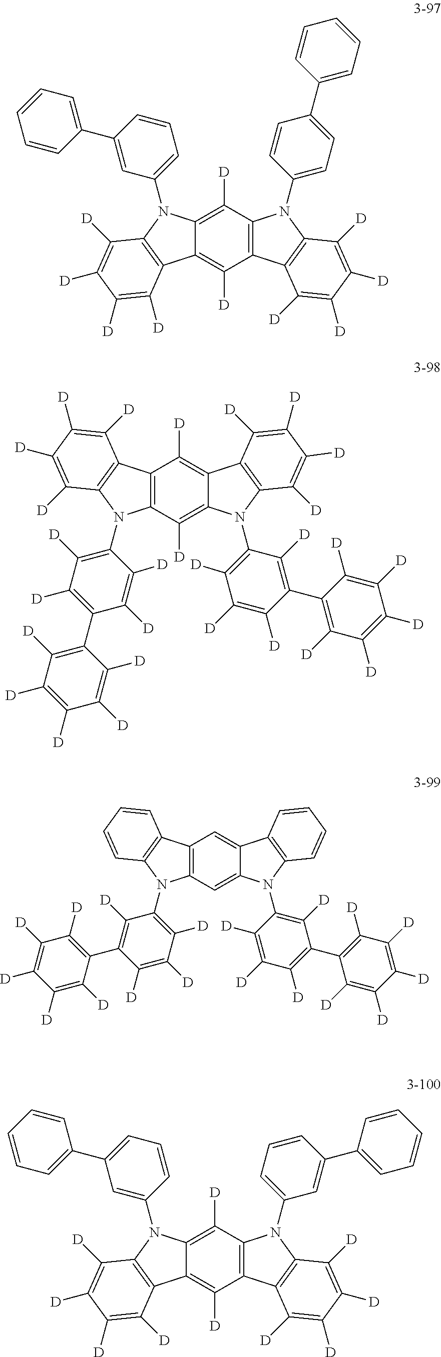

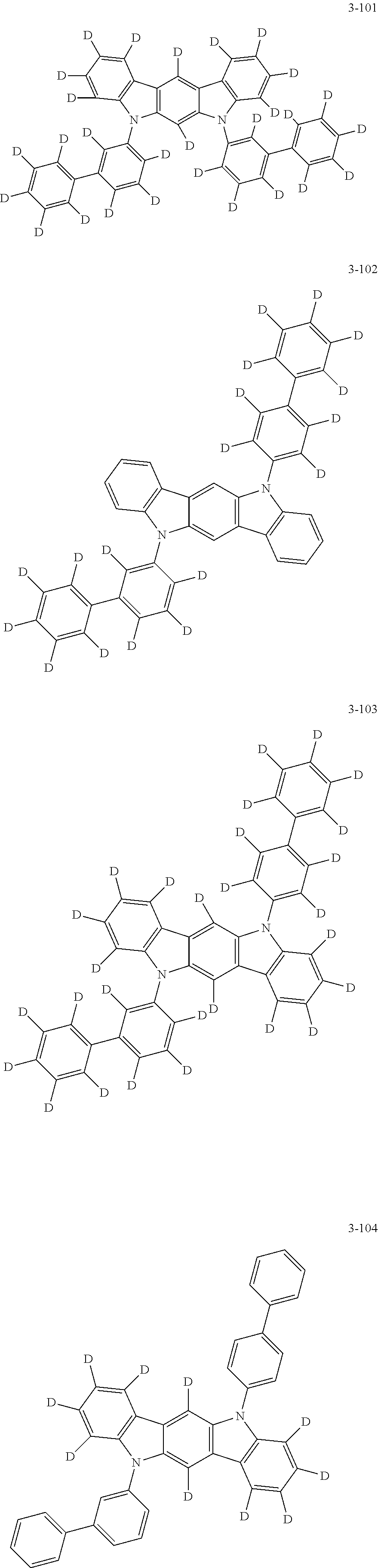

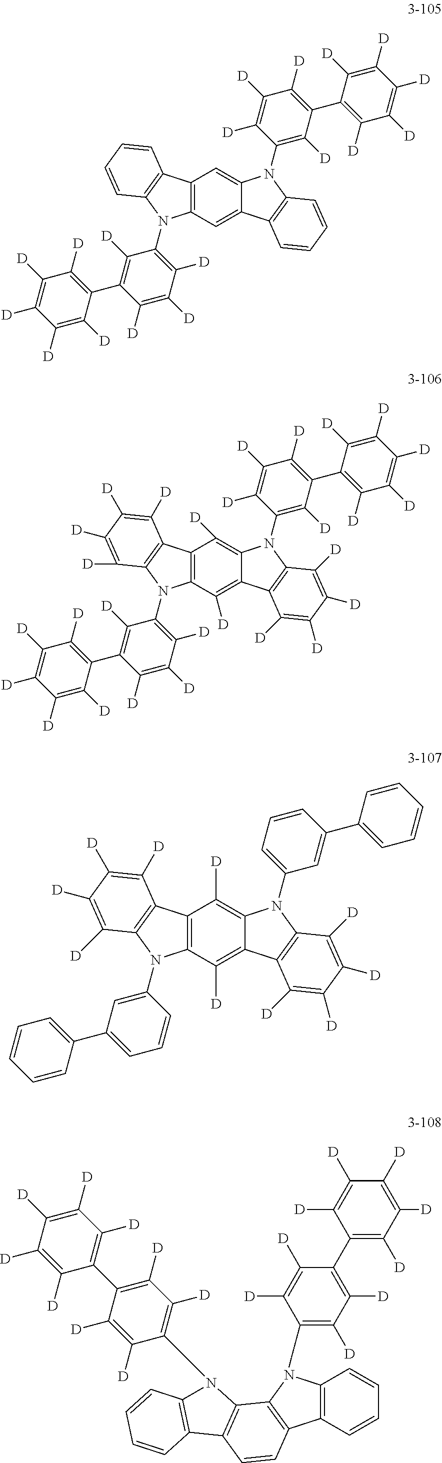

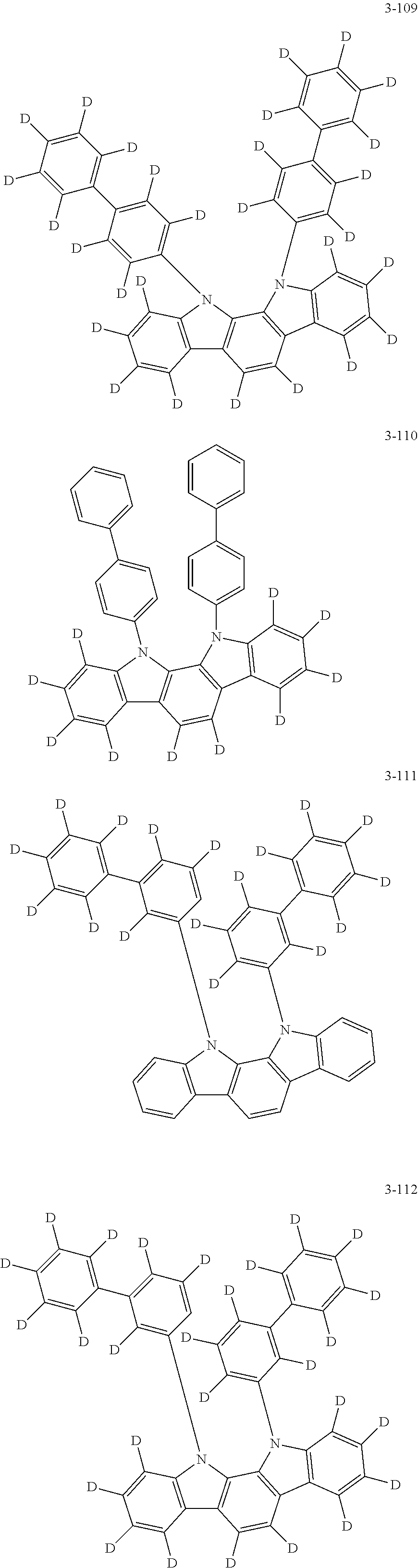

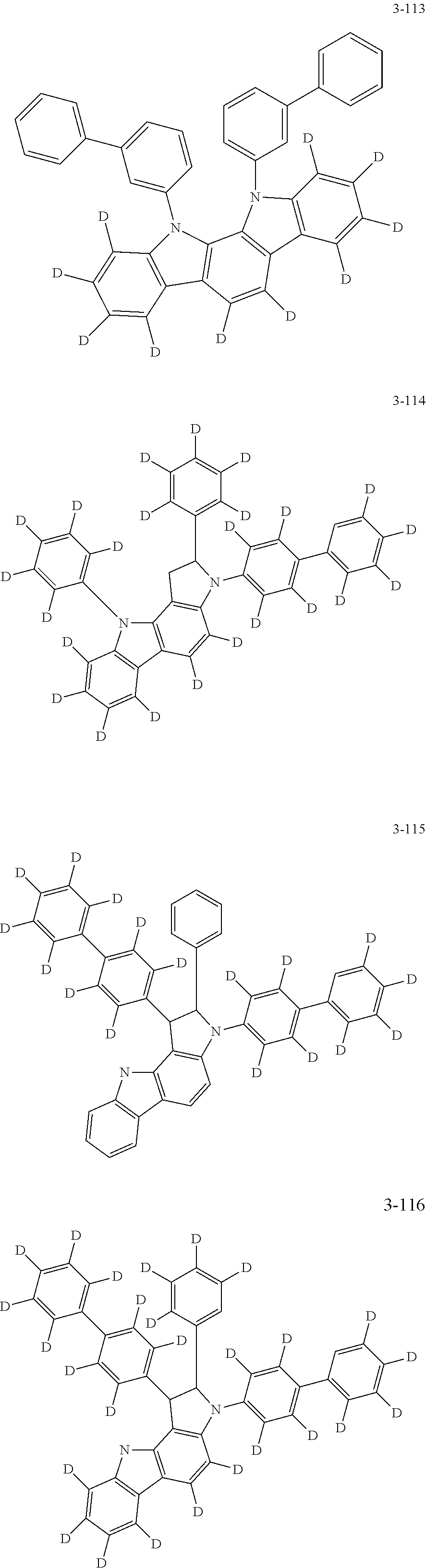

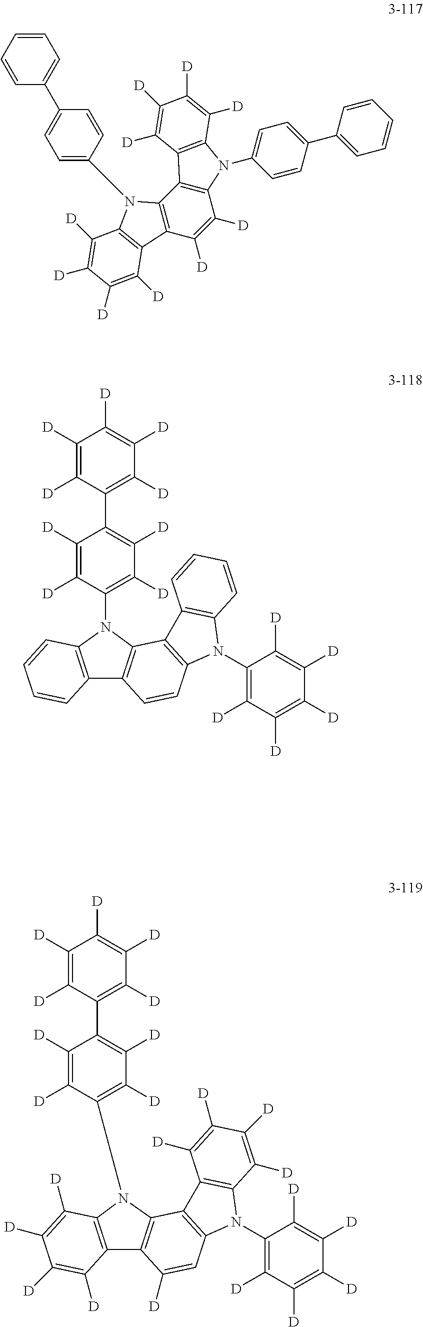 [SMILES: [2H]c1c([2H])c([2H])c2c(c1[2H])c1c([2H])c([2H])c3c(c4c([2H])c([2H])c([2H])c([2H])c4n3-c3ccc(-c4ccccc4)cc3)c1n2Cc1ccc(-c2ccccc2)cc1 ; [2H]c1c([2H])c([2H])c(-c2c([2H])c([2H])c(-n3c4ccccc4c4ccc5c(c6ccccc6n5-c5c([2H])c([2H])c([2H])c([2H])c5[2H])c43)c([2H])c2[2H])c([2H])c1[2H] ; [2H]c1c([2H])c([2H])c(-c2c([2H])c([2H])c(CCn3c4c([2H])c([2H])c([2H])c([2H])c4c4c([2H])cc5c(c6c([2H])c([2H])c([2H])c([2H])c6n5-c5c([2H])c([2H])c([2H])c([2H])c5[2H])c43)c([2H])c2[2H])c([2H])c1[2H]]

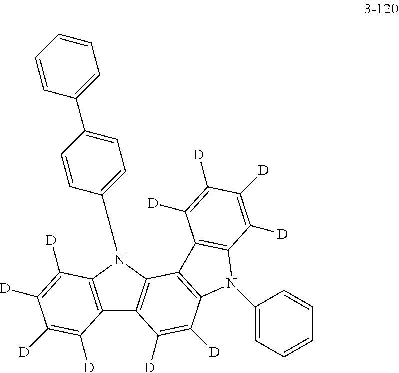 [SMILES: [2H]c1c([2H])c([2H])c2c(c1[2H])c1c([2H])c([2H])c3c(c4c([2H])c([2H])c([2H])c([2H])c4n3-c3ccccc3)c1n2Cc1ccc(-c2ccccc2)cc1]

Furthermore, another exemplary embodiment of the present application provides a composition for an organic material layer of an organic light emitting device, which includes the heterocyclic compound represented by Chemical Formula 1, and the heterocyclic compound represented by Chemical Formula 2 or 3.

The specific contents on the heterocyclic compound represented by Chemical Formula 1 and the heterocyclic compound represented by Chemical Formula 2 or 3 are the same as those described above.

The weight ratio of the heterocyclic compound represented by Chemical Formula 1:the heterocyclic compound represented by Chemical Formula 2 or 3 in the composition may be 1:10 to 10:1, 1:8 to 8:1, 1:5 to 5:1, and 1:2 to 2:1, but is not limited thereto.

The composition may be used when an organic material for an organic light emitting device is formed, and particularly, may be more preferably used when a host of a light emitting layer is formed.

In an exemplary embodiment of the present application, the organic material layer includes the heterocyclic compound represented by Chemical Formula 1, and the heterocyclic compound represented by Chemical Formula 2 or 3, and may be used together with a phosphorescent dopant.

In an exemplary embodiment of the present application, the organic material layer includes the heterocyclic compound represented by Chemical Formula 1, and the heterocyclic compound represented by Chemical Formula 2 or 3, and may be used together with an iridium-based dopant.

As a material for the phosphorescent dopant, those known in the art may be used.

For example, phosphorescent dopant materials represented by LL′MX′, LL′L″M, LMX′X″, L2MX′ and L3M may be used, but the scope of the present invention is not limited by these examples.

Here, L, L′, L″, X′ and X″ are bidentate ligands different from each other, and M is a metal forming an octahedral complex.

M may be iridium, platinum, osmium, and the like.

L is an anionic, bidendate ligand coordinated on M by the iridium-based dopant by sp2 carbon and a heteroatom, and X may perform a function of trapping electrons or holes. Non-limiting examples of L include 2-(1-naphthyl)benzoxazole, (2-phenylbenzoxazole), (2-phenylbenzothiazole), (2-phenylbenzothiazole), (7,8-benzoquinoline), (thiophene group pyrizine), phenylpyridine, benzothiophene group pyrizine, 3-methoxy-2-phenylpyridine, thiophene group pyrizine, tolylpyridine, and the like. Non-limiting examples of X′ and X″ include acetylacetonate (acac), hexafluoroacetylacetonate, salicylidene, picolinate, 8-hydroxyquinolinate, and the like.

More specific examples thereof will be shown below, but the present application is not limited only to these examples.

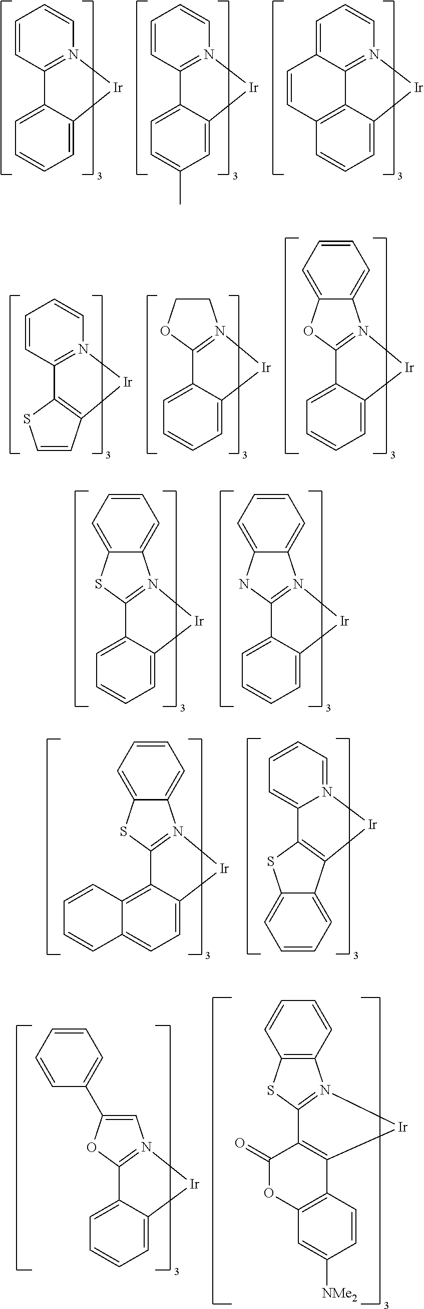

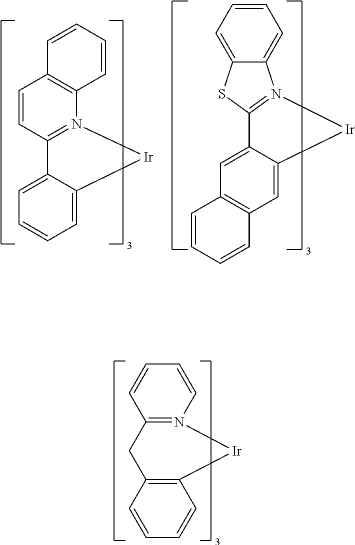

In an exemplary embodiment of the present application, as the iridium-based dopant, Ir(ppy)₃ as a green phosphorescent dopant may be used.

In an exemplary embodiment of the present application, the content of the dopant may be 1% to 15%, preferably 3% to 10%, and more preferably 5% to 10% based on the entire light emitting layer.

In the organic light emitting device of the present invention, the organic material layer includes an electron injection layer or an electron transport layer, and the electron injection layer or electron transport layer may include the heterocyclic compound.

In another organic light emitting device, the organic material layer includes an electron blocking layer or a hole blocking layer, and the electron blocking layer or the hole blocking layer may include the heterocyclic compound.

In still another organic light emitting device, the organic material layer includes an electron transport layer, a light emitting layer or a hole blocking layer, and the electron transport layer, the light emitting layer or the hole blocking layer may include the heterocyclic compound.

The organic light emitting device of the present invention may further include one or two or more layers selected from the group consisting of a light emitting layer, a hole injection layer, a hole transport layer, an electron injection layer, an electron transport layer, an electron blocking layer, and a hole blocking layer.

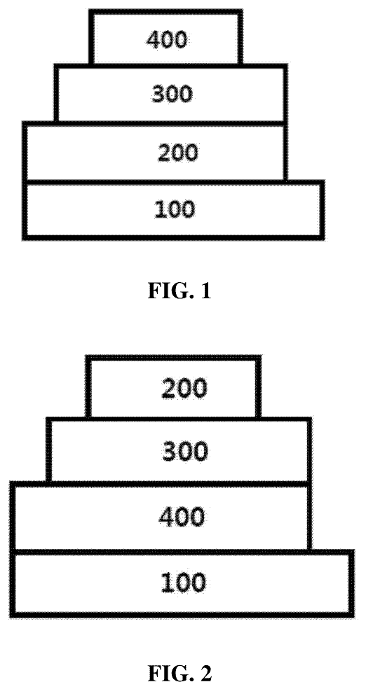

FIGS. 1 to 3 exemplify the stacking sequence of the electrodes and the organic material layer of the organic light emitting device according to an exemplary embodiment of the present application. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as illustrated in FIG. 2, an organic light emitting device in which a negative electrode, an organic material layer, and a positive electrode are sequentially stacked on a substrate may also be implemented.

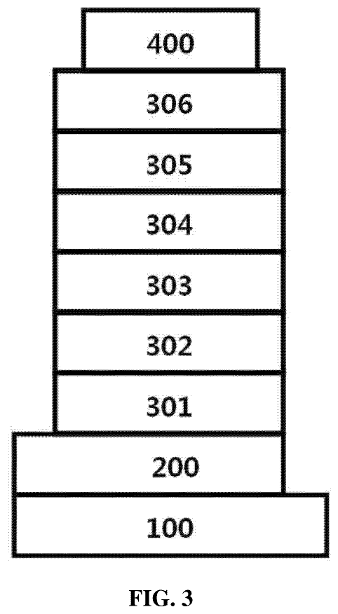

FIG. 3 exemplifies a case where an organic material layer is a multilayer. An organic light emitting device according to FIG. 3 includes a hole injection layer 301, a hole transport layer 302, a light emitting layer 303, a hole blocking layer 304, an electron transport layer 305, and an electron injection layer 306. However, the scope of the present application is not limited by the stacking structure as described above, and if necessary, the other layers except for the light emitting layer may be omitted, and another necessary functional layer may be further added.

In an exemplary embodiment of the present application, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for an organic material layer according to an exemplary embodiment of the present application.

In an exemplary embodiment of the present application, provided is a method for manufacturing an organic light emitting device, in which the forming of the organic material layer forms the organic material layer by pre-mixing the heterocyclic compound of Chemical Formula 1 and the heterocyclic compound of Chemical Formula 2 or 3, and using a thermal vacuum deposition method.

The pre-mixing means that before the heterocyclic compound of Chemical Formula 1 and the heterocyclic compound of Chemical Formula 2 or 3 are deposited onto an organic material layer, the materials are first mixed and the mixture is contained in one common container and mixed.

The pre-mixed material may be referred to as a composition for an organic material layer according to an exemplary embodiment of the present application.

An organic material layer including Chemical Formula 1 may additionally include another material, if necessary.

An organic material layer simultaneously including Chemical Formula 1 and Chemical Formula 2 or 3 may additionally include another material, if necessary.

In the organic light emitting device according to an exemplary embodiment of the present application, materials other than the compounds of Chemical Formulae 1 to 3 will be exemplified below, but these materials are illustrative only, and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited and used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, two or more types of materials selected from n-type host materials or p-type host materials may be used as a host material for a light emitting layer.

The organic light emitting device according to an exemplary embodiment of the present application may be a top emission type, a bottom emission type, or a dual emission type according to the material to be used.

The heterocyclic compound according to an exemplary embodiment of the present application may act even in organic electronic devices including organic solar cells, organic photoconductors, organic transistors, and the like, based on the principle similar to those applied to organic light emitting devices.

### MODE FOR INVENTION

Hereinafter, the present specification will be described in more detail through Examples, but these Examples are provided only for exemplifying the present application, and are not intended to limit the scope of the present application.

### PREPARATION EXAMPLES

### <Preparation Example 1> Preparation of Compound 1-1

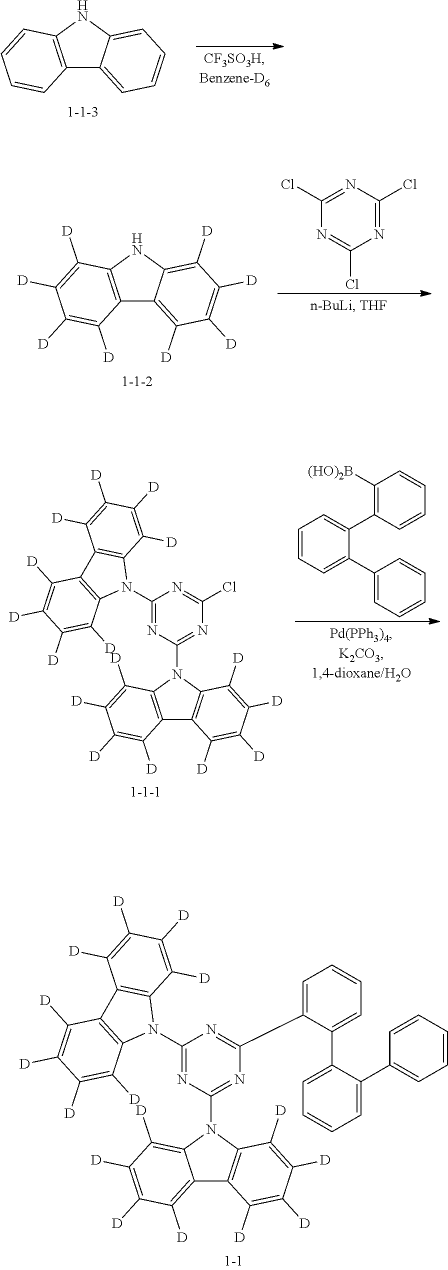 [SMILES: c1ccc2c(c1)[nH]c1ccccc12 ; [2H]c1c([2H])c([2H])c2c([nH]c3c([2H])c([2H])c([2H])c([2H])c32)c1[2H] ; Clc1nc(Cl)nc(Cl)n1 ; [2H]c1c([2H])c([2H])c2c(c1[2H])c1c([2H])c([2H])c([2H])c([2H])c1n2-c1nc(Cl)nc(-n2c3c([2H])c([2H])c([2H])c([2H])c3c3c([2H])c([2H])c([2H])c([2H])c32)n1 ; OB(O)c1ccccc1-c1ccccc1-c1ccccc1 ; [2H]c1c([2H])c([2H])c2c(c1[2H])c1c([2H])c([2H])c([2H])c([2H])c1n2-c1nc(-c2ccccc2-c2ccccc2-c2ccccc2)nc(-n2c3c([2H])c([2H])c([2H])c([2H])c3c3c([2H])c([2H])c([2H])c([2H])c32)n1]

### 1) Preparation of Compound 1-1-2

After 10 g (59.8 mM) of 9H-cabazole (1-1-3) was dissolved in 500 mL of benzene-d6, the resulting solution was dissolved in 170 g (1075 mM) of CF₃SO₃H, and refluxed at 60° C. for 1 hour. After the reaction was completed, the reaction product was neutralized with D₂O and Na₂CO₃. After neutralization, distilled water and ethyl acetate were added to the mixed solution, extraction was performed, the organic layer was dried over MgSO₄, and the solvent was removed using a rotary evaporator. The reaction product was purified by column chromatography (DCM:Hex=1:4) and methanol was used to obtain 8.22 g (78.4%) of Target Compound 1-1-2.

### 2) Preparation of Compound 1-1-1

8.22 g (46.9 mM) of Compound 1-1-2 was dissolved in THF and then nitrogen substitution was performed at −78° C. 22.5 mL (56.3 mM) of 2.5 M n-BuLi was slowly added thereto at −78° C., and the resulting mixture was stirred for 30 minutes. 5.5 g (23.5 mM) of 2,4,6-trichloro-1,3,5-triazine was added thereto, and the resulting mixture was stirred at room temperature for 2 hours. After the reaction was completed, distilled water and DCM were added thereto at room temperature, extraction was performed, the organic layer was dried over MgSO₄, and then the solvent was removed using a rotary evaporator. The reaction product was purified by recrystallization with methanol to obtain 10.8 g (78%) of Target Compound 1-1-1.

### 3) Preparation of Compound 1-1

After 10.8 g (23.4 mM) of Compound 1-1-1, 6.8 g (25.7 mM) of [1,1′: 2′,1″-terphenyl]-2-ylboronic acid, 1.3 g (1.1 mM) of Pd(PPh₃)₄, and 6.5 g (46.8 mM) of K₂CO₃ were dissolved in 250 mL/50 mL of 1,4-dioxane/H₂O, the resulting solution was refluxed for 24 hours. After the reaction was completed, distilled water and DCM were added thereto at room temperature, extraction was performed, the organic layer was dried over MgSO₄, and then the solvent was removed using a rotary evaporator. The reaction product was purified by column chromatography (DCM:Hex=1:3) and recrystallized with methanol to obtain 11.4 g (86%) of Target Compound 1-1.

The target compound was synthesized in the same manner as in the preparation of Preparation Example 1, except that Intermediate A in the following Table 1 was used instead of [1,1′: 2′,1″-terphenyl]-2-ylboronic acid in Preparation Example 1.

TABLE 1

| Com- | |
|---|---|
| pound | |
| No. | Intermediate A |
| 1-5 | 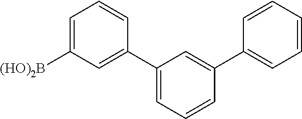 [SMILES: OB(O)c1cccc(-c2cccc(-c3ccccc3)c2)c1] |
| 1-9 | 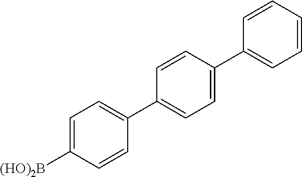 [SMILES: OB(O)c1ccc(-c2ccc(-c3ccccc3)cc2)cc1] |
| 1-40 | 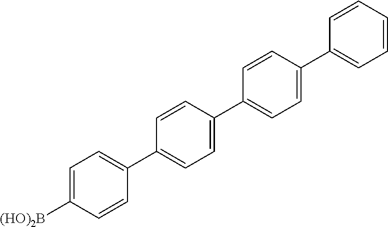 [SMILES: OB(O)c1ccc(-c2ccc(-c3ccc(-c4ccccc4)cc3)cc2)cc1] |
| 1-78 | 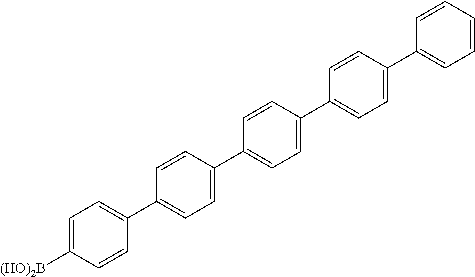 [SMILES: OB(O)c1ccc(-c2ccc(-c3ccc(-c4ccc(-c5ccccc5)cc4)cc3)cc2)cc1] |
| 1-79 | 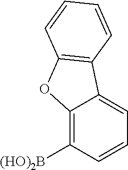 [SMILES: OB(O)c1cccc2c1oc1ccccc12] |
| 1-80 | 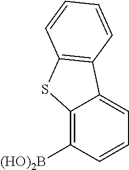 [SMILES: OB(O)c1cccc2c1sc1ccccc12] |
| 1-81 | 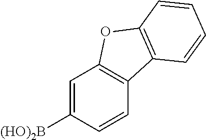 [SMILES: OB(O)c1ccc2c(c1)oc1ccccc12] |
| 1-88 | 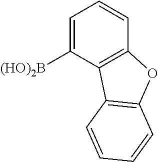 [SMILES: OB(O)c1cccc2oc3ccccc3c12] |
| 1-115 | 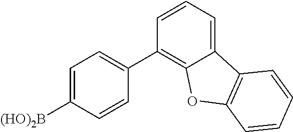 [SMILES: OB(O)c1ccc(-c2cccc3c2oc2ccccc23)cc1] |
| 1-117 | 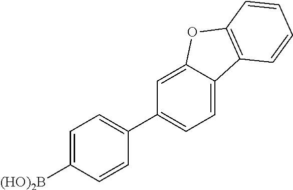 [SMILES: OB(O)c1ccc(-c2ccc3c(c2)oc2ccccc23)cc1] |
| 1-123 | 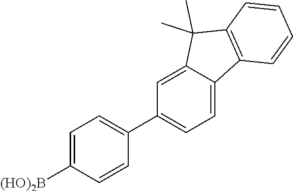 [SMILES: CC1(C)c2ccccc2-c2ccc(-c3ccc(B(O)O)cc3)cc21] |
| 1-398 | 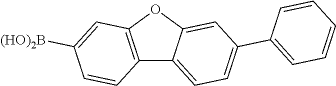 [SMILES: OB(O)c1ccc2c(c1)oc1cc(-c3ccccc3)ccc12] |
| 1-400 | 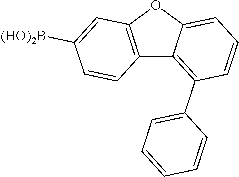 [SMILES: OB(O)c1ccc2c(c1)oc1cccc(-c3ccccc3)c12] |
| 1-404 | 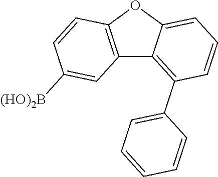 [SMILES: OB(O)c1ccc2oc3cccc(-c4ccccc4)c3c2c1] |
| 1-413 | 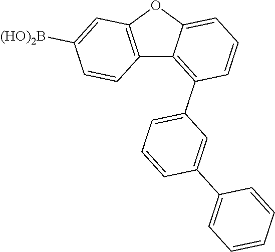 [SMILES: OB(O)c1ccc2c(c1)oc1cccc(-c3cccc(-c4ccccc4)c3)c12] |
| 1-432 | 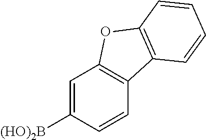 [SMILES: OB(O)c1ccc2c(c1)oc1ccccc12] |
| 1-433 | 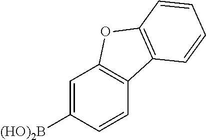 [SMILES: OB(O)c1ccc2c(c1)oc1ccccc12] |
| 1-434 | 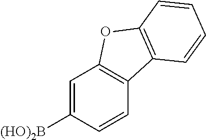 [SMILES: OB(O)c1ccc2c(c1)oc1ccccc12] |
| 1-435 | 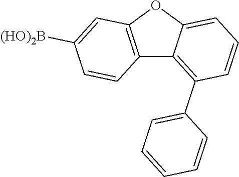 [SMILES: OB(O)c1ccc2c(c1)oc1cccc(-c3ccccc3)c12] |
| 1-436 | 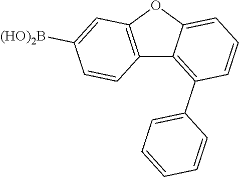 [SMILES: OB(O)c1ccc2c(c1)oc1cccc(-c3ccccc3)c12] |
| 1-437 | 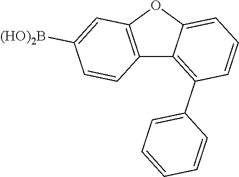 [SMILES: OB(O)c1ccc2c(c1)oc1cccc(-c3ccccc3)c12] |
| Com- | |
| pound | |
| No. | Target compound |
| 1-5 | 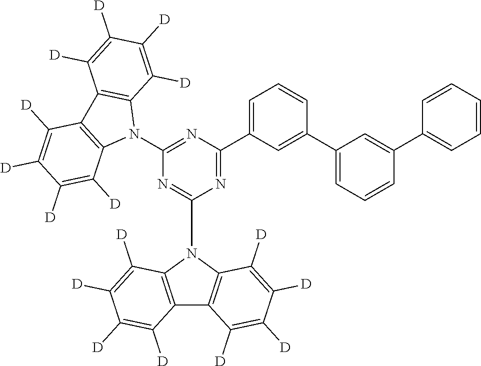 [SMILES: [2H]c1c([2H])c([2H])c2c(c1[2H])c1c([2H])c([2H])c([2H])c([2H])c1n2-c1nc(-c2cccc(-c3cccc(-c4ccccc4)c3)c2)nc(-n2c3c([2H])c([2H])c([2H])c([2H])c3c3c([2H])c([2H])c([2H])c([2H])c32)n1] |
| 1-9 | 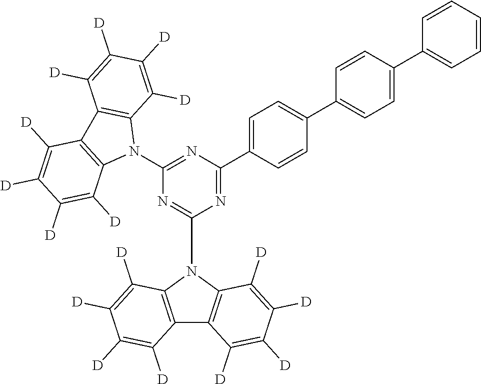 [SMILES: [2H]c1c([2H])c([2H])c2c(c1[2H])c1c([2H])c([2H])c([2H])c([2H])c1n2-c1nc(-c2ccc(-c3ccc(-c4ccccc4)cc3)cc2)nc(-n2c3c([2H])c([2H])c([2H])c([2H])c3c3c([2H])c([2H])c([2H])c([2H])c32)n1] |
| 1-40 | 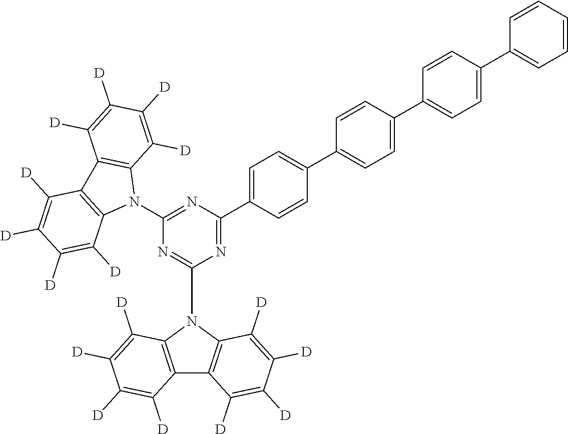 [SMILES: [2H]c1c([2H])c([2H])c2c(c1[2H])c1c([2H])c([2H])c([2H])c([2H])c1n2-c1nc(-c2ccc(-c3ccc(-c4ccc(-c5ccccc5)cc4)cc3)cc2)nc(-n2c3c([2H])c([2H])c([2H])c([2H])c3c3c([2H])c([2H])c([2H])c([2H])c32)n1] |
| 1-78 | 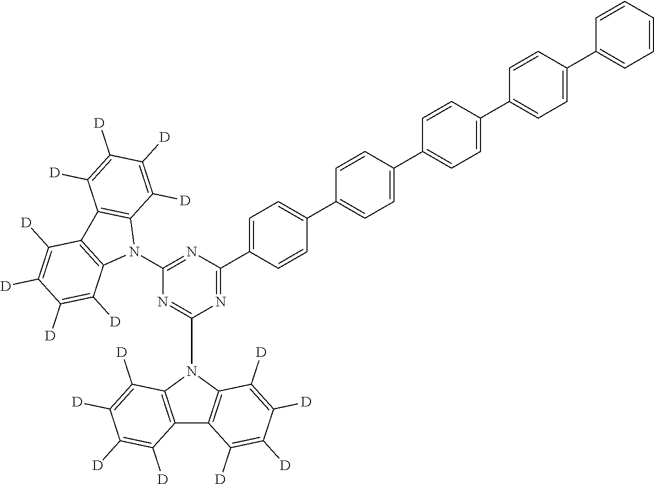 [SMILES: [2H]c1c([2H])c([2H])c2c(c1[2H])c1c([2H])c([2H])c([2H])c([2H])c1n2-c1nc(-c2ccc(-c3ccc(-c4ccc(-c5ccc(-c6ccccc6)cc5)cc4)cc3)cc2)nc(-n2c3c([2H])c([2H])c([2H])c([2H])c3c3c([2H])c([2H])c([2H])c([2H])c32)n1] |
| 1-79 | 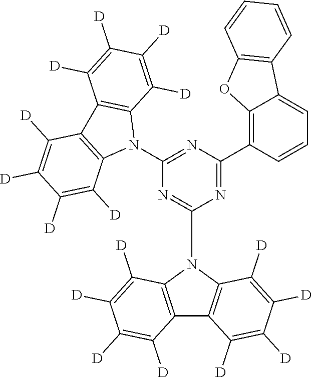 [SMILES: [2H]c1c([2H])c([2H])c2c(c1[2H])c1c([2H])c([2H])c([2H])c([2H])c1n2-c1nc(-c2cccc3c2oc2ccccc23)nc(-n2c3c([2H])c([2H])c([2H])c([2H])c3c3c([2H])c([2H])c([2H])c([2H])c32)n1] |
| 1-80 | 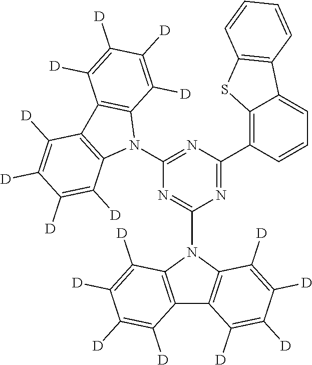 [SMILES: [2H]c1c([2H])c([2H])c2c(c1[2H])c1c([2H])c([2H])c([2H])c([2H])c1n2-c1nc(-c2cccc3c2sc2ccccc23)nc(-n2c3c([2H])c([2H])c([2H])c([2H])c3c3c([2H])c([2H])c([2H])c([2H])c32)n1] |
| 1-81 | 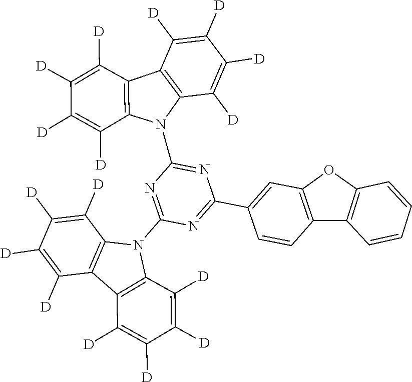 [SMILES: [2H]c1c([2H])c([2H])c2c(c1[2H])c1c([2H])c([2H])c([2H])c([2H])c1n2-c1nc(-c2ccc3c(c2)oc2ccccc23)nc(-n2c3c([2H])c([2H])c([2H])c([2H])c3c3c([2H])c([2H])c([2H])c([2H])c32)n1] |
| 1-88 | 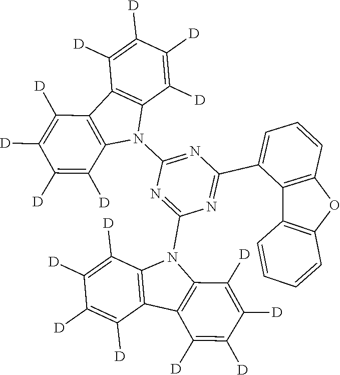 [SMILES: [2H]c1c([2H])c([2H])c2c(c1[2H])c1c([2H])c([2H])c([2H])c([2H])c1n2-c1nc(-c2cccc3oc4ccccc4c23)nc(-n2c3c([2H])c([2H])c([2H])c([2H])c3c3c([2H])c([2H])c([2H])c([2H])c32)n1] |
| 1-115 | 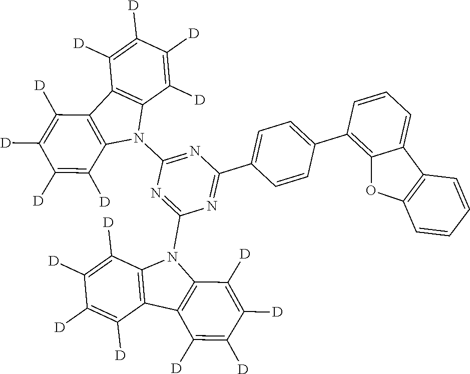 [SMILES: [2H]c1c([2H])c([2H])c2c(c1[2H])c1c([2H])c([2H])c([2H])c([2H])c1n2-c1nc(-c2ccc(-c3cccc4c3oc3ccccc34)cc2)nc(-n2c3c([2H])c([2H])c([2H])c([2H])c3c3c([2H])c([2H])c([2H])c([2H])c32)n1] |
| 1-117 | 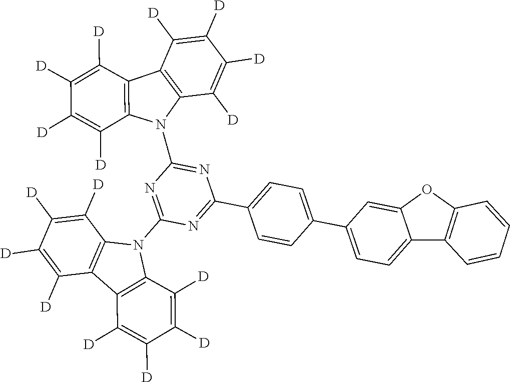 [SMILES: [2H]c1c([2H])c([2H])c2c(c1[2H])c1c([2H])c([2H])c([2H])c([2H])c1n2-c1nc(-c2ccc(-c3ccc4c(c3)oc3ccccc34)cc2)nc(-n2c3c([2H])c([2H])c([2H])c([2H])c3c3c([2H])c([2H])c([2H])c([2H])c32)n1] |
| 1-123 | 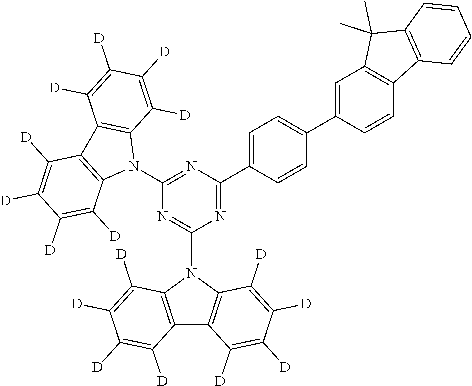 [SMILES: [2H]c1c([2H])c([2H])c2c(c1[2H])c1c([2H])c([2H])c([2H])c([2H])c1n2-c1nc(-c2ccc(-c3ccc4c(c3)C(C)(C)c3ccccc3-4)cc2)nc(-n2c3c([2H])c([2H])c([2H])c([2H])c3c3c([2H])c([2H])c([2H])c([2H])c32)n1] |
| 1-398 | 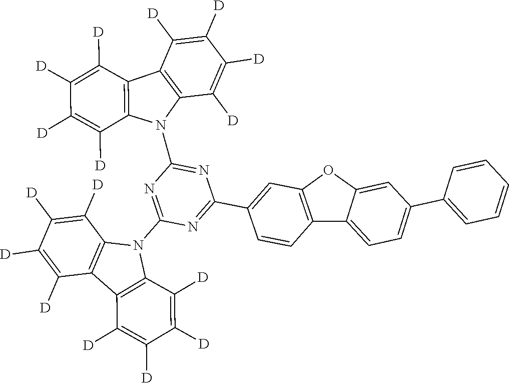 [SMILES: [2H]c1c([2H])c([2H])c2c(c1[2H])c1c([2H])c([2H])c([2H])c([2H])c1n2-c1nc(-c2ccc3c(c2)oc2cc(-c4ccccc4)ccc23)nc(-n2c3c([2H])c([2H])c([2H])c([2H])c3c3c([2H])c([2H])c([2H])c([2H])c32)n1] |
| 1-400 | 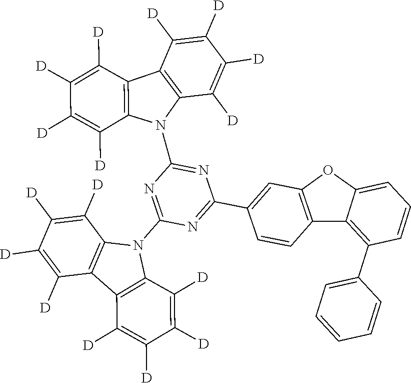 [SMILES: [2H]c1c([2H])c([2H])c2c(c1[2H])c1c([2H])c([2H])c([2H])c([2H])c1n2-c1nc(-c2ccc3c(c2)oc2cccc(-c4ccccc4)c23)nc(-n2c3c([2H])c([2H])c([2H])c([2H])c3c3c([2H])c([2H])c([2H])c([2H])c32)n1] |
| 1-404 | 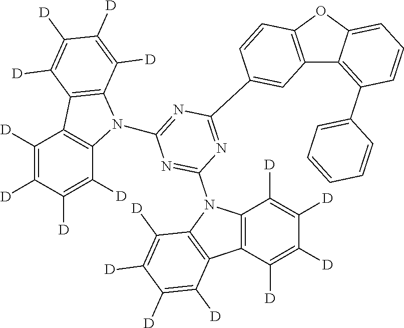 [SMILES: [2H]c1c([2H])c([2H])c2c(c1[2H])c1c([2H])c([2H])c([2H])c([2H])c1n2-c1nc(-c2ccc3oc4cccc(-c5ccccc5)c4c3c2)nc(-n2c3c([2H])c([2H])c([2H])c([2H])c3c3c([2H])c([2H])c([2H])c([2H])c32)n1] |
| 1-413 | 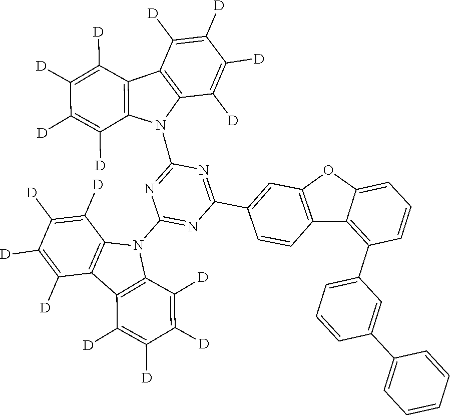 [SMILES: [2H]c1c([2H])c([2H])c2c(c1[2H])c1c([2H])c([2H])c([2H])c([2H])c1n2-c1nc(-c2ccc3c(c2)oc2cccc(-c4cccc(-c5ccccc5)c4)c23)nc(-n2c3c([2H])c([2H])c([2H])c([2H])c3c3c([2H])c([2H])c([2H])c([2H])c32)n1] |
| 1-432 | 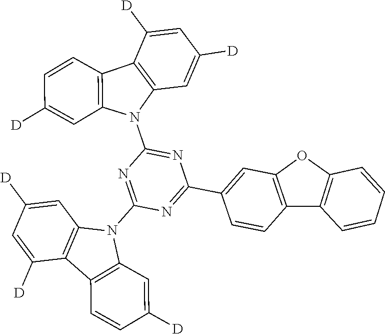 [SMILES: [2H]c1ccc2c3c([2H])cc([2H])cc3n(-c3nc(-c4ccc5c(c4)oc4ccccc45)nc(-n4c5cc([2H])ccc5c5c([2H])cc([2H])cc54)n3)c2c1] |
| 1-433 | 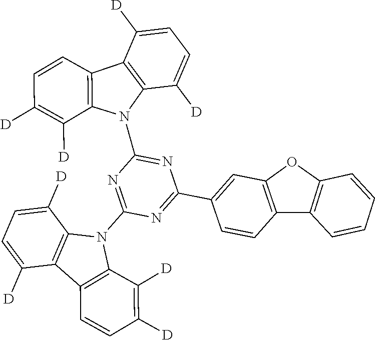 [SMILES: [2H]c1ccc2c3c([2H])ccc([2H])c3n(-c3nc(-c4ccc5c(c4)oc4ccccc45)nc(-n4c5c([2H])c([2H])ccc5c5c([2H])ccc([2H])c54)n3)c2c1[2H]] |
| 1-434 | 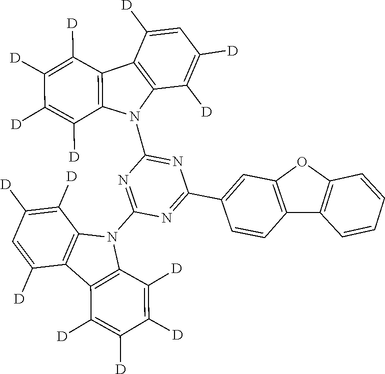 [SMILES: [2H]c1cc([2H])c2c3c([2H])c([2H])c([2H])c([2H])c3n(-c3nc(-c4ccc5c(c4)oc4ccccc45)nc(-n4c5c([2H])c([2H])cc([2H])c5c5c([2H])c([2H])c([2H])c([2H])c54)n3)c2c1[2H]] |
| 1-435 | 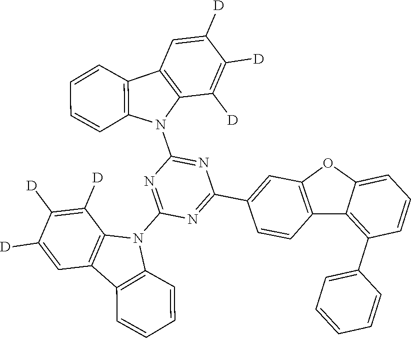 [SMILES: [2H]c1cc2c3ccccc3n(-c3nc(-c4ccc5c(c4)oc4cccc(-c6ccccc6)c45)nc(-n4c5ccccc5c5cc([2H])c([2H])c([2H])c54)n3)c2c([2H])c1[2H]] |
| 1-436 | 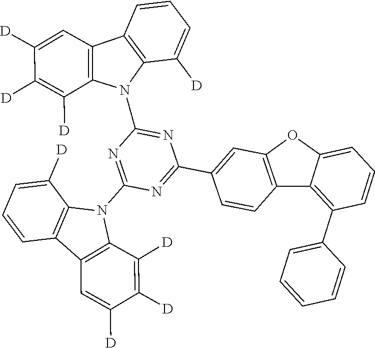 [SMILES: [2H]c1cc2c3cccc([2H])c3n(-c3nc(-c4ccc5c(c4)oc4cccc(-c6ccccc6)c45)nc(-n4c5c([2H])cccc5c5cc([2H])c([2H])c([2H])c54)n3)c2c([2H])c1[2H]] |
| 1-437 | 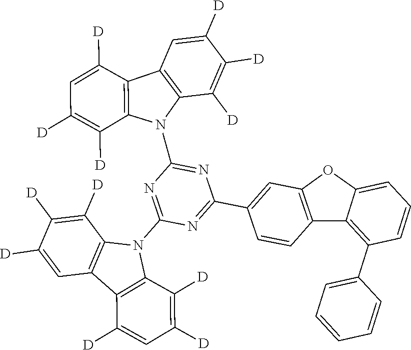 [SMILES: [2H]c1cc2c3c([2H])cc([2H])c([2H])c3n(-c3nc(-c4ccc5c(c4)oc4cccc(-c6ccccc6)c45)nc(-n4c5c([2H])c([2H])c([2H])cc5c5c([2H])cc([2H])c([2H])c54)n3)c2c([2H])c1[2H]] |

### <Preparation Example 2> Preparation of Compound 1-177

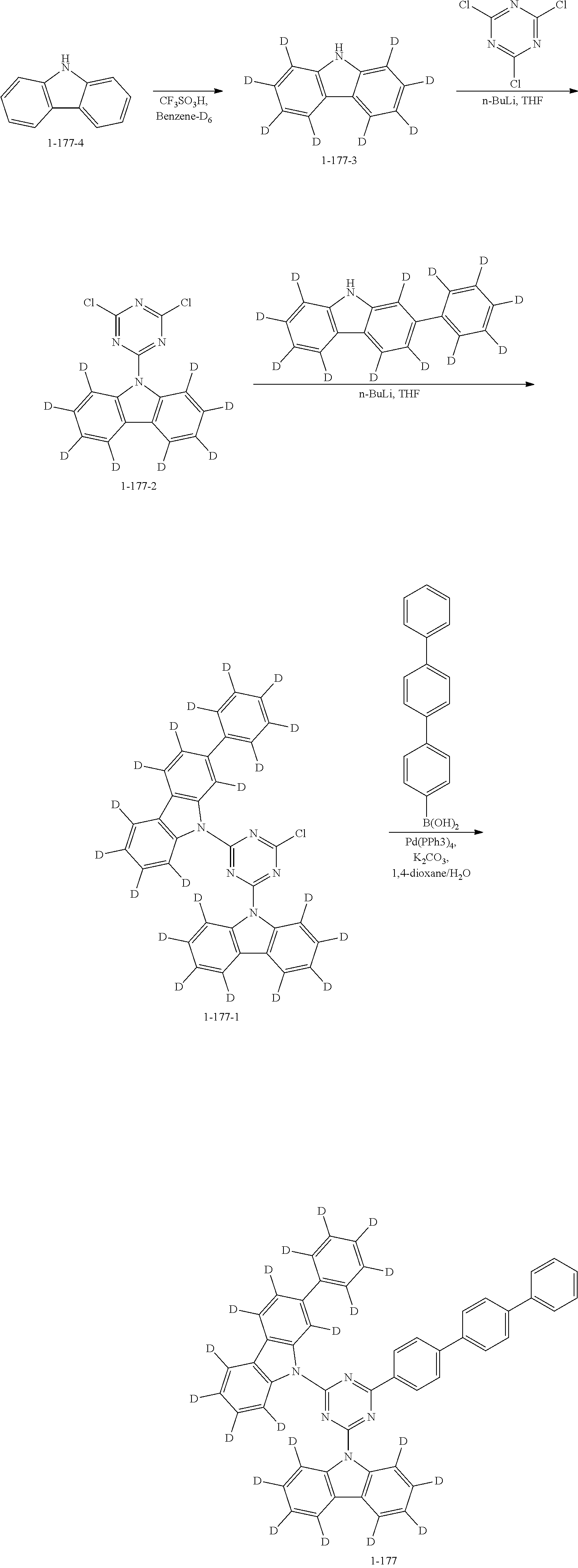

### 1) Preparation of Compound 1-177-3

After 10 g (59.8 mM) of 9H-cabazole was dissolved in 500 mL of benzene-d6, the resulting solution was dissolved in 170 g (1075 mM) of CF₃SO₃H, and refluxed at 60° C. for 1 hour. After the reaction was completed, the reaction product was neutralized with D₂O and Na₂CO₃. After neutralization, distilled water and ethyl acetate were added to the mixed solution, extraction was performed, the organic layer was dried over MgSO₄, and the solvent was removed using a rotary evaporator. The reaction product was purified by column chromatography (DCM:Hex=1:4) and methanol was used to obtain 8.22 g (78.4%) of Target Compound 1-177-3.

### 2) Preparation of Compound 1-177-2

8.22 g (46.9 mM) of Compound 1-177-3 was dissolved in THF and then nitrogen substitution was performed at −78° C. 22.5 mL (56.3 mM) of 2.5 M n-BuLi was slowly added thereto at −78° C., and the resulting mixture was stirred for 30 minutes. 8.65 g (46.9 mM) of 2,4,6-trichloro-1,3,5-triazine was added thereto, and the resulting mixture was stirred at room temperature for 2 hours. After the reaction was completed, distilled water and DCM were added thereto at room temperature, extraction was performed, the organic layer was dried over MgSO₄, and then the solvent was removed using a rotary evaporator. The reaction product was purified by recrystallization with methanol to obtain 12.3 g (81%) of Target Compound 1-177-2.

### 3) Preparation of Compound 1-177-1

After 9.73 g (38.1 mM) of 2-(phenyl-d₅)-9H-carbazole-1,3,4,5,6,7,8-d₇) was dissolved in THF, nitrogen substitution was performed at −78° C. 22.9 mL (57.2 mM) of 2.5 M n-BuLi was slowly added thereto at −78° C., and the resulting mixture was stirred for 30 minutes. 12.3 g (38.1 mM) of Compound 1-177-2 was added thereto, and the resulting mixture was stirred at room temperature for 2 hours. After the reaction was completed, distilled water and DCM were added thereto at room temperature, extraction was performed, the organic layer was dried over MgSO₄, and then the solvent was removed using a rotary evaporator. The reaction product was purified by recrystallization with methanol to obtain 17.3 g (84%) of Target Compound 1-177-1.

### 4) Preparation of Compound 1-177

After 17.3 g (31.9 mM) of Compound 1-177-1, 9.6 g (35.1 mM) of [1,1′: 2′,1″-terphenyl]-4-ylboronic acid, 1.8 g (1.6 mM) of Pd(PPh₃)₄, and 8.82 g (63.8 mM) of K₂CO₃ were dissolved in 500 mL/100 mL of 1,4-dioxane/H₂O, the resulting solution was refluxed for 24 hours. After the reaction was completed, distilled water and DCM were added thereto at room temperature, extraction was performed, the organic layer was dried over MgSO₄, and then the solvent was removed using a rotary evaporator. The reaction product was purified by column chromatography (DCM:Hex=1:3) and recrystallized with methanol to obtain 13.6 g (85%) of Target Compound 1-177.

The target compound was synthesized in the same manner as in the preparation of Preparation Example 2, except that Intermediate A in the following Table 2 was used instead of [1,1′: 2′,1″-terphenyl]-4-ylboronic acid in Preparation Example 2.

TABLE 2

| Com- | |
|---|---|
| pound | |
| No. | Intermediate A |
| 1-208 | 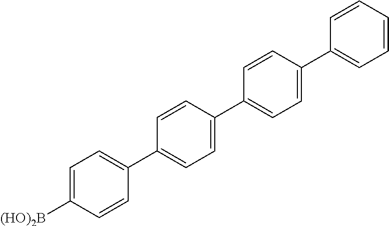 [SMILES: OB(O)c1ccc(-c2ccc(-c3ccc(-c4ccccc4)cc3)cc2)cc1] |
| 1-211 | 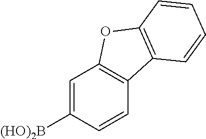 [SMILES: OB(O)c1ccc2c(c1)oc1ccccc12] |
| Com- | |
| pound | |
| No. | Target compound |
| 1-208 | 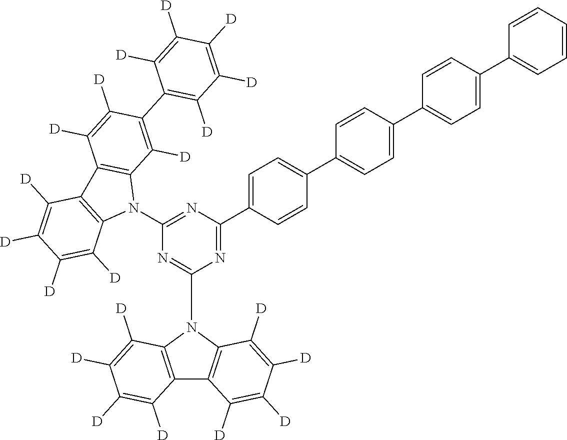 [SMILES: [2H]c1c([2H])c([2H])c(-c2c([2H])c([2H])c3c4c([2H])c([2H])c([2H])c([2H])c4n(-c4nc(-c5ccc(-c6ccc(-c7ccc(-c8ccccc8)cc7)cc6)cc5)nc(-n5c6c([2H])c([2H])c([2H])c([2H])c6c6c([2H])c([2H])c([2H])c([2H])c65)n4)c3c2[2H])c([2H])c1[2H]] |
| 1-211 | 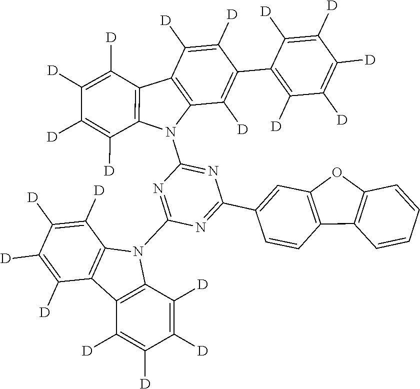 [SMILES: [2H]c1c([2H])c([2H])c(-c2c([2H])c([2H])c3c4c([2H])c([2H])c([2H])c([2H])c4n(-c4nc(-c5ccc6c(c5)oc5ccccc56)nc(-n5c6c([2H])c([2H])c([2H])c([2H])c6c6c([2H])c([2H])c([2H])c([2H])c65)n4)c3c2[2H])c([2H])c1[2H]] |

The target compound was synthesized in the same manner as in the preparation of Preparation Example 1, except that Intermediate A and Intermediate B in the following Table 3 were used instead of 9H-carbazole and [1,1′: 2′,1″-terphenyl]-2-ylboronic acid, respectively, in Preparation Example 2.

TABLE 3

| Com- | | |
|---|---|---|
| pound | | |
| No. | Intermediate A | Intermediate B |
| 1-298 | 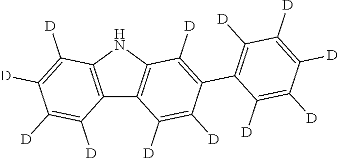 [SMILES: [2H]c1c([2H])c([2H])c(-c2c([2H])c([2H])c3c([nH]c4c([2H])c([2H])c([2H])c([2H])c43)c2[2H])c([2H])c1[2H]] | 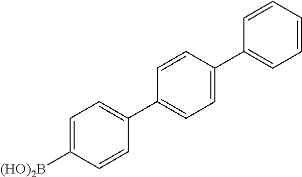 [SMILES: OB(O)c1ccc(-c2ccc(-c3ccccc3)cc2)cc1] |
| 1-301 | 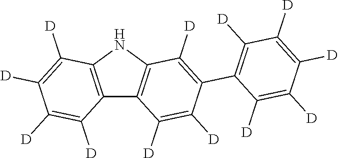 [SMILES: [2H]c1c([2H])c([2H])c(-c2c([2H])c([2H])c3c([nH]c4c([2H])c([2H])c([2H])c([2H])c43)c2[2H])c([2H])c1[2H]] | 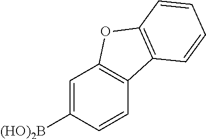 [SMILES: OB(O)c1ccc2c(c1)oc1ccccc12] |

| Com- | |
|---|---|
| pound | |
| No. | Target compound |
| 1-298 | 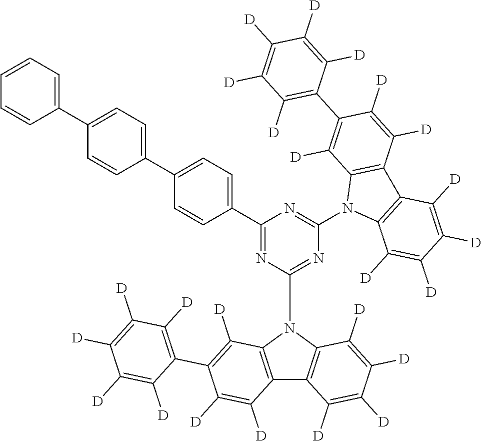 [SMILES: [2H]c1c([2H])c([2H])c(-c2c([2H])c([2H])c3c4c([2H])c([2H])c([2H])c([2H])c4n(-c4nc(-c5ccc(-c6ccc(-c7ccccc7)cc6)cc5)nc(-n5c6c([2H])c([2H])c([2H])c([2H])c6c6c([2H])c([2H])c(-c7c([2H])c([2H])c([2H])c([2H])c7[2H])c([2H])c65)n4)c3c2[2H])c([2H])c1[2H]] |
| 1-301 | 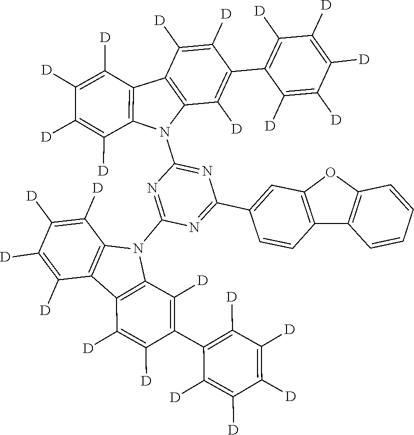 [SMILES: [2H]c1c([2H])c([2H])c(-c2c([2H])c([2H])c3c4c([2H])c([2H])c([2H])c([2H])c4n(-c4nc(-c5ccc6c(c5)oc5ccccc56)nc(-n5c6c([2H])c([2H])c([2H])c([2H])c6c6c([2H])c([2H])c(-c7c([2H])c([2H])c([2H])c([2H])c7[2H])c([2H])c65)n4)c3c2[2H])c([2H])c1[2H]] |

### <Preparation Example 3> Preparation of Compound 2-1

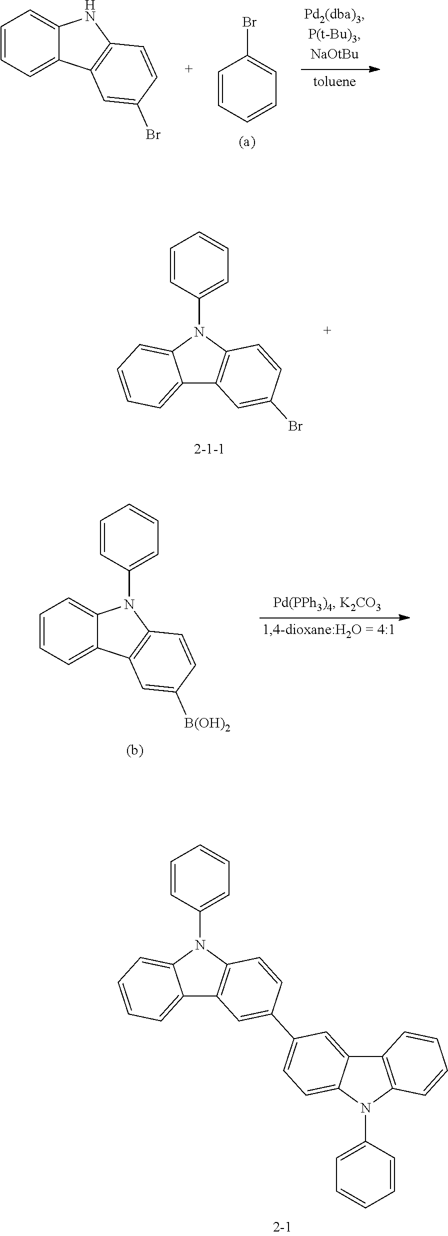 [SMILES: Brc1ccc2[nH]c3ccccc3c2c1 ; Brc1ccccc1 ; Brc1ccc2c(c1)c1ccccc1n2-c1ccccc1 ; OB(O)c1ccc2c(c1)c1ccccc1n2-c1ccccc1 ; c1ccc(-n2c3ccccc3c3cc(-c4ccc5c(c4)c4ccccc4n5-c4ccccc4)ccc32)cc1]

### 1) Preparation of Compound 2-1-1

After 10 g (49.59 mmol) of 3-bromo-9H-carbazole, 24.2 g (148.77 mmol) of 2-bromobenzene-1-ylium (a), 2.27 g (2.48 mmol) of tris(dibenzylideneacetone)dipalladium (Pd₂(dba)₃), 2.42 mL (9.92 mmol) of tri-tert-butylphosphine (P(t-Bu)₃), and 9.53 g (99.18 mmol) of sodium tert-butoxide (NatOBu) were put into a reaction flask, 100 mL of toluene was added thereto, and the resulting mixture was heated at 135° C. for 15 hours. When the reaction was terminated, the resulting product was extracted with methylene chloride (MC) and water, and then purified by column chromatography to obtain 14 g of Compound 2-1-1 (yield 98%).

### 2) Preparation of Compound 2-1

After 14 g (43.4 mmol) of Compound 2-1-1, 14.9 g (52 mmol) of (9-phenyl-9H-carbazol-3-yl)boronic acid (b), 2.5 g (2.17 mmol) of tetrakis(triphenylphosphine)palladium(0) (Pd(PPh₃)₄), and 17.9 g (130 mmol) of potassium carbonate (K₂CO₃) were put into a reaction flask, 140 mL of 1,4-dioxane and 35 mL of distilled water were added thereto, and the resulting mixture was stirred at 120° C. for 4 hours.

Thereafter, a solid produced by lowering the temperature to room temperature was washed with distilled water and methanol to obtain 17 g (yield 80%) of Compound 2-1.

### <Preparation Example 4> Preparation of Compounds 2-2, 2-3, 2-4, 2-5, 2-6, 2-7, 2-11, 2-16, 2-19, 2-20, 2-21, 2-22, 2-23, 2-26, 2-27, 2-28, 2-29, 2-30, 2-32, 2-33, 2-34, 2-38, 2-40, 2-41, 2-42, 2-43, 2-45, 2-46, 2-48, 2-49, 2-50, 2-51, 2-52, 2-55, 2-57, and 2-60

Compounds 2-2, 2-3, 2-4, 2-5, 2-6, 2-7, 2-11, 2-16, 2-19, 2-20, 2-21, 2-22, 2-23, 2-26, 2-27, 2-28, 2-29, 2-30, 2-32, 2-33, 2-34, 2-38, 2-40, 2-41, 2-42, 2-43, 2-45, 2-46, 2-48, 2-49, 2-50, 2-51, 2-52, 2-55, 2-57 and 2-60 were synthesized in the same manner as in the preparation of Preparation Example 3, except that Compound a and Compound b in the following Table 4 were used instead of 2-bromobenzene-1-ylium (a) and (9-phenyl-9H-carbazol-3-yl)boronic acid (b), respectively, in Preparation Example 3.

TABLE 4

| Com- | | |
|---|---|---|
| pound | | |
| No. | Compound a | Compound b |
| 2-2 | 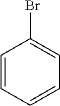 | 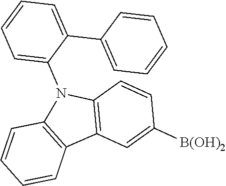 [SMILES: OB(O)c1ccc2c(c1)c1ccccc1n2-c1ccccc1-c1ccccc1] |
| 2-3 | 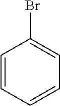 | 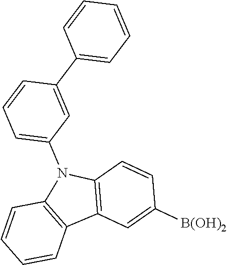 [SMILES: OB(O)c1ccc2c(c1)c1ccccc1n2-c1cccc(-c2ccccc2)c1] |
| 2-4 | 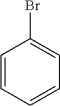 | 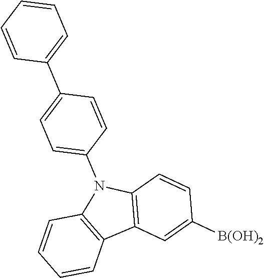 [SMILES: OB(O)c1ccc2c(c1)c1ccccc1n2-c1ccc(-c2ccccc2)cc1] |
| 2-5 | 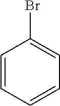 | 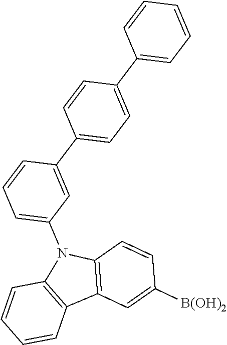 [SMILES: OB(O)c1ccc2c(c1)c1ccccc1n2-c1cccc(-c2ccc(-c3ccccc3)cc2)c1] |
| 2-6 | 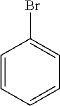 | 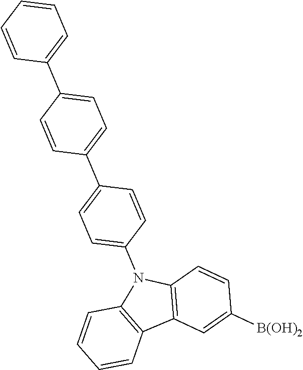 [SMILES: OB(O)c1ccc2c(c1)c1ccccc1n2-c1ccc(-c2ccc(-c3ccccc3)cc2)cc1] |
| 2-7 | 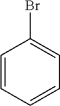 | 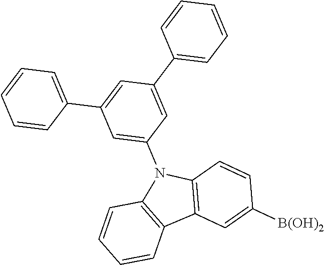 [SMILES: OB(O)c1ccc2c(c1)c1ccccc1n2-c1cc(-c2ccccc2)cc(-c2ccccc2)c1] |
| 2-11 | 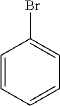 | 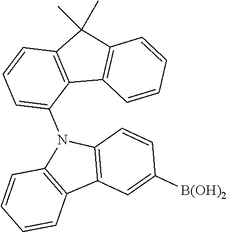 [SMILES: CC1(C)c2ccccc2-c2c(-n3c4ccccc4c4cc(B(O)O)ccc43)cccc21] |
| 2-16 | 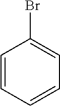 | 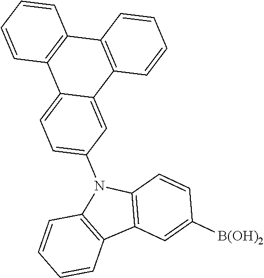 [SMILES: OB(O)c1ccc2c(c1)c1ccccc1n2-c1ccc2c3ccccc3c3ccccc3c2c1] |
| 2-19 | 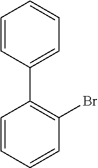 [SMILES: Brc1ccccc1-c1ccccc1] | 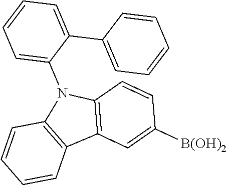 [SMILES: OB(O)c1ccc2c(c1)c1ccccc1n2-c1ccccc1-c1ccccc1] |
| 2-20 | 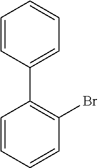 [SMILES: Brc1ccccc1-c1ccccc1] | 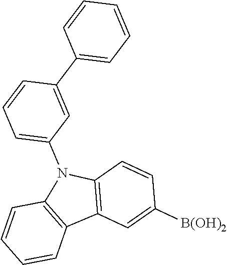 [SMILES: OB(O)c1ccc2c(c1)c1ccccc1n2-c1cccc(-c2ccccc2)c1] |
| 2-21 | 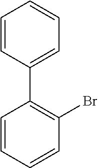 [SMILES: Brc1ccccc1-c1ccccc1] | 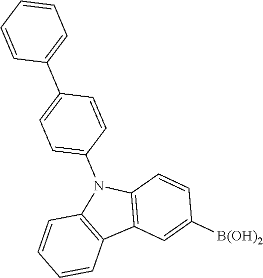 [SMILES: OB(O)c1ccc2c(c1)c1ccccc1n2-c1ccc(-c2ccccc2)cc1] |
| 2-22 | 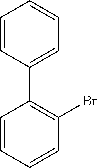 [SMILES: Brc1ccccc1-c1ccccc1] | 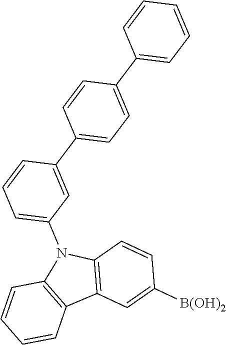 [SMILES: OB(O)c1ccc2c(c1)c1ccccc1n2-c1cccc(-c2ccc(-c3ccccc3)cc2)c1] |
| 2-23 | 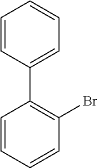 [SMILES: Brc1ccccc1-c1ccccc1] | 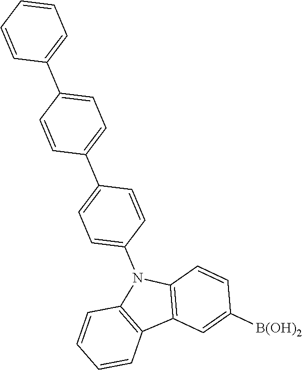 [SMILES: OB(O)c1ccc2c(c1)c1ccccc1n2-c1ccc(-c2ccc(-c3ccccc3)cc2)cc1] |
| 2-26 | 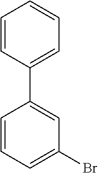 [SMILES: Brc1cccc(-c2ccccc2)c1] | 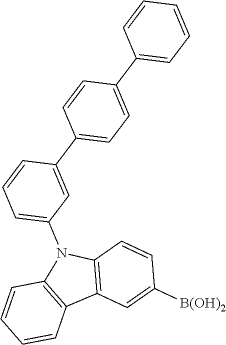 [SMILES: OB(O)c1ccc2c(c1)c1ccccc1n2-c1cccc(-c2ccc(-c3ccccc3)cc2)c1] |
| 2-27 | 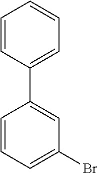 [SMILES: Brc1cccc(-c2ccccc2)c1] | 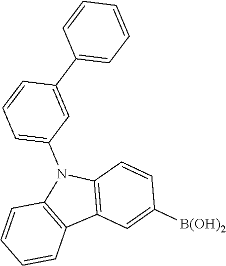 [SMILES: OB(O)c1ccc2c(c1)c1ccccc1n2-c1cccc(-c2ccccc2)c1] |
| 2-28 | 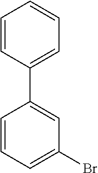 [SMILES: Brc1cccc(-c2ccccc2)c1] | 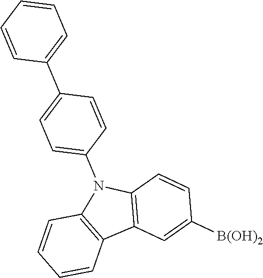 [SMILES: OB(O)c1ccc2c(c1)c1ccccc1n2-c1ccc(-c2ccccc2)cc1] |
| 2-29 | 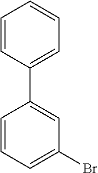 [SMILES: Brc1cccc(-c2ccccc2)c1] | 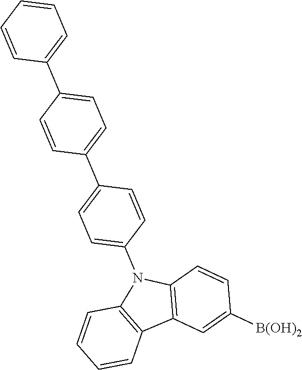 [SMILES: OB(O)c1ccc2c(c1)c1ccccc1n2-c1ccc(-c2ccc(-c3ccccc3)cc2)cc1] |
| 2-30 | 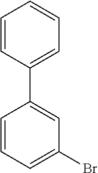 [SMILES: Brc1cccc(-c2ccccc2)c1] | 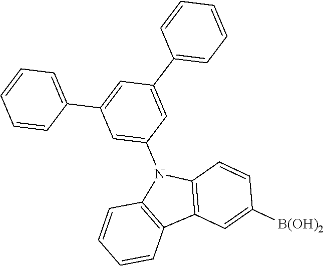 [SMILES: OB(O)c1ccc2c(c1)c1ccccc1n2-c1cc(-c2ccccc2)cc(-c2ccccc2)c1] |
| 2-32 | 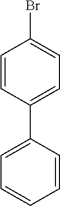 [SMILES: Brc1ccc(-c2ccccc2)cc1] | 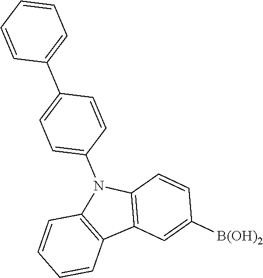 [SMILES: OB(O)c1ccc2c(c1)c1ccccc1n2-c1ccc(-c2ccccc2)cc1] |
| 2-33 | 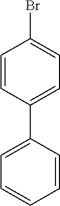 [SMILES: Brc1ccc(-c2ccccc2)cc1] | 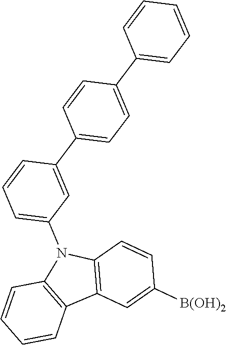 [SMILES: OB(O)c1ccc2c(c1)c1ccccc1n2-c1cccc(-c2ccc(-c3ccccc3)cc2)c1] |
| 2-34 | 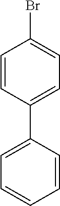 [SMILES: Brc1ccc(-c2ccccc2)cc1] | 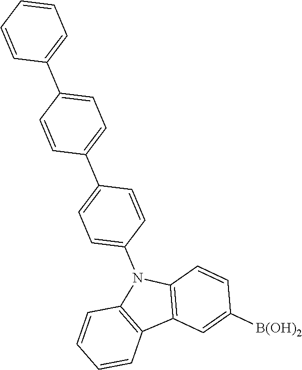 [SMILES: OB(O)c1ccc2c(c1)c1ccccc1n2-c1ccc(-c2ccc(-c3ccccc3)cc2)cc1] |
| 2-38 | 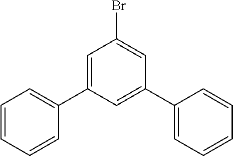 [SMILES: Brc1cc(-c2ccccc2)cc(-c2ccccc2)c1] | 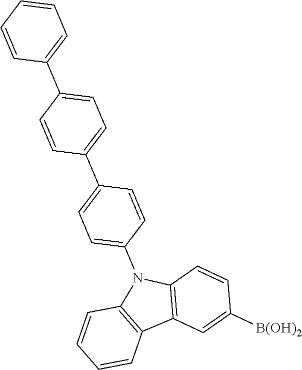 [SMILES: OB(O)c1ccc2c(c1)c1ccccc1n2-c1ccc(-c2ccc(-c3ccccc3)cc2)cc1] |
| 2-40 | 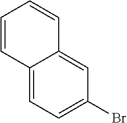 [SMILES: Brc1ccc2ccccc2c1] | 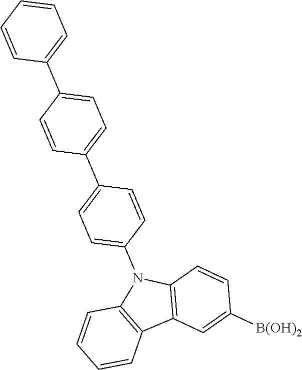 [SMILES: OB(O)c1ccc2c(c1)c1ccccc1n2-c1ccc(-c2ccc(-c3ccccc3)cc2)cc1] |
| 2-41 | 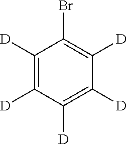 [SMILES: [2H]c1c([2H])c([2H])c(Br)c([2H])c1[2H]] | 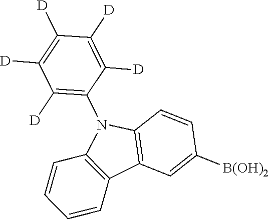 [SMILES: [2H]c1c([2H])c([2H])c(-n2c3ccccc3c3cc(B(O)O)ccc32)c([2H])c1[2H]] |
| 2-42 | 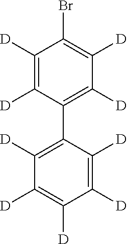 [SMILES: [2H]c1c([2H])c([2H])c(-c2c([2H])c([2H])c(Br)c([2H])c2[2H])c([2H])c1[2H]] | 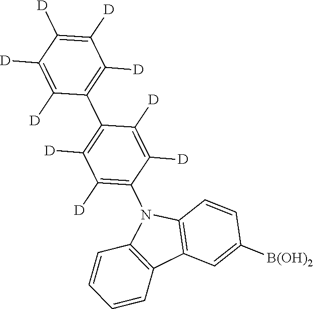 [SMILES: [2H]c1c([2H])c([2H])c(-c2c([2H])c([2H])c(-n3c4ccccc4c4cc(B(O)O)ccc43)c([2H])c2[2H])c([2H])c1[2H]] |
| 2-43 | 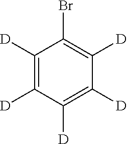 [SMILES: [2H]c1c([2H])c([2H])c(Br)c([2H])c1[2H]] | 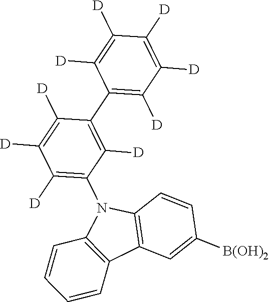 [SMILES: [2H]c1c([2H])c([2H])c(-c2c([2H])c([2H])c([2H])c(-n3c4ccccc4c4cc(B(O)O)ccc43)c2[2H])c([2H])c1[2H]] |
| 2-45 | 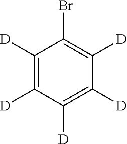 [SMILES: [2H]c1c([2H])c([2H])c(Br)c([2H])c1[2H]] | 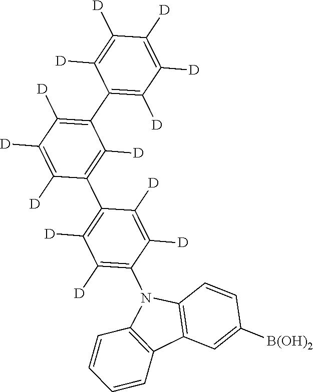 [SMILES: [2H]c1c([2H])c([2H])c(-c2c([2H])c([2H])c([2H])c(-c3c([2H])c([2H])c(-n4c5ccccc5c5cc(B(O)O)ccc54)c([2H])c3[2H])c2[2H])c([2H])c1[2H]] |
| 2-46 | 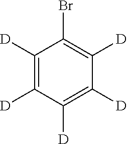 [SMILES: [2H]c1c([2H])c([2H])c(Br)c([2H])c1[2H]] | 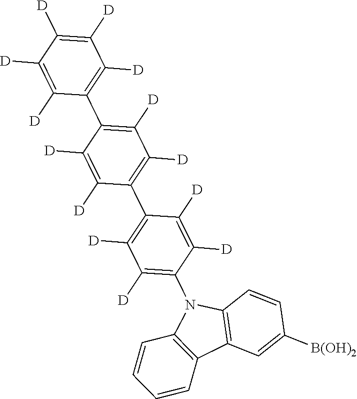 [SMILES: [2H]c1c([2H])c([2H])c(-c2c([2H])c([2H])c(-c3c([2H])c([2H])c(-n4c5ccccc5c5cc(B(O)O)ccc54)c([2H])c3[2H])c([2H])c2[2H])c([2H])c1[2H]] |
| 2-48 | 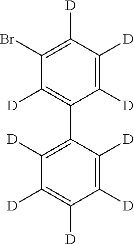 [SMILES: [2H]c1c([2H])c([2H])c(-c2c([2H])c([2H])c([2H])c(Br)c2[2H])c([2H])c1[2H]] | 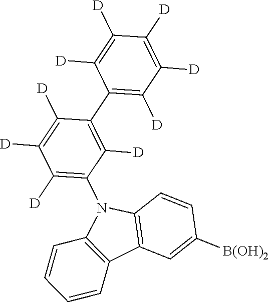 [SMILES: [2H]c1c([2H])c([2H])c(-c2c([2H])c([2H])c([2H])c(-n3c4ccccc4c4cc(B(O)O)ccc43)c2[2H])c([2H])c1[2H]] |
| 2-49 | 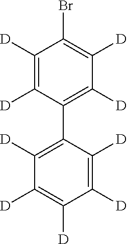 [SMILES: [2H]c1c([2H])c([2H])c(-c2c([2H])c([2H])c(Br)c([2H])c2[2H])c([2H])c1[2H]] | 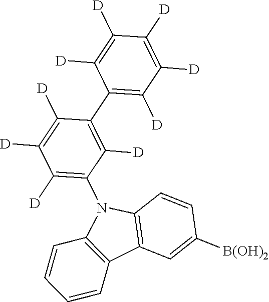 [SMILES: [2H]c1c([2H])c([2H])c(-c2c([2H])c([2H])c([2H])c(-n3c4ccccc4c4cc(B(O)O)ccc43)c2[2H])c([2H])c1[2H]] |
| 2-50 | 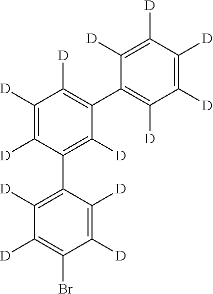 [SMILES: [2H]c1c([2H])c([2H])c(-c2c([2H])c([2H])c([2H])c(-c3c([2H])c([2H])c(Br)c([2H])c3[2H])c2[2H])c([2H])c1[2H]] | 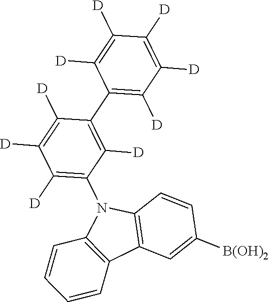 [SMILES: [2H]c1c([2H])c([2H])c(-c2c([2H])c([2H])c([2H])c(-n3c4ccccc4c4cc(B(O)O)ccc43)c2[2H])c([2H])c1[2H]] |
| 2-51 | 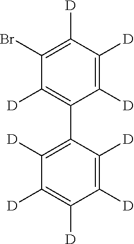 [SMILES: [2H]c1c([2H])c([2H])c(-c2c([2H])c([2H])c([2H])c(Br)c2[2H])c([2H])c1[2H]] | 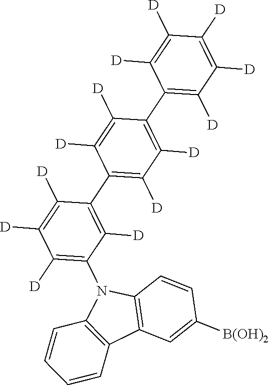 [SMILES: [2H]c1c([2H])c([2H])c(-c2c([2H])c([2H])c(-c3c([2H])c([2H])c([2H])c(-n4c5ccccc5c5cc(B(O)O)ccc54)c3[2H])c([2H])c2[2H])c([2H])c1[2H]] |
| 2-52 | 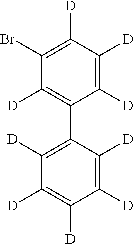 [SMILES: [2H]c1c([2H])c([2H])c(-c2c([2H])c([2H])c([2H])c(Br)c2[2H])c([2H])c1[2H]] | 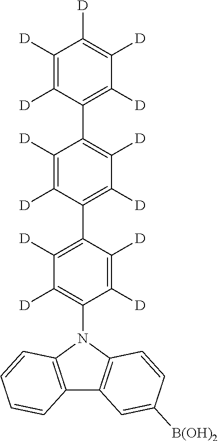 [SMILES: [2H]c1c([2H])c([2H])c(-c2c([2H])c([2H])c(-c3c([2H])c([2H])c(-n4c5ccccc5c5cc(B(O)O)ccc54)c([2H])c3[2H])c([2H])c2[2H])c([2H])c1[2H]] |
| 2-55 | 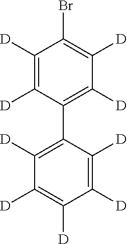 [SMILES: [2H]c1c([2H])c([2H])c(-c2c([2H])c([2H])c(Br)c([2H])c2[2H])c([2H])c1[2H]] | 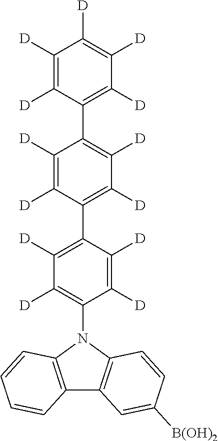 [SMILES: [2H]c1c([2H])c([2H])c(-c2c([2H])c([2H])c(-c3c([2H])c([2H])c(-n4c5ccccc5c5cc(B(O)O)ccc54)c([2H])c3[2H])c([2H])c2[2H])c([2H])c1[2H]] |
| 2-57 | 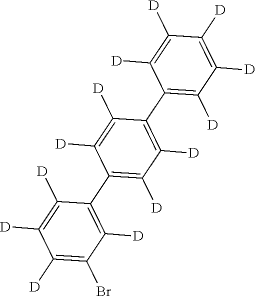 [SMILES: [2H]c1c([2H])c([2H])c(-c2c([2H])c([2H])c(-c3c([2H])c([2H])c([2H])c(Br)c3[2H])c([2H])c2[2H])c([2H])c1[2H]] | 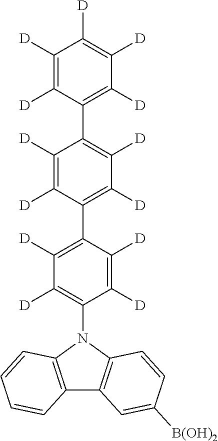 [SMILES: [2H]c1c([2H])c([2H])c(-c2c([2H])c([2H])c(-c3c([2H])c([2H])c(-n4c5ccccc5c5cc(B(O)O)ccc54)c([2H])c3[2H])c([2H])c2[2H])c([2H])c1[2H]] |
| 2-60 | 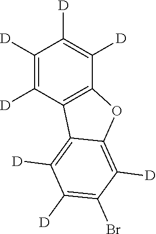 [SMILES: [2H]c1c([2H])c([2H])c2c(oc3c([2H])c(Br)c([2H])c([2H])c32)c1[2H]] | 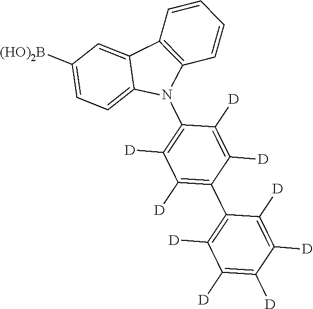 [SMILES: [2H]c1c([2H])c([2H])c(-c2c([2H])c([2H])c(-n3c4ccccc4c4cc(B(O)O)ccc43)c([2H])c2[2H])c([2H])c1[2H]] |

| Com- | |
|---|---|
| pound | Target Compound |
| No. | (Yield %) |
| 2-2 | 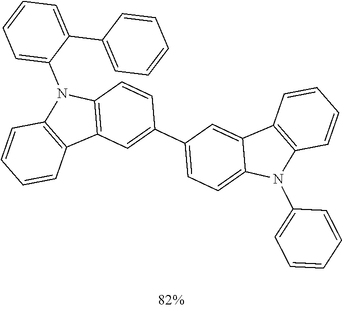 [SMILES: c1ccc(-c2ccccc2-n2c3ccccc3c3cc(-c4ccc5c(c4)c4ccccc4n5-c4ccccc4)ccc32)cc1] |
| 2-3 | 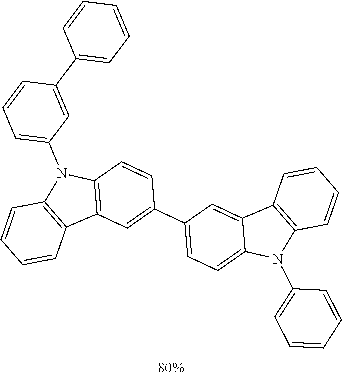 [SMILES: c1ccc(-c2cccc(-n3c4ccccc4c4cc(-c5ccc6c(c5)c5ccccc5n6-c5ccccc5)ccc43)c2)cc1] |
| 2-4 | 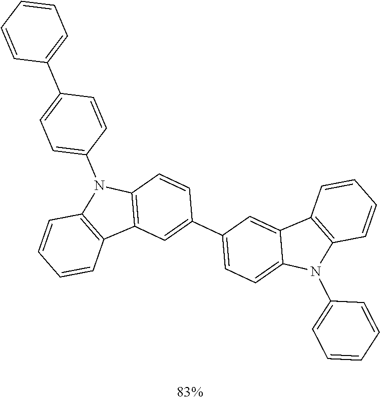 [SMILES: c1ccc(-c2ccc(-n3c4ccccc4c4cc(-c5ccc6c(c5)c5ccccc5n6-c5ccccc5)ccc43)cc2)cc1] |
| 2-5 | 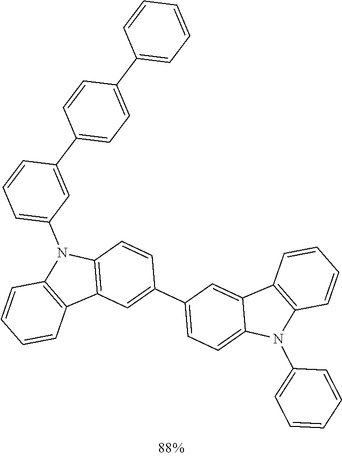 [SMILES: c1ccc(-c2ccc(-c3cccc(-n4c5ccccc5c5cc(-c6ccc7c(c6)c6ccccc6n7-c6ccccc6)ccc54)c3)cc2)cc1] |
| 2-6 | 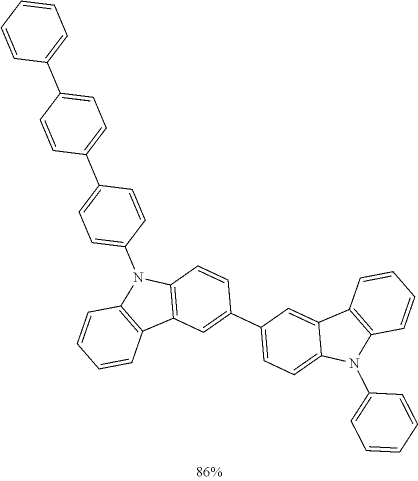 [SMILES: c1ccc(-c2ccc(-c3ccc(-n4c5ccccc5c5cc(-c6ccc7c(c6)c6ccccc6n7-c6ccccc6)ccc54)cc3)cc2)cc1] |
| 2-7 | 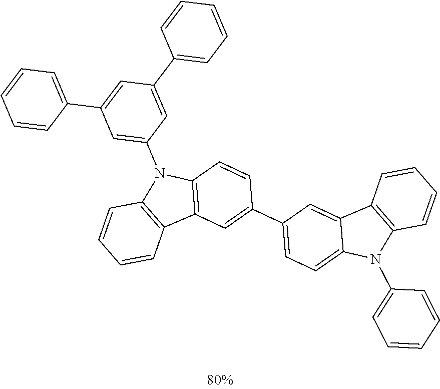 [SMILES: c1ccc(-c2cc(-c3ccccc3)cc(-n3c4ccccc4c4cc(-c5ccc6c(c5)c5ccccc5n6-c5ccccc5)ccc43)c2)cc1] |
| 2-11 | 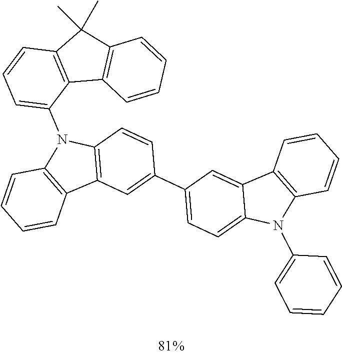 [SMILES: CC1(C)c2ccccc2-c2c(-n3c4ccccc4c4cc(-c5ccc6c(c5)c5ccccc5n6-c5ccccc5)ccc43)cccc21] |
| 2-16 | 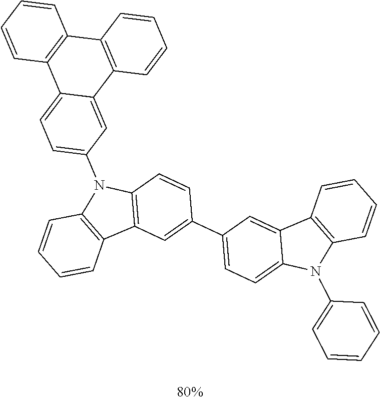 [SMILES: c1ccc(-n2c3ccccc3c3cc(-c4ccc5c(c4)c4ccccc4n5-c4ccc5c6ccccc6c6ccccc6c5c4)ccc32)cc1] |
| 2-19 | 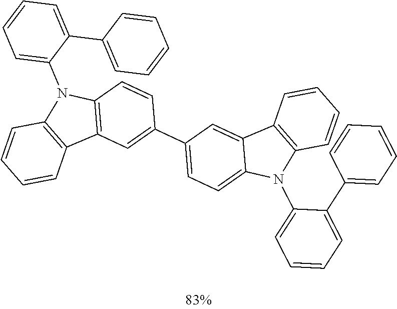 [SMILES: c1ccc(-c2ccccc2-n2c3ccccc3c3cc(-c4ccc5c(c4)c4ccccc4n5-c4ccccc4-c4ccccc4)ccc32)cc1] |
| 2-20 | 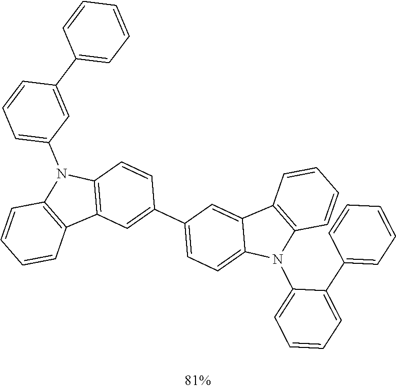 [SMILES: c1ccc(-c2cccc(-n3c4ccccc4c4cc(-c5ccc6c(c5)c5ccccc5n6-c5ccccc5-c5ccccc5)ccc43)c2)cc1] |
| 2-21 | 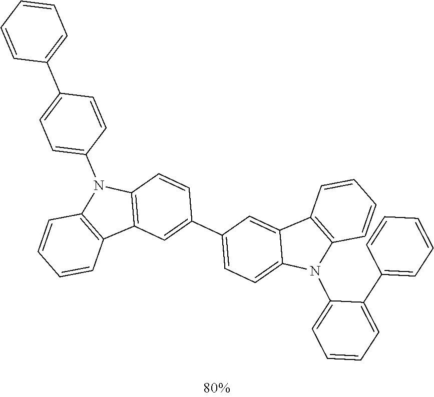 [SMILES: c1ccc(-c2ccc(-n3c4ccccc4c4cc(-c5ccc6c(c5)c5ccccc5n6-c5ccccc5-c5ccccc5)ccc43)cc2)cc1] |
| 2-22 | 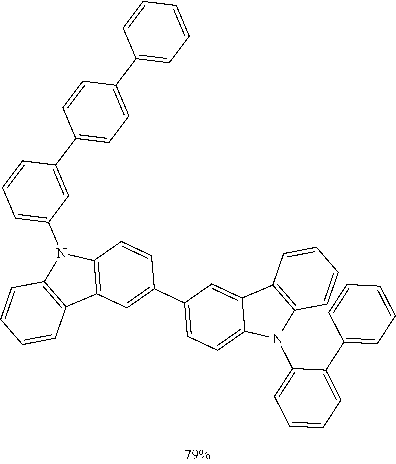 [SMILES: c1ccc(-c2ccc(-c3cccc(-n4c5ccccc5c5cc(-c6ccc7c(c6)c6ccccc6n7-c6ccccc6-c6ccccc6)ccc54)c3)cc2)cc1] |
| 2-23 | 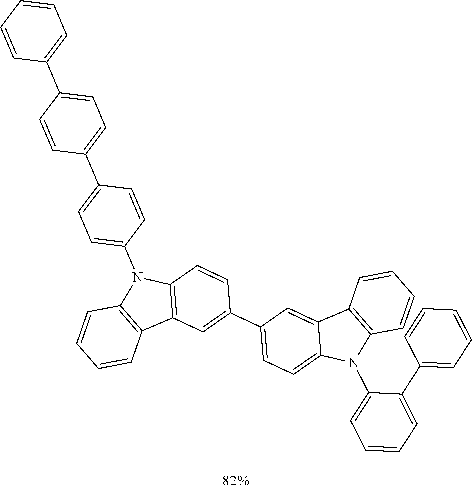 [SMILES: c1ccc(-c2ccc(-c3ccc(-n4c5ccccc5c5cc(-c6ccc7c(c6)c6ccccc6n7-c6ccccc6-c6ccccc6)ccc54)cc3)cc2)cc1] |
| 2-26 | 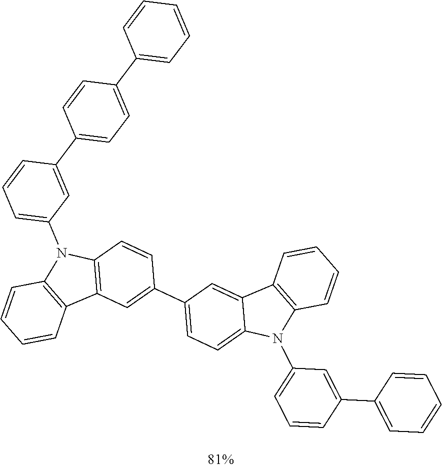 [SMILES: c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1] |
| 2-27 | 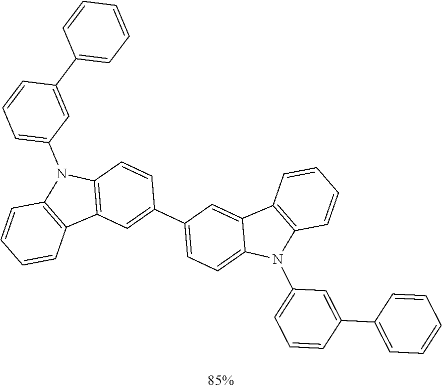 [SMILES: c1ccc(-c2cccc(-n3c4ccccc4c4cc(-c5ccc6c(c5)c5ccccc5n6-c5cccc(-c6ccccc6)c5)ccc43)c2)cc1] |
| 2-28 | 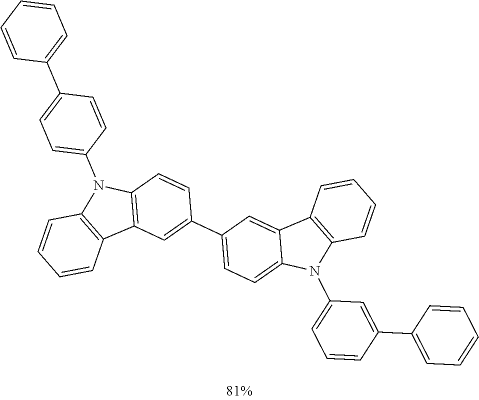 [SMILES: c1ccc(-c2ccc(-n3c4ccccc4c4cc(-c5ccc6c(c5)c5ccccc5n6-c5cccc(-c6ccccc6)c5)ccc43)cc2)cc1] |
| 2-29 | 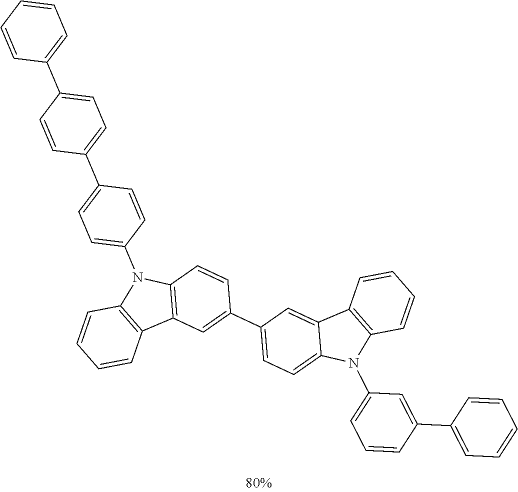 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1] |
| 2-30 | 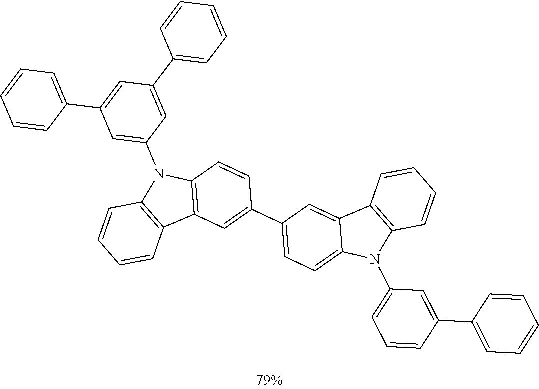 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1] |
| 2-32 | 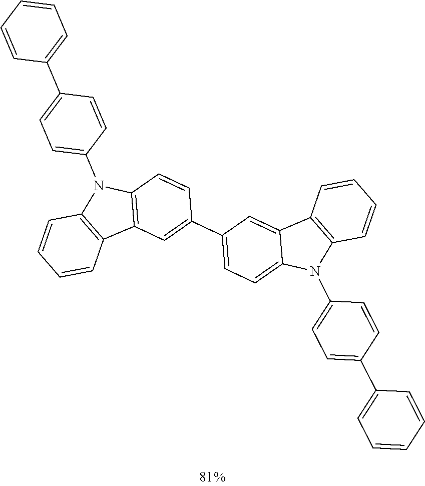 [SMILES: c1ccc(-c2ccc(-n3c4ccccc4c4cc(-c5ccc6c(c5)c5ccccc5n6-c5ccc(-c6ccccc6)cc5)ccc43)cc2)cc1] |
| 2-33 | 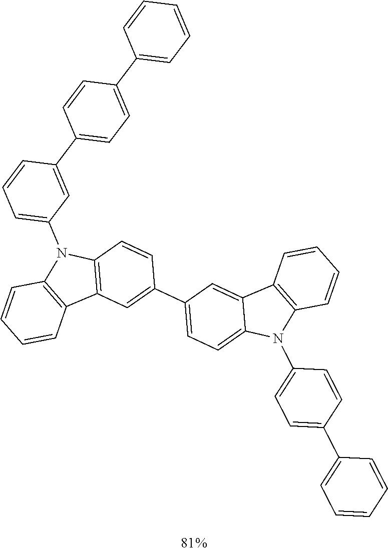 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1] |
| 2-34 | 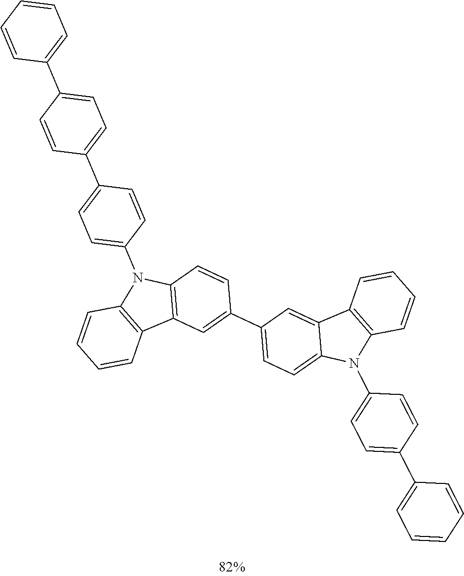 [SMILES: c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1] |
| 2-38 | 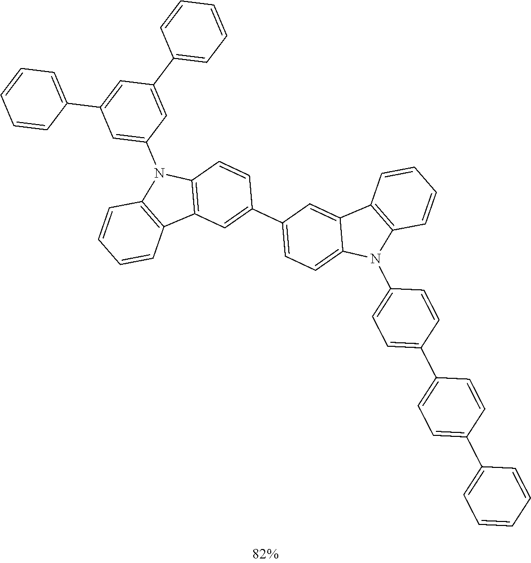 [SMILES: c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1] |
| 2-40 | 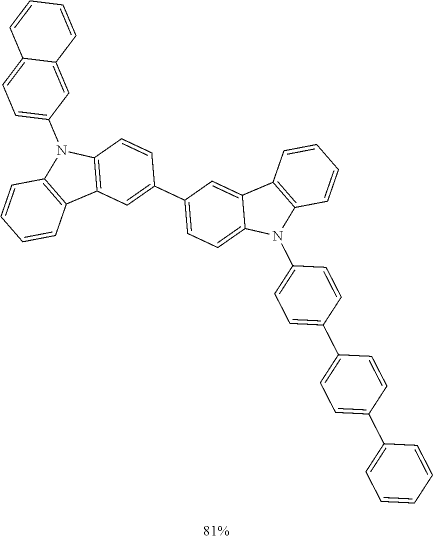 [SMILES: c1ccc(-c2ccc(-c3ccc(-n4c5ccccc5c5cc(-c6ccc7c(c6)c6ccccc6n7-c6ccc7ccccc7c6)ccc54)cc3)cc2)cc1] |
| 2-41 | 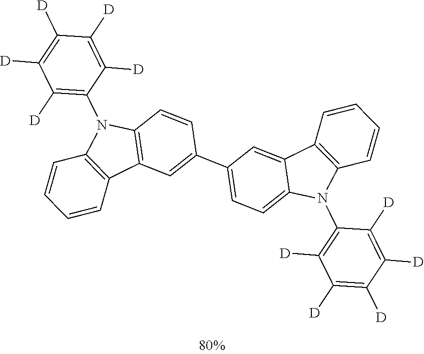 [SMILES: [2H]c1c([2H])c([2H])c(-n2c3ccccc3c3cc(-c4ccc5c(c4)c4ccccc4n5-c4c([2H])c([2H])c([2H])c([2H])c4[2H])ccc32)c([2H])c1[2H]] |
| 2-42 | 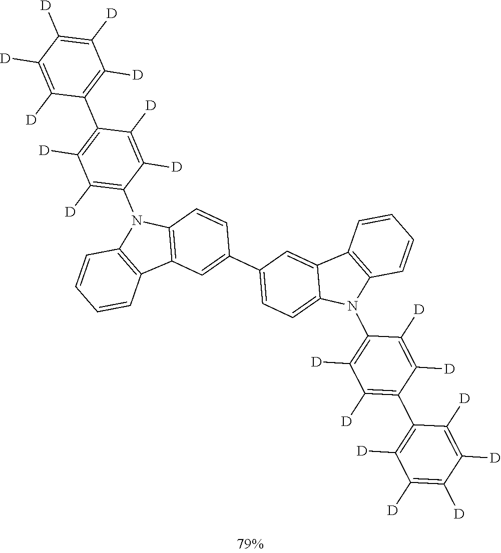 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c([2H])c6[2H])c([2H])c5[2H])ccc43)c([2H])c2[2H])c([2H])c1[2H]] |
| 2-43 | 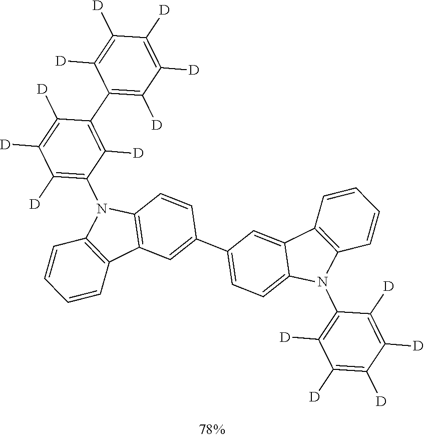 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c([2H])c5[2H])ccc43)c2[2H])c([2H])c1[2H]] |
| 2-45 | 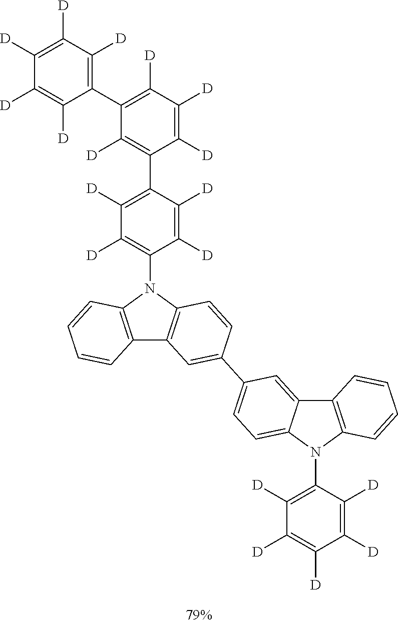 [SMILES: [2H]c1c([2H])c([2H])c(-c2c([2H])c([2H])c([2H])c(-c3c([2H])c([2H])c(-n4c5ccccc5c5cc(-c6ccc7c(c6)c6ccccc6n7-c6c([2H])c([2H])c([2H])c([2H])c6[2H])ccc54)c([2H])c3[2H])c2[2H])c([2H])c1[2H]] |
| 2-46 | 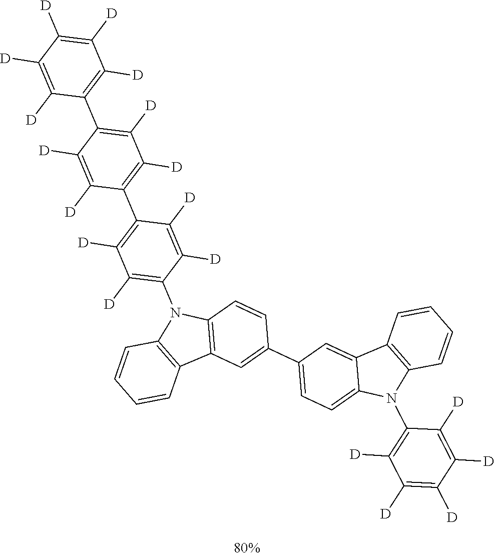 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c([2H])c([2H])c6[2H])ccc54)c([2H])c3[2H])c([2H])c2[2H])c([2H])c1[2H]] |
| 2-48 | 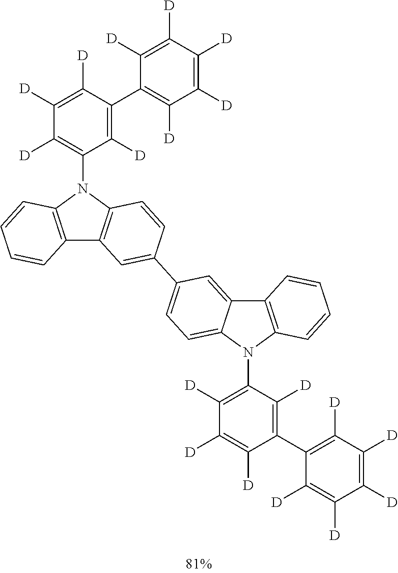 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c2[2H])c([2H])c1[2H]] |
| 2-49 | 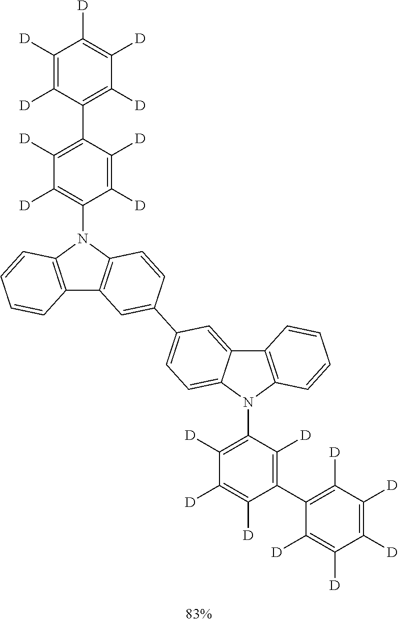 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c([2H])c2[2H])c([2H])c1[2H]] |
| 2-50 | 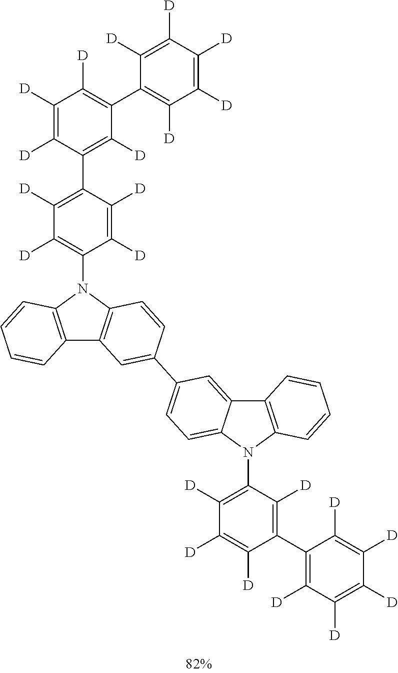 [SMILES: [2H]c1c([2H])c([2H])c(-c2c([2H])c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c([2H])c3[2H])c2[2H])c([2H])c1[2H]] |
| 2-51 | 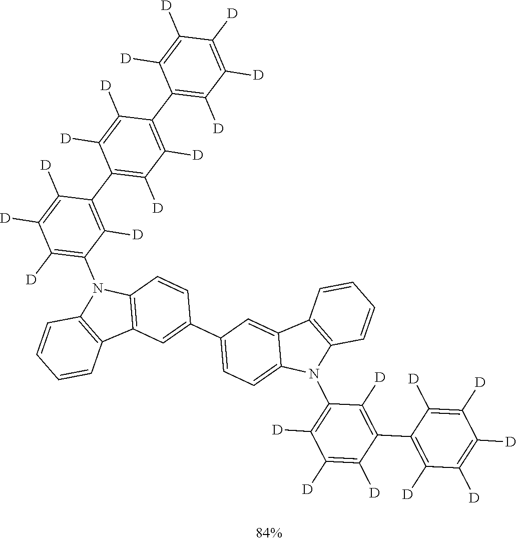 [SMILES: [2H]c1c([2H])c([2H])c(-c2c([2H])c([2H])c(-c3c([2H])c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c3[2H])c([2H])c2[2H])c([2H])c1[2H]] |
| 2-52 | 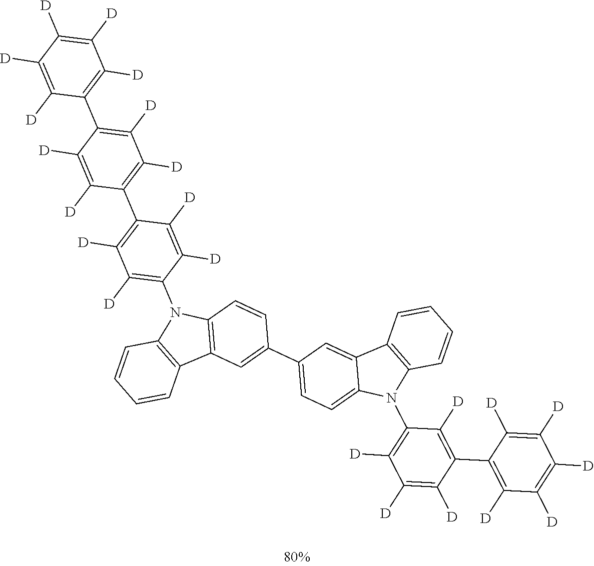 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c([2H])c3[2H])c([2H])c2[2H])c([2H])c1[2H]] |
| 2-55 | 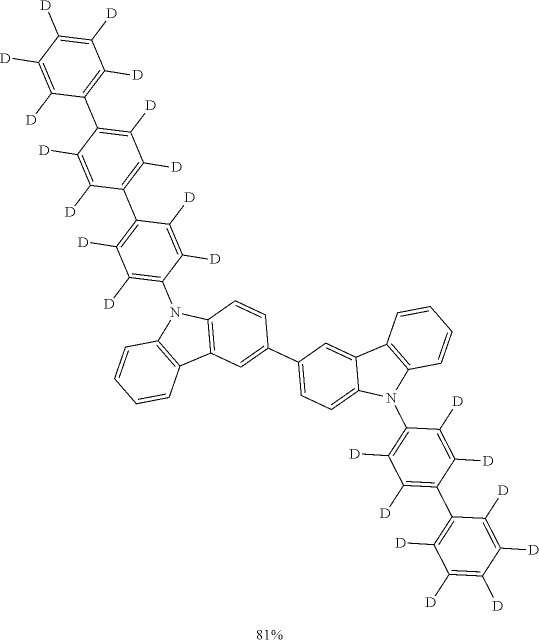 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c(-c7c([2H])c([2H])c([2H])c([2H])c7[2H])c([2H])c6[2H])ccc54)c([2H])c3[2H])c([2H])c2[2H])c([2H])c1[2H]] |
| 2-57 | 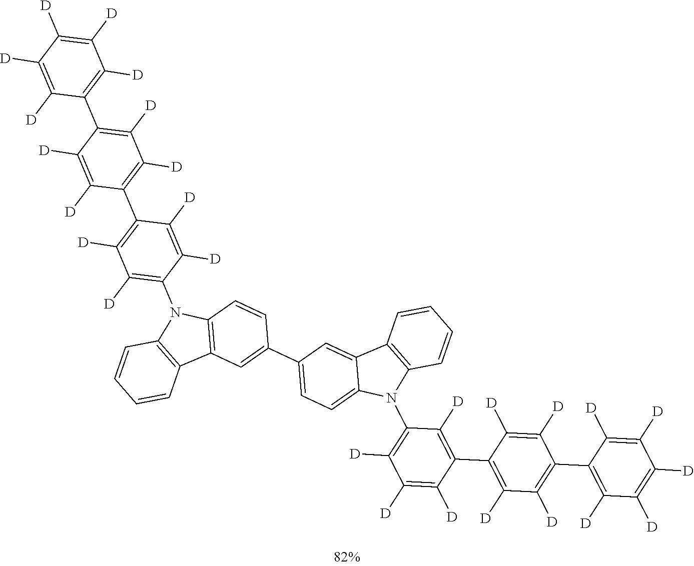 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c(-c8c([2H])c([2H])c([2H])c([2H])c8[2H])c([2H])c7[2H])c6[2H])ccc54)c([2H])c3[2H])c([2H])c2[2H])c([2H])c1[2H]] |
| 2-60 | 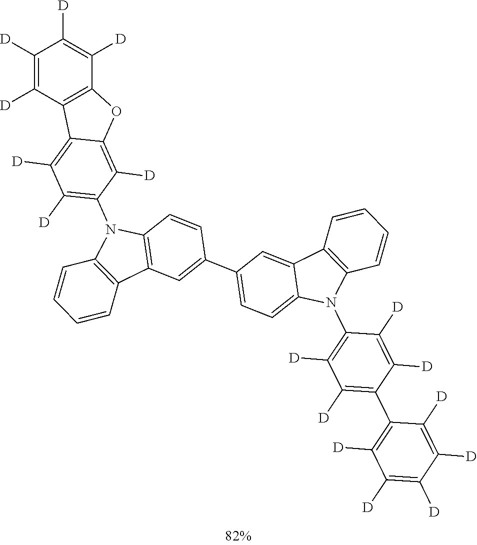 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c6c(oc7c([2H])c([2H])c([2H])c([2H])c76)c5[2H])ccc43)c([2H])c2[2H])c([2H])c1[2H]] |

### <Preparation Example 5> Preparation of Compound 2-61

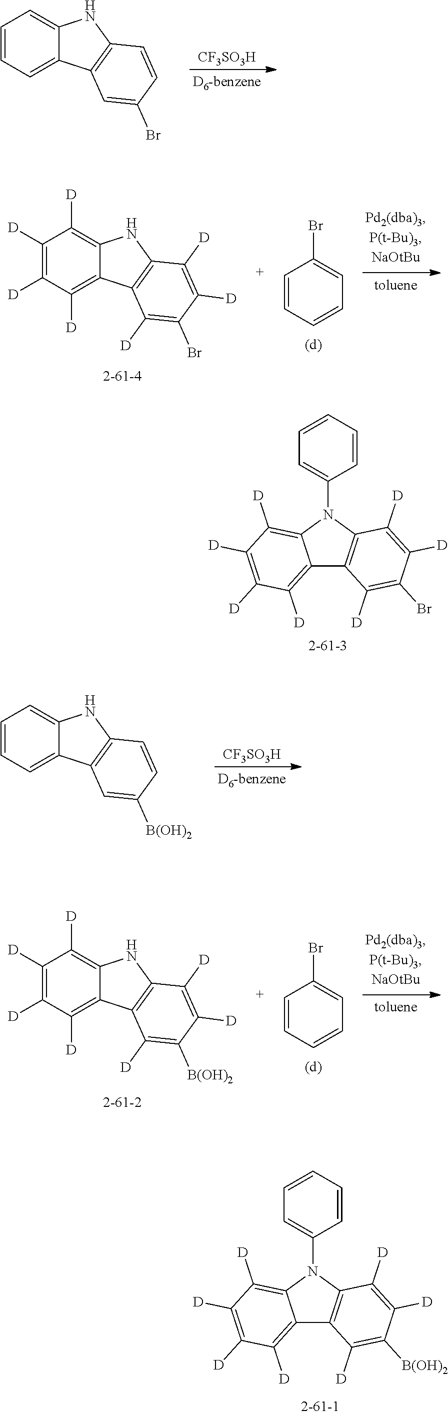

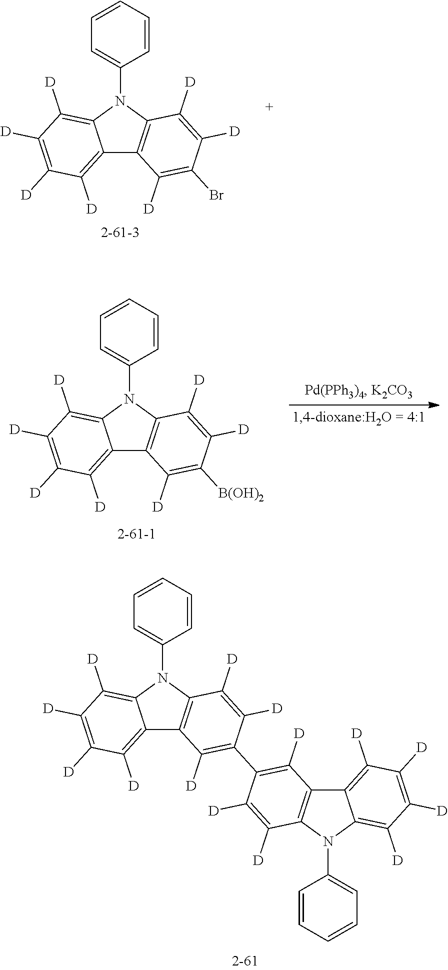 [SMILES: [2H]c1c([2H])c([2H])c2c(c1[2H])c1c([2H])c(Br)c([2H])c([2H])c1n2-c1ccccc1 ; [2H]c1c([2H])c([2H])c2c(c1[2H])c1c([2H])c(B(O)O)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1]

### 1) Preparation of Compound 2-61-4

10 g (40.23 mmol) of 3-bromo-9H-carbazole, 1,000 mL of D₆-benzene, and 170 g (1,075 mmol) of triflic acid (CF₃SO₃H) were put into a reaction flask, and the resulting mixture was stirred at 50° C.

After the reaction was completed, the resulting product was neutralized with D₂O and then extracted with an aqueous sodium carbonate (Na₂CO₃) solution and dichloromethane (DCM) at room temperature, the organic layer was dried over anhydrous magnesium sulfate (MgSO₄), and then the solvent was removed using a rotary evaporator. The reaction product was purified by column chromatography (dichloromethane:hexane=1:2) and recrystallized with methanol to obtain 10 g (yield 98%) of Target Compound 2-61-4.

### 2) Preparation of Compound 2-61-3

After 10 g (39.5 mmol) of Compound 2-61-4, 12.4 g (79 mmol) of 2-bromobenzene (c), 1.81 g (1.98 mmol) of tris(dibenzylideneacetone)dipalladium (Pd₂(dba)₃), 1.93 mL (7.9 mmol) of tri-tert-butylphosphine (P(t-Bu)₃), and 11.4 g (118.51 mmol) of sodium tert-butoxide (NatOBu) were put into a reaction flask, 100 mL of toluene was added thereto, and the resulting mixture was heated at 135° C. for 15 hours. When the reaction was terminated, the resulting product was extracted with methylene chloride (MC) and water, and then purified by column chromatography to obtain 11 g (yield 84%) of Compound 2-61-3.

### 3) Preparation of Compound 2-61-2

10 g (47.3 mmol) of 9H-carbazol-3-ylboronic acid, 1,000 mL of D₆-benzene, and 170 g (1,075 mmol) of triflic acid (CF₃SO₃H) were put into a reaction flask, and the resulting mixture was stirred at 50° C.

After the reaction was completed, the resulting product was neutralized with D₂O and then extracted with an aqueous sodium carbonate (Na₂CO₃) solution and dichloromethane (DCM) at room temperature, the organic layer was dried over anhydrous magnesium sulfate (MgSO₄), and then the solvent was removed using a rotary evaporator. The reaction product was purified by column chromatography (dichloromethane:hexane=1:2) and recrystallized with methanol to obtain 9 g (yield 87%) of Target Compound 2-61-2.

### 4) Preparation of Compound 2-61-1

After 9 g (41.3 mmol) of Compound 2-61-2, 12.9 g (82.5 mmol) of bromobenzene (d), 1.89 g (2.06 mmol) of tris(dibenzylideneacetone)dipalladium (Pd₂(dba)₃), 2 mL (8.25 mmol) of tri-tert-butylphosphine (P(t-Bu)₃), and 7.93 g (82.574 mmol) of sodium tert-butoxide (NatOBu) were put into a reaction flask, 100 mL of toluene was added thereto, and the resulting mixture was heated at 135° C. for 10 hours. When the reaction was terminated, the resulting product was extracted with methylene chloride (MC) and water, and then purified by column chromatography to obtain 10 g (yield 82%) of Compound 2-61-1.

### 5) Preparation of Compound 2-61

After 10 g (30.37 mmol) of Compound 2-61-3, 17.87 g (60.75 mmol) of Compound 2-61-1, 1.39 g (1.52 mmol) of tetrakis(triphenylphosphine)palladium(0) (Pd(PPh₃)₄), and 12.59 g (91.13 mmol) of potassium carbonate (K₂CO₃) were put into a reaction flask, 140 mL of 1,4-dioxane and 35 mL of distilled water were added thereto, and the resulting mixture was stirred at 120° C. for 4 hours.

Thereafter, a solid produced by lowering the temperature to room temperature was washed with distilled water and methanol to obtain 13 g (yield 85%) of Compound 2-61.

### <Preparation Example 6> Preparation of Compounds 2-62, 2-63, 2-64, 2-65, 2-66, 2-68, 2-69, 2-70, 2-74, 2-75, 2-81, 2-82, 2-83, 2-85, 2-86, 2-87, 2-88, 2-89, 2-90, 2-92, 2-100 and 2-102

Compounds 2-62, 2-63, 2-64, 2-65, 2-66, 2-68, 2-69, 2-70, 2-74, 2-75, 2-81, 2-82, 2-83, 2-85, 2-86, 2-87, 2-88, 2-89, 2-90, 2-92, 2-100 and 2-102 were synthesized in the same manner as in the preparation of Preparation Example 5, except that Compound c and Compound d in the following Table 5 were used instead of bromobenzene (c) and bromobenzene (d), respectively, in Preparation Example 5.

TABLE 5

| Compound | | |
|---|---|---|
| No. | Compound c | Compound d |
| 2-62 | 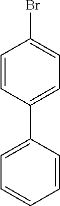 [SMILES: Brc1ccc(-c2ccccc2)cc1] | 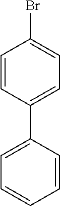 [SMILES: Brc1ccc(-c2ccccc2)cc1] |
| 2-63 | 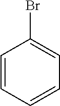 | 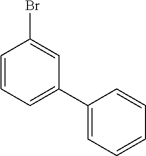 [SMILES: Brc1cccc(-c2ccccc2)c1] |
| 2-64 | 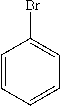 | 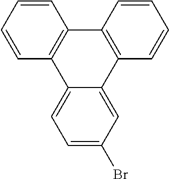 [SMILES: Brc1ccc2c3ccccc3c3ccccc3c2c1] |
| 2-65 | 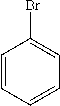 | 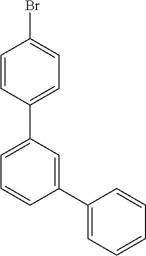 [SMILES: Brc1ccc(-c2cccc(-c3ccccc3)c2)cc1] |
| 2-66 | 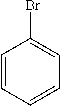 | 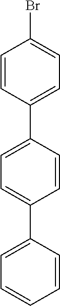 [SMILES: Brc1ccc(-c2ccc(-c3ccccc3)cc2)cc1] |
| 2-68 | 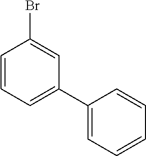 [SMILES: Brc1cccc(-c2ccccc2)c1] | 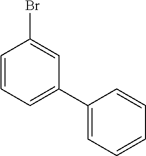 [SMILES: Brc1cccc(-c2ccccc2)c1] |
| 2-69 | 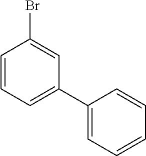 [SMILES: Brc1cccc(-c2ccccc2)c1] | 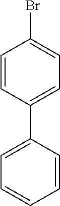 [SMILES: Brc1ccc(-c2ccccc2)cc1] |
| 2-70 | 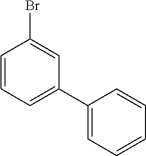 [SMILES: Brc1cccc(-c2ccccc2)c1] | 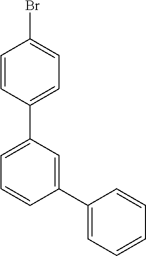 [SMILES: Brc1ccc(-c2cccc(-c3ccccc3)c2)cc1] |
| 2-74 | 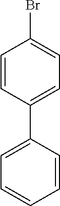 [SMILES: Brc1ccc(-c2ccccc2)cc1] | 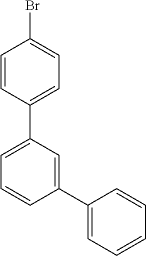 [SMILES: Brc1ccc(-c2cccc(-c3ccccc3)c2)cc1] |
| 2-75 | 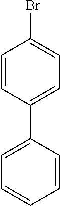 [SMILES: Brc1ccc(-c2ccccc2)cc1] | 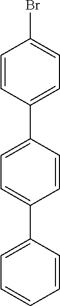 [SMILES: Brc1ccc(-c2ccc(-c3ccccc3)cc2)cc1] |
| 2-81 | 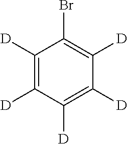 [SMILES: [2H]c1c([2H])c([2H])c(Br)c([2H])c1[2H]] | 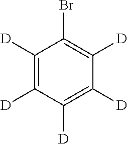 [SMILES: [2H]c1c([2H])c([2H])c(Br)c([2H])c1[2H]] |
| 2-82 | 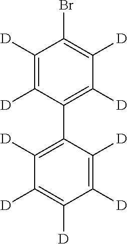 [SMILES: [2H]c1c([2H])c([2H])c(-c2c([2H])c([2H])c(Br)c([2H])c2[2H])c([2H])c1[2H]] | 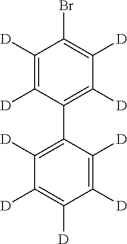 [SMILES: [2H]c1c([2H])c([2H])c(-c2c([2H])c([2H])c(Br)c([2H])c2[2H])c([2H])c1[2H]] |
| 2-83 | 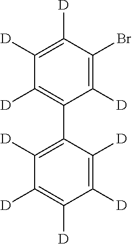 [SMILES: [2H]c1c([2H])c([2H])c(-c2c([2H])c([2H])c([2H])c(Br)c2[2H])c([2H])c1[2H]] | 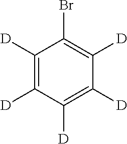 [SMILES: [2H]c1c([2H])c([2H])c(Br)c([2H])c1[2H]] |
| 2-85 | 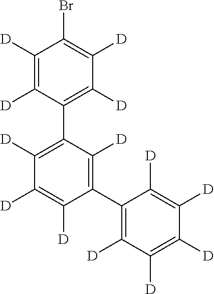 [SMILES: [2H]c1c([2H])c([2H])c(-c2c([2H])c([2H])c([2H])c(-c3c([2H])c([2H])c(Br)c([2H])c3[2H])c2[2H])c([2H])c1[2H]] | 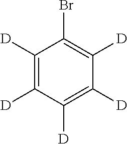 [SMILES: [2H]c1c([2H])c([2H])c(Br)c([2H])c1[2H]] |
| 2-86 | 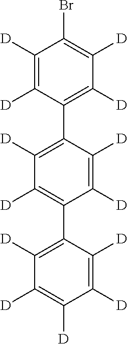 [SMILES: [2H]c1c([2H])c([2H])c(-c2c([2H])c([2H])c(-c3c([2H])c([2H])c(Br)c([2H])c3[2H])c([2H])c2[2H])c([2H])c1[2H]] | 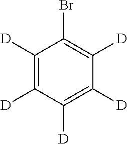 [SMILES: [2H]c1c([2H])c([2H])c(Br)c([2H])c1[2H]] |
| 2-87 | 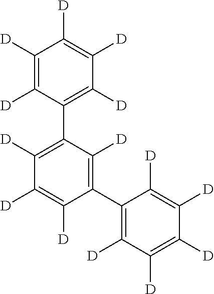 [SMILES: [2H]c1c([2H])c([2H])c(-c2c([2H])c([2H])c([2H])c(-c3c([2H])c([2H])c([2H])c([2H])c3[2H])c2[2H])c([2H])c1[2H]] | 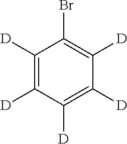 [SMILES: [2H]c1c([2H])c([2H])c(Br)c([2H])c1[2H]] |
| 2-88 | 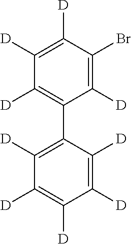 [SMILES: [2H]c1c([2H])c([2H])c(-c2c([2H])c([2H])c([2H])c(Br)c2[2H])c([2H])c1[2H]] | 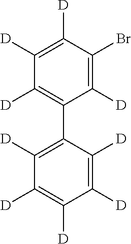 [SMILES: [2H]c1c([2H])c([2H])c(-c2c([2H])c([2H])c([2H])c(Br)c2[2H])c([2H])c1[2H]] |
| 2-89 | 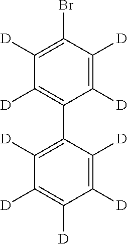 [SMILES: [2H]c1c([2H])c([2H])c(-c2c([2H])c([2H])c(Br)c([2H])c2[2H])c([2H])c1[2H]] | 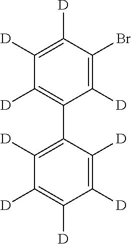 [SMILES: [2H]c1c([2H])c([2H])c(-c2c([2H])c([2H])c([2H])c(Br)c2[2H])c([2H])c1[2H]] |
| 2-90 | 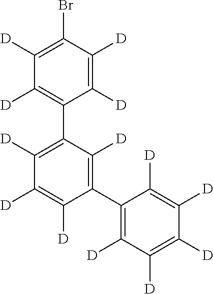 [SMILES: [2H]c1c([2H])c([2H])c(-c2c([2H])c([2H])c([2H])c(-c3c([2H])c([2H])c(Br)c([2H])c3[2H])c2[2H])c([2H])c1[2H]] | 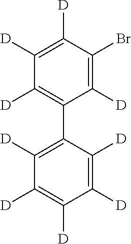 [SMILES: [2H]c1c([2H])c([2H])c(-c2c([2H])c([2H])c([2H])c(Br)c2[2H])c([2H])c1[2H]] |
| 2-92 | 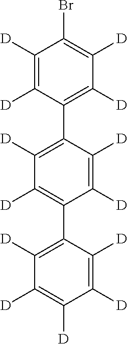 [SMILES: [2H]c1c([2H])c([2H])c(-c2c([2H])c([2H])c(-c3c([2H])c([2H])c(Br)c([2H])c3[2H])c([2H])c2[2H])c([2H])c1[2H]] | 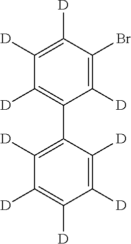 [SMILES: [2H]c1c([2H])c([2H])c(-c2c([2H])c([2H])c([2H])c(Br)c2[2H])c([2H])c1[2H]] |
| 2-100 | 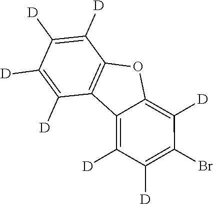 [SMILES: [2H]c1c([2H])c([2H])c2c(oc3c([2H])c(Br)c([2H])c([2H])c32)c1[2H]] | 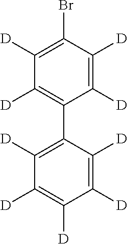 [SMILES: [2H]c1c([2H])c([2H])c(-c2c([2H])c([2H])c(Br)c([2H])c2[2H])c([2H])c1[2H]] |
| 2-102 | 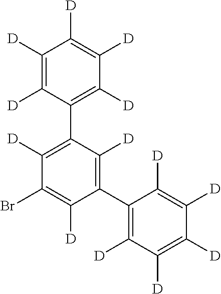 [SMILES: [2H]c1c([2H])c([2H])c(-c2c([2H])c(Br)c([2H])c(-c3c([2H])c([2H])c([2H])c([2H])c3[2H])c2[2H])c([2H])c1[2H]] | 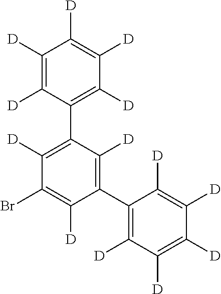 [SMILES: [2H]c1c([2H])c([2H])c(-c2c([2H])c(Br)c([2H])c(-c3c([2H])c([2H])c([2H])c([2H])c3[2H])c2[2H])c([2H])c1[2H]] |

| Compound | Target Compound |
|---|---|
| No. | (Yield %) |
| 2-62 | 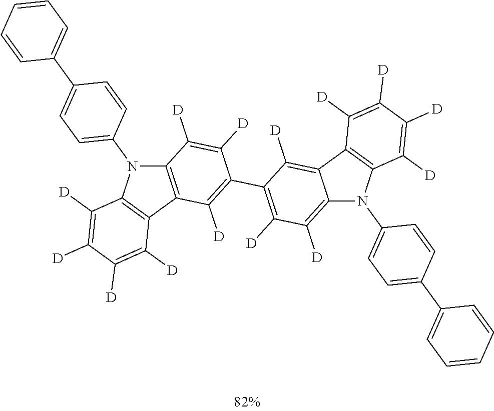 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1] |
| 2-63 | 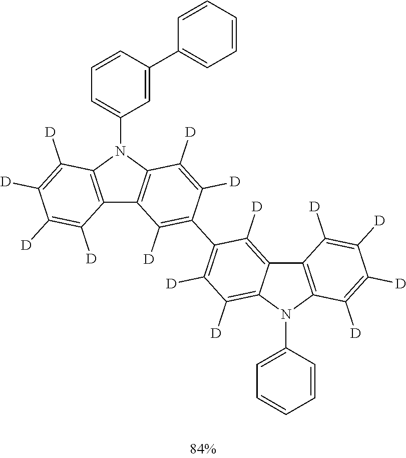 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccccc1] |
| 2-64 | 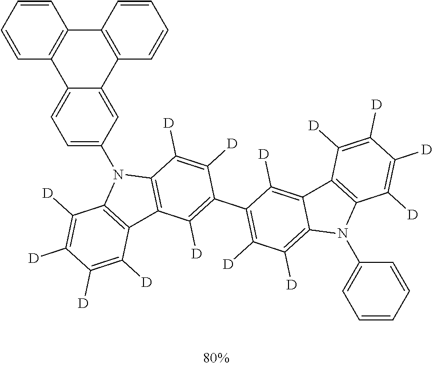 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c5ccccc5c5ccccc5c4c3)c([2H])c([2H])c1n2-c1ccccc1] |
| 2-65 | 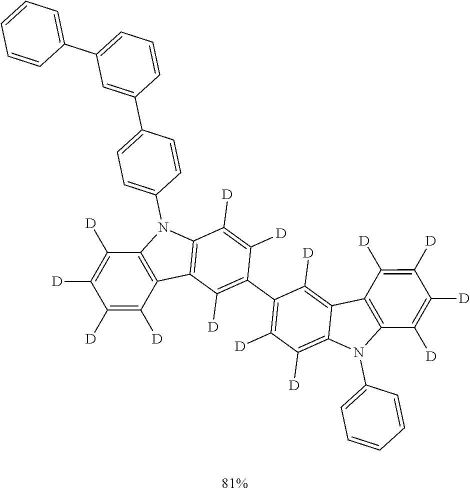 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4cccc(-c5ccccc5)c4)cc3)c([2H])c([2H])c1n2-c1ccccc1] |
| 2-66 | 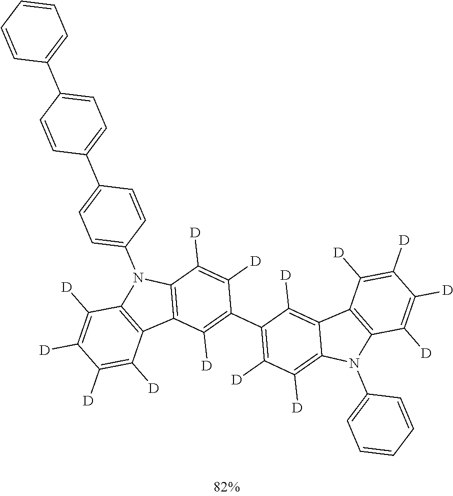 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccc(-c5ccccc5)cc4)cc3)c([2H])c([2H])c1n2-c1ccccc1] |
| 2-68 | 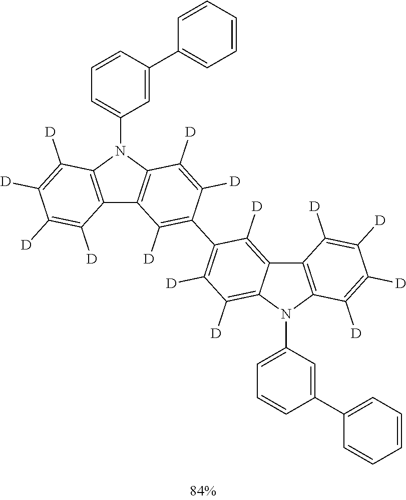 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1cccc(-c2ccccc2)c1] |
| 2-69 | 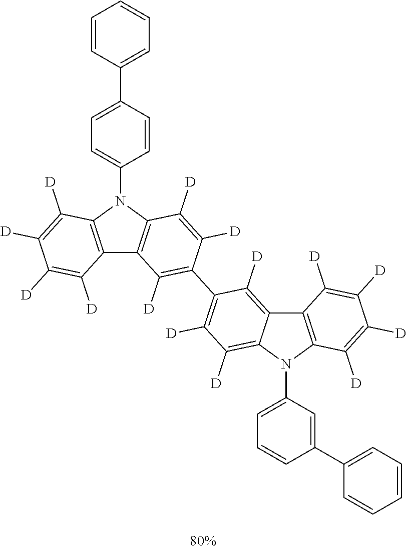 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccc(-c2ccccc2)cc1] |
| 2-70 | 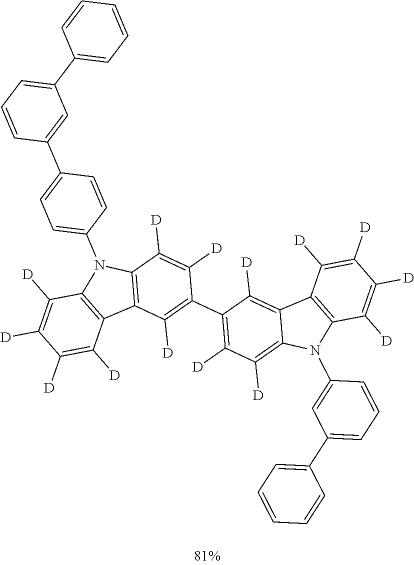 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccc(-c2cccc(-c3ccccc3)c2)cc1] |
| 2-74 | 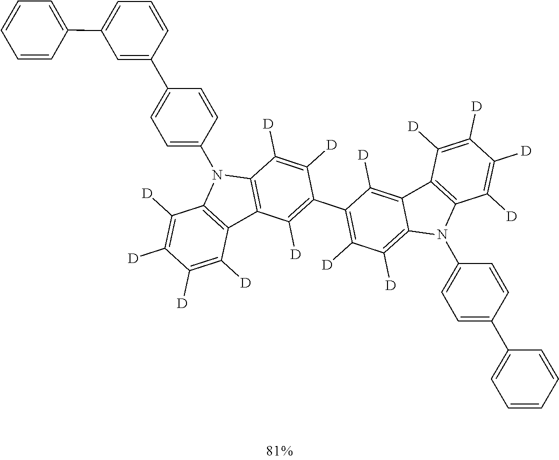 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4cccc(-c5ccccc5)c4)cc3)c([2H])c([2H])c1n2-c1ccc(-c2ccccc2)cc1] |
| 2-75 | 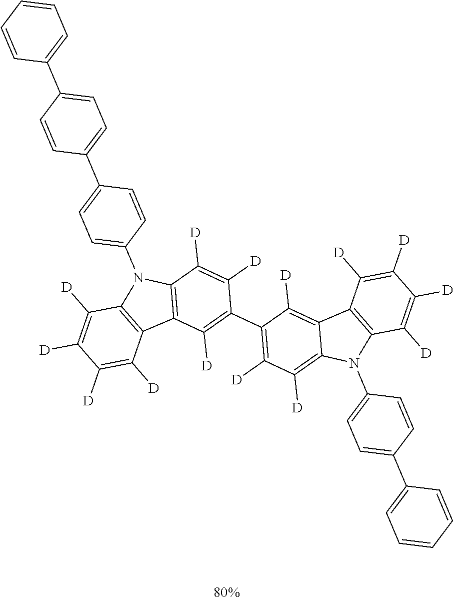 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccc(-c5ccccc5)cc4)cc3)c([2H])c([2H])c1n2-c1ccc(-c2ccccc2)cc1] |
| 2-81 | 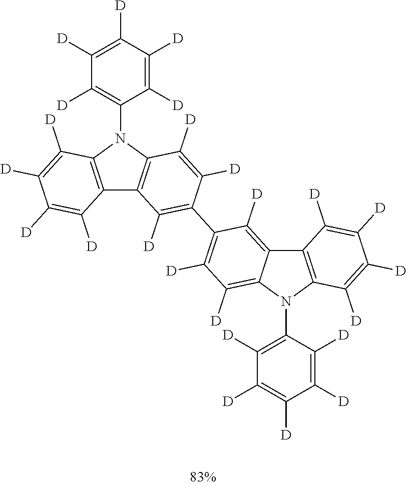 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H]] |
| 2-82 | 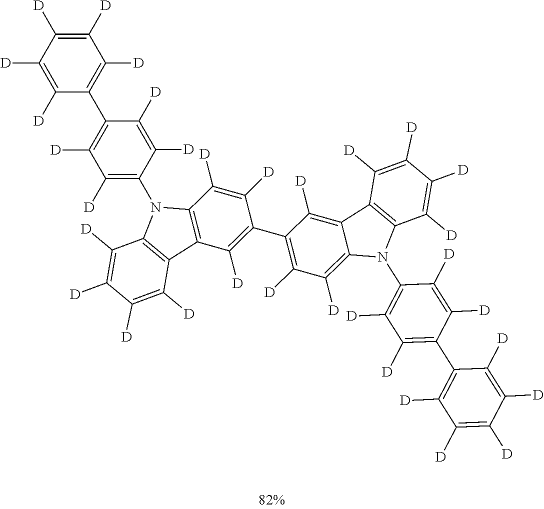 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c([2H])c1[2H]] |
| 2-83 | 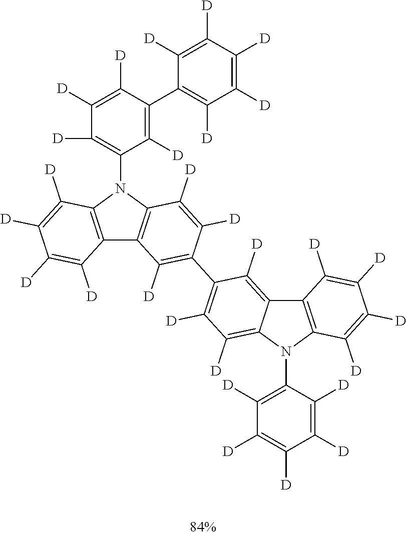 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H]] |
| 2-85 | 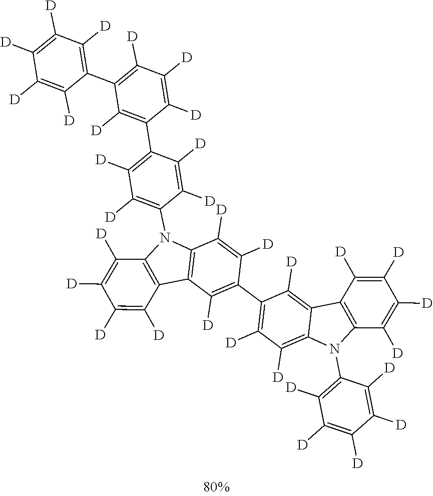 [SMILES: [2H]c1c([2H])c([2H])c(-c2c([2H])c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c([2H])c6[2H])c([2H])c([2H])c54)c([2H])c3[2H])c2[2H])c([2H])c1[2H]] |
| 2-86 | 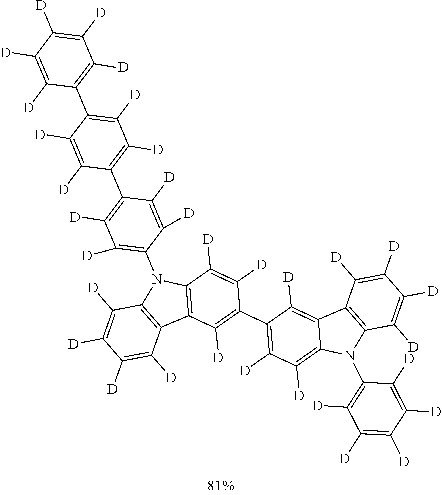 [SMILES: [2H]c1c([2H])c([2H])c(-c2c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c([2H])c6[2H])c([2H])c([2H])c54)c([2H])c3[2H])c([2H])c2[2H])c([2H])c1[2H]] |
| 2-87 | 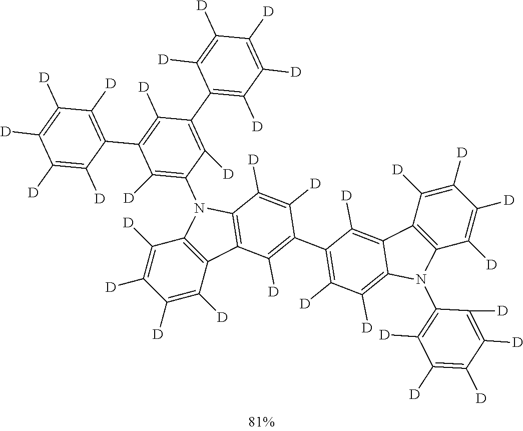 [SMILES: [2H]c1c([2H])c([2H])c(-c2c([2H])c(-c3c([2H])c([2H])c([2H])c([2H])c3[2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H]] |
| 2-88 | 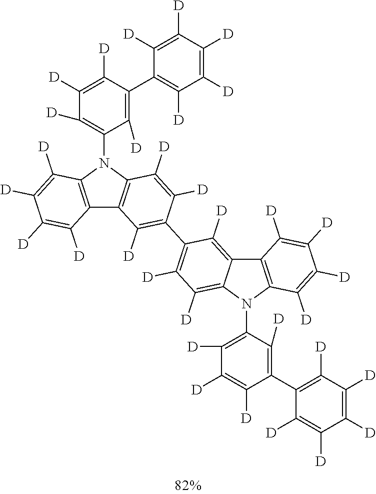 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c2[2H])c([2H])c1[2H]] |
| 2-89 | 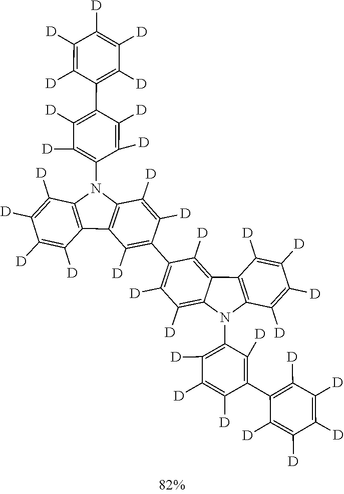 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c([2H])c2[2H])c([2H])c1[2H]] |
| 2-90 | 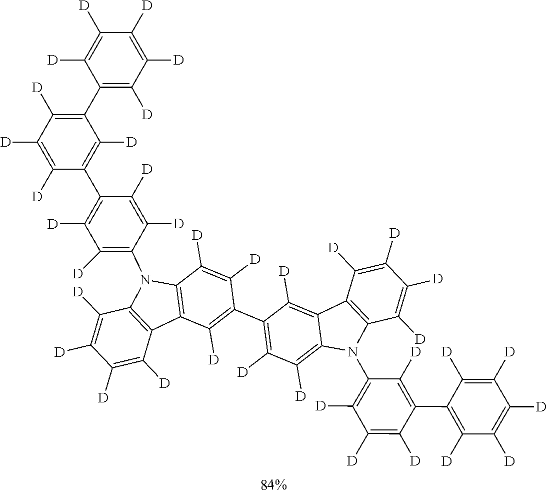 [SMILES: [2H]c1c([2H])c([2H])c(-c2c([2H])c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c(-c7c([2H])c([2H])c([2H])c([2H])c7[2H])c6[2H])c([2H])c([2H])c54)c([2H])c3[2H])c2[2H])c([2H])c1[2H]] |
| 2-92 | 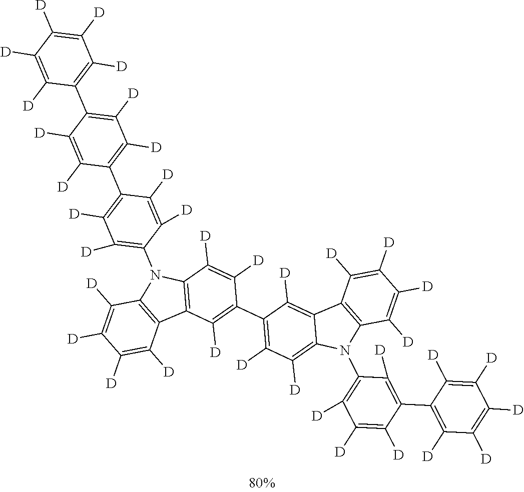 [SMILES: [2H]c1c([2H])c([2H])c(-c2c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c(-c7c([2H])c([2H])c([2H])c([2H])c7[2H])c6[2H])c([2H])c([2H])c54)c([2H])c3[2H])c([2H])c2[2H])c([2H])c1[2H]] |
| 2-100 | 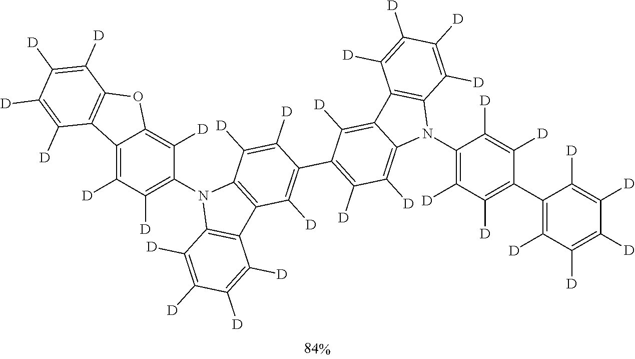 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6c(oc7c([2H])c([2H])c([2H])c([2H])c76)c5[2H])c([2H])c([2H])c43)c([2H])c2[2H])c([2H])c1[2H]] |
| 2-102 | 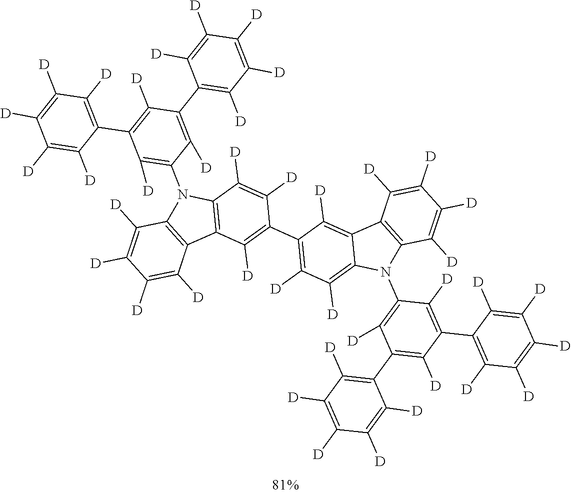 [SMILES: [2H]c1c([2H])c([2H])c(-c2c([2H])c(-c3c([2H])c([2H])c([2H])c([2H])c3[2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c(-c6c([2H])c([2H])c([2H])c([2H])c6[2H])c([2H])c(-c6c([2H])c([2H])c([2H])c([2H])c6[2H])c5[2H])c([2H])c([2H])c43)c2[2H])c([2H])c1[2H]] |

### <Preparation Example 7> Preparation of Compound 2-82

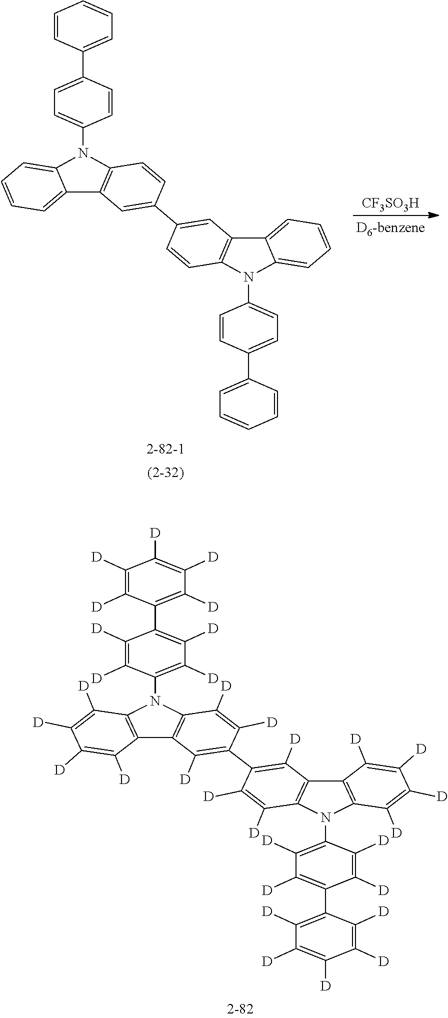 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; "CF3SO3H" ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c([2H])c1[2H]]

10 g (15.7 mmol) of Compound 2-82-1 (Compound 2-32), 1,000 mL of D₆-benzene, and 170 g (1,075 mmol) of triflic acid (CF₃SO₃H) were put into a reaction flask, and the resulting mixture was stirred at 50° C.

After the reaction was completed, the resulting product was neutralized with D₂O and then extracted with an aqueous sodium carbonate (Na₂CO₃) solution and dichloromethane (DCM) at room temperature, the organic layer was dried over anhydrous magnesium sulfate (MgSO₄), and then the solvent was removed using a rotary evaporator. The reaction product was purified by column chromatography (dichloromethane:hexane=1:2) and recrystallized with methanol to obtain 10.0 g (yield 95%) of Target Compound 2-82.

### <Preparation Example 8> Preparation of Compound 3-1

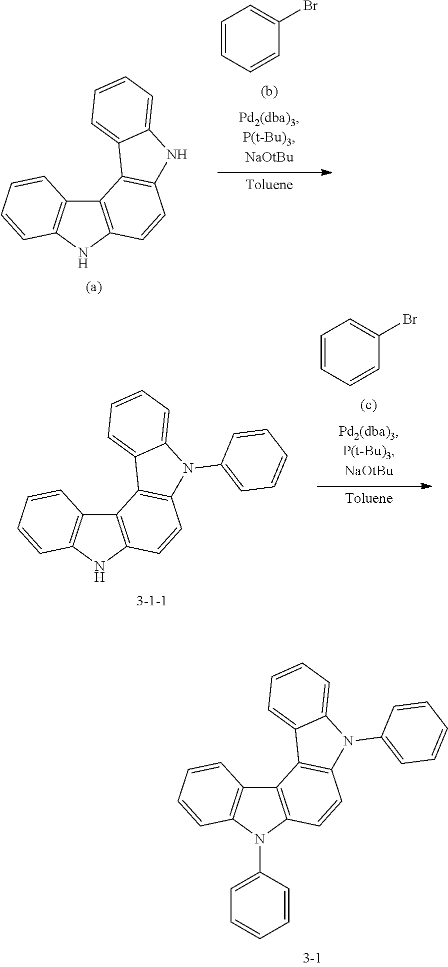 [SMILES: Brc1ccccc1 ; c1ccc2c(c1)[nH]c1ccc3[nH]c4ccccc4c3c12 ; Brc1ccccc1 ; c1ccc(-n2c3ccccc3c3c4c(ccc32)[nH]c2ccccc24)cc1 ; c1ccc(-n2c3ccccc3c3c4c5ccccc5n(-c5ccccc5)c4ccc32)cc1]

### Preparation of Compound 3-1-1

After 10 g (39.0 mmol) of 5,8-dihydroindolo[2,3-c]carbazole, 6.12 g (39.0 mmol) of 1-bromobenzene (b), 1.79 g (1.95 mmol) of tris(dibenzylideneacetone)dipalladium (Pd₂(dba)₃), 0.92 mL (3.9 mmol) of tri-tert-butylphosphine (P(t-Bu)₃), and 7.50 g (78.0 mmol) of sodium tert-butoxide (NatOBu) were put into a reaction flask, 100 mL of toluene was added thereto, and the resulting mixture was heated at 135° C. for 15 hours. When the reaction was terminated, the resulting product was extracted with methylene chloride (MC) and water, and then purified by column chromatography to obtain 7.3 g of Compound 3-1-1 (yield 56%).

### Preparation of Compound 3-1

After 7.3 g (22.0 mmol) of Compound 3-1-1, 3.8 g (24.2 mmol) of 1-bromobenzene (b), 1.01 g (1.1 mmol) of tris(dibenzylideneacetone)dipalladium (Pd₂(dba)₃), 0.52 mL (3.9 mmol) of tri-tert-butylphosphine (P(t-Bu)₃), and 4.23 g (44.0 mmol) of sodium tert-butoxide (NatOBu) were put into a reaction flask, 70 mL of toluene was added thereto, and the resulting mixture was heated at 135° C. for 15 hours. When the reaction was terminated, the resulting product was extracted with methylene chloride (MC) and water, and then purified by column chromatography to obtain 8.3 g (yield 93%) of Compound 3-1.

The target compound was synthesized in the same manner as in the preparation of Preparation Example 8, except that Intermediate A, Intermediate B, and Intermediate C in the following Table 6 were used instead of (a), (b), and (c), respectively, in Preparation Example 8.

TABLE 6

| | Intermediate A | Intermediate B | Intermediate C |
|---|---|---|---|
| 3-4 | 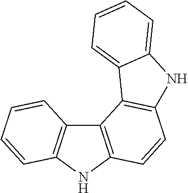 [SMILES: c1ccc2c(c1)[nH]c1ccc3[nH]c4ccccc4c3c12] | 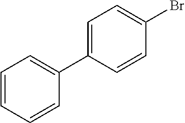 [SMILES: Brc1ccc(-c2ccccc2)cc1] | 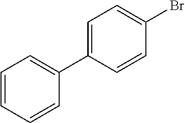 [SMILES: Brc1ccc(-c2ccccc2)cc1] |
| 3-5 | 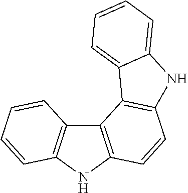 [SMILES: c1ccc2c(c1)[nH]c1ccc3[nH]c4ccccc4c3c12] | 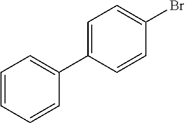 [SMILES: Brc1ccc(-c2ccccc2)cc1] | 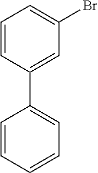 [SMILES: Brc1cccc(-c2ccccc2)c1] |
| 3-22 | 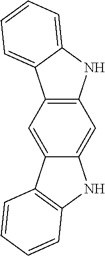 [SMILES: c1ccc2c(c1)[nH]c1cc3[nH]c4ccccc4c3cc12] | 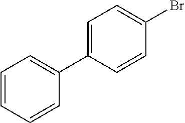 [SMILES: Brc1ccc(-c2ccccc2)cc1] | 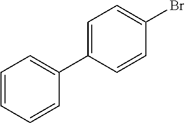 [SMILES: Brc1ccc(-c2ccccc2)cc1] |
| 3-23 | 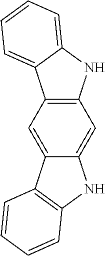 [SMILES: c1ccc2c(c1)[nH]c1cc3[nH]c4ccccc4c3cc12] | 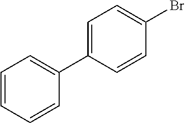 [SMILES: Brc1ccc(-c2ccccc2)cc1] | 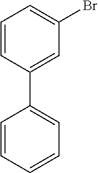 [SMILES: Brc1cccc(-c2ccccc2)c1] |
| 3-32 | 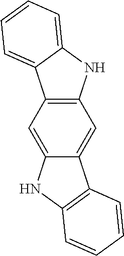 [SMILES: c1ccc2c(c1)[nH]c1cc3c(cc12)[nH]c1ccccc13] | 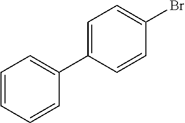 [SMILES: Brc1ccc(-c2ccccc2)cc1] | 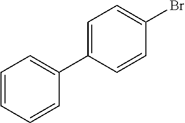 [SMILES: Brc1ccc(-c2ccccc2)cc1] |
| 3-35 | 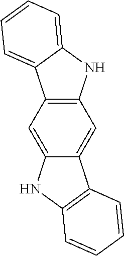 [SMILES: c1ccc2c(c1)[nH]c1cc3c(cc12)[nH]c1ccccc13] | 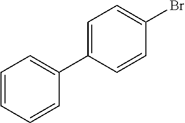 [SMILES: Brc1ccc(-c2ccccc2)cc1] | 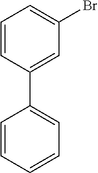 [SMILES: Brc1cccc(-c2ccccc2)c1] |
| 3-41 | 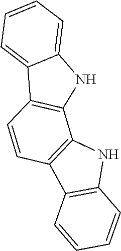 [SMILES: c1ccc2c(c1)[nH]c1c2ccc2c3ccccc3[nH]c21] | 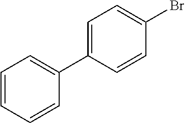 [SMILES: Brc1ccc(-c2ccccc2)cc1] | 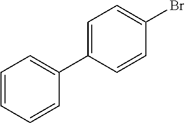 [SMILES: Brc1ccc(-c2ccccc2)cc1] |
| 3-61 | 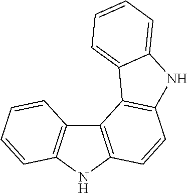 [SMILES: c1ccc2c(c1)[nH]c1ccc3[nH]c4ccccc4c3c12] | 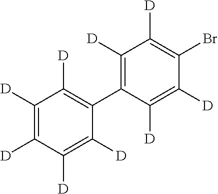 [SMILES: [2H]c1c([2H])c([2H])c(-c2c([2H])c([2H])c(Br)c([2H])c2[2H])c([2H])c1[2H]] | 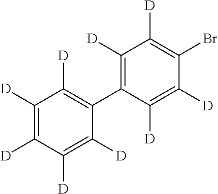 [SMILES: [2H]c1c([2H])c([2H])c(-c2c([2H])c([2H])c(Br)c([2H])c2[2H])c([2H])c1[2H]] |
| 3-69 | 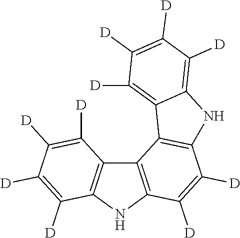 [SMILES: [2H]c1c([2H])c([2H])c2c([nH]c3c([2H])c([2H])c4[nH]c5c([2H])c([2H])c([2H])c([2H])c5c4c32)c1[2H]] | 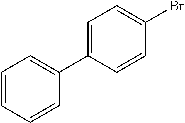 [SMILES: Brc1ccc(-c2ccccc2)cc1] | 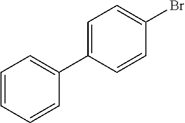 [SMILES: Brc1ccc(-c2ccccc2)cc1] |
| 3-77 | 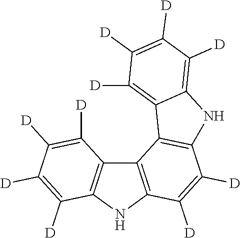 [SMILES: [2H]c1c([2H])c([2H])c2c([nH]c3c([2H])c([2H])c4[nH]c5c([2H])c([2H])c([2H])c([2H])c5c4c32)c1[2H]] | 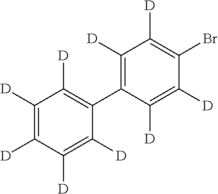 [SMILES: [2H]c1c([2H])c([2H])c(-c2c([2H])c([2H])c(Br)c([2H])c2[2H])c([2H])c1[2H]] | 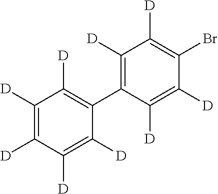 [SMILES: [2H]c1c([2H])c([2H])c(-c2c([2H])c([2H])c(Br)c([2H])c2[2H])c([2H])c1[2H]] |

| | Target |
|---|---|
| | Compound |
| 3-4 | 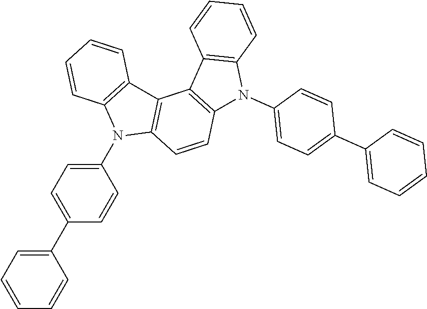 [SMILES: c1ccc(-c2ccc(-n3c4ccccc4c4c5c6ccccc6n(-c6ccc(-c7ccccc7)cc6)c5ccc43)cc2)cc1] |
| 3-5 | 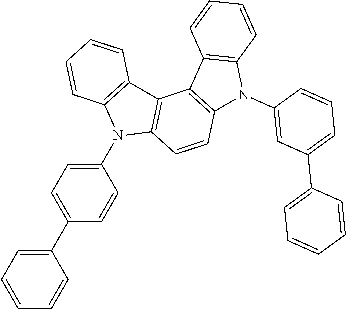 [SMILES: c1ccc(-c2ccc(-n3c4ccccc4c4c5c6ccccc6n(-c6cccc(-c7ccccc7)c6)c5ccc43)cc2)cc1] |
| 3-22 | 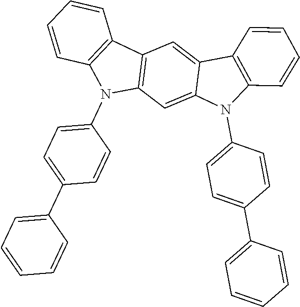 [SMILES: c1ccc(-c2ccc(-n3c4ccccc4c4cc5c6ccccc6n(-c6ccc(-c7ccccc7)cc6)c5cc43)cc2)cc1] |
| 3-23 | 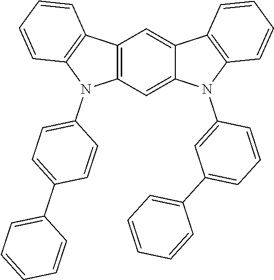 [SMILES: c1ccc(-c2ccc(-n3c4ccccc4c4cc5c6ccccc6n(-c6cccc(-c7ccccc7)c6)c5cc43)cc2)cc1] |
| 3-32 | 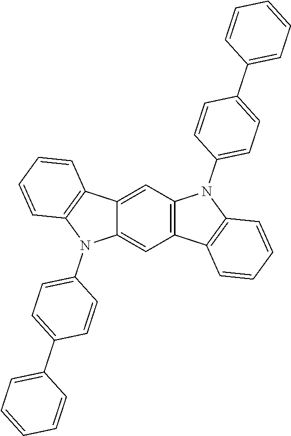 [SMILES: c1ccc(-c2ccc(-n3c4ccccc4c4cc5c(cc43)c3ccccc3n5-c3ccc(-c4ccccc4)cc3)cc2)cc1] |
| 3-35 | 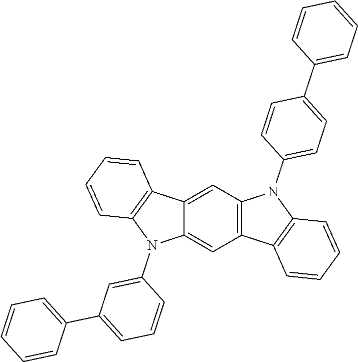 [SMILES: c1ccc(-c2ccc(-n3c4ccccc4c4cc5c(cc43)c3ccccc3n5-c3cccc(-c4ccccc4)c3)cc2)cc1] |
| 3-41 | 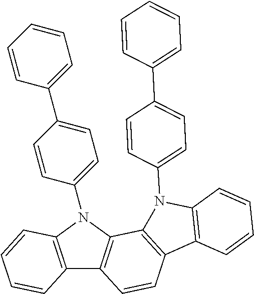 [SMILES: c1ccc(-c2ccc(-n3c4ccccc4c4ccc5c6ccccc6n(-c6ccc(-c7ccccc7)cc6)c5c43)cc2)cc1] |
| 3-61 | 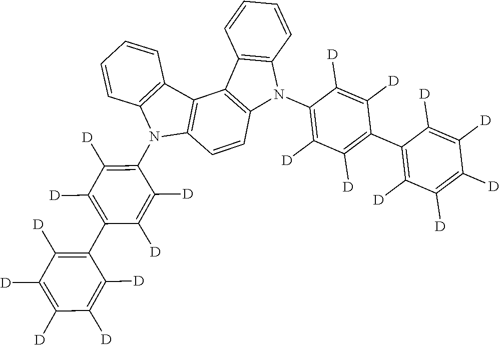 [SMILES: [2H]c1c([2H])c([2H])c(-c2c([2H])c([2H])c(-n3c4ccccc4c4c5c6ccccc6n(-c6c([2H])c([2H])c(-c7c([2H])c([2H])c([2H])c([2H])c7[2H])c([2H])c6[2H])c5ccc43)c([2H])c2[2H])c([2H])c1[2H]] |
| 3-69 | 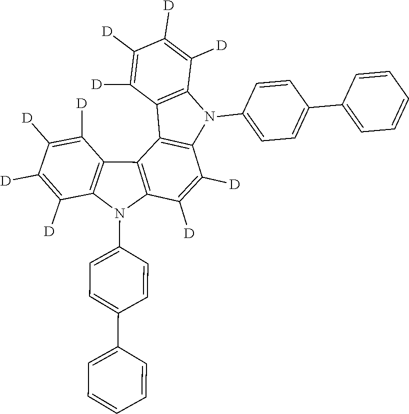 [SMILES: [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1] |
| 3-77 | 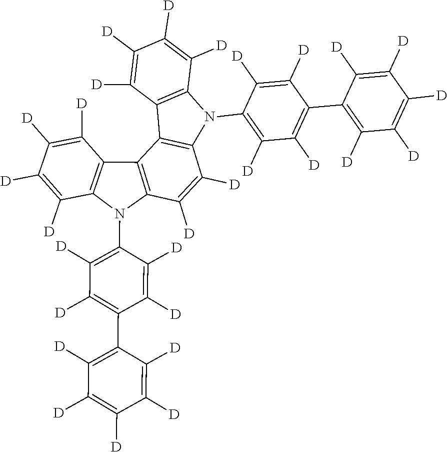 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c([2H])c43)c([2H])c2[2H])c([2H])c1[2H]] |

Compounds related to Chemical Formulae 1 to 3 were prepared in the same manner as in Preparation Examples 1 to 8 and Tables 1 to 6, and the synthesis confirmation results thereof are shown in the following Tables 7 and 8. Table 7 shows the measured values of ¹H NMR(CDCl₃, 400 Mz), and Table 8 shows the measured values of field desorption mass spectrometry (FD-MS).

TABLE 7

| Compound | |
|---|---|
| No. | ¹H NMR(CDCl₃, 400 Mz) |
| 1-1 | δ = 7.96-7.94 (4H, m), 7.79 (2H, d), 7.60 (4H, m), 7.46-7.41 (3H, t) |
| 1-5 | δ = 8.38 (1H, d), 7.94 (2H, m), 7.75-7.73 (4H, m), 7.61 (3H, m), 7.49-7.41 (3H, m) |
| 1-9 | δ = 7.96 (2H, d), 7.75 (2H, d), 7.49-7.41 (3H, m), 7.25 (6H, m) |
| 1-40 | δ = 7.96 (2H, d), 7.75 (2H, d), 7.49-7.41 (3H, m), 7.25 (10H, m) |
| 1-78 | δ = 7.96 (2H, d), 7.75 (2H, d), 7.49-7.41 (3H, m), 7.25 (14H, m) |
| 1-79 | δ = 8.08 (1H, d), 7.98 (1H, d), 7.88 (1H, d), 7.54-7.51 (2H, m), 7.39-7.31 (2H, m) |
| 1-80 | δ = 8.55 (1H, d), 8.45 (1H, d), 7.93-7.92 (2H, m), 7.70 (1H, t), 7.56-7.49 (2H, m) |
| 1-81 | δ = 8.03-7.98 (2H, m), 7.82-7.76 (2H, m), 7.54 (1H, d), 7.39-7.31 (2H, m) |
| 1-88 | δ = 7.98 (1H, d), 7.82 (1H, d), 7.69 (1H, d), 7.57-7.54 (2H, m), 7.39-7.31 (2H, m) |
| 1-93 | δ = 9.60 (1H, d), 9.27 (1H, s), 8.33-8.30 (2H, m), 8.15 (1H, d), 7.70-7.64 (4H, m), |
| | 7.52 (2H, d) |
| 1-115 | δ = 8.08-7.96 (5H, m), 7.54-7.51 (2H, m), 7.39-7.25 (4H, m) |
| 1-117 | δ = 8.03-7.96 (4H, m), 7.82-7.76 (2H, m), 7.54 (1H, d), 7.39-7.25 (4H, m) |
| 1-123 | δ = 8.09 (1H, d), 7.96-7.89 (4H, d), 7.78 (1H, d), 7.55 (1H, d), 7.38 (1H, t), 7.28-7.25 |
| | (3H, m), 1.69 (6H, s) |
| 1-177 | δ = 7.96 (2H, d), 7.75 (2H, d), 7.49-7.41 (3H, m), 7.25 (6H, m) |
| 1-208 | δ = 7.96 (2H, d), 7.75 (2H, d), 7.49-7.41 (3H, m), 7.25 (10H, m) |
| 1-211 | δ = 8.03-7.98 (2H, m), 7.82-7.76 (2H, m), 7.54 (1H, d), 7.39-7.31 (2H, m) |
| 1-298 | δ = 7.96 (2H, d), 7.75 (2H, d), 7.49-7.41 (3H, m), 7.25 (6H, m) |
| 1-301 | δ = 8.03-7.98 (2H, m), 7.82-7.76 (2H, m), 7.54 (1H, d), 7.39-7.31 (2H, m) |
| 1-398 | δ = 8.03 (2H, m), 7.82-7.75 (6H, m), 7.49-7.41 (3H, m) |
| 1-400 | δ = 8.03 (1H, d), 7.82-7.69 (6H, m), 7.57 (1H, t), 7.46-7.41 (3H, m) |
| 1-404 | δ = 7.88-7.79 (6H, m), 7.69 (1H, d), 7.57 (1H, t), 7.46-7.41 (3H, m) |
| 1-413 | δ = 8.03 (1H, d), 7.94 (1H, s), 7.82-7.41 (13H, m) |
| 1-432 | δ = 8.03-7.98 (2H, m), 7.82-7.76 (2H, m), 7.54 (1H, d), 7.39-7.31 (2H, m) |
| 1-433 | δ = 8.03-7.98 (2H, m), 7.82-7.76 (2H, m), 7.54 (1H, d), 7.39-7.31 (2H, m) |
| 1-434 | δ = 8.03-7.98 (2H, m), 7.82-7.76 (2H, m), 7.54 (1H, d), 7.39-7.31 (2H, m) |
| 1-435 | δ = 8.03 (1H, d), 7.82-7.69 (6H, m), 7.57 (1H, t), 7.46-7.41 (3H, m) |
| 1-436 | δ = 8.03 (1H, d), 7.82-7.69 (6H, m), 7.57 (1H, t), 7.46-7.41 (3H, m) |
| 1-437 | δ = 8.03 (1H, d), 7.82-7.69 (6H, m), 7.57 (1H, t), 7.46-7.41 (3H, m) |
| 2-1 | δ = 8.55(d, 1H), 8.30(d, 1H), 8.19-8.13(m, 2H), 7.99-7.89(m, 4H), 7.77(d, 1H), 7.62- |
| | 7.50(m, 12H), 7.35(t, 1H), 7.20-7.16(m, 2H) |
| 2-2 | δ = 8.55(d, 1H), 8.30(d, 1H), 8.19-8.13(m, 2H), 7.99-7.89(m, 6H), 7.80-7.77(m, 2H), |
| | 7.62-7.35(m, 10H), 7.20-7.16(m, 6H) |
| 2-3 | δ = 8.55(d, 1H), 8.18-8.09(m, 3H), 8.00-7.87(m, 3H), 7.77(s, 2H), 7.58-7.25(m, 18H) |
| 2-4 | δ = 8.55(d, 1H), 8.18-8.12(m, 2H), 8.00-7.84(m, 3H), 7.79-7.77(m, 4H), 7.68- |
| | 7.25(m, 22H) |
| 2-5 | δ = 8.55(d, 1H), 8.30(d, 1H), 8.21-8.13(m, 3H), 7.99-7.89(m, 4H), 7.77-7.35(m, |
| | 17H), 7.25-7.16(m, 6H) |
| 2-6 | δ = 8.55(d, 1H), 8.30(d, 1H), 8.19-8.13(m, 2H), 7.94-7.89(m, 8H), 7.77-7.75(m, 3H), |
| | 7.62-7.35(m, 11H), 7.25-7.16(m, 6H) |
| 2-7 | δ = 8.55(d, 1H), 8.18-8.09(m, 4H), 8.00-7.94(m, 2H), 7.87(m, 1H), 7.77(m, 2H), |
| | 7.69-7.63(m, 2H), 7.52-7.25(m, 20H) |
| 2-11 | δ = 8.55(d, 1H), 8.30(d, 1H), 8.19-8.13(m, 2H), 7.99-7.89(m, 5H), 7.77(d, 1H), 7.58- |
| | 7.28(m, 16H), 1.69(s, 6H) |
| 2-16 | δ = 9.05(s, 1H), 8.55(d, 1H), 8.33-8.13(m, 7H), 7.99-7.89(m, 5H), 7.77-7.50(m, |
| | 13H), 7.35(t, 1H), 7.20-7.16(m, 2H) |
| 2-19 | δ = 8.55(d, 1H), 8.30(d, 1H), 8.19-8.13(m, 2H), 7.99-7.89(m, 8H), 7.80-7.77(m, 3H), |
| | 7.58(d, 1H), 7.50-7.35(m, 6H), 7.20-7.16(m, 10H) |
| 2-20 | δ = 8.55(d, 1H), 8.30(d, 1H), 8.21-8.13(m, 3H), 7.99-7.89(m, 6H), 7.80-7.35(m, |
| | 15H), 7.20-7.16(6H) |
| 2-21 | 8 -8.55(d, 1H), 8.30(d, 1H), 8.19-8.13(m, 2h), 7.99-7.89(m, 10H), 7.80-7.75(m, |
| | 4H), 7.50-7.35(m, 8H), 7.20-7.16(m, 6H) |
| 2-22 | δ = 8.55(d, 1H), 8.30(d, 1H), 8.21-8.13(m, 3H), 7.99-7.89(m, 6H), 7.80-7.35(m, |
| | 15H), 7.25-7.16(10H) |
| 2-23 | δ = 8.55(d, 1H), 8.30(d, 1H), 8.19-8.13(m, 2h), 7.99-7.89(m, 10H), 7.80-7.75(m, |
| | 4H), 7.50-7.35(m, 8H), 7.25-7.16(m, 10H) |
| 2-26 | δ = 8.55(m, 1H), 8.30(d, 1H), 8.21-8.13(m, 4h), 7.99-7.89(m, 4H), 7.77-7.35(m, |
| | 20H), 7.25-7.16(6H) |
| 2-27 | δ = 8.55(m, 1H), 8.30(d, 1H), 8.21-8.13(m, 4h), 7.99-7.89(m, 4H), 7.77-7.35(m, |
| | 20H), 7.20-7.16(2H) |
| 2-28 | δ = 8.55(m, 1H), 8.18-8.09(m, 3H), 8.00-8.79(m, 2H), 7.87(m, 1H), 7.79-7.77(m, |
| | 4H), 7.69-7.63(m, 4H), 7.52-7.25(m, 12H) |
| 2-29 | δ = 8.55(m, 1H), 8.18-8.09(m, 3H), 8.00-7.94(m, 2H0, 7.87(m, 1H), 7.87(m, 1H), |
| | 7.79-7.77(m, 4H), 7.69-7.63(m, 4H), 7.52-7.25(m, 21H) |
| 2-30 | δ = 8.55(m, 1H), 8.31-8.30(m, 3H), 8.21-8.13(m, 3h), 7.99-7.89(m, 3H), 7.75- |
| | 7.35(m, 22H), 7.20-7.16(m, 2H) |
| 2-32 | δ = 8.55(m, 1H), 8.18-8.12(m, 2H), 8.00-7.87(m, 3H), 7.79-7.77(m, 6H), 7.69- |
| | 7.63(m, 6H), 7.52-7.25(m, 14H) |
| 2-33 | δ = 8.55(m, 1H), 8.30(d, 1H), 8.21-8.13(m, 3H), 7.99-7.89(m, 8H), 7.77-7.35(m, |
| | 17H), 7.25-7.16(6H) |
| 2-34 | δ = 8.55(m, 1H), 8.18-8.12(m, 2H), 8.00-7.87(m, 3H), 7.79-7.77(m, 6H), 7.67- |
| | 7.63(m, 6H), 7.52-7.25(m, 18H) |
| 2-38 | δ = 8.55(m, 1H), 8.18-8.12(m, 2H), 8.05-7.87(m, 6H), 7.79-7.77(m, 4H), 7.69- |
| | 7.63(m, 4H), 7.52-7.25(m, 23H) |
| 2-40 | δ = 8.55(m, 1H), 8.30(d, 1H), 8.19-8.13(m, 2H), 8.03-7.75(m, 15H), 7.58-7.35(m, |
| | 9H), 7.25-7.16(m, 6H) |
| 2-41 | δ = 8.55(m, 1H), 8.30(d, 1H), 8.19-8.13(m, 2H), 7.99-7.89(m, 4H), 7.77(d, 1H), |
| | 7.58-7.50(m, 2H), 7.35(t, 1H), 7.20-7.16(m, 2H) |
| 2-42 | δ = 8.55(m, 1H), 8.30(d, 1H), 8.19-8.13(m, 2H), 7.99-7.89(m, 4H), 7.77(d, 1H), |
| | 7.58-7.50(m, 2H), 7.35(t, 1H), 7.20-7.16(m, 2H) |
| 2-43 | δ = 8.55(m, 1H), 8.30(d, 1H), 8.19-8.13(m, 2H), 7.99-7.89(m, 4H), 7.77(d, 1H), |
| | 7.58-7.50(m, 2H), 7.35(t, 1H), 7.20-7.16(m, 2H) |
| 2-45 | δ = 8.55(m, 1H), 8.30(d, 1H), 8.19-8.13(m, 2H), 7.99-7.89(m, 4H), 7.77(d, 1H), |
| | 7.58-7.50(m, 2H), 7.35(t, 1H), 7.20-7.16(m, 2H) |
| 2-46 | δ = 8.55(m, 1H), 8.30(d, 1H), 8.19-8.13(m, 2H), 7.99-7.89(m, 4H), 7.77(d, 1H), |
| | 7.58-7.50(m, 2H), 7.35(t, 1H), 7.20-7.16(m, 2H) |
| 2-48 | δ = 8.55(m, 1H), 8.30(d, 1H), 8.19-8.13(m, 2H), 7.99-7.89(m, 4H), 7.77(d, 1H), |
| | 7.58-7.50(m, 2H), 7.35(t, 1H), 7.20-7.16(m, 2H) |
| 2-49 | δ = 8.55(m, 1H), 8.30(d, 1H), 8.19-8.13(m, 2H), 7.99-7.89(m, 4H), 7.77(d, 1H), |
| | 7.58-7.50(m, 2H), 7.35(t, 1H), 7.20-7.16(m, 2H) |
| 2-50 | δ = 8.55(m, 1H), 8.30(d, 1H), 8.19-8.13(m, 2H), 7.99-7.89(m, 4H), 7.77(d, 1H), |
| | 7.58-7.50(m, 2H), 7.35(t, 1H), 7.20-7.16(m, 2H) |
| 2-51 | δ = 8.55(m, 1H), 8.30(d, 1H), 8.19-8.13(m, 2H), 7.99-7.89(m, 4H), 7.77(d, 1H), |
| | 7.58-7.50(m, 2H), 7.35(t, 1H), 7.20-7.16(m, 2H) |
| 2-52 | δ = 8.55(m, 1H), 8.30(d, 1H), 8.19-8.13(m, 2H), 7.99-7.89(m, 4H), 7.77(d, 1H), |
| | 7.58-7.50(m, 2H), 7.35(t, 1H), 7.20-7.16(m, 2H) |
| 2-55 | δ = 8.55(m, 1H), 8.30(d, 1H), 8.19-8.13(m, 2H), 7.99-7.89(m, 4H), 7.77(d, 1H), |
| | 7.58-7.50(m, 2H), 7.35(t, 1H), 7.20-7.16(m, 2H) |
| 2-57 | δ = 8.55(m, 1H), 8.30(d, 1H), 8.19-8.13(m, 2H), 7.99-7.89(m, 4H), 7.77(d, 1H), |
| | 7.58-7.50(m, 2H), 7.35(t, 1H), 7.20-7.16(m, 2H) |
| 2-60 | δ = 8.55(m, 1H), 8.30(d, 1H), 8.19-8.13(m, 2H), 7.99-7.89(m, 4H), 7.77(d, 1H), |
| | 7.58-7.50(m, 2H), 7.35(t, 1H), 7.20-7.16(m, 2H) |
| 2-61 | δ = 7.62-7.50(m, 10H) |
| 2-62 | δ = 7.79(m, 4H), 7.68(m. 4H), 7.52-7.41(m, 10H) |
| 2-63 | δ = 8.21(s, 1H) 7.75-7.41(m, 13H) |
| 2-64 | δ = 9.05(s, 1H), 8.33-8.25(m, 4H), 7.94(d, 1H), 7.70-7.50(m, 10H) |
| 2-65 | δ = 7.79(m, 2H), 7.70-7.68(m, 3H), 7.58-7.41(m, 13H) |
| 2-66 | δ = 7.92-7.91(m, 4H), 7.75(d, 2H), 7.62-7.41(m, 8H), 7.25(s, 4H) |
| 2-68 | δ = 8.21(s, 2H), 7.75-7.60(m, 8H), 7.49-7.41(8H) |
| 2-69 | δ = 8.21(s, 1H), 7.92-7.91(m, 4H), 7.75-7.60(m, 6H), 7.49-7.41(m, 7H) |
| 2-70 | δ = 8.21(s, 1H), 7.94-7.91(m, 5H), 7.75-7.61(m, 9H), 7.49-7.41(m, 7H) |
| 2-74 | δ = 7.94-7.91(m, 9H), 7.75-7.73(m, 5H), 7.61(d, 2H), 7.49-7.41(m, 6H) |
| 2-75 | δ = 7.92-7.91(m, 8H), 7.75(d, 4H), 7.49-7.41(m, 6H), 7.25(s, 4H) |
| 3-1 | δ = 8.55(2H, d), 7.94(2H, d), 7.62-7.35(14H, m), 7.16(2H, d) |
| 3-4 | δ = 8.55(2H, d), 7.94-7.91(10H, m), 7.75(4H, d), 7.49-7.35(10H, m), 7.16(2H, t) |
| 3-5 | δ = 8.55(2H, d), 8.21(1H, s), 7.94-7.91(6H, m), 7.75-7.35(16H, m), 7.26(1H, d), |
| | 7.16(2H, t) |
| 3-22 | δ = 8.55(1H, d), 8.19(1H, d), 7.94-7.91(9H, m), 7.75(4H, d), 7.58-7.35(11H, m), |
| | 7.20-7.16(2H, m) |
| 3-23 | δ = 8.55(1H, d), 8.21-8.19(2H, m), 7.94-7.91(5H, m), 7.75-7.35(18H, m), 7.20- |
| | 7.16(2H, m) |
| 3-32 | δ = 8.55(1H, d), 8.19(1H, d), 7.94-7.91(9H, m), 7.75(4H, d), 7.58-7.35(11H, m), |
| | 7.20-7.16(2H, m) |
| 3-35 | δ = 8.55(1H, d), 8.21-8.19(2H, m), 7.94-7.91(5H, m), 7.68-7.35(18H, m), 7.20- |
| | 7.16(2H, m) |
| 3-41 | δ = 8.55(2H, d), 7.94-7.91(10H, m), 7.84(2H, d), 7.75(4H, d), 7.49-7.35(8H, m), |
| | 7.16(2H, t) |
| 3-61 | δ = 8.55(2H, d), 7.94(2H, d), 7.42-7.35(4H, m), 7.16(2H, t) |
| 3-69 | δ = 7.92-7.91(8H, m), 7.75(4H, d), 7.49-7.41(6H, m) |

TABLE 8

| Compound | FD-MS | Compound | FD-MS |
|---|---|---|---|
| 1-1 | m/z = 655.34 | 1-5 | m/z = 655.34 |
| | (C₄₅H₁₃D₁₆N₅ = 655.86) | | (C₄₅H₁₃D₁₆N₅ = 655.86) |
| 1-9 | m/z = 655.34 | 1-40 | m/z = 731.37 |
| | (C₄₅H₁₃D₁₆N₅ = 655.86) | | (C₅₁H₁₇D₁₆N₅ = 731.96) |
| 1-78 | m/z = 807.41 | 1-79 | m/z = 593.29 |
| | (C₅₇H₂₁D₁₆N₅ = 808.06) | | (C₃₉H₇D₁₆N₅O = 593.74) |
| 1-80 | m/z = 609.27 | 1-81 | m/z = 593.29 |
| | (C₃₉H₇D₁₆N₅S = 609.81) | | (C₃₉H₇D₁₆N₅O = 593.74) |
| 1-88 | m/z = 593.29 | 1-115 | m/z = 669.32 |
| | (C₃₉H₇D₁₆N₅O = 593.74) | | (C₄₅H₁₁D₁₆N₅O = 669.84) |
| 1-117 | m/z = 669.32 | 1-123 | m/z = 695.37 |
| | (C₄₅H₁₁D₁₆N₅O = 669.84) | | (C₄₈H₁₇D₁₆N₅ = 695.92) |
| 1-177 | m/z = 735.40 | 1-208 | m/z = 811.43 |
| | (C₅₁H₁₃D₂₀N₅ = 735.98) | | (C₅₇H₁₇D₂₀N₅ = 812.08) |
| 1-211 | m/z = 673.35 | 1-298 | m/z = 815.46 |
| | (C₄₅H₇D₂₀N₅O = 673.87) | | (C₅₇H₁₃D₂₄N₅ = 816.10) |
| 1-301 | m/z = 753.40 | 1-398 | m/z = 669.32 |
| | (C₅₁H₇D₂₄N₅O = 753.99) | | (C₄₅H₁₁D₁₆N₅O = 669.84) |
| 1-400 | m/z = 669.32 | 1-404 | m/z = 669.32 |
| | (C₄₅H₁₁D₁₆N₅O = 669.84) | | (C₄₅H₁₁D₁₆N₅O = 669.84) |
| 1-413 | m/z = 745.35 | 1-432 | m/z = 583.23 |
| | (C₅₁H₁₅D₁₆N₅O = 745.94) | | (C₃₉H₁₇D₆N₅O = 583.68) |
| 1-433 | m/z = 585.24 | 1-434 | m/z = 591.28 |
| | (C₃₉H₁₅D₈N₅O = 585.70) | | (C₃₉H₉D₁₄N₅O = 591.73) |
| 1-435 | m/z = 659.26 | 1-436 | m/z = 661.27 |
| | (C₄₅H₂₁D₆N₅O = 659.78) | | (C₄₅H₁₉D₈N₅O = 661.79) |
| 1-437 | m/z = 665.30 | | |
| | (C₄₅H₁₅D₁₂N₅O = 665.82) | | |
| 2-1 | m/z = 484.59(C₃₆H₂₄N₂ = 484.19) | 2-2 | m/z = |
| | | | 560.69(C₄₂H₂₈N₂ = 560.23) |
| 2-3 | m/z = 560.69(C₄₂H₂₈N₂ = 560.23) | 2-4 | m/z = |
| | | | 560.69(C₄₂H₂₈N₂ = 560.23) |
| 2-5 | m/z = 636.78(C₄₈H₃₂N₂ = 636.26) | 2-6 | m/z = |
| | | | 636.78(C₄₈H₃₂N₂ = 636.26) |
| 2-7 | m/z = 636.78(C₄₈H₃₂N₂ = 636.26) | 2-8 | m/z = |
| | | | 543.65(C₄₀H₂₆N₂ = 543.21) |
| 2-9 | m/z = 543.65(C₄₀H₂₆N₂ = 543.21) | 2-10 | m/z = |
| | | | 600.75(C₄₅H₃₅N₂ = 600.26) |
| 2-11 | m/z = 600.75(C₄₅H₃₅N₂ = 600.26) | 2-12 | m/z = |
| | | | 724.89(C₅₅H₃₆N₂ = 724.29) |
| 2-13 | m/z = 724.89(C₅₅H₃₆N₂ = 724.29) | 2-14 | m/z = |
| | | | 724.89(C₅₅H₃₆N₂ = 724.29) |
| 2-15 | m/z = 724.89(C₅₅H₃₆N₂ = 724.29) | 2-16 | m/z = |
| | | | 634.77(C48H30N2 = 634.24) |
| 2-17 | m/z = 509.60(C₃₇H₂₃N₃ = 509.19) | 2-18 | m/z = |
| | | | 742.98(C₅₄H₃₈N₂Si = 742.28) |
| 2-19 | m/z = 636.78(C₄₈H₃₂N₂ = 636.26) | 2-20 | m/z = |
| | | | 636.78(C₄₈H₃₂N₂ = 636.26) |
| 2-21 | m/z = 636.78(C₄₈H₃₂N₂ = 636.26) | 2-22 | m/z = |
| | | | 712.88(C₅₄H₃₆N₂ = 712.29) |
| 2-23 | m/z = 712.88(C₅₄H₃₆N₂ = 712.29) | 2-24 | m/z = |
| | | | 712.88(C₅₄H₃₆N₂ = 712.29) |
| 2-25 | m/z = 710.86(C₅₄H₃₄N₂ = 710.27) | 2-26 | m/z = |
| | | | 712.88(C₅₄H₃₆N₂ = 712.29) |
| 2-27 | m/z = 712.88(C₅₄H₃₆N₂ = 712.29) | 2-28 | m/z = |
| | | | 712.88(C₅₄H₃₆N₂ = 712.29) |
| 2-29 | m/z = 712.88(C₅₄H₃₆N₂ = 712.29) | 2-30 | m/z = |
| | | | 712.88(C₅₄H₃₆N₂ = 712.29) |
| 2-31 | m/z = 710.86(C₅₄H₃₄N₂ = 710.27) | 2-32 | m/z = |
| | | | 636.78(C₄₈H₃₂N₂ = 636.26) |
| 2-33 | m/z = 712.88(C₅₄H₃₆N₂ = 712.29) | 2-34 | m/z = |
| | | | 712.88(C₅₄H₃₆N₂ = 712.29) |
| 2-35 | m/z = 788.97(C₆₀H₄₀N₂ = 788.32) | 2-36 | m/z = |
| | | | 686.84(C₅₂H₃₄N₂ = 686.27) |
| 2-37 | m/z = 788.97(C₆₀H₄₀N₂ = 788.32) | 2-38 | m/z = |
| | | | 788.97(C₆₀H₄₀N₂ = 788.32) |
| 2-39 | m/z = 686.84(C₅₂H₃₄N₂ = 686.27) | 2-40 | m/z = |
| | | | 686.84(C₅₂H₃₄N₂ = 686.27) |
| 2-41 | m/z = 494.65(C₃₆H₁₄D₁₀N₂ = 494.26) | 2-42 | m/z = |
| | | | 654.89(C₄₈H₁₄D₁₈N₂ = 654.37) |
| 2-43 | m/z = 574.77(C₄₁H₁₄D₁₄N₂ = 574.31) | 2-44 | m/z = |
| | | | 650.86(C₄₈H₁₄D₁₆N₂ = 650.34) |
| 2-45 | m/z = 654.89(C₄₈H₁₄D₁₈N₂ = 654.37) | 2-46 | m/z = |
| | | | 654.89(C₄₈H₁₄D₁₈N₂ = 654.37) |
| 2-47 | m/z = 654.89(C₄₈H₁₄D₁₈N₂ = 654.37) | 2-48 | m/z = |
| | | | 654.89(C₄₈H₁₄D₁₈N₂ = 654.37) |
| 2-49 | m/z = 654.89(C₄₈H₁₄D₁₈N₂ = 654.37) | 2-50 | m/z: |
| | | | 734.43(C₅₄H₁₄D₂₂N₂ = 735.03) |
| 2-51 | m/z = 735.01(C₅₄H₁₄D₂₂N₂ = 734.43) | 2-52 | m/z = |
| | | | 735.01(C₅₄H₁₄D₂₂N₂ = 734.43) |
| 2-53 | m/z = 730.98(C₅₄H₁₄D₂₀N₂ = 730.40) | 2-54 | m/z = |
| | | | 735.01(C₅₄H₁₄D₂₂N₂ = 734.43) |
| 2-55 | m/z = 735.01(C₅₄H₁₄D₂₂N₂ = 734.43) | 2-56 | m/z = |
| | | | 815.13(C₆₀H₁₄D₂₆N₂ = 814.48) |
| 2-57 | m/z = 815.13(C₆₀H₁₄D₂₆N₂ = 814.48) | 2-58 | m/z = |
| | | | 815.13(C₆₀H₁₄D₂₆N₂ = 814.48) |
| 2-59 | m/z = 815.13(C₆₀H₁₄D₂₆N₂ = 814.48) | 2-60 | m/z = |
| | | | 666.86(C₄₈H₁₄D₁₆N₂O = 666.34) |
| 2-61 | m/z = 498.68(C₃₆H₁₀D₁₄N₂ = 498.28) | 2-62 | m/z = |
| | | | 650.87(C₄₈H₁₈D₁₄N₂ = 650.34) |
| 2-63 | m/z = 574.77(C₄₁H₁₄D₁₄N₂ = 574.31) | 2-64 | m/z = |
| | | | 648.85(C₄₈H₁₆D₁₄N₂ = 648.33) |
| 2-65 | m/z = 650.87(C₄₈H₁₈D₁₄N₂ = 650.34) | 2-66 | m/z = |
| | | | 650.87(C₄₈H₁₈D₁₄N₂ = 650.34) |
| 2-67 | m/z = 650.87(C₄₈H₁₈D₁₄N₂ = 650.34) | 2-68 | m/z = |
| | | | 650.87(C₄₈H₁₈D₁₄N₂ = 650.34) |
| 2-69 | m/z = 650.87(C₄₈H₁₈D₁₄N₂ = 650.34) | 2-70 | m/z = |
| | | | 726.38(C₅₄H₂₂D₁₄N₂ = 726.98) |
| 2-71 | m/z = 726.96(C₅₄H₂₂D₁₄N₂ = 726.38) | 2-72 | m/z = |
| | | | 726.96(C₅₄H₂₂D₁₄N₂ = 726.38) |
| 2-73 | m/z = 724.95(C₅₄H₂₀D₁₄N₂ = 724.36) | 2-74 | m/z = |
| | | | 726.96(C₅₄H₂₂D₁₄N₂ = 726.38) |
| 2-75 | m/z = 726.96(C₅₄H₂₂D₁₄N₂ = 726.38) | 2-76 | m/z = |
| | | | 803.06(C₆₀H₂₆D₁₄N₂ = 802.41) |
| 2-77 | m/z = 803.06(C₆₀H₂₆D₁₄N₂ = 802.41) | 2-78 | m/z = |
| | | | 803.06(C₆₀H₂₆D₁₄N₂ = 802.41) |
| 2-79 | m/z = 803.06(C₆₀H₂₆D₁₄N₂ = 802.41) | 2-80 | m/z = |
| | | | 803.06(C₆₀H₂₆D₁₄N₂ = 802.41) |
| 2-81 | m/z = 508.74(C₃₆D₂₄N₂ = 508.34) | 2-82 | m/z = |
| | | | 668.98(C₄₈D₃₂N₂ = 668.46) |
| 2-83 | m/z = 588.86(C₄₂D₂₈N₂ = 588.40) | 2-84 | m/z = |
| | | | 664.95(C₄₈D₃₀N₂ = 664.43) |
| 2-85 | m/z = 668.98(C₄₈D₃₂N₂ = 668.46) | 2-86 | m/z = |
| | | | 668.98(C₄₈D₃₂N₂ = 668.46) |
| 2-87 | m/z = 668.98(C₄₈D₃₂N₂ = 668.46) | 2-88 | m/z = |
| | | | 668.98(C₄₈D₃₂N₂ = 668.46) |
| 2-89 | m/z = 668.98(C₄₈D₃₂N₂ = 668.46) | 2-90 | m/z = |
| | | | 748.518(C₅₄D₃₆N₂ = 749.12) |
| 2-91 | m/z = 749.10(C₅₄D₃₆N₂ = 748.51) | 2-92 | m/z = |
| | | | 749.10(C₅₄D₃₆N₂ = 748.51) |
| 2-93 | m/z = 745.07(C₅₄D₃₄N₂ = 744.49) | 2-94 | m/z = |
| | | | 749.10(C₅₄D₃₆N₂ = 748.51) |
| 2-95 | m/z = 749.10(C₅₄D₃₆N₂ = 748.51) | 2-96 | m/z = |
| | | | 829.22(C₆₀D₄₀N₂ = 828.57) |
| 2-97 | m/z = 829.22(C₆₀D₄₀N₂ = 828.57) | 2-98 | m/z = |
| | | | 829.22(C₆₀D₄₀N₂ = 828.57) |
| 2-99 | m/z = 829.22(C₆₀D₄₀N₂ = 828.57) | 2-100 | m/z = |
| | | | 680.95(C₄₈D₃₀N₂O = 680.42) |
| 2-101 | m/z = 697.02(C₄₈D₃₀N₂S = 696.40) | 2-102 | m/z = |
| | | | 829.22(C₆₀D₄₀N₂ = 828.57) |
| 2-103 | m/z = 632.95(C₄₅D₃₂N₂ = 632.46) | 2-104 | m/z = |
| | | | 713.07(C₅₁D₃₆N₂ = 712.51) |
| 3-1 | m/z = 408.16(C₃₀H₂₀N₂ = 408.50) | 3-4 | m/z = |
| | | | 560.23(C₄₂H₂₈N₂ = 560.70) |
| 3-5 | m/z = m/z = | 3-22 | m/z = m/z = |
| | 560.23(C₄₂H₂₈N₂ = 560.70) | | 560.23(C₄₂H₂₈N₂ = 560.70) |
| 3-23 | m/z = m/z = | 3-32 | m/z = m/z = |
| | 560.23(C₄₂H₂₈N₂ = 560.70) | | 560.23(C₄₂H₂₈N₂ = 560.70) |
| 3-35 | m/z = m/z = | 3-41 | m/z = m/z = |
| | 560.23(C₄₂H₂₈N₂ = 560.70) | | 560.23(C₄₂H₂₈N₂ = 560.70) |
| 3-61 | m/z = 578.34(C₄₂H₁₀D₁₈N₂ = 578.81) | 3-69 | m/z = |
| | | | 570.29(C₄₂H₁₈D₁₀N₂ = 570.76) |
| 3-77 | m/z = 588.40(C₄₂D₂₈N₂ = 588.87) | | |

### <Experimental Example 1>—Manufacture of Organic Light Emitting Device

A glass substrate thinly coated with indium tin oxide (ITO) having a thickness of 1,500 Å was ultrasonically washed with distilled water. When the washing with distilled water was finished, the glass substrate was ultrasonically washed with a solvent such as acetone, methanol, and isopropyl alcohol, dried and then subjected to ultraviolet ozone (UVO) treatment for 5 minutes using UV in an ultraviolet (UV) washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4′,4″-tris[2-naphthyl(phenyl)amino]triphenylamine (2-TNATA) and the hole transport layer N,N′-di(1-naphthyl)-N,N′-diphenyl-(1,1′-biphenyl)-4,4′-diamine (NPB) were formed on the ITO transparent electrode (positive electrode).

A light emitting layer was thermally vacuum deposited thereon as follows. The light emitting layer was deposited to have a thickness of 400 Å by using the compound of Chemical Formula 1 shown in the following Table 9 as a host, and was deposited by doping the host with Ir(ppy)3 as a green phosphorescent dopant to 7% of the deposition thickness of the light emitting layer. Thereafter, bathocuproine (BCP) as a hole blocking layer was deposited to have a thickness of 60 Å, and Alq3 as an electron transport layer was deposited to have a thickness of 200 Å thereon. Finally, lithium fluoride (LiF) was deposited to have a thickness of 10 Å on the electron transport layer to form an electron injection layer, and then an aluminum (Al) negative electrode was deposited to have a thickness of 1,200 Å on the electron injection layer to form a negative electrode, thereby manufacturing an organic electroluminescence device.

Meanwhile, all the organic compounds required for manufacturing an OLED device were subjected to vacuum sublimed purification under 10⁻⁶ to 10⁻⁸ torr for each material, and used for the manufacture of OLED.

For the organic electroluminescence device manufactured as described above, electroluminescence (EL) characteristics were measured by M7000 manufactured by McScience Inc., and based on the measurement result thereof, T₉₀ was measured by a service life measurement device (M6000) manufactured by McScience Inc., when the reference luminance was 6,000 cd/m².

The results of measuring the driving voltage, light emitting efficiency, color (EL color), and service life of the organic light emitting device manufactured according to the present invention are shown in the following Table 9.

TABLE 9

| | Light | Driving | | | Service |
|---|---|---|---|---|---|
| | emitting | voltage | Efficiency | | life |
| | compound | (V) | (cd/A) | EL color | (T₉₀) |
| Example 1 | 1-1 | 4.12 | 80.2 | Green | 130 |
| Example 2 | 1-5 | 4.38 | 74.8 | | 142 |
| Example 3 | 1-9 | 4.38 | 74.7 | | 199 |
| Example 4 | 1-40 | 4.38 | 74.8 | | 203 |
| Example 5 | 1-78 | 4.39 | 74.8 | | 201 |
| Example 6 | 1-79 | 4.39 | 74.8 | | 156 |
| Example 7 | 1-80 | 4.38 | 74.7 | | 153 |
| Example 8 | 1-81 | 4.38 | 74.7 | | 196 |
| Example 9 | 1-88 | 4.06 | 75.1 | | 163 |
| Example 10 | 1-93 | 4.15 | 76.9 | | 156 |
| Example 11 | 1-115 | 4.05 | 83.2 | | 147 |
| Example 12 | 1-117 | 4.41 | 69.5 | | 153 |
| Example 13 | 1-123 | 4.10 | 82.8 | | 145 |
| Example 14 | 1-177 | 4.18 | 76.4 | | 209 |
| Example 15 | 1-208 | 4.09 | 83.2 | | 215 |
| Example 16 | 1-211 | 4.05 | 83.2 | | 206 |
| Example 17 | 1-298 | 4.07 | 77.9 | | 229 |
| Example 18 | 1-301 | 4.39 | 74.8 | | 220 |
| Example 19 | 1-398 | 4.01 | 71.1 | | 218 |
| Example 20 | 1-400 | 3.98 | 69.2 | | 229 |
| Example 21 | 1-404 | 3.72 | 67.8 | | 207 |
| Example 22 | 1-413 | 4.08 | 73.3 | | 219 |
| Example 23 | 1-432 | 4.53 | 64.3 | | 147 |
| Example 24 | 1-433 | 4.46 | 68.6 | | 163 |
| Example 25 | 1-434 | 4.42 | 71.7 | | 185 |
| Example 26 | 1-435 | 4.18 | 63.8 | | 173 |
| Example 27 | 1-436 | 4.02 | 65.3 | | 202 |
| Example 28 | 1-437 | 3.98 | 67.2 | | 217 |
| Example 29 | 1-126 | 4.18 | 70.4 | | 184 |
| Example 30 | 1-354 | 4.35 | 73.7 | | 163 |
| Comparative | Ref. 1 | 4.78 | 55.1 | | 101 |
| Example 1 | | | | | |
| Comparative | Ref. 2 | 4.77 | 56.2 | | 130 |
| Example 2 | | | | | |
| Comparative | Ref. 3 | 4.34 | 51.2 | | 146 |
| Example 3 | | | | | |
| Comparative | Ref. 4 | 4.68 | 57.7 | | 81 |
| Example 4 | | | | | |
| Comparative | Ref. 5 | 5.52 | 49.2 | | 59 |
| Example 5 | | | | | |
| Comparative | Ref. 6 | 5.49 | 43.1 | | 53 |
| Example 6 | | | | | |
| Comparative | Ref. 7 | 5.69 | 46.6 | | 54 |
| Example 7 | | | | | |
| Comparative | Ref. 8 | 4.72 | 47.4 | | 68 |
| Example 8 | | | | | |
| Comparative | Ref. 9 | 4.99 | 49.3 | | 34 |
| Example 9 | | | | | |
| Comparative | Ref. 10 | 4.38 | 50.6 | | 134 |
| Example 10 | | | | | |
| Comparative | Ref. 11 | 4.51 | 50.2 | | 129 |
| Example 11 | | | | | |
| Comparative | Ref. 12 | 4.78 | 45.4 | | 64 |
| Example 12 | | | | | |
| Comparative | Ref. 13 | 4.85 | 52.2 | | 101 |
| Example 13 | | | | | |
| Comparative | Ref. 14 | 4.56 | 50.2 | | 113 |
| Example 14 | | | | | |

Ref. 1 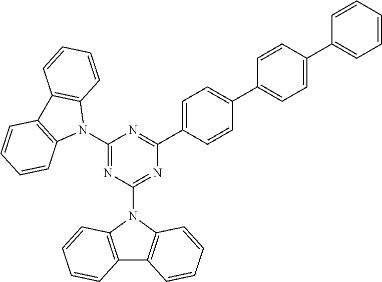 [SMILES: c1ccc(-c2ccc(-c3ccc(-c4nc(-n5c6ccccc6c6ccccc65)nc(-n5c6ccccc6c6ccccc65)n4)cc3)cc2)cc1]
Ref. 2 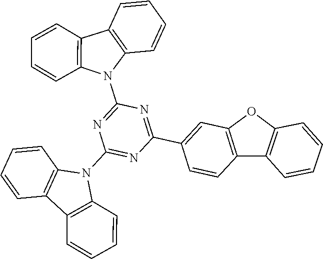 [SMILES: c1ccc2c(c1)oc1cc(-c3nc(-n4c5ccccc5c5ccccc54)nc(-n4c5ccccc5c5ccccc54)n3)ccc12]
Ref. 3 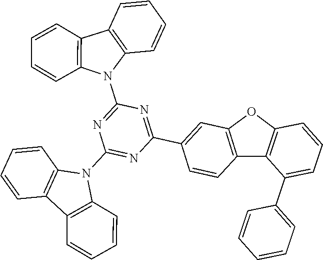 [SMILES: c1ccc(-c2cccc3oc4cc(-c5nc(-n6c7ccccc7c7ccccc76)nc(-n6c7ccccc7c7ccccc76)n5)ccc4c23)cc1]
Ref. 4 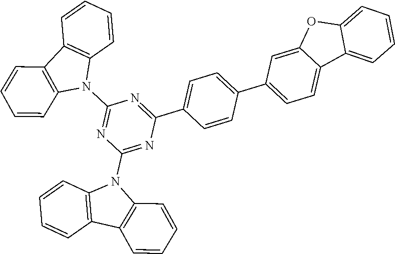 [SMILES: c1ccc2c(c1)oc1cc(-c3ccc(-c4nc(-n5c6ccccc6c6ccccc65)nc(-n5c6ccccc6c6ccccc65)n4)cc3)ccc12]
Ref. 5 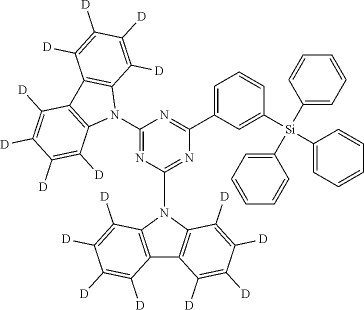 [SMILES: [2H]c1c([2H])c([2H])c2c(c1[2H])c1c([2H])c([2H])c([2H])c([2H])c1n2-c1nc(-c2cccc([Si](c3ccccc3)(c3ccccc3)c3ccccc3)c2)nc(-n2c3c([2H])c([2H])c([2H])c([2H])c3c3c([2H])c([2H])c([2H])c([2H])c32)n1]
Ref. 6 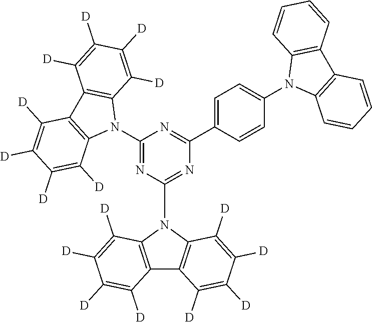 [SMILES: [2H]c1c([2H])c([2H])c2c(c1[2H])c1c([2H])c([2H])c([2H])c([2H])c1n2-c1nc(-c2ccc(-n3c4ccccc4c4ccccc43)cc2)nc(-n2c3c([2H])c([2H])c([2H])c([2H])c3c3c([2H])c([2H])c([2H])c([2H])c32)n1]
Ref. 7 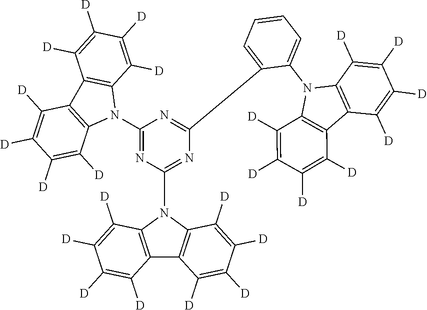 [SMILES: [2H]c1c([2H])c([2H])c2c(c1[2H])c1c([2H])c([2H])c([2H])c([2H])c1n2-c1nc(-c2ccccc2-n2c3c([2H])c([2H])c([2H])c([2H])c3c3c([2H])c([2H])c([2H])c([2H])c32)nc(-n2c3c([2H])c([2H])c([2H])c([2H])c3c3c([2H])c([2H])c([2H])c([2H])c32)n1]
Ref. 8 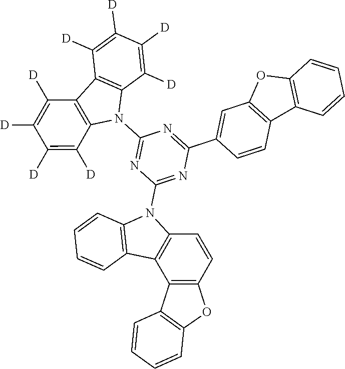 [SMILES: [2H]c1c([2H])c([2H])c2c(c1[2H])c1c([2H])c([2H])c([2H])c([2H])c1n2-c1nc(-c2ccc3c(c2)oc2ccccc23)nc(-n2c3ccccc3c3c4c(ccc32)oc2ccccc24)n1]
Ref. 9 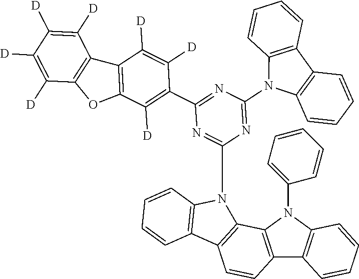 [SMILES: [2H]c1c([2H])c([2H])c2c(oc3c([2H])c(-c4nc(-n5c6ccccc6c6ccccc65)nc(-n5c6ccccc6c6ccc7c8ccccc8n(-c8ccccc8)c7c65)n4)c([2H])c([2H])c32)c1[2H]]
Ref. 10 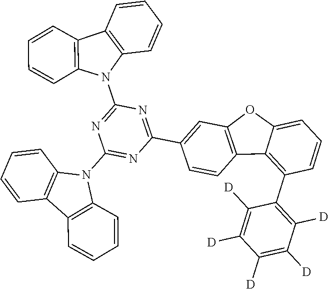 [SMILES: [2H]c1c([2H])c([2H])c(-c2cccc3oc4cc(-c5nc(-n6c7ccccc7c7ccccc76)nc(-n6c7ccccc7c7ccccc76)n5)ccc4c23)c([2H])c1[2H]]
Ref. 11 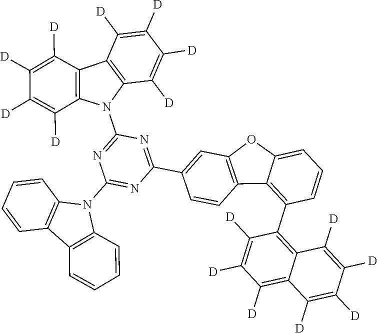 [SMILES: [2H]c1c([2H])c([2H])c2c(-c3cccc4oc5cc(-c6nc(-n7c8ccccc8c8ccccc87)nc(-n7c8c([2H])c([2H])c([2H])c([2H])c8c8c([2H])c([2H])c([2H])c([2H])c87)n6)ccc5c34)c([2H])c([2H])c([2H])c2c1[2H]]
Ref. 12 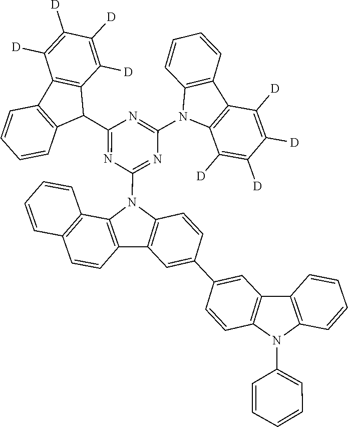 [SMILES: [2H]c1c([2H])c([2H])c2c(c1[2H])-c1ccccc1C2c1nc(-n2c3ccccc3c3c([2H])c([2H])c([2H])c([2H])c32)nc(-n2c3ccc(-c4ccc5c(c4)c4ccccc4n5-c4ccccc4)cc3c3ccc4ccccc4c32)n1]
Ref. 13 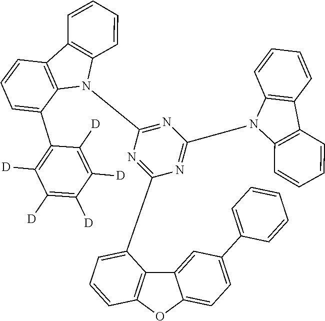 [SMILES: [2H]c1c([2H])c([2H])c(-c2cccc3c4ccccc4n(-c4nc(-c5cccc6oc7ccc(-c8ccccc8)cc7c56)nc(-n5c6ccccc6c6ccccc65)n4)c23)c([2H])c1[2H]]
Ref. 14

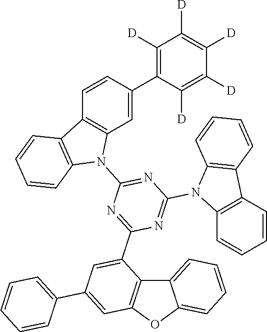 [SMILES: [2H]c1c([2H])c([2H])c(-c2ccc3c4ccccc4n(-c4nc(-c5cc(-c6ccccc6)cc6oc7ccccc7c56)nc(-n5c6ccccc6c6ccccc65)n4)c3c2)c([2H])c1[2H]]

As can be seen from the results in Table 9, the organic electroluminescence device using a light emitting layer material of the organic electroluminescence device of the present invention had a low driving voltage, an enhanced light emitting efficiency, and a significantly improved lifetime compared to those in Comparative Examples 1 to 14.

In general, compounds bonded with hydrogen and compounds substituted with deuterium exhibit a difference in thermodynamic behavior. The reason for this is that the mass of a deuterium atom is 2-fold higher than that of hydrogen, but due to the difference in the mass of atoms, deuterium is characterized by having even lower vibration energy. In addition, the bond length of carbon and deuterium is shorter than that of a bond with hydrogen, and a dissociation energy used to break the bond is also stronger than that of the bond with hydrogen. This is because the van der Waals radius of deuterium is smaller than that of hydrogen, and thus the extension amplitude of a bond between carbon and deuterium becomes even narrower.

The compounds of the present invention, substituted with deuterium, may have a higher light efficiency due to the weakening of the intermolecular van der Waals forces caused by the shorter carbon-deuterium bond length than the carbon-hydrogen bond length, compared to compounds not substituted with deuterium. Furthermore, zero point energy, that is, the energy of the ground state is reduced and the carbon-deuterium bond length is shortened, resulting in a reduction in molecular hardcore volume, and accordingly, the electrical polarizability may be reduced, and the thin film volume may be increased by weakening the intermolecular interaction. These characteristics induce an effect of lowering the crystallinity by creating the amorphous state of a thin film. In conclusion, deuterium substitution may be effective in improving the heat resistance of an OLED device, and accordingly, the service life and driving characteristics of the device may be improved. Furthermore, the effect of improving device characteristics by deuterium substitution is improved as the deuterium substitution rate in the molecule increases.

The compound of the present invention lowers the energy of the molecule by substituting carbazole which corresponds to the HOMO in the material with deuterium which has a higher molecular weight than hydrogen to reduce the change in vibrational frequency, and accordingly, a compound that increases the stability of the molecule was developed. Further, since the single bond dissociation energy of carbon and deuterium is higher than the single bond dissociation energy of carbon and hydrogen, it can be confirmed that the service life of the device is improved as the thermal stability of the molecule is enhanced.

When deuterium is substituted, the intermolecular distance is close, so that charge mobility increases, and accordingly, it can be confirmed that the service life of the device is improved. Furthermore, it can be confirmed that the device characteristics are improved, as the deuterium substitution rate in the molecule increases.

In Comparative Examples 1 to 4, which are not substituted with deuterium, the electron mobility in the molecule is faster than hole mobility, and accordingly, it is determined that the recombination zone is biased towards the HTL side, resulting in a decrease in the efficiency and service life of the device.

In contrast, in Comparative Examples 5 to 8 and 12, as strong donating units were added in the molecule, strong HT characteristics are obtained, and accordingly, it could be confirmed that electrons are not effectively stabilized, resulting in a reduction in service life.

In contrast, in Comparative Examples 9 to 11, as D substitution is performed on the substituent corresponding to the LUMO, electron mobility is rather faster, and accordingly, it can be confirmed that Comparative Example 10 has a further deterioration in device performance than Comparative Example 3.

As shown in Examples 23 to 28, it can be confirmed that the device characteristics are improved as the deuterium substitution rate in the molecule increases. In contrast, in Comparative Examples 13 to 14, the phenyl group corresponding to the HOMO was substituted with deuterium, but deuterium was not included in carbazole, and the proportion of deuterium in the HOMO was small, making it difficult to confirm the effect.

This suggests that even though they have similar structures, the characteristics of the compounds may vary depending on the substitution with deuterium.

### <Experimental Example 2>—Manufacture of Organic Light Emitting Device

A glass substrate thinly coated with indium tin oxide (ITO) having a thickness of 1,500 Å was ultrasonically washed with distilled water. When the washing with distilled water was finished, the glass substrate was ultrasonically washed with a solvent such as acetone, methanol, and isopropyl alcohol, dried and then subjected to ultraviolet ozone (UVO) treatment for 5 minutes using UV in an ultraviolet (UV) washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4′,4″-tris[2-naphthyl(phenyl)amino]triphenylamine (2-TNATA) and the hole transport layer N,N′-di(1-naphthyl)-N,N′-diphenyl-(1,1′-biphenyl)-4,4′-diamine (NPB) were formed on the ITO transparent electrode (positive electrode).

A light emitting layer was thermally vacuum deposited thereon as follows. Pre-mixing was performed by pre-mixing one type of compound of Chemical Formula 1 and one type of compound of Chemical Formula 2 or 3 as hosts as described in the following Table 10, and then the light emitting layer was deposited to have a thickness of 400 Å from one common container, and a green phosphorescent dopant was deposited by doping the host with a green phosphorescent dopant [Ir(ppy)₃] in an amount of 7% of the deposition thickness of the light emitting layer. Thereafter, bathocuproine (BCP) as a hole blocking layer was deposited to have a thickness of 60 Å, and Alq3 as an electron transport layer was deposited to have a thickness of 200 Å thereon. Finally, lithium fluoride (LiF) was deposited to have a thickness of 10 Å on the electron transport layer to form an electron injection layer, and then an aluminum (Al) negative electrode was deposited to have a thickness of 1,200 Å on the electron injection layer to form a negative electrode, thereby manufacturing an organic electroluminescence device.

Meanwhile, all the organic compounds required for manufacturing an OLED device were subjected to vacuum sublimed purification under 10⁻⁸ to 10⁻⁶ torr for each material, and used for the manufacture of the OLED.

For the organic electroluminescence device manufactured as described above, electroluminescence (EL) characteristics were measured by M7000 manufactured by McScience Inc., and based on the measurement result thereof, T₉₀ was measured by a service life measurement device (M6000) manufactured by McScience Inc., when the reference luminance was 6,000 cd/m2.

The results of measuring the driving voltage, light emitting efficiency, color coordinate (CIE) and service life of the organic light emitting device manufactured according to the present invention are shown in the following Table 10.

TABLE 10

| | Light | | Driving | | | Service |
|---|---|---|---|---|---|---|
| | emitting | | voltage | Efficiency | | life |
| | compound | Ratio | (V) | (cd/A) | EL color | (T₉₀) |
| Comparative | Ref. 1:2-3 | 1:1 | 4.39 | 56.6 | Green | 117 |
| Example 15 | | | | | | |
| Comparative | | 1:2 | 4.57 | 57.8 | | 129 |
| Example 16 | | | | | | |
| Comparative | | 1:3 | 4.61 | 60.61 | | 138 |
| Example 17 | | | | | | |
| Comparative | Ref. 5:3-4 | 1:1 | 5.27 | 52.3 | | 72 |
| Example 18 | | | | | | |
| Comparative | | 1:2 | 5.32 | 54.6 | | 77 |
| Example 19 | | | | | | |
| Comparative | | 1:3 | 5.40 | 57.2 | | 81 |
| Example 20 | | | | | | |
| Comparative | Ref. 8:2-100 | 1:1 | 4.48 | 47.7 | | 84 |
| Example 21 | | | | | | |
| Comparative | | 1:2 | 4.52 | 50.9 | | 89 |
| Example 22 | | | | | | |
| Comparative | | 1:3 | 4.59 | 52.3 | | 95 |
| Example 23 | | | | | | |
| Example 31 | 1-40:2-3 | 1:1 | 3.78 | 77.4 | | 274 |
| Example 32 | | 1:2 | 3.85 | 78.2 | | 288 |
| Example 33 | | 1:3 | 4.02 | 82.9 | | 305 |
| Example 34 | 1-40:3-4 | 1:1 | 3.55 | 74.9 | | 294 |
| Example 35 | | 1:2 | 3.67 | 75.1 | | 309 |
| Example 36 | | 1:3 | 3.88 | 76.4 | | 328 |
| Example 37 | 1-78:2-83 | 1:1 | 3.80 | 76.9 | | 356 |
| Example 38 | | 1:2 | 3.84 | 77.3 | | 381 |
| Example 39 | | 1:3 | 4.05 | 80.4 | | 419 |
| Example 40 | 1-177:2-100 | 1:1 | 3.71 | 80.3 | | 367 |
| Example 41 | | 1:2 | 3.81 | 82.7 | | 382 |
| Example 42 | | 1:3 | 3.98 | 84.1 | | 407 |
| Example 43 | 1-208:3-1 | 1:1 | 3.52 | 85.5 | | 284 |
| Example 44 | | 1:2 | 3.67 | 87.8 | | 298 |
| Example 45 | | 1:3 | 3.72 | 90.1 | | 311 |
| Example 46 | 1-211:3-4 | 1:1 | 3.55 | 87.1 | | 287 |
| Example 47 | | 1:2 | 3.59 | 88.4 | | 300 |
| Example 48 | | 1:3 | 3.68 | 89.9 | | 308 |
| Example 49 | 1-298:3-5 | 1:1 | 3.62 | 80.9 | | 311 |
| Example 50 | | 1:2 | 3.67 | 82.6 | | 327 |
| Example 51 | | 1:3 | 3.72 | 84.3 | | 339 |
| Example 52 | 1-301:3-32 | 1:1 | 4.01 | 77.1 | | 302 |
| Example 53 | | 1:2 | 4.09 | 80.9 | | 317 |
| Example 54 | | 1:3 | 4.18 | 82.4 | | 334 |
| Example 55 | 1-400:2-82 | 1:1 | 3.66 | 76.5 | | 422 |
| Example 56 | | 1:2 | 3.74 | 78.9 | | 435 |
| Example 57 | | 1:3 | 3.85 | 80.1 | | 447 |
| Example 58 | 1-400:3-77 | 1:1 | 3.49 | 72.9 | | 455 |
| Example 59 | | 1:2 | 3.54 | 73.8 | | 469 |
| Example 60 | | 1:3 | 3.67 | 75.2 | | 486 |
| Example 61 | 1-404:2-83 | 1:1 | 3.54 | 74.1 | | 389 |
| Example 62 | | 1:2 | 3.63 | 75.4 | | 409 |
| Example 63 | | 1:3 | 3.79 | 77.3 | | 421 |
| Example 64 | 1-404:3-77 | 1:1 | 3.44 | 69.9 | | 419 |
| Example 65 | | 1:2 | 3.52 | 71.9 | | 433 |
| Example 66 | | 1:3 | 3.58 | 74.1 | | 458 |

Referring to the results of Table 10, better efficiency and service life effects are exhibited when the compound of Chemical Formula 1 (n type) and the compound of Chemical Formula 2 or 3 are simultaneously included. From this result, it can be expected that an exciplex phenomenon will occur when both compounds are included.

The exciplex phenomenon is a phenomenon in which energy with a magnitude of the HOMO level of a donor (p-host) and the LUMO level of an acceptor (n-host) is released due to an electron exchange between two molecules. When the exciplex phenomenon between two molecules occurs, a reverse intersystem crossing (RISC) occurs, and the internal quantum efficiency of fluorescence can be increased to 100% due to the RISC. When a donor with a good hole transport capacity (p-host) and an acceptor with a good electron transport capacity (n-host) are used as hosts for the light emitting layer, holes are injected into the p-host and electrons are injected into the n-host, so that the driving voltage can be lowered, which can help to improve the service life. In the present invention, it could be confirmed that excellent device characteristics are exhibited when the compound of Chemical Formula 2 or 3 serving as a donor role and the compound of Chemical Formula 1 serving as an acceptor role are used as hosts of the light emitting layer.

1. A heterocyclic compound represented by the following Chemical Formula 1: 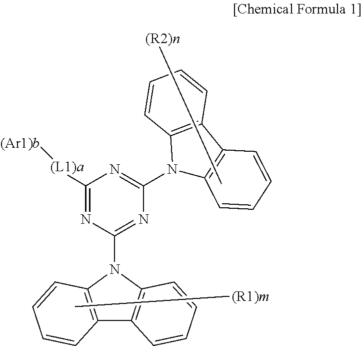
wherein, in Chemical Formula 1,
R1 and R2 are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more adjacent groups are bonded to each other to form a substituted or unsubstituted C6 to C60 aromatic hydrocarbon ring or a substituted or unsubstituted C2 to C60 hetero ring, m and n are each an integer from 1 to 8, and when m is 2 or higher, R1's are the same as or different from each other, and when n is 2 or higher, R2's are the same as or different from each other,
L1 is a direct bond; a substituted or unsubstituted C6 to C60 arylene group; or a substituted or unsubstituted C2 to C60 heteroarylene group, a is an integer from 1 to 4, and when a is 2 or higher, L1's are the same as or different from each other,
Ar1 is a substituted or unsubstituted C1 to C60 alkyl group; a substituted or unsubstituted C6 to C60 aryl group; or a substituted or unsubstituted C2 to C60 heteroaryl group comprising O or S,
b is an integer from 1 to 4, and when b is 2 or higher, Ar1's are the same as or different from each other, and
a deuterium content of 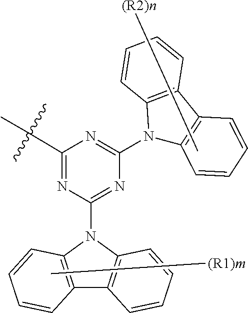
of Chemical Formula 1 is 20% or more and 100% or less.

2. The heterocyclic compound of claim 1, wherein 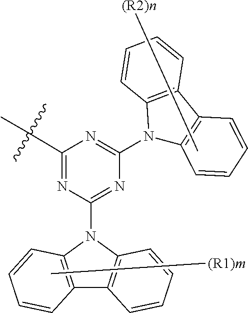
of Chemical Formula 1 is represented by any one of the following Chemical Formulae 4 to 17: 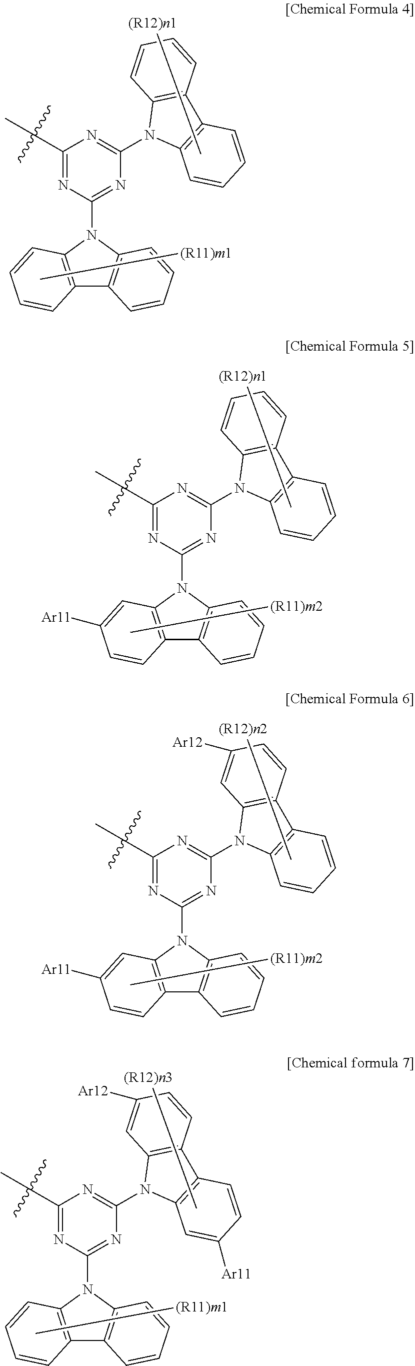 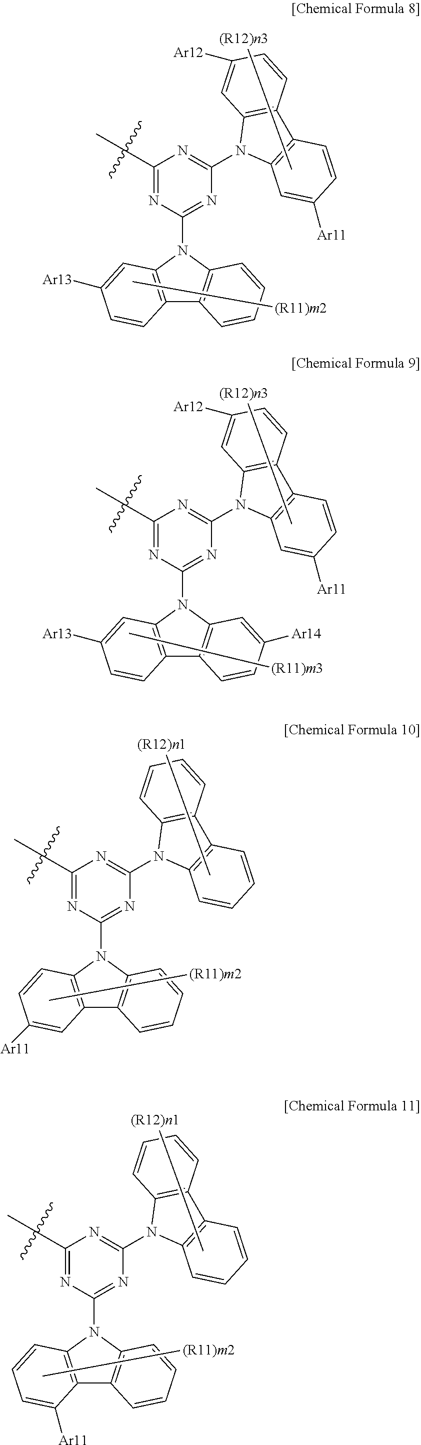 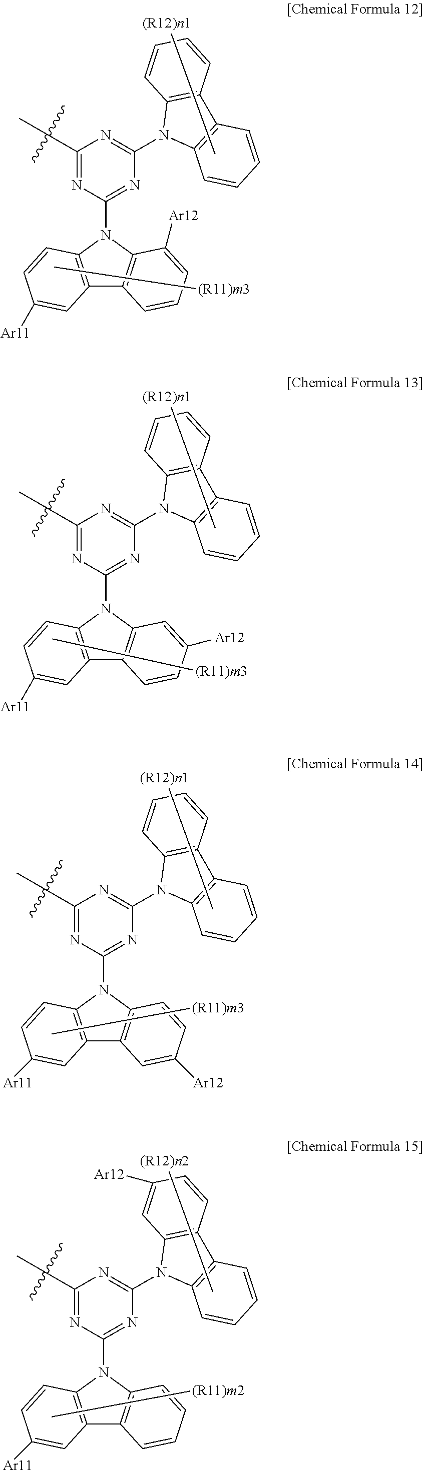 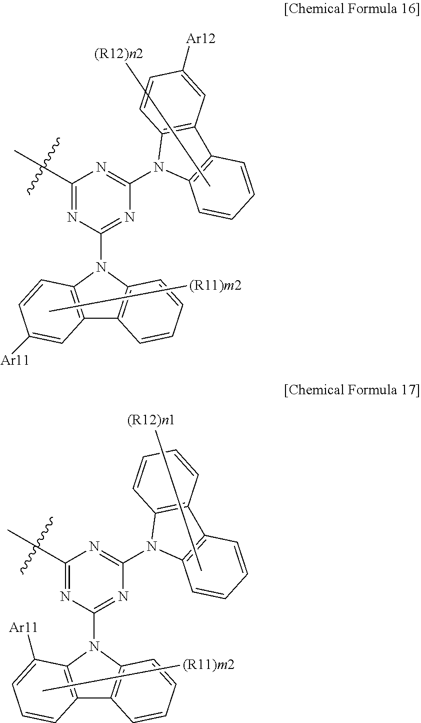
in Chemical Formulae 4 to 17,
R11 and R12 are the same as or different from each other, and are each independently hydrogen or deuterium,
Ar11 to Ar14 are the same as or different from each other, and are each independently a substituted or unsubstituted C6 to C60 aryl group; or a substituted or unsubstituted C2 to C60 heteroaryl group,
m1 and n1 are an integer from 0 to 8,
m2 and n2 are an integer from 0 to 7,
m3 and n3 are an integer from 0 to 6, and
when m1, m2, m3, n1, n2, and n3 are each 2 or higher, substituents in the parenthesis are the same as or different from each other.

3. The heterocyclic compound of claim 1, wherein Chemical Formula 1 is represented by each of the following Structural Formulae A and B, a deuterium content of the following Structural Formula A is 10% or less, and a deuterium content of the following Structural Formula B is 20% or more and 100% or less: 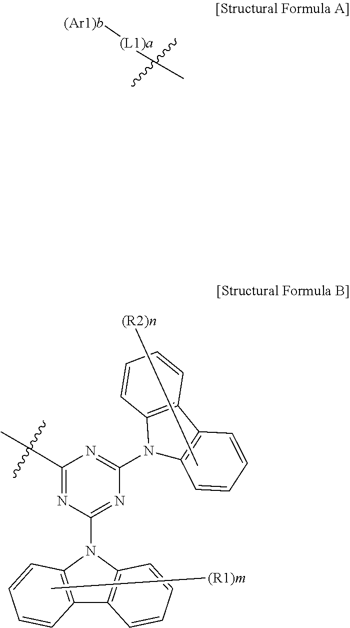
in Structural Formulae A and B, a definition of each substituent is the same as a definition in Chemical Formula 1.

4. The heterocyclic compound of claim 1, wherein Ar1 is a substituted or unsubstituted phenyl group; a substituted or unsubstituted biphenyl group; a substituted or unsubstituted terphenyl group; a substituted or unsubstituted quaterphenyl group; a substituted or unsubstituted quinquephenyl group; a substituted or unsubstituted triphenylenyl group; a substituted or unsubstituted dibenzofuran group; a substituted or unsubstituted dibenzothiophene group; a substituted or unsubstituted dimethylfluorenyl group; a substituted or unsubstituted diphenylfluorenyl group; or a substituted or unsubstituted spirobifluorenyl group.

5. The heterocyclic compound of claim 1, wherein Chemical Formula 1 is represented by any one of the following compounds:

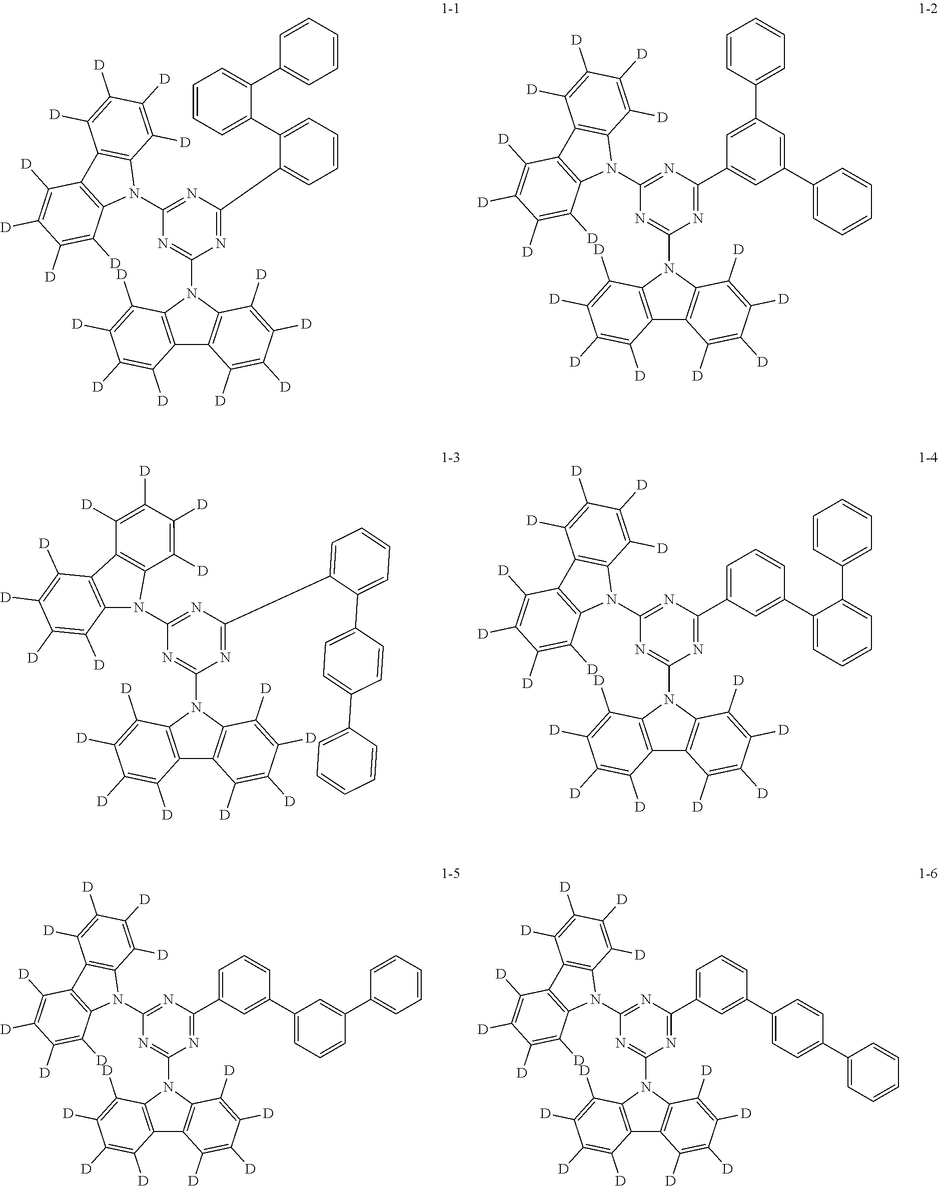

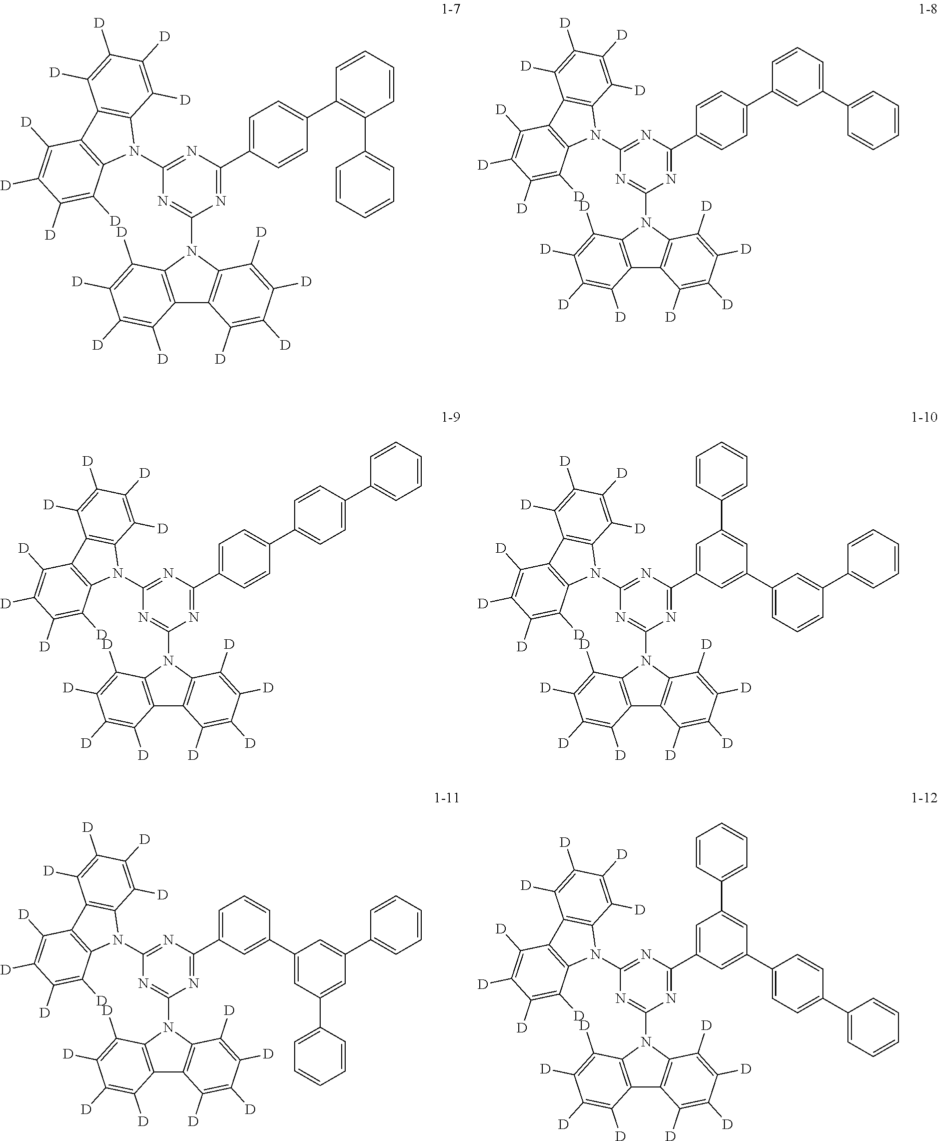

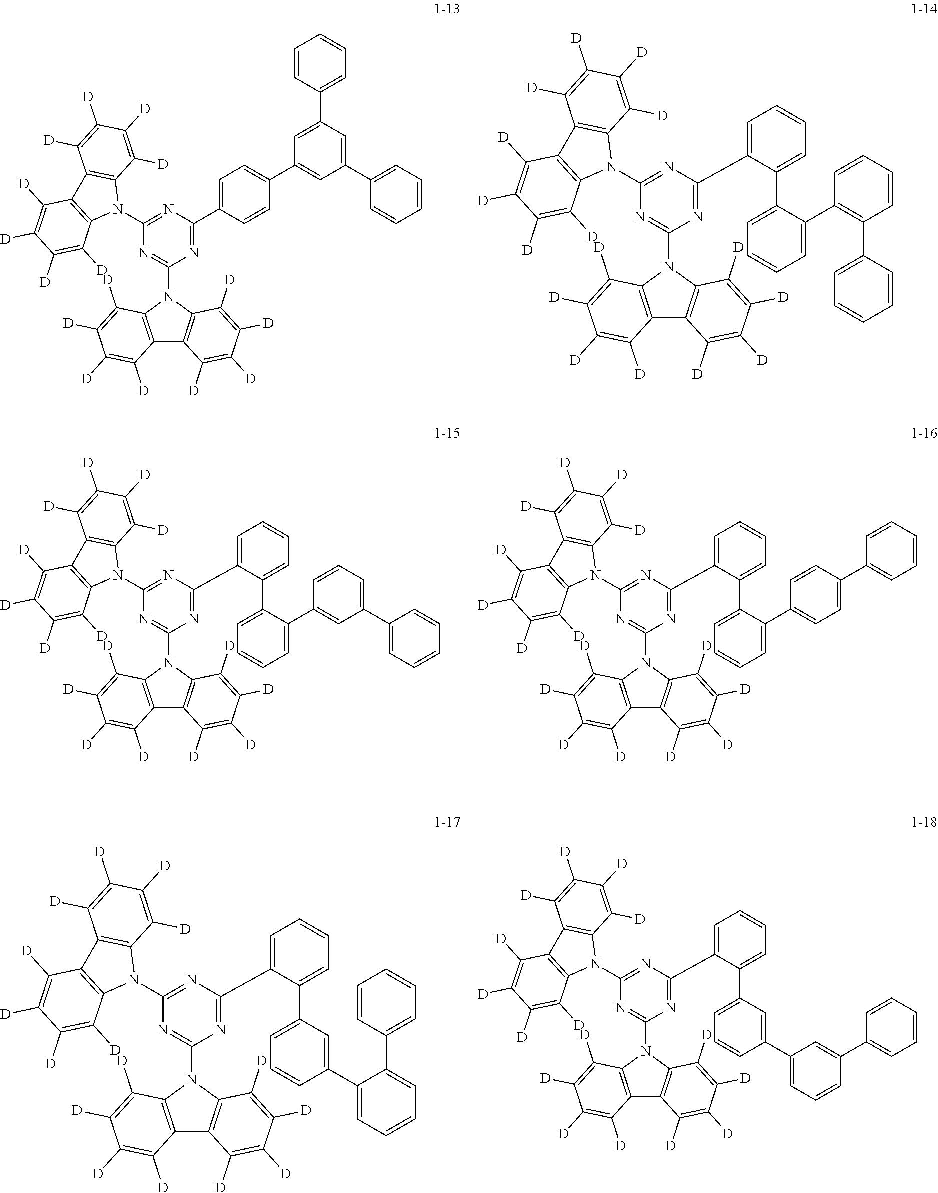

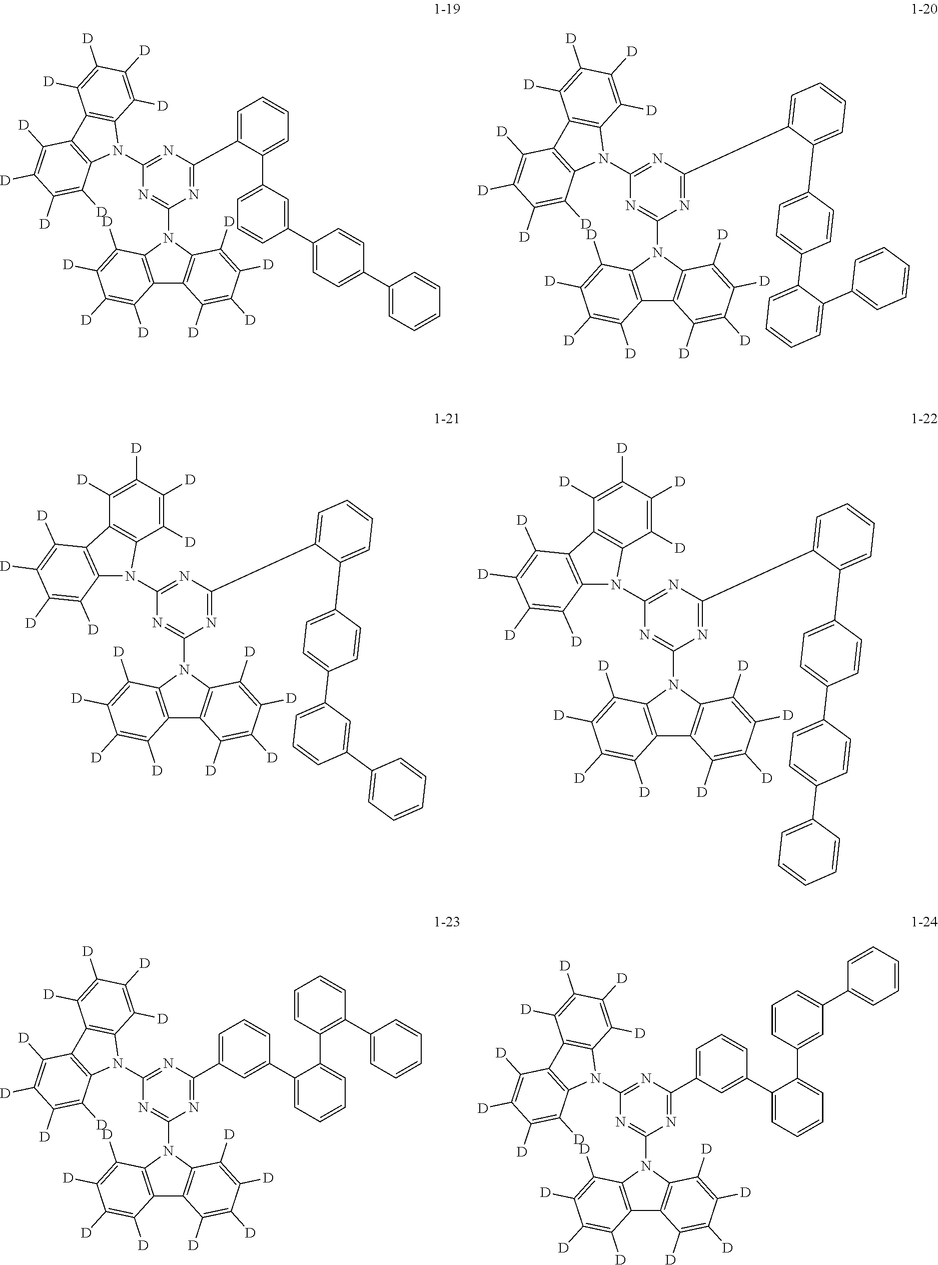

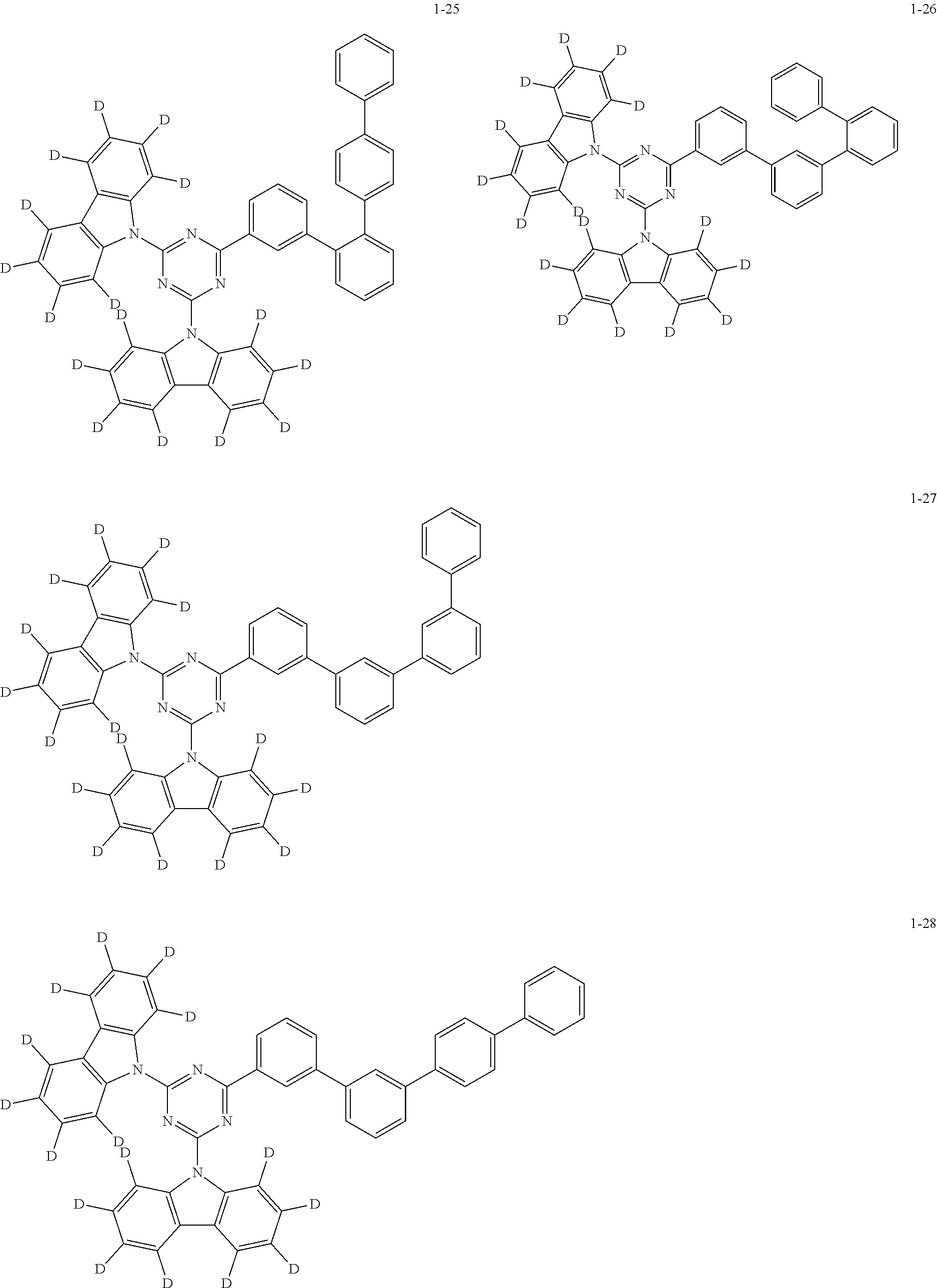

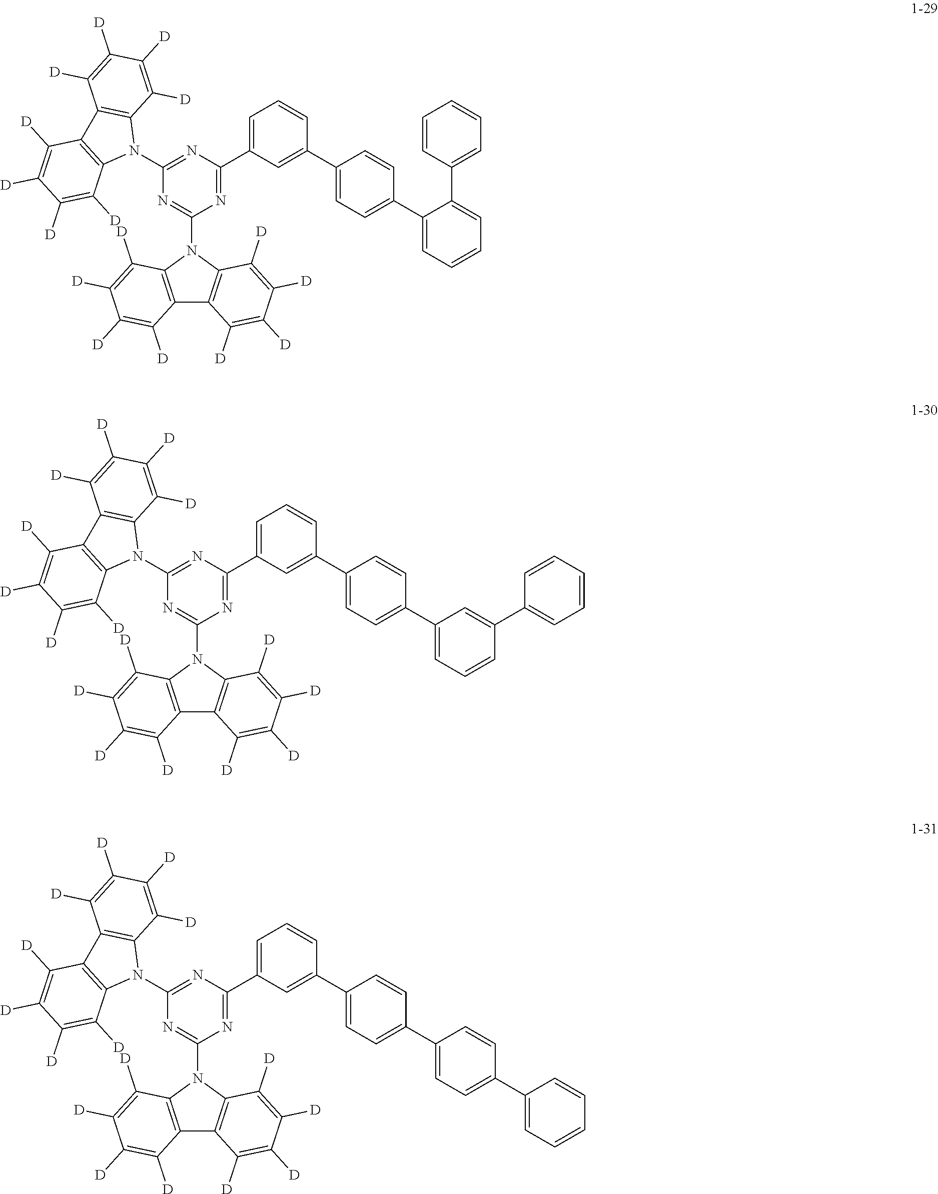

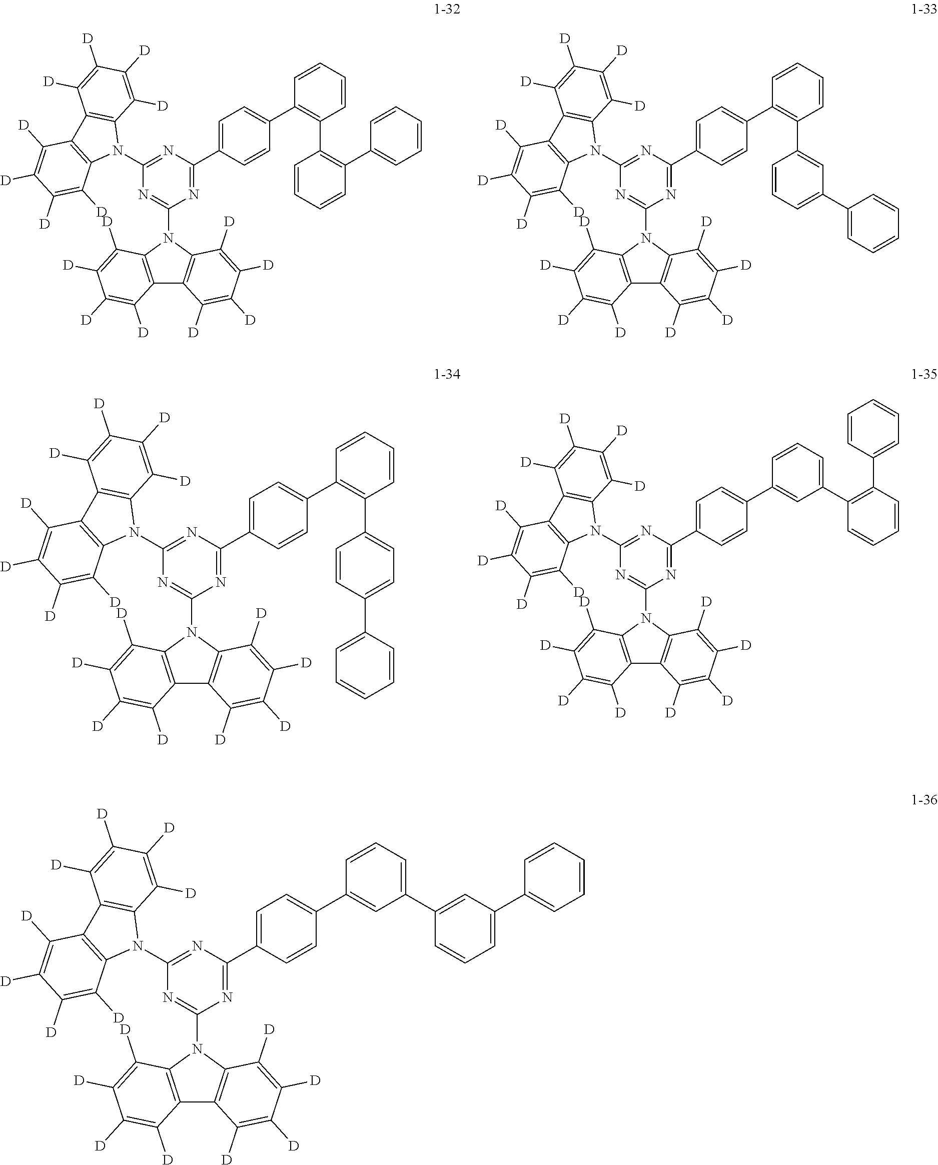

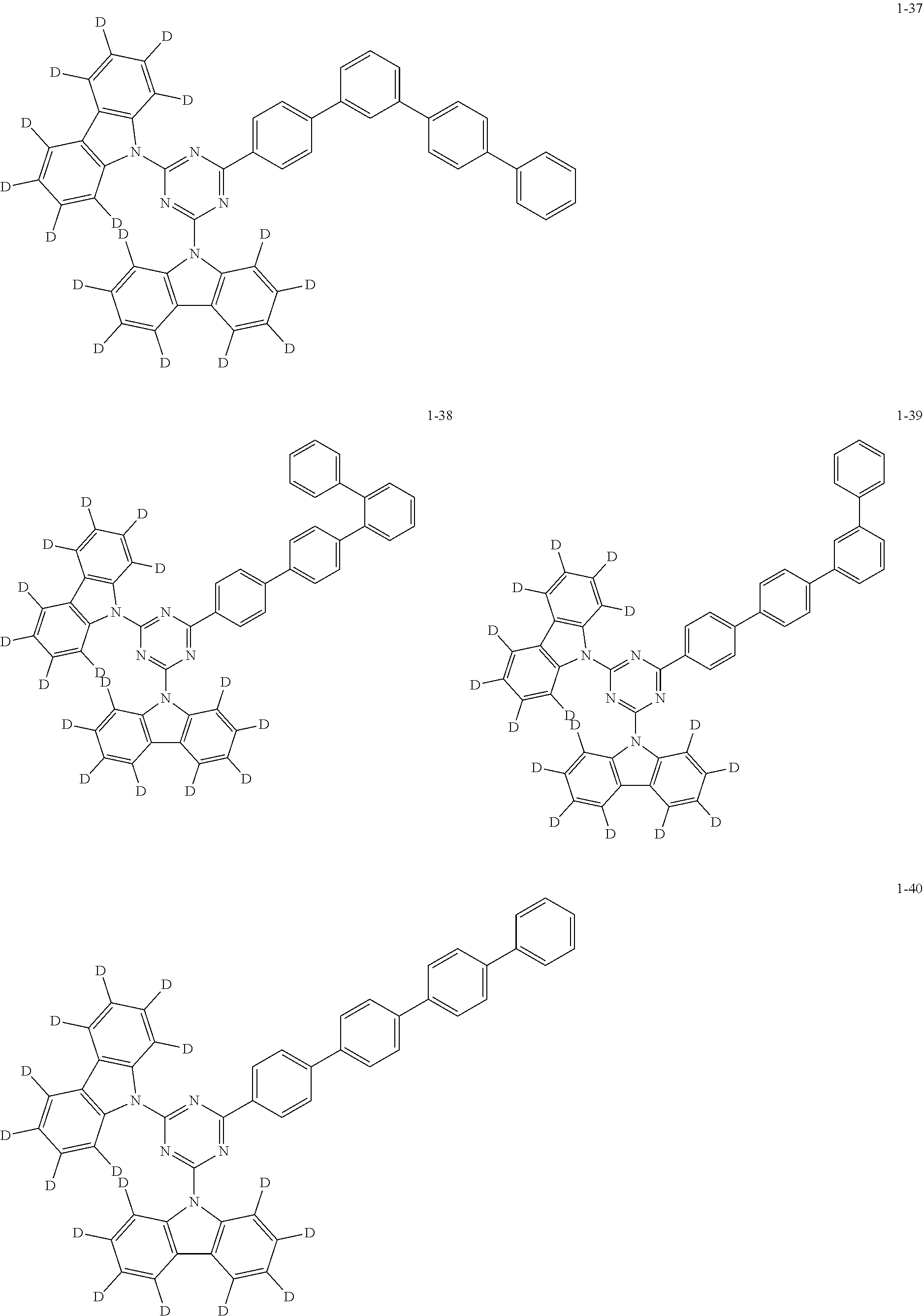

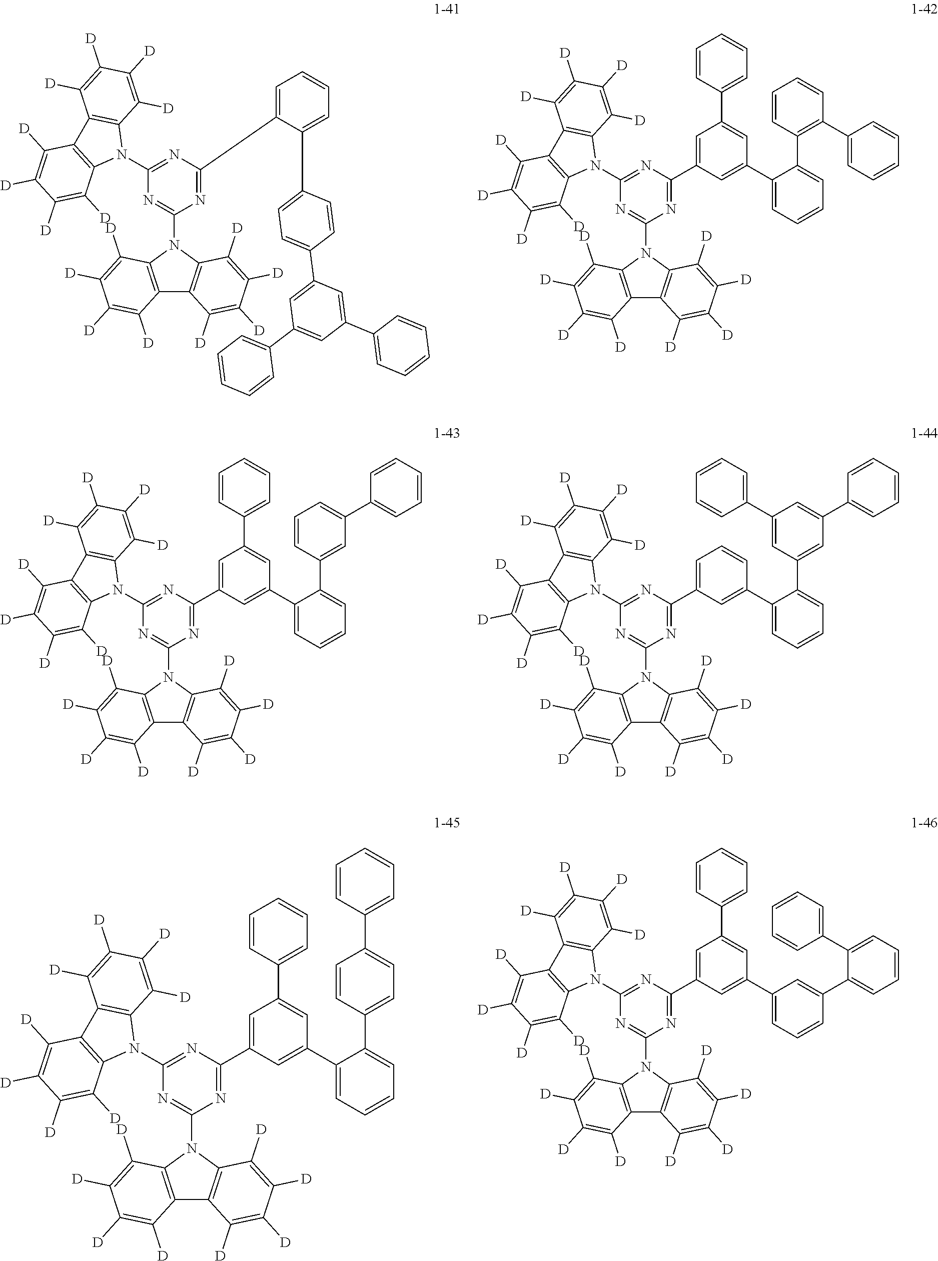

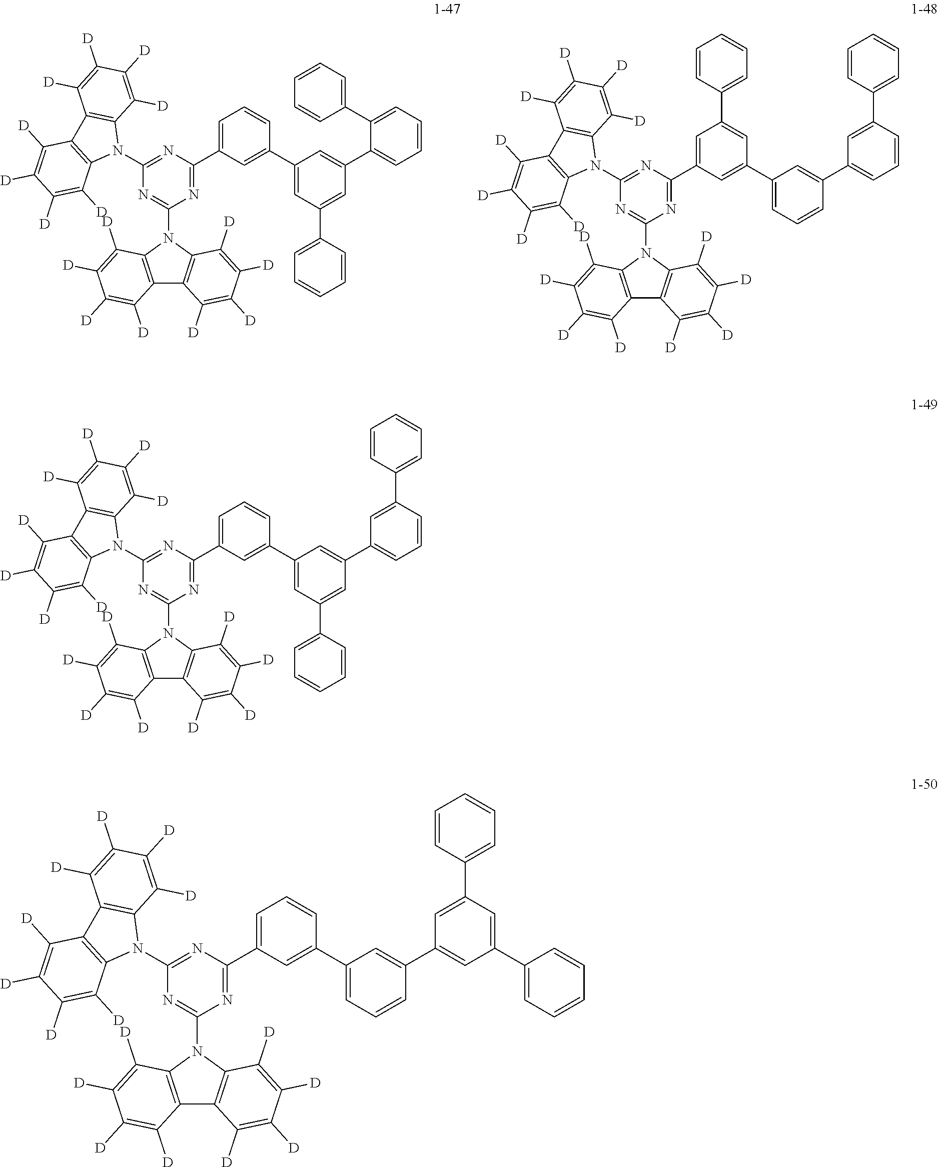

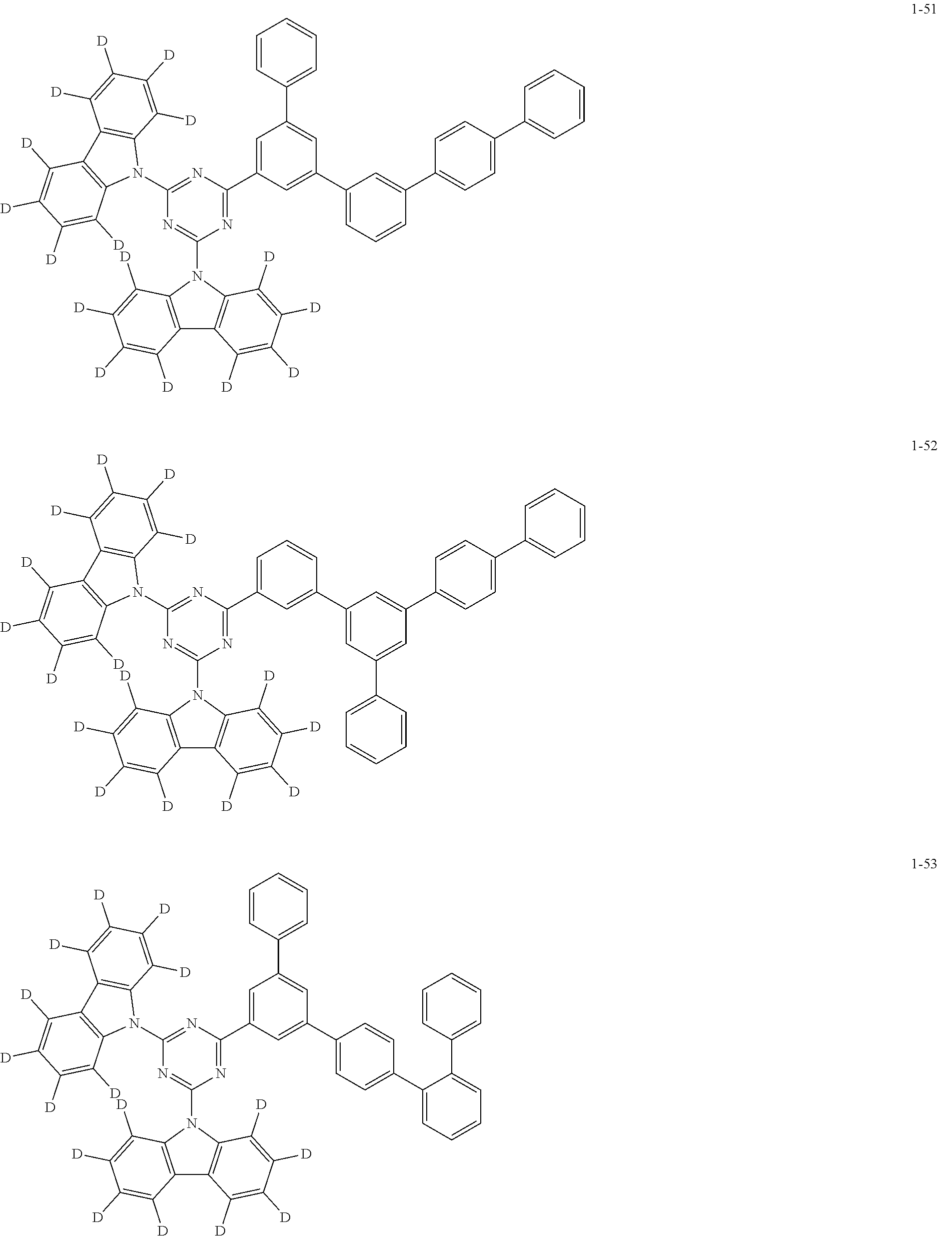

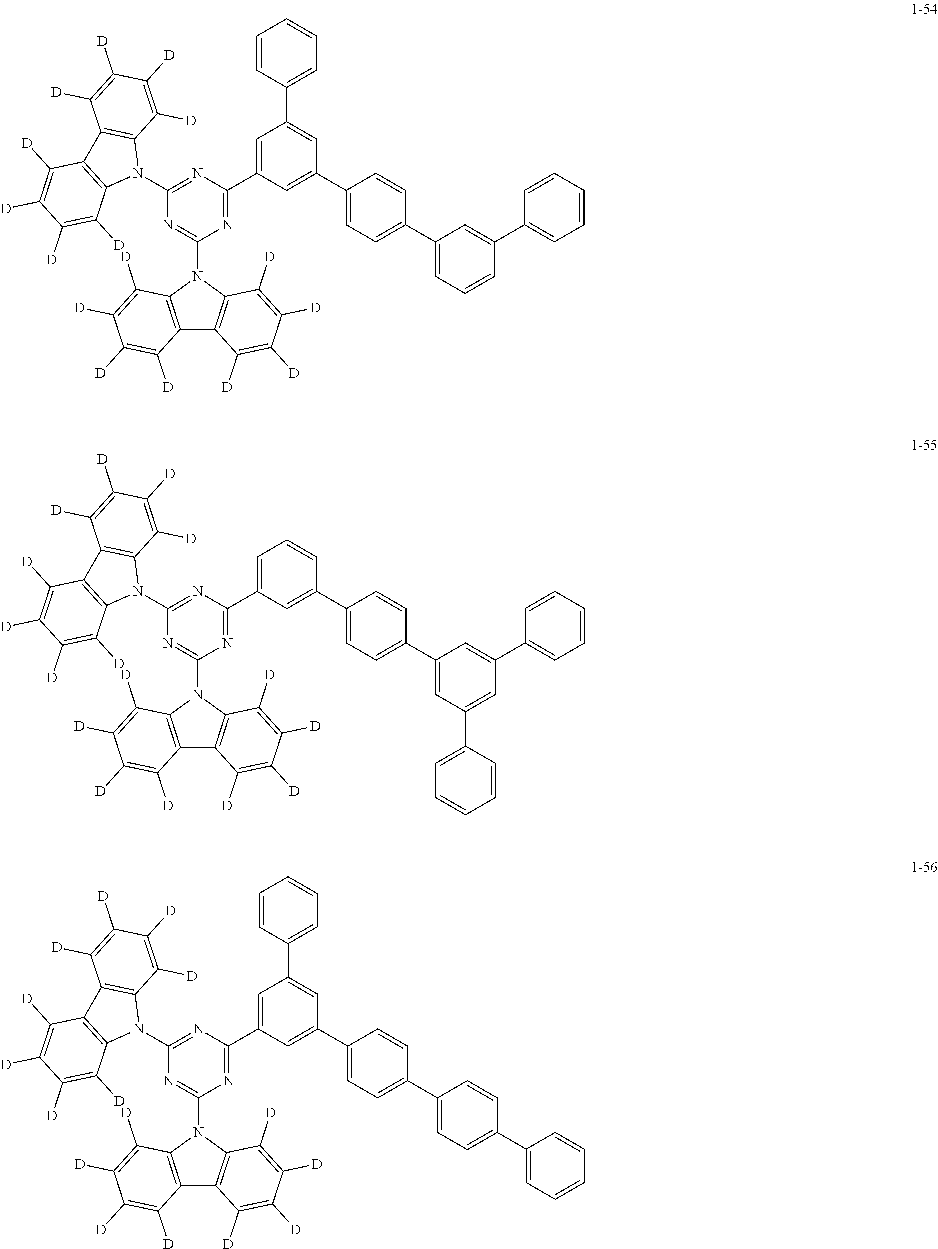

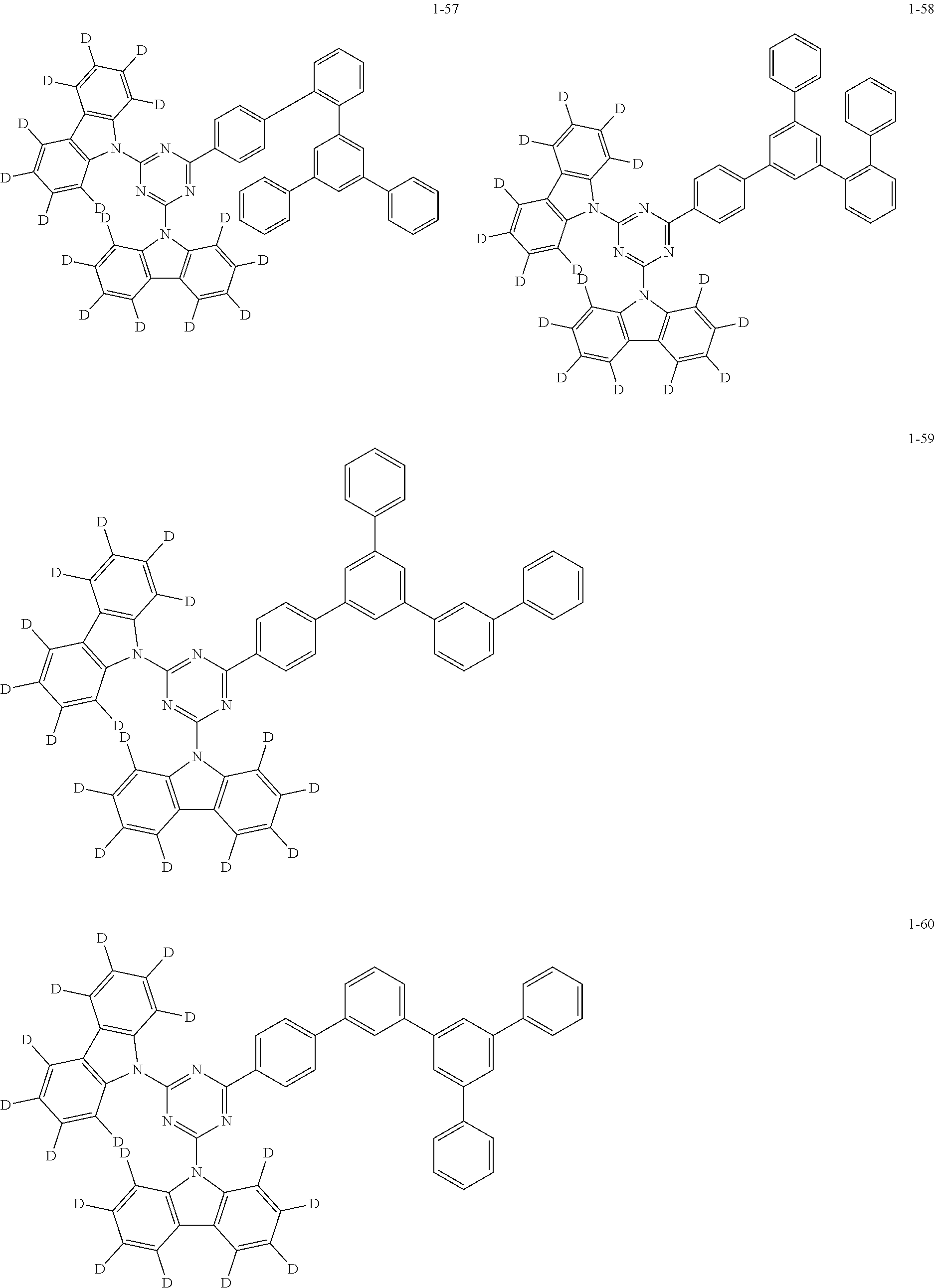

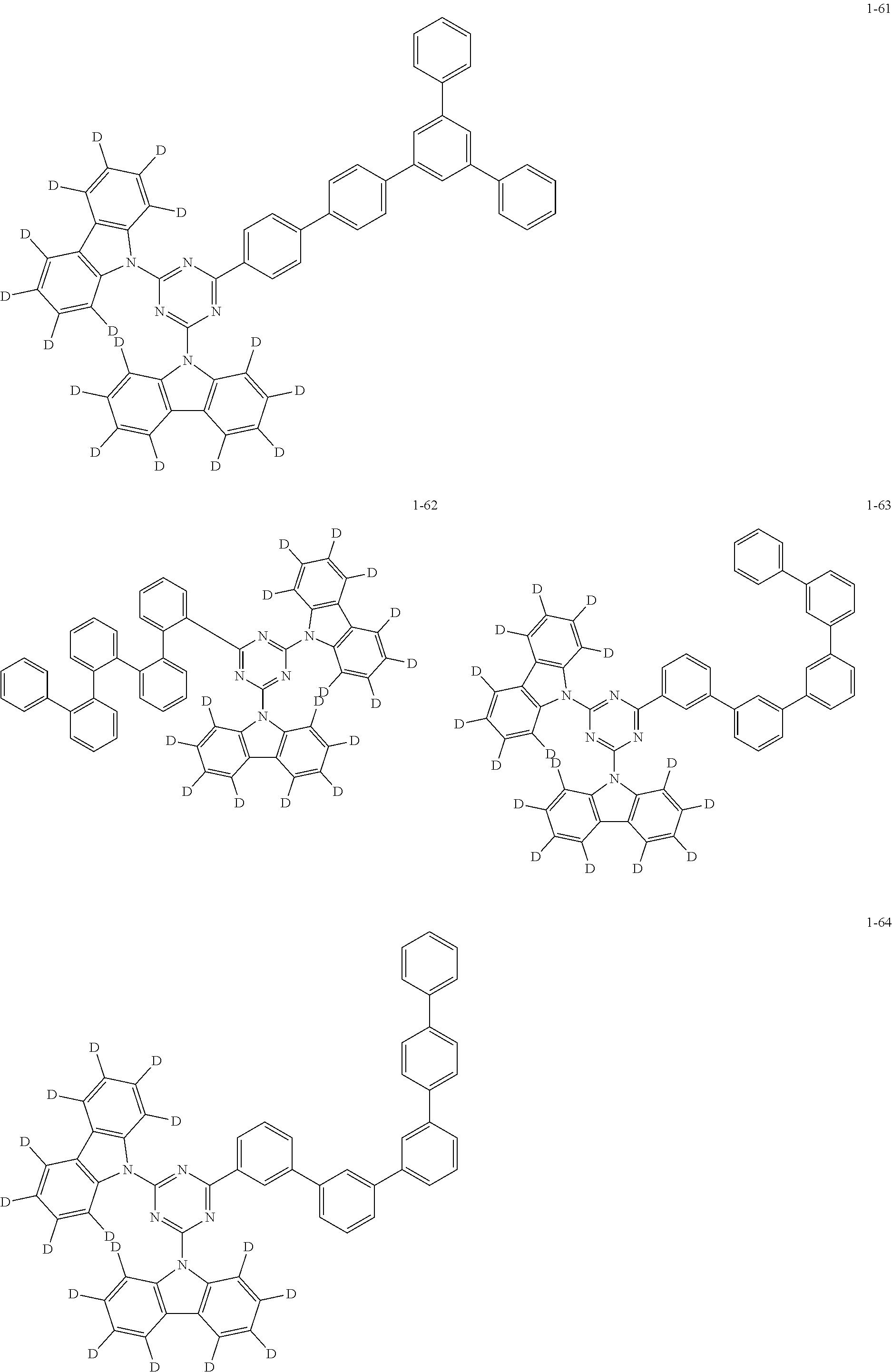

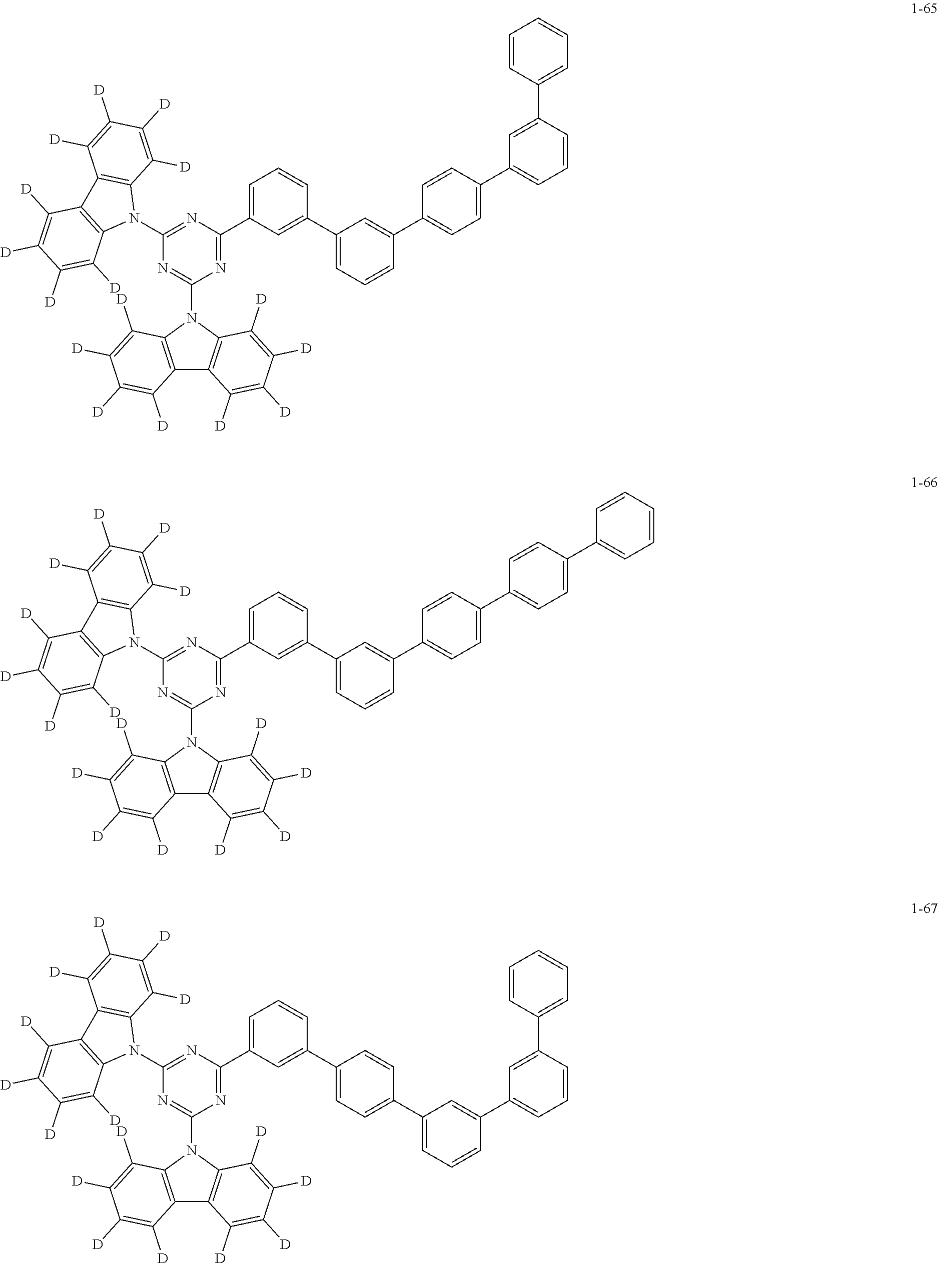

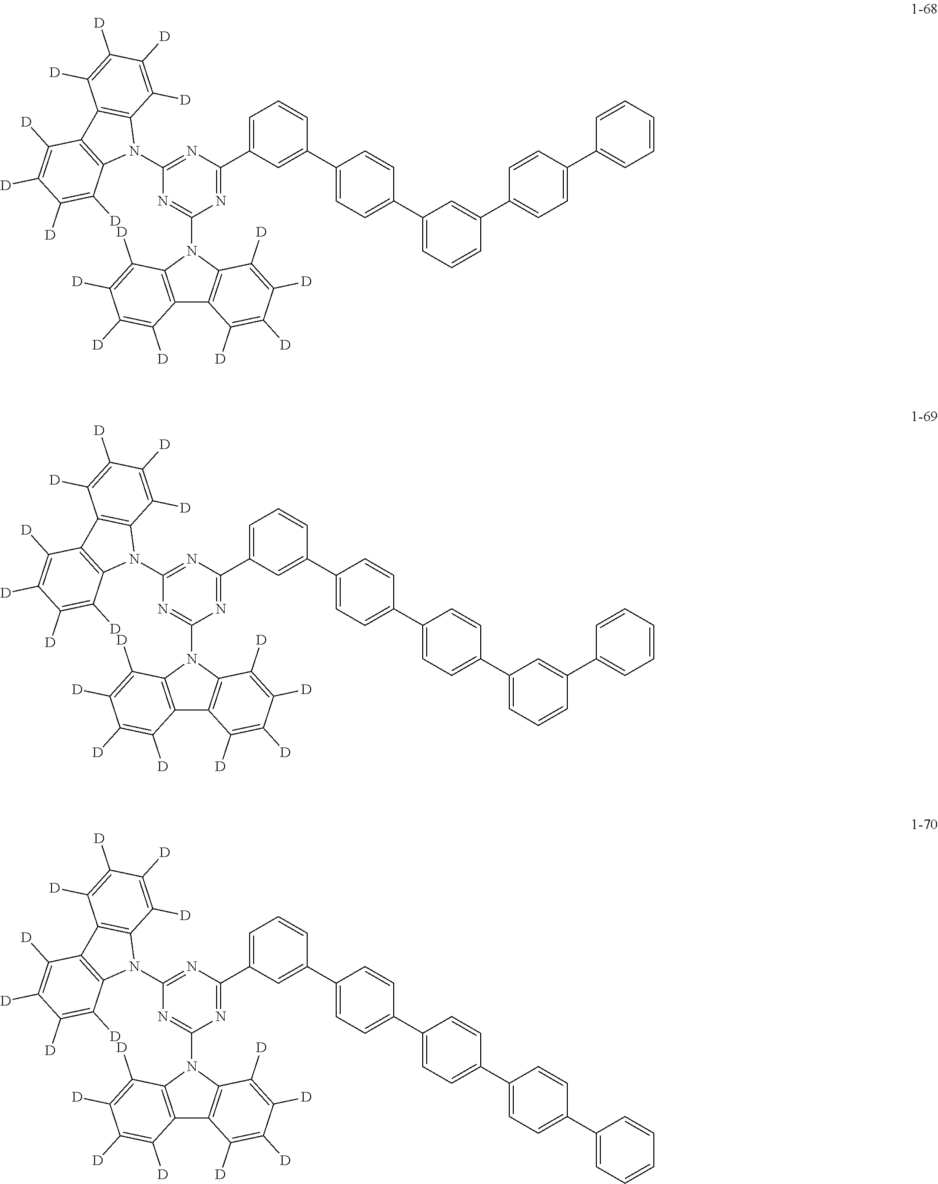

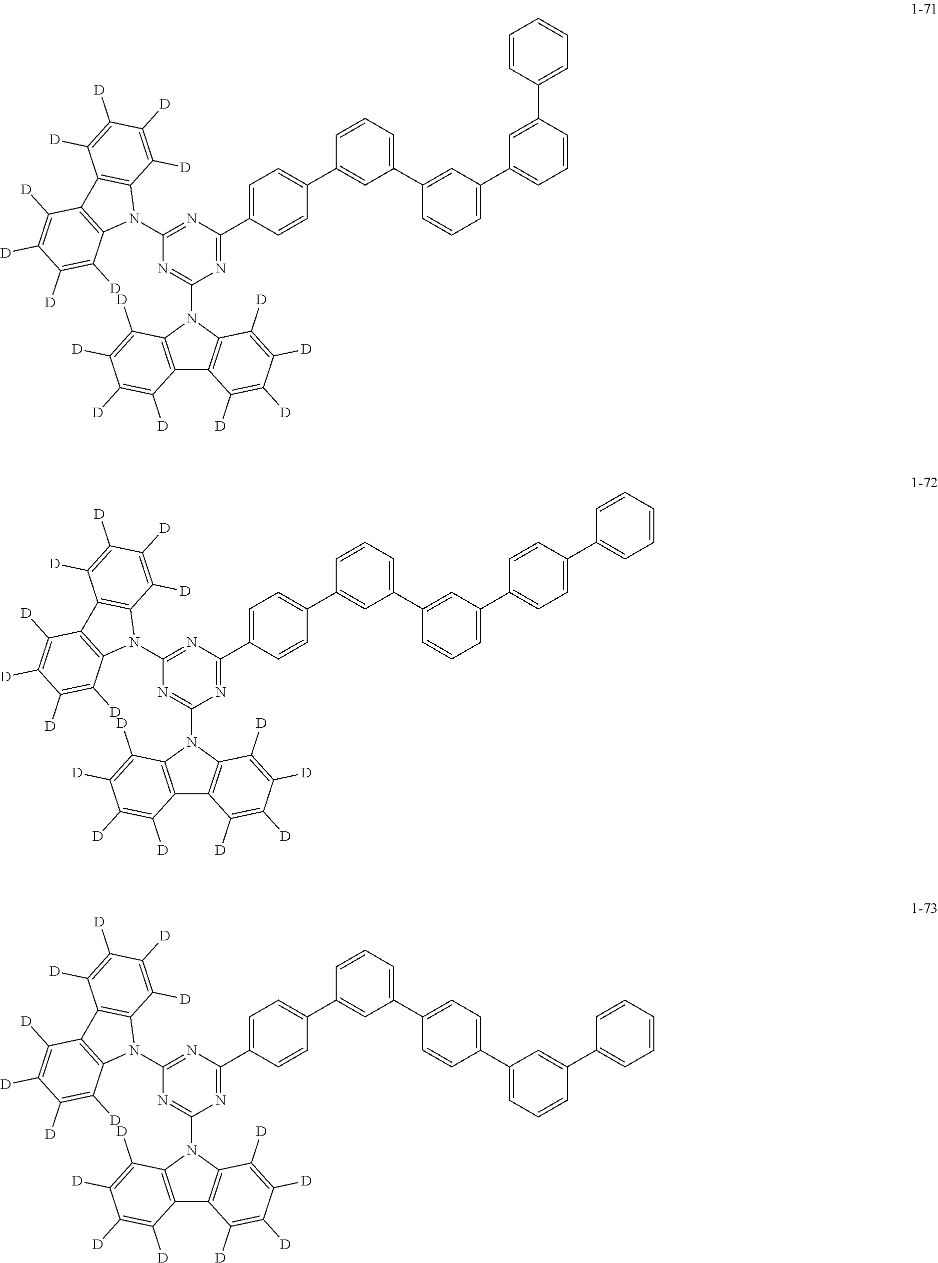

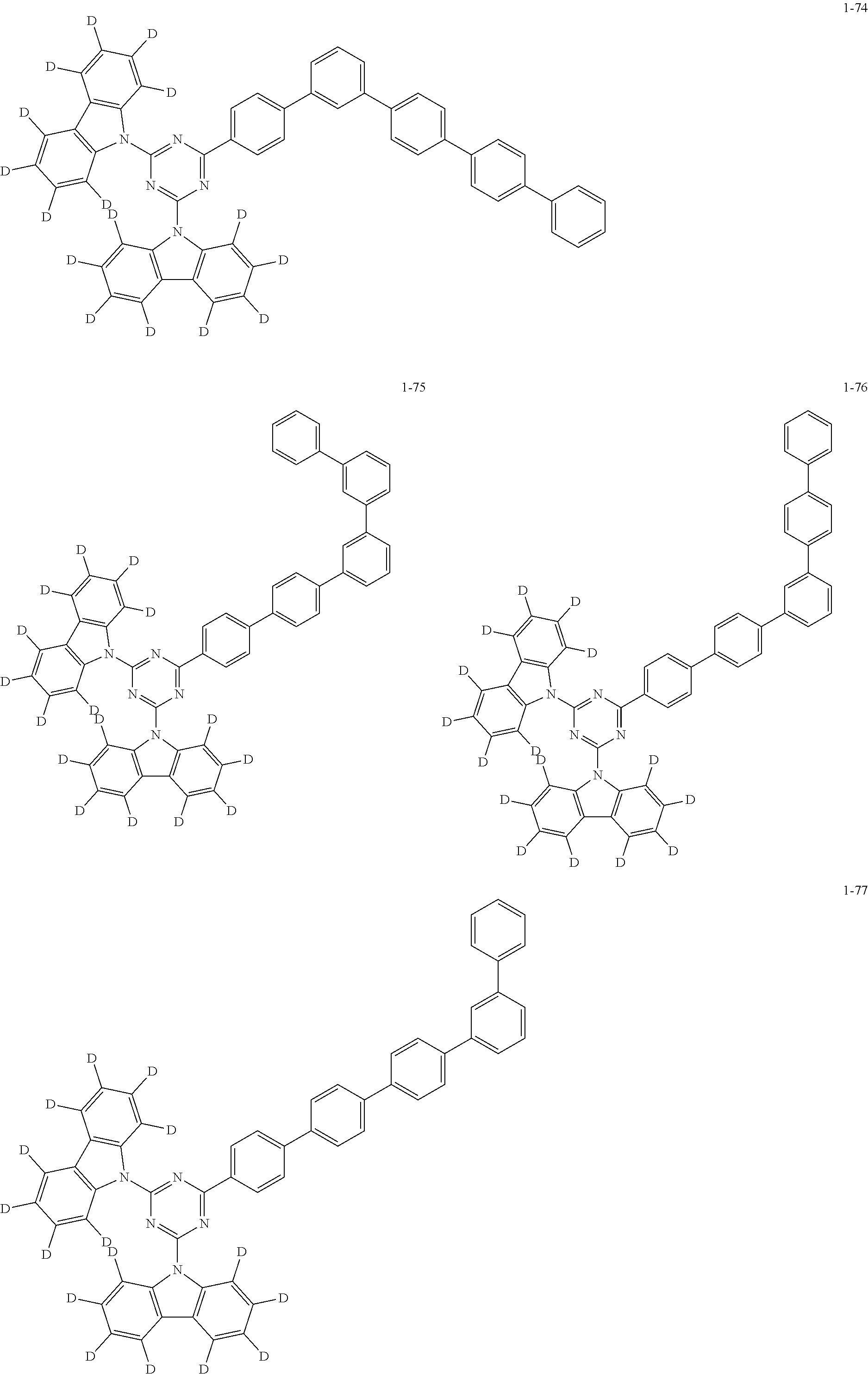

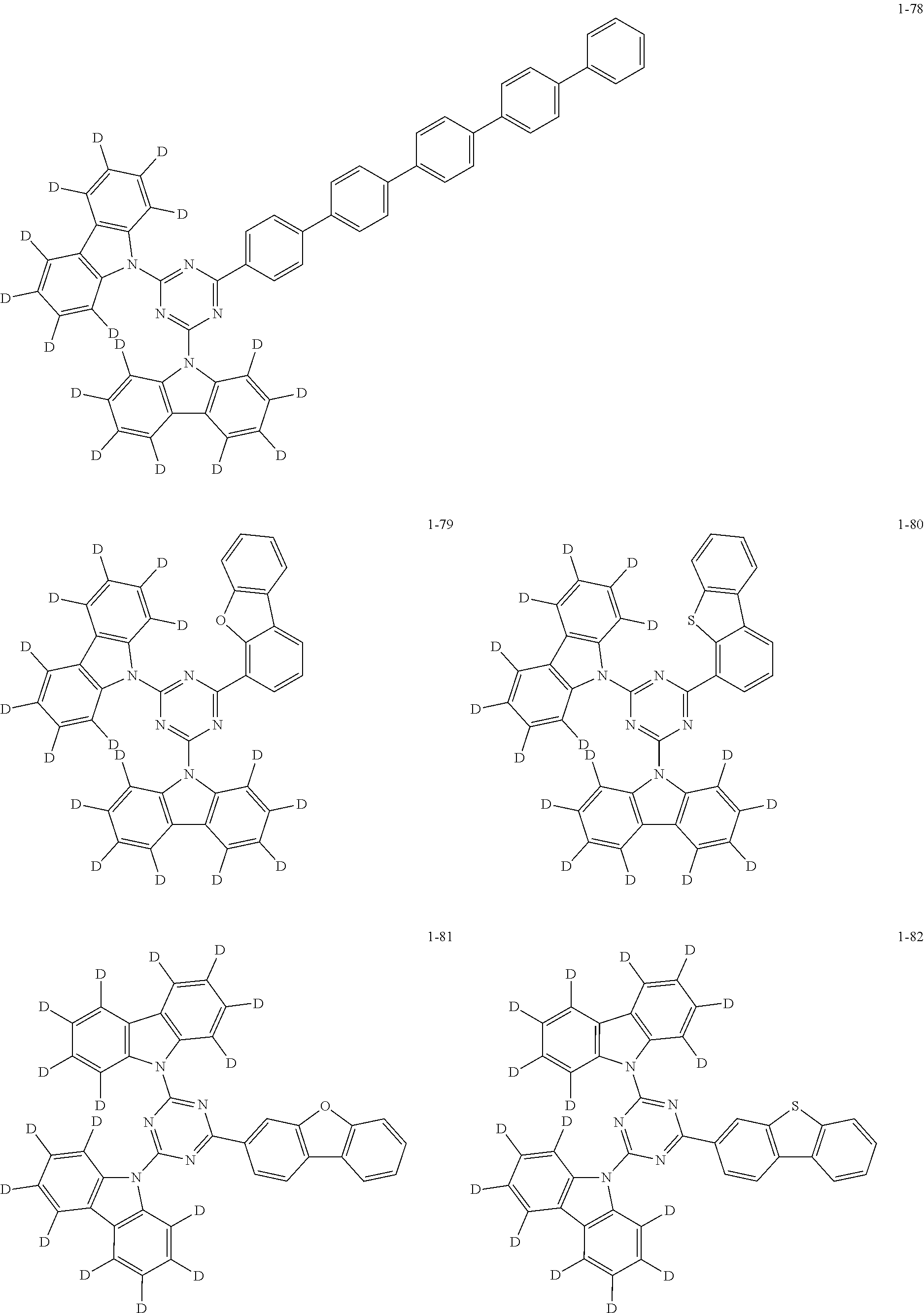

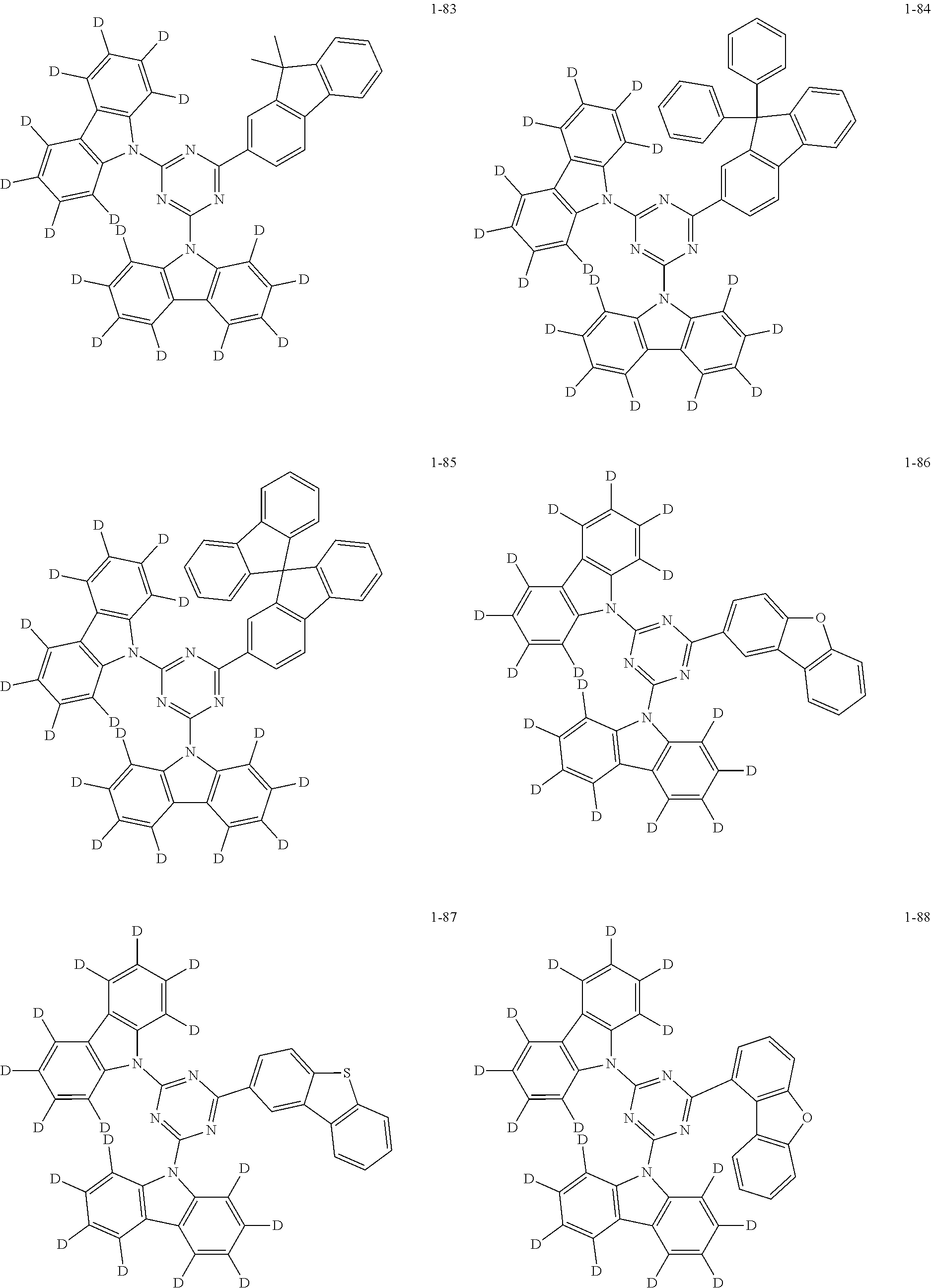

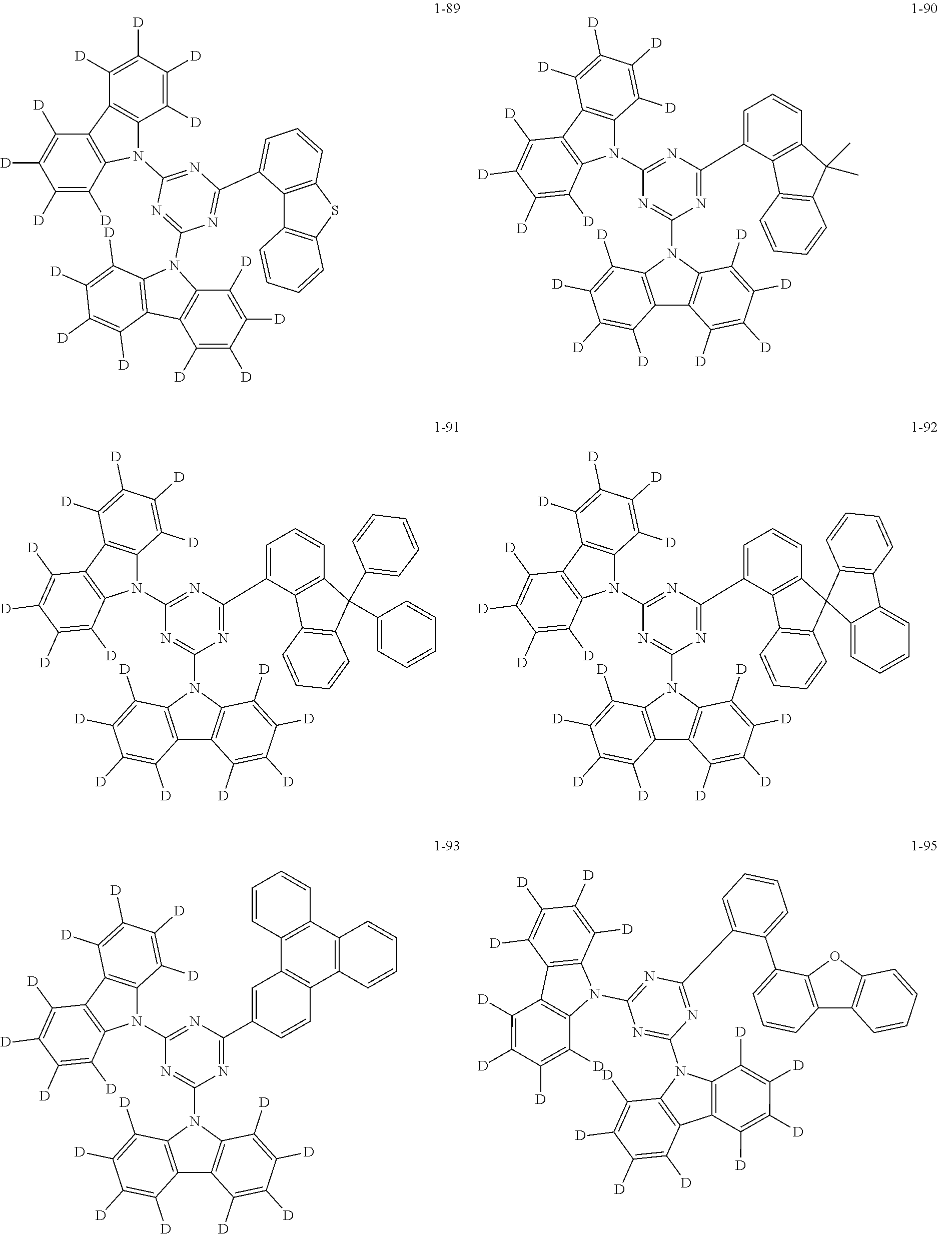

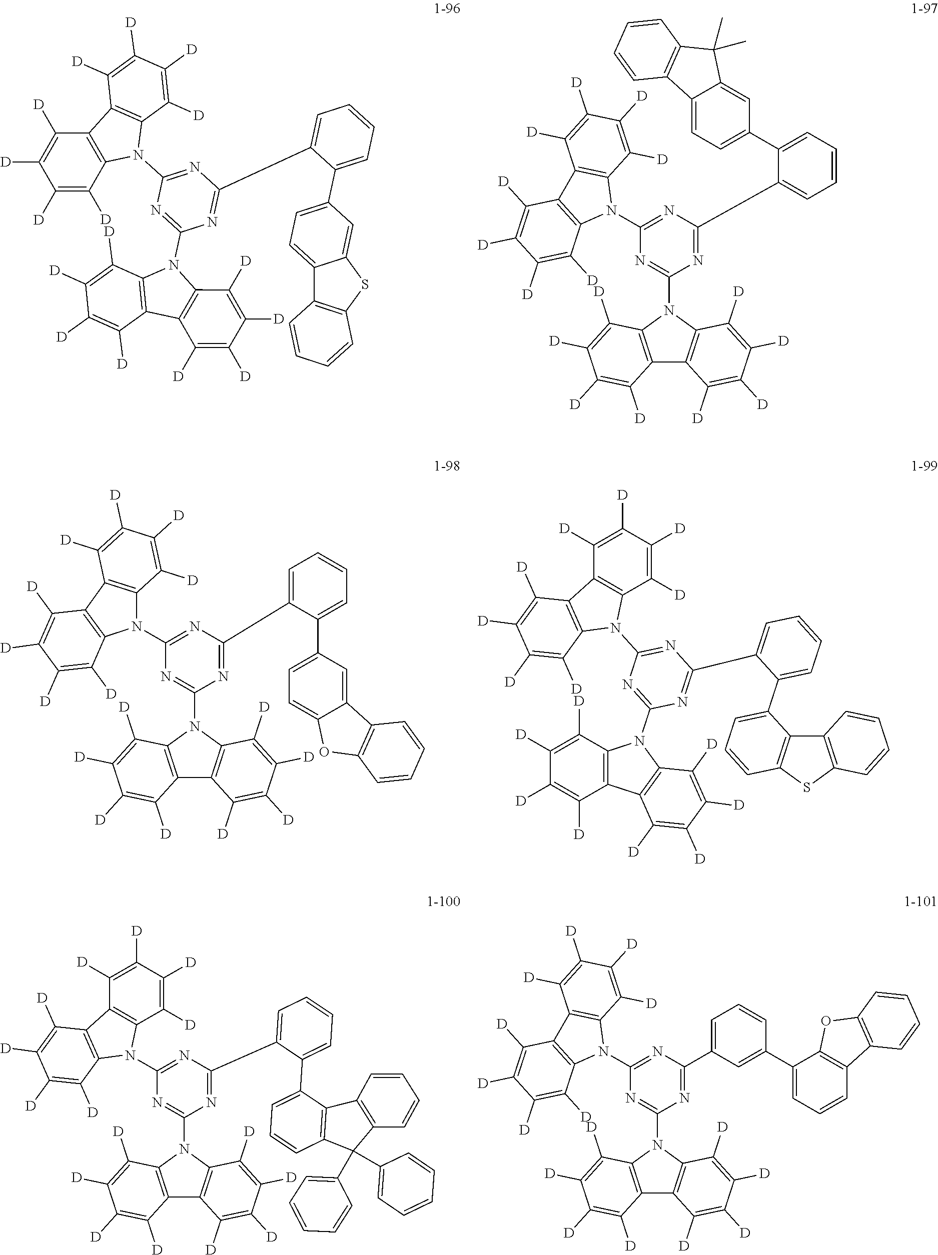

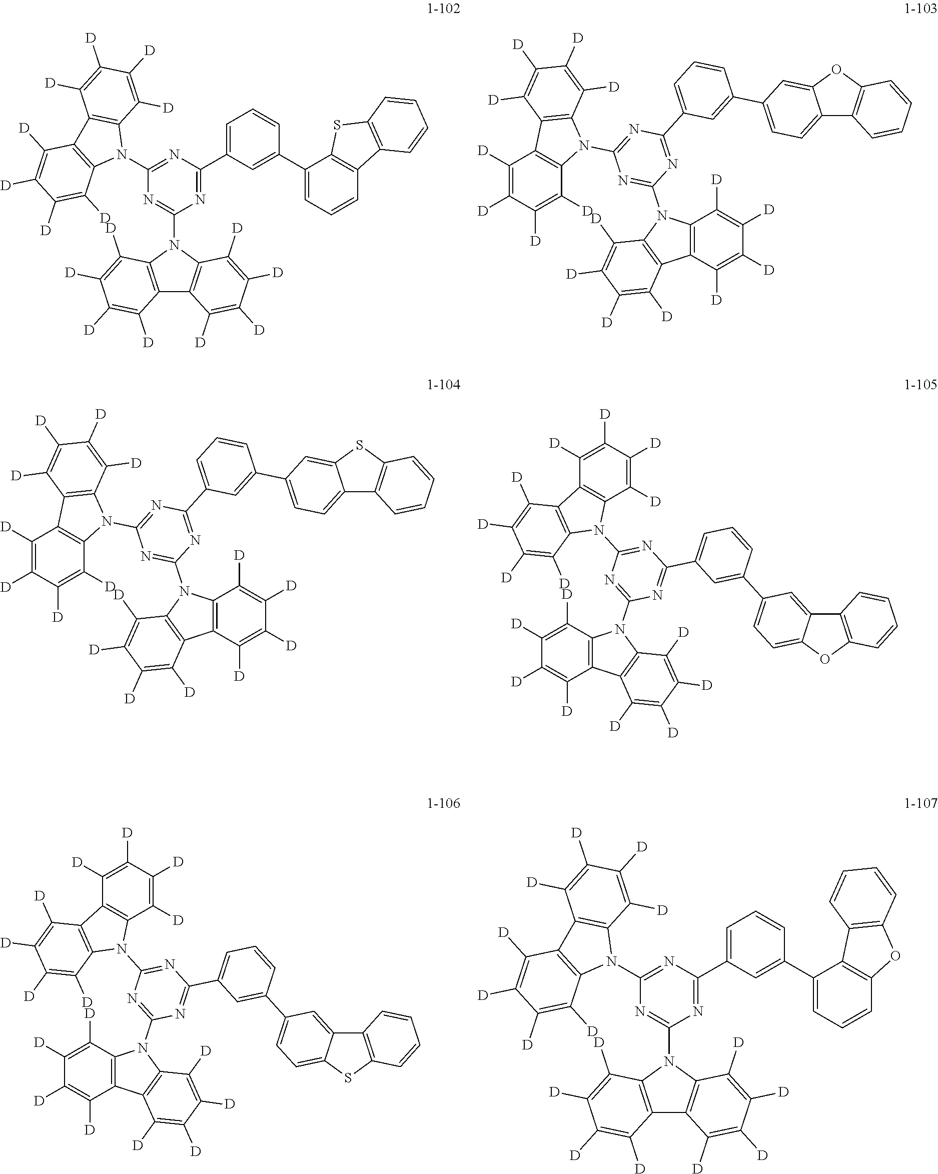

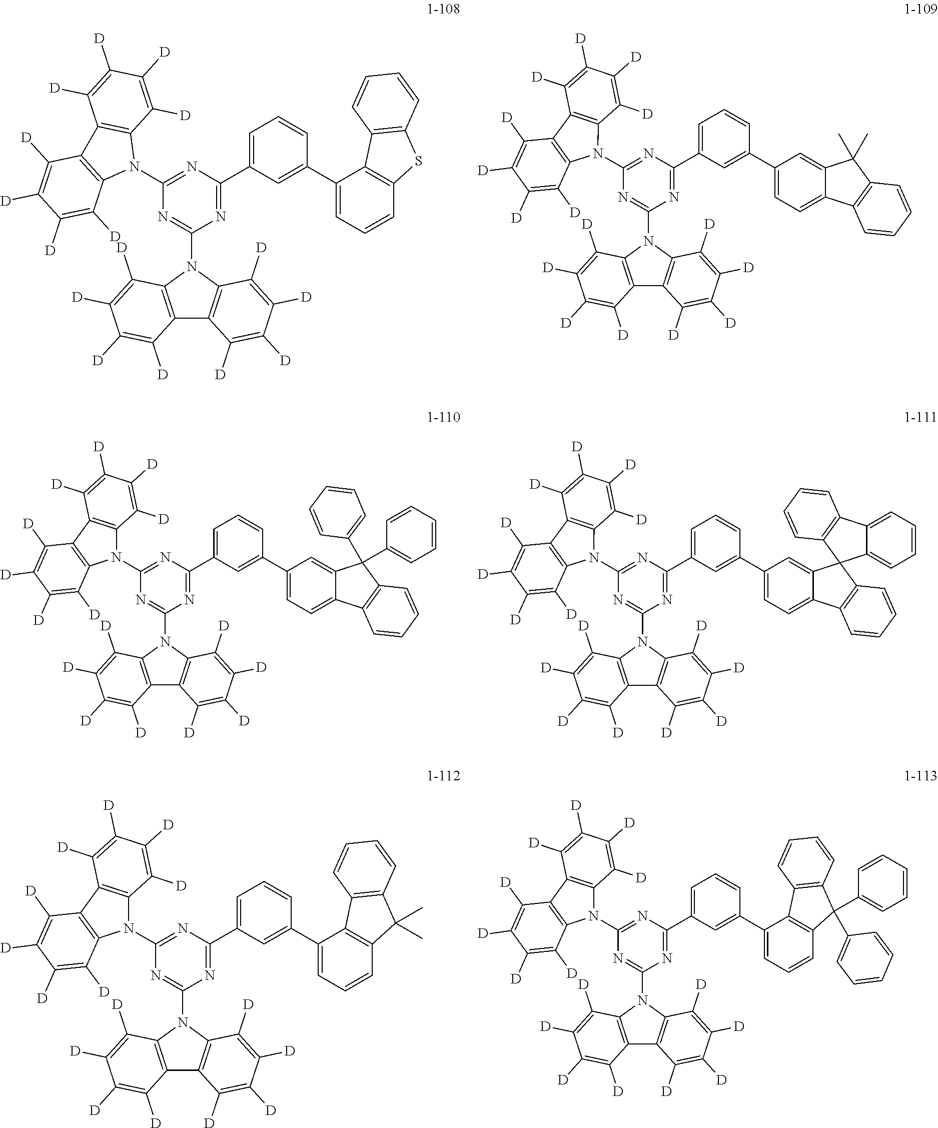

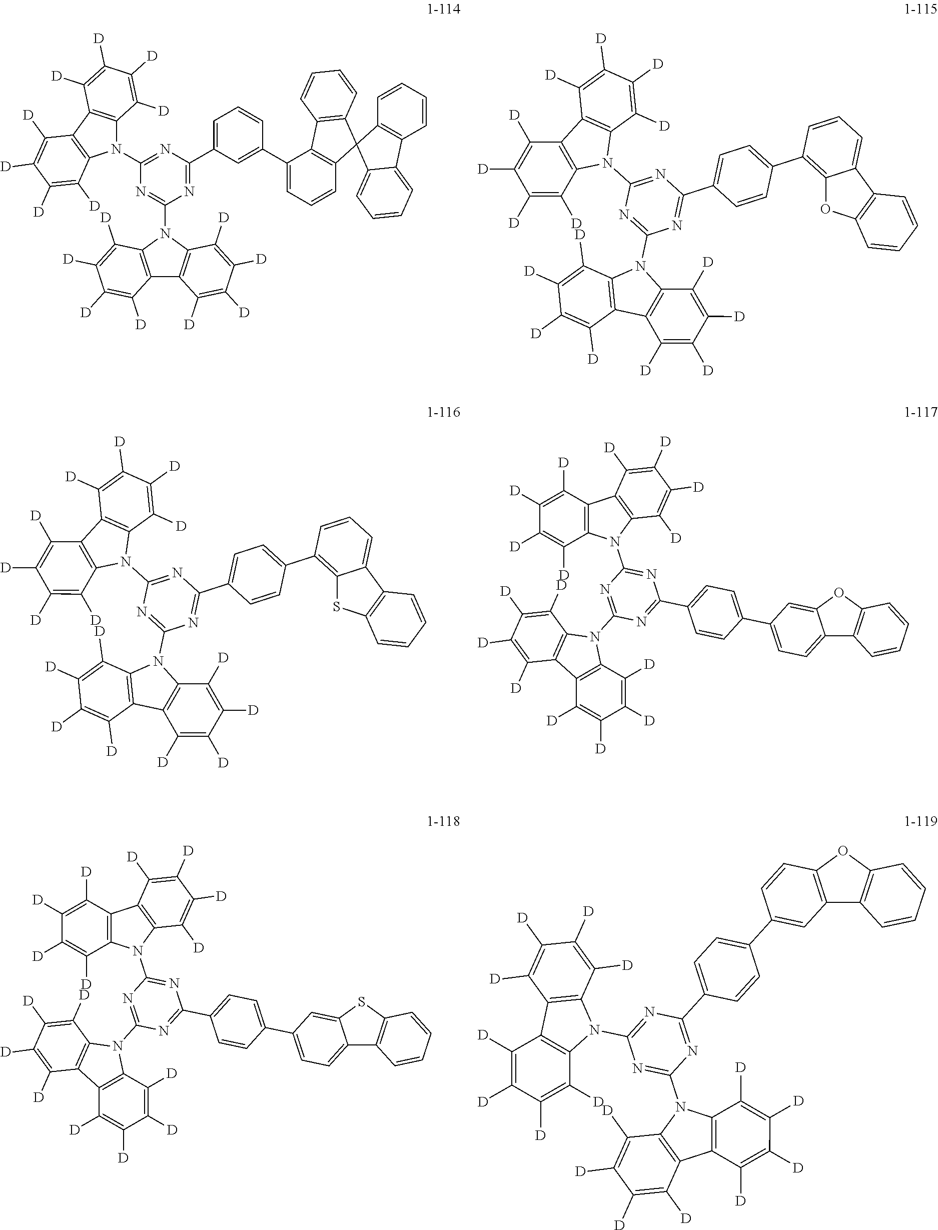

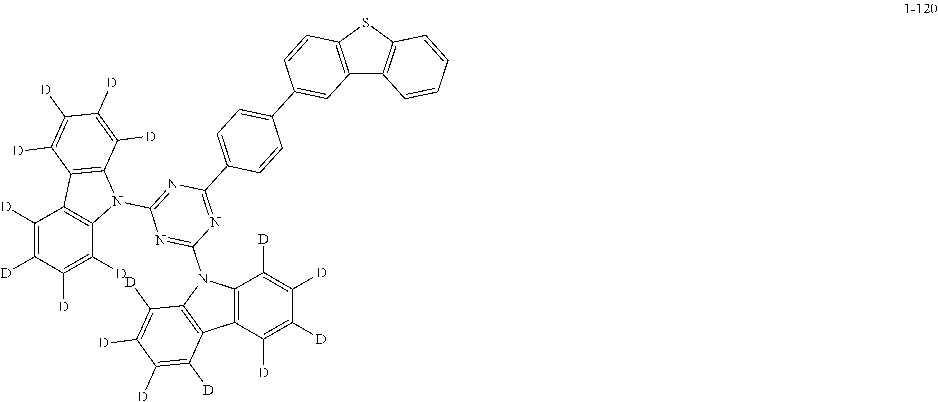 [SMILES: [2H]c1c([2H])c([2H])c2c(c1[2H])c1c([2H])c([2H])c([2H])c([2H])c1n2-c1nc(-c2ccc(-c3ccc4sc5ccccc5c4c3)cc2)nc(-n2c3c([2H])c([2H])c([2H])c([2H])c3c3c([2H])c([2H])c([2H])c([2H])c32)n1]

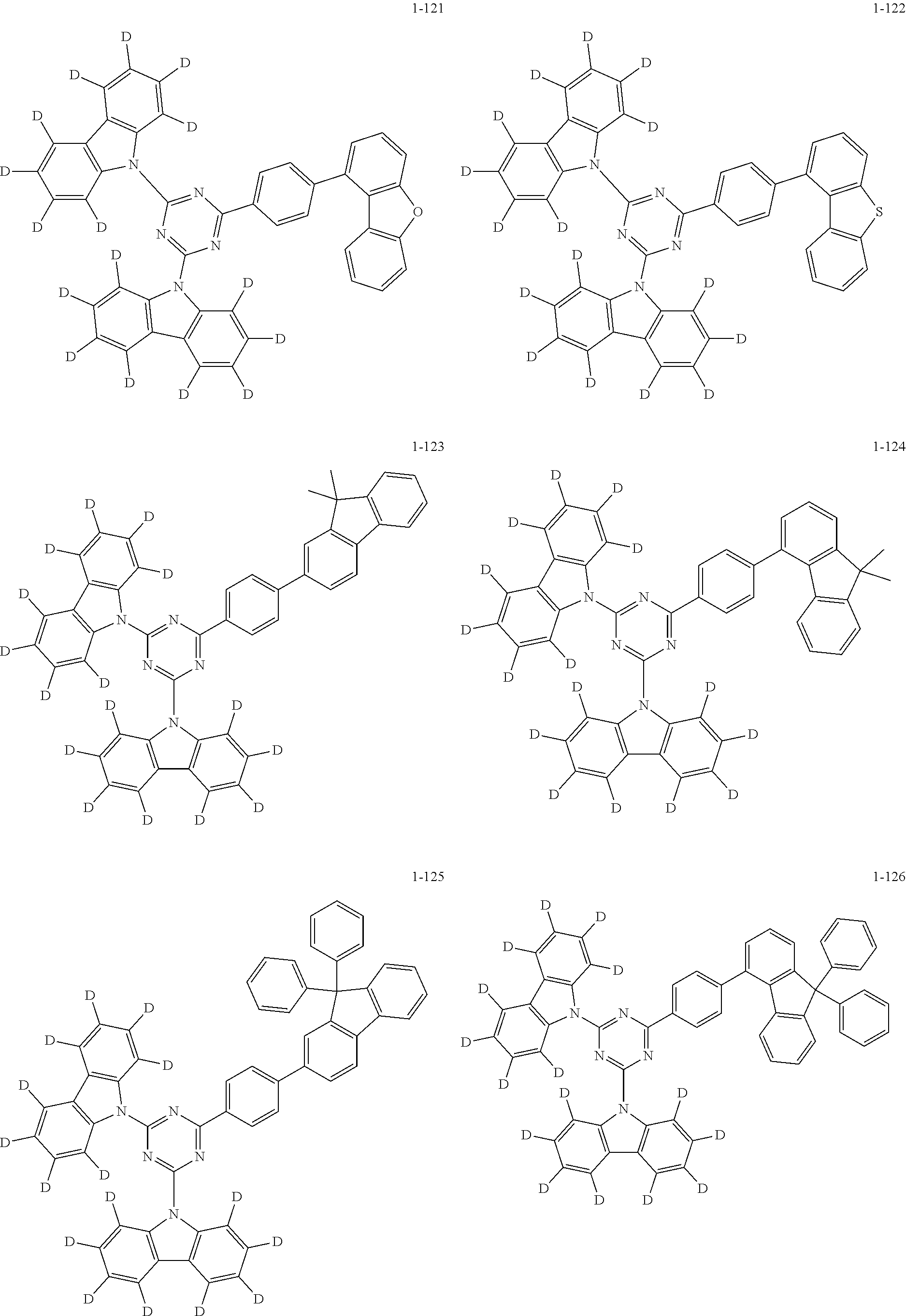

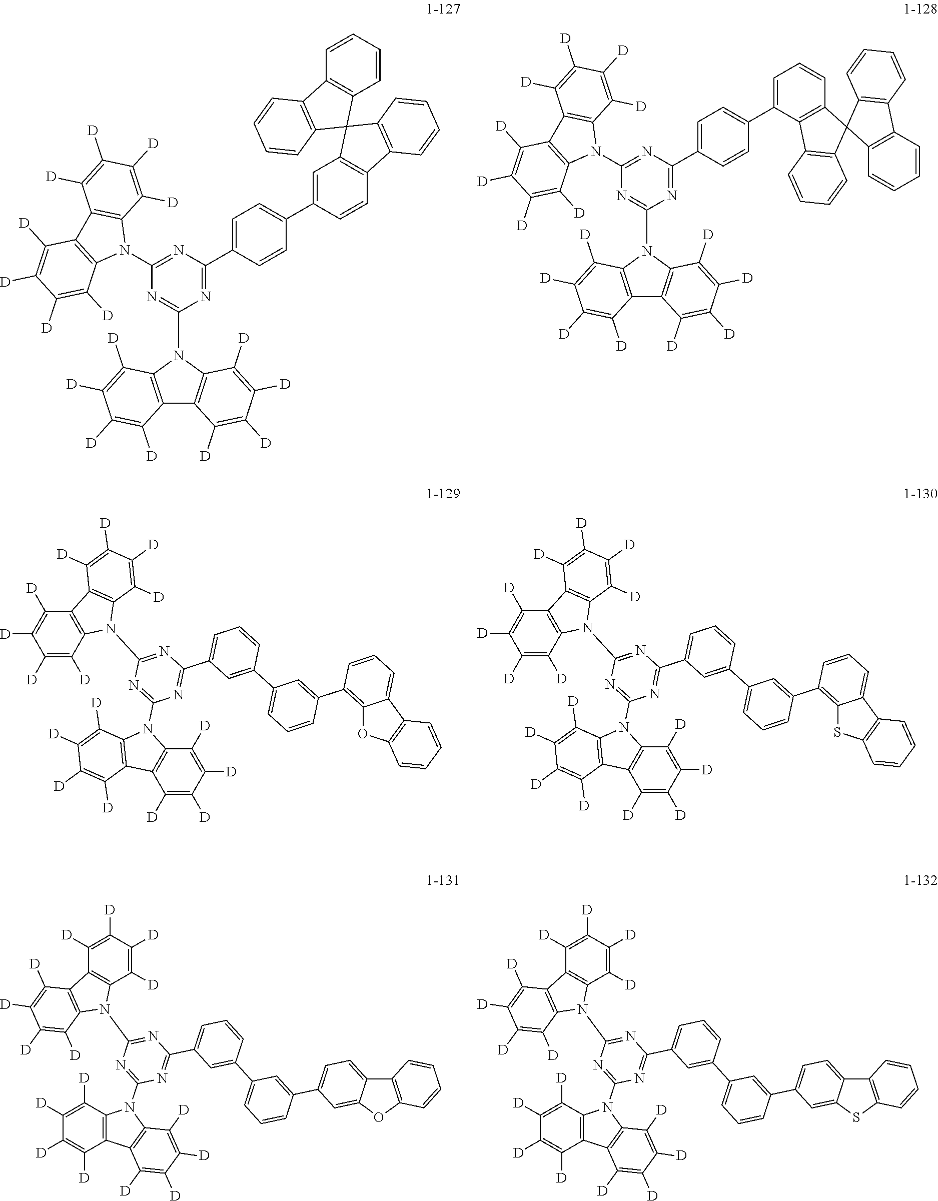

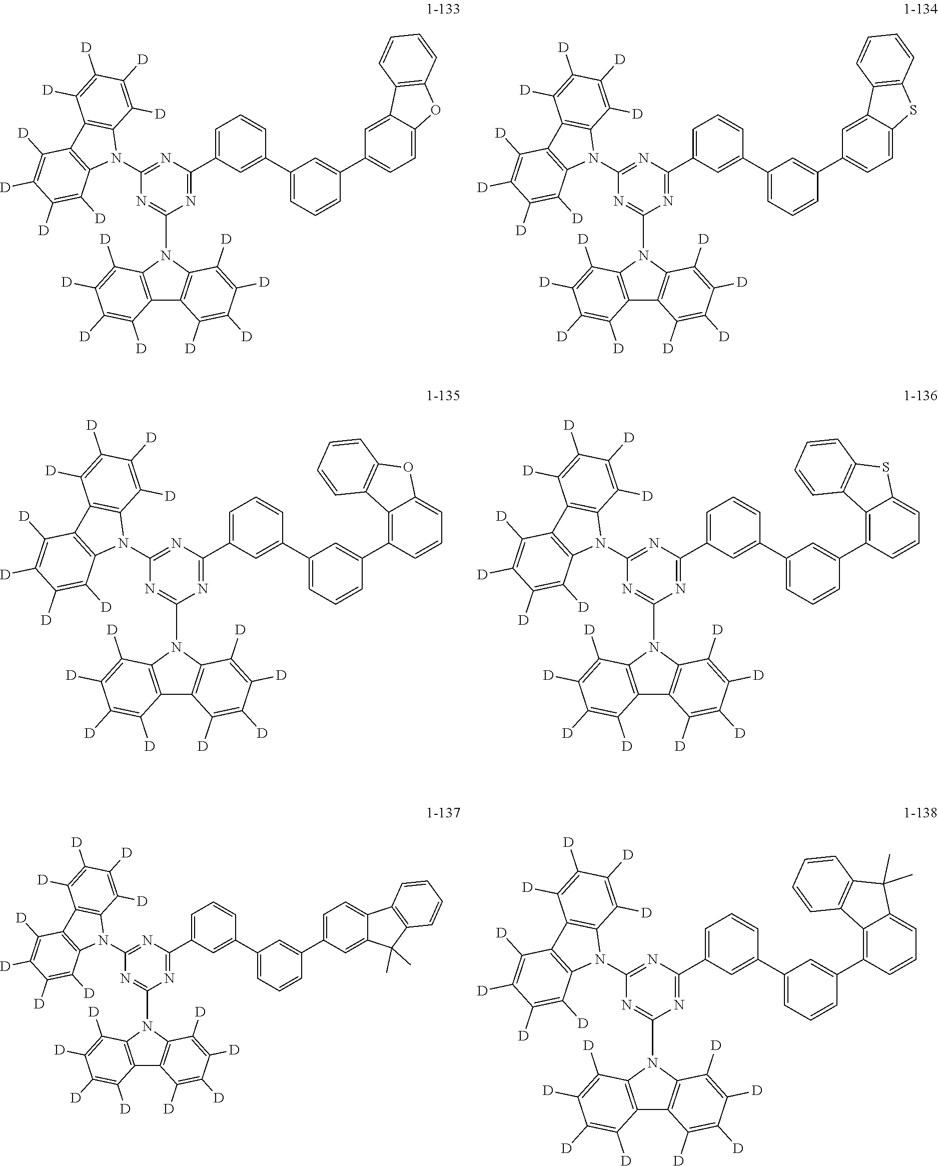

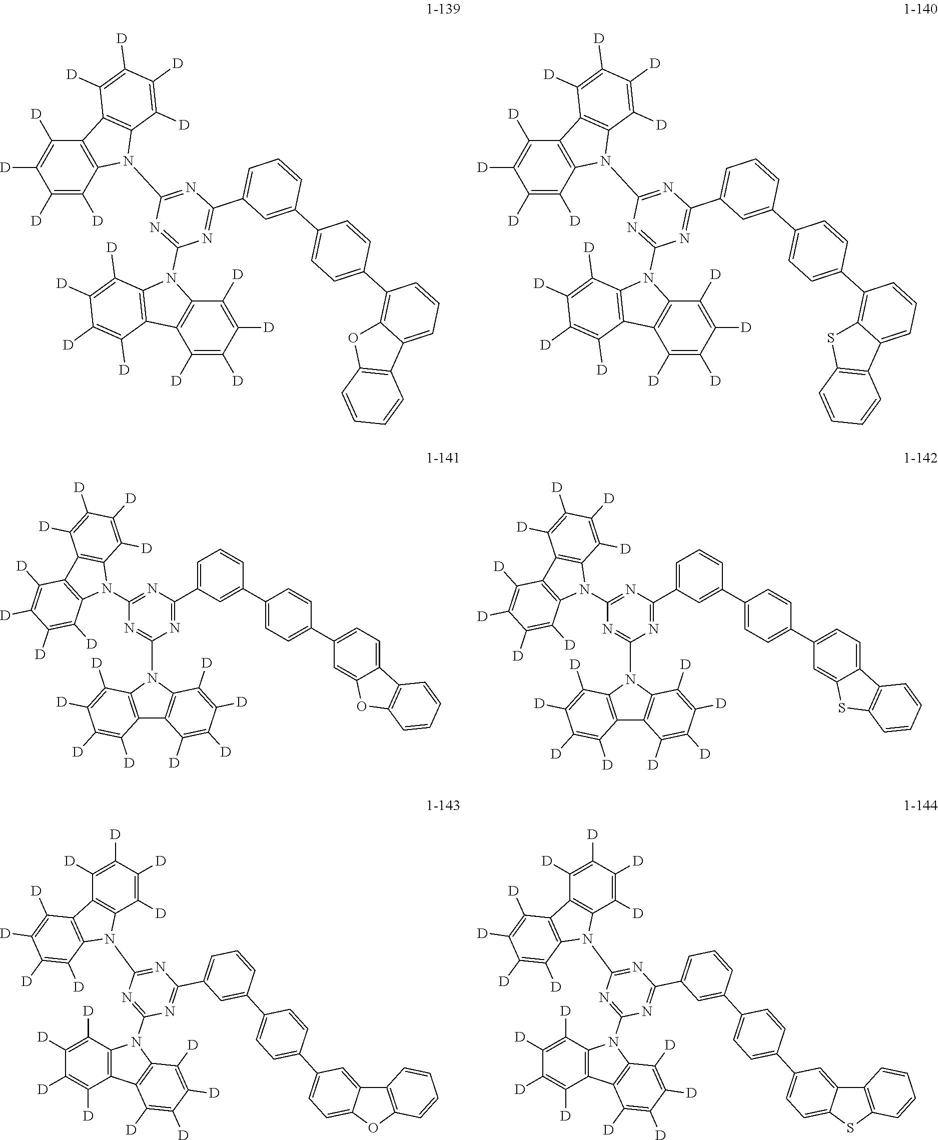

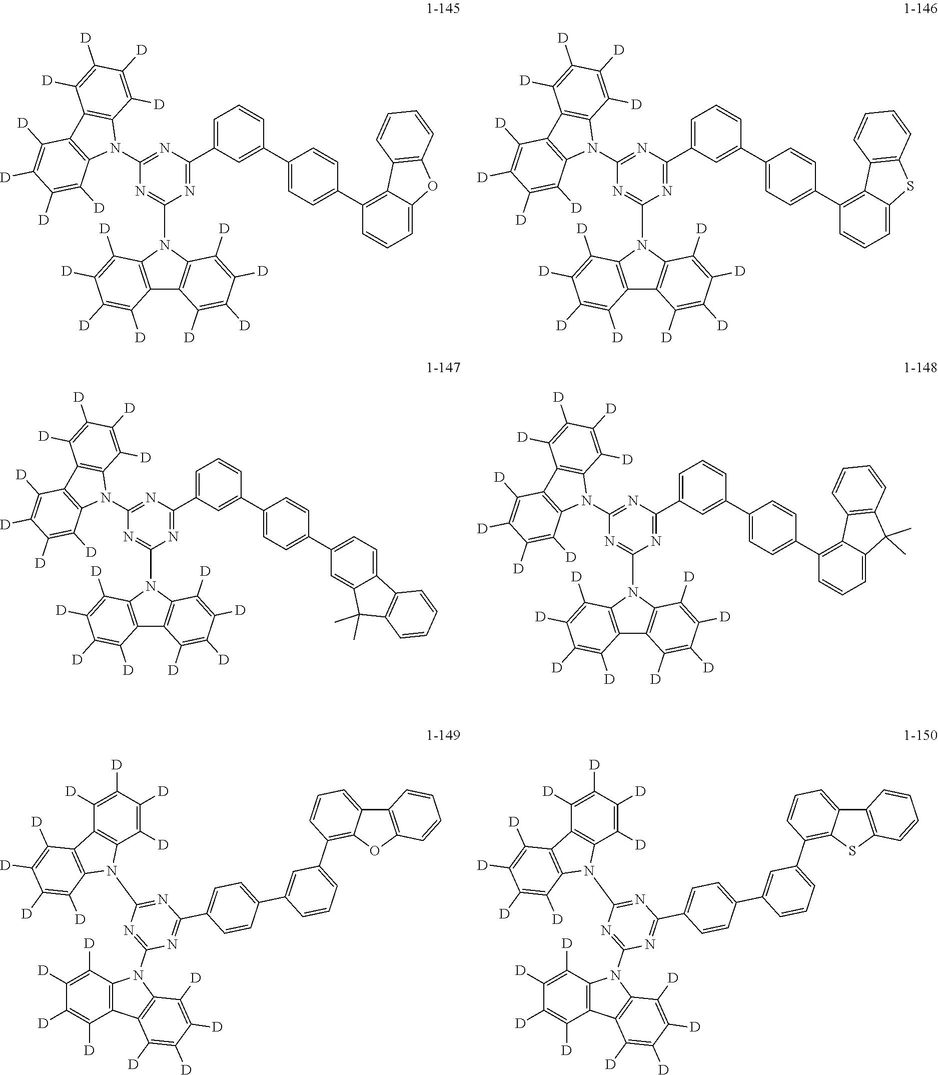

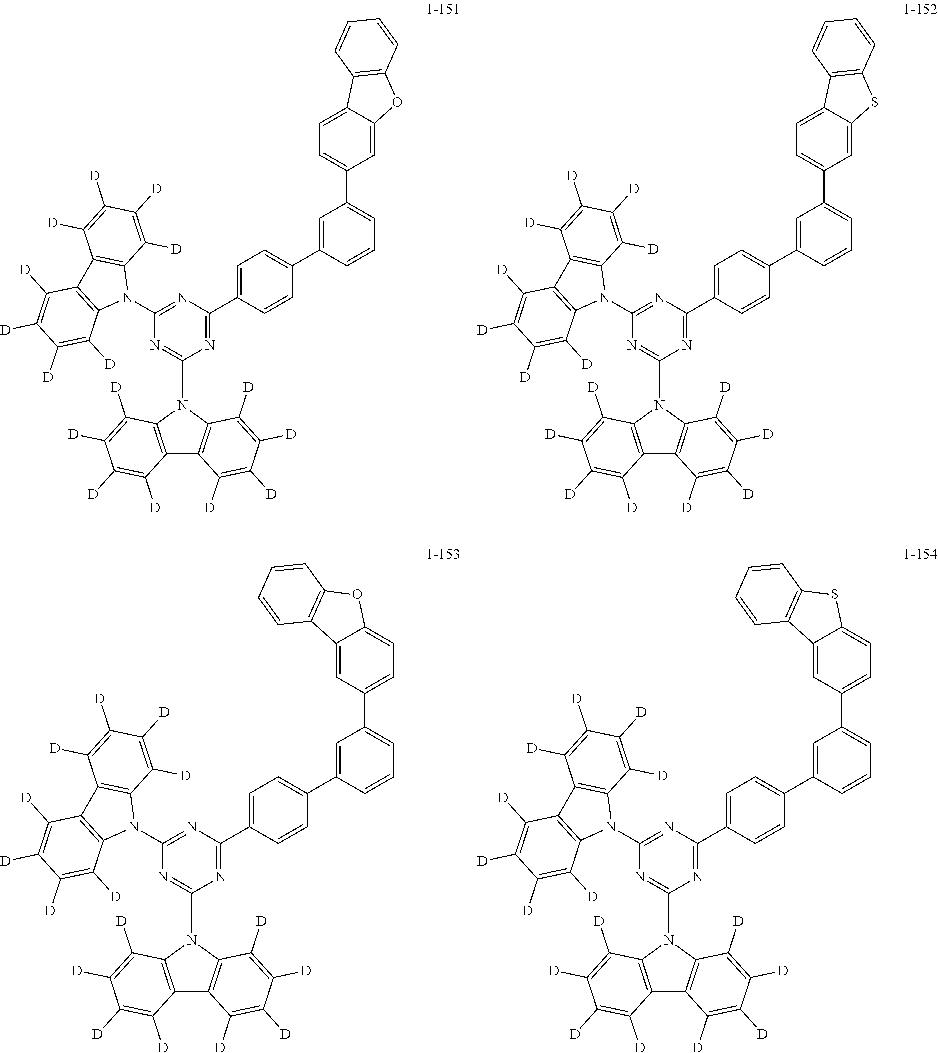

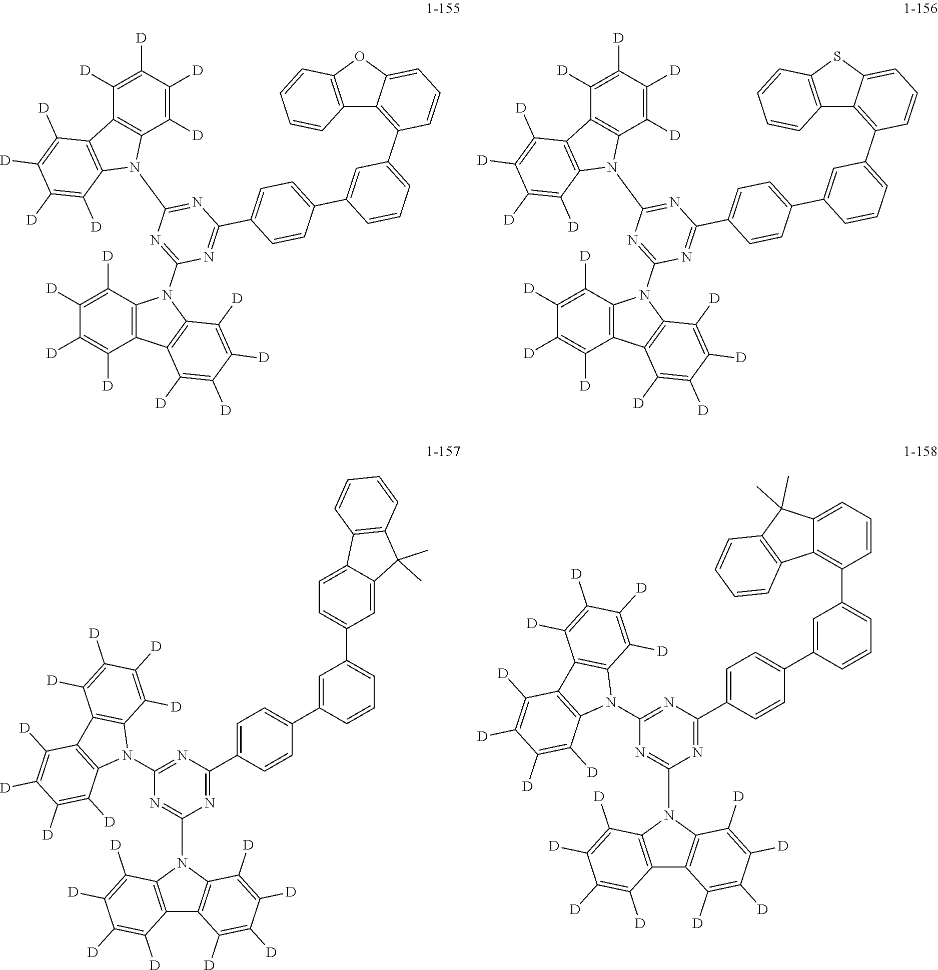

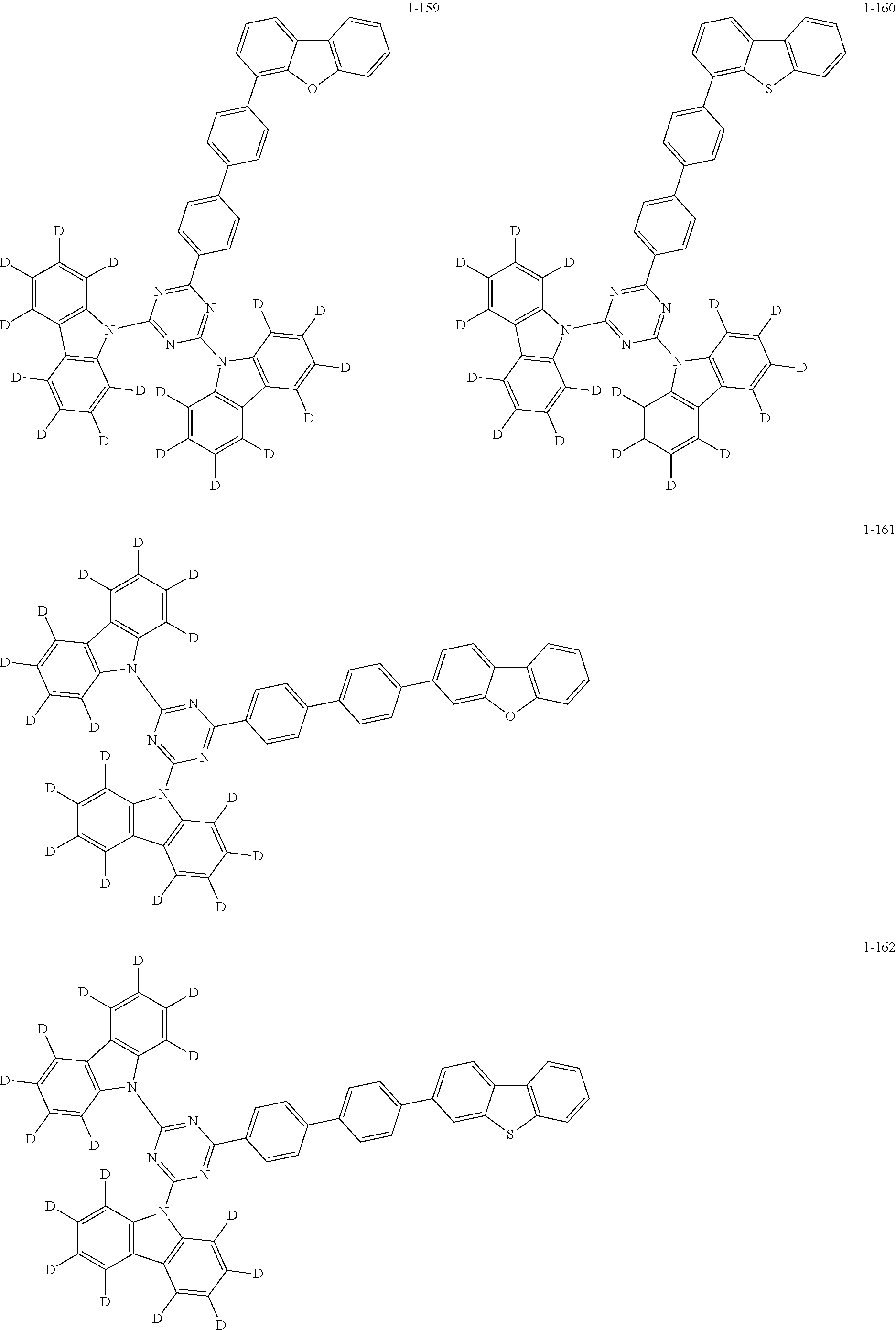

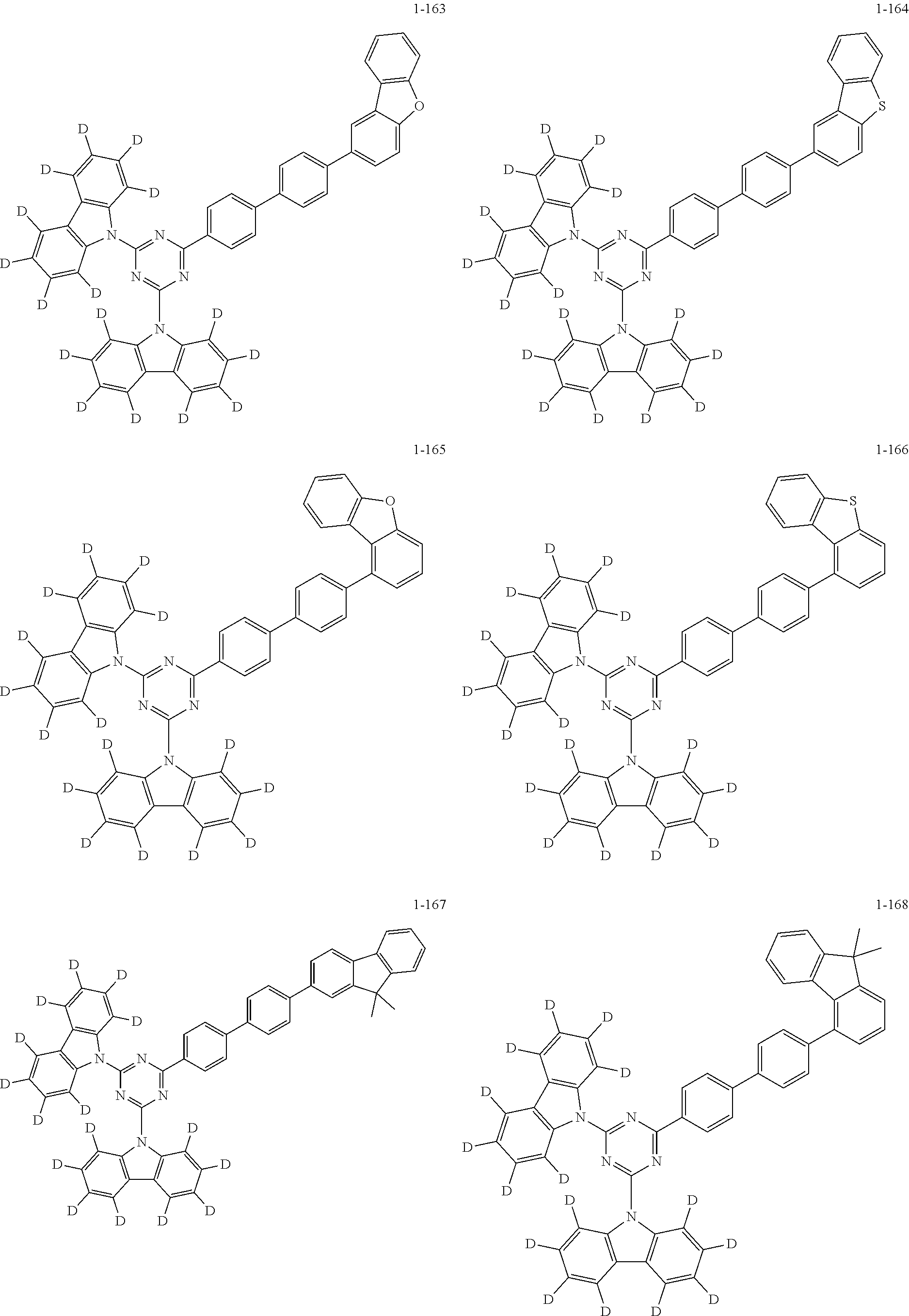

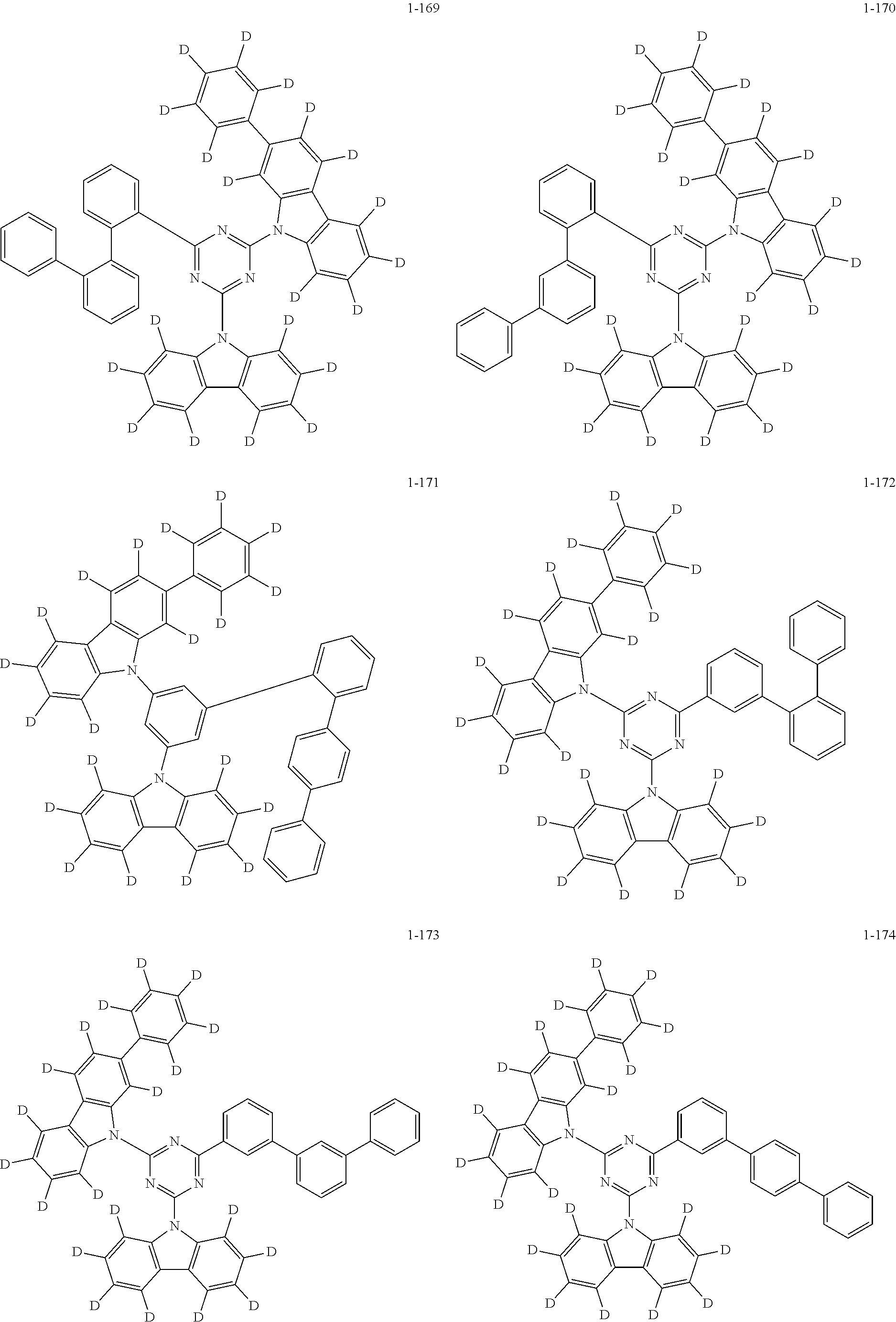

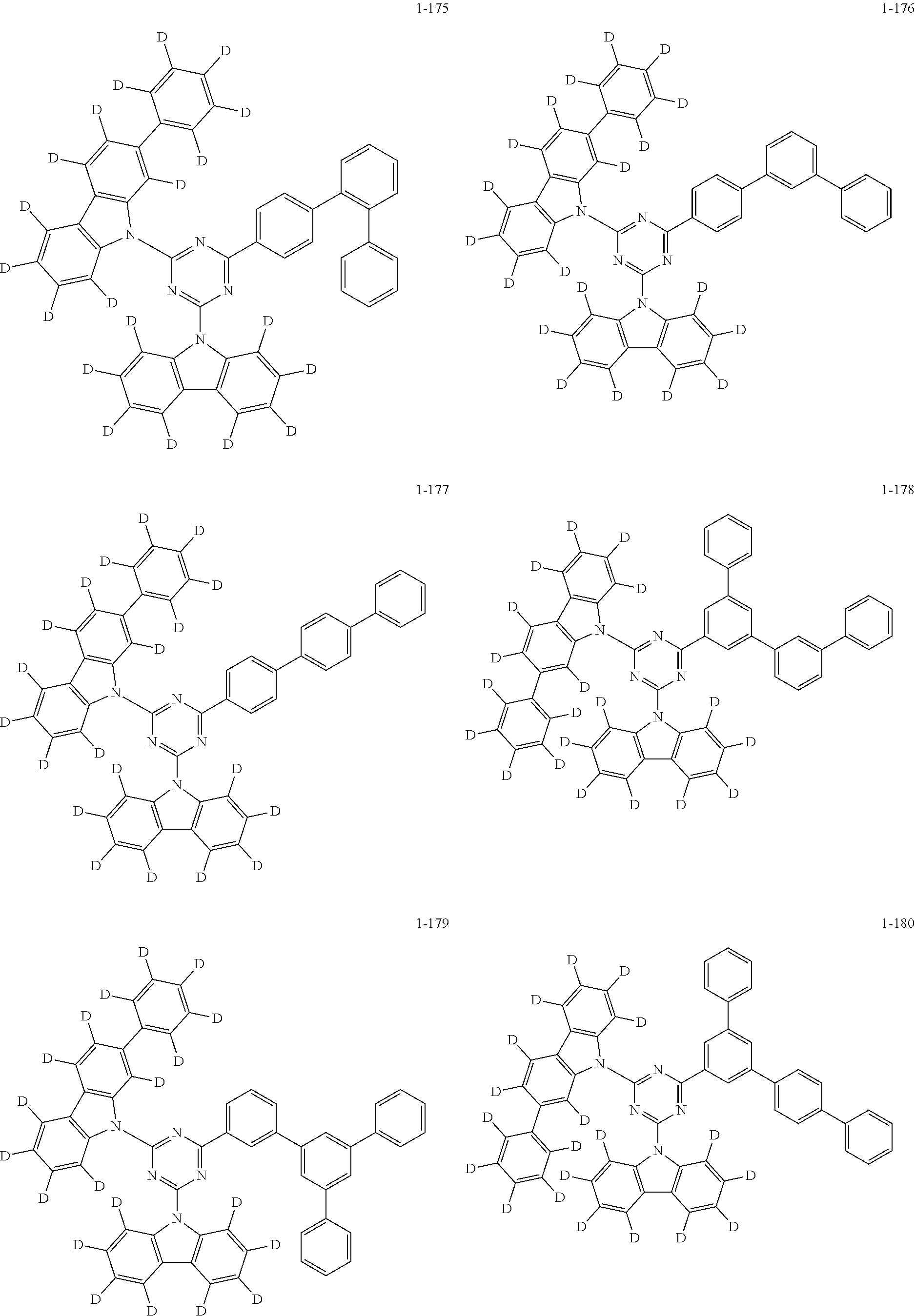

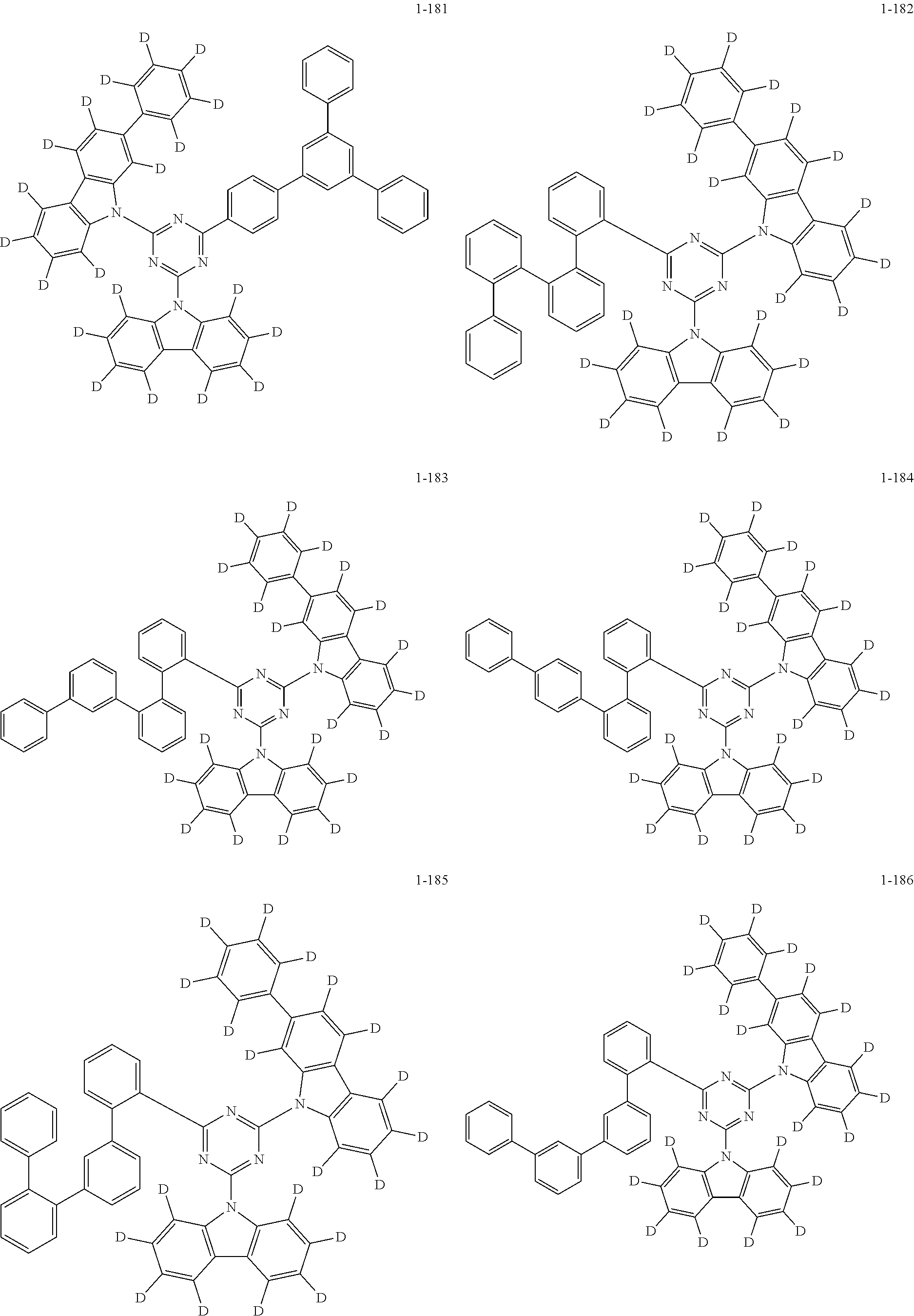

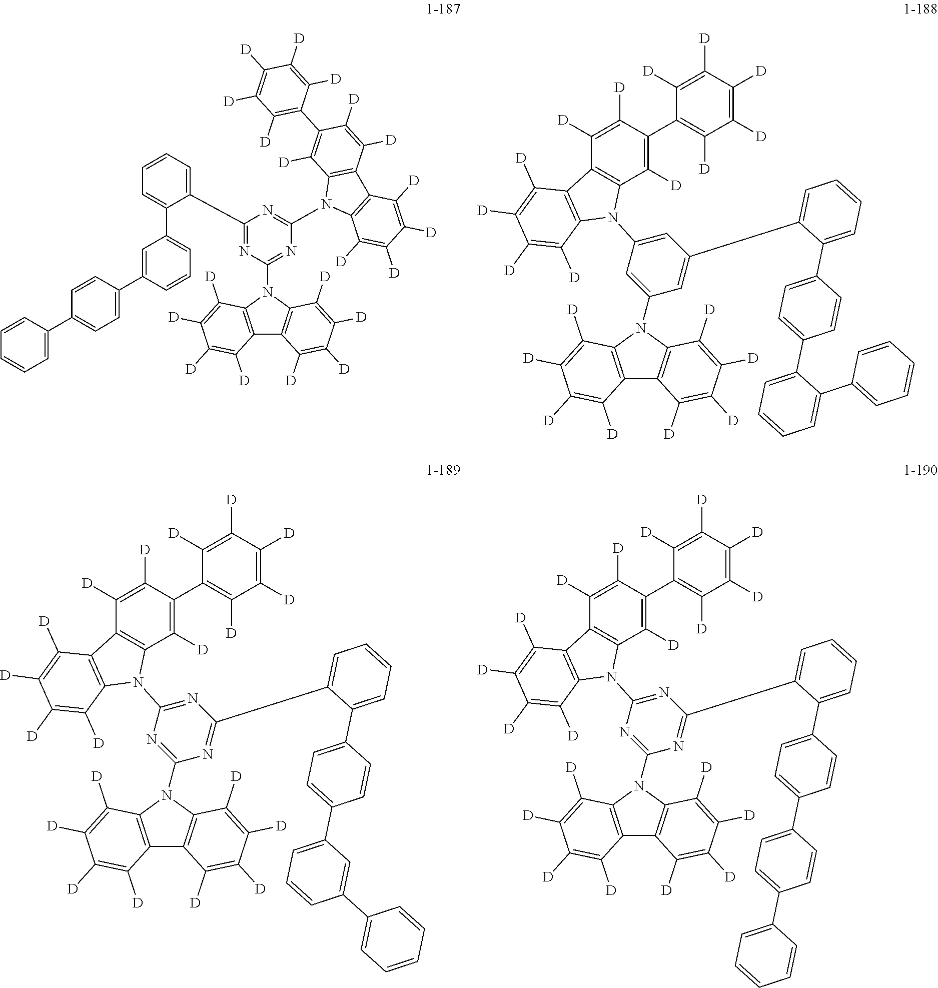

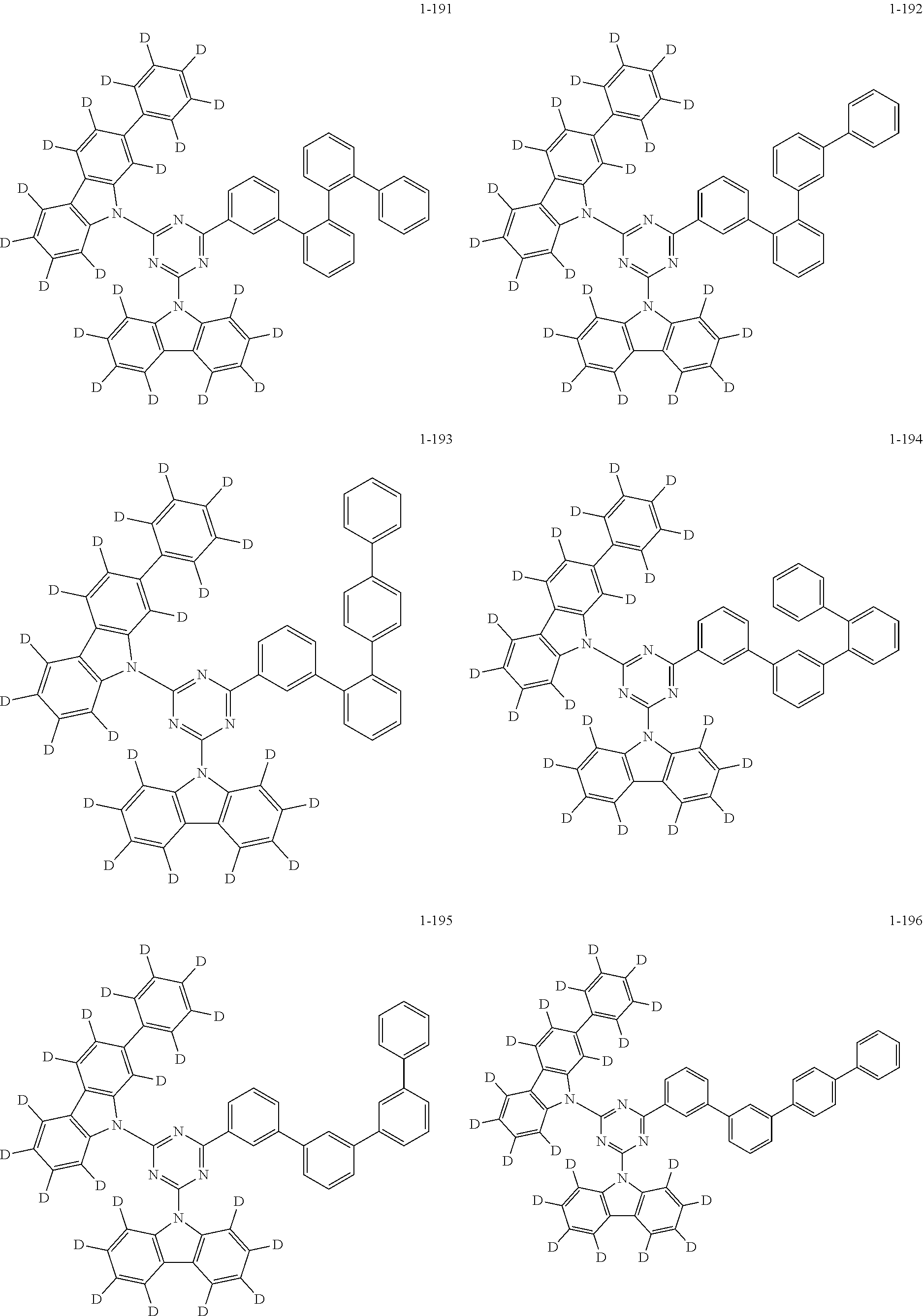

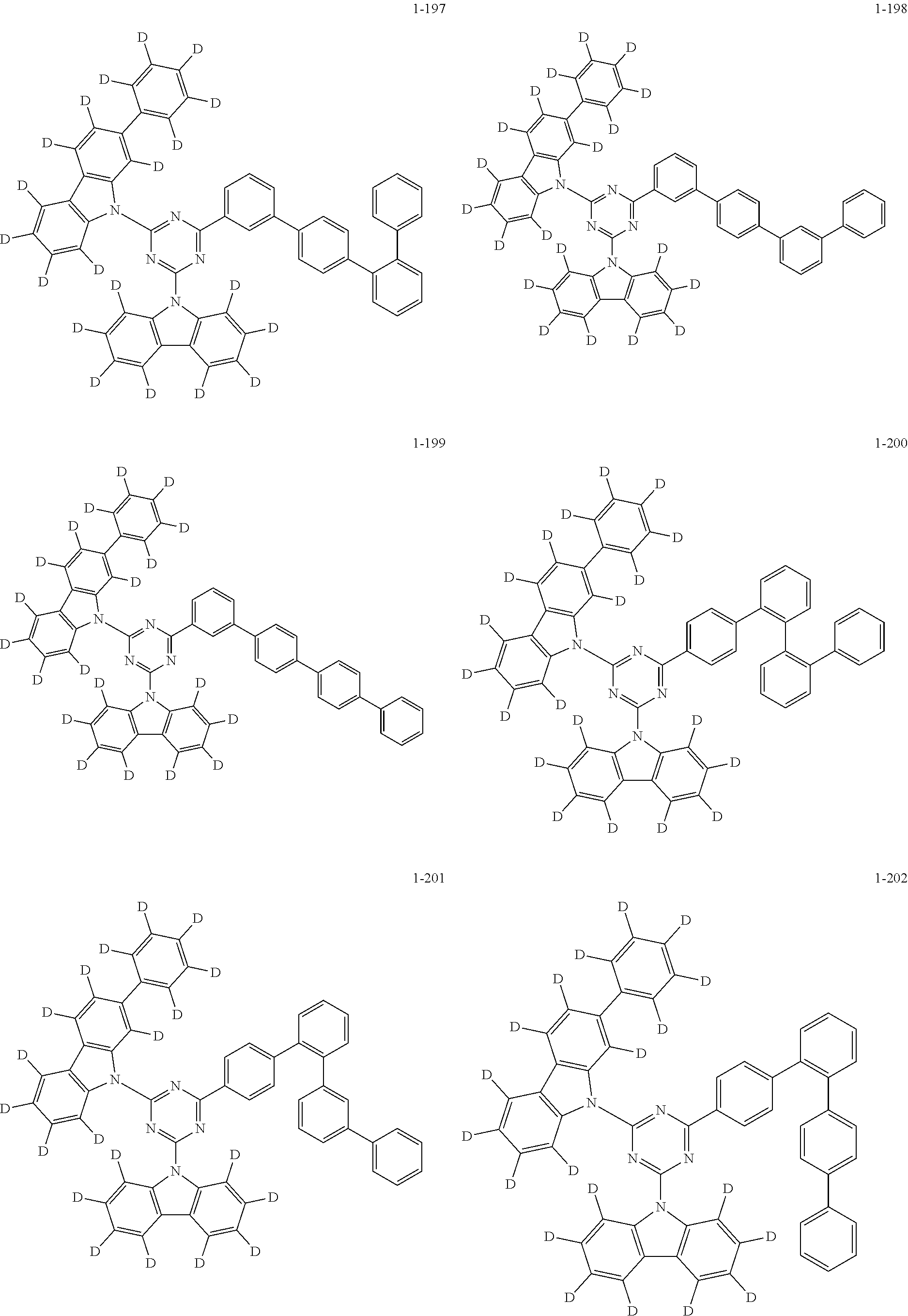

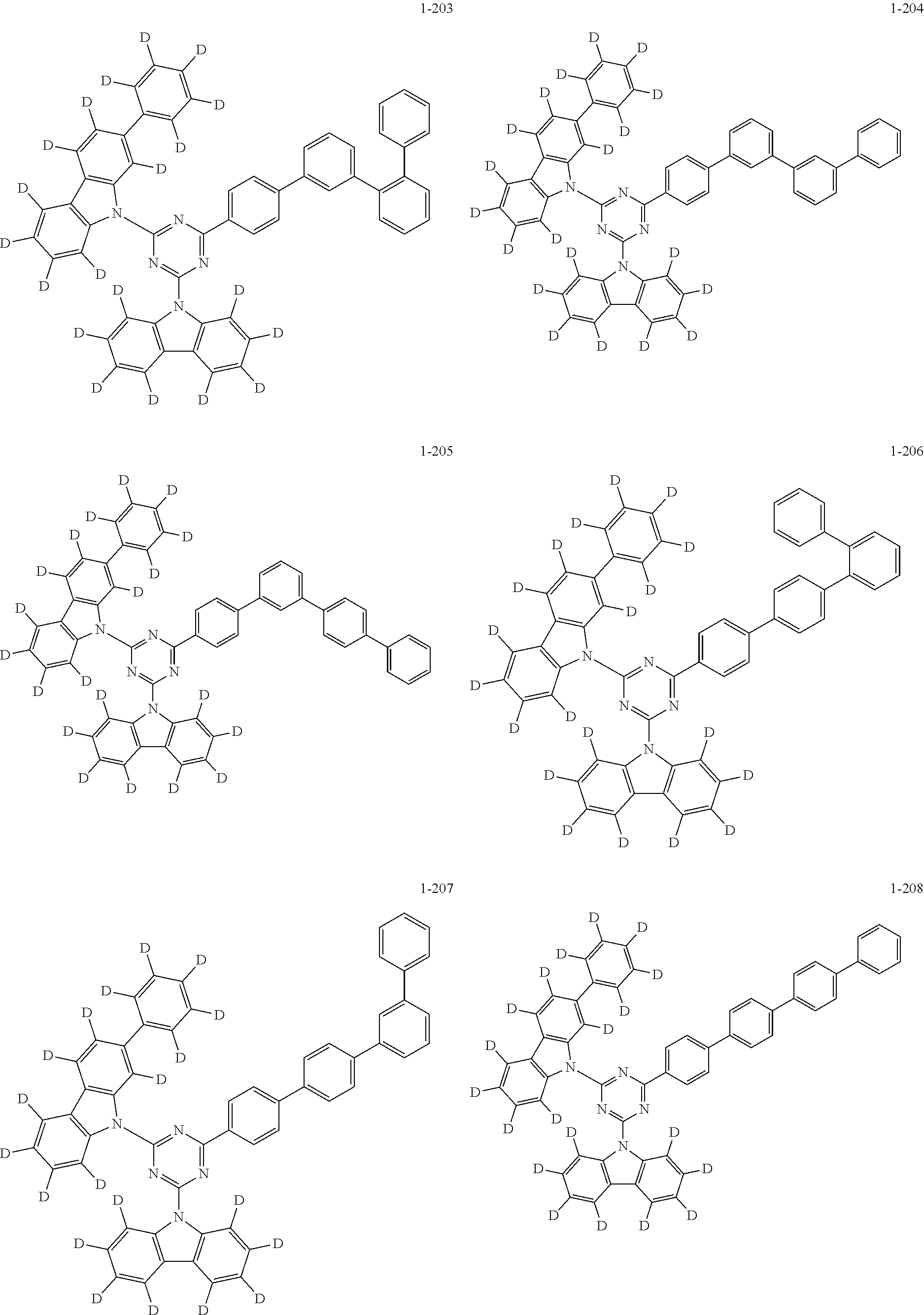

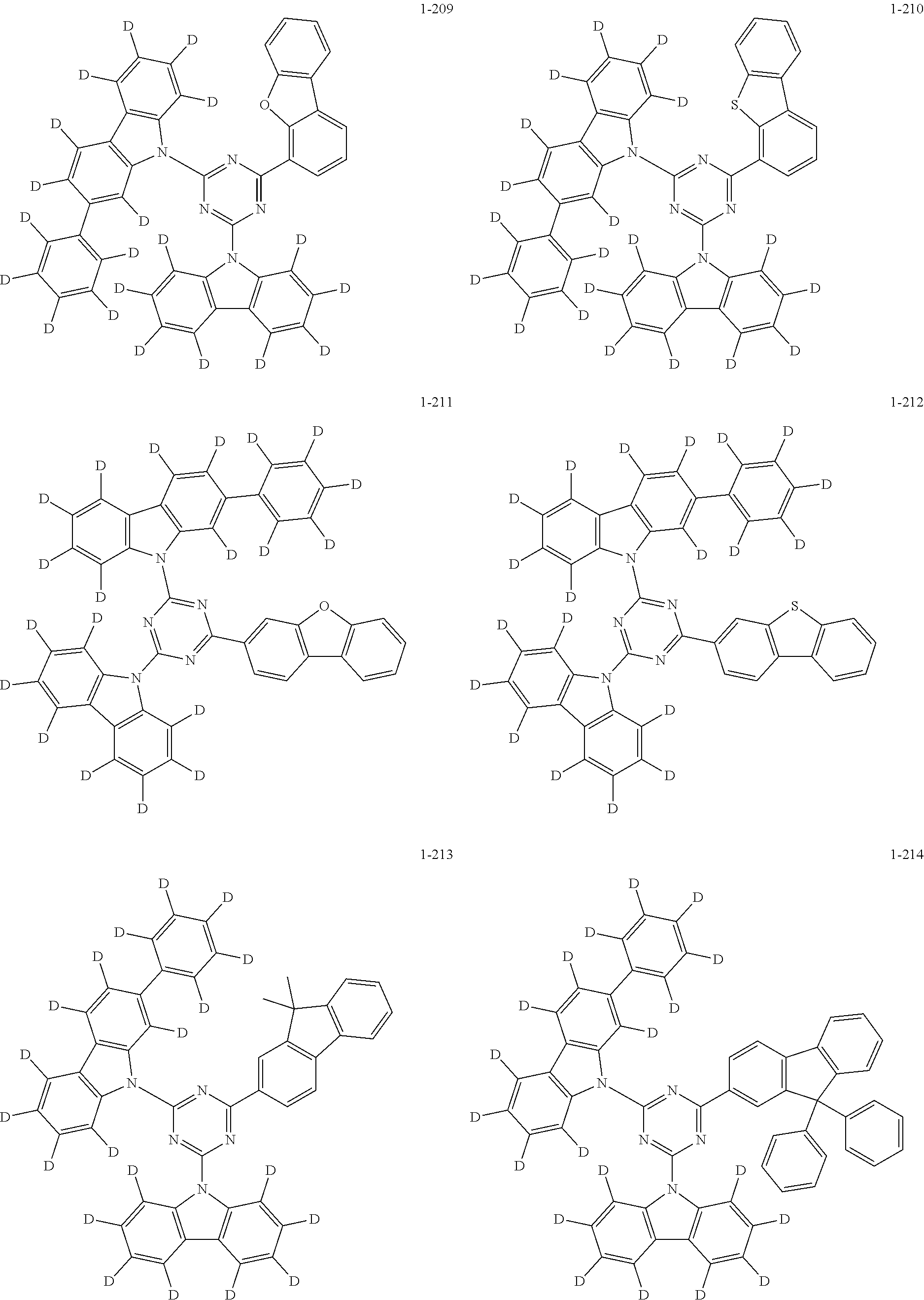

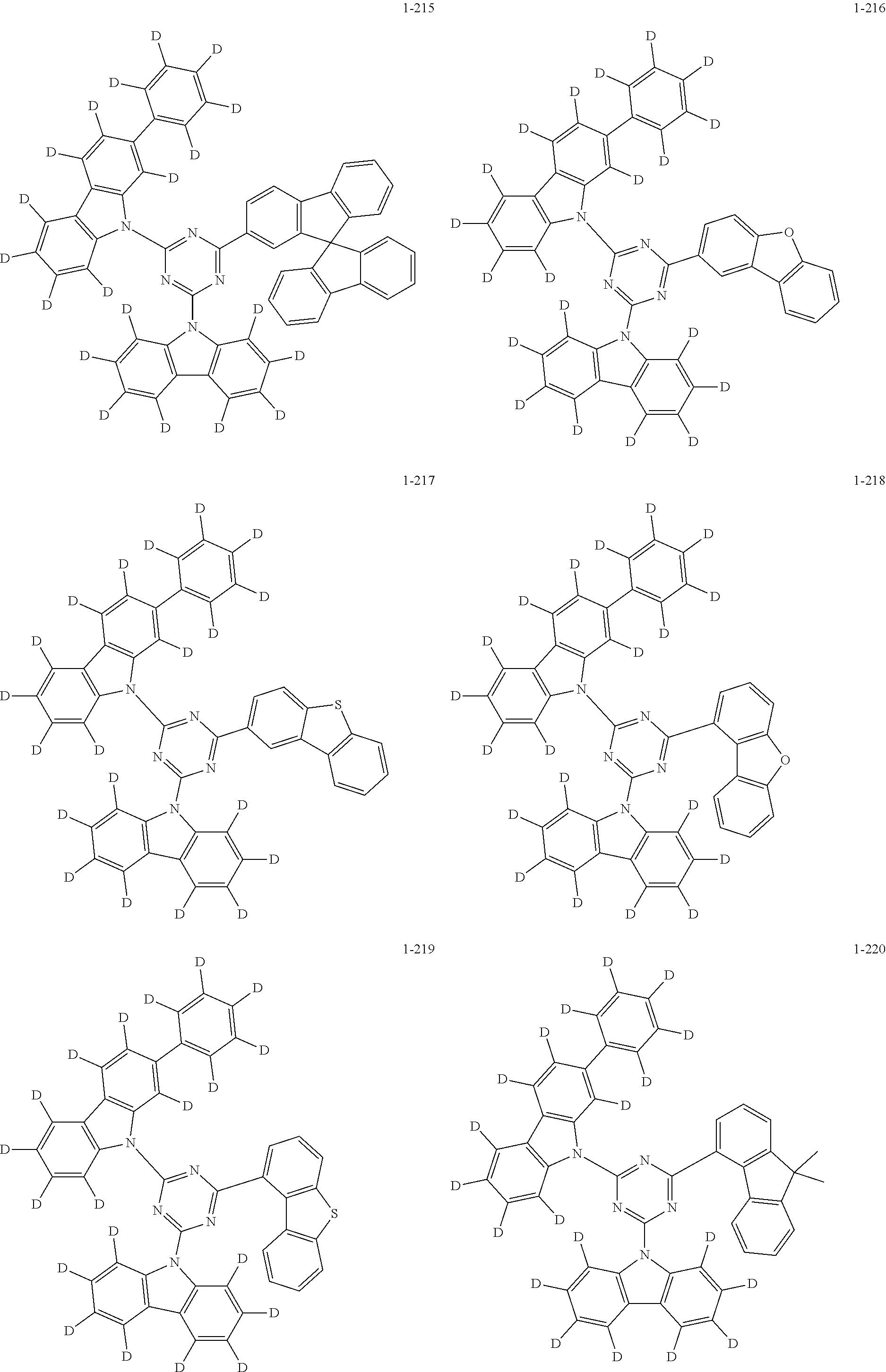

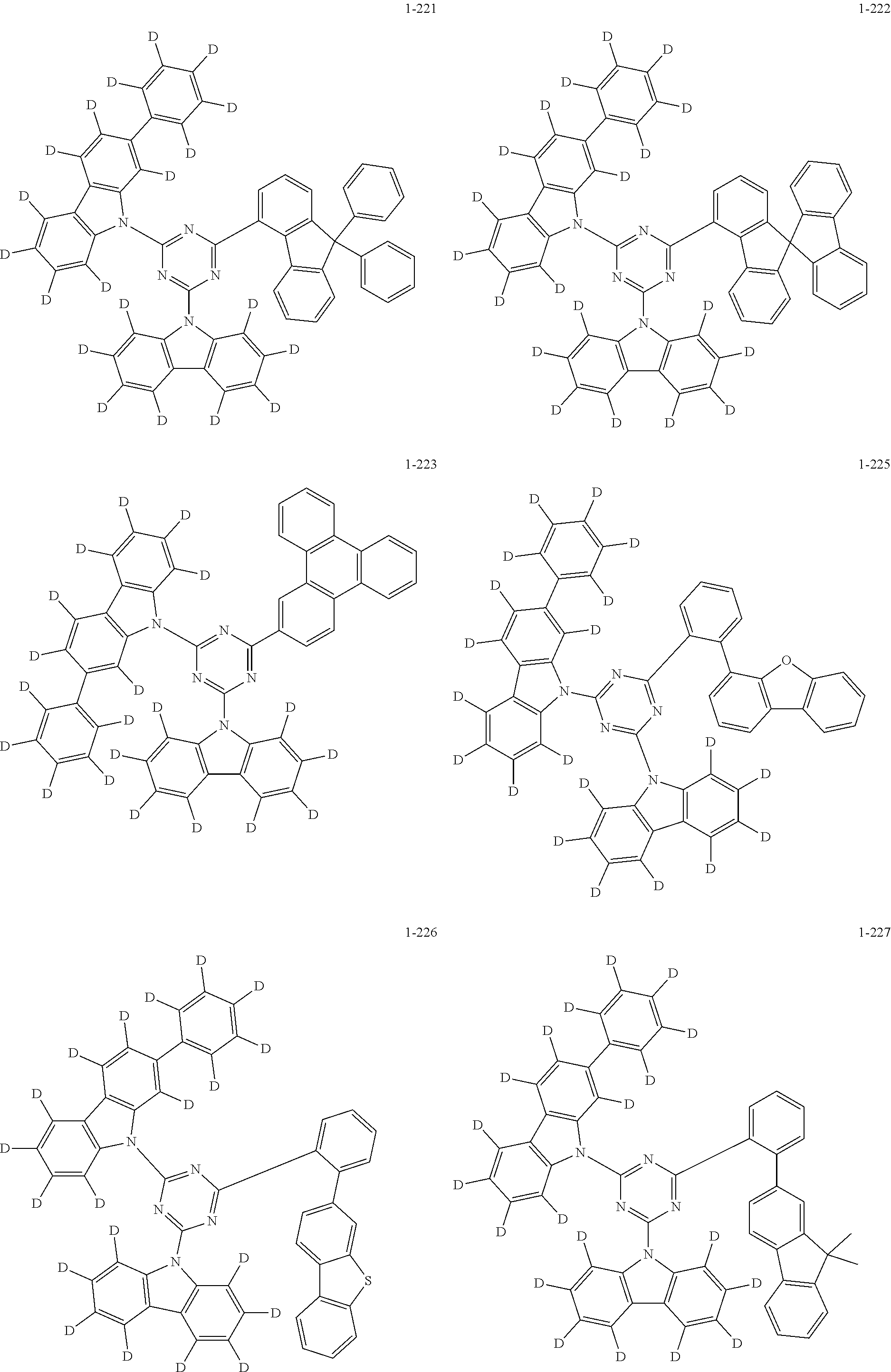

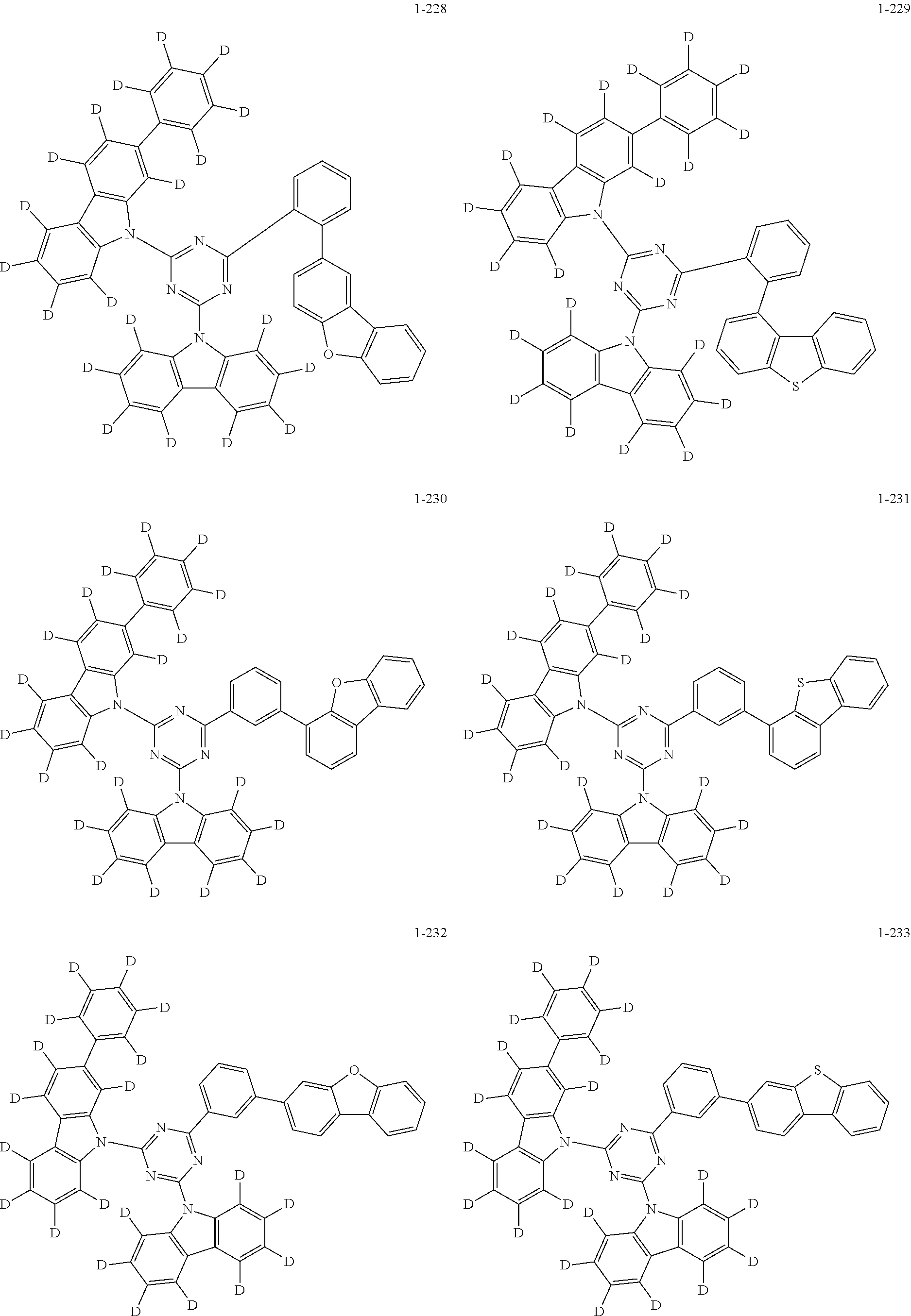

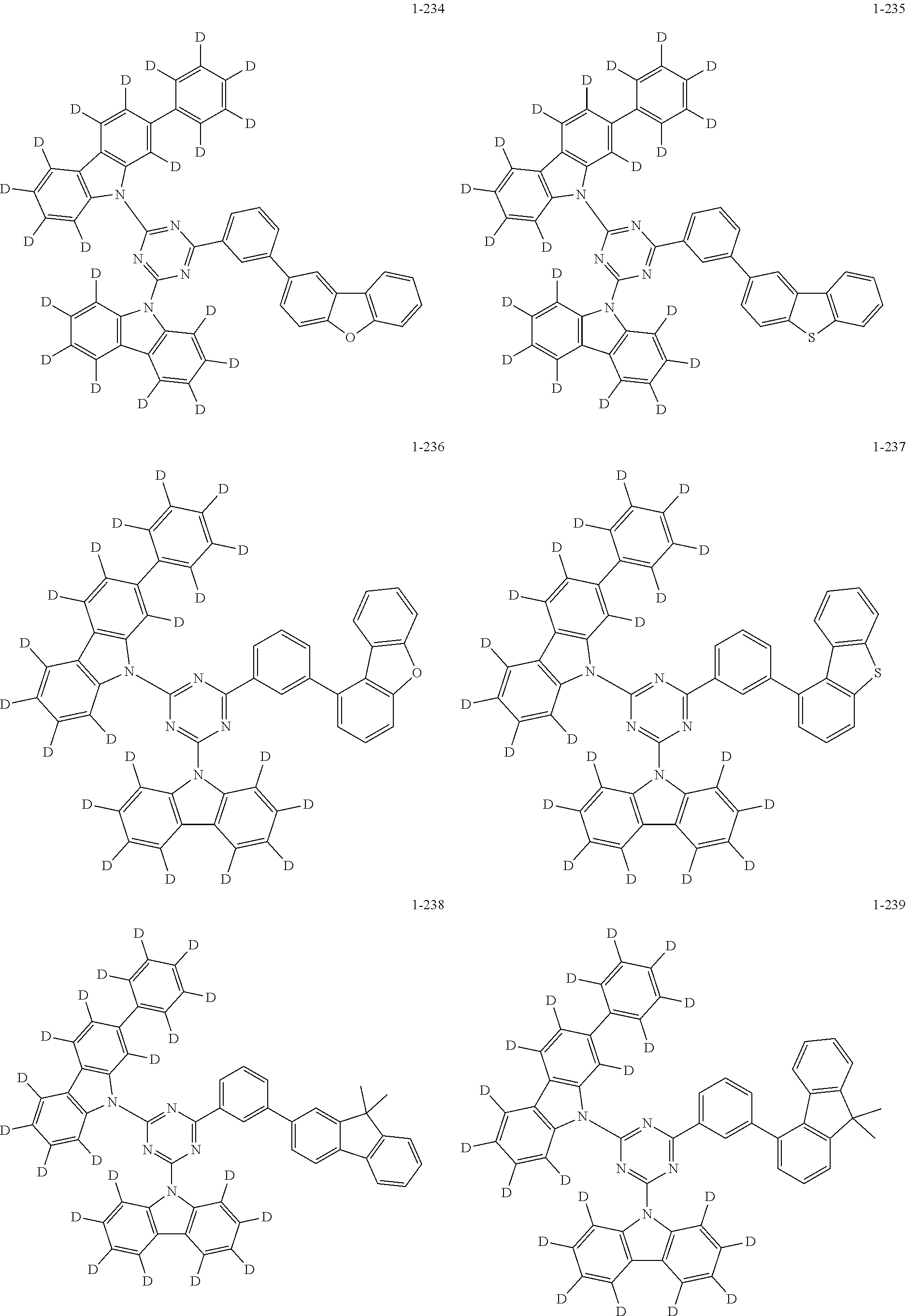

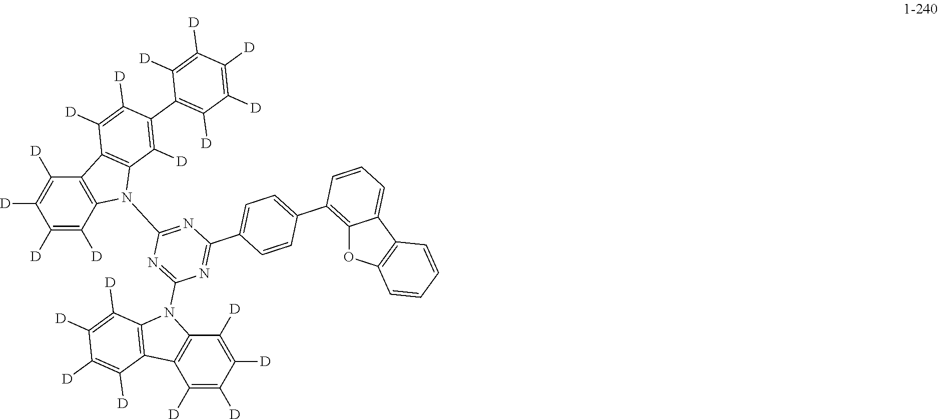 [SMILES: [2H]c1c([2H])c([2H])c(-c2c([2H])c([2H])c3c4c([2H])c([2H])c([2H])c([2H])c4n(-c4nc(-c5ccc(-c6cccc7c6oc6ccccc67)cc5)nc(-n5c6c([2H])c([2H])c([2H])c([2H])c6c6c([2H])c([2H])c([2H])c([2H])c65)n4)c3c2[2H])c([2H])c1[2H]]

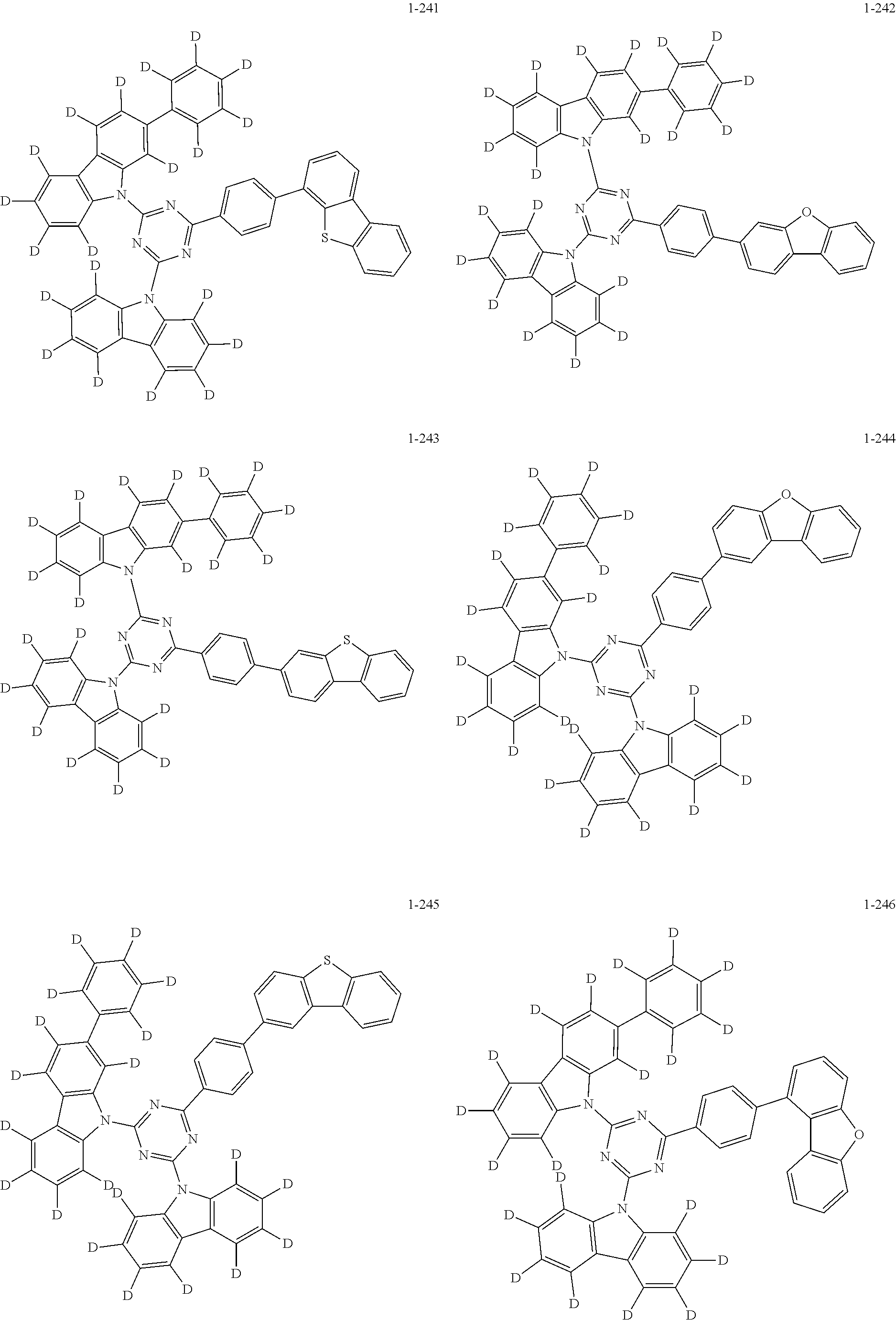

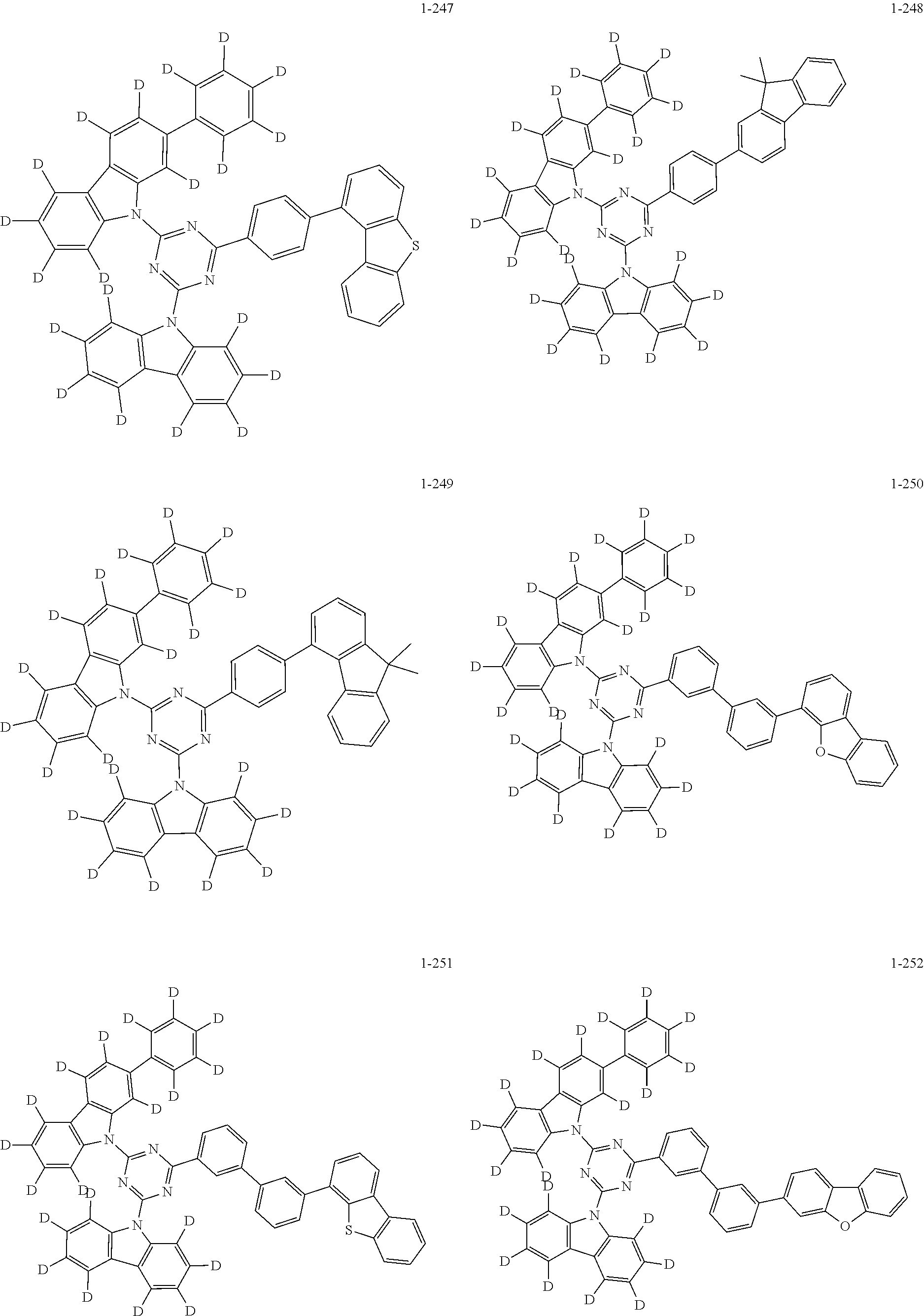

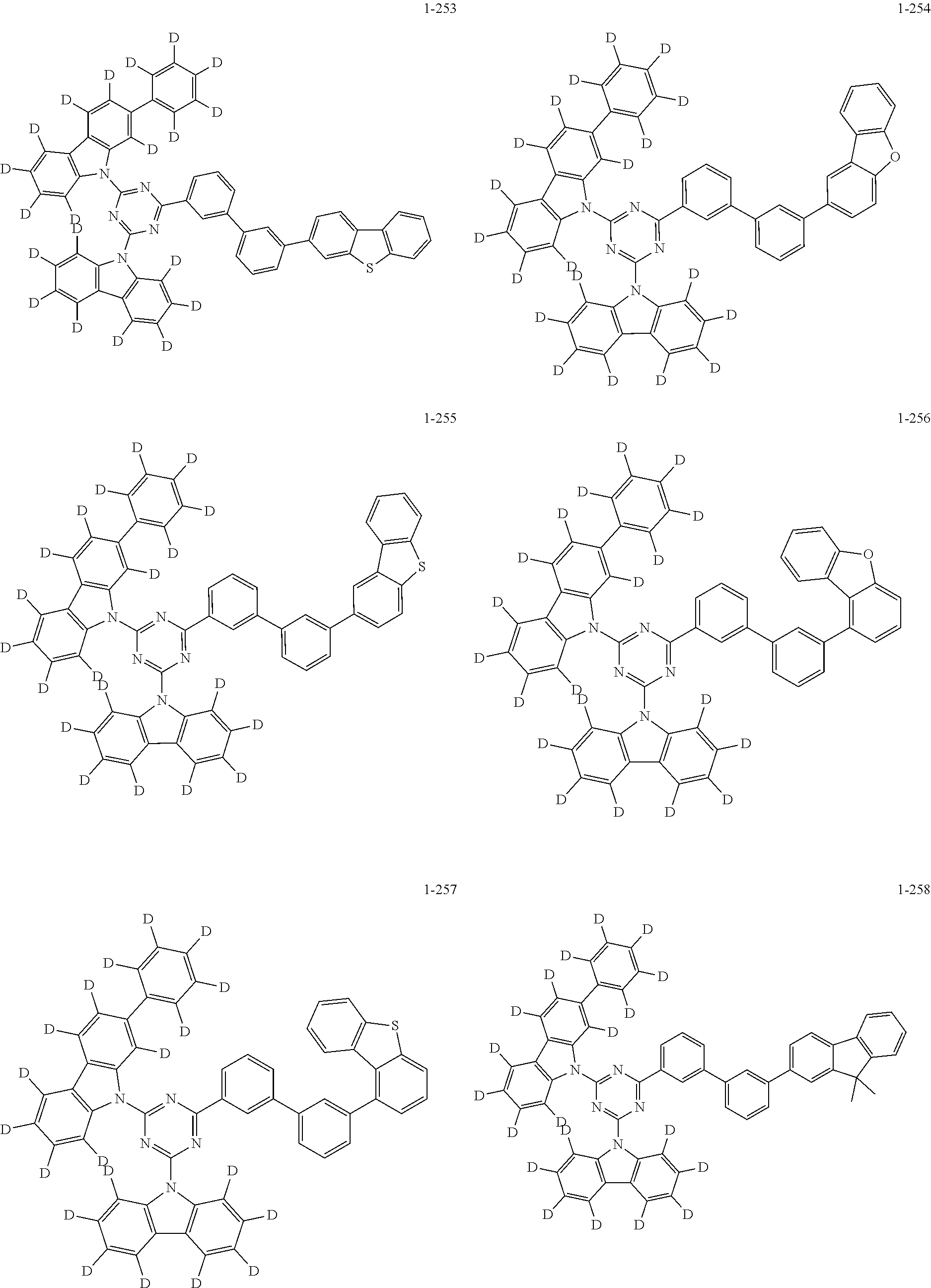

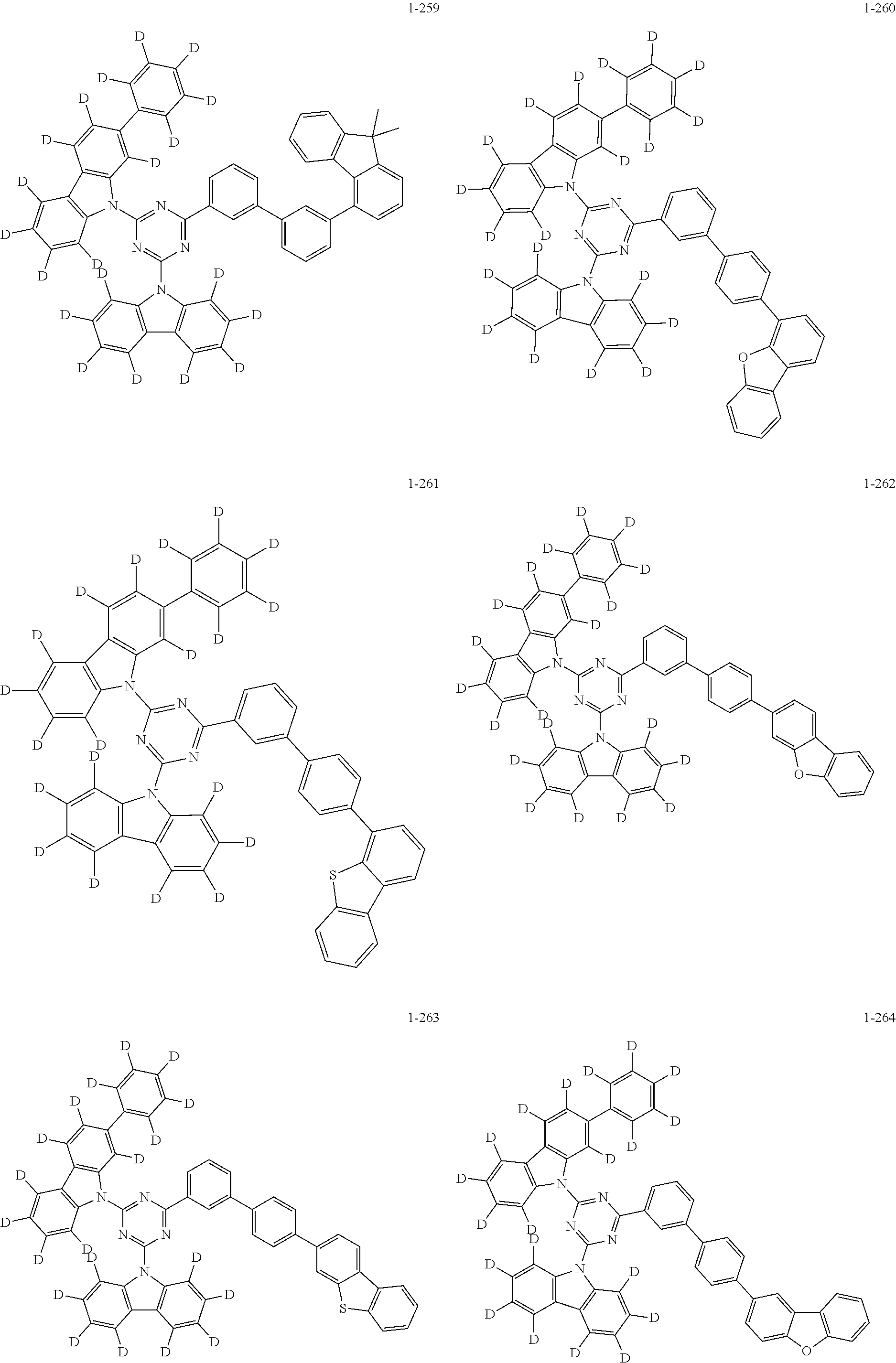

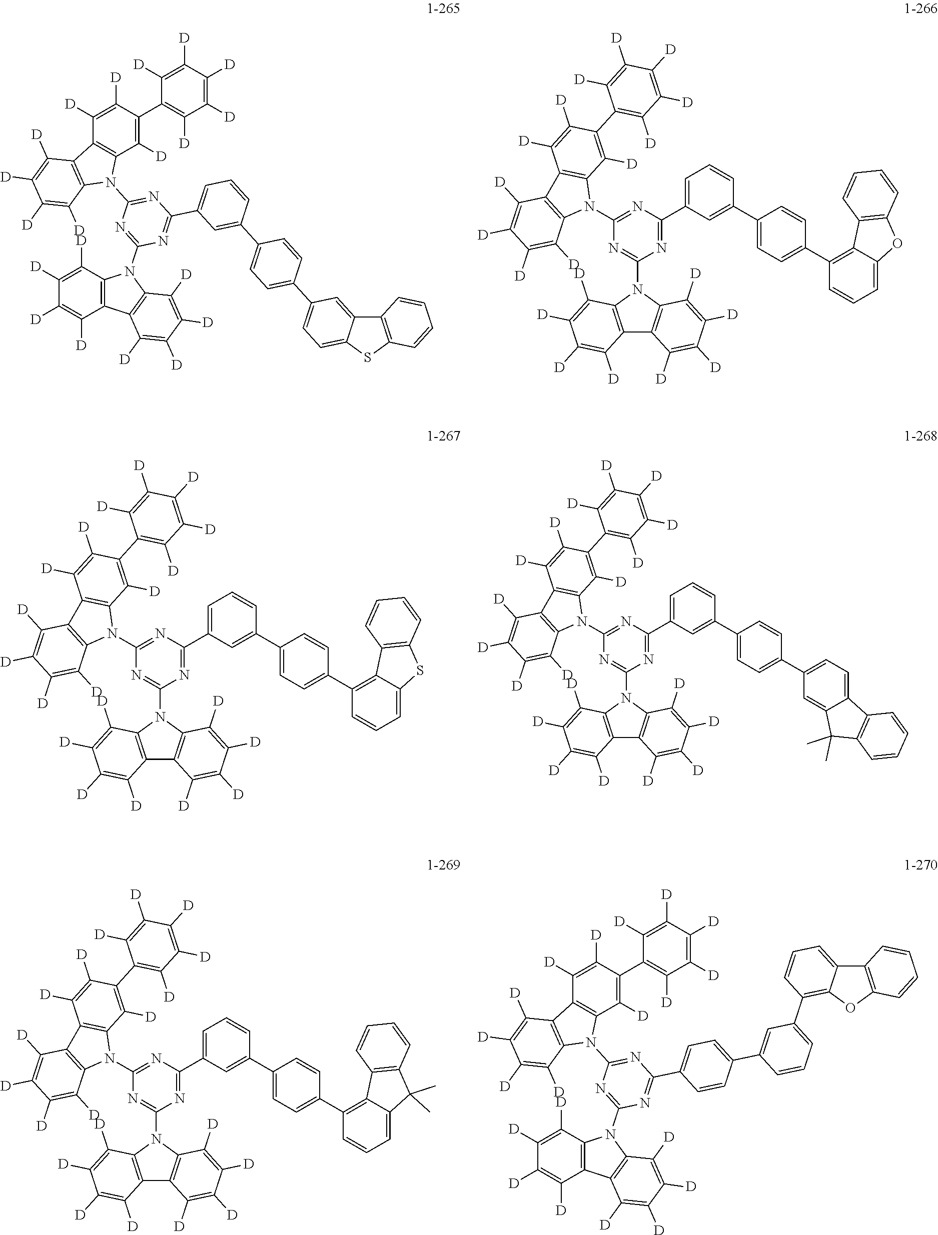

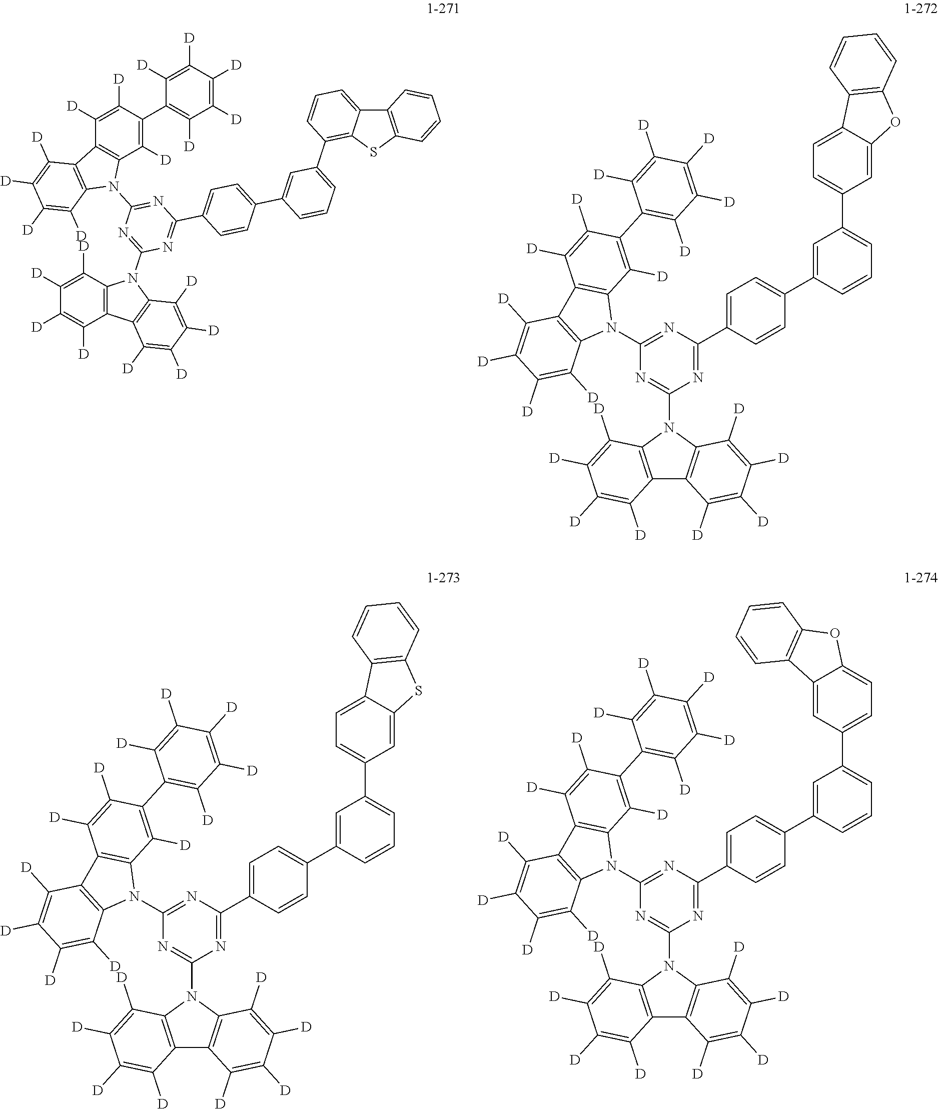

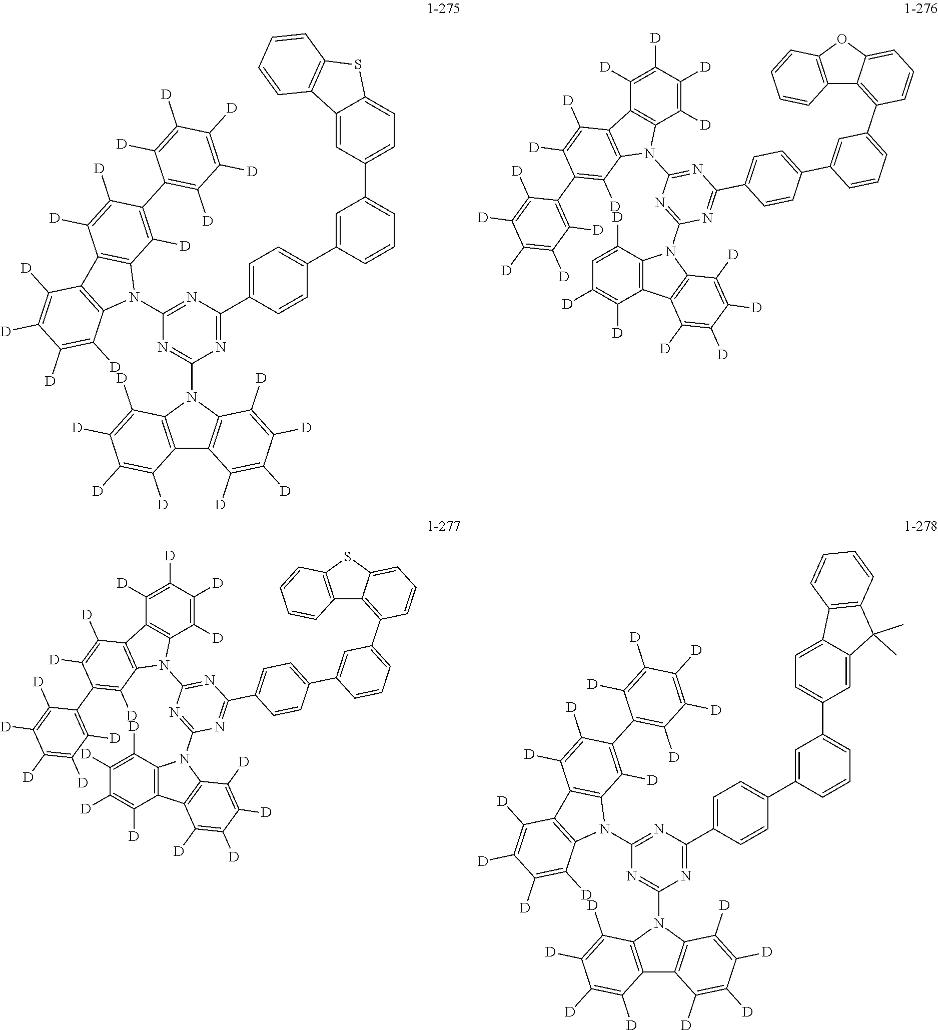

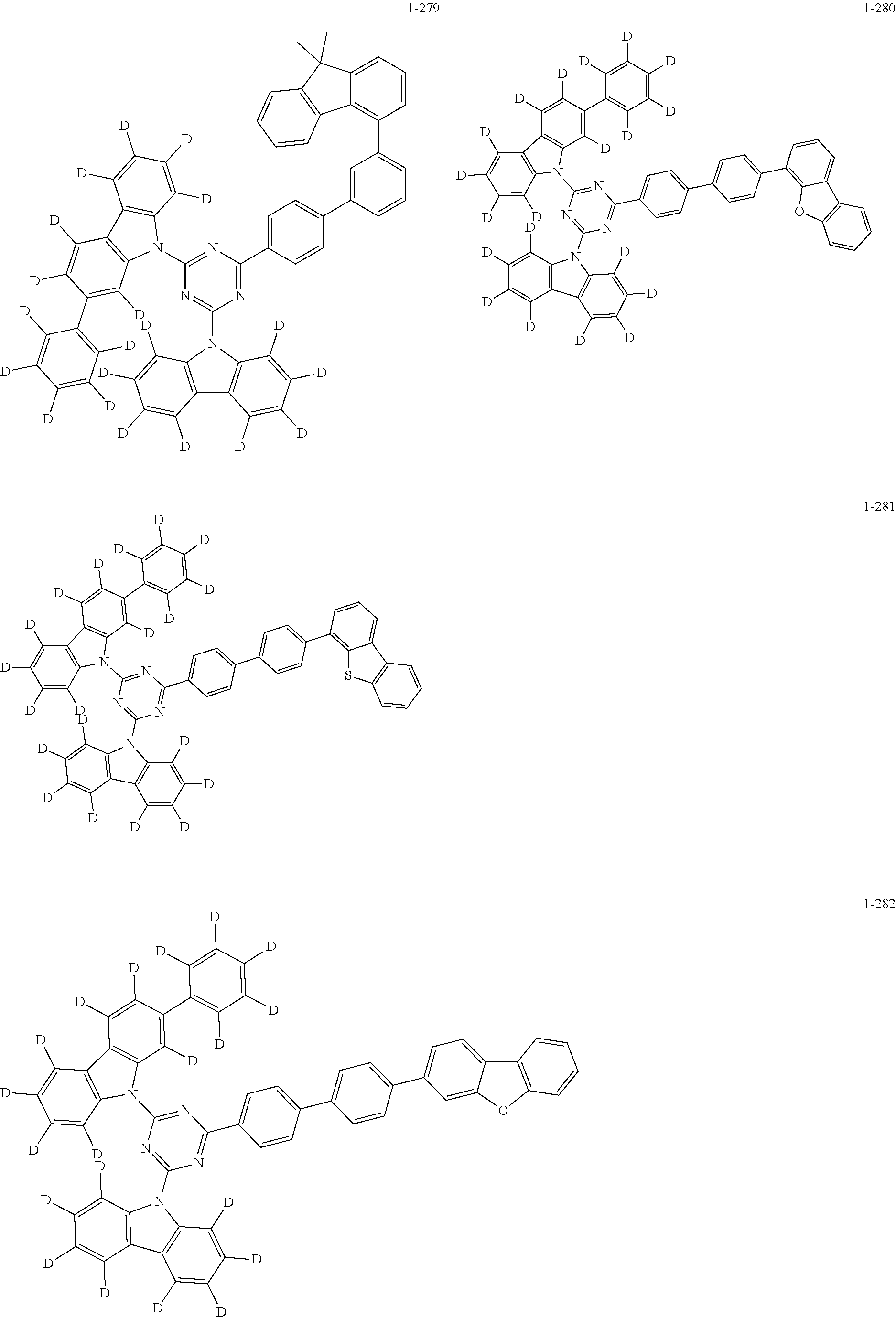

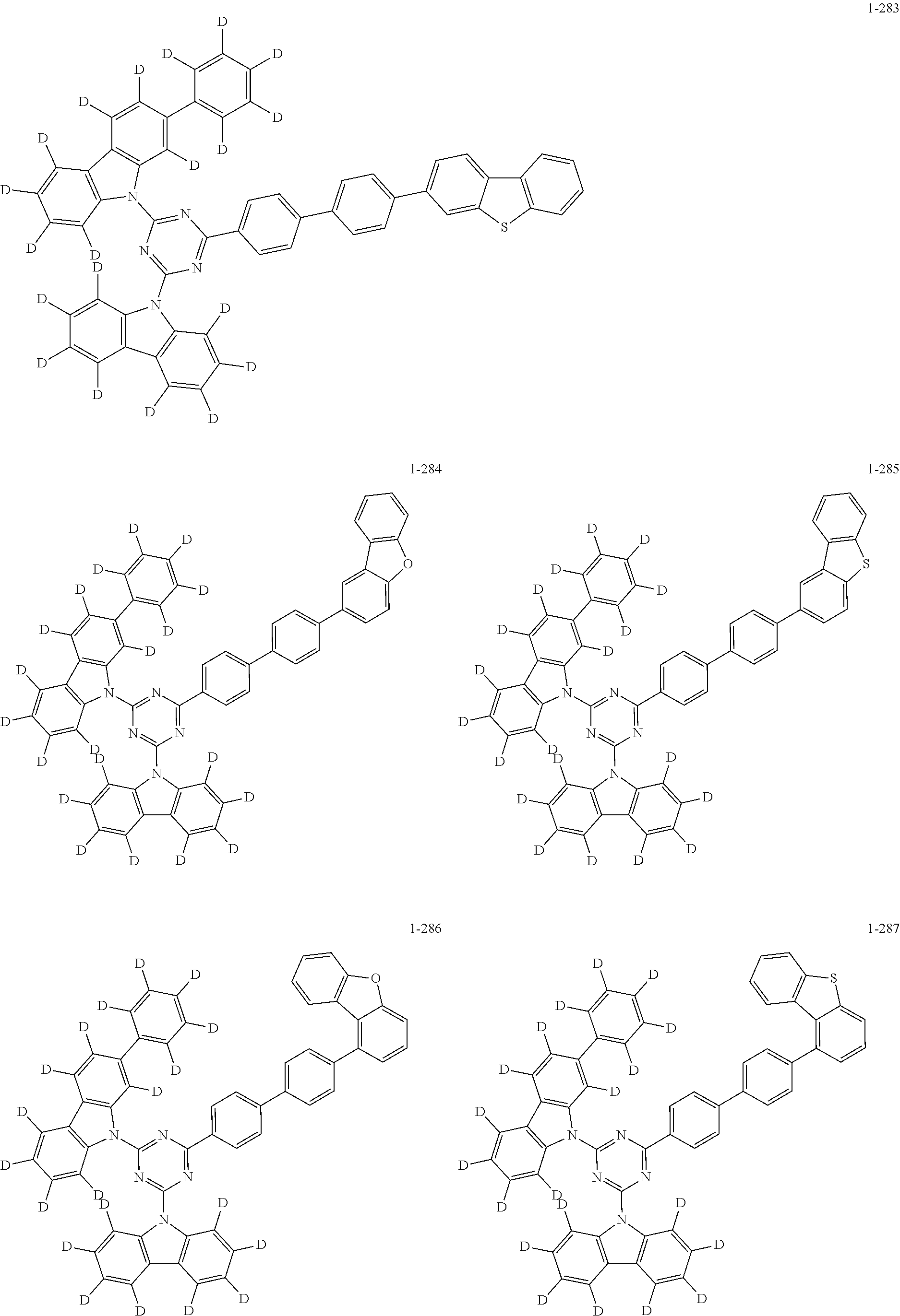

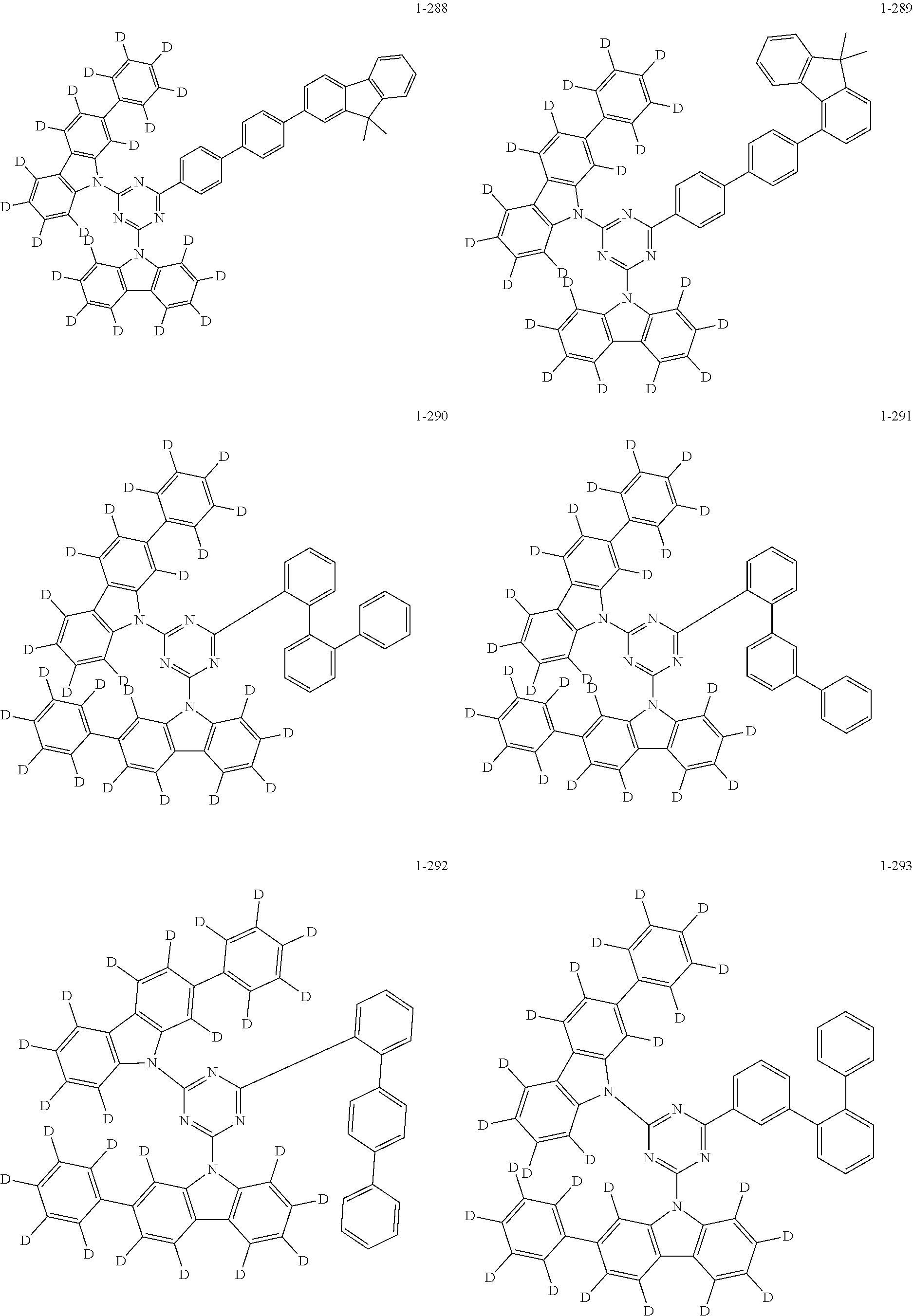

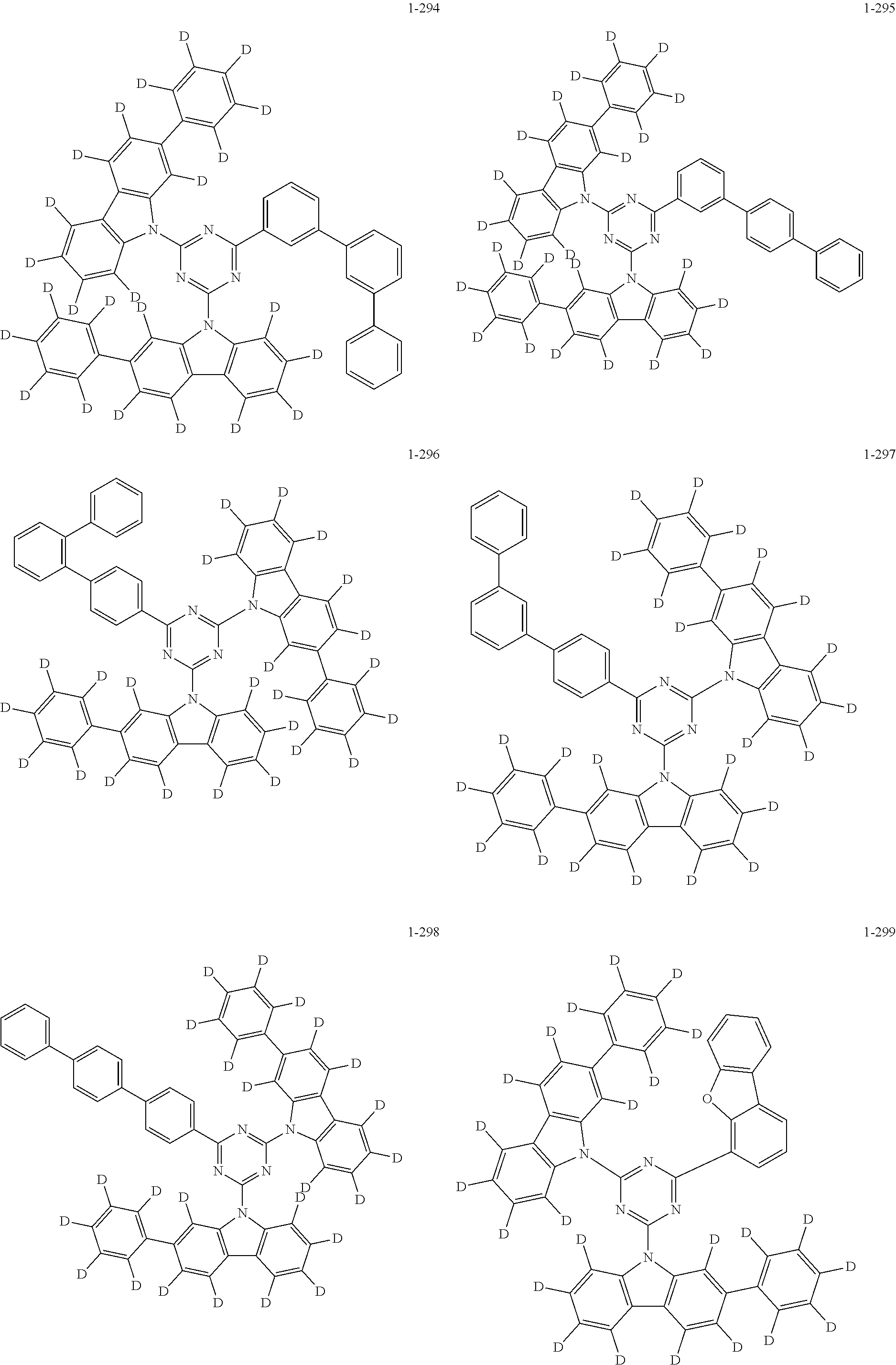

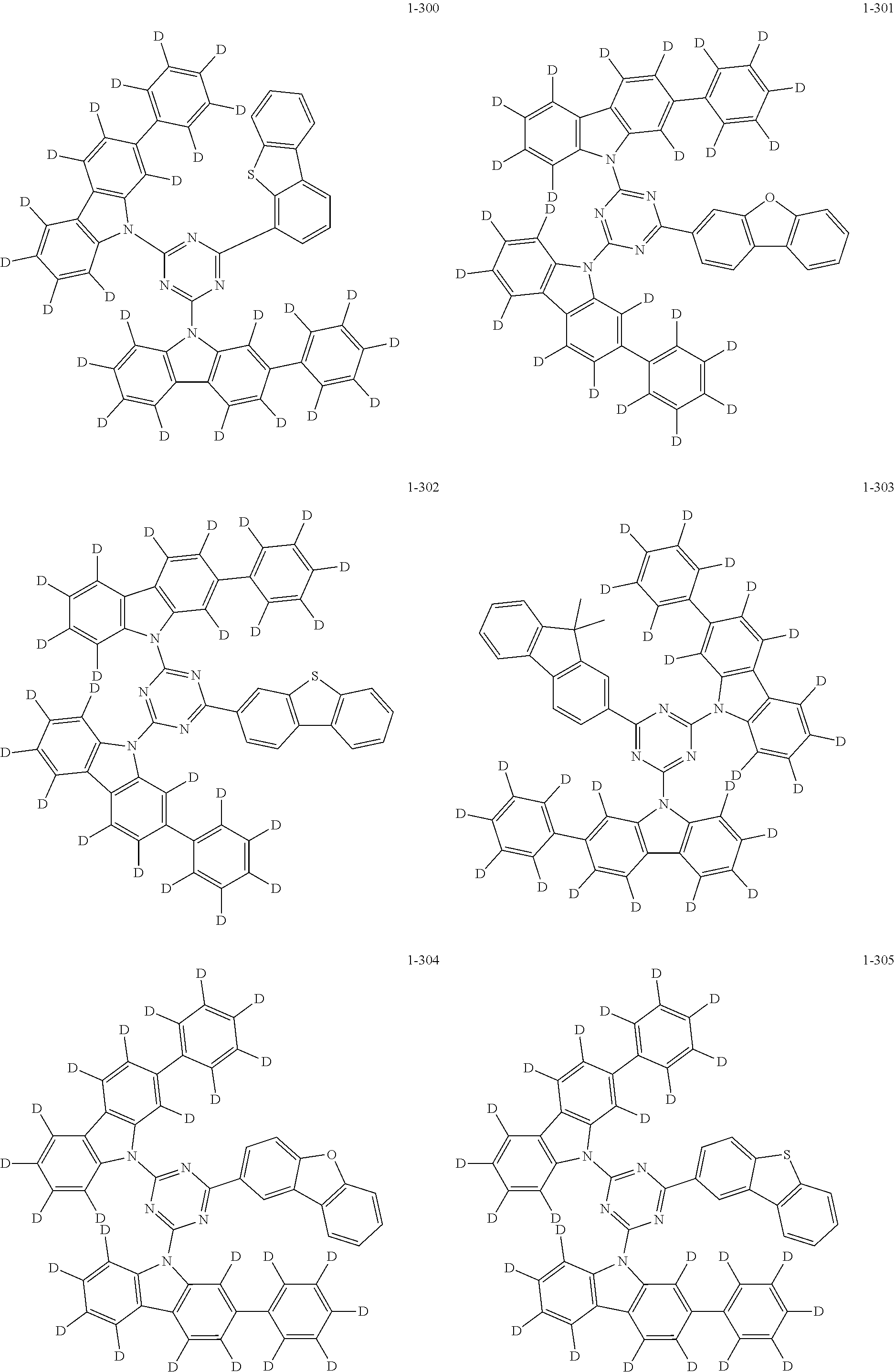

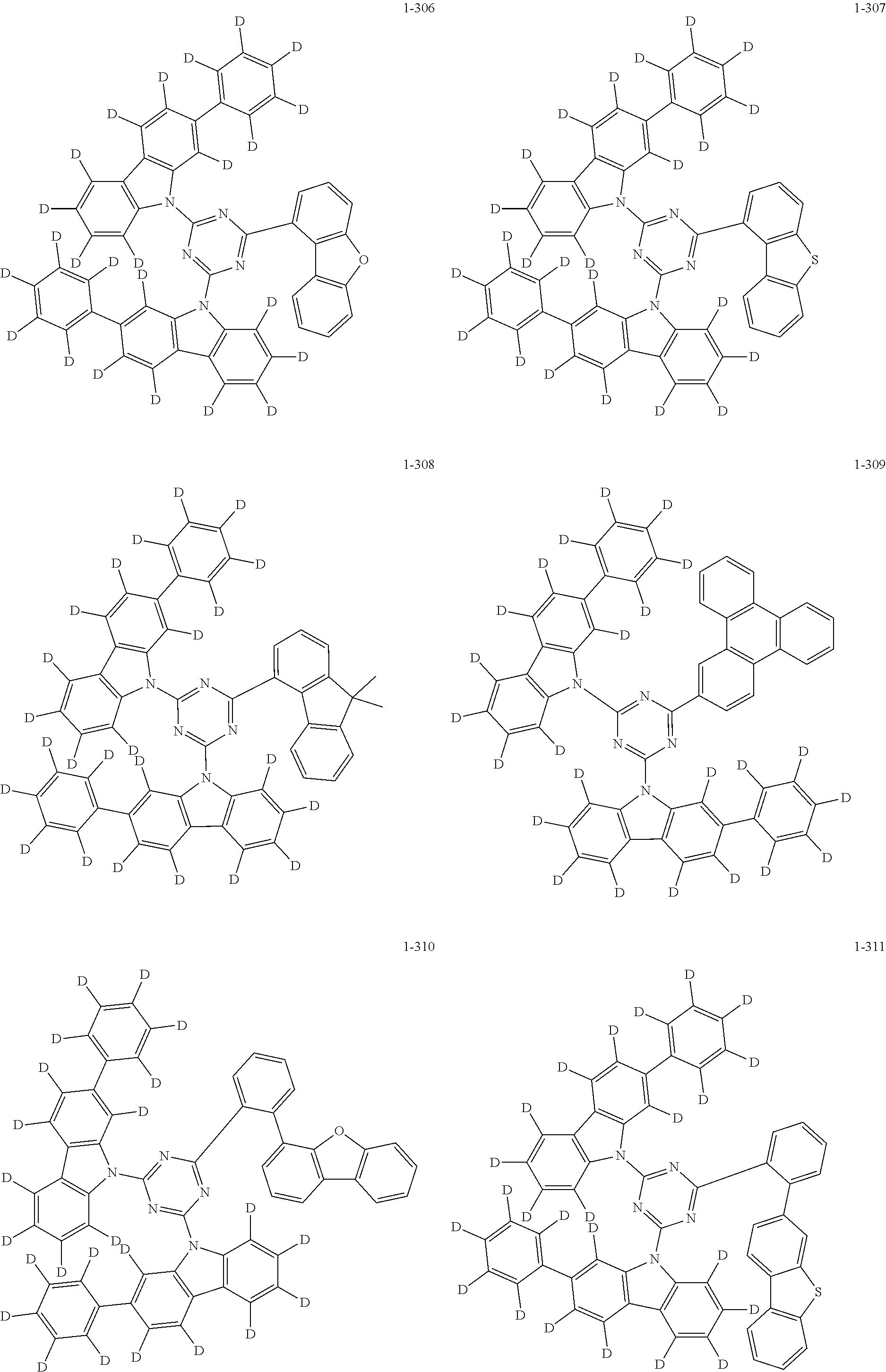

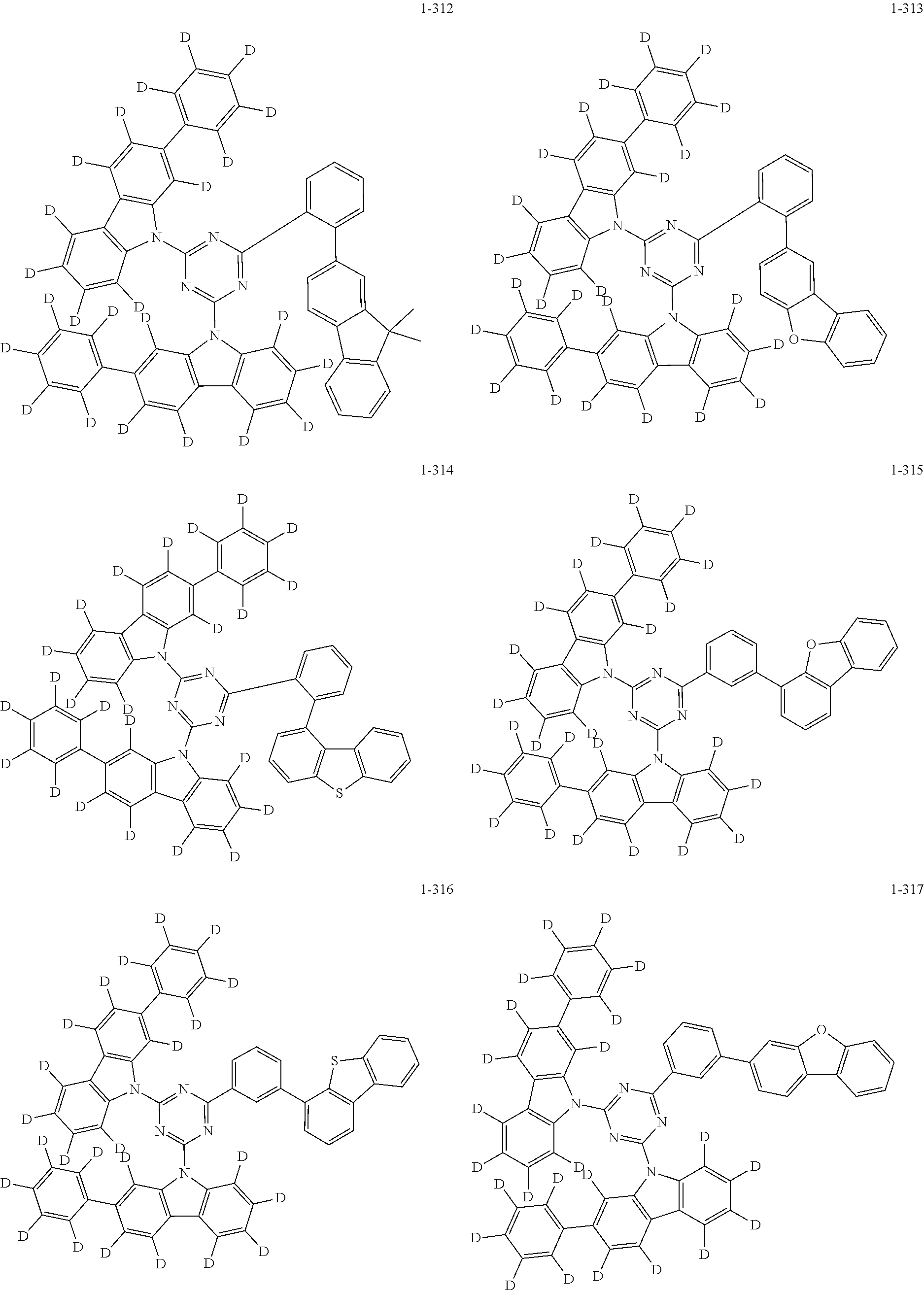

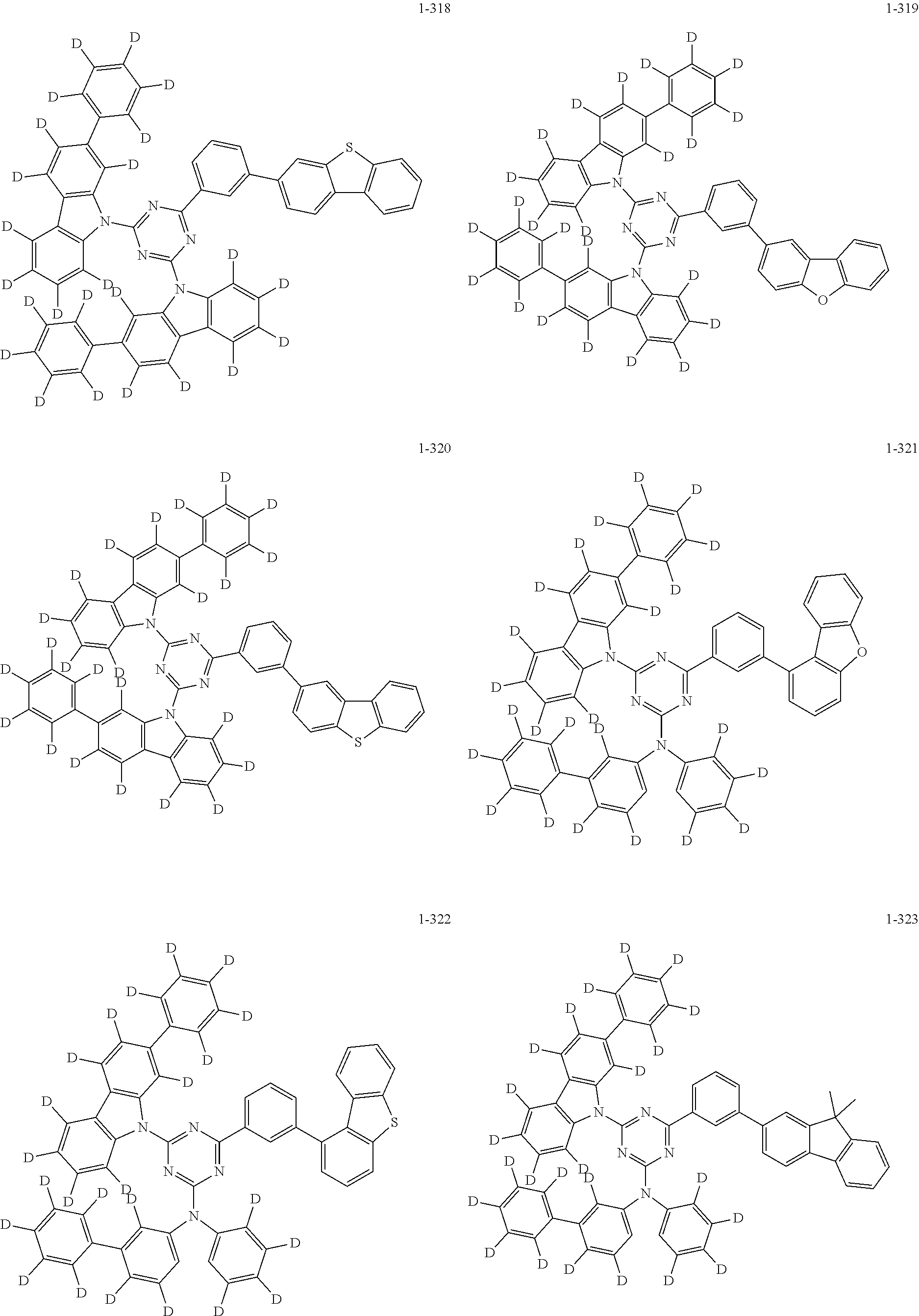

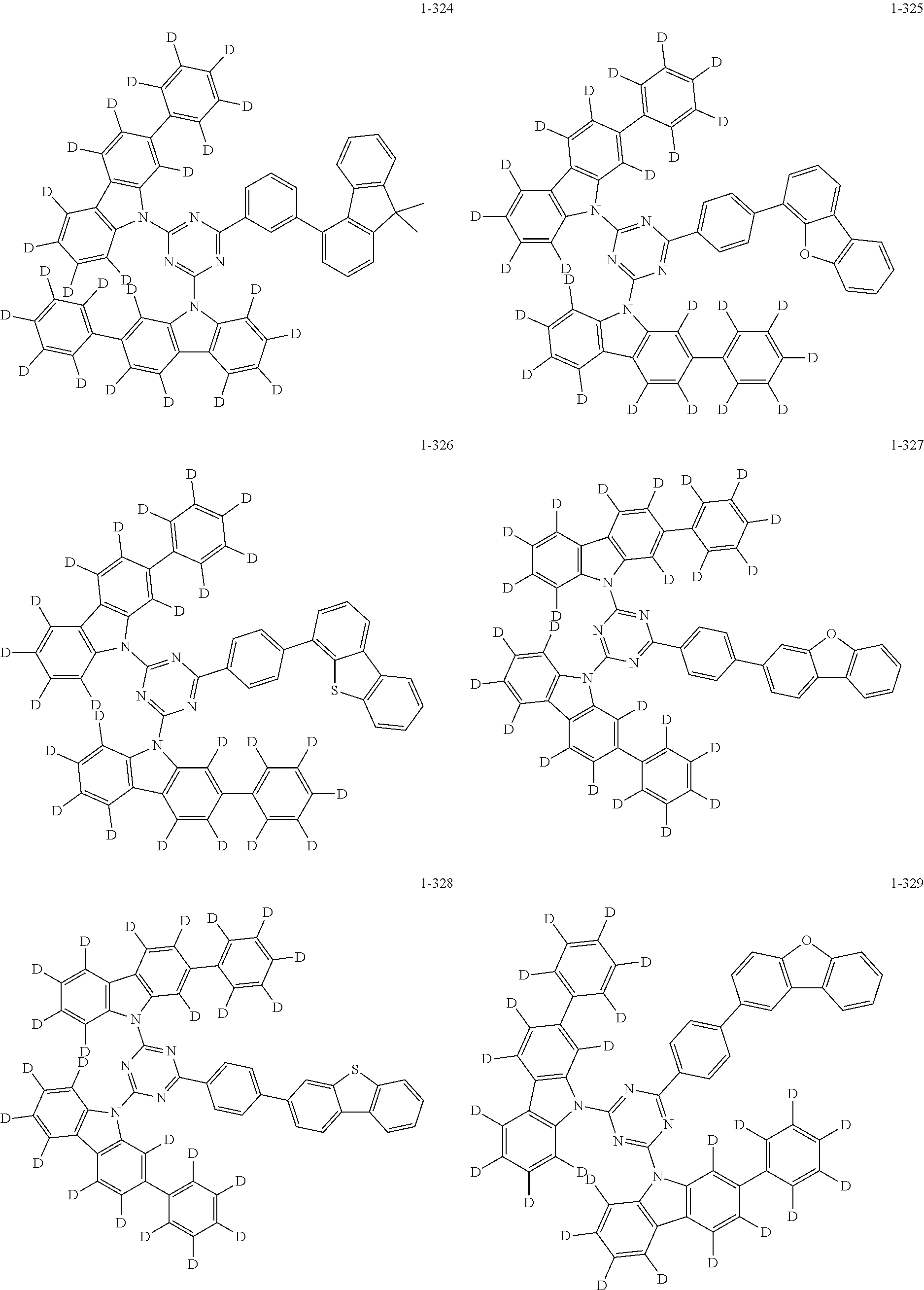

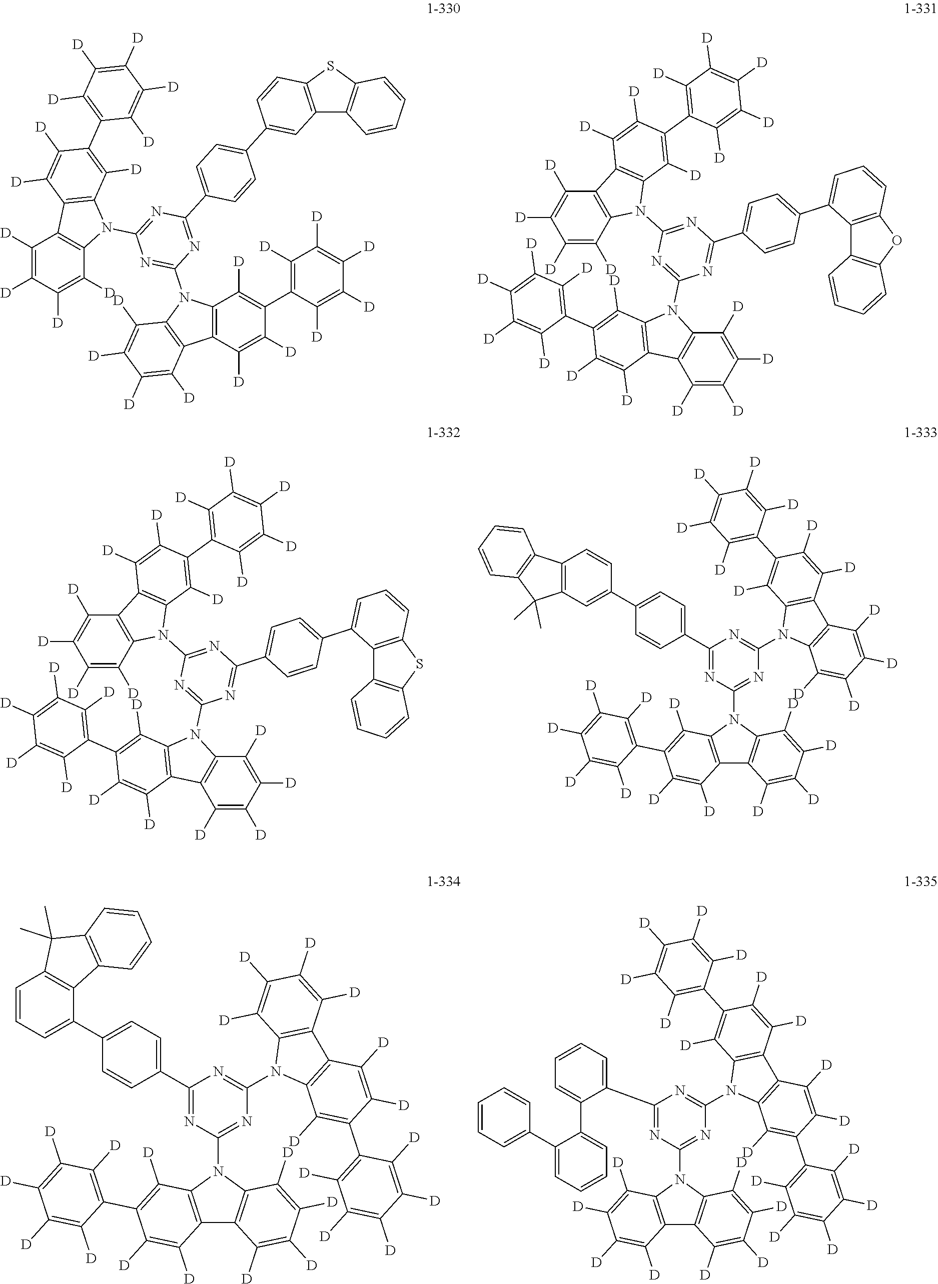

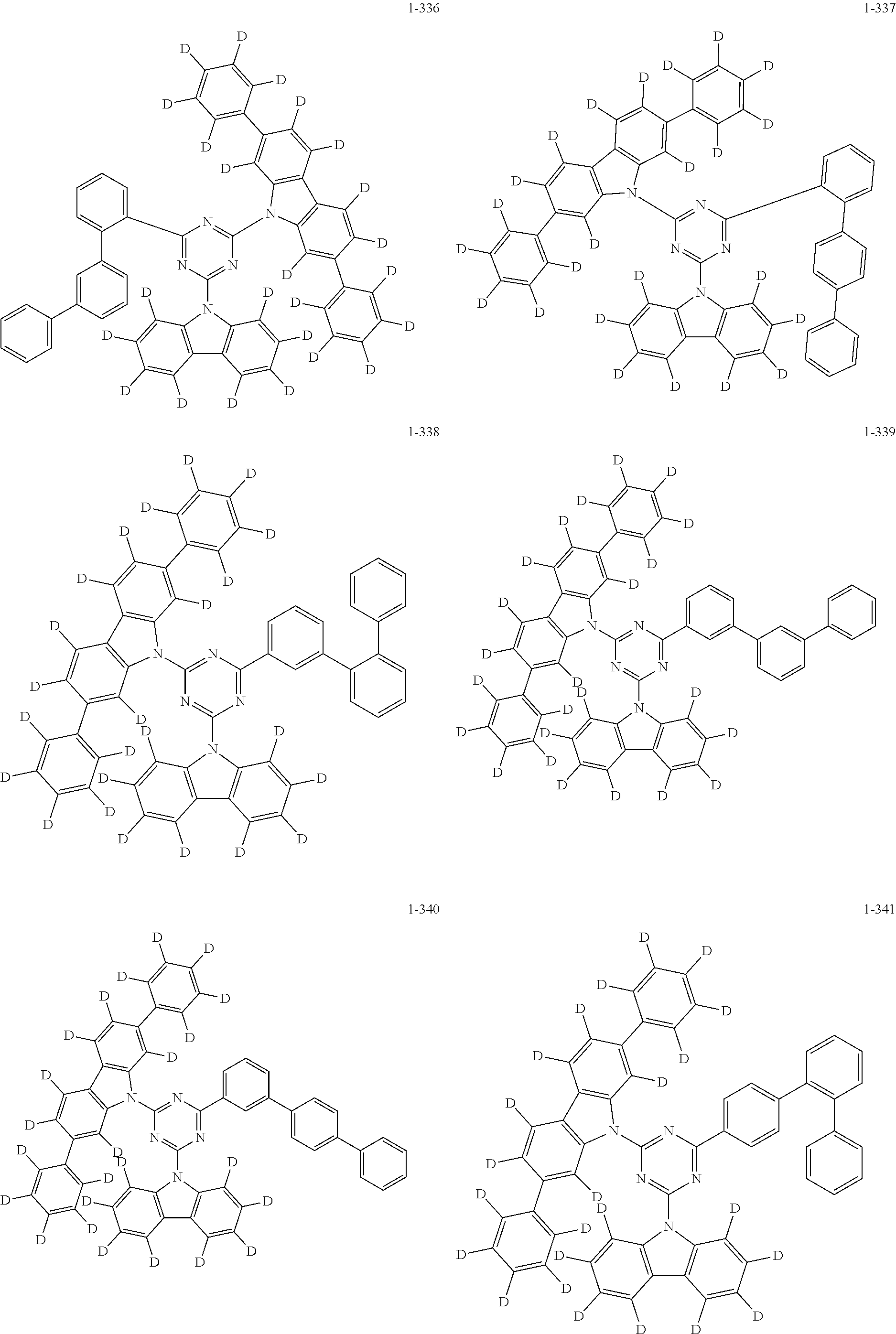

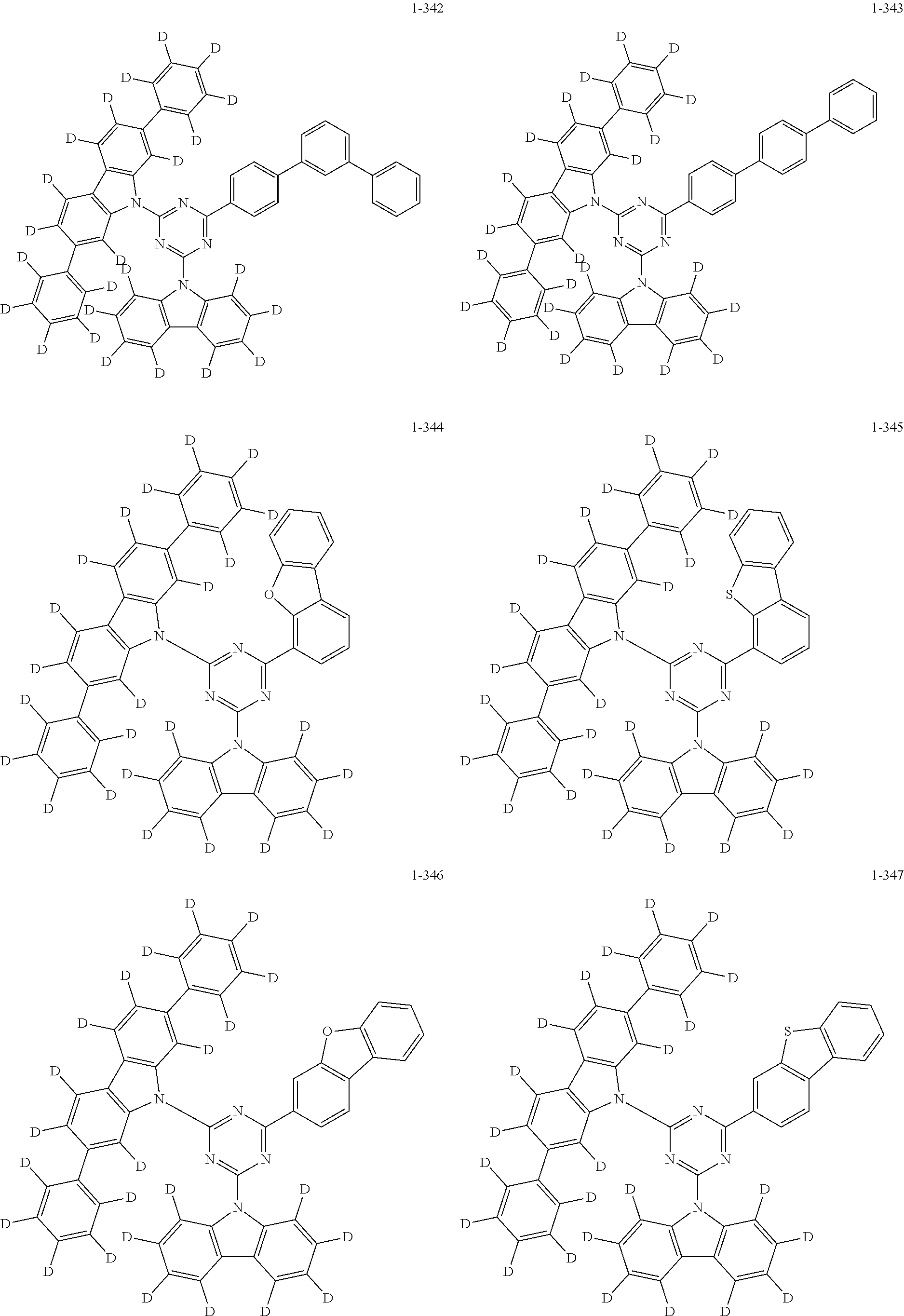

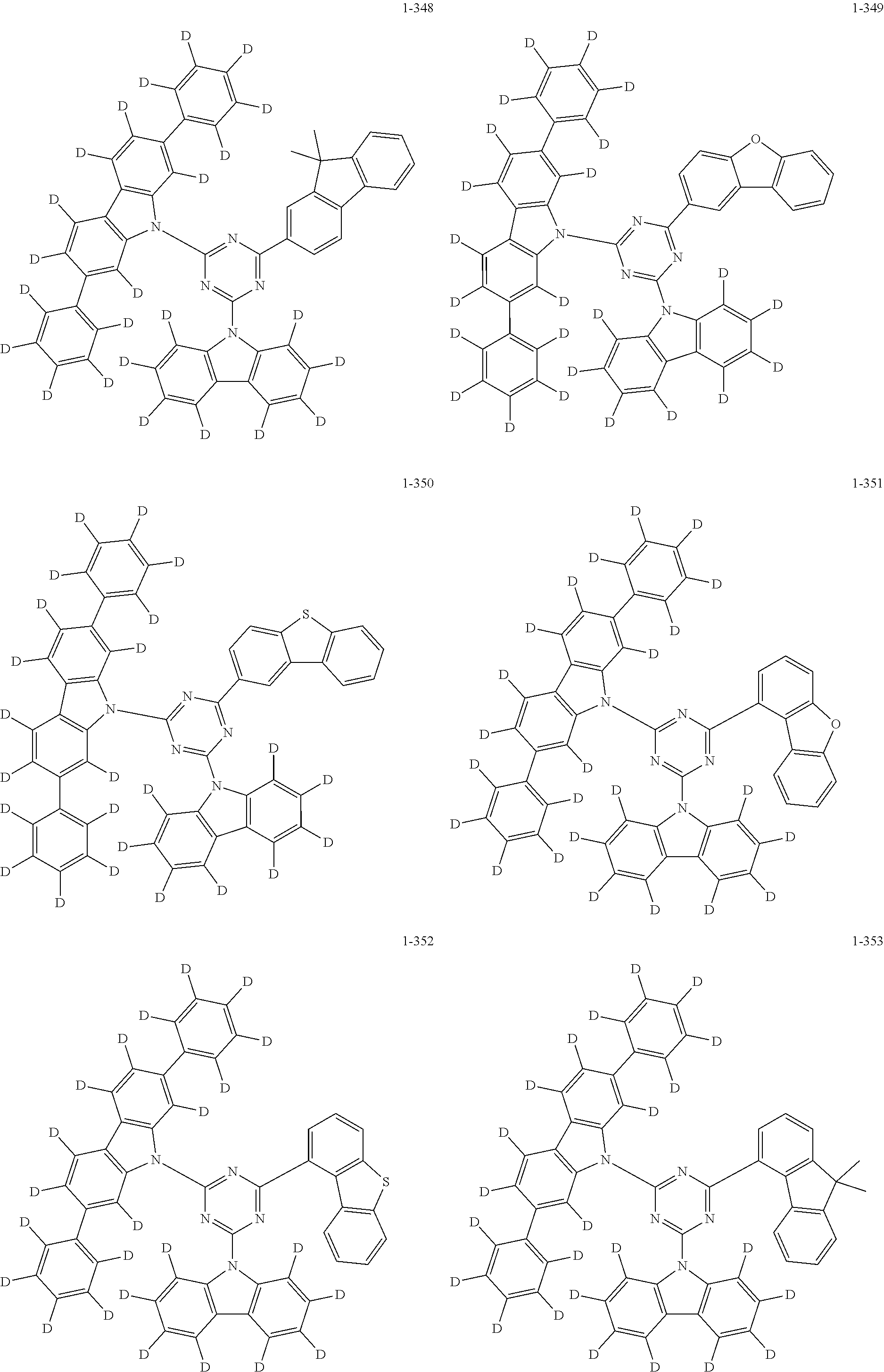

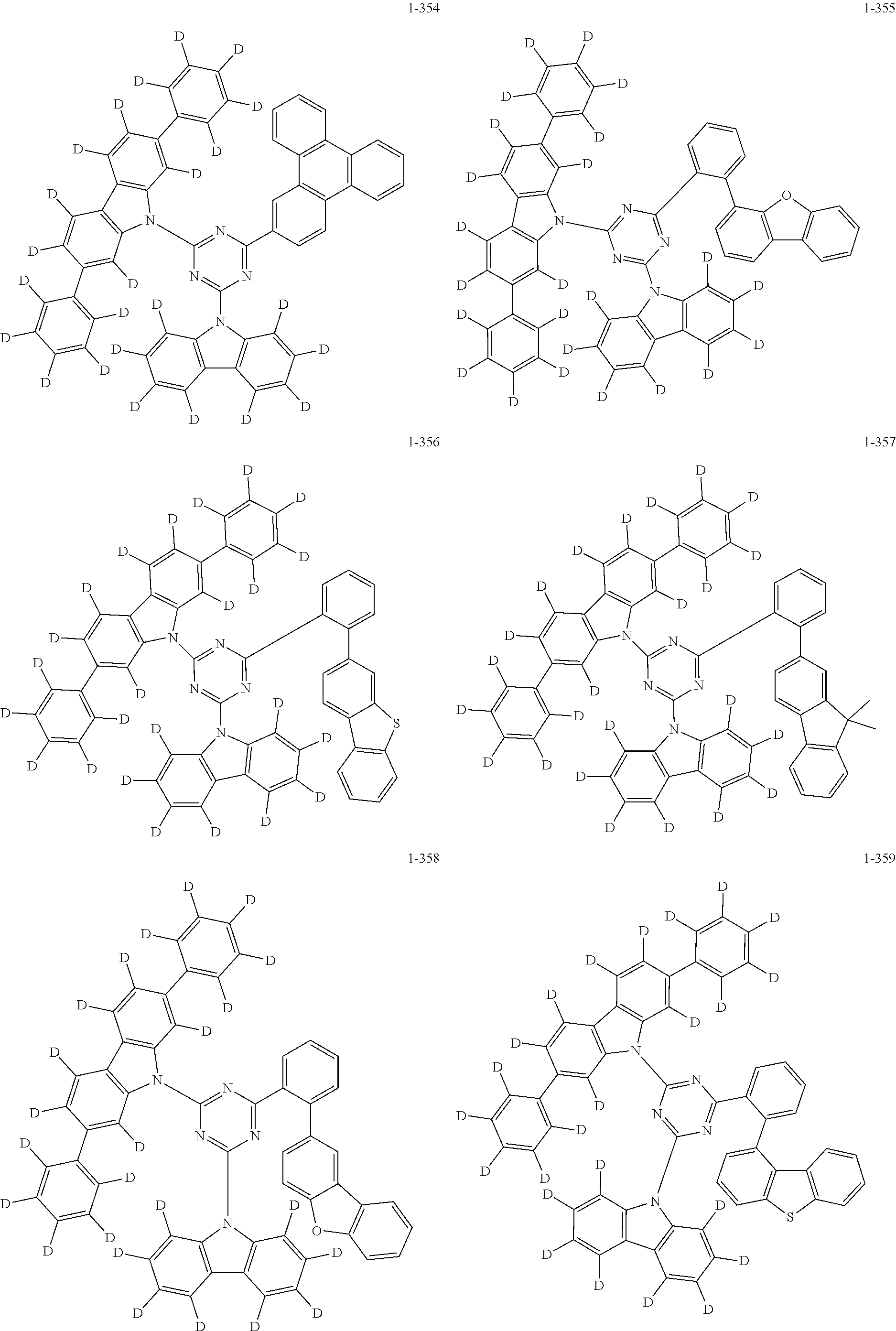

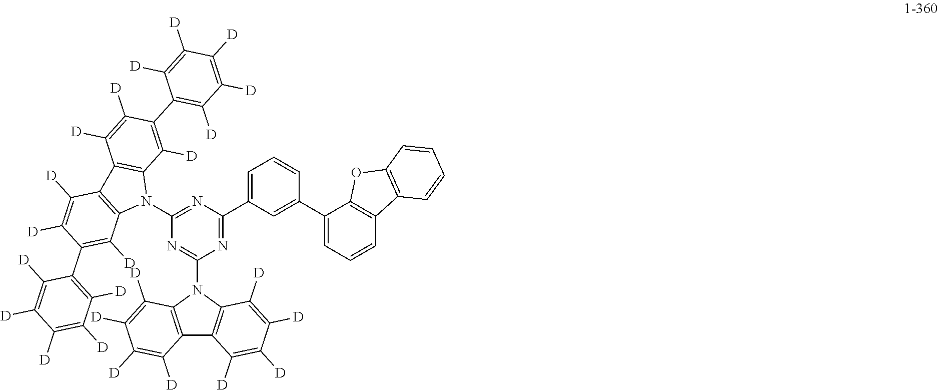 [SMILES: [2H]c1c([2H])c([2H])c(-c2c([2H])c([2H])c3c4c([2H])c([2H])c(-c5c([2H])c([2H])c([2H])c([2H])c5[2H])c([2H])c4n(-c4nc(-c5cccc(-c6cccc7c6oc6ccccc67)c5)nc(-n5c6c([2H])c([2H])c([2H])c([2H])c6c6c([2H])c([2H])c([2H])c([2H])c65)n4)c3c2[2H])c([2H])c1[2H]]

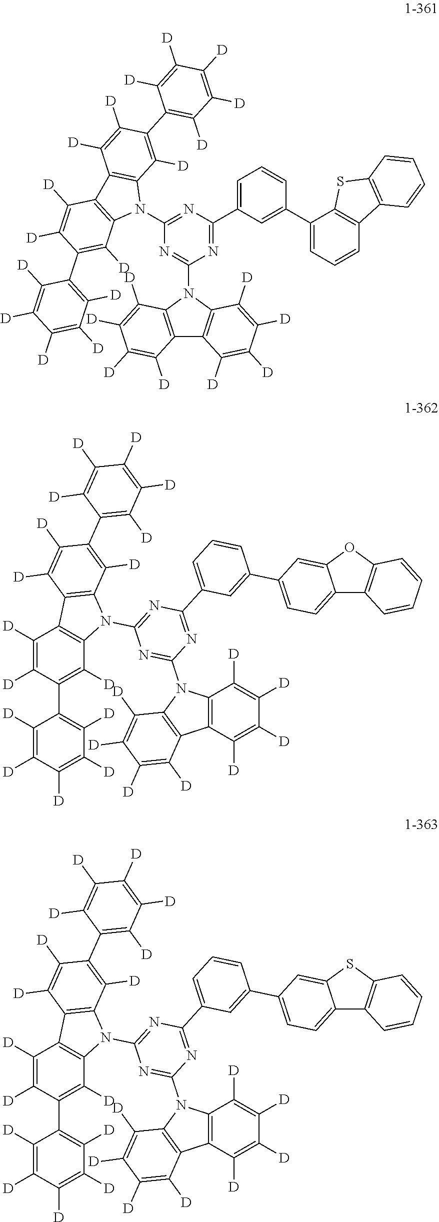

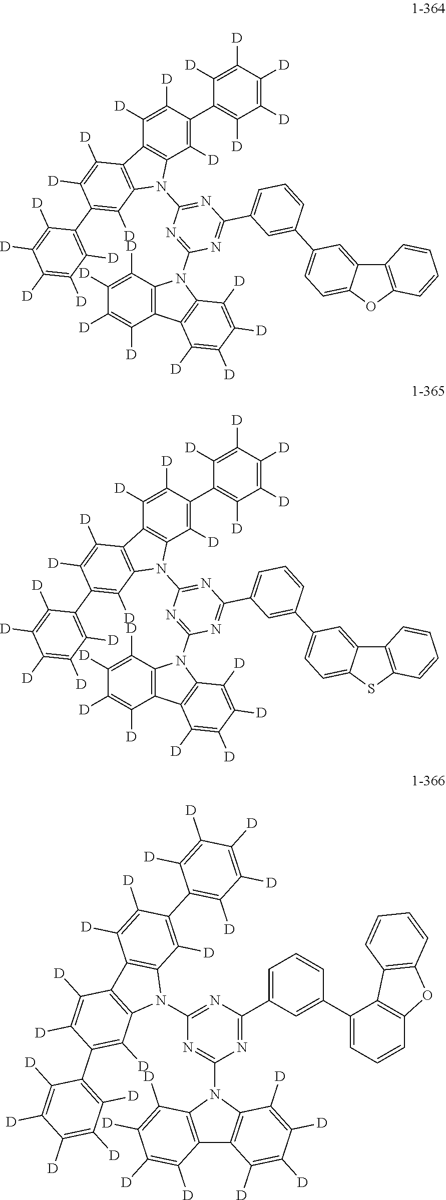

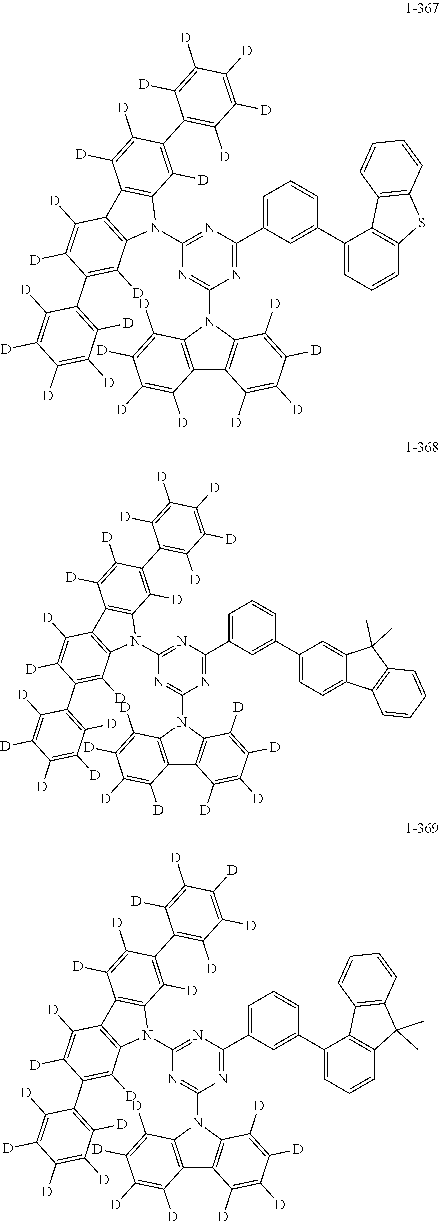

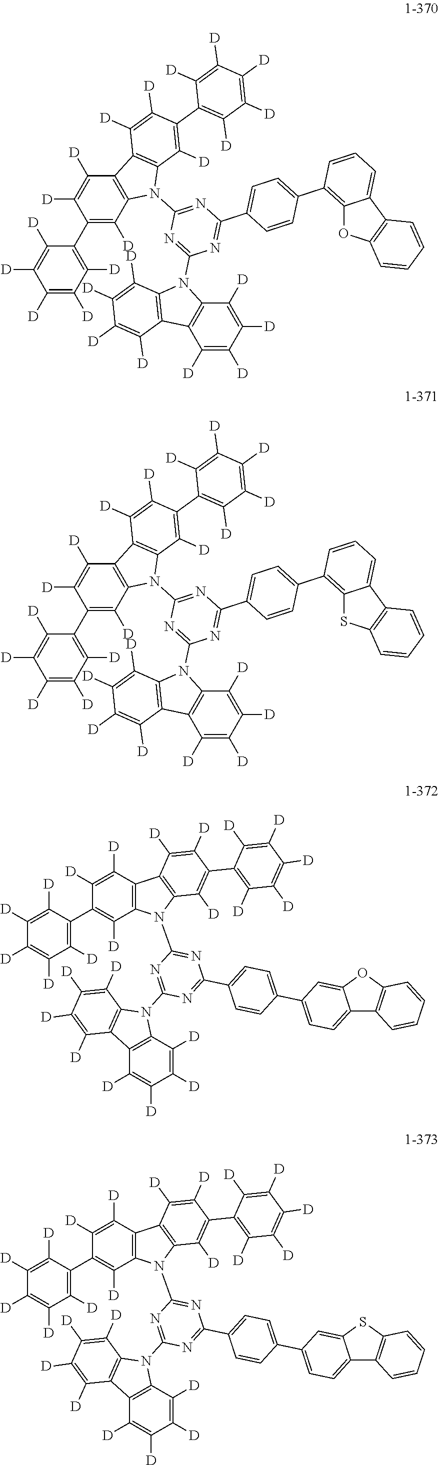

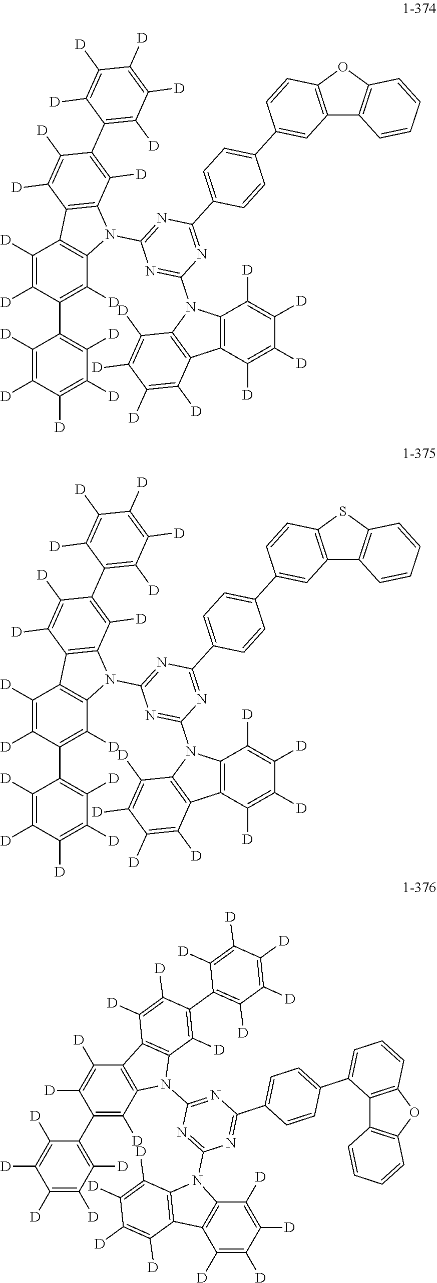

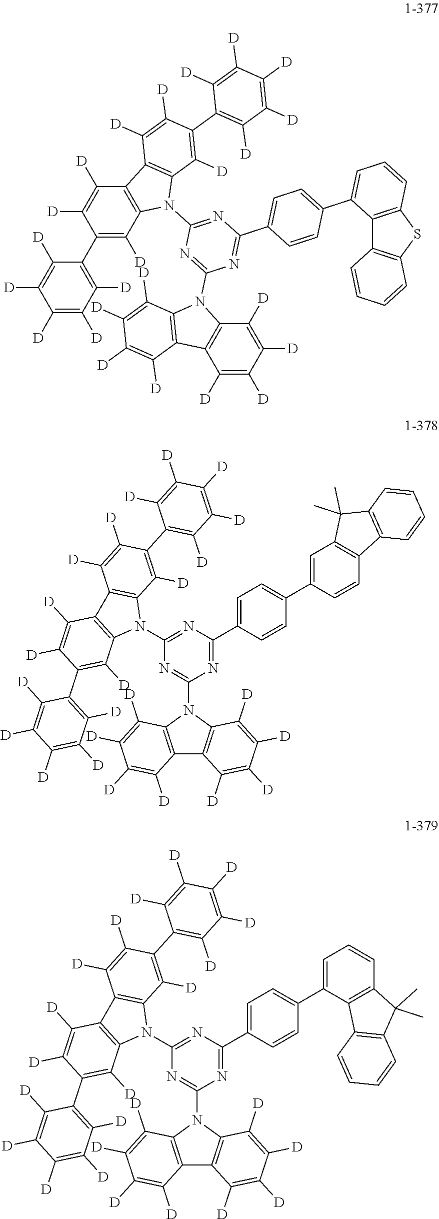

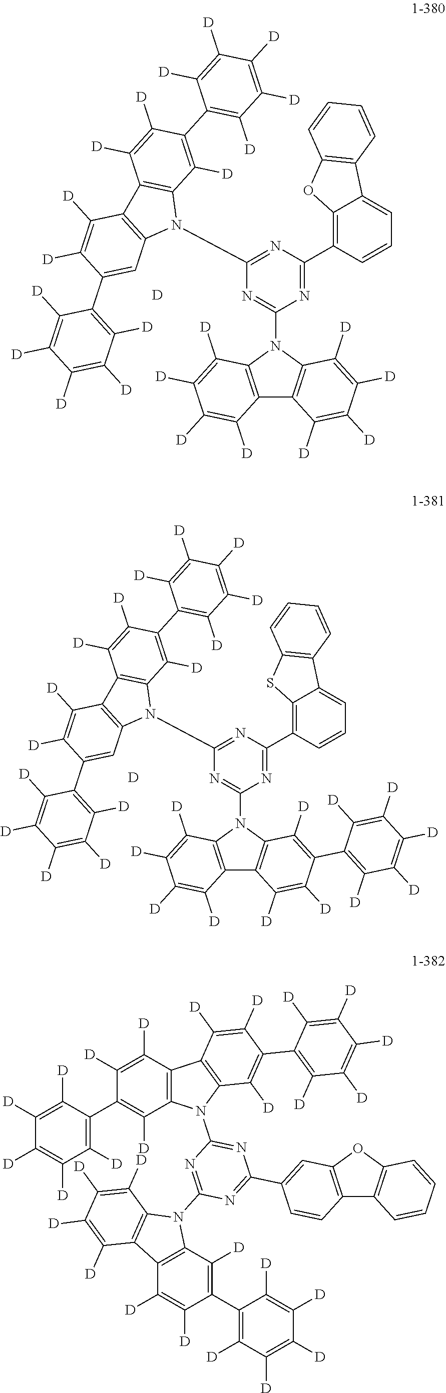

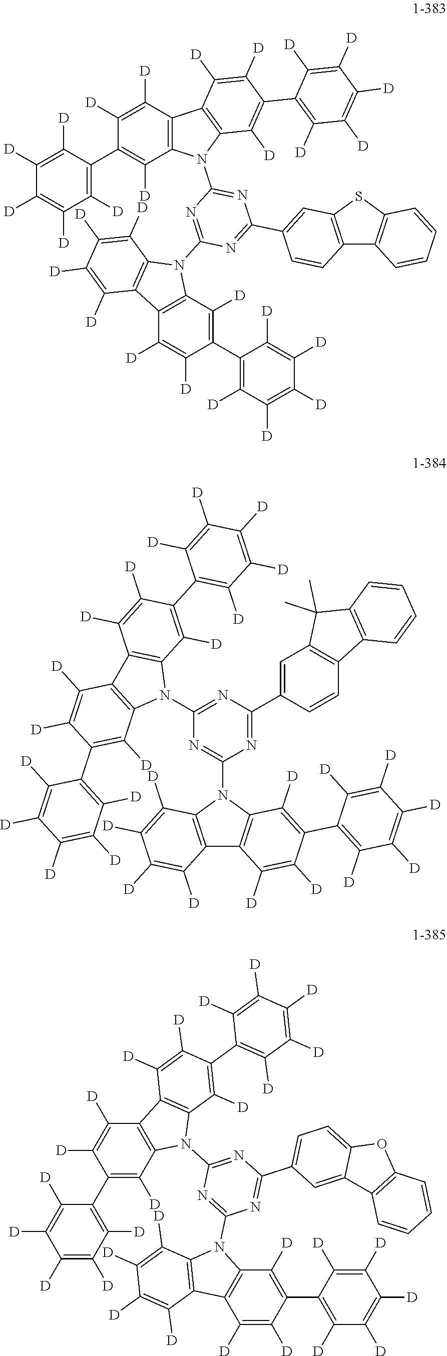

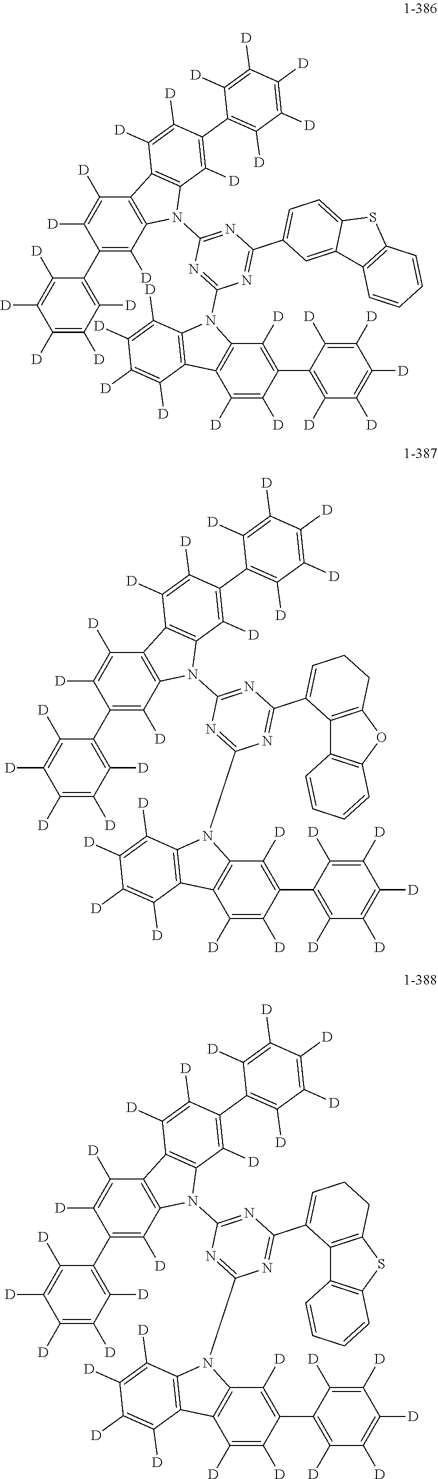

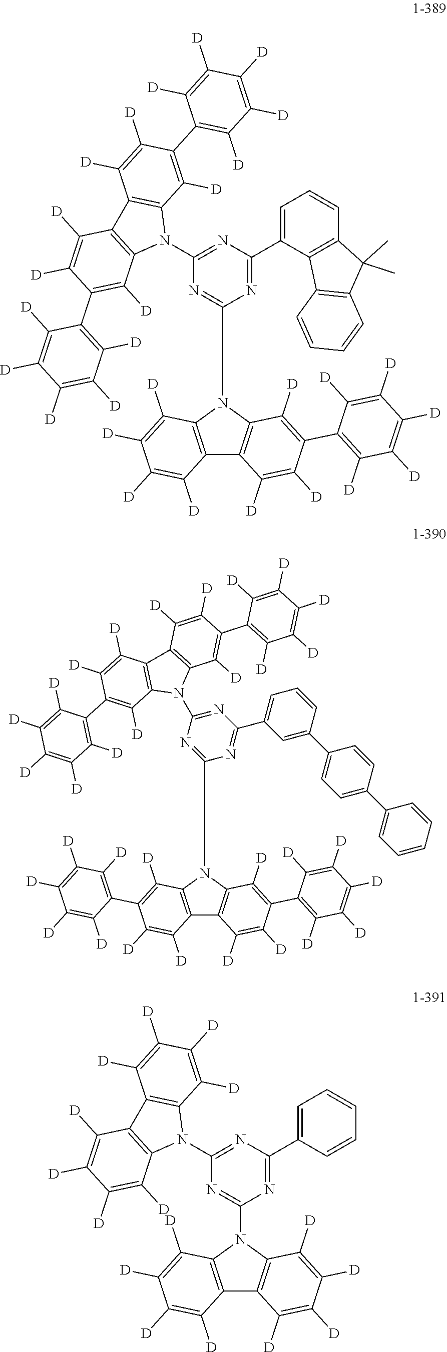

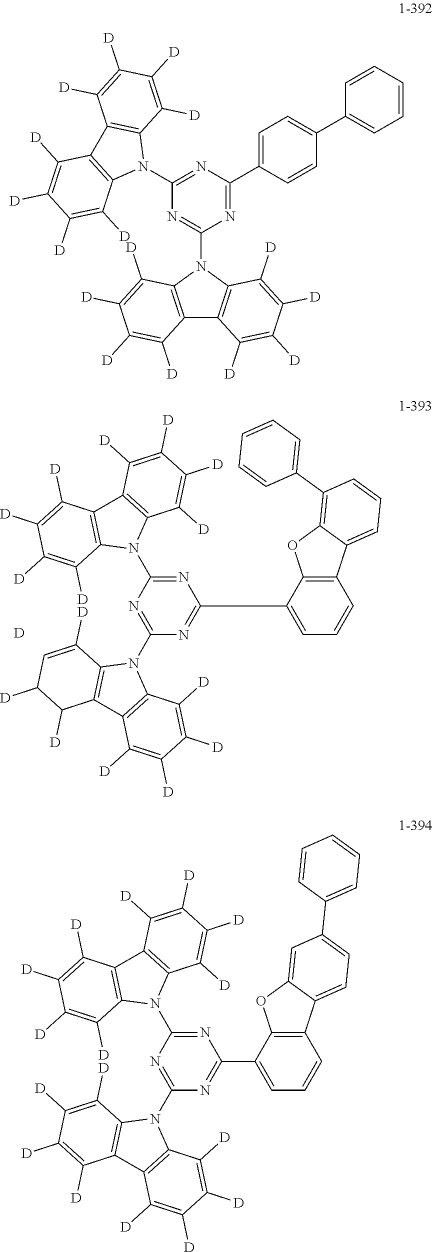

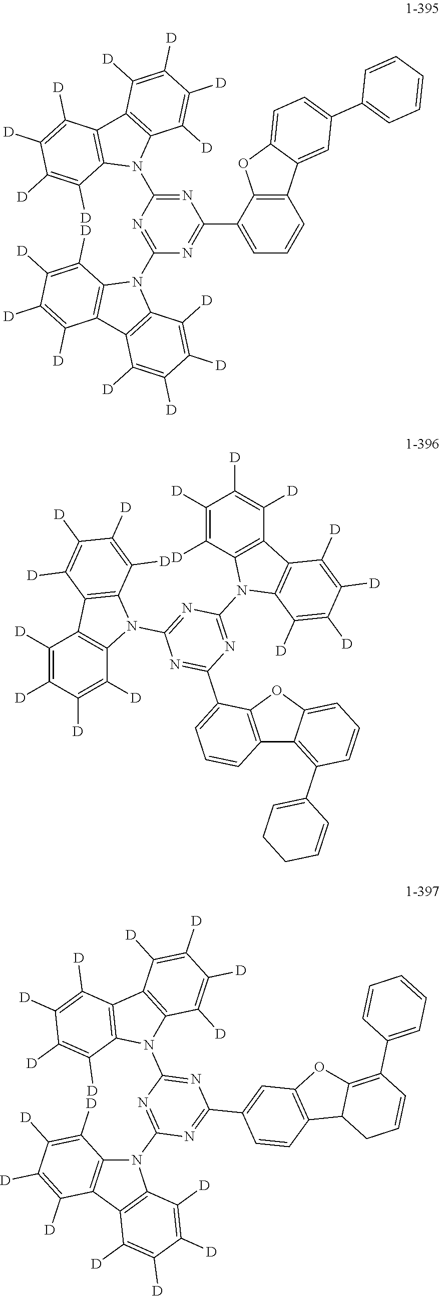

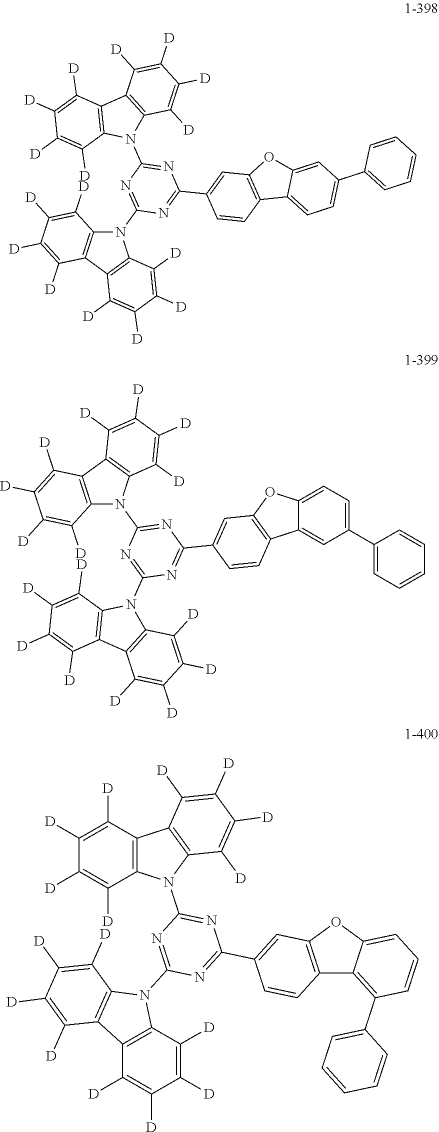

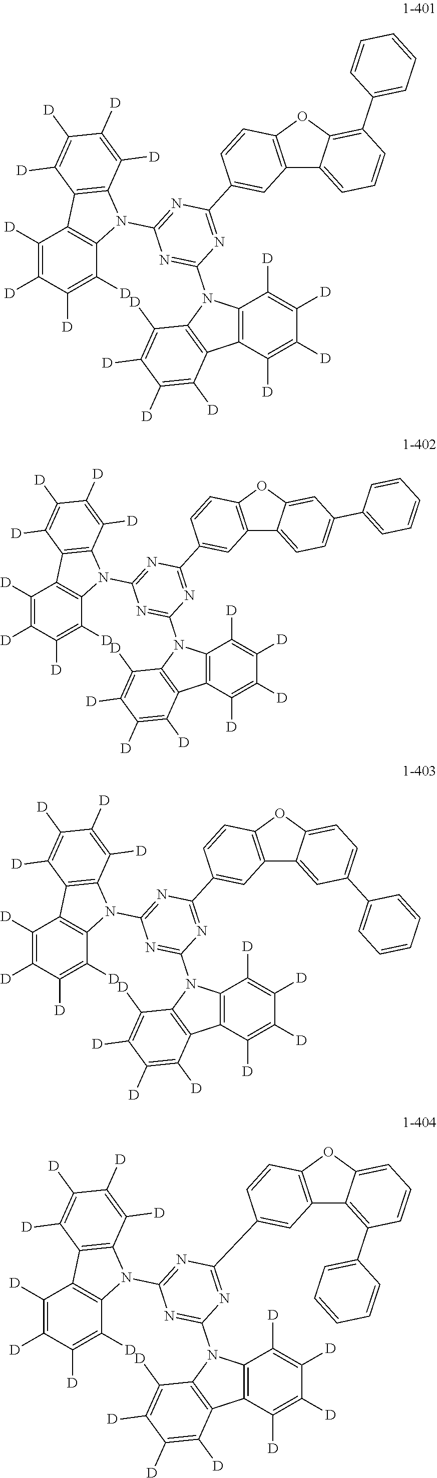

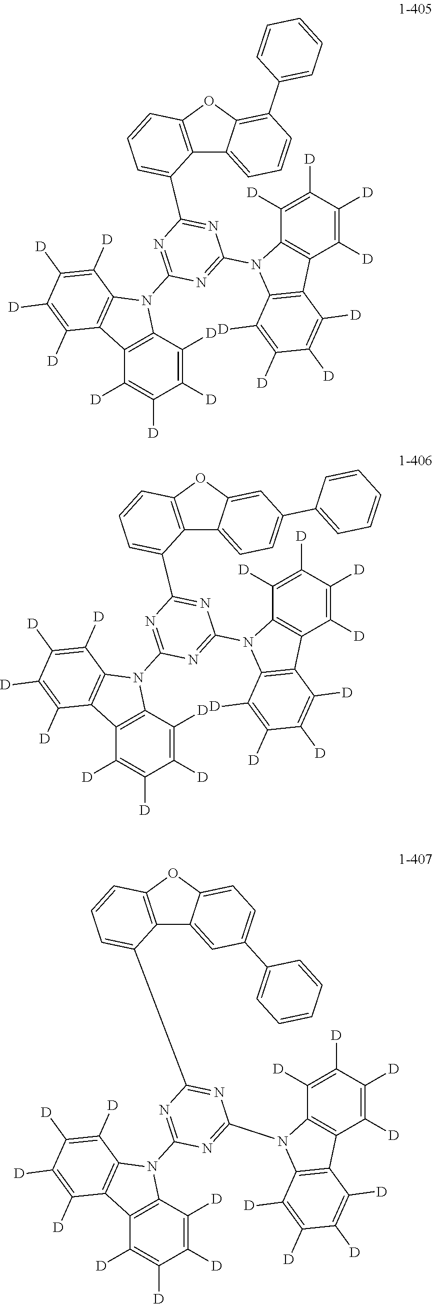

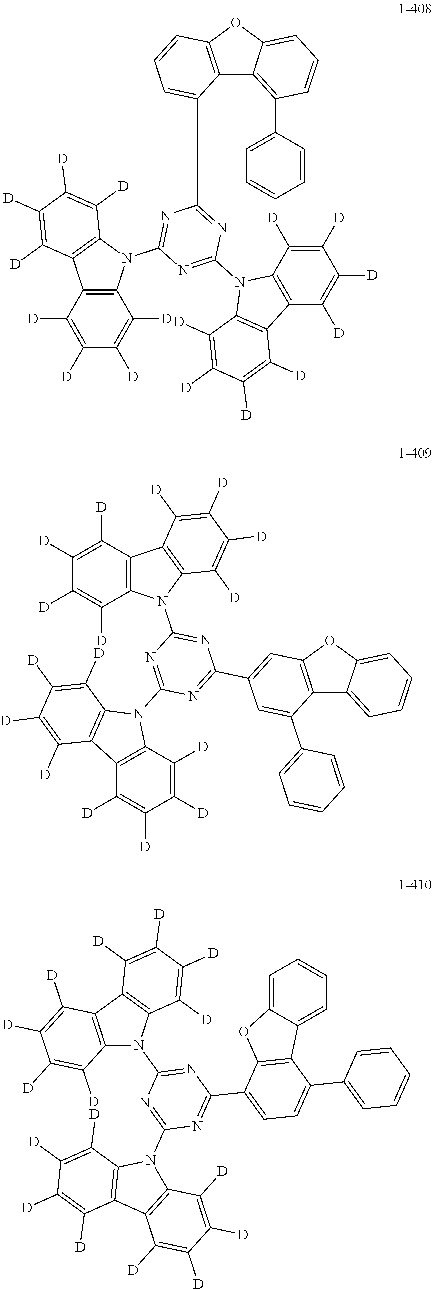

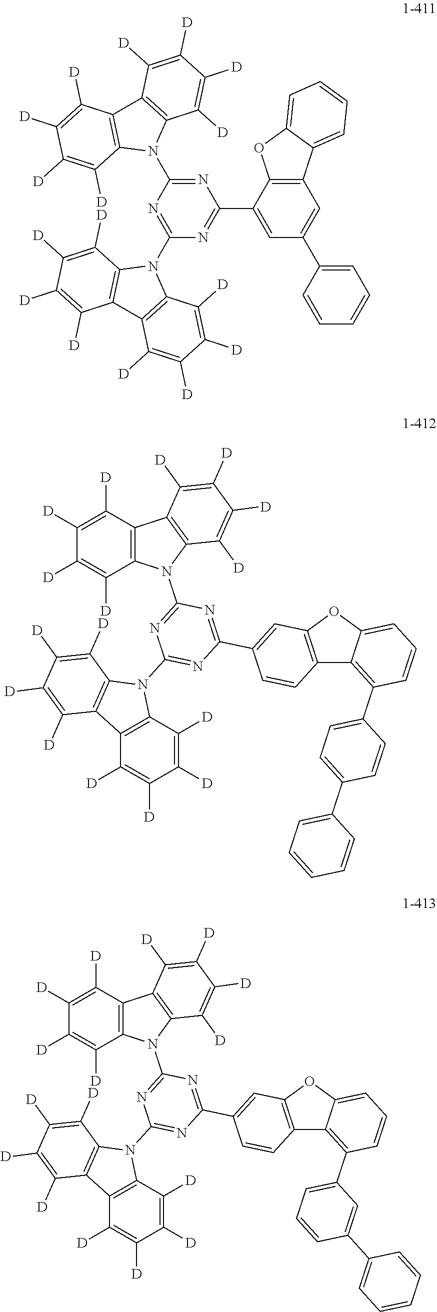

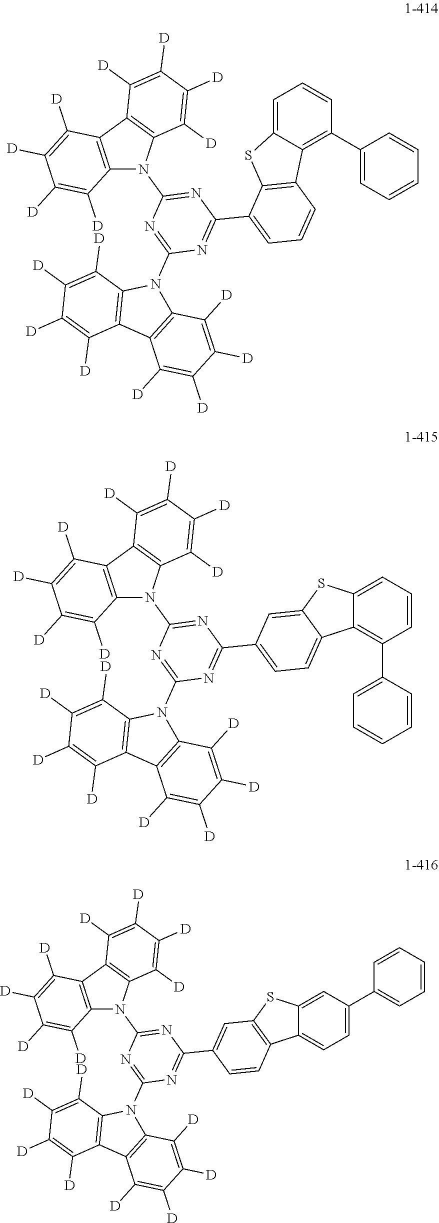

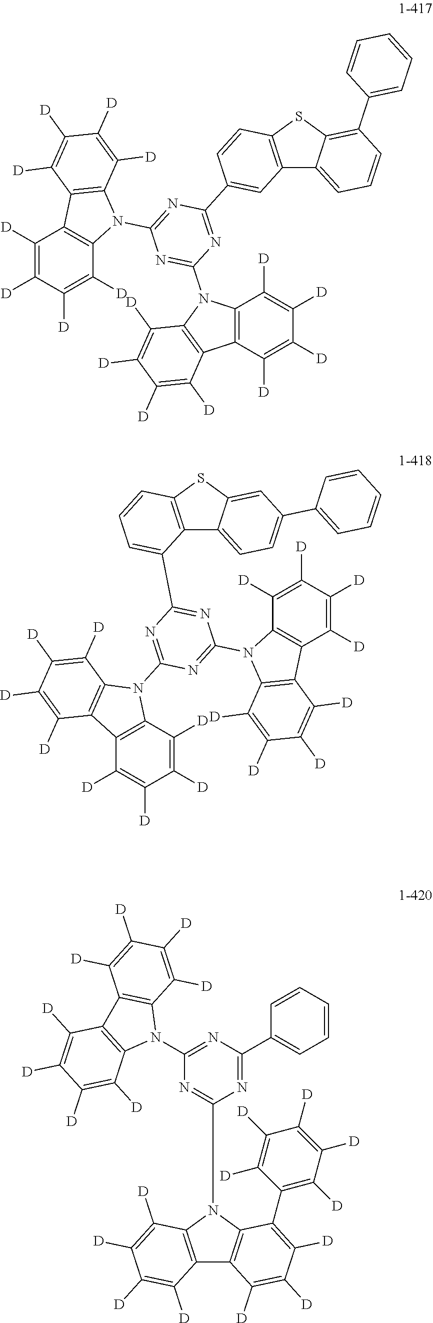

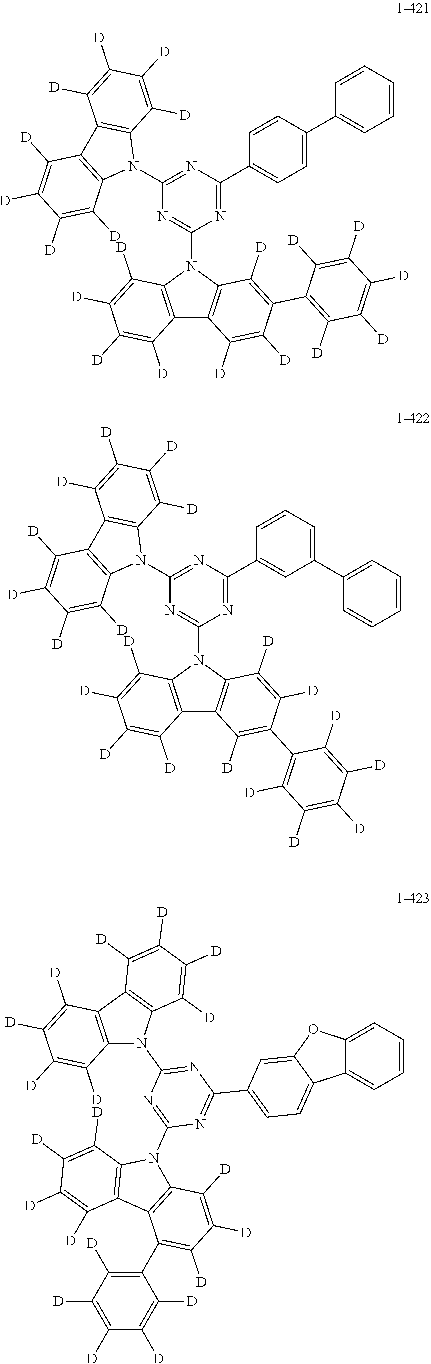

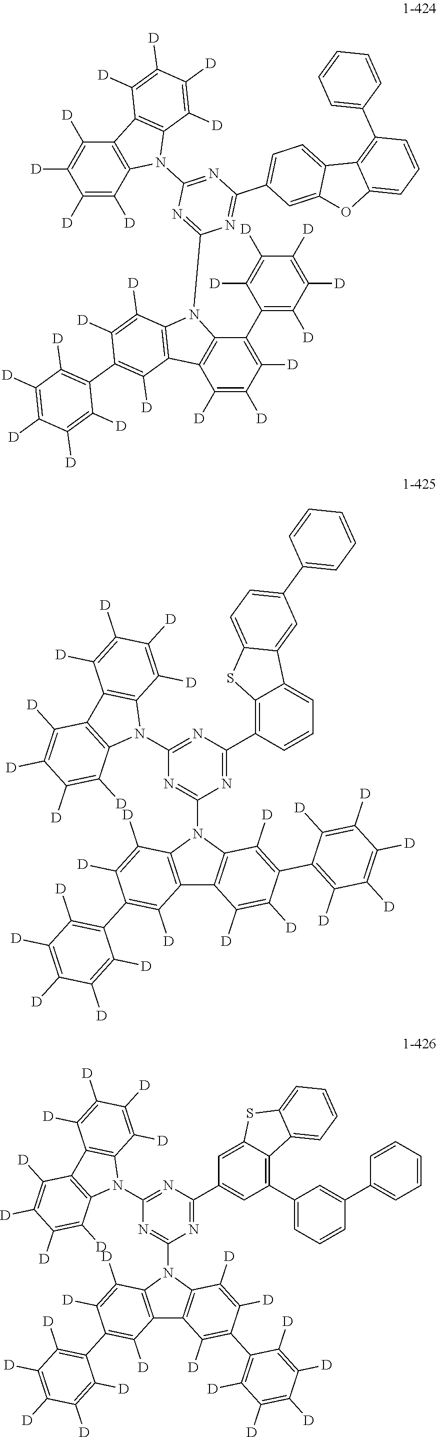

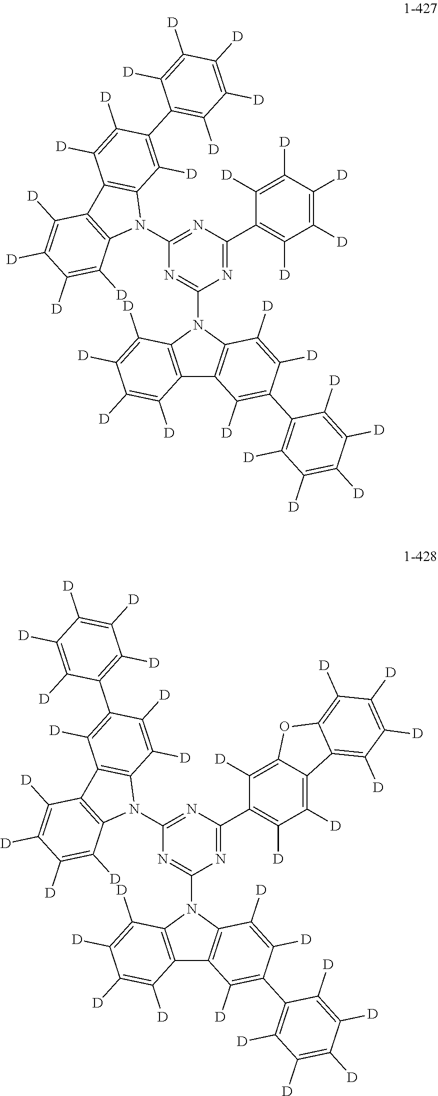

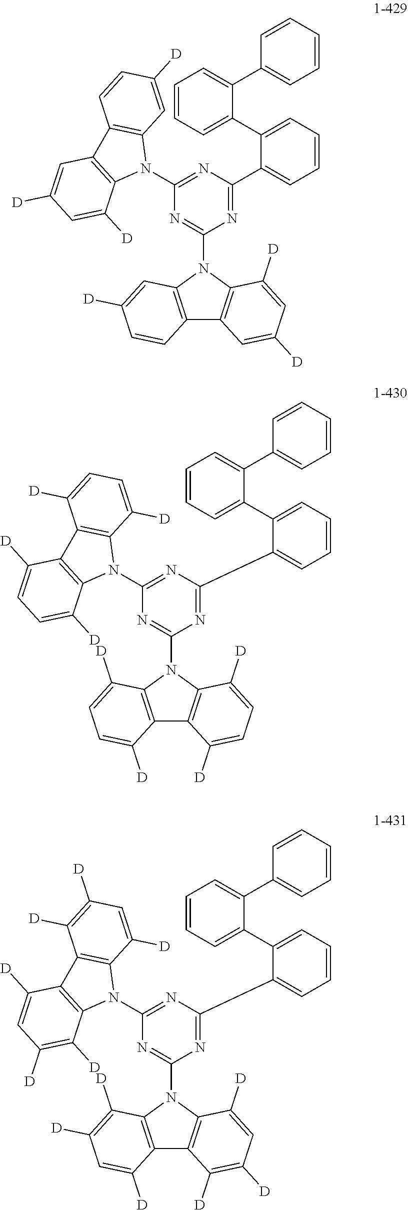

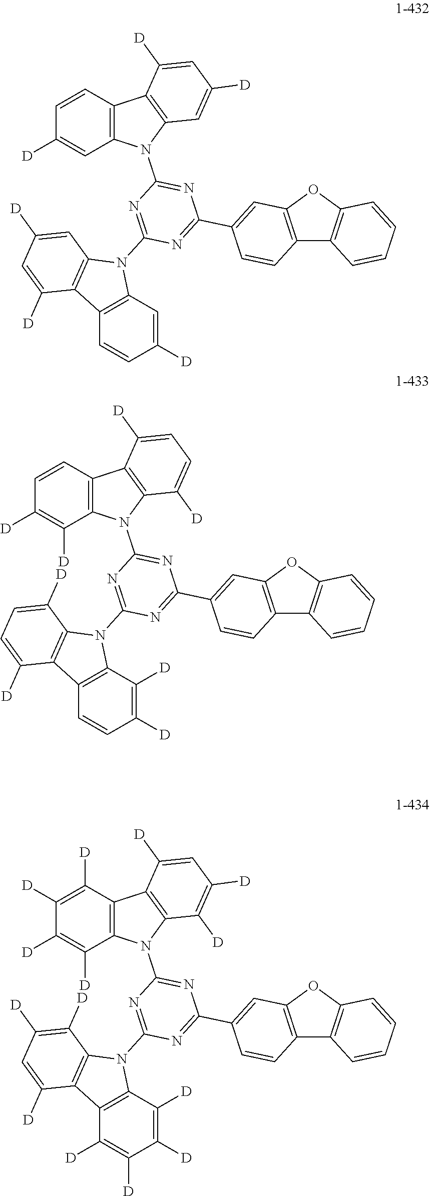 [SMILES: [2H]c1ccc2c3c([2H])cc([2H])cc3n(-c3nc(-c4ccc5c(c4)oc4ccccc45)nc(-n4c5cc([2H])ccc5c5c([2H])cc([2H])cc54)n3)c2c1 ; [2H]c1ccc2c3c([2H])ccc([2H])c3n(-c3nc(-c4ccc5c(c4)oc4ccccc45)nc(-n4c5c([2H])c([2H])ccc5c5c([2H])ccc([2H])c54)n3)c2c1[2H] ; [2H]c1cc([2H])c2c3c([2H])c([2H])c([2H])c([2H])c3n(-c3nc(-c4ccc5c(c4)oc4ccccc45)nc(-n4c5c([2H])c([2H])cc([2H])c5c5c([2H])c([2H])c([2H])c([2H])c54)n3)c2c1[2H]]

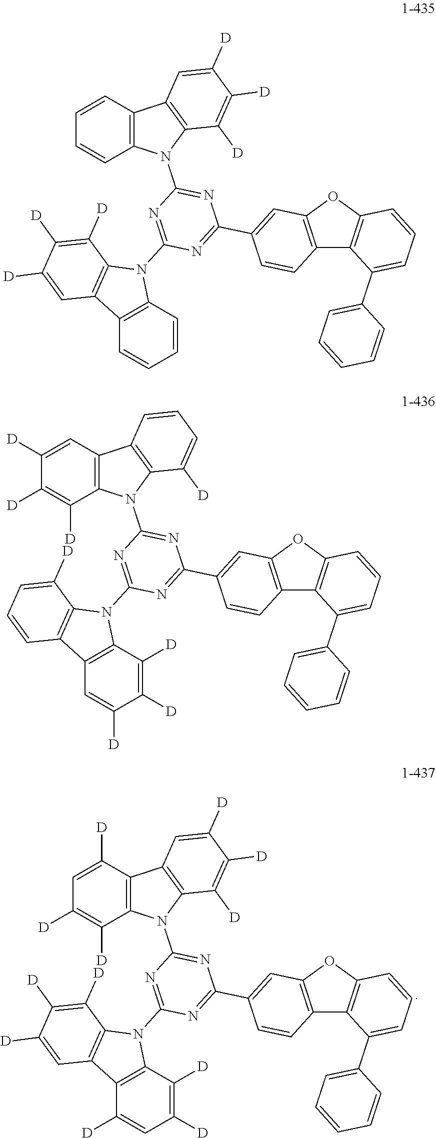

6. An organic light emitting device comprising:
a first electrode;
a second electrode provided to face the first electrode; and
an organic material layer having one or more layers provided between the first electrode and the second electrode,
wherein one or more layers of the organic material layer comprise the heterocyclic compound according to claim 1.

7. The organic light emitting device of claim 6, wherein the organic material layer comprising the heterocyclic compound further comprises a heterocyclic compound represented by the following Chemical Formula 2 or 3: 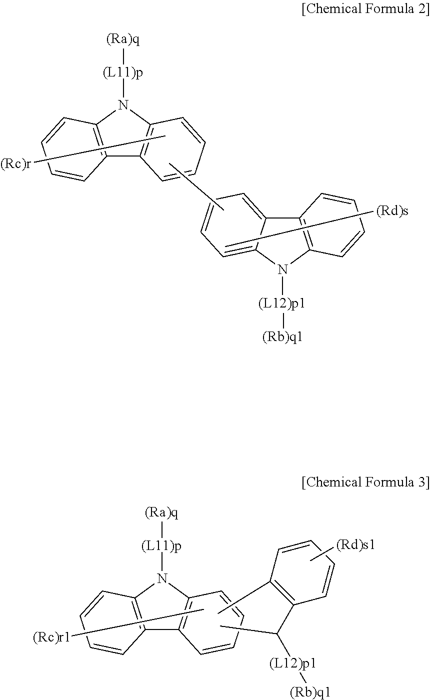
in Chemical Formulae 2 and 3,
Rc and Rd are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group,
L11 and L12 are the same as or different from each other, and are each independently a direct bond; a substituted or unsubstituted C6 to C60 arylene group; or a substituted or unsubstituted C2 to C60 heteroarylene group,
Ra and Rb are the same as or different from each other, and are each independently a substituted or unsubstituted C6 to C60 aryl group; a substituted or unsubstituted C2 to C60 heteroaryl group; a cyano group; or —SiRR′R″,
R, R′, and R″ are the same as or different from each other, and are each independently hydrogen; deuterium; —CN; a substituted or unsubstituted C1 to C60 alkyl group; a substituted or unsubstituted C6 to C60 aryl group; or a substituted or unsubstituted C2 to C60 heteroaryl group,
p, p1, q, and q1 are an integer from 1 to 4,
r and s are an integer from 0 to 7,
r1 is an integer from 0 to 6,
s1 is an integer from 0 to 4, and
when p, p1, q, q1, r, s, r1, and s1 are 2 or higher, substituent in the parenthesis are the same as or different from each other.

8. The organic light emitting device of claim 7, wherein Chemical Formula 3 is represented by any one of the following Chemical Formulae 3-1 to 3-6: 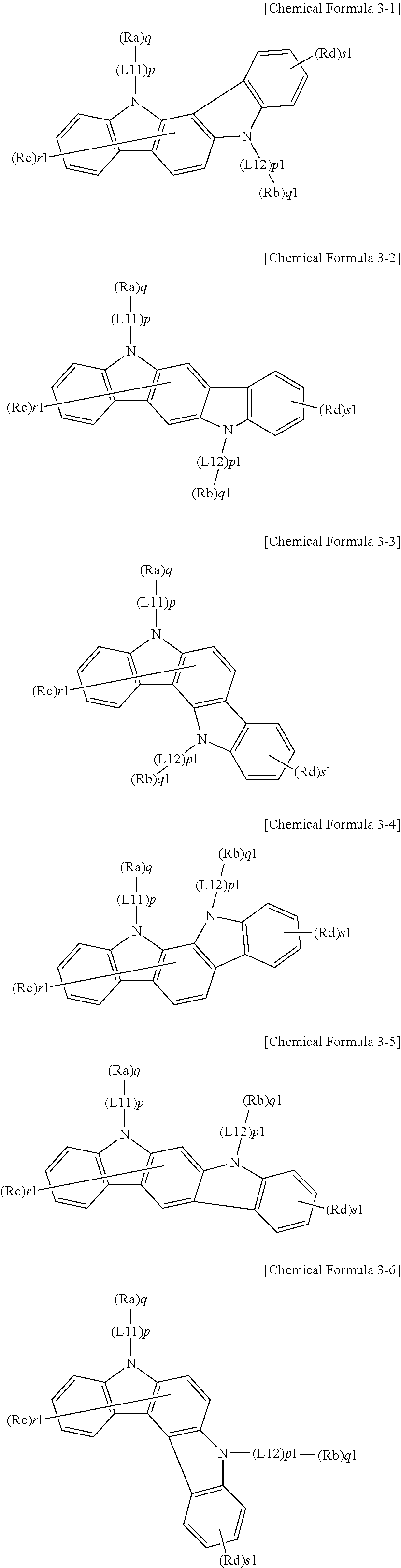
in Chemical Formulae 3-1 to 3-6,
the definition of each substituent is the same as the definition in Chemical Formula 3.

9. The organic light emitting device of claim 7, wherein the heterocyclic compound represented by Chemical Formula 2 or 3 is any one selected from the following compounds:

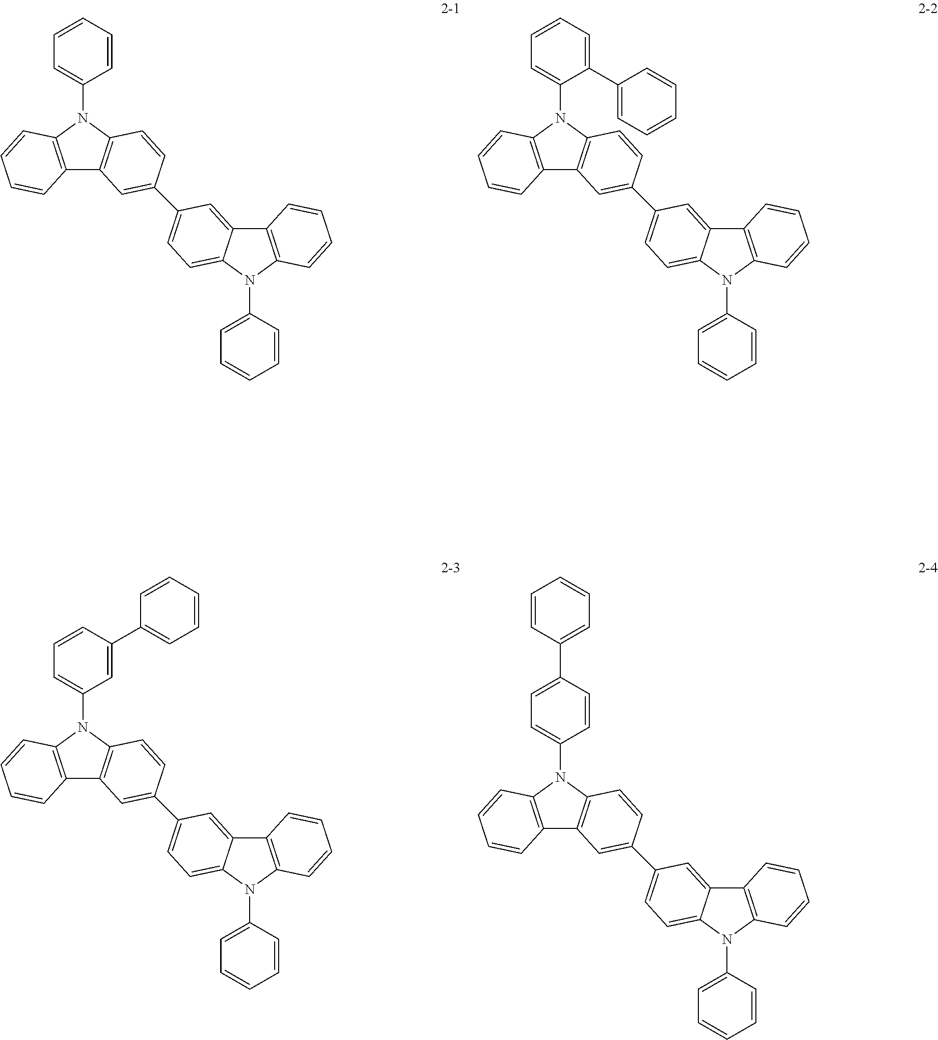 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1]

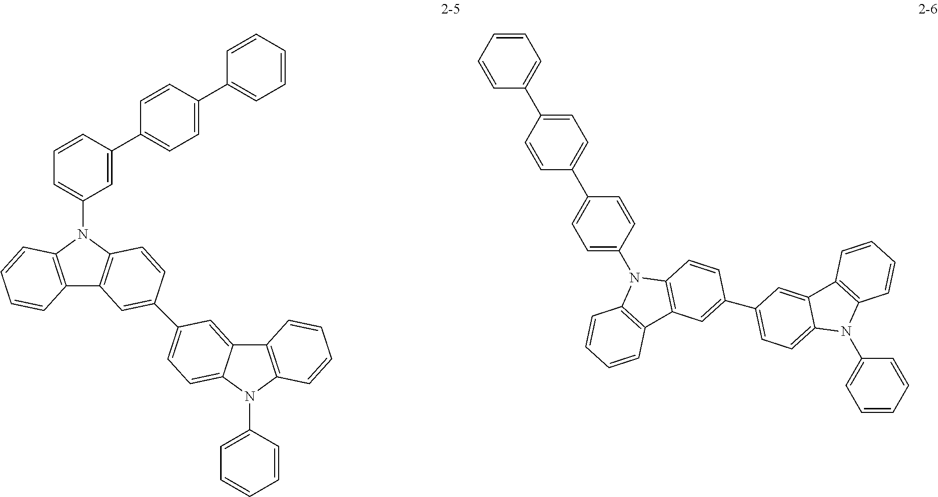 [SMILES: c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

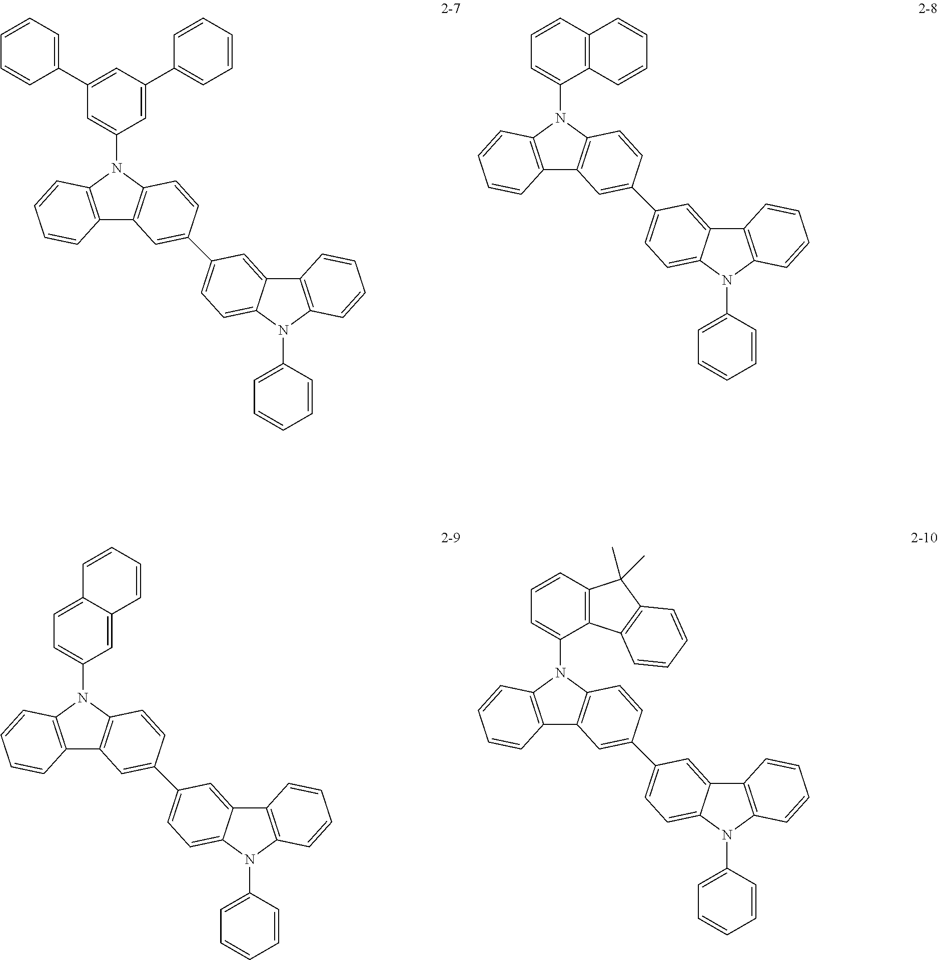 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21]

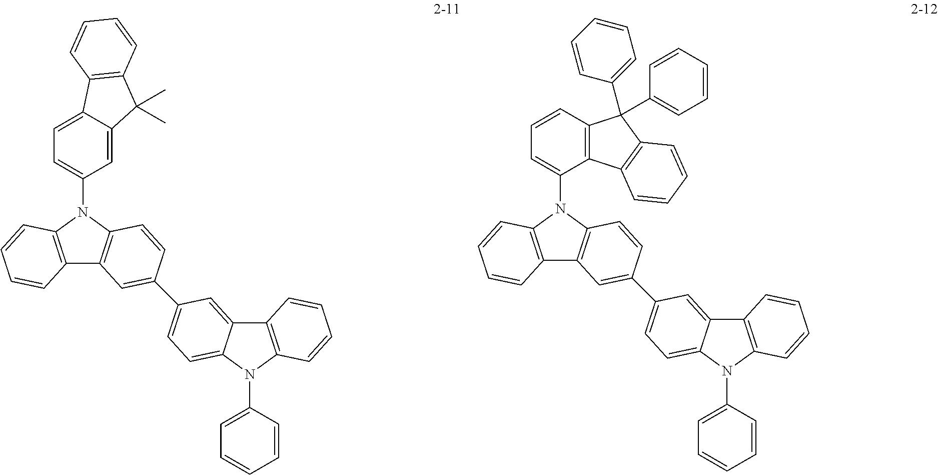 [SMILES: CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

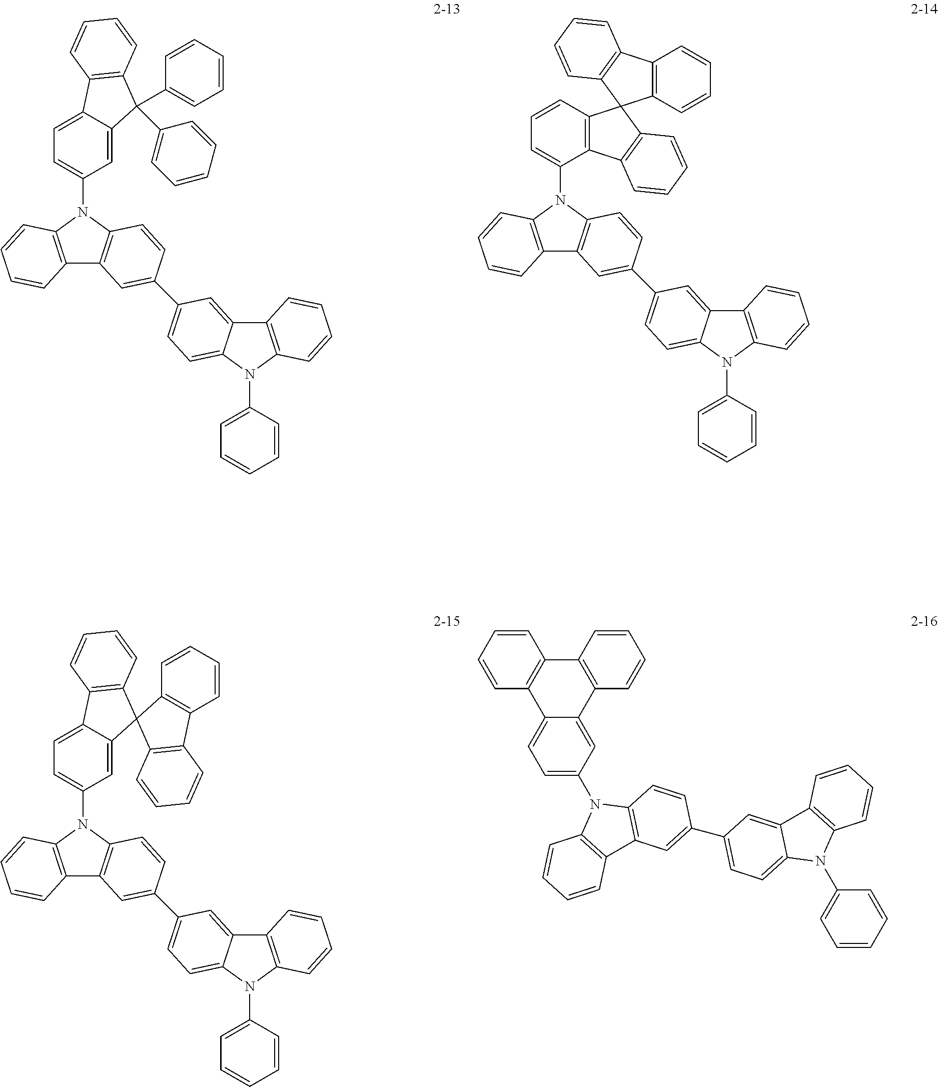 [SMILES: c1ccc(-n2c3ccccc3c3cc(-c4ccc5c(c4)c4ccccc4n5-c4ccc5c(c4)C(c4ccccc4)(c4ccccc4)c4ccccc4-5)ccc32)cc1 ; c1ccc(-n2c3ccccc3c3cc(-c4ccc5c(c4)c4ccccc4n5-c4cccc5c4-c4ccccc4C54c5ccccc5-c5ccccc54)ccc32)cc1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

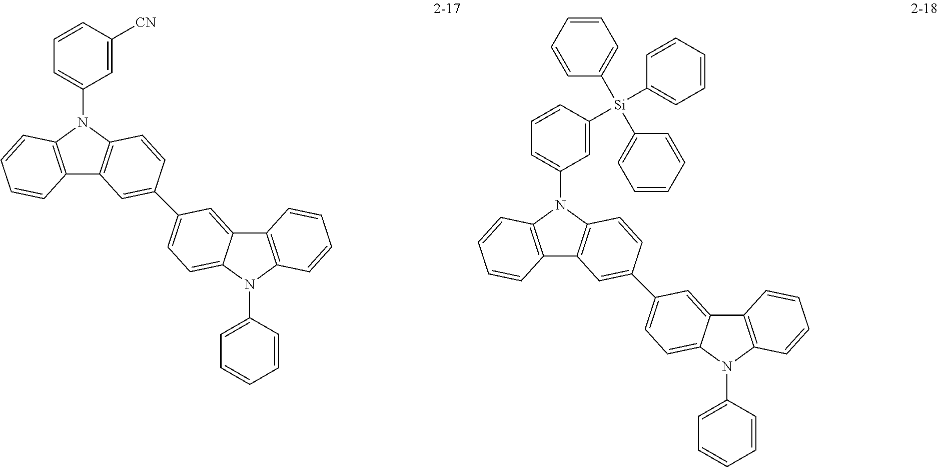 [SMILES: N#Cc1cccc(-n2c3ccccc3c3cc(-c4ccc5c(c4)c4ccccc4n5-c4ccccc4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

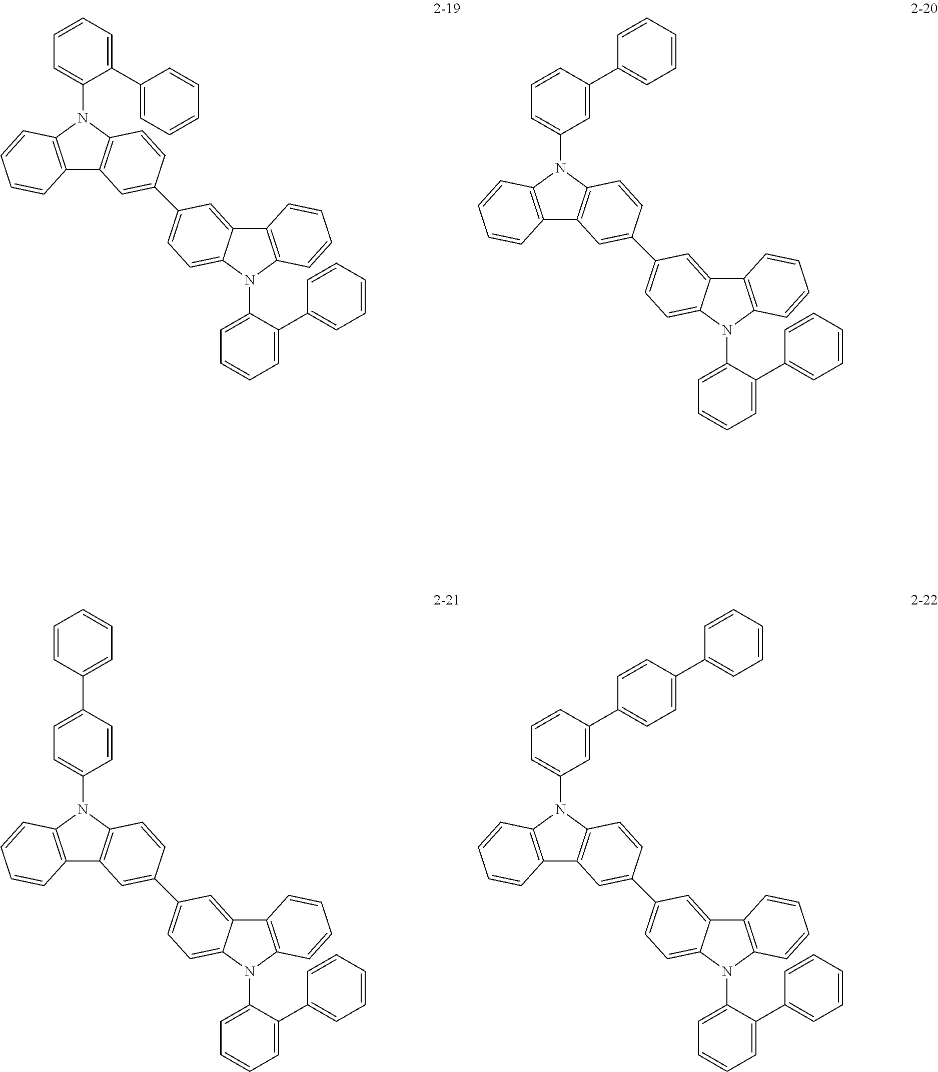 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2ccc(-c3cccc(-n4c5ccccc5c5cc(-c6ccc7c(c6)c6ccccc6n7-c6ccccc6-c6ccccc6)ccc54)c3)cc2)cc1]

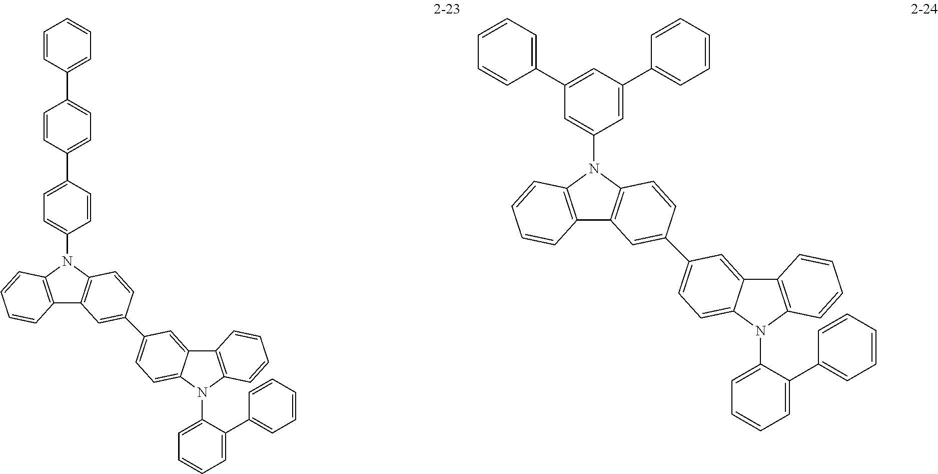 [SMILES: c1ccc(-c2ccc(-c3ccc(-n4c5ccccc5c5cc(-c6ccc7c(c6)c6ccccc6n7-c6ccccc6-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5-c5ccccc5)ccc43)c2)cc1]

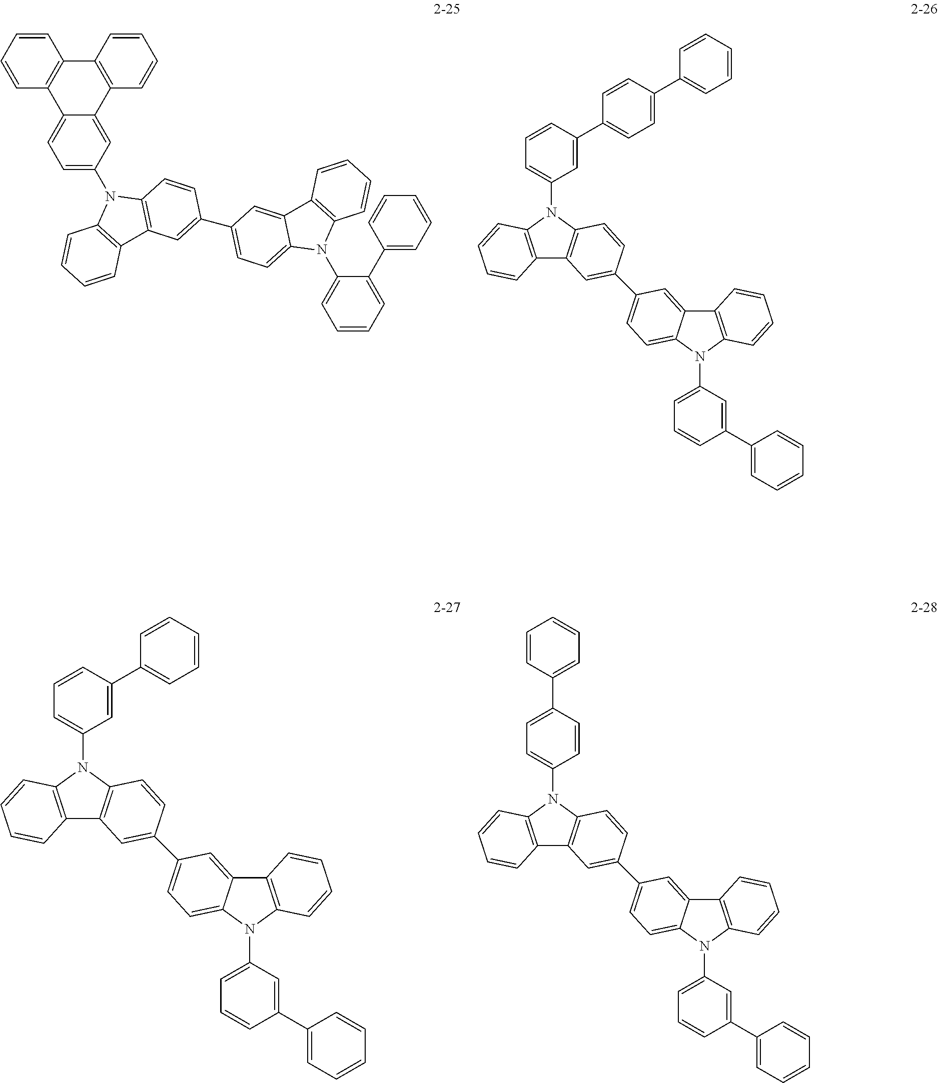 [SMILES: c1ccc(-c2ccccc2-n2c3ccccc3c3cc(-c4ccc5c(c4)c4ccccc4n5-c4ccc5c6ccccc6c6ccccc6c5c4)ccc32)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

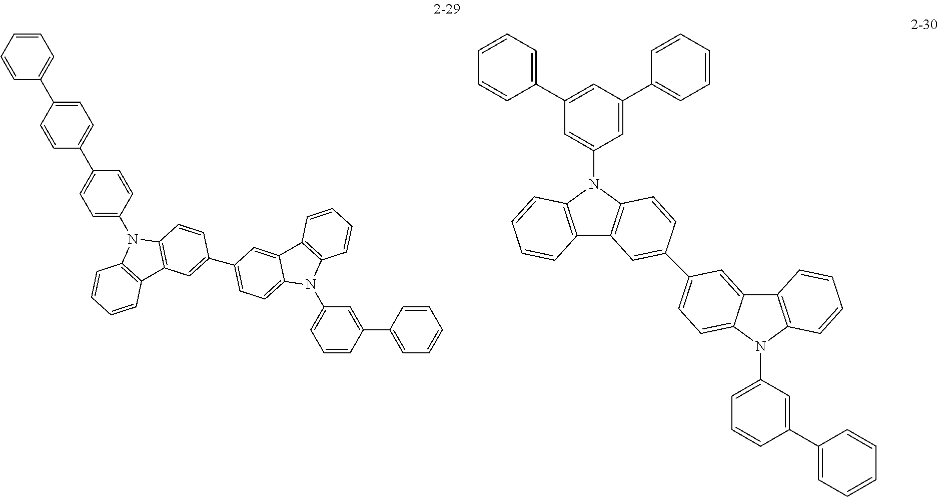 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

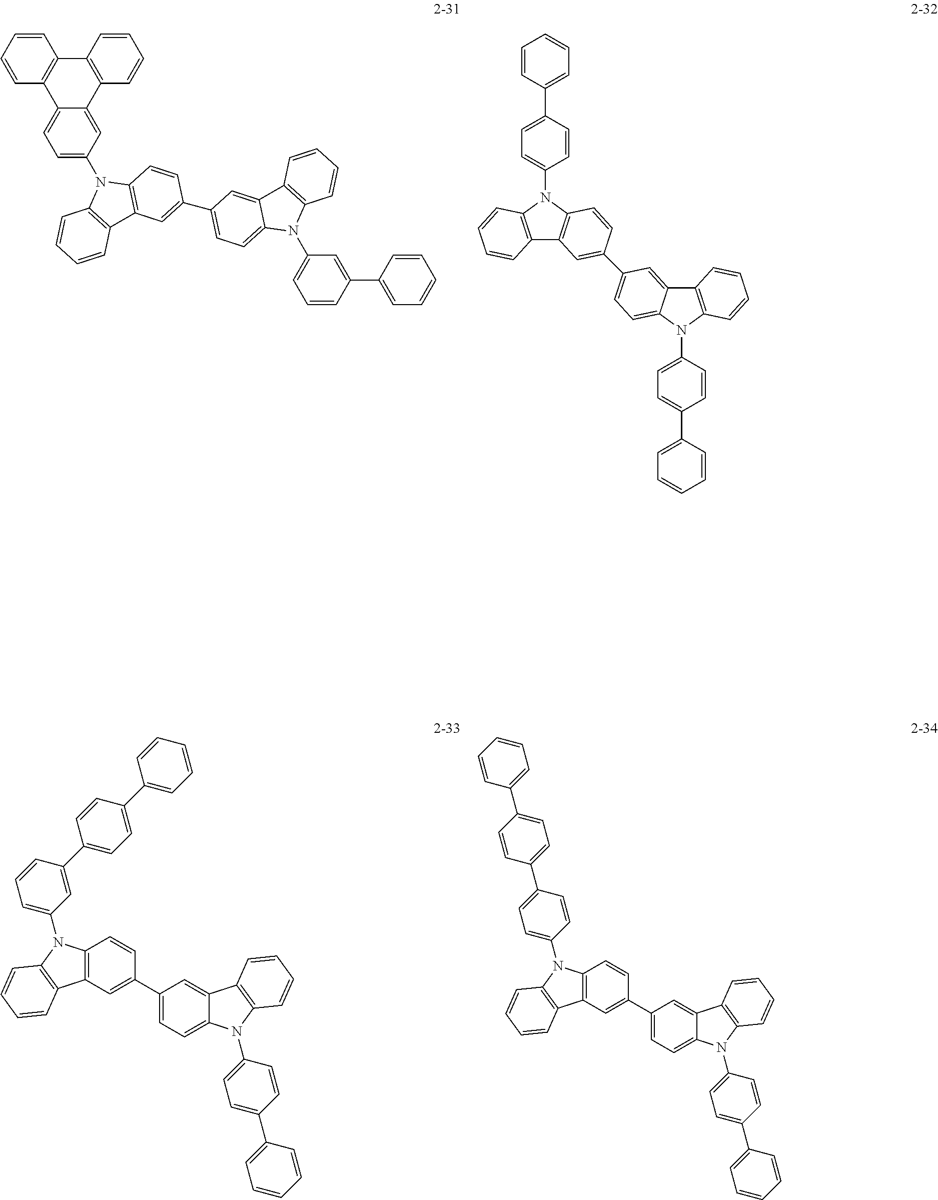 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1]

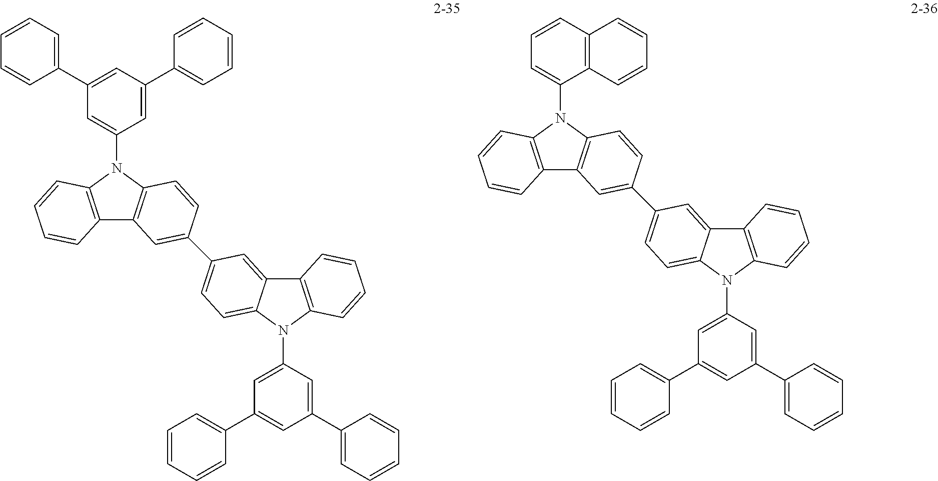 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1]

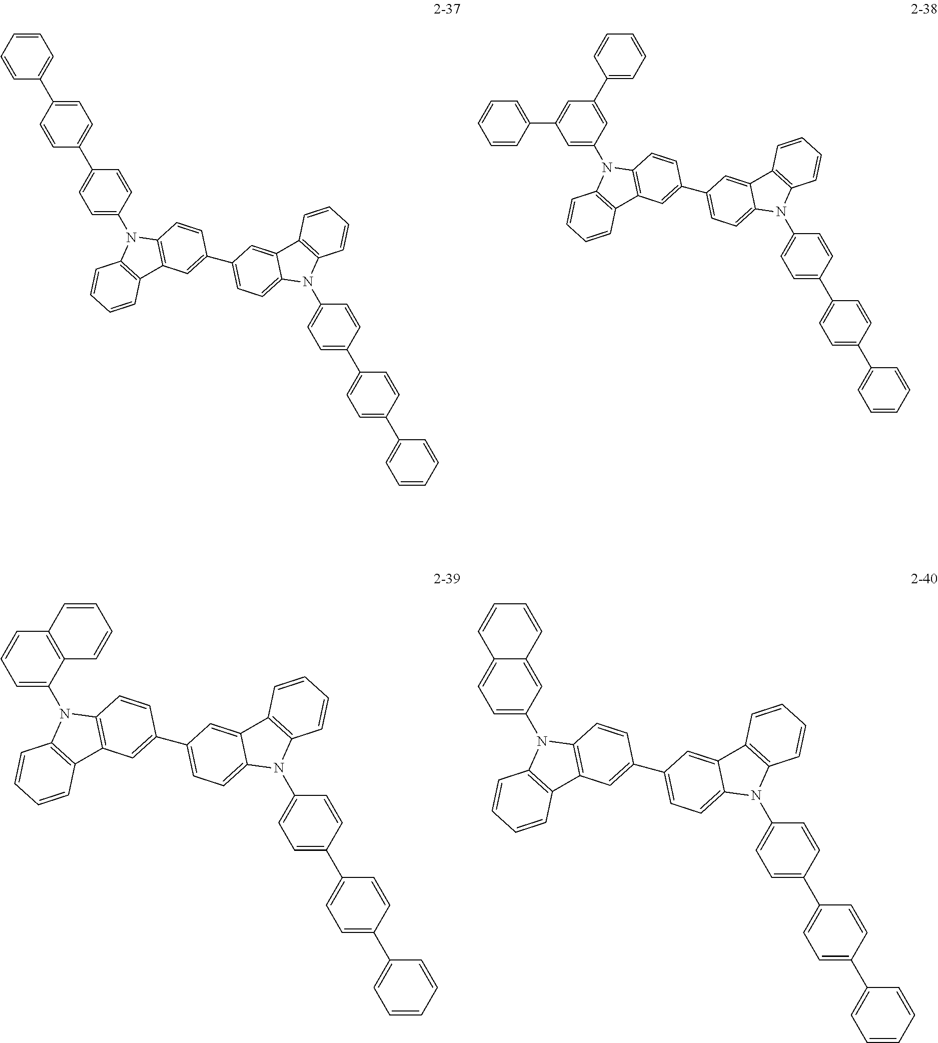 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

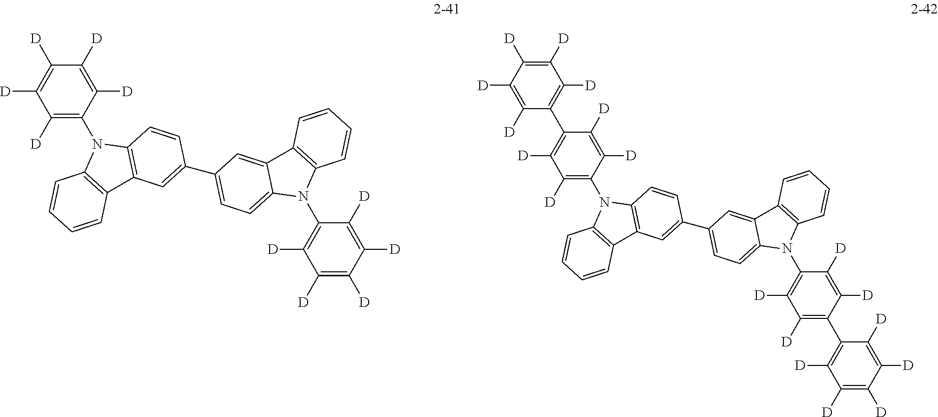 [SMILES: [2H]c1c([2H])c([2H])c(-n2c3ccccc3c3cc(-c4ccc5c(c4)c4ccccc4n5-c4c([2H])c([2H])c([2H])c([2H])c4[2H])ccc32)c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c([2H])c6[2H])c([2H])c5[2H])ccc43)c([2H])c2[2H])c([2H])c1[2H]]

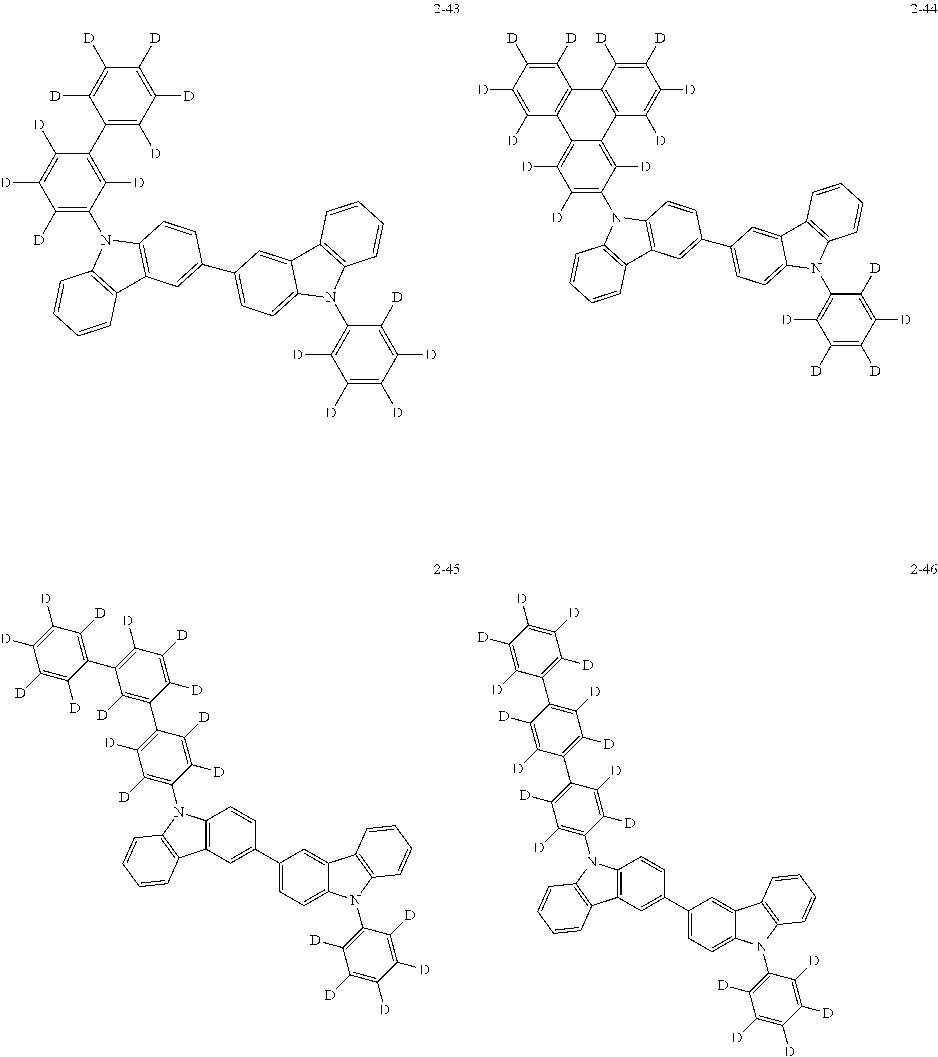

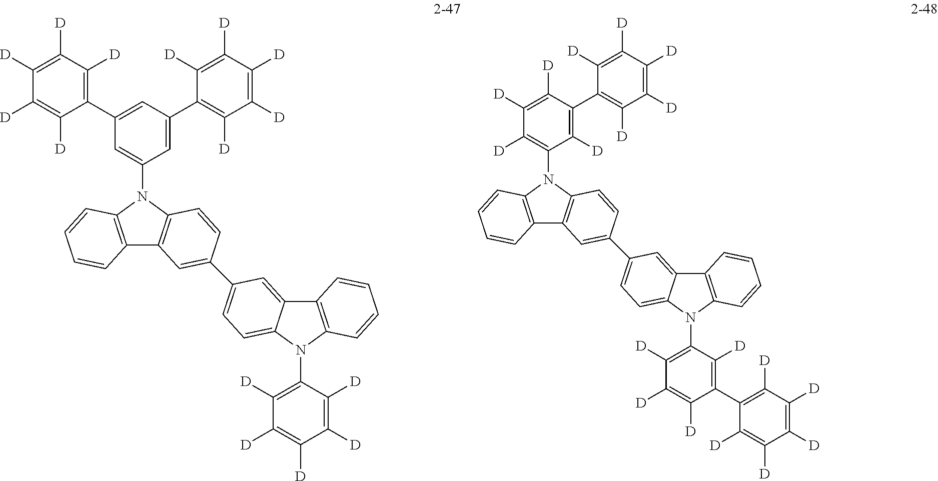 [SMILES: [2H]c1c([2H])c([2H])c(-c2cc(-c3c([2H])c([2H])c([2H])c([2H])c3[2H])cc(-n3c4ccccc4c4cc(-c5ccc6c(c5)c5ccccc5n6-c5c([2H])c([2H])c([2H])c([2H])c5[2H])ccc43)c2)c([2H])c1[2H] ; [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c2[2H])c([2H])c1[2H]]

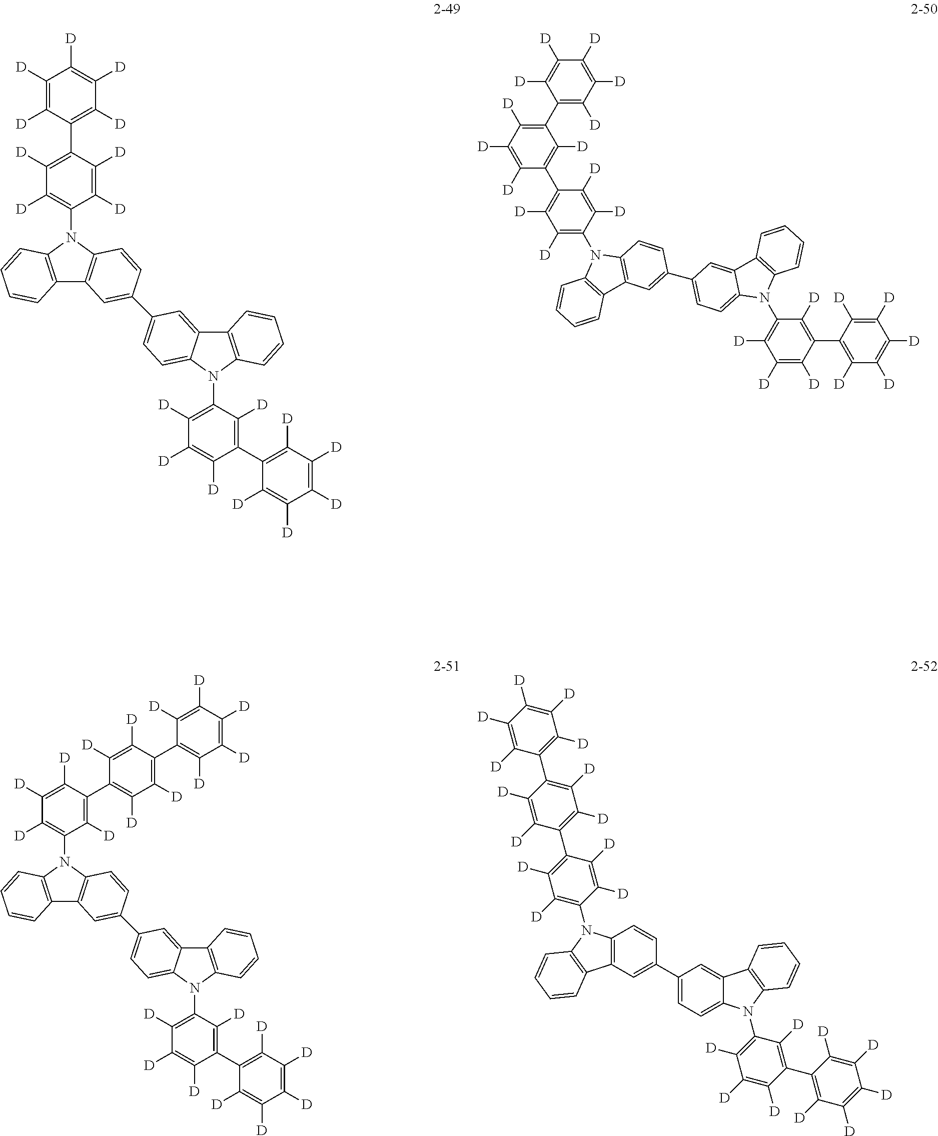

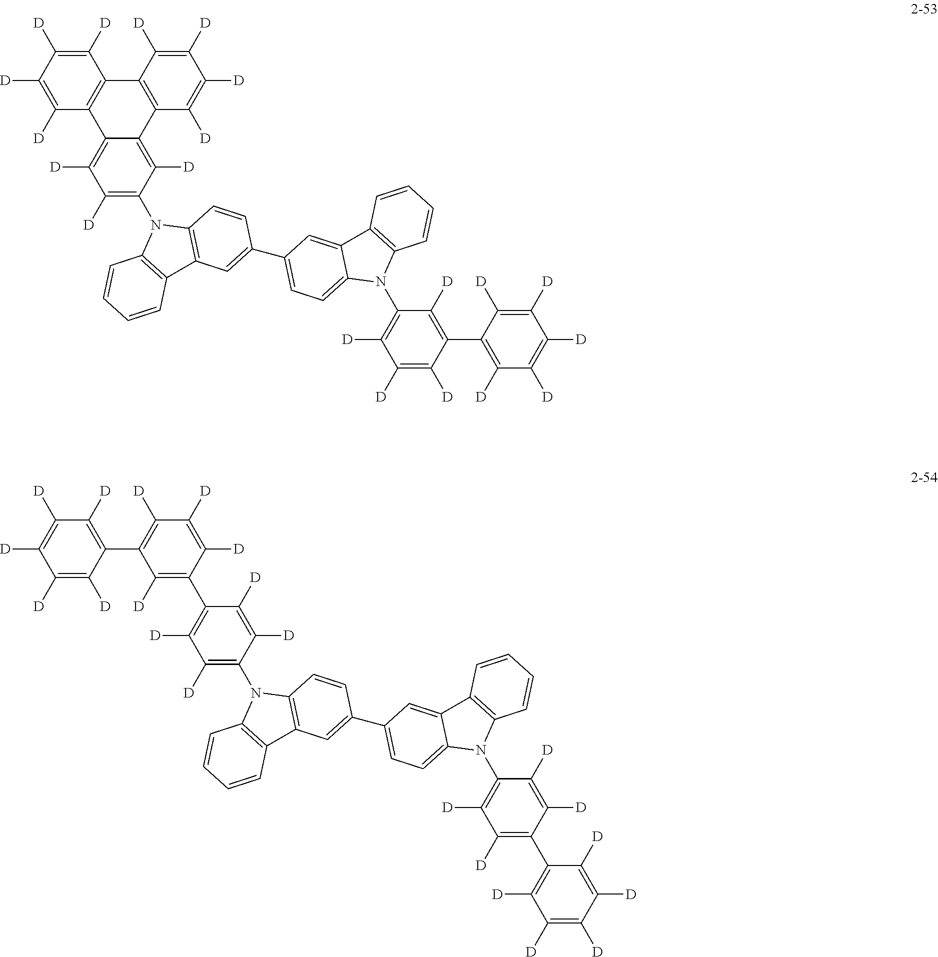 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c6c7c([2H])c([2H])c([2H])c([2H])c7c7c([2H])c([2H])c([2H])c([2H])c7c6c5[2H])ccc43)c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c(-c7c([2H])c([2H])c([2H])c([2H])c7[2H])c6[2H])c([2H])c5[2H])ccc43)c([2H])c2[2H])c([2H])c1[2H]]

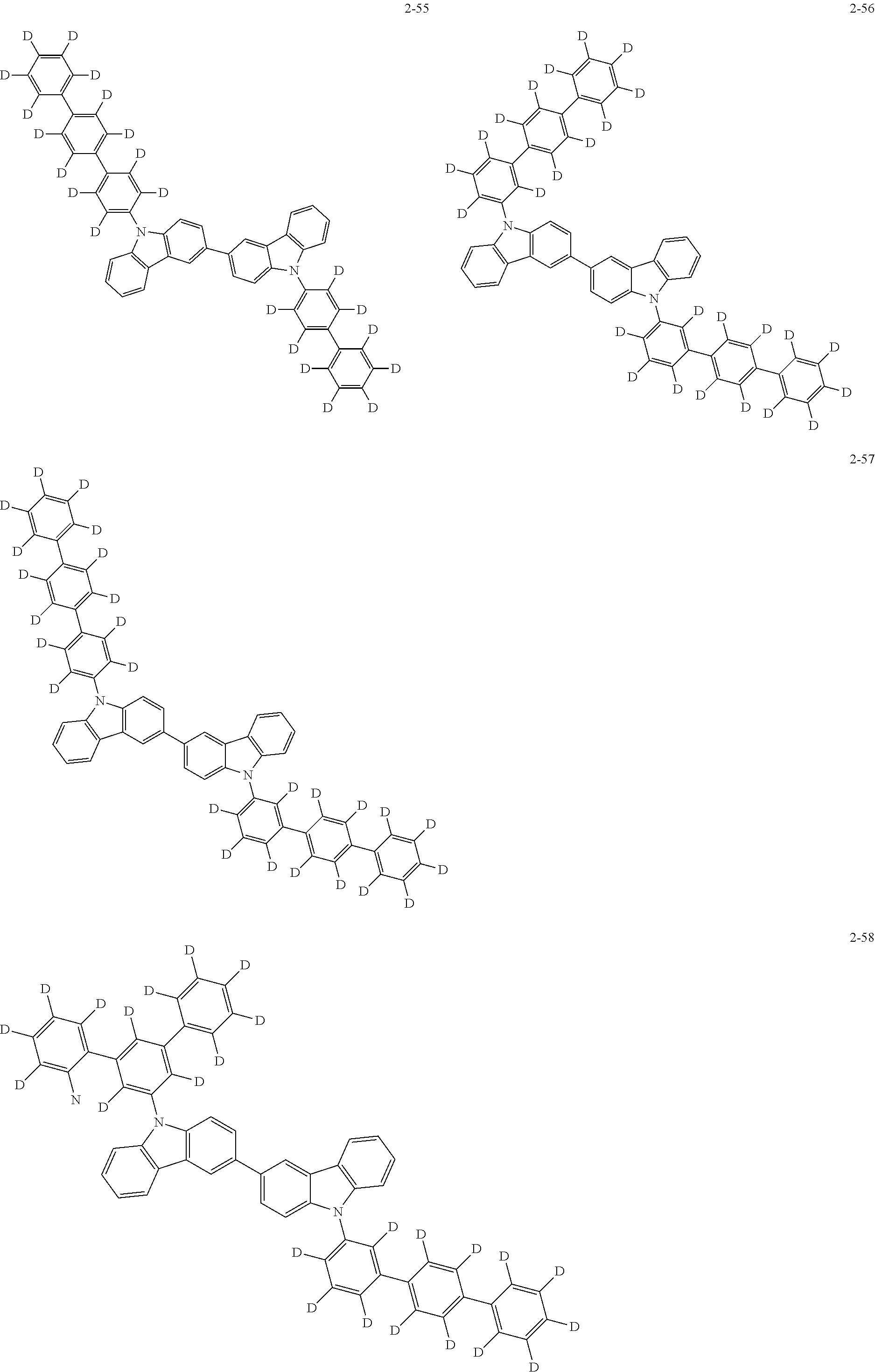

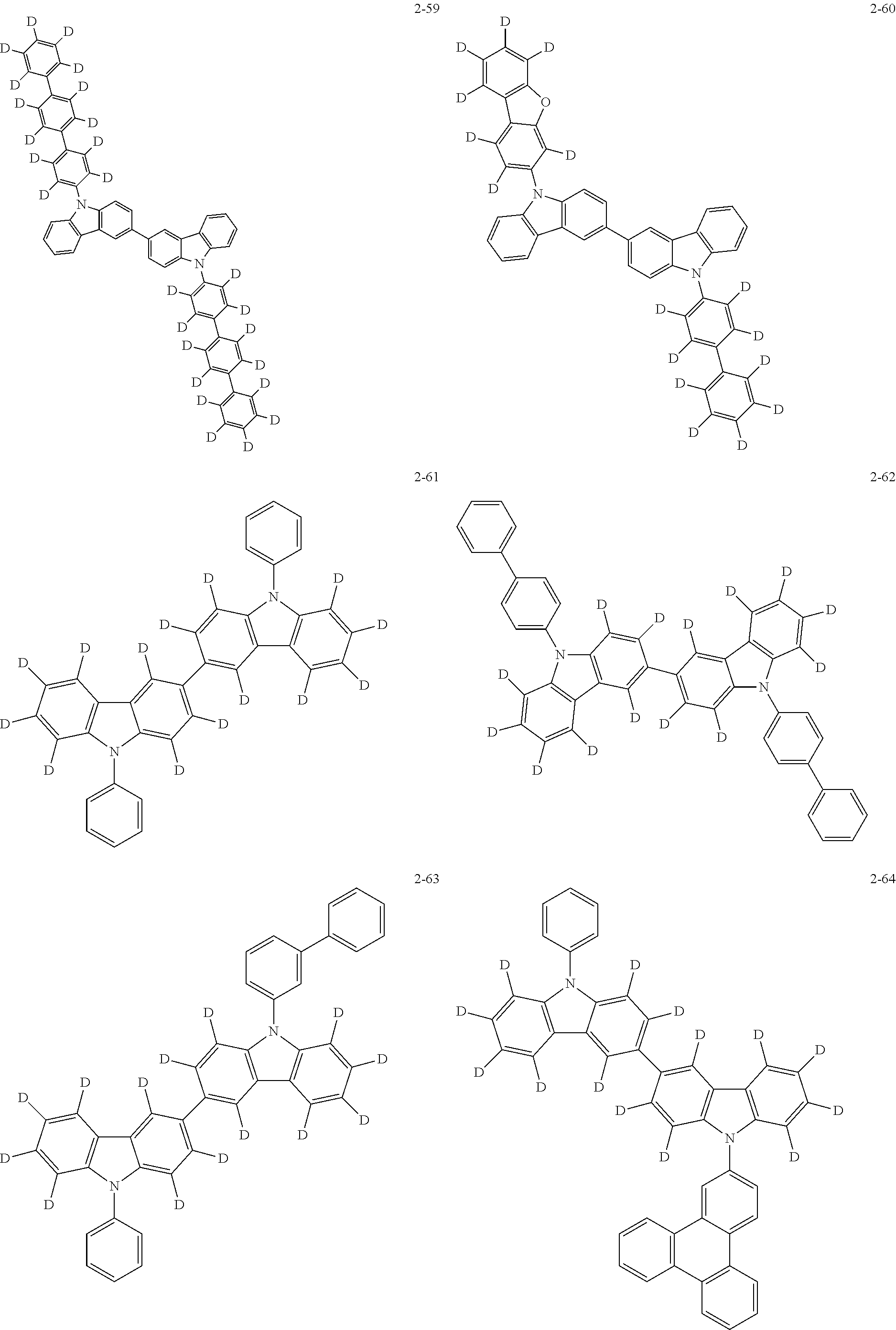

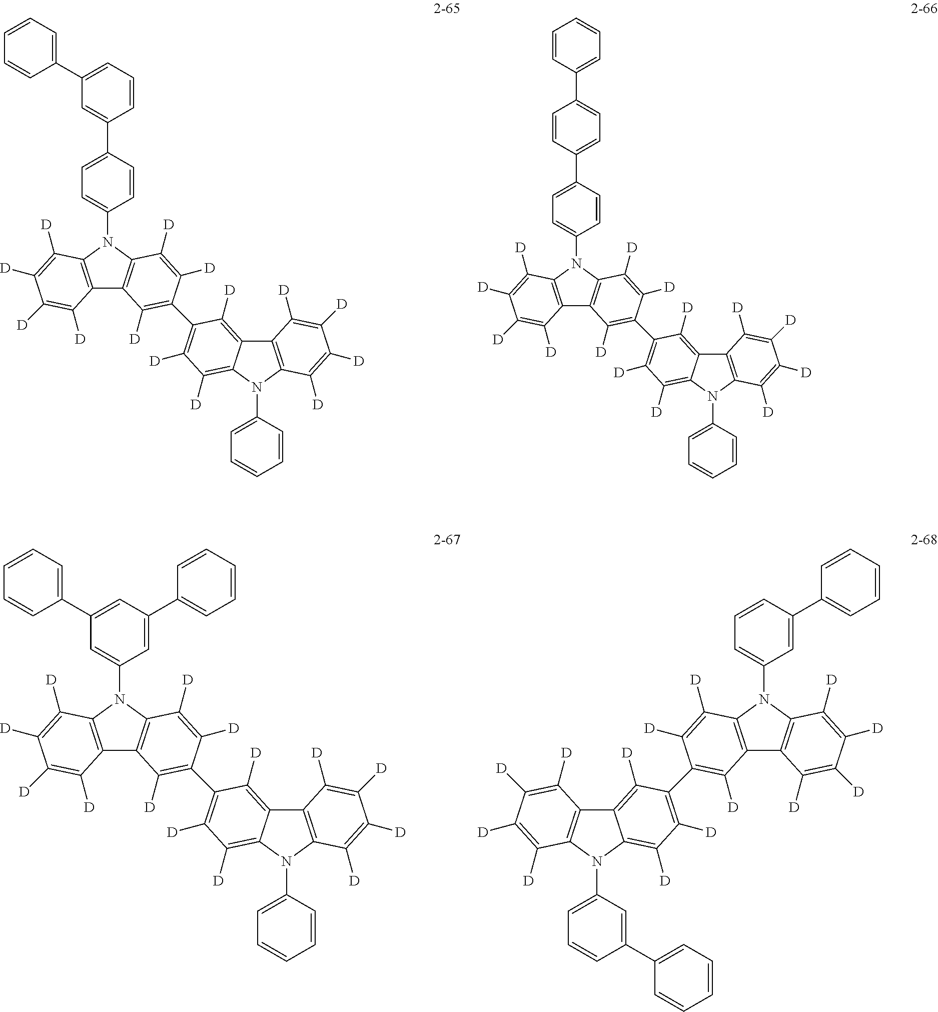

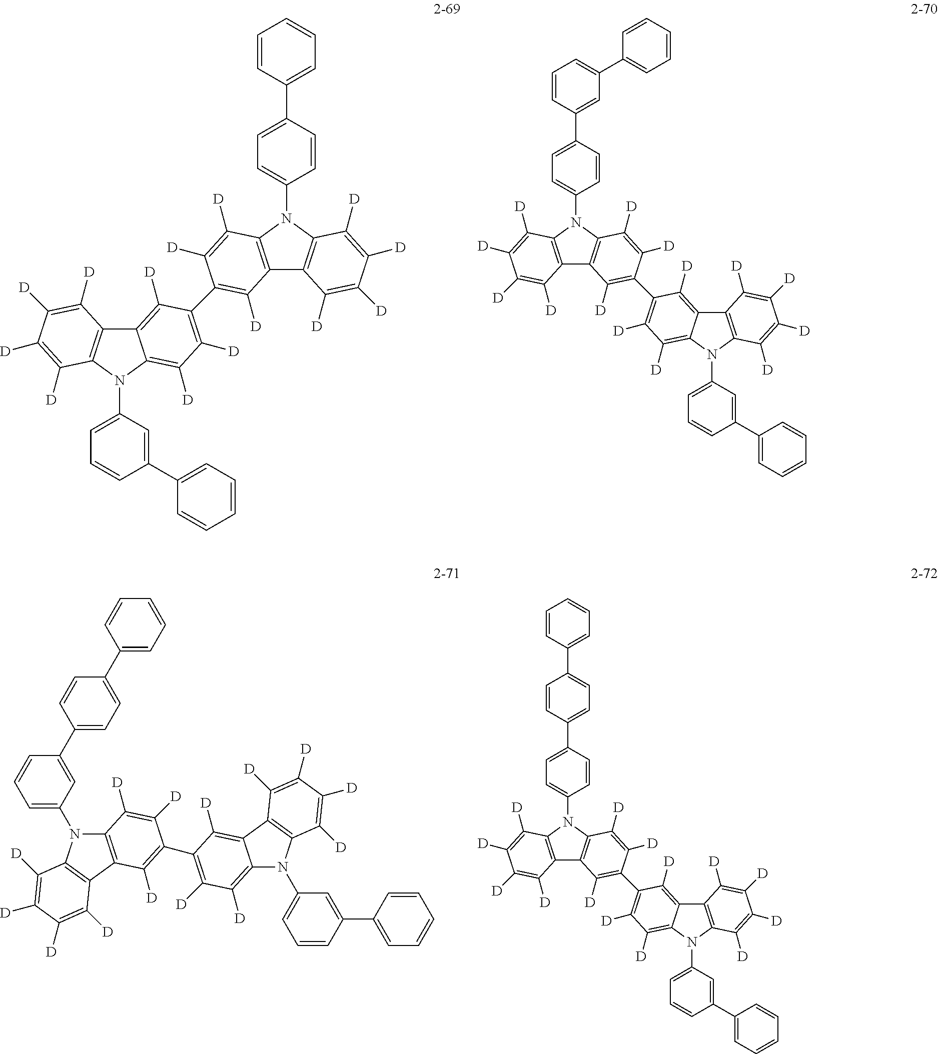

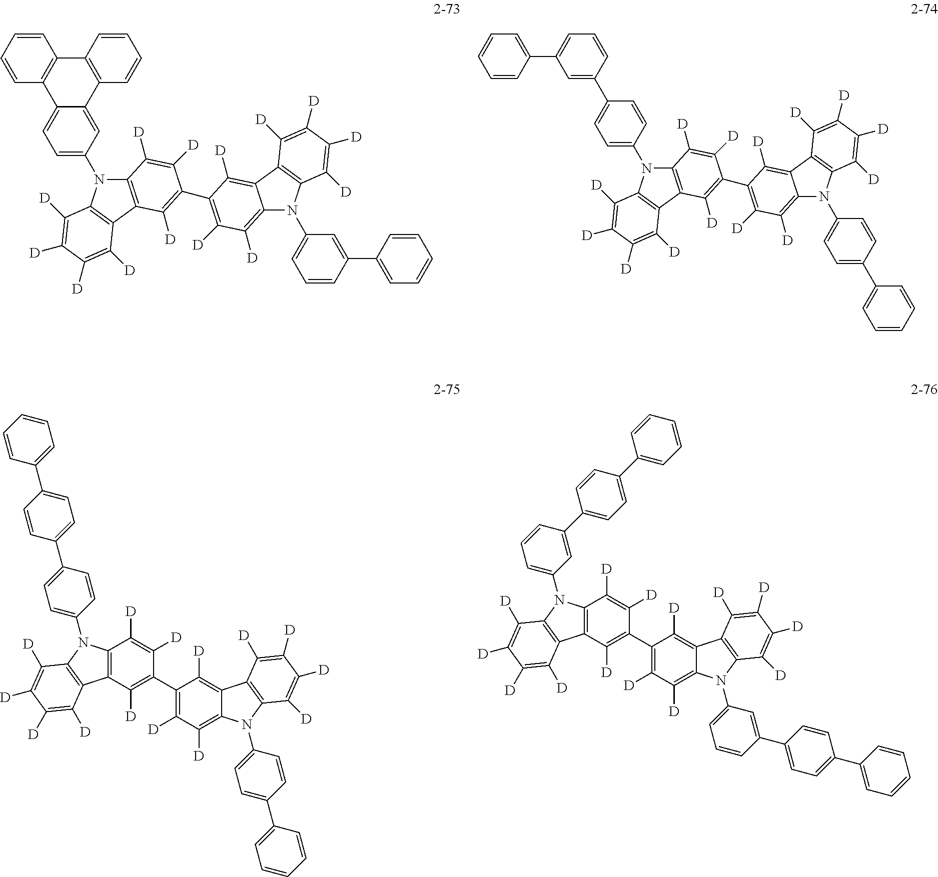

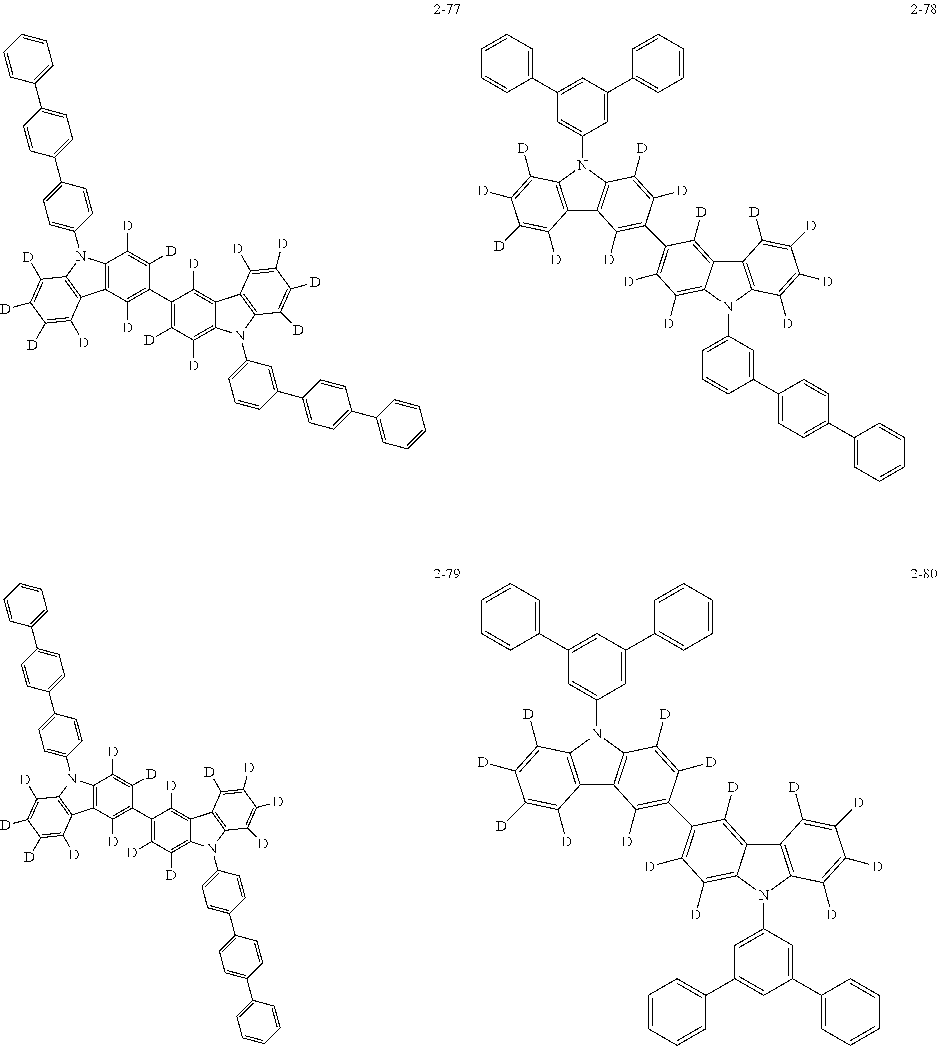

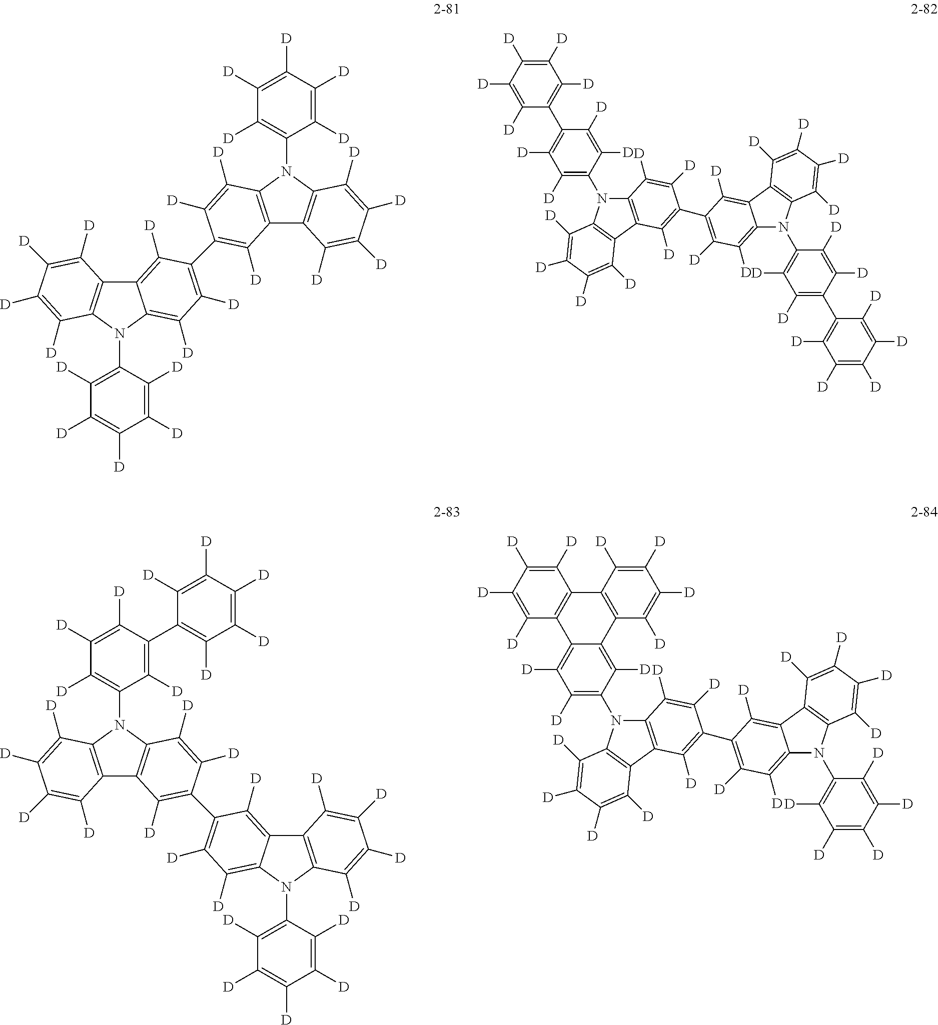

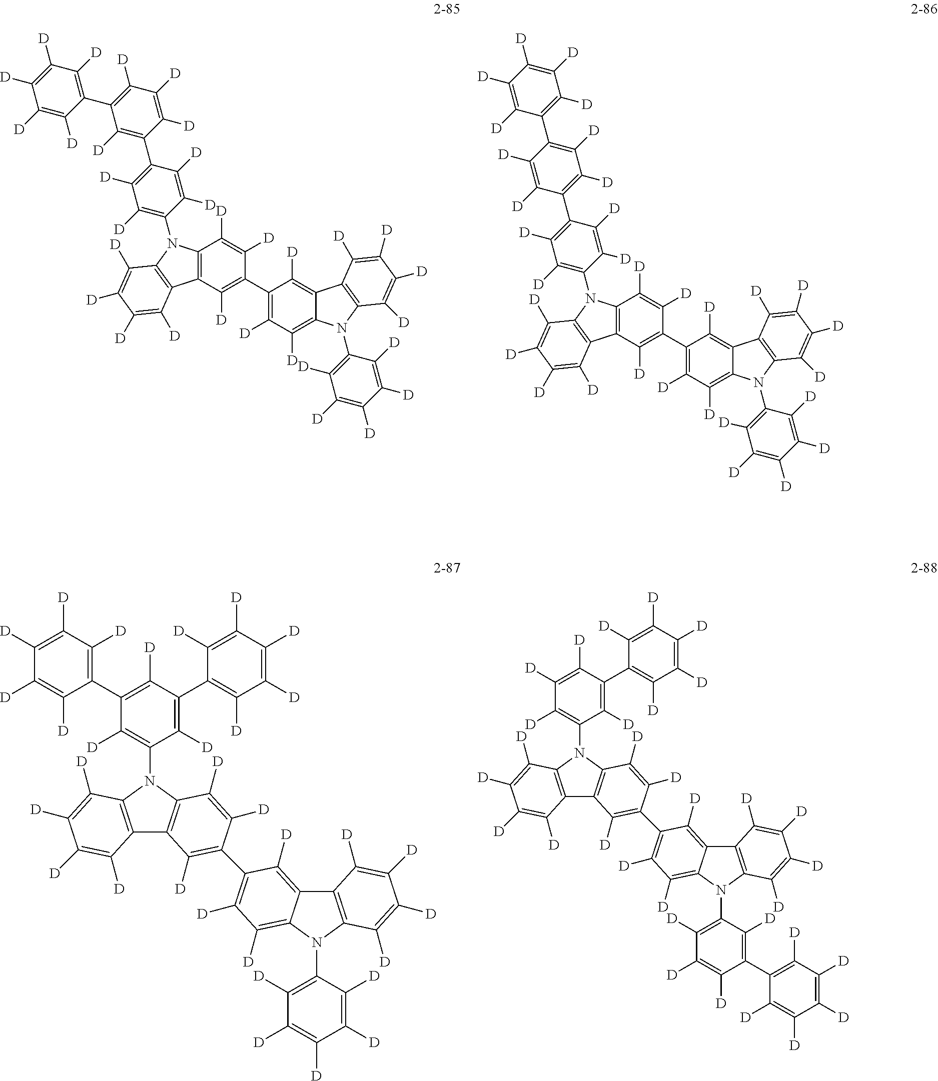

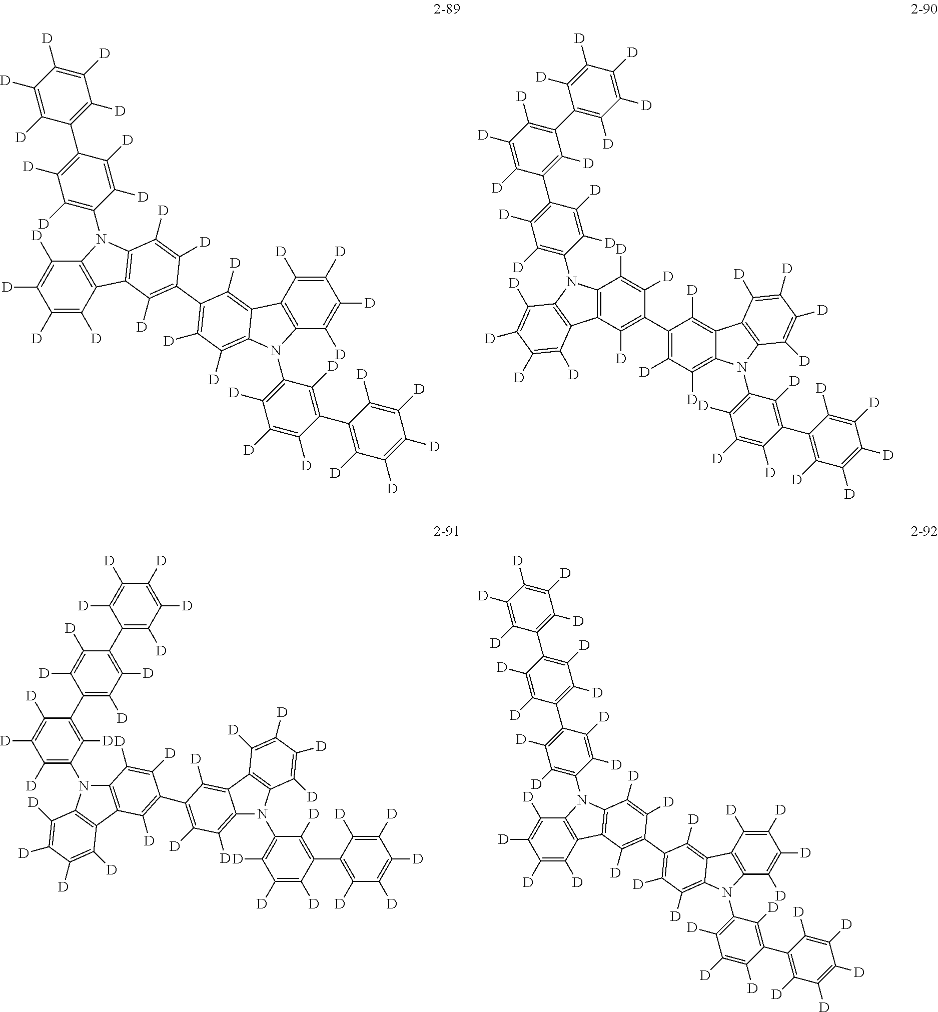

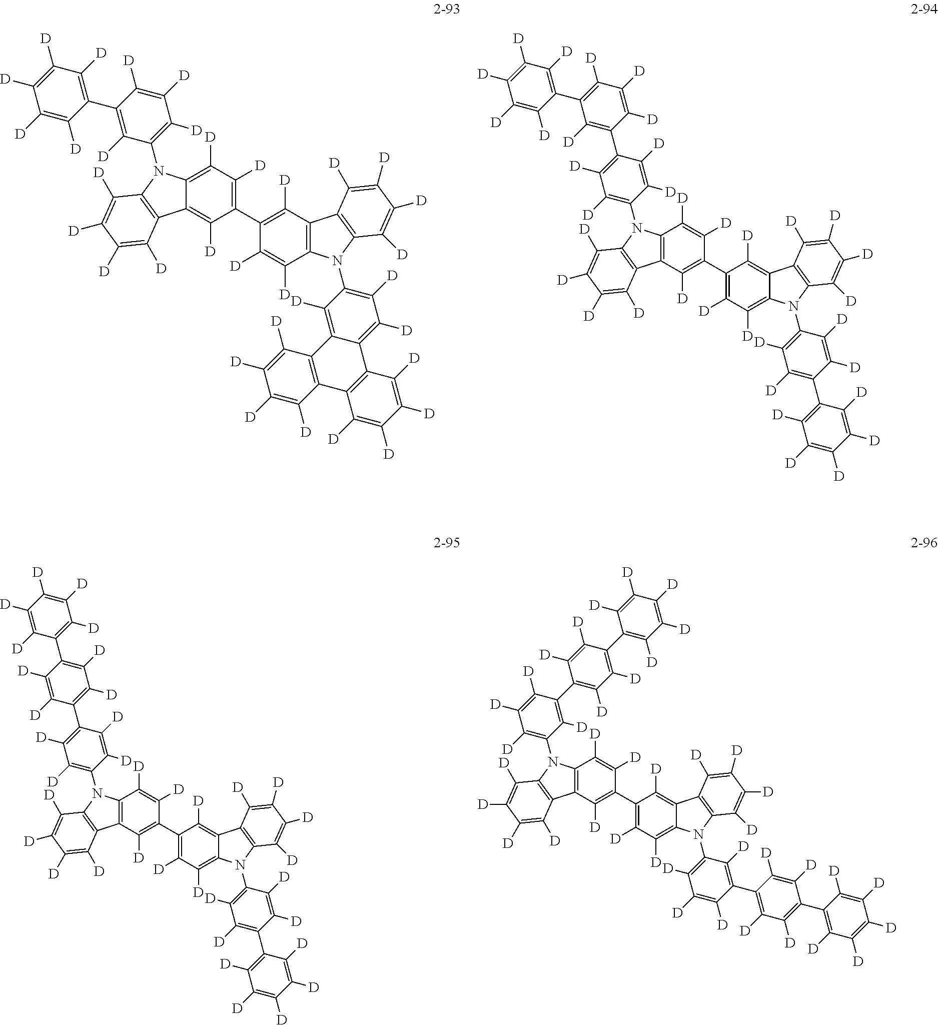

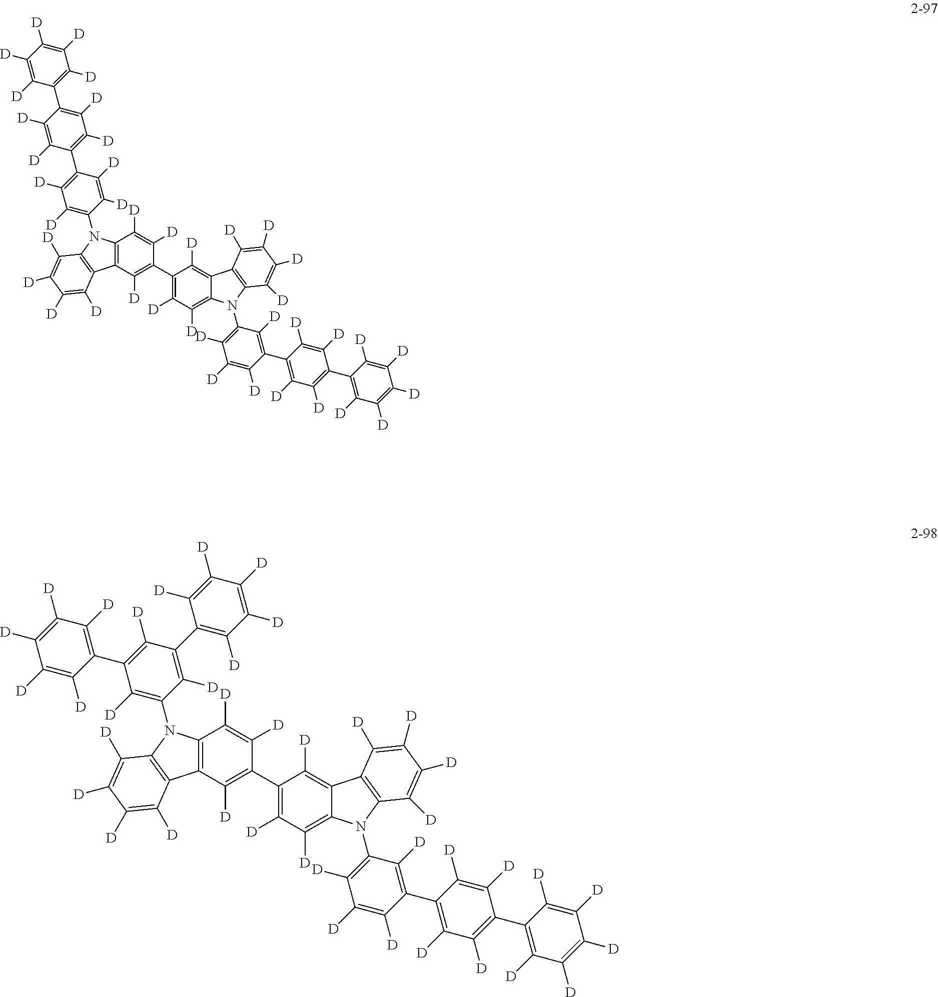

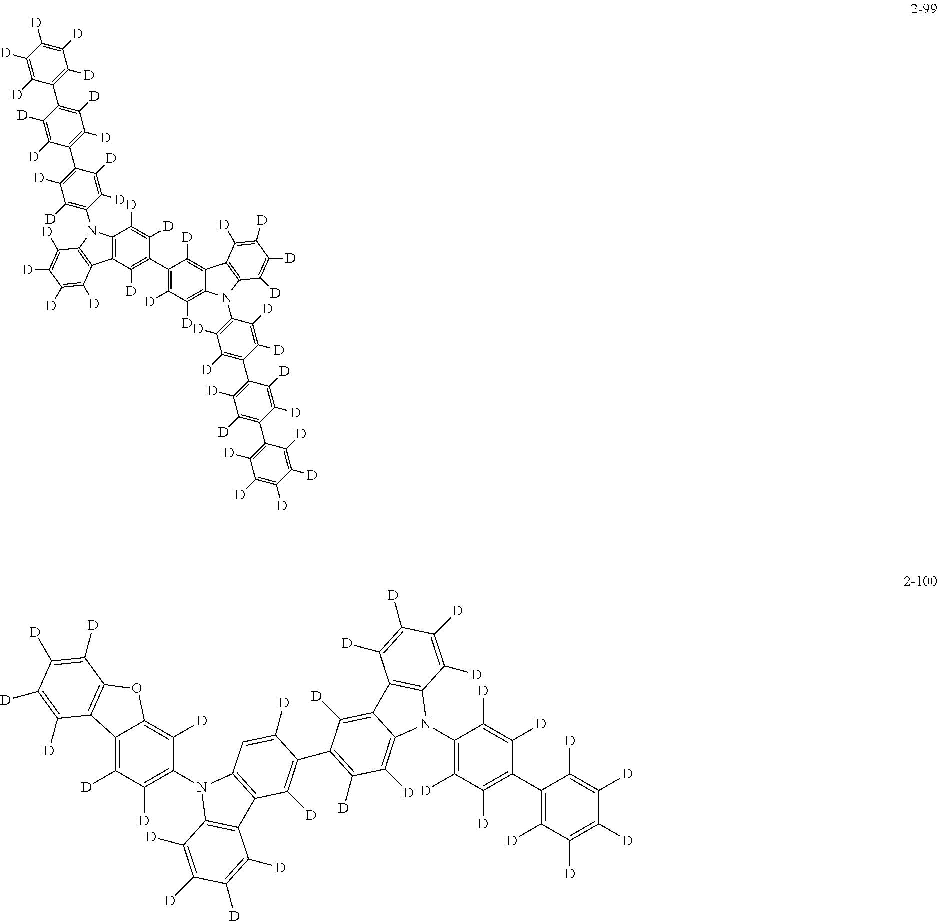

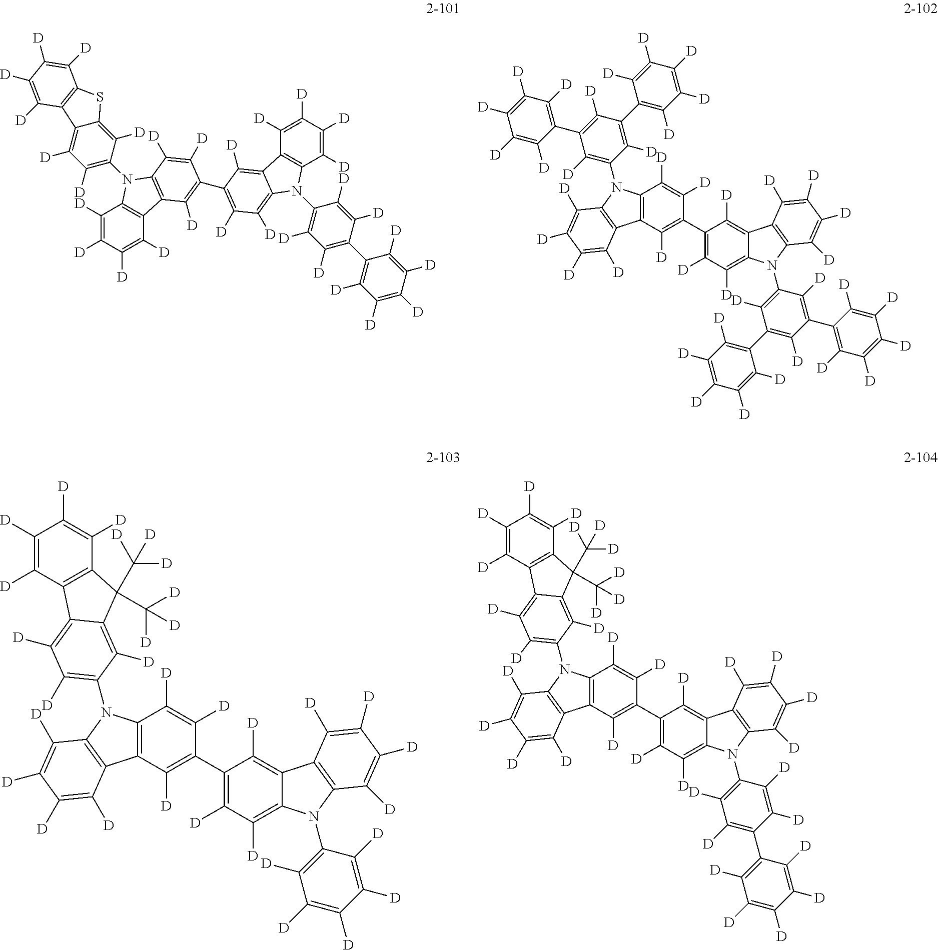

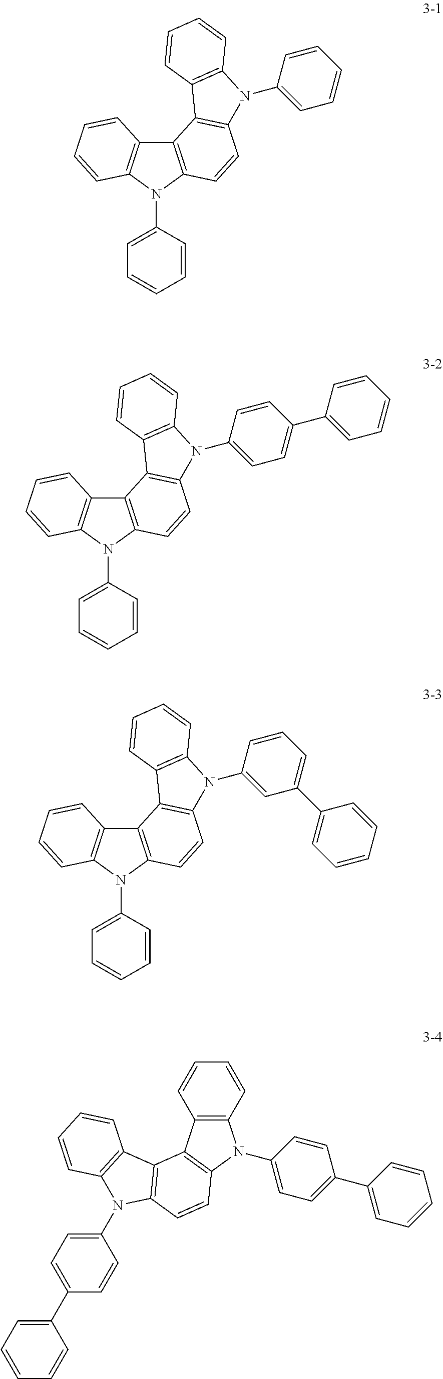 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccc(-c7ccccc7)cc6)c5ccc43)cc2)cc1]

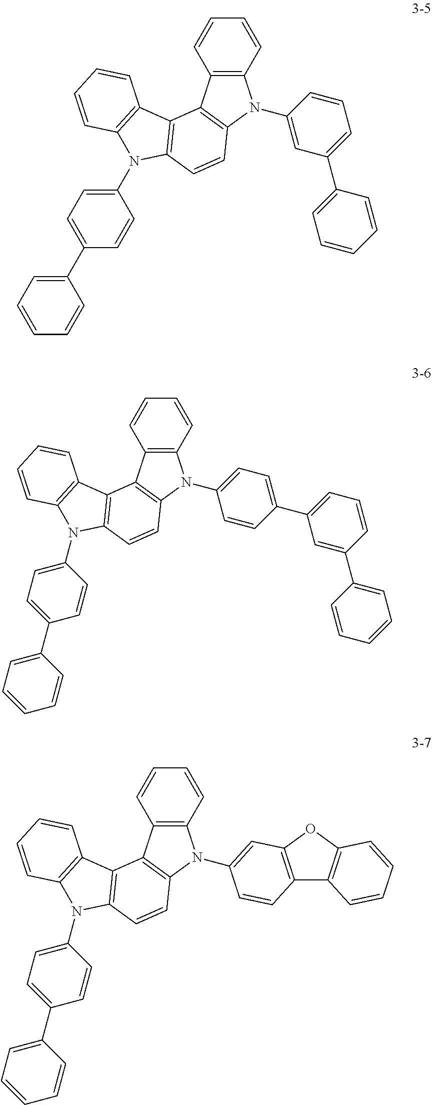 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6ccc(-c7cccc(-c8ccccc8)c7)cc6)c5ccc43)cc2)cc1 ; c1ccc(-c2ccc(-n3c4ccccc4c4c5c6ccccc6n(-c6ccc7c(c6)oc6ccccc67)c5ccc43)cc2)cc1]

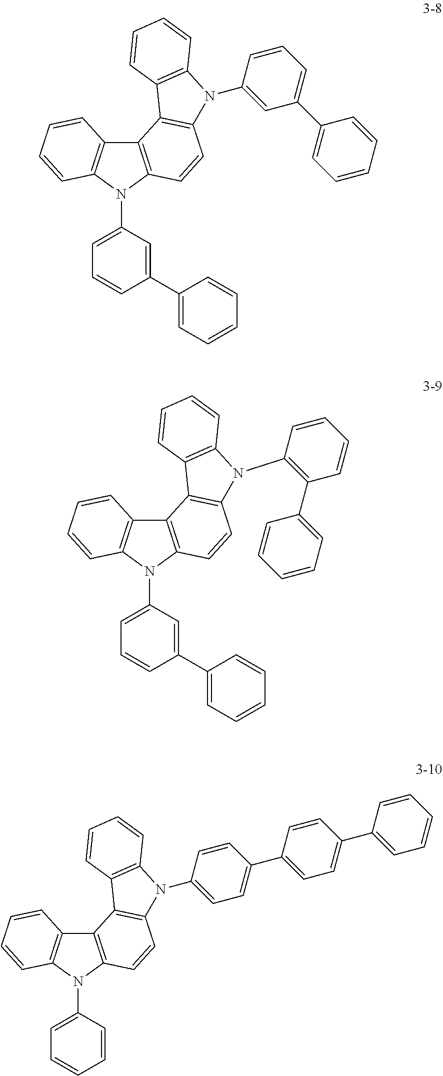 [SMILES: c1ccc(-c2cccc(-n3c4ccccc4c4c5c6ccccc6n(-c6cccc(-c7ccccc7)c6)c5ccc43)c2)cc1 ; c1ccc(-c2cccc(-n3c4ccccc4c4c5c6ccccc6n(-c6ccccc6-c6ccccc6)c5ccc43)c2)cc1 ; c1ccc(-c2ccc(-c3ccc(-n4c5ccccc5c5c6c7ccccc7n(-c7ccccc7)c6ccc54)cc3)cc2)cc1]

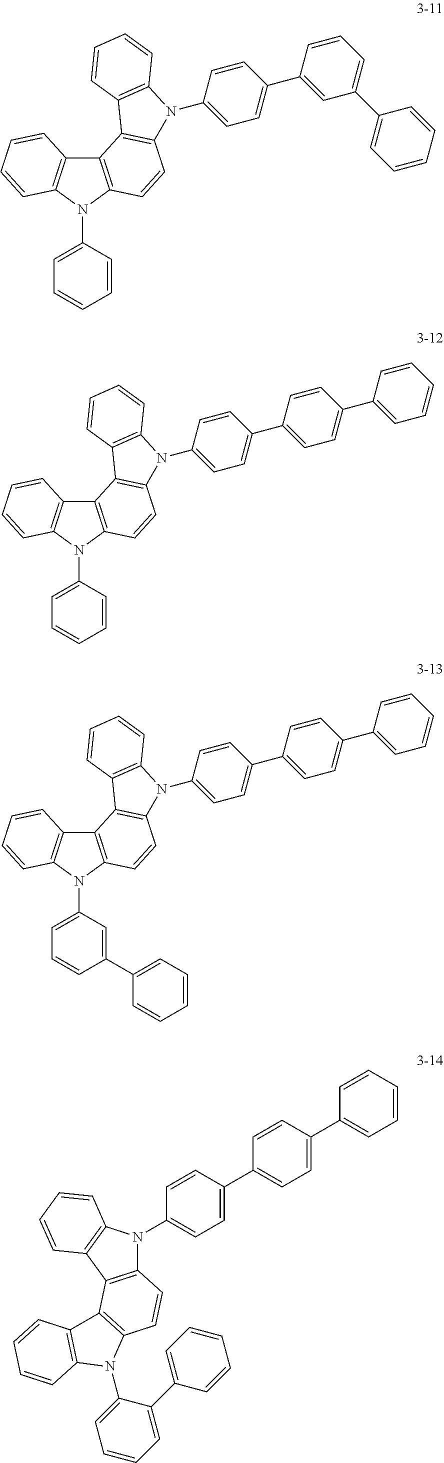 [SMILES: c1ccc(-c2cccc(-c3ccc(-n4c5ccccc5c5c6c7ccccc7n(-c7ccccc7)c6ccc54)cc3)c2)cc1 ; c1ccc(-c2ccc(-c3ccc(-n4c5ccccc5c5c6c7ccccc7n(-c7ccccc7)c6ccc54)cc3)cc2)cc1 ; c1ccc(-c2ccc(-c3ccc(-n4c5ccccc5c5c6c7ccccc7n(-c7cccc(-c8ccccc8)c7)c6ccc54)cc3)cc2)cc1 ; c1ccc(-c2ccc(-c3ccc(-n4c5ccccc5c5c6c7ccccc7n(-c7ccccc7-c7ccccc7)c6ccc54)cc3)cc2)cc1]

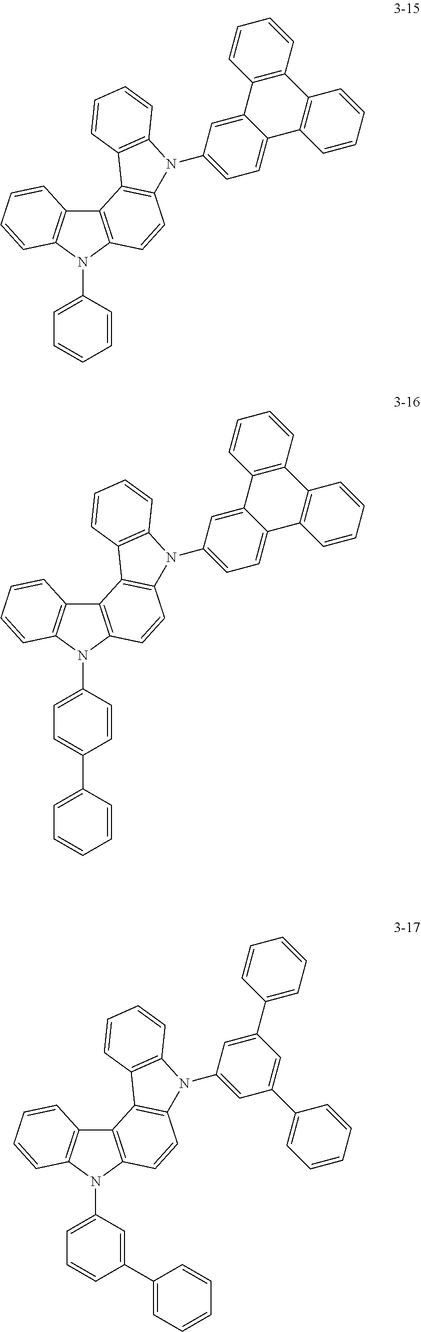 [SMILES: c1ccc(-n2c3ccccc3c3c4c5ccccc5n(-c5ccc6c7ccccc7c7ccccc7c6c5)c4ccc32)cc1 ; c1ccc(-c2ccc(-n3c4ccccc4c4c5c6ccccc6n(-c6ccc7c8ccccc8c8ccccc8c7c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1]

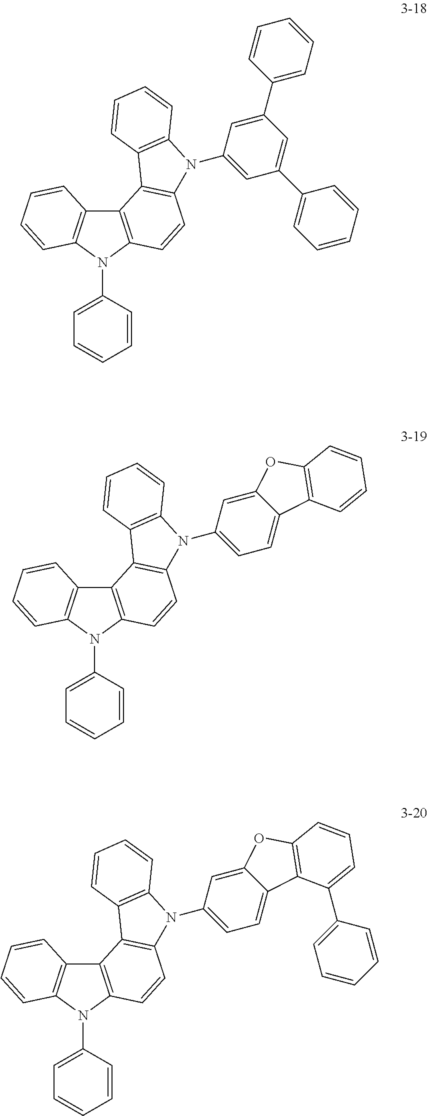 [SMILES: c1ccc(-c2cc(-c3ccccc3)cc(-n3c4ccccc4c4c5c6ccccc6n(-c6ccccc6)c5ccc43)c2)cc1 ; c1ccc(-n2c3ccccc3c3c4c5ccccc5n(-c5ccc6c(c5)oc5ccccc56)c4ccc32)cc1 ; c1ccc(-c2cccc3oc4cc(-n5c6ccccc6c6c7c8ccccc8n(-c8ccccc8)c7ccc65)ccc4c23)cc1]

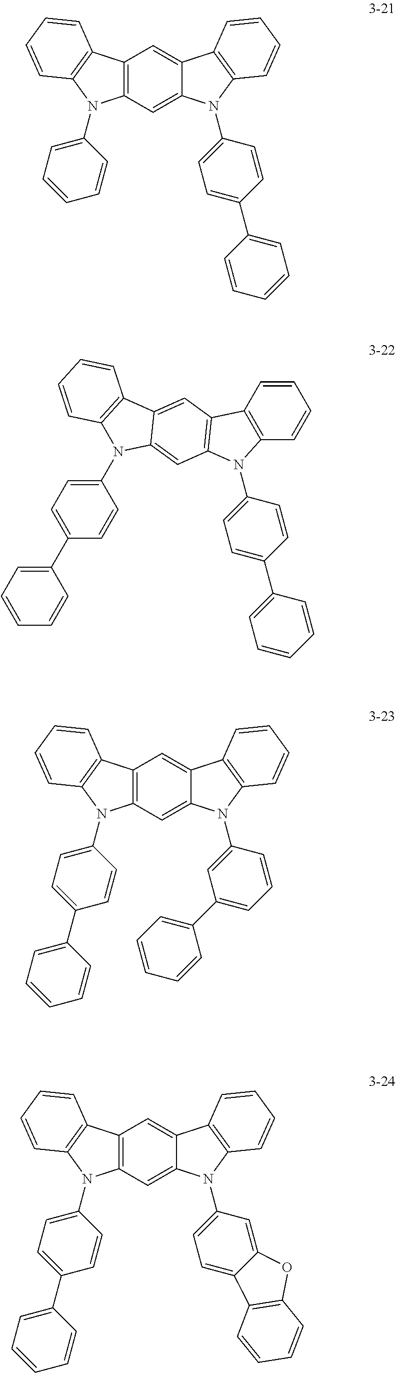 [SMILES: c1ccc(-c2ccc(-n3c4ccccc4c4cc5c6ccccc6n(-c6ccccc6)c5cc43)cc2)cc1 ; c1ccc(-c2ccc(-n3c4ccccc4c4cc5c6ccccc6n(-c6ccc(-c7ccccc7)cc6)c5cc43)cc2)cc1 ; c1ccc(-c2ccc(-n3c4ccccc4c4cc5c6ccccc6n(-c6cccc(-c7ccccc7)c6)c5cc43)cc2)cc1 ; c1ccc(-c2ccc(-n3c4ccccc4c4cc5c6ccccc6n(-c6ccc7c(c6)oc6ccccc67)c5cc43)cc2)cc1]

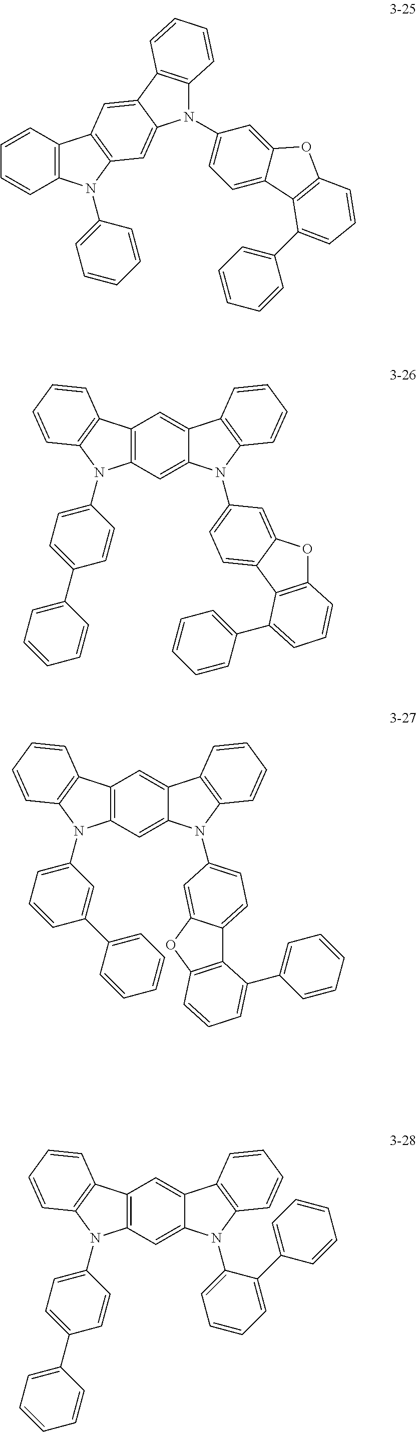 [SMILES: c1ccc(-c2cccc3oc4cc(-n5c6ccccc6c6cc7c8ccccc8n(-c8ccccc8)c7cc65)ccc4c23)cc1 ; c1ccc(-c2ccc(-n3c4ccccc4c4cc5c6ccccc6n(-c6ccc7c(c6)oc6cccc(-c8ccccc8)c67)c5cc43)cc2)cc1 ; c1ccc(-c2cccc(-n3c4ccccc4c4cc5c6ccccc6n(-c6ccc7c(c6)oc6cccc(-c8ccccc8)c67)c5cc43)c2)cc1 ; c1ccc(-c2ccc(-n3c4ccccc4c4cc5c6ccccc6n(-c6ccccc6-c6ccccc6)c5cc43)cc2)cc1]

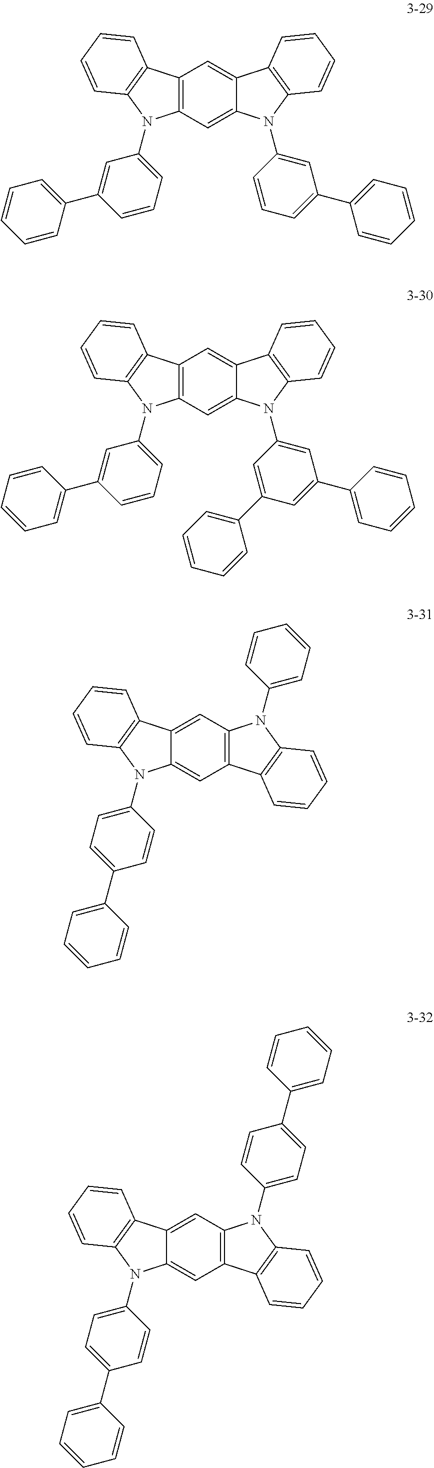 [SMILES: c1ccc(-c2cccc(-n3c4ccccc4c4cc5c6ccccc6n(-c6cccc(-c7ccccc7)c6)c5cc43)c2)cc1 ; c1ccc(-c2cccc(-n3c4ccccc4c4cc5c6ccccc6n(-c6cc(-c7ccccc7)cc(-c7ccccc7)c6)c5cc43)c2)cc1 ; c1ccc(-c2ccc(-n3c4ccccc4c4cc5c(cc43)c3ccccc3n5-c3ccccc3)cc2)cc1 ; c1ccc(-c2ccc(-n3c4ccccc4c4cc5c(cc43)c3ccccc3n5-c3ccc(-c4ccccc4)cc3)cc2)cc1]

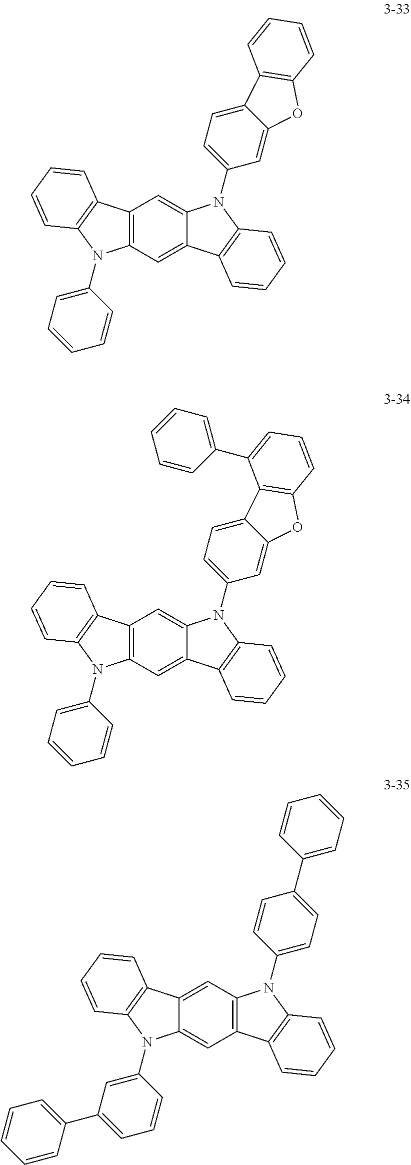 [SMILES: c1ccc(-n2c3ccccc3c3cc4c(cc32)c2ccccc2n4-c2ccc3c(c2)oc2ccccc23)cc1 ; c1ccc(-c2cccc3oc4cc(-n5c6ccccc6c6cc7c(cc65)c5ccccc5n7-c5ccccc5)ccc4c23)cc1 ; c1ccc(-c2ccc(-n3c4ccccc4c4cc5c(cc43)c3ccccc3n5-c3cccc(-c4ccccc4)c3)cc2)cc1]

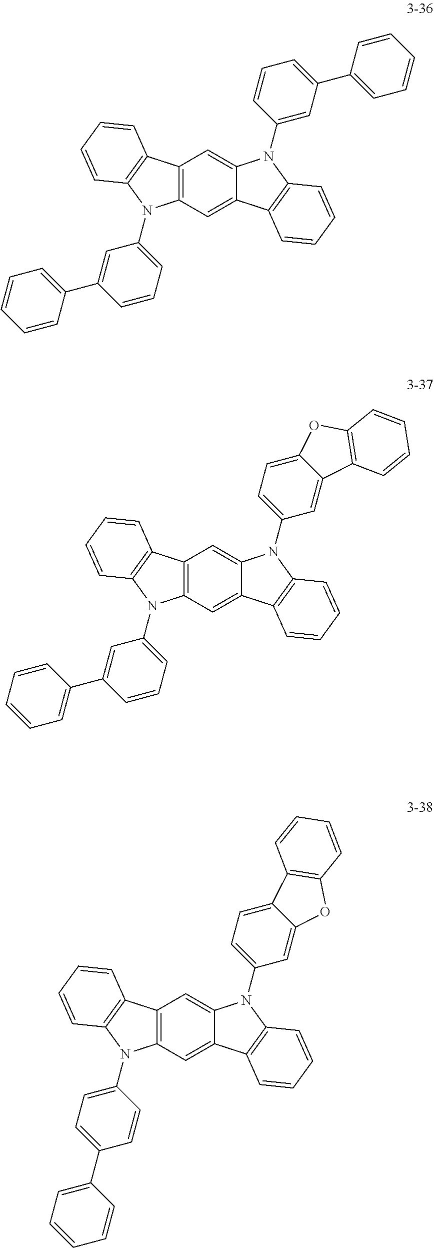 [SMILES: c1ccc(-c2cccc(-n3c4ccccc4c4cc5c(cc43)c3ccccc3n5-c3cccc(-c4ccccc4)c3)c2)cc1 ; c1ccc(-c2cccc(-n3c4ccccc4c4cc5c(cc43)c3ccccc3n5-c3ccc4oc5ccccc5c4c3)c2)cc1 ; c1ccc(-c2ccc(-n3c4ccccc4c4cc5c(cc43)c3ccccc3n5-c3ccc4c(c3)oc3ccccc34)cc2)cc1]

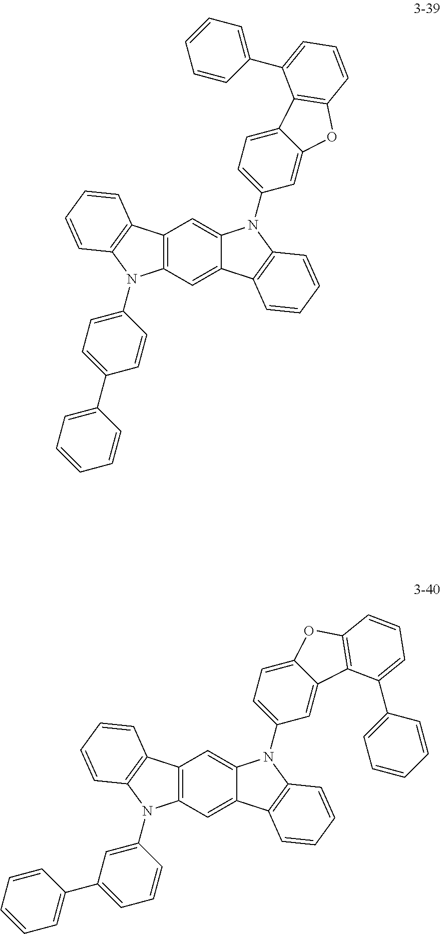 [SMILES: c1ccc(-c2ccc(-n3c4ccccc4c4cc5c(cc43)c3ccccc3n5-c3ccc4c(c3)oc3cccc(-c5ccccc5)c34)cc2)cc1 ; c1ccc(-c2cccc(-n3c4ccccc4c4cc5c(cc43)c3ccccc3n5-c3ccc4oc5cccc(-c6ccccc6)c5c4c3)c2)cc1]

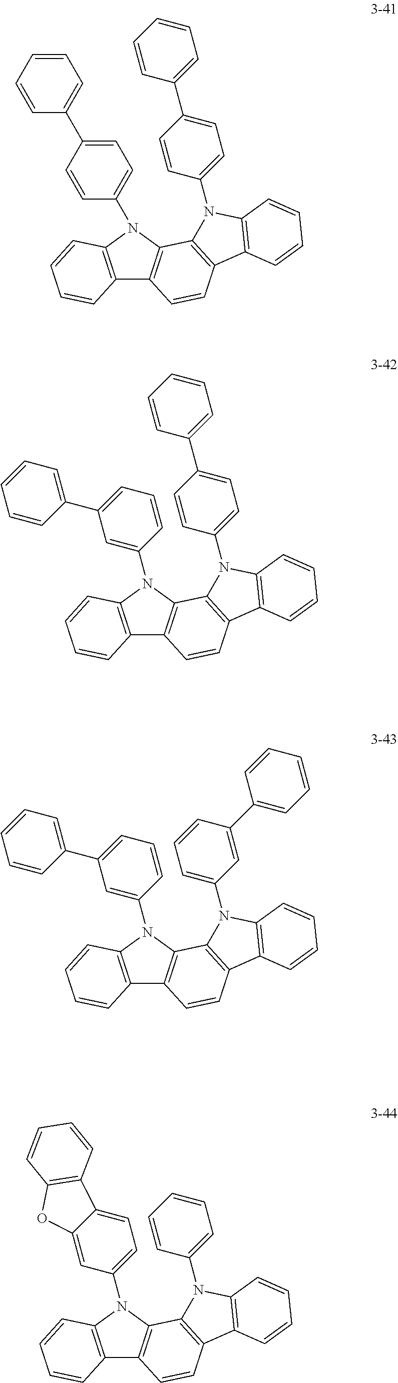 [SMILES: c1ccc(-c2ccc(-n3c4ccccc4c4ccc5c6ccccc6n(-c6ccc(-c7ccccc7)cc6)c5c43)cc2)cc1 ; c1ccc(-c2ccc(-n3c4ccccc4c4ccc5c6ccccc6n(-c6cccc(-c7ccccc7)c6)c5c43)cc2)cc1 ; c1ccc(-c2cccc(-n3c4ccccc4c4ccc5c6ccccc6n(-c6cccc(-c7ccccc7)c6)c5c43)c2)cc1 ; c1ccc(-n2c3ccccc3c3ccc4c5ccccc5n(-c5ccc6c(c5)oc5ccccc56)c4c32)cc1]

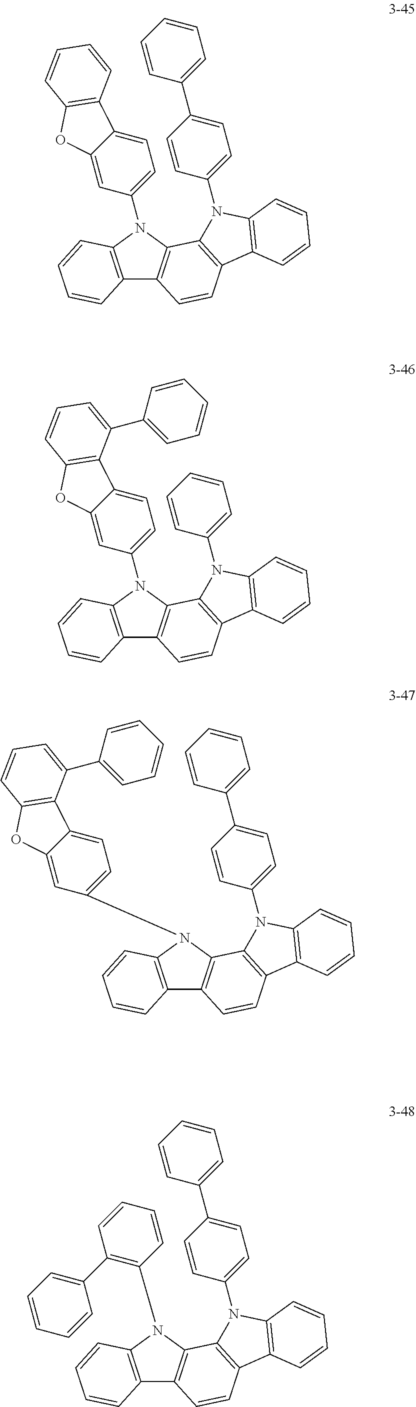 [SMILES: c1ccc(-c2ccc(-n3c4ccccc4c4ccc5c6ccccc6n(-c6ccc7c(c6)oc6ccccc67)c5c43)cc2)cc1 ; c1ccc(-c2cccc3oc4cc(-n5c6ccccc6c6ccc7c8ccccc8n(-c8ccccc8)c7c65)ccc4c23)cc1 ; c1ccc(-c2ccc(-n3c4ccccc4c4ccc5c6ccccc6n(-c6ccc7c(c6)oc6cccc(-c8ccccc8)c67)c5c43)cc2)cc1 ; c1ccc(-c2ccc(-n3c4ccccc4c4ccc5c6ccccc6n(-c6ccccc6-c6ccccc6)c5c43)cc2)cc1]

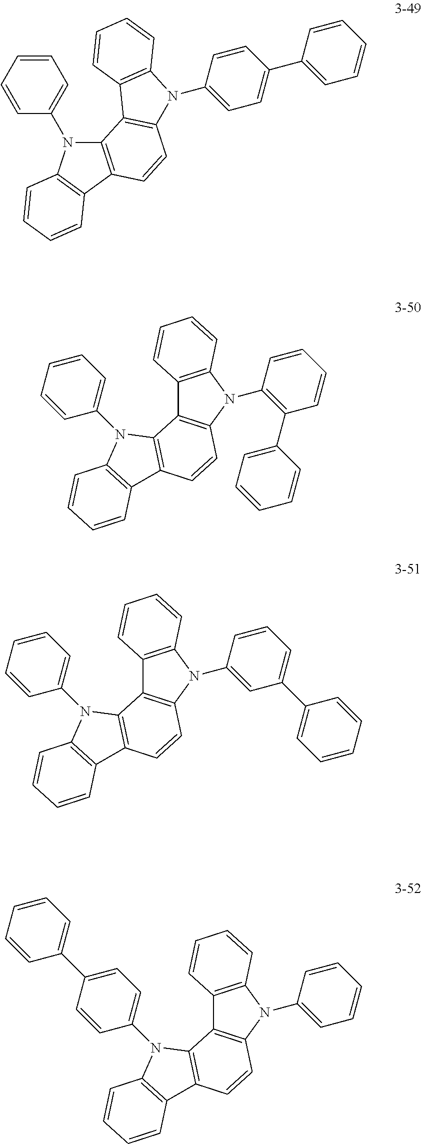 [SMILES: c1ccc(-c2ccc(-n3c4ccccc4c4c3ccc3c5ccccc5n(-c5ccccc5)c34)cc2)cc1 ; c1ccc(-c2ccccc2-n2c3ccccc3c3c2ccc2c4ccccc4n(-c4ccccc4)c23)cc1 ; c1ccc(-c2cccc(-n3c4ccccc4c4c3ccc3c5ccccc5n(-c5ccccc5)c34)c2)cc1 ; c1ccc(-c2ccc(-n3c4ccccc4c4ccc5c(c6ccccc6n5-c5ccccc5)c43)cc2)cc1]

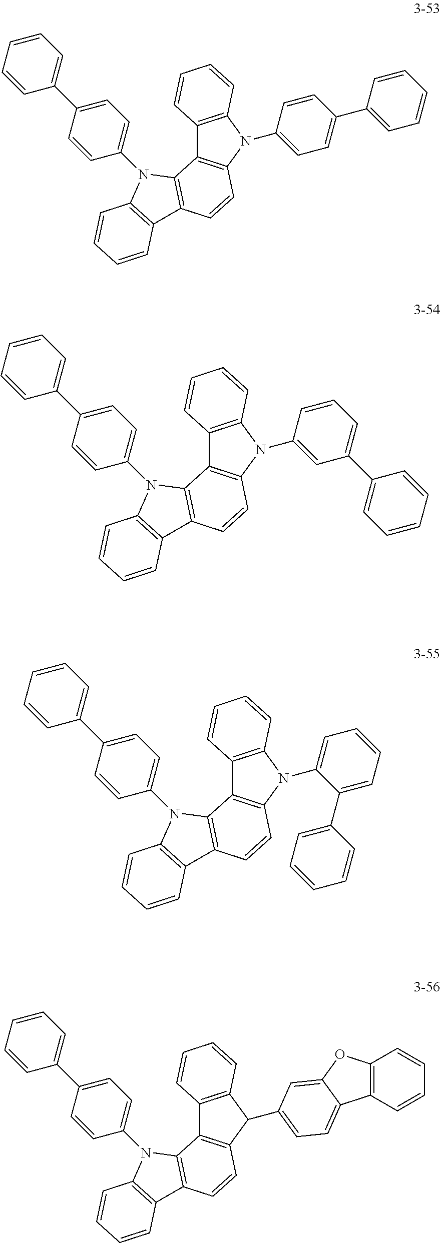 [SMILES: c1ccc(-c2ccc(-n3c4ccccc4c4c3ccc3c5ccccc5n(-c5ccc(-c6ccccc6)cc5)c34)cc2)cc1 ; c1ccc(-c2ccc(-n3c4ccccc4c4ccc5c(c6ccccc6n5-c5cccc(-c6ccccc6)c5)c43)cc2)cc1 ; c1ccc(-c2ccc(-n3c4ccccc4c4ccc5c(c6ccccc6n5-c5ccccc5-c5ccccc5)c43)cc2)cc1 ; c1ccc(-c2ccc(-n3c4ccccc4c4ccc5c(c43)-c3ccccc3C5c3ccc4c(c3)oc3ccccc34)cc2)cc1]

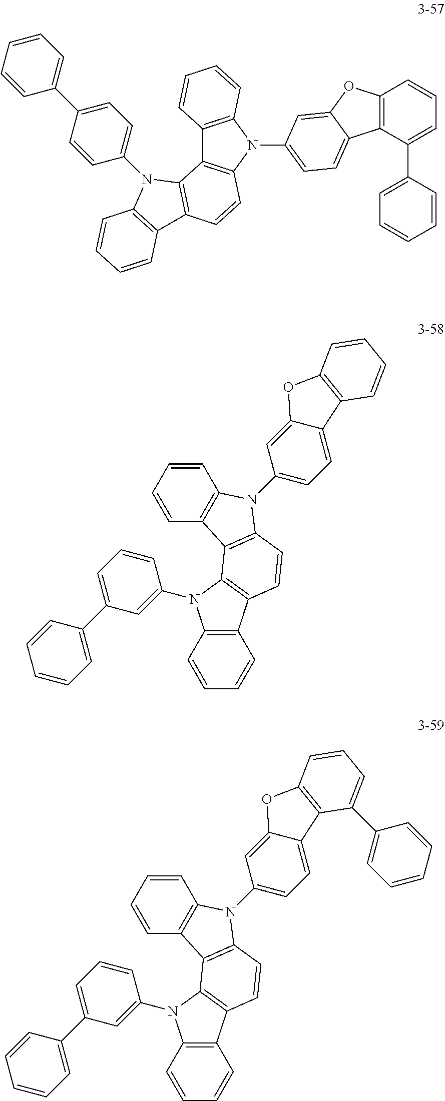 [SMILES: c1ccc(-c2ccc(-n3c4ccccc4c4ccc5c(c6ccccc6n5-c5ccc6c(c5)oc5cccc(-c7ccccc7)c56)c43)cc2)cc1 ; c1ccc(-c2cccc(-n3c4ccccc4c4ccc5c(c6ccccc6n5-c5ccc6c(c5)oc5ccccc56)c43)c2)cc1 ; c1ccc(-c2cccc(-n3c4ccccc4c4ccc5c(c6ccccc6n5-c5ccc6c(c5)oc5cccc(-c7ccccc7)c56)c43)c2)cc1]

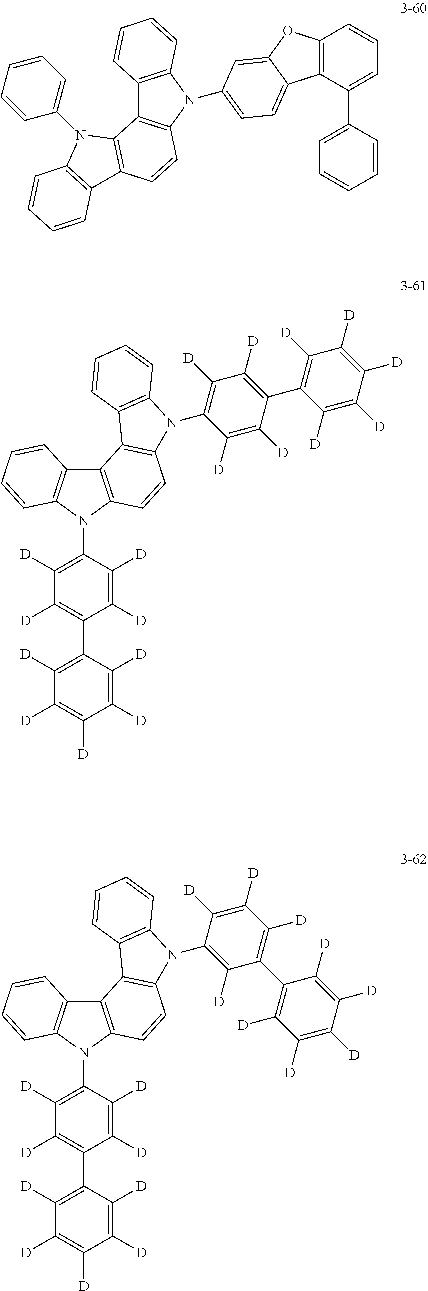 [SMILES: c1ccc(-c2cccc3oc4cc(-n5c6ccccc6c6c5ccc5c7ccccc7n(-c7ccccc7)c56)ccc4c23)cc1 ; [2H]c1c([2H])c([2H])c(-c2c([2H])c([2H])c(-n3c4ccccc4c4c5c6ccccc6n(-c6c([2H])c([2H])c(-c7c([2H])c([2H])c([2H])c([2H])c7[2H])c([2H])c6[2H])c5ccc43)c([2H])c2[2H])c([2H])c1[2H] ; [2H]c1c([2H])c([2H])c(-c2c([2H])c([2H])c(-n3c4ccccc4c4c5c6ccccc6n(-c6c([2H])c([2H])c([2H])c(-c7c([2H])c([2H])c([2H])c([2H])c7[2H])c6[2H])c5ccc43)c([2H])c2[2H])c([2H])c1[2H]]

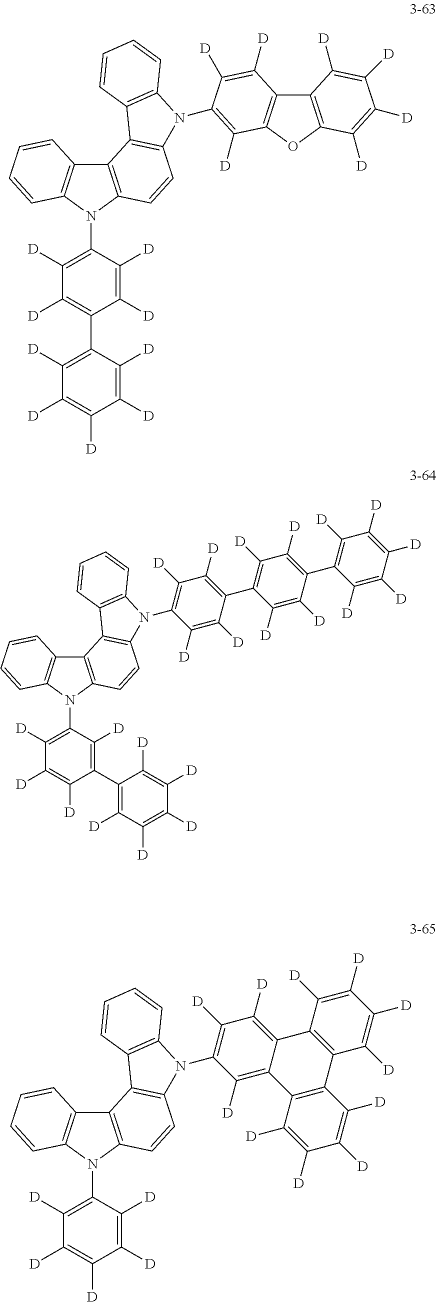

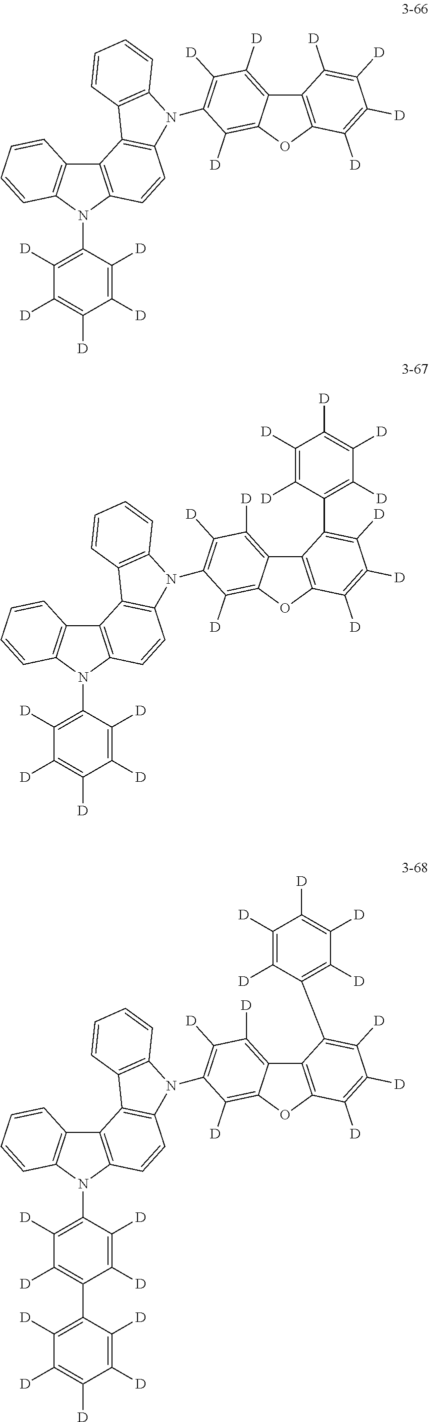 [SMILES: [2H]c1c([2H])c([2H])c(-n2c3ccccc3c3c4c5ccccc5n(-c5c([2H])c([2H])c6c(oc7c([2H])c([2H])c([2H])c([2H])c76)c5[2H])c4ccc32)c([2H])c1[2H] ; [2H]c1c([2H])c([2H])c(-c2c([2H])c([2H])c([2H])c3oc4c([2H])c(-n5c6ccccc6c6c7c8ccccc8n(-c8c([2H])c([2H])c([2H])c([2H])c8[2H])c7ccc65)c([2H])c([2H])c4c23)c([2H])c1[2H] ; [2H]c1c([2H])c([2H])c(-c2c([2H])c([2H])c(-n3c4ccccc4c4c5c6ccccc6n(-c6c([2H])c([2H])c7c(oc8c([2H])c([2H])c([2H])c(-c9c([2H])c([2H])c([2H])c([2H])c9[2H])c87)c6[2H])c5ccc43)c([2H])c2[2H])c([2H])c1[2H]]

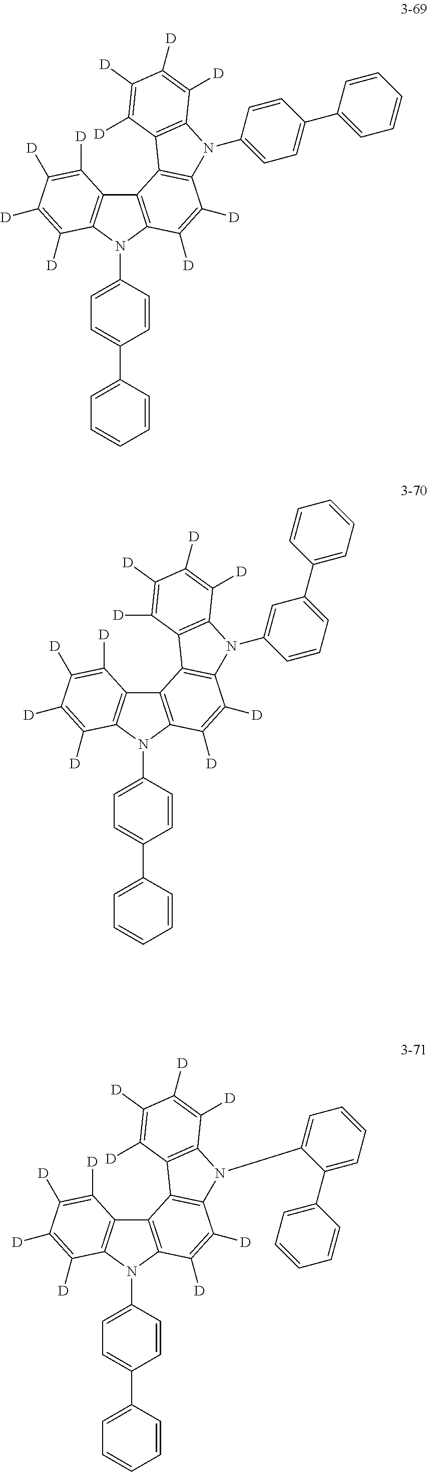 [SMILES: [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1 ; [2H]c1c([2H])c([2H])c2c(c1[2H])c1c3c4c([2H])c([2H])c([2H])c([2H])c4n(-c4cccc(-c5ccccc5)c4)c3c([2H])c([2H])c1n2-c1ccc(-c2ccccc2)cc1 ; [2H]c1c([2H])c([2H])c2c(c1[2H])c1c3c4c([2H])c([2H])c([2H])c([2H])c4n(-c4ccccc4-c4ccccc4)c3c([2H])c([2H])c1n2-c1ccc(-c2ccccc2)cc1]

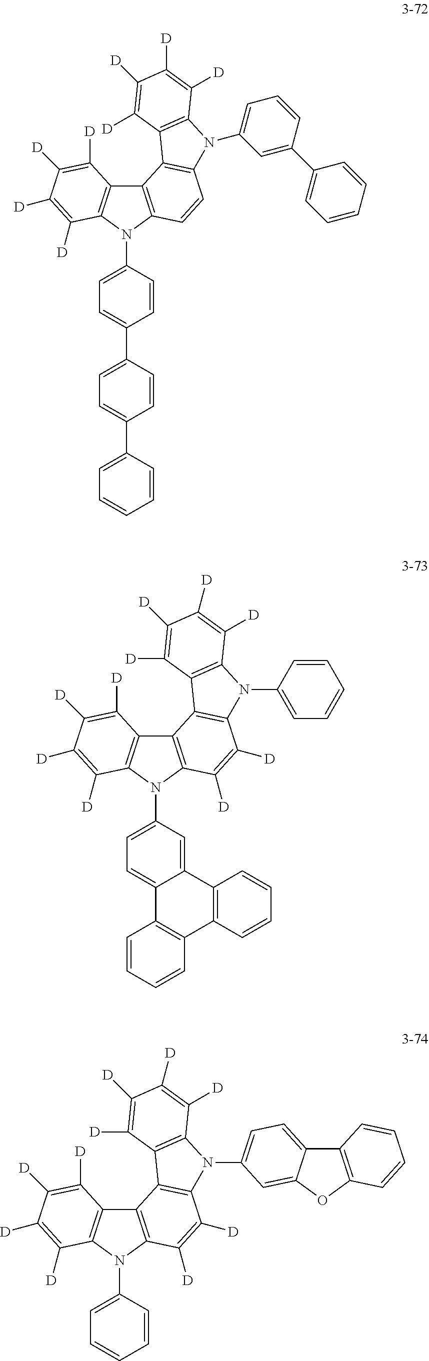 [SMILES: [2H]c1c([2H])c([2H])c2c(c1[2H])c1c3c4c([2H])c([2H])c([2H])c([2H])c4n(-c4cccc(-c5ccccc5)c4)c3ccc1n2-c1ccc(-c2ccc(-c3ccccc3)cc2)cc1 ; [2H]c1c([2H])c([2H])c2c(c1[2H])c1c3c4c([2H])c([2H])c([2H])c([2H])c4n(-c4ccc5c6ccccc6c6ccccc6c5c4)c3c([2H])c([2H])c1n2-c1ccccc1 ; [2H]c1c([2H])c([2H])c2c(c1[2H])c1c3c4c([2H])c([2H])c([2H])c([2H])c4n(-c4ccc5c(c4)oc4ccccc45)c3c([2H])c([2H])c1n2-c1ccccc1]

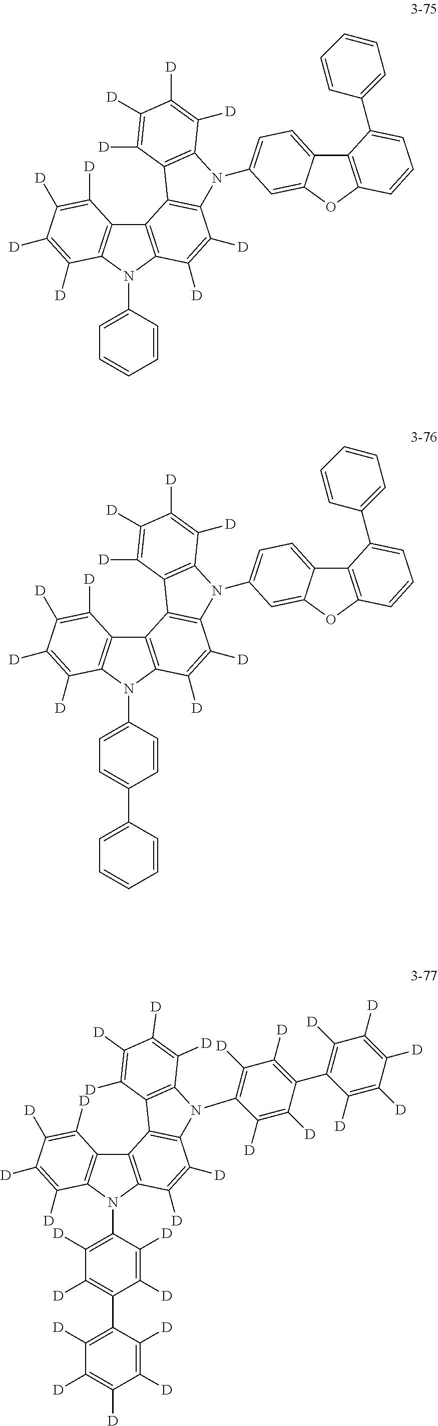

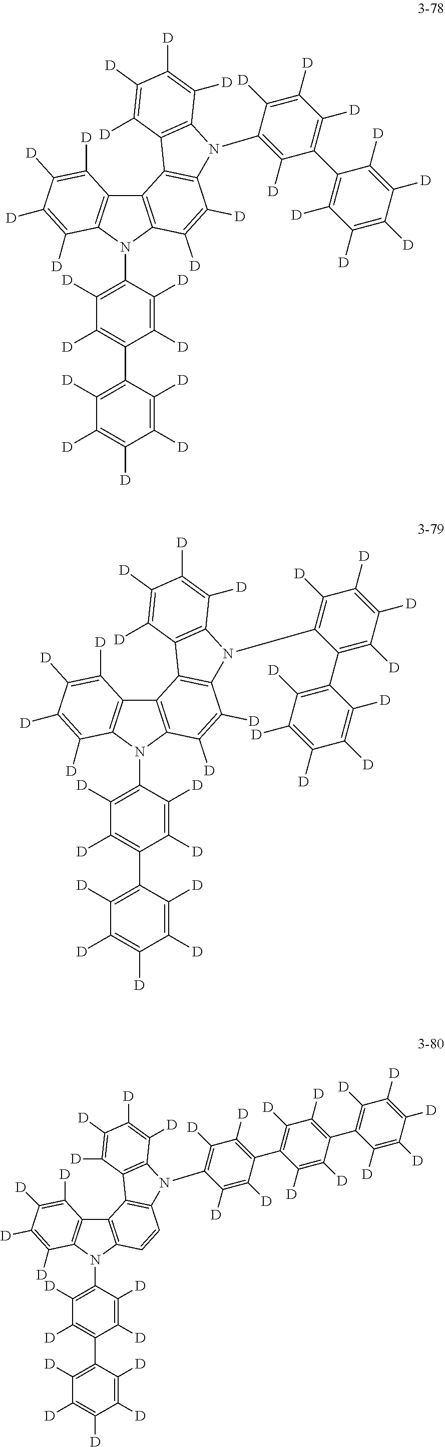

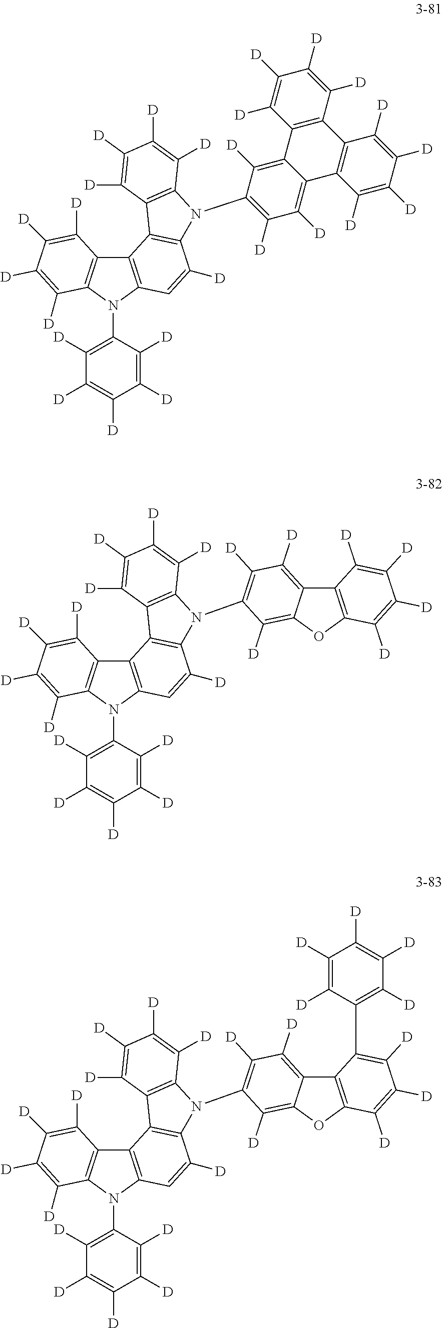

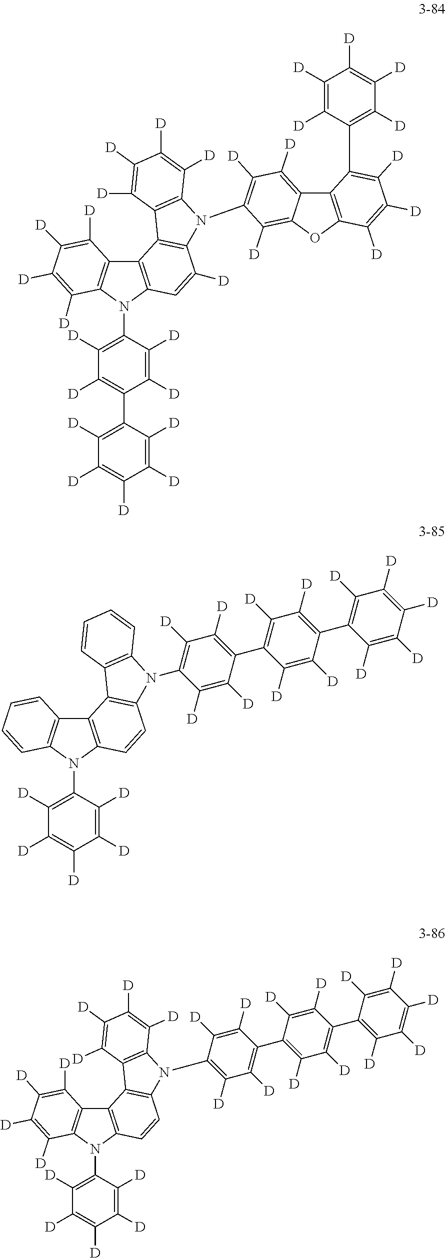

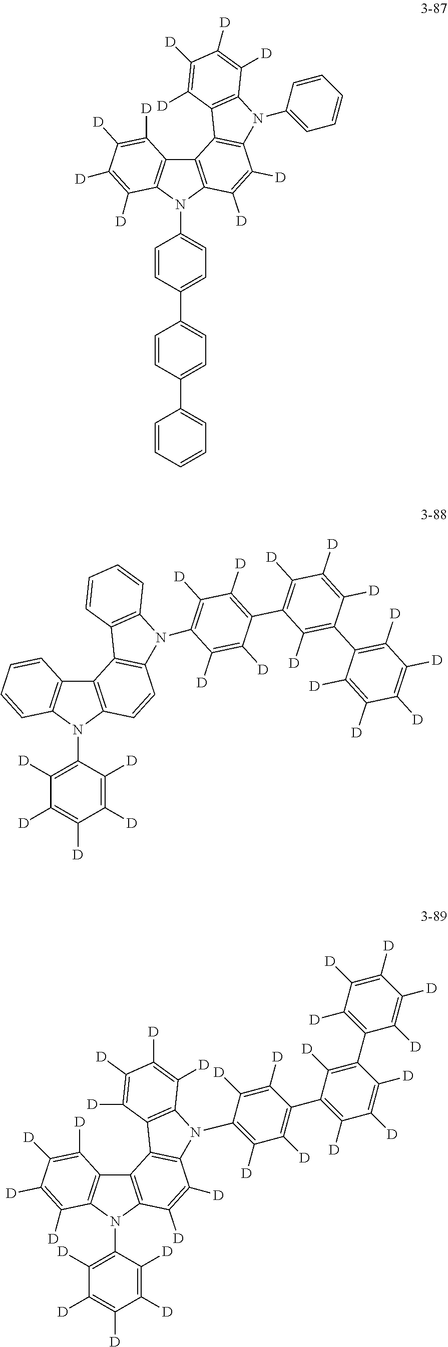

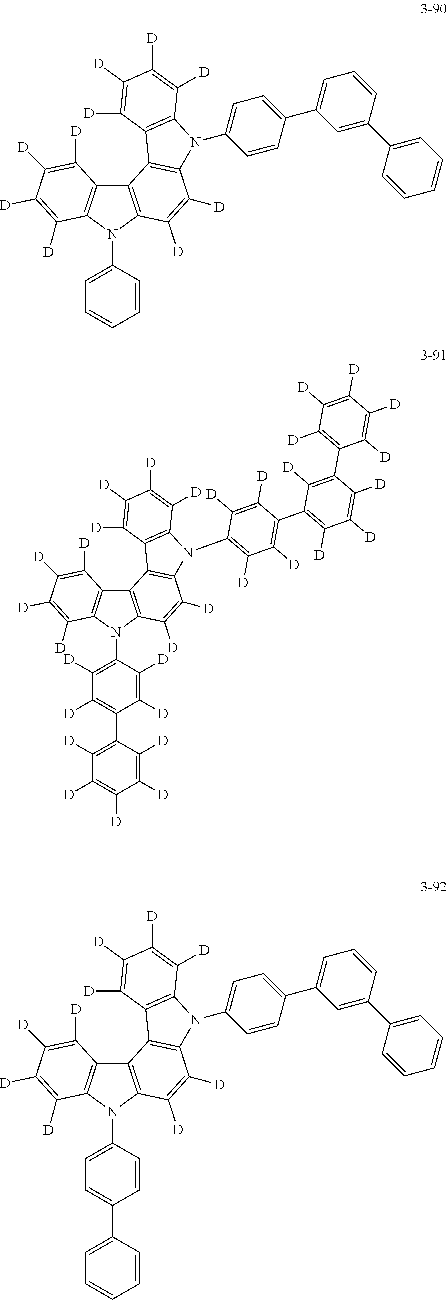

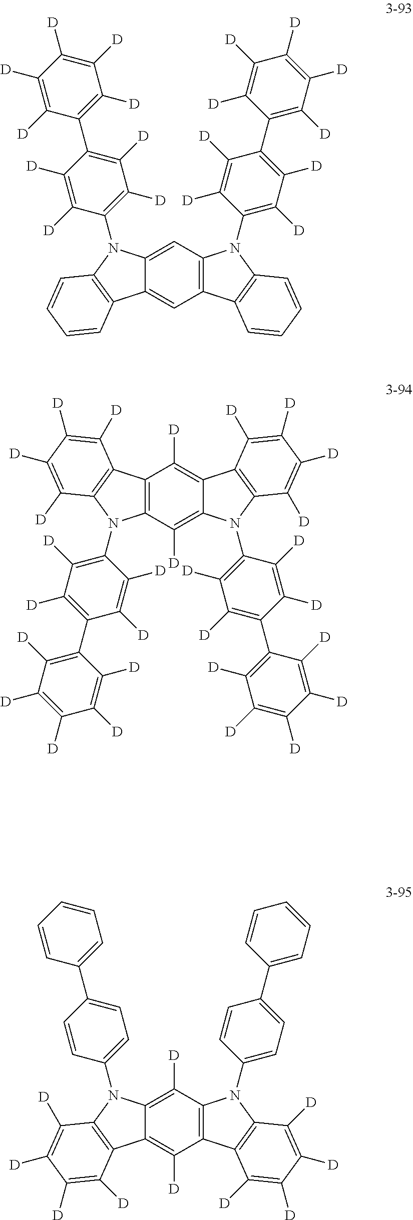

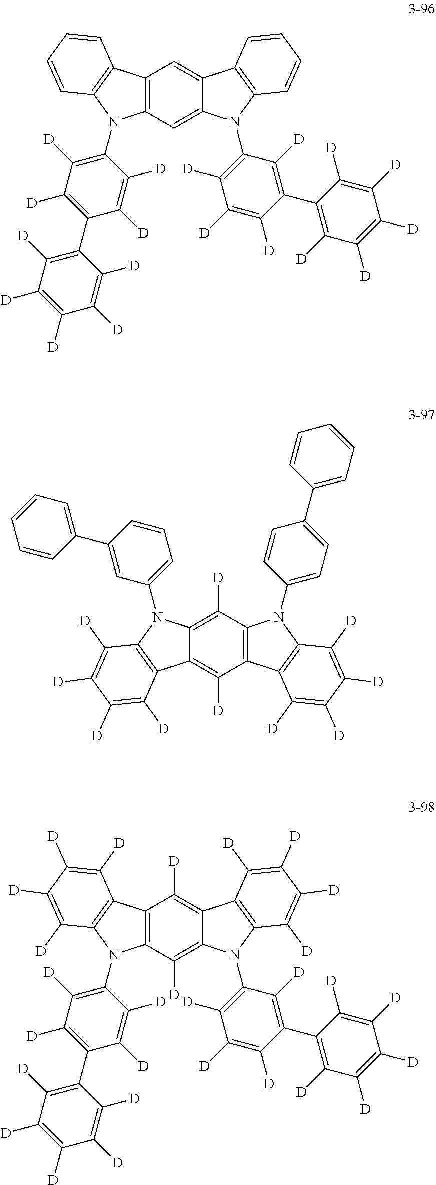

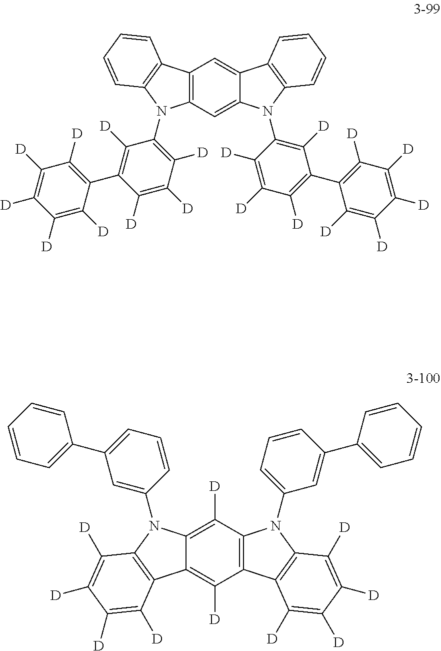 [SMILES: [2H]c1c([2H])c([2H])c(-c2c([2H])c([2H])c([2H])c(-n3c4ccccc4c4cc5c6ccccc6n(-c6c([2H])c([2H])c([2H])c(-c7c([2H])c([2H])c([2H])c([2H])c7[2H])c6[2H])c5cc43)c2[2H])c([2H])c1[2H] ; [2H]c1c([2H])c([2H])c2c(c1[2H])c1c([2H])c3c4c([2H])c([2H])c([2H])c([2H])c4n(-c4cccc(-c5ccccc5)c4)c3c([2H])c1n2-c1cccc(-c2ccccc2)c1]

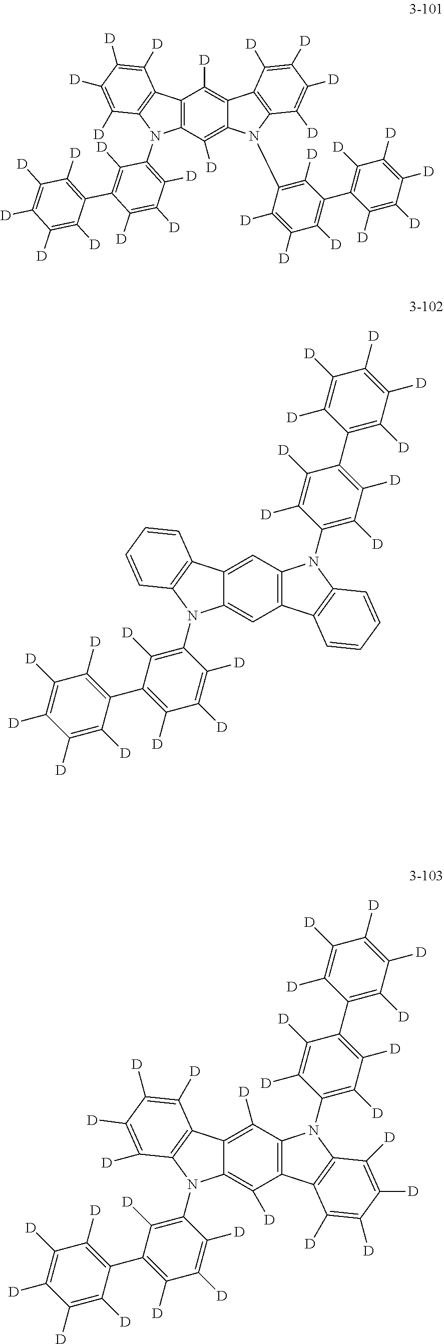

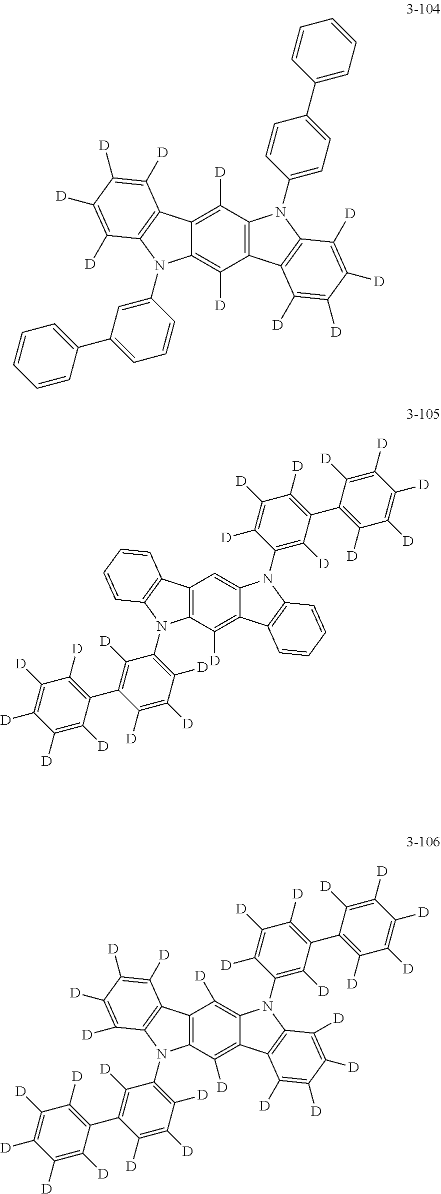

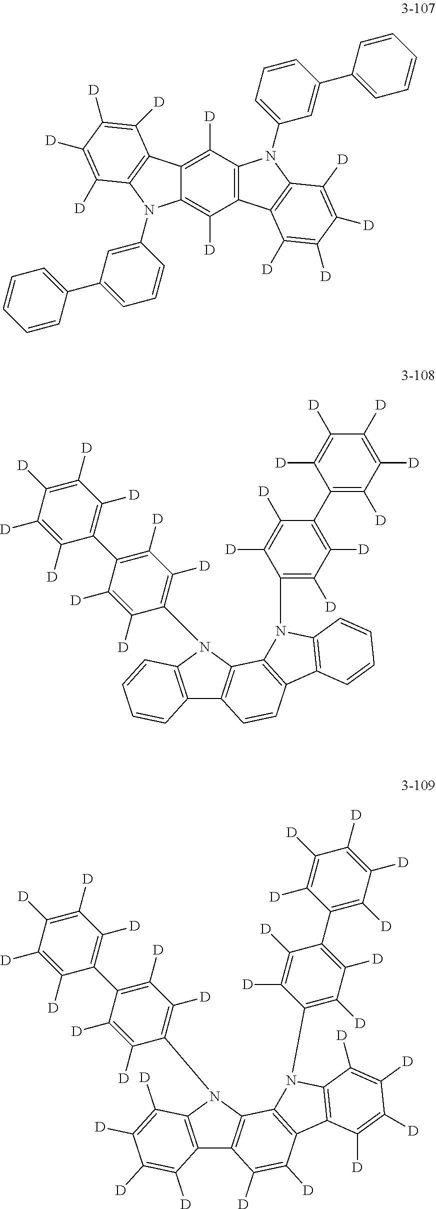

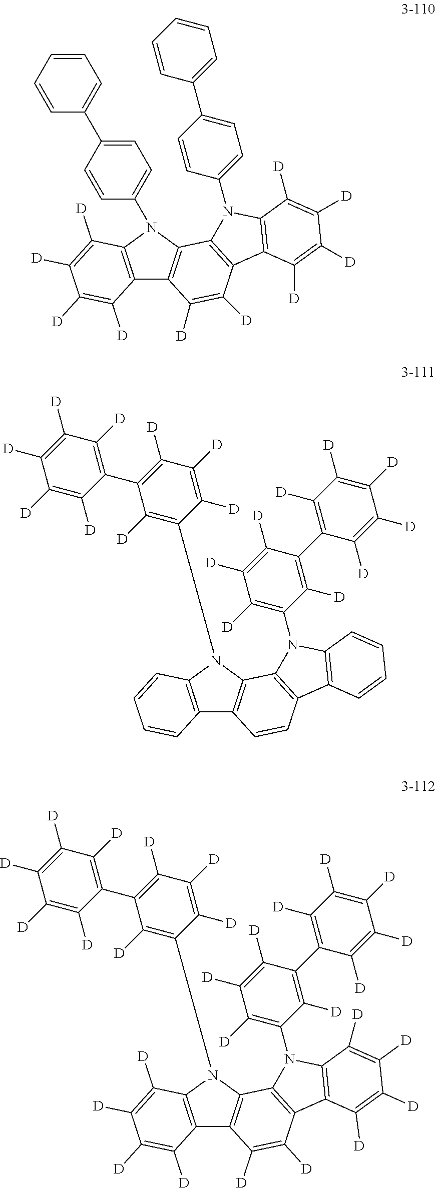

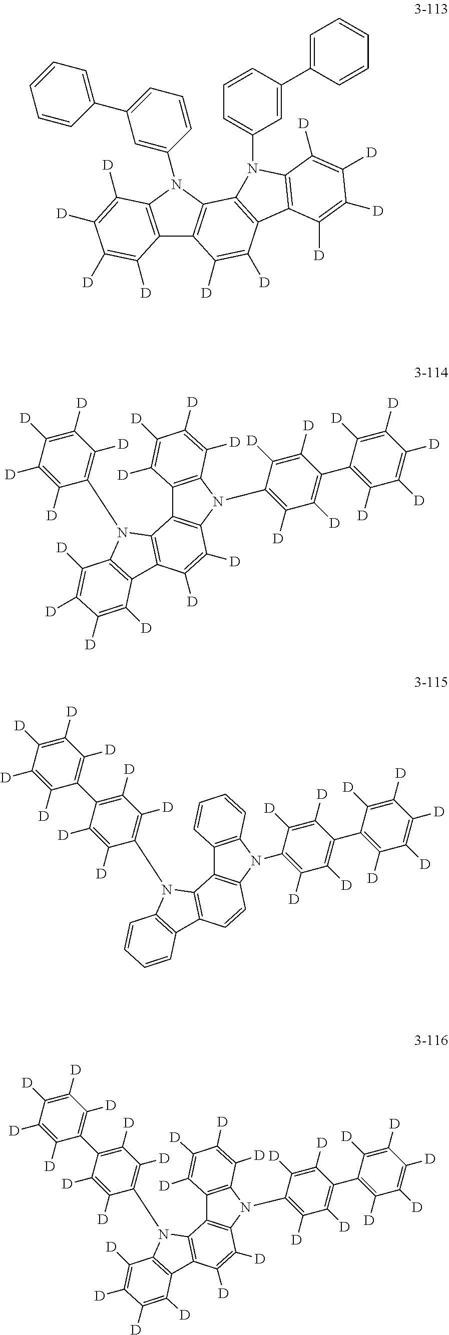

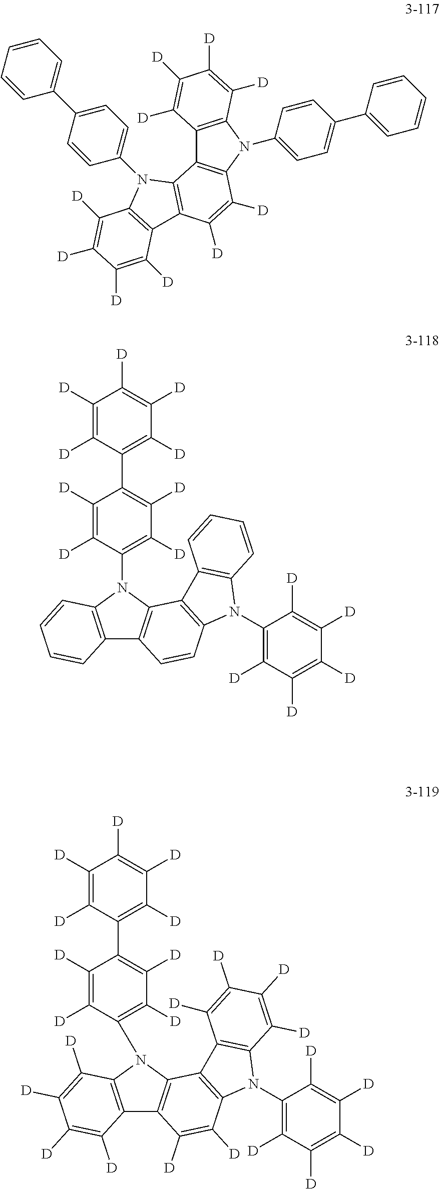 [SMILES: [2H]c1c([2H])c([2H])c2c(c1[2H])c1c(c([2H])c([2H])c3c4c([2H])c([2H])c([2H])c([2H])c4n(-c4ccc(-c5ccccc5)cc4)c31)n2-c1ccc(-c2ccccc2)cc1 ; [2H]c1c([2H])c([2H])c(-c2c([2H])c([2H])c(-n3c4ccccc4c4ccc5c(c6ccccc6n5-c5c([2H])c([2H])c([2H])c([2H])c5[2H])c43)c([2H])c2[2H])c([2H])c1[2H] ; [2H]c1c([2H])c([2H])c(-c2c([2H])c([2H])c(-n3c4c([2H])c([2H])c([2H])c([2H])c4c4c([2H])c([2H])c5c(c6c([2H])c([2H])c([2H])c([2H])c6n5-c5c([2H])c([2H])c([2H])c([2H])c5[2H])c43)c([2H])c2[2H])c([2H])c1[2H]]

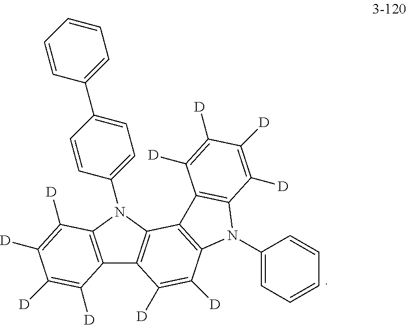 [SMILES: [2H]c1c([2H])c([2H])c2c(c1[2H])c1c(c([2H])c([2H])c3c4c([2H])c([2H])c([2H])c([2H])c4n(-c4ccc(-c5ccccc5)cc4)c31)n2-c1ccccc1]

10. The organic light emitting device of claim 7, wherein Rc and Rd are hydrogen; or deuterium.

11. The organic light emitting device of claim 6, wherein the organic material layer comprises a light emitting layer, and the light emitting layer comprises the heterocyclic compound.

12. The organic light emitting device of claim 6, wherein the organic material layer comprises a light emitting layer, the light emitting layer comprises a host material, and the host material comprises the heterocyclic compound.

13. The organic light emitting device of claim 6, further comprising one or two or more layers selected from the group consisting of a light emitting layer, a hole injection layer, a hole transport layer, an electron injection layer, an electron transport layer, an electron blocking layer and a hole blocking layer.

14. A composition for an organic material layer of an organic light emitting device, comprising the heterocyclic compound according to claim 1 and a heterocyclic compound represented by the following Chemical Formula 2 or 3: 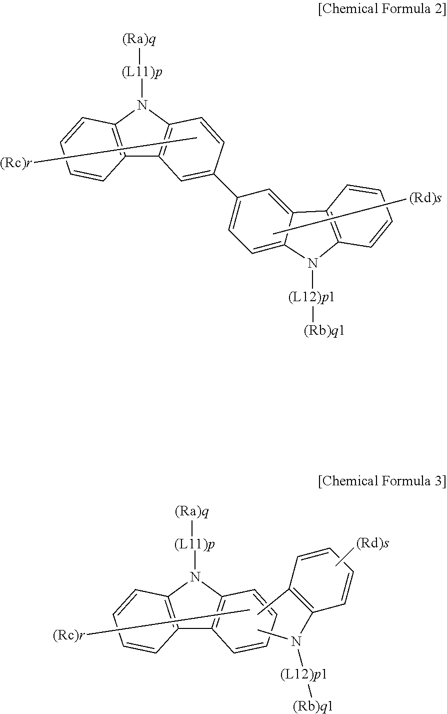
wherein, in Chemical Formulae 2 and 3,
Rc and Rd are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
L11 and L12 are the same as or different from each other, and are each independently a direct bond; a substituted or unsubstituted C6 to C60 arylene group; or a substituted or unsubstituted C2 to C60 heteroarylene group,
Ra and Rb are the same as or different from each other, and are each independently a substituted or unsubstituted C6 to C60 aryl group; a substituted or unsubstituted C2 to C60 heteroaryl group; a cyano group; or —SiRR′R″,
R, R′, and R″ are the same as or different from each other, and are each independently hydrogen; deuterium; —CN; a substituted or unsubstituted C1 to C60 alkyl group; a substituted or unsubstituted C6 to C60 aryl group; or a substituted or unsubstituted C2 to C60 heteroaryl group,
p, p1, q, and q1 are an integer from 1 to 4,
r and s are an integer from 0 to 7,
r1 is an integer from 0 to 4,
s1 is an integer from 0 to 6, and
when p, p1, q, q1, r, s, r1, and s1 are 2 or higher, substituent in the parenthesis are the same as or different from each other.

15. The composition of claim 14, wherein a weight ratio of the heterocyclic compound:the heterocyclic compound represented by Chemical Formula 2 or 3 in the composition is 1:10 to 10:1.
